(19) United States
(12) Patent Application Publication
(10) Pub. No.: US 2009/0118132 A1
(43) Pub. Date: May 7, 2009

(54) Classification of Acute Myeloid Leukemia

(75) Inventors: Torsten Haferlach, Munich (DE); Martin Dugas, Munich (DE); Wolfgang Kern, Starnberg (DE); Alexander Kohlmann, Neumarkt (DE); Susanne Schnittger, Munich (DE); Claudia Schoch, Munich (DE)

Correspondence Address: CLARK & ELBING LLP, 101 FEDERAL STREET, BOSTON, MA, 02110, US

(73) Assignees: Roche Molecular Systems, Inc., Alameda, CA (US); Ludwig-Maximilians-Universitaet, Munichde (DE)

(21) Appl. No.: 11/666,648

(22) PCT Filed: Nov. 3, 2005
(86) PCT No.: PCT/EP05/11728

§ 371 Date: Apr. 30, 2007

Related U.S. Application Data
(60) Provisional application No. 60/625,314, filed on Nov. 4, 2004.
(60) Provisional application No. 60/625,692, filed on Nov. 4, 2004.
(60) Provisional application No. 60/625,623, filed on Nov. 4, 2004.
(60) Provisional application No. 60/625,696, filed on Nov. 4, 2004.
(60) Provisional application No. 60/625,238, filed on Nov. 4, 2004.
(60) Provisional application No. 60/625,266, filed on Nov. 4, 2004.
(60) Provisional application No. 60/625,244, filed on Nov. 4, 2004.

(51) Int. Cl. C40B 30/02; C40B 30/04; C40B 50/02; C40B 60/12; C12Q 1/68

(52) U.S. Cl. 506/8; 506/9; 435/6; 506/24; 506/39

(57) ABSTRACT

The present invention relates to rapid and reliable approaches to leukemia prognostication. In addition to methods, the invention also provides related kits and systems.

### FIELD OF THE INVENTION

The present invention relates to the detection of leukemia and accordingly, provides diagnostic and/or prognostic information in certain embodiments.

### BACKGROUND OF THE INVENTION

Leukemias are generally classified into four different groups or types: acute myeloid (AML), acute lymphatic (ALL), chronic myeloid (CML) and chronic lymphatic leukemia (CLL). Within these groups, several subcategories or subtypes can be identified using various approaches. These different subcategories of leukemia are associated with varying clinical outcomes and therefore can serve as guides to the selection of different treatment strategies. The importance of highly specific classification may be illustrated for AML as a very heterogeneous group of diseases. Effort has been aimed at identifying biological entities and to distinguish and classify subgroups of AML that are associated with, e.g., favorable, intermediate or unfavorable prognoses. In 1976, for example, the FAB classification was proposed by the French-American-British co-operative group that utilizes cytomorphology and cytochemistry to separate AML subgroups according to the morphological appearance of blasts in the blood and bone marrow. In addition, genetic abnormalities occurring in leukemic blasts were recognized as having a major impact on the morphological picture and on prognosis. As a consequence, the karyotype of leukemic blasts is commonly used as an independent prognostic factor regarding response to therapy as well as survival.

A combination of methods is typically used to obtain the diagnostic information in leukemia. To illustrate, the analysis of the morphology and cytochemistry of bone marrow blasts and peripheral blood cells is commonly used to establish a diagnosis. In some cases, for example, immunophenotyping is also utilized to separate an undifferentiated AML from acute lymphoblastic leukemia and from CLL. In certain instances, leukemia subtypes can be diagnosed by cytomorphology alone, but this typically requires that an expert review sample smears. However, genetic analysis based on, e.g., chromosome analysis, fluorescence in situ hybridization (FISH), or reverse transcription PCR (RT-PCR) and immunophenotyping is also generally used to accurately assign cases to the correct category. An aim of these techniques, aside from diagnosis, is to determine the prognosis of the leukemia under consideration. One disadvantage of these methods, however, is that viable cells are generally necessary, as the cells used for genetic analysis need to divide in vitro in order to obtain metaphases for the analysis. Another exemplary problem is the long lag period (e.g., 72 hours) that typically occurs between the receipt of the materials to be analyzed in the laboratory and the generation of results. Furthermore, great experience in preparing chromosomes and analyzing karyotypes is generally needed to obtain correct results in most cases. Using these techniques in combination, hematological malignancies can be separated into CML, CLL, ALL, and AML. Within the latter three disease entities, several prognostically relevant subtypes have been identified. This further sub-classification commonly relies on genetic abnormalities of leukemic blasts and is associated with different prognoses.

The sub-classification of leukemias is used increasingly as a guide to the selection of appropriate therapies. The development of new, specific drugs and treatment approaches often includes the identification of specific subtypes that may benefit from a distinct therapeutic protocol and thus, improve the outcomes of distinct subsets of leukemia. For example, the therapeutic drug (STI571) inhibits the CML specific chimeric tyrosine kinase BCR-ABL generated from the genetic defect observed in CML, the BCR-ABL-rearrangement due to the translocation between chromosomes 9 and 22 (t(9;22) (q34;q11)). In patients treated with this new drug, the therapy response is dramatically higher as compared to other drugs that have previously been used. Another example is a subtype of acute myeloid leukemia, AML M3 and its variant M3v, which both include the karyotype t(15;17)(q22;q11-12). The introduction of all-trans retinoic acid (ATRA) has improved the outcome in this subgroup of patient from about 50% to 85% long-term survivors. Accordingly, the rapid and accurate identification of distinct leukemia subtypes is of consequence to further drug development in addition to diagnostics and prognostics.

According to Golub et al. (Science, 1999, 286, 531-7, which is incorporated by reference), gene expression profiles can be used for class prediction and discriminating AML from ALL samples. However, for the analysis of acute leukemias the selection of the two different subgroups was performed using exclusively morphologic-phenotypical criteria. This was only descriptive and did not provide deeper insights into the pathogenesis or the underlying biology of the leukemia. The approach reproduces only very basic knowledge of cytomorphology and intends to differentiate classes. However, the data generated via such an approach is generally not sufficient to predict prognostically relevant cytogenetic aberrations.

### SUMMARY OF THE INVENTION

The present invention relates to rapid, cost effective, and reliable approaches to detecting and classifying leukemia. Aside from providing diagnostic information to patients, these classifications can also assist in selecting appropriate therapies and in prognostication. In some embodiments, these methods include profiling the expression of selected populations of genes using real-time PCR analysis, oligonucleotide arrays, or the like. In addition to methods, the invention also provides, e.g., related kits and systems.

In one aspect, the invention provides a method of classifying an acute myeloid leukemia (AML) cell. The method includes detecting an expression level of at least one set of genes in or derived from at least one target AML cell. In some embodiments, the target AML cell comprises an intermediate karyotype. The set of genes in or derived from the target AML cell generally comprises at least about 10, 1100, 1000, 10000, or more members. Typically, the target AML cell is obtained from a subject. The method also includes correlating a detected differential expression of one or more genes selected from the markers listed in one or more of Tables 1-13 relative to a corresponding expression of the genes in or derived from at least one reference AML cell having a reciprocal translocation (e.g., a t(15;17), t(8;21), inv(16), t(11q23), inv(3), etc.) with the target AML cell having a CEBPA mutation; correlating a detected substantially identical expression of one or more genes selected from the markers listed in one or more of Tables 1-13 relative to a corresponding expression of the genes in or derived from at least one reference AML cell having a CEBPA mutation with the target AML cell having the CEBPA mutation; correlating a detected differential expression of one or more genes selected from the markers listed in one or more of Tables 1-13 relative to a corresponding expression of the genes in or derived from at least one reference AML cell having a CEBPA mutation with the target AML cell having a reciprocal translocation; or correlating a detected substantially identical expression of one or more genes selected from the markers listed in one or more of Tables 1-13 relative to a corresponding expression of the genes in or derived from at least one reference AML cell having a reciprocal translocation with the target AML cell having the reciprocal translocation, thereby classifying the AML cell. In certain embodiments, the detected differential expression of the genes comprises at least about a 5% difference, whereas the detected substantially identical expression of the genes comprises less than about a 5% difference.

In some embodiments, the method also includes correlating a detected differential expression of one or more genes of the target AML cell relative to a corresponding expression of the genes in or derived from a reference AML cell with t(15;17), t(8;21), inv(16), or 11q23/MLL with the target AML cell being a target AML cell with t(8;16); or correlating a detected substantially identical expression of one or more genes of the target AML cell relative to a corresponding expression of the genes in or derived from a reference AML cell with t(8;16) with the target AML cell being a target AML cell with t(8;16), thereby detecting AML with t(8;16). In some embodiments, the detected differential or substantially identical expression comprises one or more markers selected from Table 1. In certain embodiments, the expression level comprises a higher expression of one or more markers selected from the group consisting of: a BCOR gene, a COXB5 gene, a CDK10 gene, a FLI1 gene, a HNRPA2B1 gene, a NSEP1 gene, a PDIP38 gene, a RAD50 gene, a SUPT5H gene, a TLR2 gene, a USP33 gene, a CEBP beta gene, a DDB2 gene, a HIST1H3D gene, a NSAP1 gene, a PTPNS1 gene, a RAN gene, a USP4 gene, a TRIM8 gene, and a ZNF278 gene in the target AML cell relative to a corresponding expression of the genes in or derived from the reference AML cell with t(15;17), t(8;21), inv(16), or 11q23/MLL. In certain embodiments, the expression level comprises a lower expression of one or more markers selected from the group consisting of: an ERG gene, a GATA2 gene, a NCOR2 gene, an RPS20 gene, a KIT gene, and an MBD2 gene in the target AML cell relative to a corresponding expression of the genes in or derived from the reference AML cell with t(15;17), t(8;21), inv(16), or 11q23/MLL. Typically, the detected differential expression of the genes comprises at least about a 5% difference, whereas the detected substantially identical expression of the genes comprises less than about a 5% difference.

To further illustrate, the detected differential or substantially identical expression expression comprises one or more of the markers listed in Table 3 and/or Table 4 when the reciprocal translocation comprises a t(11q23) in certain embodiments. In some embodiments, the detected differential or substantially identical expression expression comprises one or more of the markers listed in Table 5 and/or Table 6 when the reciprocal translocation comprises an inv(16). In certain embodiments, the detected differential or substantially identical expression expression comprises one or more of the markers listed in Table 7 and/or Table 8 when the reciprocal translocation comprises an inv(3). In some embodiments, the detected differential or substantially identical expression expression comprises one or more of the markers listed in Table 9 and/or Table 10 when the reciprocal translocation comprises a t(8;21). In certain embodiments, the detected differential or substantially identical expression expression comprises one or more of the markers listed in Table 11 and/or Table 12 when the reciprocal translocation comprises a t(15;17).

In some embodiments, the method includes further classifying two different subgroups of CEBPA mutations (group A and group B). Group A is defined as having mutations in the TAD2 domain of CEBPA and a high percentage of FLT3-LM in addition. In contrast, group B has mutations that lead to an N-terminal stop mutation and has only a low percentage of FLT3-LM. Accordingly, in some embodiments, the method includes correlating a detected higher expression of an MPO gene from the target AML cell having a CEBPA mutation, and/or a detected lower expression of one or more of: a HOXA3 gene, a HOXA7 gene, a HOXA9 gene, a HOXB4 gene, a HOXB6 gene, or a PBX3 gene from the target AML cell having the CEBPA mutation, relative to at least one reference AML cell lacking the CEBPA mutation with the target AML being a Group A AML cell; or correlating a detected lower expression of an MPO gene from the target AML cell having a CEBPA mutation, and/or a detected higher expression of one or more of: a HOXA3 gene, a HOXA7 gene, a HOXA9 gene, a HOXB4 gene, a HOXB6 gene, and a PBX3 gene from the target AML cell having the CEBPA mutation, relative to at least one reference AML cell lacking the CEBPA mutation with the target AML being a Group B AML cell (see, TABLE 2).

Expression levels are detected using essentially any gene expression profiling technique. In some embodiments, for example, the expression level is detected using an array, a robotics system, and/or a microfluidic device. In certain embodiments, the expression level of the set of genes is detected by amplifying nucleic acid sequences associated with the genes to produce amplicons and detecting the amplicons. In these embodiments, the amplicons are generally detected using a process that comprises one or more of: hybridizing the amplicons to an oligonucleotide array, digesting the amplicons with a restriction enzyme, or real-time polymerase chain reaction (PCR) analysis. In certain embodiments, the expression level of the set of genes is detected by, e.g., measuring quantities of transcribed polynucleotides (e.g., mRNAs, cDNAs, etc.) or portions thereof expressed or derived from the genes. In some embodiments, the expression level is detected by, e.g., contacting polynucleotides or polypeptides expressed from the genes with compounds (e.g., aptamers, antibodies or fragments thereof, etc.) that specifically bind the polynucleotides or polypeptides.

Essentially any method of detecting the mutational status of the genes is optionally utilized. In some embodiments, for example, the mutational status is detected by sequencing the genes. To further illustrate, the mutational status is optionally detected by amplifying nucleic acid sequences associated with the genes to produce amplicons and detecting the amplicons. In these embodiments, the amplicons are generally detected using a process that comprises one or more of, e.g., hybridizing the amplicons to an oligonucleotide array, digesting the amplicons with a restriction enzyme, real-time polymerase chain reaction (PCR) analysis, or the like.

In another aspect, the invention provides a method of producing a reference data bank for classifying AML cells. The method includes (a) compiling a gene expression profile of a patient sample by detecting the expression level of one or more genes of at least one AML cell, which genes are selected from the markers listed in one or more of Tables 1-42, and (b) classifying the gene expression profile using a machine learning algorithm.

In another aspect, the invention provides a kit that includes one or more probes that correspond to at least portions of genes or expression products thereof, which genes are selected from the markers listed in one or more of Tables 1-42. In some embodiments, at least one solid support comprises the probes. Optionally, the kit also includes one or more additional reagents to perform real-time PCR analyses. In addition, the kit also includes instructions for correlating detected expression levels of polynucleotides and/or polypeptides in at least one target cell from a subject, which polynucleotides and/or polypeptides are targets of one or more of the probes, with the target cell being an AML cell having a CEBPA mutation or a reciprocal translocation.

In another aspect, the invention provides a system that includes one or more probes that correspond to at least portions of genes or expression products thereof, which genes are selected from the markers listed in one or more of Tables 1-42. In some embodiments, at least one solid support comprises the probes. In certain embodiments, the system includes one or more additional reagents and/or components to perform real-time PCR analyses. The system also includes at least one reference data bank for correlating detected expression levels of polynucleotides and/or polypeptides in at least one target cell from a subject, which polynucleotides and/or polypeptides are targets of one or more of the probes, with the target cell being an AML cell having a CEBPA mutation or a reciprocal translocation. The reference data bank is generally produced by, e.g., (a) compiling a gene expression profile of a patient sample by detecting the expression level at least one of the genes, and (b) classifying the gene expression profile using a machine learning algorithm. The machine learning algorithm is generally selected from, e.g., a weighted voting algorithm, a K-nearest neighbors algorithm, a decision tree induction algorithm, a support vector machine, a feed-forward neural network, etc.

In one aspect, the invention provides a method of aiding in a leukemia prognosis for a subject. The method includes detecting an expression level of at least one set of genes in or derived from at least one target acute myeloid leukemia (AML) cell from the subject. In some embodiments, the set of genes is selected from one or more of: Tables 15-17. The method also includes correlating a detected a higher expression of an MPO gene and/or an ATBF1 gene in the target AML cell relative to a corresponding expression of the genes in or derived from an AML cell from a member of an unfavorable group with the subject having a probable overall survival rate at three years of about 55% or more; or correlating a detected a higher expression of one or more of: an ETS2 gene, a RUNX1 gene, a TCF4 gene, a FOXC1 gene, a SFRS1 gene, a TPD52 gene, a NRIP1 gene, a TFPI gene, a UBL1 gene, an REC8L1 gene, an HSF2 gene, or an ETS2 gene in the target AML cell relative to a corresponding expression of the genes in or derived from an AML cell from a member of a favorable group with the subject having a probable overall survival rate at three years of about 25% or less, thereby aiding in the leukemia prognosis for the subject. Typically, the higher expression of the genes in the target AML cell is at least 5% greater than the corresponding expression of the genes in or derived from the AML cell from the member of the unfavorable group or the favorable group. The unfavorable group generally comprises a probable overall survival rate at three years of about 25% or less, whereas the favorable group typically comprises a probable overall survival rate at three years of about 55% or more.

In another aspect, the invention provides a method of producing a reference data bank for aiding in leukemia prognostication. The method includes (a) compiling a gene expression profile of a patient sample by determining the expression level at least one marker selected from: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker. The method also includes (b) classifying the gene expression profile using a machine learning algorithm.

In one aspect, the invention provides a method of identifying an acute myeloid leukemia (AML) cell comprising trisomy 8. The method includes (a) detecting an expression level of at least one set of genes in or derived from at least one target human AML cell. The target human AML cell is generally obtained from a subject. In some embodiments, the set of genes in or derived from the target human AML cell comprises at least about 10, 100, 1000, 10000, or more members. The method also includes (b) correlating a detected differential expression of one or more genes of chromosome 8 of the target human AML cell relative to a corresponding expression of the genes in or derived from a human AML cell lacking trisomy 8 with the target human AML cell comprising trisomy 8; or (c) correlating a detected substantially identical expression of one or more genes of the target human AML cell relative to a corresponding expression of the genes in or derived from a human AML cell comprising trisomy 8 with the target human AML cell comprising trisomy 8, thereby identifying the AML cell comprising trisomy 8. Typically, the human AML cell lacking trisomy 8 comprises one or more of: a normal karyotype, a complex aberrant karyotype, t(15;17), inv(16), t(8;21), 11q23/MLL, or another abnormality. In certain embodiments, the detected differential expression of the genes comprises a higher mean expression of a substantial number of the genes of chromosome 8 of the target human AML cell relative to the corresponding expression of the genes in or derived from the human AML cell lacking trisomy 8. Typically, the detected differential expression of the genes comprises at least about a 5% difference, whereas the detected substantially identical expression of the genes comprises less than about a 5% difference.

The methods described herein include detecting the expression levels various sets of genes. In some embodiments, for example, the detected differential or substantially identical expression comprises one or more markers selected from Table 19. In some embodiments, the human AML cell lacking trisomy 8 comprises t(8;21) and the detected differential or substantially identical expression comprises one or more markers selected from Table 21. In certain embodiments, the human AML cell lacking trisomy 8 comprises t(15;17) and the detected differential or substantially identical expression comprises one or more markers selected from Table 23. In some embodiments, the human AML cell lacking trisomy 8 comprises inv(16) and the detected differential or substantially identical expression comprises one or more markers selected from Table 25. In certain embodiments, the human AML cell lacking trisomy 8 comprises 11q23/MLL and the detected differential or substantially identical expression comprises one or more markers selected from Table 27. In some embodiments, the human AML cell lacking trisomy 8 comprises a normal karyotype and the detected differential or substantially identical expression comprises one or more markers selected from Table 29. In certain embodiments, the human AML cell lacking trisomy 8 comprises at least one other abnormality and the detected differential or substantially identical expression comprises one or more markers selected from Table 31. In certain embodiments, the human AML cell lacking trisomy 8 comprises a complex aberrant karyotype and the detected differential or substantially identical expression comprises one or more markers selected from Table 33.

To further illustrate, (b) comprises correlating a detected differential expression of one or more genes of chromosome 8 of the target human AML cell relative to the corresponding expression of the genes in or derived from the human AML cell lacking trisomy 8 with the target human AML cell comprising trisomy 8, and (c) comprises correlating a detected substantially identical expression of one or more genes of chromosome 8 of the target human AML cell relative to a corresponding expression of the genes in or derived from a human AML cell comprising trisomy 8 with the target human AML cell comprising trisomy 8 in certain embodiments. In some of these embodiments, the detected differential or substantially identical expression comprises one or more markers selected from Table 20. In certain of these embodiments, the human AML cell lacking trisomy 8 comprises t(8;21) and the detected differential or substantially identical expression comprises one or more markers selected from Table 22. In some of these embodiments, the human AML cell lacking trisomy 8 comprises t(15;17) and the detected differential or substantially identical expression comprises one or more markers selected from Table 24. In certain of these embodiments, the human AML cell lacking trisomy 8 comprises inv(16) and the detected differential or substantially identical expression comprises one or more markers selected from Table 26. In some of these embodiments, the human AML cell lacking trisomy 8 comprises 11q23/MLL and the detected differential or substantially identical expression comprises one or more markers selected from Table 28. In certain of these embodiments, wherein the human AML cell lacking trisomy 8 comprises a normal karyotype and the detected differential or substantially identical expression comprises one or more markers selected from Table 30. In some of these embodiments, the human AML cell lacking trisomy 8 comprises at least one other abnormality and the detected differential or substantially identical expression comprises one or more markers selected from Table 32. In certain of these embodiments, the human AML cell lacking trisomy 8 comprises a complex aberrant karyotype and the detected differential or substantially identical expression comprises one or more markers selected from Table 34.

In another aspect, the invention provides a kit that includes one or more markers or portions thereof selected from the group consisting of: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker. In some embodiments, at least one solid support comprises the markers or the portions thereof. In certain embodiments, the kit includes one or more additional reagents to perform real-time PCR analyses. The kit also includes instructions for correlating detected expression levels of polynucleotides and/or polypeptides in at least one target AML cell from a subject, which polynucleotides and/or polypeptides correspond to one or more of the markers, with a probable overall survival rate for the subject. Optionally, the kit includes a reference (e.g., a sample, a data bank, etc.) corresponding to a favorable group and/or an unfavorable group.

In another aspect, the invention provides a system that includes one or more markers or portions thereof selected from the group consisting of: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker.

In some embodiments, the detected differential expression of the genes comprises a higher expression (e.g., positive fold change, etc.) of a FLT3 gene of the target cell relative to the corresponding expression of the FLT3 gene in or derived from the MDS cell. In certain embodiments, the detected differential expression of the genes comprises a lower expression (e.g., negative fold change, etc.) of a FLT3 gene of the target cell relative to the corresponding expression of the FLT3 gene in or derived from the AML cell. In some embodiments, the detected substantially identical expression of the genes comprises a substantially identical expression of a FLT3 gene of the target cell relative to the corresponding expression of the FLT3 gene in or derived from the AML cell. See, e.g., Table 35, where the r values refer to MDS and AML blasts in comparison to percentage; e.g., most genes exhibit higher expression in MDS, but FTL3 is expressed higher in AML.

In certain embodiments, the detected differential expression of the genes comprises a higher expression of one or more of: ANXA3, ARG1, CAMP, CD24, CEACAM1, CEACAM6, CEACAM8, CRISP3, KIAA0922, LCN2, MMP9, or, STOM of the target cell relative to the corresponding expression of the genes in or derived from the AML cell. In some embodiments, the detected differential expression of the genes comprises a lower expression of one or more of: ANXA3, ARG1, CAMP, CD24, CEACAM1, CEACAM6, CEACAM8, CRISP3, KIAA0922, LCN2, MMP9, or STOM of the target cell relative to the corresponding expression of the genes in or derived from the MDS cell. In certain embodiments, the detected substantially identical expression of the genes comprises a substantially identical expression of one or more of: ANXA3, ARG1, CAMP, CD24, CEACAM1, CEACAM6, CEACAM8, CRISP3, KIAA0922, LCN2, MMP9, or STOM of the target cell relative to the corresponding expression of the genes in or derived from the MDS cell. See, e.g., Tables 35 and 36.

In certain embodiments, the method includes correlating a detected differential expression of one or more genes of the target cell, which genes are selected from the markers listed in Table 37, relative to a corresponding expression of the genes in or derived from an AML cell having a normal karyotype or an MDS cell having a normal karyotype with the target cell being an AML cell having a complex aberrant karyotype or an MDS cell having a complex aberrant karyotype. In some embodiments, the method includes correlating a detected substantially identical expression of one or more genes of the target cell, which genes are selected from the markers listed in Table 37, relative to a corresponding expression of the genes in or derived from an AML cell having a normal karyotype or an MDS cell having a normal karyotype with the target cell being an AML cell having a normal karyotype or an MDS cell having a normal karyotype. In certain embodiments, the method includes correlating a detected differential expression of one or more genes of the target cell, which genes are selected from the markers listed in Table 37, relative to a corresponding expression of the genes in or derived from an AML cell having a complex aberrant karyotype or an MDS cell having a complex aberrant karyotype with the target cell being an AML cell having a normal karyotype or an MDS cell having a normal karyotype. In some embodiments, the method includes correlating a detected substantially identical expression of one or more genes of the target cell, which genes are selected from the markers listed in Table 37, relative to a corresponding expression of the genes in or derived from an AML cell having a complex aberrant karyotype or an MDS cell having a complex aberrant karyotype with the target cell being an AML cell having a complex aberrant karyotype or an MDS cell having a complex aberrant karyotype.

In one aspect, the invention provides a method of subclassifying acute myeloid leukemia with normal karyotype (AML-NK). The method includes detecting an expression level of at least one set of genes in or derived from at least one target AML-NK cell. In addition, the method also includes correlating: a detected higher expression of one or more genes selected from the group listed in Table 38 and/or a detected lower expression of one or more genes selected from the group listed in Table 39 of the target AML-NK cell relative to a corresponding expression of the genes in or derived from a Group B AML-NK cell with the target AML-NK cell being a Group A AML-NK cell; or a detected lower expression of one or more genes selected from the group listed in Table 38 and/or a detected higher expression of one or more genes selected from the group listed in Table 39 of the target AML-NK cell relative to a corresponding expression of the genes in or derived from a Group A AML-NK cell with the target AML-NK cell being a Group B AML-NK cell. The set of genes in or derived from the target AML-NK cell typically comprises at least about 10, 100, 1000, 10000, or more members. Further, the set of genes is in the form of transcribed polynucleotides (e.g., mRNAs, cDNAs, etc.) or portions thereof in some embodiments. The higher expression and/or the lower expression of the genes generally comprises at least about a 5% difference. The target AML-NK cell is generally obtained from a subject. Moreover, a subclassification of the target AML-NK cell in Group B typically correlates with a better event-free survival rate and/or overall survival rate for the subject than a subclassification of the target AML-NK cell in Group A.

In one aspect, the invention provides a method of identifying a cell with a 5q deletion ((del)5q). The method includes detecting an expression level of at least one set of genes in or derived from at least one target human cell. In some embodiments, the target human cell comprises an acute myeloid leukemia (AML) cell or a myelodysplastic syndrome (MDS) cell. The target human cell is generally obtained from a subject. Typically, the set of genes in or derived from the target human cell comprises at least about 10, 100, 1000, 10000, or more members. The method also includes correlating a detected differential expression of one or more genes of at least chromosome 5 of the target human cell relative to a corresponding expression of the genes in or derived from a human cell lacking a (del)5q (e.g., a myeloid cell, etc.) with the target human cell comprising a (del)5q; or correlating a detected substantially identical expression of one or more genes of at least chromosome 5 of the target human cell relative to a corresponding expression of the genes in or derived from a human cell having a (del)5q (e.g., a myeloid cell, etc.) with the target human cell comprising a (del)5q, thereby identifying the cell with the (del)5q. In some embodiments, the method include correlating the detected differential expression of the genes with the target human cell being an AML cell with a normal karyotype (AML-NK), an MDS cell with a normal karyotype (MDS-NK), or an MDS cell with a complex aberrant karyotype. Typically, the detected differential expression of the genes comprises at least about a 5% difference, whereas the detected substantially identical expression of the genes typically comprises less than about a 5% difference.

In certain embodiments, the detected differential expression of the genes comprises a lower mean expression of a substantial number of the genes located on a long arm of chromosome 5 of the target human cell relative to the corresponding expression of the genes in or derived from the human cell lacking the (del)5q. In some embodiments, the detected differential expression comprises an expression of one or more genes selected from the group consisting of: POLE, RAD21, RAD23B, ZNF75A, AF020591, MLLT3, HOXB6, UPF2, TINP1, RPL12, RPL14, RPL15, GMNN, CSPG6, PFDN1, HINT1, STK24, APP, and CAMLG. In some embodiments, the detected differential expression of the genes comprises a lower expression of one or more of the genes listed in Table 41 (e.g., CSNK1A1, DAMS, HDAC3, PFDN1, CNOT8, etc.) of the target human cell relative to the corresponding expression of the genes in or derived from the human cell lacking the (del)5q. Table 41 lists genes located on the long (q) arm of chromosome 5 that are downregulated or lower expressed in cases with (del)5q compared to cases without (del)5q. In certain embodiments, the detected differential expression of the genes comprises: a higher expression of one or more of: RAD21, RAD23B, GMMN, CSPG6, APP, POLE, STK24, STAG2, H1F0, PTPN11, or TAF2 of the target human cell relative to the corresponding expression of the genes in or derived from the human cell lacking the (del)5q; and/or a lower expression of one or more of: ACTA2, RPL12, DF, UBE2D2, EEF1A1, IGBP1, PPP2CA, EIF2S3, or NACA of the target human cell relative to the corresponding expression of the genes in or derived from the human cell lacking the (del)5q.

The system also includes at least one reference data bank for correlating detected expression levels of polynucleotides and/or polypeptides in target AML cells, which polynucleotides and/or polypeptides correspond to one or more of the markers, with a probable overall survival rate for a subject. Typically, the reference data bank is produced by: (a) compiling a gene expression profile of a patient sample by determining the expression level at least one of the markers, and (b) classifying the gene expression profile using a machine learning algorithm. The machine learning algorithm is typically selected from, e.g., a weighted voting algorithm, a K-nearest neighbors algorithm, a decision tree induction algorithm, a support vector machine, a feed-forward neural network, or the like.

### DETAILED DESCRIPTION

### Definitions

Before describing the present invention in detail, it is to be understood that this invention is not limited to particular embodiments. It is also to be understood that the terminology used herein is for the purpose of describing particular embodiments only, and is not intended to be limiting. Units, prefixes, and symbols are denoted in the forms suggested by the International System of Units (SI), unless specified otherwise. Numeric ranges are inclusive of the numbers defining the range. As used in this specification and the appended claims, the singular forms “a”, “an” and “the” also include plural referents unless the context clearly dictates otherwise. To illustrate, reference to “a cell” includes two or more cells. Further, unless defined otherwise, all technical and scientific terms used herein have the same meaning as commonly understood by one of ordinary skill in the art to which the invention pertains. The terms defined below, and grammatical variants thereof, are more fully defined by reference to the specification in its entirety.

A “5q deletion” or “(del)5q” refers to deletions (e.g., acquired interstitial deletions) of the long arm of a human chromosome 5.

“11q23/MLL” refers to acute myeloid leukemia with the 11q23 rearrangement of the human MLL gene according to the World Health Organization (WHO) classification of haematological malignancies.

An “antibody” refers to a polypeptide substantially encoded by at least one immunoglobulin gene or fragments of at least one immunoglobulin gene, which can participate in specific binding with a ligand. The term “antibody” includes polyclonal and monoclonal antibodies and biologically active fragments thereof including among other possibilities “univalent” antibodies (Glennie et al. (1982) Nature 295:712); Fab proteins including Fab′ and F(ab′)₂ fragments whether covalently or non-covalently aggregated; light or heavy chains alone, typically variable heavy and light chain regions (V_{H} and V_{L} regions), and more typically including the hypervariable regions (otherwise known as the complementarity determining regions (CDRs) of the V_{H} and V_{L} regions); F_{c} proteins; “hybrid” antibodies capable of binding more than one antigen; constant-variable region chimeras; “composite” immunoglobulins with heavy and light chains of different origins; “altered” antibodies with improved specificity and other characteristics as prepared by standard recombinant techniques, by mutagenic techniques, or other directed evolutionary techniques known in the art. Derivatives of antibodies include scFvs, chimeric and humanized antibodies. See, e.g., Harlow and Lane, Antibodies a laboratory manual, CSH Press (1988), which is incorporated by reference. For the detection of polypeptides using antibodies or fragments thereof, there are a variety of methods known to a person skilled in the art, which are optionally utilized. Examples include immunoprecipitations, Western blottings, Enzyme-linked immuno sorbent assays (ELISA), radioimmunoassays (RIA), dissociation-enhanced lanthanide fluoro immuno assays (DELFIA), scintillation proximity assays (SPA). To facilitate detection, an antibody is typically labeled by one or more of the labels described herein or otherwise known to persons skilled in the art.

In general, an “array” or “microarray” refers to a linear or two- or three dimensional arrangement of preferably discrete nucleic acid or polypeptide probes which comprises an intentionally created collection of nucleic acid or polypeptide probes of any length spotted onto a substrate/solid support. The person skilled in the art knows a collection of nucleic acids or polypeptide spotted onto a substrate/solid support also under the term “array”. As also known to the person skilled in the art, a microarray usually refers to a miniaturized array arrangement, with the probes being attached to a density of at least about 10, 20, 50, 100 nucleic acid molecules referring to different or the same genes per cm². Furthermore, where appropriate an array can be referred to as “gene chip”. The array itself can have different formats, e.g., libraries of soluble probes or libraries of probes tethered to resin beads, silica chips, or other solid supports.

“Complementary” and “complementarity”, respectively, can be described by the percentage, i.e., proportion, of nucleotides that can form base pairs between two polynucleotide strands or within a specific region or domain of the two strands. Generally, complementary nucleotides are, according to the base pairing rules, adenine and thymine (or adenine and uracil), and cytosine and guanine. Complementarity may be partial, in which only some of the nucleic acids' bases are matched according to the base pairing rules. Or, there may be a complete or total complementarity between the nucleic acids. The degree of complementarity between nucleic acid strands has effects on the efficiency and strength of hybridization between nucleic acid strands.

Two nucleic acid strands are considered to be 100% complementary to each other over a defined length if in a defined region all adenines of a first strand can pair with a thymine (or an uracil) of a second strand, all guanines of a first strand can pair with a cytosine of a second strand, all thymine (or uracils) of a first strand can pair with an adenine of a second strand, and all cytosines of a first strand can pair with a guanine of a second strand, and vice versa. According to the present invention, the degree of complementarity is determined over a stretch of about 20 or 25 nucleotides, i.e., a 60% complementarity means that within a region of 20 nucleotides of two nucleic acid strands 12 nucleotides of the first strand can base pair with 12 nucleotides of the second strand according to the above base pairing rules, either as a stretch of 12 contiguous nucleotides or interspersed by non-pairing nucleotides, when the two strands are attached to each other over the region of 20 nucleotides. The degree of complementarity can range from at least about 50% to full, i.e., 100% complementarity. Two single nucleic acid strands are said to be “substantially complementary” when they are at least about 80% complementary, and more typically about 90% complementary or higher. For carrying out the methods of present invention substantial complementarity is generally utilized.

Two nucleic acids “correspond” when they have substantially identical or complementary sequences, when one nucleic acid is a subsequence of the other, or when one sequence is derived naturally or artificially from the other.

The term “differential gene expression” refers to a gene or set of genes whose expression is activated to a higher or lower level in a subject suffering from a disease, (e.g., cancer) relative to its expression in a normal or control subject. Differential gene expression can also occur between different types or subtypes of diseased cells. The term also includes genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between, e.g., normal subjects and subjects suffering from a disease, various stages of the same disease, different types or subtypes of diseased cells, etc. Differential expression includes both quantitative, as well as qualitative, differences in the temporal or cellular expression pattern in a gene or its expression products among, for example, normal and diseased cells, or among cells which have undergone different disease events or disease stages. In certain embodiments, “differential gene expression” is considered to be present when there is at least an about two-fold, typically at least about four-fold, more typically at least about six-fold, most typically at least about ten-fold difference between, e.g., the expression of a given gene in normal and diseased subjects, in various stages of disease development in a diseased subject, different types or subtypes of diseased cells, etc.

The term “expression” refers to the process by which mRNA or a polypeptide is produced based on the nucleic acid sequence of a gene, i.e., “expression” also includes the formation of mRNA in the process of transcription. The term “determining the expression level” refers to the determination of the level of expression of one or more markers.

The term “genotype” refers to a description of the alleles of a gene or genes contained in an individual or a sample. As used herein, no distinction is made between the genotype of an individual and the genotype of a sample originating from the individual. Although, typically, a genotype is determined from samples of diploid cells, a genotype can be determined from a sample of haploid cells, such as a sperm cell.

The term “gene” refers to a nucleic acid sequence encoding a gene product. The gene optionally comprises sequence information required for expression of the gene (e.g., promoters, enhancers, etc.).

The term “gene expression data” refers to one or more sets of data that contain information regarding different aspects of gene expression. The data set optionally includes information regarding: the presence of target-transcripts in cell or cell-derived samples; the relative and absolute abundance levels of target transcripts; the ability of various treatments to induce expression of specific genes; and the ability of various treatments to change expression of specific genes to different levels.

Nucleic acids “hybridize” when they associate, typically in solution. Nucleic acids hybridize due to a variety of well-characterized physico-chemical forces, such as hydrogen bonding, solvent exclusion, base stacking and the like. In certain embodiments, hybridization occurs under conventional hybridization conditions, such as under stringent conditions as described, for example, in Sambrook et al., in “Molecular Cloning: A Laboratory Manual” (1989), Eds. J. Sambrook, E. F. Fritsch and T. Maniatis, Cold Spring Harbour Laboratory Press, Cold Spring Harbour, N.Y., which is incorporated by reference. Such conditions are, for example, hybridization in 6×SSC, pH 7.0/0.1% SDS at about 45° C. for 18-23 hours, followed by a washing step with 2×SSC/1% SDS at 50° C. In order to select the stringency, the salt concentration in the washing step can, for example, be chosen between 2×SSC/0.1% SDS at room temperature for low stringency and 0.2×SSC/0.1% SDS at 50° C. for high stringency. In addition, the temperature of the washing step can be varied between room temperature (ca. 22° C.), for low stringency, and 65° C. to 70° C. for high stringency. Also contemplated are polynucleotides that hybridize at lower stringency hybridization conditions. Changes in the stringency of hybridization and signal detection are primarily accomplished through the manipulation of, e.g., formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 mg/mL salmon sperm blocking DNA, followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. The inclusion of specific blocking reagents may require modification of the hybridization conditions described herein, due to problems with compatibility. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, (1997), which are each incorporated by reference. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis; labeling, detection and quantification of DNA and RNA, including oligonucleotides. Both Hames and Higgins 1 and 2 are incorporated by reference.

“inv(3)” refers to an inversion of human chromosome 3.

“inv(16)” refers to AML with inversion 16 according to the WHO classification of haematological malignancies.

A “label” refers to a moiety attached (covalently or non-covalently), or capable of being attached, to a molecule (e.g., a polynucleotide, a polypeptide, etc.), which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), non-fluorescent labels, colorimetric labels, chemiluminescent labels, bioluminescent labels, radioactive labels (such as ³H, ³⁵S, ³²P, ¹²⁵I, ⁵⁷Co or ¹⁴C), mass-modifying groups, antibodies, antigens, biotin, haptens, digoxigenin, enzymes (including, e.g., peroxidase, phosphatase, etc.), and the like. To further illustrate, fluorescent labels may include dyes that are negatively charged, such as dyes of the fluorescein family, or dyes that are neutral in charge, such as dyes of the rhodamine family, or dyes that are positively charged, such as dyes of the cyanine family. Dyes of the fluorescein family include, e.g., FAM, HEX, TET, JOE, NAN and ZOE. Dyes of the rhodamine family include, e.g., Texas Red, ROX, R110, R6G, and TAMRA. FAM, HEX, TET, JOE, NAN, ZOE, ROX, R110, R6G, and TAMRA are commercially available from, e.g., Perkin-Elmer, Inc. (Wellesley, Mass., USA), and Texas Red is commercially available from, e.g., Molecular Probes, Inc. (Eugene, Oreg., USA). Dyes of the cyanine family include, e.g., Cy2, Cy3, Cy3.5, Cy5, Cy5.5, and Cy7, and are commercially available from, e.g., Amersham Biosciences Corp. (Piscataway, N.J., USA). Suitable methods include the direct labeling (incorporation) method, an amino-modified (amino-allyl) nucleotide method (available e.g. from Ambion, Inc. (Austin, Tex., USA), and the primer tagging method (DNA dendrimer labeling, as kit available e.g. from Genisphere, Inc. (Hatfield, Pa., USA)). In some embodiments, biotin or biotinylated nucleotides are used for labeling, with the latter generally being directly incorporated into, e.g., the cRNA polynucleotide by in vitro transcription.

The term “lower expression” refers an expression level of one or more markers from a target that is less than a corresponding expression level of the markers in a reference. In certain embodiments, “lower expression” is assigned to all by numbers and Affymetrix Id. definable polynucleotides the t-values and fold change (fc) values of which are negative. Similarly, the term “higher expression” refers an expression level of one or more markers from a target that is more than a corresponding expression level of the markers in a reference. In some embodiments, “higher expression” is assigned to all by numbers and Affymetrix Id. definable polynucleotides the t-values and fold change (fc) values of which are positive.

A “machine learning algorithm” refers to a computational-based prediction methodology, also known to persons skilled in the art as a “classifier”, employed for characterizing a gene expression profile. The signals corresponding to certain expression levels, which are obtained by, e.g., microarray-based hybridization assays, are typically subjected to the algorithm in order to classify the expression profile. Supervised learning generally involves “training” a classifier to recognize the distinctions among classes and then “testing” the accuracy of the classifier on an independent test set. For new, unknown samples the classifier can be used to predict the class in which the samples belong.

The term “marker” refers to a genetically controlled difference that can be used in the genetic analysis of a test or target versus a control or reference sample for the purpose of assigning the sample to a defined genotype or phenotype. In certain embodiments, for example, “markers” refer to genes, polynucleotides, polypeptides, or fragments or portions thereof that are differentially expressed in, e.g., different leukemia types and/or subtypes. The markers can be defined by their gene symbol name, their encoded protein name, their transcript identification number (cluster identification number), the data base accession number, public accession number and/or GenBank identifier. Markers can also be defined by their Affymetrix identification number, chromosomal location, UniGene accession number and cluster type, and/or LocusLink accession number. The Affymetrix identification number (affy id) is accessible for anyone and the person skilled in the art by entering the “gene expression omnibus” internet page of the National Center for Biotechnology Information (NCBI) on the world wide web at ncbi.nlm.nih.gov/geo/ as of Nov. 4, 2004. In particular, the affy id's of the polynucleotides used for certain embodiments of the methods described herein are derived from the so-called human genome U133 chip (Affymetrix, Inc., Santa Clara, Calif., USA). The sequence data of each identification number can be viewed on the world wide web at, e.g., ncbi.nlm.nih.gov/projects/geo/ as of Nov. 4, 2004 using the accession number GPL96 for U133A annotational data and accession number GPL97 for U133B annotational data. In some embodiments, the expression level of a marker is determined by the determining the expression of its corresponding polynucleotide.

The term “normal karyotype” refers to a state of those cells lacking any visible karyotype abnormality detectable with chromosome banding analysis.

The term “nucleic acid” refers to a polymer of monomers that can be corresponded to a ribose nucleic acid (RNA) or deoxyribose nucleic acid (DNA) polymer, or analog thereof. This includes polymers of nucleotides such as RNA and DNA, as well as modified forms thereof, peptide nucleic acids (PNAs), locked nucleic acids (LNA™s), and the like. In certain applications, the nucleic acid can be a polymer that includes multiple monomer types, e.g., both RNA and DNA subunits. A nucleic acid can be or include, e.g., a chromosome or chromosomal segment, a vector (e.g., an expression vector), an expression cassette, a naked DNA or RNA polymer, the product of a polymerase chain reaction (PCR) or other nucleic acid amplification reaction, an oligonucleotide, a probe, a primers, etc. A nucleic acid can be e.g., single-stranded or double-stranded. Unless otherwise indicated, a particular nucleic acid sequence optionally comprises or encodes complementary sequences, in addition to any sequence explicitly indicated.

Oligonucleotides (e.g., probes, primers, etc.) of a defined sequence may be produced by techniques known to those of ordinary skill in the art, such as by chemical or biochemical synthesis, and by in vitro or in vivo expression from recombinant nucleic acid molecules, e.g., bacterial or retroviral vectors.

Oligonucleotides which are primer and/or probe sequences, as described below, may comprise DNA, RNA or nucleic acid analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are incorporated by reference. Such sequences can routinely be synthesized using a variety of techniques currently available. For example, a sequence of DNA can be synthesized using conventional nucleotide phosphoramidite chemistry and the instruments available from Applied Biosystems, Inc, (Foster City, Calif., USA); DuPont, (Wilmington, Del., USA); or Milligen, (Bedford, Mass., USA). Similarly, and when desirable, the sequences can be labeled using methodologies well known in the art such as described in U.S. Pat. Nos. 5,464,746; 5,424,414; and 4,948,882 all of which are incorporated by reference.

A nucleic acid, nucleotide, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide of the invention can contain one or more modified, non-standard, or derivatized base moieties, including, but not limited to, N⁶-methyl-adenine, N⁶-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611, 5,955,589, 5,844,106, 5,789,562, 5,750,343, 5,728,525, and 5,679,785, each of which is incorporated by reference.

Furthermore, a nucleic acid, nucleotide, polynucleotide or oligonucleotide can comprise one or more modified sugar moieties including, but not limited to, arabinose, 2-fluoroarabinose, xylulose, and hexose. A nucleic acid, nucleotide, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

The term “polynucleotide” refers to a DNA, in particular cDNA, or RNA, in particular a cRNA, or a portion thereof. In the case of RNA (or cDNA), the polynucleotide is formed upon transcription of a nucleotide sequence that is capable of expression. “Polynucleotide fragments” refer to fragments of between at least 8, such as 10, 12, 15 or 18 nucleotides and at least 50, such as 60, 80, 100, 200 or 300 nucleotides in length, or a complementary sequence thereto, e.g., representing a consecutive stretch of nucleotides of a gene, cDNA or mRNA. In some embodiments, polynucleotides also include any fragment (or complementary sequence thereto) of a sequence corresponding to or derived from any of the markers defined herein.

The term “primer” refers to an oligonucleotide having a hybridization specificity sufficient for the initiation of an enzymatic polymerization under predetermined conditions, for example in an amplification technique such as polymerase chain reaction (PCR), in a process of sequencing, in a method of reverse transcription and the like. The term “probe” refers to an oligonucleotide having a hybridization specificity sufficient for binding to a defined target sequence under predetermined conditions, for example in an amplification technique such as a 5′-nuclease reaction, in a hybridization-dependent detection method, such as a Southern or Northern blot, and the like. In certain embodiments, probes correspond at least in part to selected markers. Primers and probes may be used in a variety of ways and may be defined by the specific use. For example, a probe can be immobilized on a solid support by any appropriate means, including, but not limited to: by covalent bonding, by adsorption, by hydrophobic and/or electrostatic interaction, or by direct synthesis on a solid support (see in particular patent application WO 92/10092). A probe may be labeled by means of a label chosen, for example, from radioactive isotopes, enzymes, in particular enzymes capable of acting on a chromogenic, fluorescent or luminescent substrate (in particular a peroxidase or an alkaline phosphatase), chromophoric chemical compounds, chromogenic, fluorigenic or luminescent compounds, analogues of nucleotide bases, and ligands such as biotin. Illustrative fluorescent compounds include, for example, fluorescein, carboxyfluorescein, tetrachlorofluorescein, hexachlorofluorescein, Cy3, tetramethylrhodamine, Cy3.5, carboxy-x-rhodamine, Texas Red, Cy5, and Cy5.5. Illustrative luminescent compounds include, for example, luciferin and 2,3-dihydrophthalazinediones, such as luminol. Other suitable labels are described herein or are otherwise known to those of skill in the art.

Oligonucleotides (e.g., primers, probes, etc.), whether hybridization assay probes, amplification primers, or helper oligonucleotides, may be modified with chemical groups to enhance their performance or to facilitate the characterization of amplification products. For example, backbone-modified oligonucleotides such as those having phosphorothioate or methylphosphonate groups which render the oligonucleotides resistant to the nucleolytic activity of certain polymerases or to nuclease enzymes may allow the use of such enzymes in an amplification or other reaction. Another example of modification involves using non-nucleotide linkers (e.g., Arnold, et al., “Non-Nucleotide Linking Reagents for Nucleotide Probes”, EP 0 313 219, which is incorporated by reference) incorporated between nucleotides in the nucleic acid chain which do not interfere with hybridization or the elongation of the primer. Amplification oligonucleotides may also contain mixtures of the desired modified and natural nucleotides.

A “reference” in the context of gene expression profiling refers to a cell and/or genes in or derived from the cell (or data derived therefrom) relative to which a target is compared. In some embodiments, for example, the expression of one or more genes from a target cell is compared to a corresponding expression of the genes in or derived from a reference cell.

A “sample” refers to any biological material containing genetic information in the form of nucleic acids or proteins obtainable or obtained from one or more subjects or individuals. In some embodiments, samples are derived from subjects having leukemia, e.g., AML. Exemplary samples include tissue samples, cell samples, bone marrow, and/or bodily fluids such as blood, saliva, semen, urine, and the like. Methods of obtaining samples and of isolating nucleic acids and proteins from sample are generally known to persons of skill in the art.

A “set” refers to a collection of one or more things. For example, a set may include 1, 2, 3, 4, 5, 10, 20, 50, 100, 1,000 or another number of genes or other types of molecules.

A “solid support” refers to a solid material that can be derivatized with, or otherwise attached to, a chemical moiety, such as an oligonucleotide probe or the like. Exemplary solid supports include plates (e.g., multi-well plates, etc.), beads, microbeads, tubes, fibers, whiskers, combs, hybridization chips (including microarray substrates, such as those used in GeneChip® probe arrays (Affymetrix, Inc., Santa Clara, Calif., USA) and the like), membranes, single crystals, ceramic layers, self-assembling monolayers, and the like.

“Specifically binding” means that a compound is capable of discriminating between two or more polynucleotides or polypeptides. For example, the compound binds to the desired polynucleotide or polypeptide, but essentially does not bind to a non-target polynucleotide or polypeptide. The compound can be an antibody, or a fragment thereof, an enzyme, a so-called small molecule compound, a protein-scaffold (e.g., an anticalin).

A “subject” refers to an organism. Typically, the organism is a mammalian organism, particularly a human organism.

The term “substantially identical” in the context of gene expression refers to levels of expression of genes that are approximately equal to one another. In some embodiments, for example, the expression levels of genes being compared are substantially identical to one another when they differ by less than about 5% (e.g., about 4%, about 3%, about 2%, about 1%, etc.).

“t(15;17)” refers to AML with translocation t(15;17) according to the WHO classification of haematological malignancies.

“t(8;21)” refers to AML with translocation t(8;21) according to the WHO classification of haematological malignancies.

“t(9;22)” refers to translocation (9;22).

The term “target” refers to an object that is the subject of analysis. In some embodiments, for example, targets are specific nucleic acid sequences (e.g., mRNAs of expressed genes, etc.), the presence, absence or abundance of which are to be determined. In certain embodiments, targets include polypeptides (e.g., proteins, etc.) of expressed genes. Typically, the sequences subjected to analysis are in or derived from “target cells”, such as a particular type of leukemia cell.

“Trisomy 8” refers to a condition in humans in which chromosome 8 is triploid in one or more cells.

### Introduction

The present invention provides methods, reagents, systems, and kits for classifying and prognosticating acute myeloid leukemia. In certain embodiments, for example, the methods include detecting an expression level of a set of genes in or derived from a target AML cell (e.g., an AML cell having an intermediate karyotype). These methods also include:
(a) correlating a detected differential expression of one or more genes selected from the markers listed in one or more of Tables 1-13 relative to a corresponding expression of the genes in or derived from at least one reference AML cell having a reciprocal translocation (e.g., a t(15;17), t(8;21), inv(16), t(11q23), inv(3), etc.) with the target AML cell having a CEBPA mutation;
(b) correlating a detected substantially identical expression of one or more genes selected from the markers listed in one or more of Tables 1-13 relative to a corresponding expression of the genes in or derived from at least one reference AML cell having a CEBPA mutation with the target AML cell having the CEBPA mutation;
(c) correlating a detected differential expression of one or more genes selected from the markers listed in one or more of Tables 1-13 relative to a corresponding expression of the genes in or derived from at least one reference AML cell having a CEBPA mutation with the target AML cell having a reciprocal translocation; or
(d) correlating a detected substantially identical expression of one or more genes selected from the markers listed in one or more of Tables 1-13 relative to a corresponding expression of the genes in or derived from at least one reference AML cell having a reciprocal translocation with the target AML cell having the reciprocal translocation, thereby classifying the AML cell.

In some embodiments, the set of genes is selected from one or more of: Table 1 (best 42 markers), Table 2 (top 100 markers to differentiate the favorable group from the unfavorable group), or Table 3 (top 100 differentially expressed markers between prognostic subgroups). The methods also include:
(a) correlating a detected a higher expression of an MPO gene and/or an ATBF1 gene in the target AML cell relative to a corresponding expression of the genes in or derived from an AML cell from a member of an unfavorable group with the subject having a probable overall survival rate at three years of about 55% or more; or,
(b) correlating a detected a higher expression of one or more of: an ETS2 gene, a RUNX1 gene, a TCF4 gene, a FOXC1 gene, a SFRS1 gene, a TPD52 gene, a NRIP1 gene, a TFPI gene, a UBL1 gene, an REC8L1 gene, an HSF2 gene, or an ETS2 gene in the target AML cell relative to a corresponding expression of the genes in or derived from an AML cell from a member of a favorable group with the subject having a probable overall survival rate at three years of about 25% or less.

The use of one or more of the markers described herein, e.g., utilizing a microarray technology or other gene expression profiling techniques, provides various advantages, including: (1) rapid and accurate diagnoses, (2) ease of use in laboratories without specialized knowledge, and (3) eliminates the need for analyzing viable cells for chromosome analysis, thereby eliminating cell sample transport issues. Aspects of the present invention are further illustrated in the examples provided below.

In practicing the present invention, many conventional techniques in, hematology, molecular biology and recombinant DNA are optionally used. These techniques are well known and are explained in, for example, Current Protocols in Molecular Biology, Volumes I, II, and III, 1997 (F. M. Ausubel ed.); Sambrook et al., Molecular Cloning: A Laboratory Manual, 3rd Ed., Cold Spring Harbor Laboratory Press, Cold Spring Harbor, N.Y., 2001; Berger and Kimmel, Guide to Molecular Cloning Techniques, Methods in Enzymology volume 152 Academic Press, Inc., San Diego, Calif. (Berger), DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); Greer et al. (Eds.), Wintrobe's Clinical Hematology, 11^{th} Ed., Lippincott Williams & Wilkins (2003); Shirlyn et al., Clinical Laboratory Hematology, Prentice Hall (2002); Lichtman et al., Williams Manual of Hematology, 6^{th} Ed., McGraw-Hill Professional (2002); and Methods in Enzymology Vol. 154 and Vol. 155 (Wu and Grossman, and Wu, eds., respectively), all of which are incorporated by reference.

In addition to the methods of the invention, the related kits and systems are also described further below.

### Sample Collection and Preparation

Samples are collected and prepared for analysis using essentially any technique known to those of skill in the art. In certain embodiments, for example, blood samples are obtained from subjects via venipuncture. Whole blood specimens are optionally collected in EDTA, Heparin or ACD vacutainer tubes. In other embodiments, the samples utilized for analysis comprise bone marrow aspirates, which are optionally processed, e.g., by erythrocyte lysis techniques, Ficoll density gradient centrifugations, or the like. Samples are typically either analyzed immediately following acquisition or stored frozen at, e.g., −80° C. until being subjected to analysis. Sample collection and handling are also described in, e.g., Garland et al., Handbook of Phlebotomy and Patient Service Techniques, Lippincott Williams & Wilkins (1998), and Slockbower et al. (Eds.), Collection and Handling of Laboratory Specimens: A Practical Guide, Lippincott Williams & Wilkins (1983), which are both incorporated by reference.

Treatment of Cells

The cells lines or sources containing the target nucleic acids and/or expression products thereof, are optionally subjected to one or more specific treatments that induce changes in gene expression, e.g., as part of processes to identify candidate modulators of gene expression. For example, a cell or cell line can be treated with or exposed to one or more chemical or biochemical constituents, e.g., pharmaceuticals, pollutants, DNA damaging agents, oxidative stress-inducing agents, pH-altering agents, membrane-disrupting agents, metabolic blocking agent, a chemical inhibitors, cell surface receptor ligands, antibodies, transcription promoters/enhancers/inhibitors, translation promoters/enhancers/inhibitors, protein-stabilizing or destabilizing agents, various toxins, carcinogens or teratogens, characterized or uncharacterized chemical libraries, proteins, lipids, or nucleic acids. Optionally, the treatment comprises an environmental stress, such as a change in one or more environmental parameters including, but not limited to, temperature (e.g. heat shock or cold shock), humidity, oxygen concentration (e.g., hypoxia), radiation exposure, culture medium composition, or growth saturation. Responses to these treatments may be followed temporally, and the treatment can be imposed for various times and at various concentrations. Target sequences can also be derived from cells exposed to multiple specific treatments as described above, either concurrently or in tandem (e.g., a cancerous cell or tissue sample may be further exposed to a DNA damaging agent while grown in an altered medium composition).

RNA Isolation

In some embodiments, total RNA is isolated from samples for use as target sequences. Cellular samples are lysed once culture with or without the treatment is complete by, for example, removing growth medium and adding a guanidinium-based lysis buffer containing several components to stabilize the RNA. In certain embodiments, the lysis buffer also contains purified RNAs as controls to monitor recovery and stability of RNA from cell cultures. Examples of such purified RNA templates include the Kanamycin Positive Control RNA from Promega (Madison, Wis., USA), and 7.5 kb Poly(A)-Tailed RNA from Life Technologies (Rockville, Md., USA). Lysates may be used immediately or stored frozen at, e.g., −80° C. Optionally, total RNA is purified from cell lysates (or other types of samples) using silica-based isolation in an automation-compatible, 96-well format, such as the Rneasy® purification platform (Qiagen, Inc. (Valencia, Calif., USA)). Alternatively, RNA is isolated using solid-phase oligo-dT capture using oligo-dT bound to microbeads or cellulose columns. This method has the added advantage of isolating mRNA from genomic DNA and total RNA, and allowing transfer of the mRNA-capture medium directly into the reverse transcriptase reaction. Other RNA isolation methods are contemplated, such as extraction with silica-coated beads or guanidinium. Further methods for RNA isolation and preparation can be devised by one skilled in the art.

Alternatively, the methods of the present invention are performed using crude cell lysates, eliminating the need to isolate RNA. RNAse inhibitors are optionally added to the crude samples. When using crude cellular lysates, genomic DNA could contribute one or more copies of target sequence, depending on the sample. In situations in which the target sequence is derived from one or more highly expressed genes, the signal arising from genomic DNA may not be significant. But for genes expressed at very low levels, the background can be eliminated by treating the samples with DNAse, or by using primers that target splice junctions. One skilled in the art can design a variety of specialized priming applications that would facilitate use of crude extracts as samples for the purposes of this invention.

### Gene Expression Profiling

The determination of gene expression levels may be effected at the transcriptional and/or translational level, i.e., at the level of mRNA or at the protein level. Essentially any method of gene expression profiling can be used or adapted for use in performing the methods described herein including, e.g., methods based on hybridization analysis of polynucleotides, and methods based on sequencing of polynucleotides. To illustrate, commonly used methods for the quantification of mRNA expression in a sample include northern blotting and in situ hybridization (Parker & Bames, Methods in Molecular Biology 106:247-283 (1999)), RNAse protection assays (Hod, Biotechniques 13:852-854 (1992)), and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). Optionally, molecular species, such as antibodies, aptamers, etc. that can specifically bind to proteins or fragments thereof are used for analysis (see, e.g., Beilharz et al., Brief Funct Genomic Proteomic 3(2):103-111 (2004)). Some of these techniques, with a certain degree of overlap in some cases, are described further below.

In certain embodiments, for example, the methods described herein include determining the expression levels of transcribed polynucleotides. In some of these embodiments, the transcribed polynucleotide is an mRNA, a cDNA and/or a cRNA. Transcribed polynucleotides are typically isolated from a sample, reverse transcribed and/or amplified, and labeled by techniques referred to above or otherwise known to persons skilled in the art. In order to determine the expression level of transcribed polynucleotides, the methods of the invention generally include hybridizing transcribed polynucleotides to a complementary polynucleotide, or a portion thereof, under a selected hybridization condition (e.g., a stringent hybridization condition), as described herein.

In some embodiments, the detection and quantification of amounts of polynucleotides to determine the level of expression of a marker are performed according to those described by, e.g., Sambrook et al., supra, or real time methods known in the art as 5′-nuclease methods disclosed in, e.g., WO 92/02638, U.S. Pat. No. 5,210,015, U.S. Pat. No. 5,804,375, and U.S. Pat. No. 5,487,972, which are each incorporated by reference. In some embodiments, for example, 5′-nuclease methods utilize the exonuclease activity of certain polymerases to generate signals. In these approaches, target nucleic acids are detected in processes that include contacting a sample with an oligonucleotide containing a sequence complementary to a region of the target nucleic acid component and a labeled oligonucleotide containing a sequence complementary to a second region of the same target nucleic acid component sequence strand, but not including the nucleic acid sequence defined by the first oligonucleotide, to create a mixture of duplexes during hybridization conditions, wherein the duplexes comprise the target nucleic acid annealed to the first oligonucleotide and to the labeled oligonucleotide such that the 3′-end of the first oligonucleotide is adjacent to the 5′-end of the labeled oligonucleotide. Then this mixture is treated with a template-dependent nucleic acid polymerase having a 5′ to 3′ nuclease activity under conditions sufficient to permit the to 3′ nuclease activity of the polymerase to cleave the annealed, labeled oligonucleotide and release labeled fragments. The signal generated by the hydrolysis of the labeled oligonucleotide is detected and/or measured. 5′-nuclease technology eliminates the need for a solid phase bound reaction complex to be formed and made detectable. Other exemplary methods include, e.g., fluorescence resonance energy transfer between two adjacently hybridized probes as used in the LightCycler® format described in, e.g., U.S. Pat. No. 6,174,670, which is incorporated by reference.

In one protocol, the marker, i.e., the polynucleotide, is in form of a transcribed nucleotide, where total RNA is isolated, cDNA and, subsequently, cRNA is synthesized and biotin is incorporated during the transcription reaction. The purified cRNA is applied to commercially available arrays that can be obtained from, e.g., Affymetrix, Inc. (Santa Clara, Calif. USA). The hybridized cRNA is optionally detected according to the methods described in the examples provided below. The arrays are produced by photolithography or other methods known to persons skilled in the art. Some of these techniques are also described in, e.g. U.S. Pat. No. 5,445,934, U.S. Pat. No. 5,744,305, U.S. Pat. No. 5,700,637, U.S. Pat. No. 5,945,334, EP 0 619 321, and EP 0 373 203, which are each incorporated by reference.

In another embodiment, the polynucleotide or at least one of the polynucleotides is in form of a polypeptide (e.g., expressed from the corresponding polynucleotide). The expression level of the polynucleotides or polypeptides is optionally detected using a compound that specifically binds to target polynucleotides or target polypeptides.

These and other exemplary gene expression profiling techniques are described further below.

Blotting Techniques

Some of the earliest expression profiling methods are based on the detection of a label in RNA hybrids or protection of RNA from enzymatic degradation (see, e.g., Ausubel et al., supra). Methods based on detecting hybrids include northern blots and slot/dot blots. These two techniques differ in that the components of the sample being analyzed are resolved by size in a northern blot prior to detection, which enables identification of more than one species simultaneously. Slot blots are generally carried out using unresolved mixtures or sequences, but can be easily performed in serial dilution, enabling a more quantitative analysis.

In Situ Hybridization

In situ hybridization is a technique that monitors transcription by directly visualizing RNA hybrids in the context of a whole cell. This method provides information regarding subcellular localization of transcripts (see, e.g., Suzuki et al., Pigment Cell Res. 17(1):10-4 (2004)).

Assays Based on Protection from Enzymatic Degradation

Techniques to monitor RNA that make use of protection from enzymatic degradation include S1 analysis and RNAse protection assays (RPAs). Both of these assays employ a labeled nucleic acid probe, which is hybridized to the RNA species being analyzed, followed by enzymatic degradation of single-stranded regions of the probe. Analysis of the amount and length of probe protected from degradation is used to determine the quantity and endpoints of the transcripts being analyzed.

Reverse Transcriptase PCR (RT-PCR) and Real-Time Detection

RT-PCR can be used to compare, e.g., mRNA levels in different sample populations, in normal and tumor tissues, with or without drug treatment, to characterize patterns of gene expression, to discriminate between closely related mRNAs, and to analyze RNA structure. These assays are derivatives of PCR in which amplification is preceded by reverse transcription of mRNA into cDNA. Accordingly, an initial step in these processes is generally the isolation of mRNA from a target sample (e.g., leukemia cells). The starting material is typically total RNA isolated from cancerous tissues or cells (e.g., bone marrow, peripheral blood aliquots, etc.), and in certain embodiments, from corresponding normal tissues or cells.

General methods for mRNA extraction are well known in the art and are disclosed in standard textbooks of molecular biology, including Ausubel et al., supra. Methods for RNA extraction from paraffin embedded tissues are disclosed, for example, in Rupp and Locker, Lab Invest. 56:A67 (1987), and De Andres et al., BioTechniques 18:42044 (1995), which are each incorporated by reference. In particular, RNA isolation can be performed using purification kit, buffer set and protease from commercial manufacturers, such as Qiagen, according to the manufacturer's instructions. For example, total RNA from cells in culture can be isolated using Qiagen Rneasy® mini-columns (referred to above). Other commercially available RNA isolation kits include MasterPure™ Complete DNA and RNA Purification Kit (EPICENTRE™, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

Since RNA generally cannot serve as a template for PCR, the process of gene expression profiling by RT-PCR typically includes the reverse transcription of the RNA template into cDNA, followed by its exponential amplification in a PCR reaction. Two commonly used reverse transcriptases are avilo myeloblastosis virus reverse transcriptase (AMV-RT) and Moloney murine leukemia virus reverse transcriptase (MMLV-RT). The reverse transcription-step is typically primed using specific primers, random hexamers, or oligo-dT primers, depending on the particular circumstances of expression profiling analysis. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Pairs of primers are generally used to generate amplicons in PCR reactions. A third oligonucleotide, or probe, is designed to bind to nucleotide sequence located between PCR primer pairs. Probe are generally non-extendible by Taq DNA polymerase enzyme, and are typically labeled with, e.g., a reporter fluorescent dye and a quencher fluorescent dye. Laser-induced emission from the reporter dye is quenched by the quenching dye when the two dyes are located close together, such as in an intact probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is typically liberated for each new molecule synthesized, and detection of the unquenched reporter dye provides the basis for quantitative interpretation of the data.

TaqMan® RT-PCR can be performed using commercially available equipment, such as, for example, a LightCycler® system (Roche Molecular Biochemicals, Mannheim, Germany) or an ABI PRISM 7700™ Sequence Detection System™ (Perkin-Elmer-Applied Biosystems, Foster City, Calif., USA).

To minimize errors and the effect of sample-to-sample variation, RT-PCR is typically performed using an internal standard. An ideal internal standard is expressed at a relatively constant level among different cells or tissues, and is unaffected by the experimental treatment. Exemplary RNAs frequently used to normalize patterns of gene expression are mRNAs transcribed from for the housekeeping genes glyceraldehyde-3-phosphate-dehydrogenase (GAPDH) and β-actin.

Other exemplary methods for targeted mRNA analysis include differential display reverse transcriptase PCR (DDRT-PCR) and RNA arbitrarily primed PCR (RAP-PCR) (see, e.g., U.S. Pat. No. 5,599,672; Liang and Pardee (1992) Science 257:967-971; Welsh et al. (1992) Nucleic Acids Res. 20:4965-4970, which are each incorporated by reference). Both methods use random priming to generate RT-PCR fingerprint profiles of transcripts in an unfractionated RNA preparation. The signal generated in these types of analyses is a pattern of bands separated on a sequencing gel. Differentially expressed genes appear as changes in the fingerprint profiles between two samples, which can be loaded in separate wells of the same gel. This type of readout allows identification of both up- and down-regulation of genes in the same reaction, appearing as either an increase or decrease in intensity of a band from one sample to another.

Molecular beacons are oligonucleotides designed for real time detection and quantification of target nucleic acids. The 5′ and 3′ termini of molecular beacons collectively comprise a pair of moieties, which confers the detectable properties of the molecular beacon. One of the termini is attached to a fluorophore and the other is attached to a quencher molecule capable of quenching a fluorescent emission of the fluorophore. To illustrate, one example fluorophore-quencher pair can use a fluorophore, such as EDANS or fluorescein, e.g., on the 5′-end and a quencher, such as Dabcyl, e.g., on the 3′-end. When the molecular beacon is present free in solution, i.e., not hybridized to a second nucleic acid, the stem of the molecular beacon is stabilized by complementary base pairing. This self-complementary pairing results in a “hairpin loop” structure for the molecular beacon in which the fluorophore and the quenching moieties are proximal to one another. In this confirmation, the fluorescent moiety is quenched by the quenching moiety. The loop of the molecular beacon typically comprises the oligonucleotide probe and is accordingly complementary to a sequence to be detected in the target microbial nucleic acid, such that hybridization of the loop to its complementary sequence in the target forces disassociation of the stem, thereby distancing the fluorophore and quencher from each other. This results in unquenching of the fluorophore, causing an increase in fluorescence of the molecular beacon.

Details regarding standard methods of making and using molecular beacons are well established in the literature and molecular beacons are available from a number of commercial reagent sources. Further details regarding methods of molecular beacon manufacture and use are found, e.g., in Leone et al. (1995) “Molecular beacon probes combined with amplification by NASBA enable homogenous real-time detection of RNA,” Nucleic Acids Res. 26:2150-2155; Kostrikis et al. (1998) “Molecular beacons: spectral genotyping of human alleles” Science 279:1228-1229; Fang et al. (1999) “Designing a novel molecular beacon for surface-immobilized DNA hybridization studies” J. Am. Chem. Soc. 121:2921-2922; and Marras et al. (1999) “Multiplex detection of single-nucleotide variation using molecular beacons” Genet. Anal. Biomol. Eng. 14:151-156, all of which are incorporated by reference. A variety of commercial suppliers produce standard and custom molecular beacons, including Oswel Research Products Ltd. (UK), Research Genetics (a division of Invitrogen, Huntsville, Ala., USA), the Midland Certified Reagent Company (Midland, Tex., USA), and Gorilla Genomics, LLC (Alameda, Calif., USA). A variety of kits which utilize molecular beacons are also commercially available, such as the Sentinel™ Molecular Beacon Allelic Discrimination Kits from Stratagene (La Jolla, Calif., USA) and various kits from Eurogentec SA (Belgium) and Isogen Bioscience BV (Netherlands).

Nucleic Acid Array-Based Analysis

Differential gene expression can also be identified, or confirmed using arrayed oligonucleotides (e.g., microarrays), which have the benefit of assaying for sample hybridization to a large number of probes in a highly parallel fashion. In these approaches, polynucleotide sequences of interest (e.g., probes, such as cDNAs, mRNAs, oligonucleotides, etc.) are plated, synthesized, or otherwise disposed on a microchip substrate or other type of solid support (see, e.g., U.S. Pat. Nos. 5,143,854 and 5,807,522; Fodor et al. (1991) Science 251:767-773; and Schena et al. (1995) Science 270:467-470, which are each incorporated by reference). Sequences of interest can be obtained, e.g., by creating a cDNA library from an mRNA source or by using publicly available databases, such as GenBank, to annotate the sequence information of custom cDNA libraries or to identify cDNA clones from previously prepared libraries. The arrayed sequences are then hybridized with target nucleic acids from cells or tissues of interest. As in the RT-PCR assays referred to above, the source of mRNA typically is total RNA isolated from a sample.

In certain embodiments, high-density oligonucleotide arrays are produced using a light-directed chemical synthesis process (i.e., photolithography). Unlike common cDNA arrays, oligonucleotide arrays (according, e.g., to the Affymetrix technology) typically use a single-dye technology. Given the sequence information of the probes or markers, the sequences are typically synthesized directly onto the array, thus, bypassing the need for physical intermediates, such as PCR products, commonly utilized in making cDNA arrays. For this purpose, selected markers, or partial sequences thereof, can be represented by, e.g., between about 14 to 20 features, typically by less then 14 features, more typically less then about 10 features, even more typically by about 6 features or less, with each feature generally being a short sequence of nucleotides (oligonucleotide), which is typically a perfect match (PM) to a segment of the respective gene. The PM oligonucleotides are paired with mismatch (MM) oligonucleotides, which have a single mismatch at the central base of the nucleotide and are used as “controls”. The chip exposure sites are typically defined by masks and are de-protected by the use of light, followed by a chemical coupling step resulting in the synthesis of one nucleotide. The masking, light deprotection, and coupling process can then be repeated to synthesize the next nucleotide, until the nucleotide chain is of the specified length.

To illustrate other embodiments of microarray-based assays, PCR amplified inserts of cDNA clones are applied to a substrate in a dense array. In some embodiments, for example, at least 10,000 different cDNA probe sequences are applied to a given solid support. Fluorescently labeled cDNA targets may be generated through incorporation of fluorescent nucleotides by reverse transcription of RNA extracted from the samples of interest. Labeled cDNA targets applied to the chip hybridize with corresponding probes on the array. After washing (e.g., under stringent conditions) to remove non-specifically bound probes, the chip is typically scanned by confocal laser microscopy or by another detection method, such as a CCD camera. Quantitation of hybridization of each arrayed element allows for assessment of corresponding mRNA abundance. With dual color fluorescence, for example, separately labeled cDNA probes generated from two sources of RNA can be hybridized concurrently to the arrayed probes. The relative abundance of the transcripts from the two sources corresponding to each specified gene can thus be determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996), which is incorporated by reference). Other microarray-based assay formats are also optionally utilized. Microarray analysis can be performed using commercially available equipment, following manufacturer's protocols, such as by using the Affymetrix GeneChip® technology, or Agilent's microarray technology.

If the polynucleotide being detected is mRNA, cDNA may be prepared into which a detectable label, as exemplified herein, is incorporated. For example, labeled cDNA, in single-stranded form, may then be hybridized (e.g., under stringent or highly stringent conditions) to a panel of single-stranded oligonucleotides representing different genes and affixed to a solid support, such as a chip. Upon applying appropriate washing steps, those cDNAs that have a counterpart in the oligonucleotide panel or array will be detected (e.g., quantitatively detected). Various advantageous embodiments of this general method are feasible. For example, mRNA or cDNA may be amplified, e.g., by a polymerase chain reaction or another nucleic acid amplification technique. In some embodiments, where quantitative assessments are sought, it is generally desirable that the number of amplified copies corresponds to the number of mRNAs originally present in the cell. Optionally, cDNAs are transcribed into cRNAs prior to hybridization steps in a given assay. In these embodiments, labels can be attached or incorporated cRNAs during or after the transcription step.

To further illustrate, one exemplary embodiment of the methods of the invention includes, as follows (1) obtaining a sample, e.g. bone marrow or peripheral blood aliquots, from a patient; (2) extracting RNA, e.g., mRNA, from the sample; (3) reverse transcribing the RNA into cDNA; (4) in vitro transcribing the cDNA into cRNA; (5) fragmenting the cRNA; (6) hybridizing the fragmented cRNA on selected microarrays (e.g., the HG-U133 microarray set available from Affymetrix, Inc. (Santa Clara, Calif. USA)); and (7) detecting hybridization.

Serical Analysis of Gene Expression (SAGE)

Serial analysis of gene expression (SAGE) is a method that allows the simultaneous and quantitative analysis of a large number of gene transcripts, without the need for providing an individual hybridization probe for each transcript. Initially, a short sequence tag (e.g., about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. SAGE-based assays are also described in, e.g. Velculescu et al., Science 270:484-487 (1995) and Velculescu et al., Cell 88:243-51 (1997), which are both incorporated by reference.

Gene Expression Analysis by Massively Parallel Signature Sequencing (MPSS)

These sequencing approaches generally combine non-gel-based signature sequencing with in vitro cloning of millions of templates on separate 5 μm diameter microbeads. Typically, a microbead library of DNA templates is constructed by in vitro cloning. This is generally followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method can be used to simultaneously and accurately provide, in a single operation, hundreds of thousands of gene signature sequences from cDNA libraries. MPSS is also described in, e.g., Brenner et al., (2000) Nature Biotechnology 18:630-634, which is incorporated by reference.

Immunoassays and Proteomics

Essentially any available technique for the detection of proteins is optionally utilized in the methods of the invention. Exemplary protein analysis technologies include, e.g., one- and two-dimensional SDS-PAGE-based separation and detection, immunoassays (e.g., western blotting, etc.), aptamer-based detection, mass spectrometric detection, and the like. These and other techniques are generally well-known in the art.

To illustrate, immunohistochemical methods are optionally used for detecting the expression levels of the targets described herein. Thus, antibodies or antisera (e.g., polyclonal antisera) and in certain embodiments, monoclonal antibodies specific for particular targets are used to detect expression. In some of these embodiments, antibodies are directly labeled, e.g., with radioactive labels, fluorescent labels, haptens, chemiluminescent dyes, enzyme substrates or co-factors, enzyme inhibitors, free radicals, enzymes (e.g., horseradish peroxidase or alkaline phosphatase), or the like. Such labeled reagents may be used in a variety of well known assays, such as radioimmunoassays, enzyme immunoassays, e.g., ELISA, fluorescent immunoassays, and the like. See, e.g., U.S. Pat. Nos. 3,766,162; 3,791,932; 3,817,837; and 4,233,402, which are each incorporated by reference. Additional labels are described further herein. Alternatively, unlabeled primary antibodies are used in conjunction with labeled secondary antibodies, comprising antisera, polyclonal antisera or a monoclonal antibody specific for the primary antibody. Immunohistochemistry protocols and kits are well known in the art and are commercially available.

To further illustrate, proteins from a cell or tissue under investigation may be contacted with a panel or array of aptamers or of antibodies or fragments or derivatives thereof. These biomolecules may be affixed to a solid support, such as a chip. The binding of proteins indicative of a given leukemia type or subtype is optionally verified by binding to a detectably labeled secondary antibody or aptamer. The labeling of antibodies is also described in, e.g., Harlow and Lane, Antibodies a laboratory manual, CSH Press (1988), which is incorporated by reference. To further illustrate, a minimum set of proteins necessary for detecting various leukemia types or subtypes may be selected for the creation of a protein array for use in making diagnoses with, e.g., protein lysates of bone marrow samples directly. Protein array systems for the detection of specific protein expression profiles are commercially available from various suppliers, including the Bio-Plex™ platform available from BIO-RAD Laboratories (Munich, Germany). In some embodiments of the invention, antibodies against the target proteins are produced and immobilized on a solid support, e.g., a glass slide or a well of a microtiter plate. The immobilized antibodies can be labeled with a reactant that is specific for the target proteins. These reactants can include, e.g., enzyme substrates, DNA, receptors, antigens or antibodies to create for example a capture sandwich immunoassay.

Target proteins can also be detected using aptamers including photoaptamers. Aptamers generally are single-stranded oligonucleotides (e.g., typically DNA for diagnostic applications) that assume a specific, sequence-dependent shape and binds to target proteins based on a “lock-and-key” fit between the two molecules. Aptamers can be identified using the SELEX process (Gold (1996) “The SELEX process: a surprising source of therapeutic and diagnostic compounds,” Harvey Lect. 91:47-57, which is incorporated by reference). Aptamer arrays are commercially available from various suppliers including, e.g., SomaLogic, Inc. (Boulder, Colo., USA).

The detection of proteins via mass includes various formats that can be adapted for use in the methods of the invention. Exemplary formats include matrix assisted laser desorption/ionization—(MALDI) and surface enhanced laser desorption/ionization-based (SELDI) detection. MALDI- and SELDI-based detection are also described in, e.g., Weinberger et al. (2000) “Recent trends in protein biochip technology,” Pharmacogenomics 1(4):395-416, Forde et al. (2002) “Characterization of transcription factors by mass spectrometry and the role of SELDI-MS,” Mass Spectrom. Rev. 21(6):419-439, and Leushner (2001) “MALDI TOF mass spectrometry: an emerging platform for genomics and diagnostics,” Expert Rev. Mol. Diagn. 1(1): 11-18, which are each incorporated by reference. Protein chips and related instrumentation are available from commercial suppliers, such as Ciphergen Biosystems, Inc. (Fremont, Calif., USA).

### Oligonucleotide Preparation

Various approaches can be utilized by one of skill in the art to design oligonucleotides for use as probes and/or primers. To illustrate, the DNAstar software package available from DNASTAR, Inc. (Madison, Wis.) can be used for sequence alignments. For example, target nucleic acid sequences and non-target nucleic acid sequences can be uploaded into DNAstar EditSeq program as individual files, e.g., as part of a process to identify regions in these sequences that have low sequence similarity. To further illustrate, pairs of sequence files can be opened in the DNAstar MegAlign sequence alignment program and the Clustal W method of alignment can be applied. The parameters used for Clustal W alignments are optionally the default settings in the software. MegAlign typically does not provide a summary of the percent identity between two sequences. This is generally calculated manually. From the alignments, regions having, e.g., less than 85% identity with one another are typically identified and oligonucleotide sequences in these regions can be selected. Many other sequence alignment algorithms and software packages are also optionally utilized. Sequence alignment algorithms are also described in, e.g., Mount, Bioinformatics: Sequence and Genome Analysis, Cold Spring Harbor Laboratory Press (2001), and Durbin et al., Biological Sequence Analysis: Probabilistic Models of Proteins and Nucleic Acids, Cambridge University Press (1998), which are both incorporated by reference.

To further illustrate, optimal alignment of sequences for comparison can be conducted, e.g., by the local homology algorithm of Smith & Waterman (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, which are each incorporated by reference, and by computerized implementations of these algorithms (e.g., GAP, BESTFIT, FASTA, and TFASTA in the Wisconsin Genetics Software Package, Genetics Computer Group (Madison, Wis.)), or by even by visual inspection.

Another example algorithm that is suitable for determining percent sequence identity is the BLAST algorithm, which is described in, e.g., Altschul et al. (1990) J. Mol. Biol. 215:403-410, which is incorporated by reference. Software for performing versions of BLAST analyses is publicly available through the National Center for Biotechnology Information on the world wide web at ncbi.nlm.nih.gov/ as of Nov. 4, 2004.

An additional example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-360, which is incorporated by reference.

Oligonucleotide probes and primers are optionally prepared using essentially any technique known in the art. In certain embodiments, for example, the oligonucleotide probes and primers are synthesized chemically using essentially any nucleic acid synthesis method, including, e.g., according to the solid phase phosphoramidite method described by Beaucage and Caruthers (1981) Tetrahedron Letts. 22(20): 1859-1862, which is incorporated by reference. To further illustrate, oligonucleotides can also be synthesized using a triester method (see, e.g., Capaldi et al. (2000) “Highly efficient solid phase synthesis of oligonucleotide analogs containing phosphorodithioate linkages” Nucleic Acids Res. 28(9):e40 and Eldrup et al. (1994) “Preparation of oligodeoxyribonucleoside phosphorodithioates by a triester method” Nucleic Acids Res. 22(10):1797-1804, which are both incorporated by reference). Other synthesis techniques known in the art can also be utilized, including, e.g., using an automated synthesizer, as described in Needham-VanDevanter et al. (1984) Nucleic Acids Res. 12:6159-6168, which is incorporated by reference. A wide variety of equipment is commercially available for automated oligonucleotide synthesis. Multi-nucleotide synthesis approaches (e.g., tri-nucleotide synthesis, etc.) are also optionally utilized. Moreover, the primer nucleic acids optionally include various modifications. In certain embodiments, for example, primers include restriction site linkers, e.g., to facilitate subsequent amplicon cloning or the like. To further illustrate, primers are also optionally modified to improve the specificity of amplification reactions as described in, e.g., U.S. Pat. No. 6,001,611, entitled “MODIFIED NUCLEIC ACID AMPLIFICATION PRIMERS,” issued Dec. 14, 1999 to Will, which is incorporated by reference. Primers and probes can also be synthesized with various other modifications as described herein or as otherwise known in the art.

Probes and/or primers utilized in the methods and other aspects of the invention are typically labeled to permit detection of probe-target hybridization duplexes. In general, a label can be any moiety that can be attached to a nucleic acid and provide a detectable signal (e.g., a quantifiable signal). Labels may be attached to oligonucleotides directly or indirectly by a variety of techniques known in the art. To illustrate, depending on the type of label used, the label can be attached to a terminal (5′ or 3′ end of an oligonucleotide primer and/or probe) or a non-terminal nucleotide, and can be attached indirectly through linkers or spacer arms of various sizes and compositions. Using commercially available phosphoramidite reagents, one can produce oligonucleotides containing functional groups (e.g., thiols or primary amines) at either the 5′ or 3′ terminus via an appropriately protected phosphoramidite, and can label such oligonucleotides using protocols described in, e.g., Innis et al. (Eds.) PCR Protocols: A Guide to Methods and Applications, Elsevier Science & Technology Books (1990) (Innis), which is incorporated by reference.

Essentially any labeling moiety is optionally utilized to label a probe and/or primer by techniques well known in the art. In some embodiments, for example, labels comprise a fluorescent dye (e.g., a rhodamine dye (e.g., R6G, R110, TAMRA, ROX, etc.), a fluorescein dye (e.g., JOE, VIC, TET, HEX, FAM, etc.), a halofluorescein dye, a cyanine dye (e.g., CY3, CY3.5, CY5, CY5.5, etc.), a BODIPY® dye (e.g., FL, 530/550, TR, TMR, etc.), an ALEXA FLUOR® dye (e.g., 488, 532, 546, 568, 594, 555, 653, 647, 660, 680, etc.), a dichlororhodamine dye, an energy transfer dye (e.g., BIGDYE™ v 1 dyes, BIGDYE™ v 2 dyes, BIGDYE™ v 3 dyes, etc.), Lucifer dys yellow, etc.), CASCADE BLUE®, Oregon Green, and the like. Additional examples of fluorescent dyes are provided in, e.g., Haugland, Molecular Probes Handbook of Fluorescent Probes and Research Products, Ninth Ed. (2003) and the updates thereto, which are each incorporated by reference. Fluorescent dyes are generally readily available from various commercial suppliers including, e.g., Molecular Probes, Inc. (Eugene, Oreg.), Amersham Biosciences Corp. (Piscataway, N.J.), Applied Biosystems (Foster City, Calif.), etc. Other labels include, e.g., biotin, weakly fluorescent labels (Yin et al. (2003) Appl Environ Microbiol. 69(7):3938, Babendure et al. (2003) Anal. Biochem. 317(1):1, and Jankowiak et al. (2003) Chem Res Toxicol. 16(3):304), non-fluorescent labels, colorimetric labels, chemiluminescent labels (Wilson et al. (2003) Analyst. 128(5):480 and Roda et al. (2003) Luminescence 18(2):72), Raman labels, electrochemical labels, bioluminescent labels (Kitayama et al. (2003). Photochem Photobiol. 77(3):333, Arakawa et al. (2003) Anal. Biochem. 314(2):206, and Maeda (2003) J. Pharm. Biomed. Anal. 30(6):1725), and an alpha-methyl-PEG labeling reagent as described in, e.g., U.S. Provisional Patent Application No. 60/428,484, filed on Nov. 22, 2002, which references are each incorporated by reference. Nucleic acid labeling is also described further below. In some embodiments, labeling is achieved using synthetic nucleotides (e.g., synthetic ribonucleotides, etc.) and/or recombinant phycoerythrin (PE).

In addition, whether a fluorescent dye is a label or a quencher is generally defined by its excitation and emission spectra, and the fluorescent dye with which it is paired. Fluorescent molecules commonly used as quencher moieties in probes and primers include, e.g., fluorescein, FAM, JOE, rhodamine, R6G, TAMRA, ROX, DABCYL, and EDANS. Many of these compounds are available from the commercial suppliers referred to above. Exemplary non-fluorescent or dark quenchers that dissipate energy absorbed from a fluorescent dye include the Black Hole Quenchers™ or BHQ™, which are commercially available from Biosearch Technologies, Inc. (Novato, Calif., USA).

To further illustrate, essentially any nucleic acid (and virtually any labeled nucleic acid, whether standard or non-standard) can be custom or standard ordered from any of a variety of commercial sources, such as The Midland Certified Reagent Company, The Great American Gene Company, ExpressGen Inc., Operon Technologies Inc., Proligo LLC, and many others.

In certain embodiments, modified nucleotides are included in probes and primers. To illustrate, the introduction of modified nucleotide substitutions into oligonucleotide sequences can, e.g., increase the melting temperature of the oligonucleotides. In some embodiments, this can yield greater sensitivity relative to corresponding unmodified oligonucleotides even in the presence of one or more mismatches in sequence between the target nucleic acid and the particular oligonucleotide. Exemplary modified nucleotides that can be substituted or added in oligonucleotides include, e.g., C5-ethyl-dC, C5-methyl-dU, C5-ethyl-dU, 2,6-diaminopurines, C5-propynyl-dC, C7-propynyl-dA, C7-propynyl-dG, C5-propargylamino-dC, C5-propargylamino-dU, C7-propargylamino-dA, C7-propargylamino-dG, 7-deaza-2-deoxyxanthosine, pyrazolopyrimidine analogs, pseudo-dU, nitro pyrrole, nitro indole, 2′-0-methyl Ribo-U, 2′-0-methyl Ribo-C, an 8-aza-dA, an 8-aza-dG, a 7-deaza-dA, a 7-deaza-dG, N4-ethyl-dC, N6-methyl-dA, etc. To further illustrate, other examples of modified oligonucleotides include those having one or more LNA™ monomers. Nucleotide analogs such as these are also described in, e.g., U.S. Pat. No. 6,639,059, entitled “SYNTHESIS OF [2.2.1]BICYCLO NUCLEOSIDES,” issued Oct. 28, 2003 to Kochkine et al., U.S. Pat. No. 6,303,315, entitled “ONE STEP SAMPLE PREPARATION AND DETECTION OF NUCLEIC ACIDS IN COMPLEX BIOLOGICAL SAMPLES,” issued Oct. 16, 2001 to Skouv, and U.S. Pat. Application Pub. No. 2003/0092905, entitled “SYNTHESIS OF [2.2.1]BICYCLO NUCLEOSIDES,” by Kochkine et al. that published May 15, 2003, which are each incorporated by reference. Oligonucleotides comprising LNA™ monomers are commercially available through, e.g., Exiqon A/S (Vedbaek, DK). Additional oligonucleotide modifications are referred to herein, including in the definitions provided above.

### Array Formats

In certain embodiments, oligonucleotide probes designed to hybridize with target nucleic acids are covalently or noncovalently attached to solid supports. In these embodiments, labeled amplicons derived from patient samples are typically contacted with these solid support-bound probes to effect hybridization and detection. In other embodiments, amplicons are attached to solid supports and contacted with labeled probes. Optionally, antibodies, aptamers, or other probe biomolecules utilized in a given assay are similarly attached to solid supports.

Essentially any substrate material can be adapted for use as a solid support. In certain embodiments, for example, substrates are fabricated from silicon, glass, or polymeric materials (e.g., glass or polymeric microscope slides, silicon wafers, wells of microwell plates, etc.). Suitable glass or polymeric substrates, including microscope slides, are available from various commercial suppliers, such as Fisher Scientific (Pittsburgh, Pa., USA) or the like. In some embodiments, solid supports utilized in the invention are membranes. Suitable membrane materials are optionally selected from, e.g. polyaramide membranes, polycarbonate membranes, porous plastic matrix membranes (e.g., POREX® Porous Plastic, etc.), nylon membranes, ceramic membranes, polyester membranes, polytetrafluoroethylene (TEFLON®) membranes, nitrocellulose membranes, or the like. Many of these membranous materials are widely available from various commercial suppliers, such as, P. J. Cobert Associates, Inc. (St. Louis, Mo., USA), Millipore Corporation (Bedford, Mass., USA), or the like. Other exemplary solid supports that are optionally utilized include, e.g., ceramics, metals, resins, gels, plates, beads (e.g., magnetic microbeads, etc.), whiskers, fibers, combs, single crystals, self-assembling monolayers, and the like.

Nucleic acids are directly or indirectly (e.g., via linkers, such as bovine serum albumin (BSA) or the like) attached to the supports, e.g., by any available chemical or physical method. A wide variety of linking chemistries are available for linking molecules to a wide variety of solid supports. More specifically, nucleic acids may be attached to the solid support by covalent binding, such as by conjugation with a coupling agent or by non-covalent binding, such as electrostatic interactions, hydrogen bonds or antibody-antigen coupling, or by combinations thereof. Typical coupling agents include biotin/avidin, biotin/streptavidin, Staphylococcus aureus protein A/IgG antibody F_{c} fragment, and streptavidin/protein A chimeras (Sano et al. (1991) Bio/Technology 9:1378, which is incorporated by reference), or derivatives or combinations of these agents. Nucleic acids may be attached to the solid support by a photocleavable bond, an electrostatic bond, a disulfide bond, a peptide bond, a diester bond or a combination of these bonds. Nucleic acids are also optionally attached to solid supports by a selectively releasable bond such as 4,4′-dimethoxytrityl or its derivative.

Cleavable attachments can be created by attaching cleavable chemical moieties between the probes and the solid support including, e.g., an oligopeptide, oligonucleotide, oligopolyamide, oligoacrylamide, oligoethylene glycerol, alkyl chains of between about 6 to 20 carbon atoms, and combinations thereof. These moieties may be cleaved with, e.g., added chemical agents, electromagnetic radiation, or enzymes. Exemplary attachments cleavable by enzymes include peptide bonds, which can be cleaved by proteases, and phosphodiester bonds which can be cleaved by nucleases.

Chemical agents such as β-mercaptoethanol, dithiothreitol (DTT) and other reducing agents cleave disulfide bonds. Other agents which may be useful include oxidizing agents, hydrating agents and other selectively active compounds. Electromagnetic radiation such as ultraviolet, infrared and visible light cleave photocleavable bonds. Attachments may also be reversible, e.g., using heat or enzymatic treatment, or reversible chemical or magnetic attachments. Release and reattachment can be performed using, e.g., magnetic or electrical fields.

A number of array systems have been described and can be adapted for use in the detection of target microbial nucleic acids. Aspects of array construction and use are also described in, e.g., Sapolsky et al. (1999) “High-throughput polymorphism screening and genotyping with high-density oligonucleotide arrays” Genetic Analysis: Biomolecular Engineering 14:187-192, Lockhart (1998) “Mutant yeast on drugs” Nature Medicine 4:1235-1236, Fodor (1997) “Genes, Chips and the Human Genome” FASEB Journal 11:A879, Fodor (1997) “Massively Parallel Genomics” Science 277: 393-395, and Chee et al. (1996) “Accessing Genetic Information with High-Density DNA Arrays” Science 274:610-614, all of which are incorporated by reference.

### Nucleic Acid Hybridization

The length of complementary region or sequence between primer or probes and their binding partners (e.g., target nucleic acids) should generally be sufficient to allow selective or specific hybridization of the primers or probes to the targets at the selected annealing temperatures used for a particular nucleic acid amplification protocol, expression profiling assay, etc. Although other lengths are optionally utilized, complementary regions of, for example, between about 10 and about 50 nucleotides (e.g., about 15, 16, 17, 18, 19, 20, 21, 22, 23, 24, or 25 or more nucleotides) are typically used in a given application.

“Stringent hybridization wash conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

For purposes of the present invention, generally, “highly stringent” hybridization and wash conditions are selected to be about 5° C. or less lower than the thermal melting point (Tₘ) for the specific sequence at a defined ionic strength and pH (as noted below, highly stringent conditions can also be referred to in comparative terms). The Tₘ is the temperature (under defined ionic strength and pH) at which 50% of the test sequence hybridizes to a perfectly matched primer or probe. Very stringent conditions are selected to be equal to the Tₘ for a particular primer or probe.

The Tₘ is the temperature of the nucleic acid duplexes indicates the temperature at which the duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides.

After hybridization, unhybridized nucleic acid material can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lowers the background signal, typically with only the specific signal remaining. See, e.g., Rapley et al. (Eds.), Molecular Biomethods Handbook (Humana Press, Inc. 1998), which is incorporated by reference.

Thus, one measure of stringent hybridization is the ability of the primer or probe to hybridize to one or more of the target nucleic acids (or complementary polynucleotide sequences thereof) under highly stringent conditions. Stringent hybridization and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria is met. For example, the hybridization and wash conditions are gradually increased until a target nucleic acid, and complementary polynucleotide sequences thereof, binds to a perfectly matched complementary nucleic acid.

A target nucleic acid is said to specifically hybridize to a primer or probe nucleic acid when it hybridizes at least as well to the primer or probe as to a perfectly matched complementary target, i.e., with a signal to noise ratio at least 1/2 as high as hybridization of the primer or probe to the target under conditions in which the perfectly matched primer or probe binds to the perfectly matched complementary target with a signal to noise ratio that is at least about 2.5×-10×, typically 5×-10× as high as that observed for hybridization to any of the unmatched target nucleic acids.

### Nucleic Acid Amplification

In some embodiments, RNA is converted to cDNA in a reverse-transcription (RT) reaction using, e.g., a target-specific primer complementary to the RNA for each gene target being monitored. Methods of reverse transcribing RNA into cDNA are well known, and described in Sambrook, supra. Alternative methods for reverse transcription utilize thermostable DNA polymerases, as described in the art. As an exemplary embodiment, avian myeloblastosis virus reverse transcriptase (AMV-RT), or Maloney murine leukemia virus reverse transcriptase (MoMLV-RT) is used, although other enzymes are also optionally utilized. An advantage of using target-specific primers in the RT reaction is that only the desired sequences are converted into a PCR template. Superfluous primers or cDNA products are generally not carried into subsequent PCR amplifications.

In another embodiment, RNA targets are reverse transcribed using non-specific primers, such as an anchored oligo-dT primer, or random sequence primers. An advantage of this embodiment is that the “unfractionated” quality of the mRNA sample is maintained because the sites of priming are non-specific, i.e., the products of this RT reaction will serve as template for any desired target in the subsequent PCR amplification. This allows samples to be archived in the form of DNA, which is more stable than RNA.

In other embodiments, transcription-based amplification systems (TAS) are used, such as that first described by Kwoh et al. (Proc. Natl. Acad. Sci. (1989) 86(4): 1173-7), or isothermal transcription-based systems such as 3SR (Self-Sustained Sequence Replication; Guatelli et al. (1990) Proc. Natl. Acad. Sci. 87:1874-1878) or NASBA (nucleic acid sequence based amplification; Kievits et al. (1991) J Virol Methods. 35(3):273-86), which are each incorporated by reference. In these methods, the mRNA target of interest is copied into cDNA by a reverse transcriptase. The primer for cDNA synthesis includes the promoter sequence of a designated DNA-dependent RNA polymerase 5′ to the primer's region of homology with the template. The resulting cDNA products can then serve as templates for multiple rounds of transcription by the appropriate RNA polymerase. Transcription of the cDNA template rapidly amplifies the signal from the original target mRNA. The isothermal reactions bypass the need for denaturing cDNA strands from their RNA templates by including RNAse H to degrade RNA hybridized to DNA.

In other exemplary embodiments, amplification is accomplished by used of the ligase chain reaction (LCR), disclosed in European Patent Application No. 320,308 (Backman and Wang), or by the ligase detection reaction (LDR), disclosed in U.S. Pat. No. 4,883,750 (Whiteley et al.), which are each incorporated by reference. In LCR, two probe pairs are typically prepared, which are complimentary each other, and to adjacent sequences on both strands of the target. Each pair will bind to opposite strands of the target such that they abut. Each of the two probe pairs can then be linked to form a single unit, using a thermostable ligase. By temperature cycling, as in PCR, bound ligated units dissociate from the target, then both molecules can serve as “target sequences” for ligation of excess probe pairs, providing for an exponential amplification. The LDR is very similar to LCR. In this variation, oligonucleotides complimentary to only one strand of the target are used, resulting in a linear-amplification of ligation-products, since only the original target DNA can serve as a hybridization template. It is used following a PCR amplification of the target in order to increase signal.

In further embodiments, several methods generally known in the art would be suitable methods of amplification. Some additional examples include, but are not limited to, strand displacement amplification (Walker et al. (1992) Nucleic Acids Res. 20:1691-1696), repair chain reaction (REF), cyclic probe reaction (REF), solid-phase amplification, including bridge amplification (Mehta and Singh (1999) BioTechniques 26(6): 1082-1086), rolling circle amplification (Kool, U.S. Pat. No. 5,714,320), rapid amplification of cDNA ends (Frohman (1988) Proc. Natl. Acad. Sci. 85: 8998-9002), and the “invader assay” (Griffin et al. (1999) Proc. Natl. Acad. Sci. 96: 6301-6306), which are each incorporated by reference. Amplicons are optionally recovered and purified from other reaction components by any of a number of methods well known in the art, including electrophoresis, chromatography, precipitation, dialysis, filtration, and/or centrifugation. Aspects of nucleic acid purification are described in, e.g., Douglas et al., DNA Chromatography, Wiley, John & Sons, Inc. (2002), and Schott, Affinity Chromatography: Template Chromatography of Nucleic Acids and Proteins, Chromatographic Science Series, #27, Marcel Dekker (1984), both of which are incorporated by reference. In certain embodiments, amplicons are not purified prior to detection, such as when amplicons are detected simultaneous with amplification.

### Data Collection

The number of species than can be detected within a mixture depends primarily on the resolution capabilities of the separation platform used, and the detection methodology employed. In some embodiments, separation steps are is based upon size-based separation technologies. Once separated, individual species are detected and quantitated by either inherent physical characteristics of the molecules themselves, or detection of an associated label.

Embodiments employing other separation methods are also described. For example, certain types of labels allow resolution of two species of the same mass through deconvolution of the data. Non-size based differentiation methods (such as deconvolution of data-from-overlapping signals generated by two different fluorophores) allow pooling of a plurality of multiplexed reactions to further increase throughput.

Separation Methods

Certain embodiments of the invention incorporate a step of separating the products of a reaction based on their size differences. The PCR products generated during an amplification reaction typically range from about 50 to about 500 bases in length, which can be resolved from one another by size. Any one of several devices may be used for size separation, including mass spectrometry, any of several electrophoretic devices, including capillary, polyacrylamide gel, or agarose gel electrophoresis, or any of several chromatographic devices, including column chromatography, HPLC, or FPLC.

In some embodiments, sample analysis includes the use of mass spectrometry. Several modes of separation that determine mass are possible, including Time-of-Flight (TOF), Fourier Transform Mass Spectrometry (FTMS), and quadruple mass spectrometry. Possible methods of ionization include Matrix-Assisted Laser Desorption and Ionization (MALDI) or Electrospray Ionization (ESI). A preferred embodiment for the uses described in this invention is MALDI-TOF (Wu, et al. (1993) Rapid Communications in Mass Spectrometry 7:142-146, which is incorporated by reference). This method may be used to provide unfragmented mass spectra of mixed-base oligonucleotides containing between about 1 and about 1000 bases. In preparing the sample for analysis, the analyte is mixed into a matrix of molecules that resonantly absorb light at a specified wavelength. Pulsed laser light is then used to desorb oligonucleotide molecules out of the absorbing solid matrix, creating free, charged oligomers and minimizing fragmentation. An exemplary solid matrix material for this purpose is 3-hydroxypicolinic acid (Wu, supra), although others are also optionally used.

In another embodiment, a microcapillary is used for analysis of nucleic acids obtained from the sample. Microcapillary electrophoresis generally involves the use of a thin capillary or channel, which may optionally be filled with a particular medium to improve separation, and employs an electric field to separate components of the mixture as the sample travels through the capillary. Samples composed of linear polymers of a fixed charge-to-mass ratio, such as DNA or RNA, will separate based on size. The high surface to volume ratio of these capillaries allows application of very high electric fields across the capillary without substantial thermal variation, consequently allowing very rapid separations. When combined with confocal imaging methods, these methods provide sensitivity in the range of attomoles, comparable to the sensitivity of radioactive sequencing methods. The use of microcapillary electrophoresis in size separation of nucleic acids has been reported in Woolley and Mathies (Proc. Natl. Acad. Sci. USA (1994) 91:11348-11352), which is incorporated by reference. Capillaries are optionally fabricated from fused silica, or etched, machined, or molded into planar substrates. In many microcapillary electrophoresis methods, the capillaries are filled with an appropriate separation/sieving matrix. Several sieving matrices are known in the art that may be used for this application, including, e.g., hydroxyethyl cellulose, polyacrylamide, agarose, and the like. Generally, the specific gel matrix, running buffers and running conditions are selected to obtain the separation required for a particular application. Factors that are considered include, e.g., sizes of the nucleic acid fragments, level of resolution, or the presence of undenatured nucleic acid molecules. For example, running buffers may include agents such as urea to denature double-stranded nucleic acids in a sample.

Microfluidic systems for separating molecules such as DNA and RNA are commercially available and are optionally employed in the methods of the present invention. For example, the “Personal Laboratory System” and the “High Throughput System” have been developed by Caliper Lifesciences Corp. (Mountain View, Calif.). The Agilent 2100, which uses Caliper Lifesciences' LabChip™ microfluidic systems, is available from Agilent Technologies (Palo Alto, Calif., USA). Currently, specialized microfluidic devices, which provide for rapid separation and analysis of both DNA and RNA are available from Caliper Lifesciences for the Agilent 2100.

Other embodiments are generally known in the art for separating PCR amplification products by electrophoresis through gel matrices. Examples include polyacrylamide, agarose-acrylamide, or agarose gel electrophoresis, using standard methods (Sambrook, supra).

Alternatively, chromatographic techniques may be employed for resolving amplification products. Many types of physical or chemical characteristics may be used to effect chromatographic separation in the present invention, including adsorption, partitioning (such as reverse phase), ion-exchange, and size exclusion. Many specialized techniques have been developed for their application including methods utilizing liquid chromatography or HPLC (Katz and Dong (1990) BioTechniques 8(5):546-55; Gaus et al. (1993) J. Immunol. Methods 158:229-236). In yet another embodiment, cDNA products are captured by their affinity for certain substrates, or other incorporated binding properties. For example, labeled cDNA products such as biotin or antigen can be captured with beads bearing avidin or antibody, respectively. Affinity capture is utilized on a solid support to enable physical separation. Many types of solid supports are known in the art that would be applicable to the present invention. Examples include beads (e.g. solid, porous, magnetic), surfaces (e.g. plates, dishes, wells, flasks, dipsticks, membranes), or chromatographic materials (e.g. fibers, gels, screens).

Certain separation embodiments entail the use of microfluidic techniques. Technologies include separation on a microcapillary platform, such as designed by ACLARA BioSciences Inc. (Mountain View, Calif.), or the LabChip™ microfluidic devices made by Caliper Lifesciences Corp. Another technology developed by Nanogen, Inc. (San Diego, Calif.), utilizes microelectronics to move and concentrate biological molecules on a semiconductor microchip. The microfluidics platforms developed at Orchid Biosciences, Inc. (Princeton, N.J.), including the Chemtel™ Chip, which provides for parallel processing of hundreds of reactions, can also be used in certain embodiments. These microfluidic platforms require only nanoliter sample volumes, in contrast to the microliter volumes required by other conventional separation technologies.

Some of the processes usually involved in genetic analysis have been miniaturized using microfluidic devices. For example, PCT publication WO 94/05414 reports an integrated micro-PCR apparatus for collection and amplification of nucleic acids from a specimen. U.S. Pat. No. 5,304,487 (Wilding et al.) and U.S. Pat. No. 5,296,375 (Kricka et al.) discuss devices for collection and analysis of cell-containing samples. U.S. Pat. No. 5,856,174 (Lipshutz et al.) describes an apparatus that combines the various processing and analytical operations involved in nucleic acid analysis. Each of these references is incorporated by reference.

Additional technologies are also contemplated. For example, Kasianowicz et al. (Proc. Natl. Acad. Sci. USA (1996) 93:13770-13773, which is incorporated by reference) describes the use of ion channel pores in a lipid bilayer membrane for determining the length of polynucleotides. In this system, an electric field is generated by the passage of ions through the pores. Polynucleotide lengths are measured as a transient decrease of ionic current due to blockage of ions passing through the pores by the nucleic acid. The duration of the current decrease was shown to be proportional to polymer length. Such a system can be applied as a size separation platform in certain embodiments of the present invention.

Primers are useful both as reagents for hybridization in solution, such as priming PCR amplification, as well as for embodiments employing a solid phase, such as microarrays. With microarrays, sample nucleic acids such as mRNA or DNA are fixed on a selected matrix or surface. PCR products may be attached to the solid surface via one of the amplification primers, then denatured to provide single-stranded DNA. This spatially-partitioned, single-stranded nucleic acid is then subject to hybridization with selected probes under conditions that allow a quantitative determination of target abundance. In this embodiment, amplification products from each individual reaction are not physically separated, but are differentiated by hybridizing with a set of probes that are differentially labeled. Alternatively, unextended amplification primers may be physically immobilized at discreet positions on the solid support, then hybridized with the products of a nucleic acid amplification for quantitation of distinct species within the sample. In this embodiment, amplification products are separated by way of hybridization with probes that are spatially separated on the solid support.

Separation platforms may optionally be coupled to utilize two different separation methodologies, thereby increasing the multiplexing capacity of reactions beyond that which can be obtained by separation in a single dimension. For example, some of the RT-PCR primers of a multiplex reaction may be coupled with a moiety that allows affinity capture, while other primers remain unmodified. Samples are then passed through an affinity chromatography column to separate PCR products arising from these two classes of primers. Flow-through fractions are collected and the bound fraction eluted. Each fraction may then be further separated based on other criteria, such as size, to identify individual components.

Detection Methods

Following separation of the different products of a multiplex amplification, one or more of the amplicons are detected and/or quantitated. Some embodiments of the methods of the present invention enable direct detection of products. Other embodiments detect reaction products via a label associated with one or more of the amplification primers. Many types of labels suitable for use in the present invention are known in the art, including chemiluminescent, isotopic, fluorescent, electrochemical, inferred, or mass labels, or enzyme tags. In further embodiments, separation and detection may be a multi-step process in which samples are fractionated according to more than one property of the products, and detected one or more stages during the separation process.

An exemplary embodiment of the invention that does not use labeling or modification of the molecules being analyzed is detection of the mass-to-charge ratio of the molecule itself. This detection technique is optionally used when the separation platform is a mass spectrometer. An embodiment for increasing resolution and throughput with mass detection is in mass-modifying the amplification products. Nucleic acids can be mass-modified through either the amplification primer or the chain-elongating nucleoside triphosphates. Alternatively, the product mass can be shifted without modification of the individual nucleic acid components, by instead varying the number of bases in the primers. Several types of moieties have been shown to be compatible with analysis by mass spectrometry, including polyethylene glycol, halogens, alkyl, aryl, or aralkyl moieties, peptides (described in, for example, U.S. Pat. No. 5,691,141, which is incorporated by reference). Isotopic variants of specified atoms, such as radioisotopes or stable, higher mass isotopes, are also used to vary the mass of the amplification product. Radioisotopes can be detected based on the energy released when they decay, and numerous applications of their use are generally known in the art. Stable (non-decaying) heavy isotopes can be detected based on the resulting shift in mass, and are useful for distinguishing between two amplification products that would otherwise have similar or equal masses. Other embodiments of detection that make use of inherent properties of the molecule being analyzed include ultraviolet light absorption (UV) or electrochemical detection. Electrochemical detection is based on oxidation or reduction of a chemical compound to which a voltage has been applied. Electrons are either donated (oxidation) or accepted (reduction), which can be monitored as current. For both UV absorption and electrochemical detection, sensitivity for each individual nucleotide varies depending on the component base, but with molecules of sufficient length this bias is insignificant, and detection levels can be taken as a direct reflection of overall nucleic acid content.

Some embodiments of the invention include identifying molecules indirectly by detection of an associated label. A number of labels may be employed that provide a fluorescent signal for detection. If a sufficient quantity of a given species is generated in a reaction, and the mode of detection has sufficient sensitivity, then some fluorescent molecules may be incorporated into one or more of the primers used for amplification, generating a signal strength proportional to the concentration of DNA molecules. Several fluorescent moieties, including Alexa 350, Alexa 430, AMCA, BODIPY 630/650, BODIPY 650/665, BODIPY-FL, BODIPY-R6G, BODIPY-TMR, BODIPY-TRX, carboxyfluorescein, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red, are generally known in the art and routinely used for identification of discrete nucleic acid species, such as in sequencing reactions. Many of these dyes have emission spectra distinct from one another, enabling deconvolution of data from incompletely resolved samples into individual signals. This allows pooling of separate reactions that are each labeled with a different dye, increasing the throughput during analysis, as described in more detail below. Additional examples of suitable labels are described herein.

The signal strength obtained from fluorescent dyes can be enhanced through use of related compounds called energy transfer (ET) fluorescent dyes. After absorbing light, ET dyes have emission spectra that allow them to serve as “donors” to a secondary “acceptor” dye that will absorb the emitted light and emit a lower energy fluorescent signal. Use of these coupled-dye systems can significantly amplify fluorescent signal. Examples of ET dyes include the ABI PRISM BigDye terminators, recently commercialized by Perkin-Elmer Corporation (Foster City, Calif., USA) for applications in nucleic acid analysis. These chromophores incorporate the donor and acceptor dyes into a single molecule and an energy transfer linker couples a donor fluorescein to a dichlororhodamine acceptor dye, and the complex is attached, e.g., to a primer.

Fluorescent signals can also be generated by non-covalent intercalation of fluorescent dyes into nucleic acids after their synthesis and prior to separation. This type of signal will vary in intensity as a function of the length of the species being detected, and thus signal intensities must be normalized based on size. Several applicable dyes are known in the art, including, but not limited to, ethidium bromide and Vistra Green. Some intercalating dyes, such as YOYO or TOTO, bind so strongly that separate DNA molecules can each be bound with a different dye and then pooled, and the dyes will not exchange between DNA species. This enables mixing separately generated reactions in order to increase multiplexing during analysis.

Alternatively, technologies such as the use of nanocrystals as a fluorescent DNA label (Alivisatos, et al. (1996) Nature 382:609-11, which is incorporated by reference) can be employed in the methods of the present invention. Another method, described by Mazumder, et al. (Nucleic Acids Res. (1998) 26:1996-2000, which is incorporated by reference), describes hybridization of a labeled oligonucleotide probe to its target without physical separation from unhybridized probe. In this method, the probe is labeled with a chemiluminescent molecule that in the unbound form is destroyed by sodium sulfite treatment, but is protected in probes that have hybridized to target sequence.

In other embodiments, both electrochemical and infrared methods of detection can be amplified over the levels inherent to nucleic acid molecules through attachment of EC or IR labels. Their characteristics and use as labels are described in, for example, PCT publication WO 97/27327, which is incorporated by reference. Some preferred compounds that can serve as an IR label include an aromatic nitrile, aromatic alkynes, or aromatic azides. Numerous compounds can serve as an EC label; many are listed in PCT publication WO 97/27327.

Enzyme-linked reactions are also employed in the detecting step of the methods of the present invention. Enzyme-linked reactions theoretically yield an infinite signal, due to amplification of the signal by enzymatic activity. In this embodiment, an enzyme is linked to a secondary group that has a strong binding affinity to the molecule of interest. Following separation of the nucleic acid products, enzyme is bound via this affinity interaction. Nucleic acids are then detected by a chemical reaction catalyzed by the associated enzyme. Various coupling strategies are possible utilizing well-characterized interactions generally known in the art, such as those between biotin and avidin, an antibody and antigen, or a sugar and lectin. Various types of enzymes can be employed, generating colorimetric, fluorescent, chemiluminescent, phosphorescent, or other types of signals. As an illustration, a primer may be synthesized containing a biotin molecule. After amplification, amplicons are separated by size, and those made with the biotinylated primer are detected by binding with streptavidin that is covalently coupled to an enzyme, such as alkaline phosphatase. A subsequent chemical reaction is conducted, detecting bound enzyme by monitoring the reaction product. The secondary affinity group may also be coupled to an enzymatic substrate, which is detected by incubation with unbound enzyme. One of skill in the art can conceive of many possible variations on the different embodiments of detection methods described above.

In some embodiments, it may be desirable prior to detection to separate a subset of amplification products from other components in the reaction, including other products. Exploitation of known high-affinity biological interactions can provide a mechanism for physical capture. Some examples of high-affinity interactions include those between a hormone with its receptor, a sugar with a lectin, avidin and biotin, or an antigen with its antibody. After affinity capture, molecules are retrieved by cleavage, denaturation, or eluting with a competitor for binding, and then detected as usual by monitoring an associated label. In some embodiments, the binding interaction providing for capture may also serve as the mechanism of detection.

Furthermore, the size of an amplification product or products are optionally changed, or “shifted,” in order to better resolve the amplification products from other products prior to detection. For example, chemically cleavable primers can be used in the amplification reaction. In this embodiment, one or more of the primers used in amplification contains a chemical linkage that can be broken, generating two separate fragments from the primer. Cleavage is performed after the amplification reaction, removing a fixed number of nucleotides from the 5′ end of products made from that primer. Design and use of such primers is described in detail in, for example, PCT publication WO 96/37630, which is incorporated by reference.

### Data Analysis

For reliably classifying AML, for example, it is generally desirable to determine the expression of more than one of the markers described herein. As an exemplary criterion for the choice of markers, the statistical significance of markers as expressed in q or p values based on the concept of the false discovery rate is optionally determined. In doing so, a measure of statistical significance called the q value is associated with each tested feature. The q value is similar to the p value, except it is a measure of significance in terms of the false discovery rate rather than the false positive rate (see, e.g., Storey et al. (2003) Proc. Natl. Acad. Sci. 100:9440-5, which is incorporated by reference).

In some embodiments, the markers described herein have q-values of less than about 3E-06, typically less than about 1.5E-09, more typically less than about 1.5E-11, even more typically less than about 0.5E-20, and still more typically less than about 1.5E-30.

Of the markers described or referred to herein, the expression level of at least about two, typically of at least about ten, more typically of at least about 25, and even more typically of at least about 50 of these markers is determined as described herein or by another technique known to those of skill in the art. In some embodiments, for example, expression levels of one or more of the genes listed in Tables 1-13 are determined in a given sample. In certain embodiments, expression levels of each of these genes in a sample is determined and compared with expression levels detected in one or more reference cells. Furthermore, the International Publication No. WO 03/039443, which is incorporated by reference, discloses certain marker genes the expression levels of which are characteristic for certain leukemia. Certain of the markers and/or methods disclosed therein are optionally utilized in performing the methods described herein.

The level of the expression of a marker is indicative of the class of AML cell. The level of expression of a marker or group of markers is measured and is generally compared with the level of expression of the same marker or the same group of markers from other cells or samples. The comparison may be effected in an actual experiment or in silico. There is a meaningful difference in these levels of expression, e.g., when these expression levels (also referred to as expression pattern, expression signature, or expression profile) are measurably different. In some embodiments, the difference is typically at least about 5%, 10% or 20%, more typically at least about 50% or may even be as high as 75% or 100%. To further illustrate, the difference in the level of expression is optionally at least about 200%, i.e., two fold, at least about 500%, i.e., five fold, or at least about 1000%, i.e., 10 fold in some embodiments.

In certain embodiments, for example, the expression level of markers expressed lower in a first subtype than in at least one second subtype, which differs from the first subtype, is at least about 5%, 10% or 20%, more typically at least about 50% or may even be about 75% or about 100%, more typically at least about 10-fold, even more typically at least 50-fold, and still more typically at least about 100-fold lower in the first subtype. On the other hand, the expression level of markers expressed higher in a first subtype than in at least one second subtype, which differs from the first subtype, is at generally least about 5%, 10% or 20%, more generally at least about 50% or may even be about 75% or about 100%, more generally at least 10-fold, still more generally at least about 50-fold, and even more generally at least about 100-fold higher in the first subtype.

The classification accuracy of a given gene list for a set of microarray experiments is preferably estimated using Support Vector Machines (SVM), because there is evidence that SVM-based prediction slightly outperforms other classification techniques, such as k-Nearest Neighbors (k-NN). The LIBSVM software package version 2.36, for example, is optionally used (SVM-type: SVC, linear kernel (http://www.csie.ntu.edu.tw/-cj.1in/libsvrn/)). Machine learning algorithms are also described in, e.g., Brown et al. (2000) Proc. Natl. Acad. Sci., 97:262-267, Furey et al. (2000) Bioinformatics, 16:906-914, and Vapnik, Statistical Learning Theory, Wiley (1998), which are each incorporated by reference.

To further illustrate, the classification accuracy of a given gene list for a set of microarray experiments can be estimated using Support Vector Machines (SVM) as supervised learning techniques. Generally, SVMs are trained using differentially expressed genes, which were identified on a subset of the data and then this trained model is employed to assign new samples to those trained groups from a second and different data set. Differentially expressed genes are optionally identified, e.g., applying analysis of variance (ANOVA) and t-test-statistics (Welch t-test). Based on identified distinct gene expression signatures, respective training sets consisting of, e.g., ⅔ of cases and test sets with ⅓ of cases to assess classification accuracies can be designated. Assignment of cases to training and test sets is optionally randomized and balanced by diagnosis. Based on the training set, a Support Vector Machine (SVM) model can be built using this approach.

The apparent accuracy of prediction, i.e., the overall rate of correct predictions of the complete data set can be estimated by, e.g., 10-fold cross validation. This process typically includes dividing the data set into 10 approximately equally sized subsets, training an SVM-model for 9 subsets, and generating predictions for the remaining subset. This training and prediction process can be repeated 10 times to include predictions for each subset. Subsequently the data set can be split into a training set, consisting of two thirds of the samples, and a test set with the remaining one third. Apparent accuracy for the training set can also be estimated by 10fold cross validation (analogous to apparent accuracy for complete set). An SVM-model of the training set is optionally built to predict diagnosis in the independent test set, thereby estimating true accuracy of the prediction model. This prediction approach can be applied both for overall classification (multi-class) and binary classification (diagnosis X=>yes or no). For the latter, sensitivity and specificity are optionally calculated, as follows:

Sensitivity=(number of positive samples predicted)/(number of true positive)

Specificity=(number of negative samples predicted)/(number of true negatives).

### Systems for Gene Expression Analysis

The present invention also provides systems for analyzing gene expression. The system includes one or more probes that correspond to at least portions of genes or expression products thereof. The genes are selected from the markers listed in one or more of Tables 1-42. In some embodiments, for example, the probes are nucleic acids (e.g., oligonucleotides, cDNAs, cRNAs, etc.), whereas in other embodiments, the probes are biomolecules (e.g., antibodies, aptmers, etc.) designed to detect expression products of the genes (e.g., proteins or fragments thereof). In certain embodiments, the probes are arrayed on a solid support, whereas in others, they are provided in one or more containers, e.g., for assays performed in solution. The system also includes at least one reference data bank or database for correlating detected expression levels of polynucleotides and/or polypeptides in at least one target cell from a subject, which polynucleotides and/or polypeptides are targets of one or more of the probes, with the target cell being an AML cell. In some embodiments, the reference data bank is backed up on a computational data memory chip or other computer readable medium, which can be inserted in as well as removed from system of the present invention, e.g., like an interchangeable module, in order to use another data memory chip containing a different reference data bank. In certain embodiments, the systems also include detectors (e.g., spectrometers, etc.) that detect binding between the probes and targets. Other detectors are described further below. In addition, the systems also generally include at least one controller operably connected to the reference data bank and/or to the detector. In some embodiments, for example, the controller is integral with the reference data bank.

The systems of the present invention that include a desired reference data bank can be used in a way such that an unknown sample is, first, subjected to gene expression profiling, e.g., by microarray analysis in a manner as described herein or otherwise known to person skilled in the art, and the expression level data obtained by the analysis are, second, fed into the system and compared with the data of the reference data bank obtainable by the above method. For this purpose, the apparatus suitably contains a device for entering the expression level of the data, for example, a control panel such as a keyboard. The results, whether and how the data of the unknown sample fit into the reference data bank can be made visible on a monitor or display screen and, if desired, printed out on an incorporated of connected printer. Computer components are described further below.

In some embodiments, a system optionally further includes a thermal modulator operably connected to containers to modulate temperature in the containers (e.g., to effect thermocycling when target nucleic acids are amplified in the containers), and/or fluid transfer components (e.g., automated pipettors, etc.) that transfer fluid to and/or from the containers. Optionally, these systems also include robotic components for translocating solid supports, containers, and the like, and/or separation components (e.g., microfluidic devices, chromatography columns, etc.) for separating the products of amplification reactions from one another.

The invention further provides a computer or computer readable medium that includes a data set that comprises a plurality of character strings that correspond to a plurality of sequences (or subsequences thereof) that correspond to genes selected from, e.g., the list provided in Tables 1-42. Typically, the computer or computer readable medium further includes an automatic synthesizer coupled to an output of the computer or computer readable medium. The automatic synthesizer accepts instructions from the computer or computer readable medium, which instructions direct synthesis of, e.g., one or more probe nucleic acids that correspond to one or more character strings in the data set.

Detectors are structured to detect detectable signals produced, e.g., in or proximal to another component of the system (e.g., in container, on a solid support, etc.). Suitable signal detectors that are optionally utilized, or adapted for use, in these systems detect, e.g., fluorescence, phosphorescence, radioactivity, absorbance, refractive index, luminescence, or the like. Detectors optionally monitor one or a plurality of signals from upstream and/or downstream of the performance of, e.g., a given assay step. For example, the detector optionally monitors a plurality of optical signals, which correspond in position to “real time” results. Example detectors or sensors include photomultiplier tubes, CCD arrays, optical sensors, temperature sensors, pressure sensors, pH sensors, conductivity sensors, scanning detectors, or the like. Each of these as well as other types of sensors is optionally readily incorporated into the systems described herein. Optionally, the systems of the present invention include multiple detectors.

More specific exemplary detectors that are optionally utilized in these systems include, e.g., a resonance light scattering detector, an emission spectroscope, a fluorescence spectroscope, a phosphorescence spectroscope, a luminescence spectroscope, a spectrophotometer, a photometer, and the like. Various synthetic components are also utilized, or adapted for, use in the systems of the invention including, e.g., automated nucleic acid synthesizers, e.g., for synthesizing the oligonucleotides probes described herein. Detectors and synthetic components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), both of which are incorporated by reference.

The systems of the invention also typically include controllers that are operably connected to one or more components (e.g., detectors, synthetic components, thermal modulator, fluid transfer components, etc.) of the system to control operation of the components. More specifically, controllers are generally included either as separate or integral system components that are utilized, e.g., to receive data from detectors, to effect and/or regulate temperature in the containers, to effect and/or regulate fluid flow to or from selected containers, or the like. Controllers and/or other system components is/are optionally coupled to an appropriately programmed processor, computer, digital device, or other information appliance (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming, e.g., such as controlling fluid flow regulators in response to fluid weight data received from weight scales or the like.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer which is known to one of skill. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention. Software for performing, e.g., controlling temperature modulators and fluid flow regulators is optionally constructed by one of skill using a standard programming language such as Visual basic, Fortran, Basic, Java, or the like.

Reference data banks can be produced by, e.g., (a) compiling a gene expression profile of a patient sample by determining the expression level at least one marker selected from, e.g., those listed in one or more of Tables 1-42, and (b) classifying the gene expression profile using a machine learning algorithm. Exemplary machine learning algorithms are optionally selected from, e.g., Weighted Voting, K-Nearest Neighbors, Decision Tree Induction, Support Vector Machines (SVM), and Feed-Forward Neural Networks. In some embodiments, for example, the machine learning algorithm is an SVM, such as polynomial kernel, linear kernel, and Gaussian Radial Basis Function-kernel SVM models.

### Kits

The present invention also provides kits that include at least one probe as described herein for classifying AML. The kits also include instructions for correlating detected expression levels of polynucleotides and/or polypeptides in at least one target cell from a subject, which polynucleotides and/or polypeptides are targets of one or more of the probes, with the target cell being an AML cell. The invention also provides kits for providing prognostic information to subjects or patients diagnosed with AML according to the related methods described herein. Typically, the kits include suitable auxiliaries, such as buffers, enzymes, labeling compounds, and/or the like. In some embodiments, probes are attached to solid supports, e.g. the wells of microtiter plates, nitrocellulose membrane surfaces, glass surfaces, to particles in solution, etc. As another option, probes are provided free in solution in containers, e.g., for performing the methods of the invention in a solution phase. In certain embodiments, kits also contain at least one reference cell. For example, the reference can be a sample, a database, or the like. In some embodiments, the kit includes primers and other reagents for amplifying target nucleic acids. Typically, kits also include at least one container for packaging the probes, the set of instructions, and any other included components.

### EXAMPLES

It is understood that the examples and embodiments described herein are for illustrative purposes only and are not intended to limit the scope of the claimed invention. It is also understood that various modifications or changes in light the examples and embodiments described herein will be suggested to persons skilled in the art and are to be included within the spirit and purview of this application and scope of the appended claims.

### Example 1

### General Experimental Design and Results

CEBPA-Mutations in AML with Prognostically Intermediate Cytogenetics

Approximately 50% of acute myeloid leukemia (AML) have no karyotype changes or those with yet unknown prognostic significance. They are usually pooled together into the prognostically intermediate group.

This analysis assessed the role of CEBPA mutations within this AML subgroup. In total, 255 AML, 237 with normal and 18 with other intermediate risk group karyotypes were screened for CEBPA mutations by sequencing. The total incidence of CEBPA mutations was 51/255 (20%) ( 48/237 (20.3%) in the normal and 3/18 (16.7%) in the other karyotypes). Most of the patients showed an M1 (n=16), or M2 (n=25) morphology, but there were also some with FAB M0 (n=1), M4 (n=4), M5 (n=3), and M6 (n=2). CEBPA+ (i.e., having a CEBPA mutation) cases were younger as compared to the CEBPA− (i.e., lacking a CEBPA mutation) cases (54.7 vs. 60.0, p=0.023). Leukocyte and platelet counts were similar. Clinical follow up data were available for 191 (37 mutated, 154 non-mutated) patients. Overall survival (OS) and event-free survival (EFS) were significantly better in the patients with compared to those without CEBPA mutations (median 1092 vs. 259 days, p=0.0072; 375 vs. 218 days, p=0.0102, respectively). In addition, 18/42 (42.9%) of CEBPA+ cases had an FLT3-LM, 4/40 (10%) an FLT3-TKD, 4/41 (9.8%) an MLL-PTD, 3/34 (8.8%) an NRAS, 2/40 (5%) a KITD816 mutation. In four cases 2 additional mutations were detected: 1×FLT3-LM+KITD816, 1×FLT3-LM+FLT3-TKD, and 2×MLL-PTD+FLT3-LM. The favorable prognostic impact of CEBPA mutations was not affected by additional mutations.

In addition, 22 of the CEBPA+ cases were analyzed by microarray analysis using the U133A+B array set (Affymetrix, Inc., Santa Clara, Calif., USA) and compared to the expression profile of 131 CEBPA− normal karyotype AML, as well as to 204 AML characterized by the reciprocal translocations t(15;17) (n=43), t(8;21) (n=36), inv(16) (n=48), t(11q23) (n=50), inv(3) (n=27). The discrimination of CEBPA+ cases and reciprocal translocations revealed a classification accuracy of 94.7% with 75% sensitivity and 98.5% specificity. However, the CEPBA+ cases did not show a specific expression pattern within the total group with normal karyotype and could not be discriminated from CEBPA− cases. By use of PCA and hierarchical cluster analysis it was obvious that the CEBPA+ cases separated into two domains. One subcluster (cluster 1) was distributed among the cases with CEBPA− normal karyotype AML. A second cluster (cluster 2) was very close to the t(8;21) cases. Accordingly, cases of cluster 2 similar to t(8;21) and in contrast to cluster 1 highly expressed MPO and had low expression of HOXA3, HOXA7, HOXA9, HOXB4, HOXB6, and PBX3. Using the top 100 differentially expressed genes and applying 100 runs of SVM with ⅔ of samples being randomly selected as training set and ⅓ as test set samples, groups A and B could be classified with an overall accuracy of 100% (sensitivity 100% and specificity 100%). A detailed analysis of the two subclusters showed that all 8 cases of cluster 1 revealed mutations in the TAD2 domain of CEBPA and 6 of these had an FLT3-LM in addition. In contrast, 12/14 cases of cluster 2 had mutations that lead to an N-terminal stop and only 2 had an FLT3-LM. Thus these two subclusters have biological differences that may explain the different gene expression patterns. Despite the different functional consequences of the mutations in the two CEBPA-clusters no differences with respect to FAB type and prognosis were found between cluster 1 and 2.

Analysis of Molecular Markers in the Prognostically Intermediate Karyotype Group in AML

Acute myeloid leukemia (AML) can be divided into prognostically different subgroups based on chromosomal aberrations. However, more than 50% of AML have no karyotype changes or those with yet unknown prognostic significance and they are usually pooled together into the prognostically intermediate karyotype group (1-AML).

This analysis approached the subclassification of this large AML group by using molecular markers. Six genes were screened for mutations and analyzed for their prognostic significance in comparison to cases without the respective mutation. Results of this analysis are given in Table 14, below. Significant unfavorable impact on overall survival (OS) was shown for the MLL-PTD in the total group and for AML1 mutations in FAB M0. Event-free survival (EFS) and relapse-free survival (RFS) was adversely affected by the FLT3-LM and EFS in AML1 mutated cases. In contrast CEBPA mutations disclose a favorable subgroup. Molecular mutations are not mutually exclusive. At least one additional mutation was observed in all possible combinations in 1.1% to 34.7% (mean 10.9%). The most frequent combinations are MLL-PTD+FLT3-LM in 34.7% of all MLL-PTD+ cases and CEBPA+FLT3-LM in 34.4% of all CEBPA+ cases. In contrast, double mutations of FLT3 or combinations of FLT3 or KIT with NRAS are rare (1.1%-3.6%), suggesting a better cooperativity of CEBPA and MLL-PTD with FLT3-LM. For all combinations an effect on prognosis could not be shown in addition to those given in Table 14. Three mutations were detected in 6 cases and again all of the possible genes were involved at least once. In only one third of all I-AML patients none of the analyzed mutations was detected. A two step hypothesis has recently been postulated for AML with fusion transcripts. The presented data support a two or maybe multistep theory for mutagenesis in AML with normal karyotype. Molecular mutations may have less transforming capacity, so that more than two mutations have to be accumulated. The pattern of the detected mutations suggests CEBPA and MLL-PTD to be type II mutations (differentiation) whereas FLT3, KIT, and RAS have previously postulated to be type I mutations (proliferation).

In addition, gene expression studies were performed in 228 I-AML positive for one or more of the mutations. All of the different mutation groups did not reveal distinct individual expression patterns. This suggests that specific pathways may be involved in the normal karyotype AML that are triggered redundantly by different gene mutations.

TABLE 14
PROGNOSTIC SIGNIFICANCE OF GENE MUTATIONS
COMPARED TO THE GROUP TESTED NEGATIVE FOR THIS
MUTATION IN THE PROGNOSTICALLY INTERMEDIATE GROUP

| | AML1 (M0) | CEBPA | KIT | FLT3-LM | FLT-TKD | MLL-PTD | NRAS |
|---|---|---|---|---|---|---|---|
| analyzed | 80 | 191 | 676 | 1003 | 847 | 1024 | 718 |
| +/− cases | 13/67 | 37/154 | 12/664 | 317/686 | 62/785 | 96/928 | 71/647 |
| frequency | 16.2% | 19.4% | 1.8% | 31.5% | 7.3% | 9.4% | 9.9% |
| OS (p =) | 0.0416# | 0.0072* | 0.6229 | 0.1834 | 0.9327 | 0.0193# | 0.4042 |
| EFS (p =) | 0.0345# | 0.0102* | 0.3186 | 0.0124# | 0.9898 | 0.1226 | 0.7637 |
| RFS (p =) | — | 0.0228* | 0.4143 | 0.0012# | 0.4074 | 0.6700 | 0.7310 |

*favorable,
#unfavorable

Acute myeloid leukemia (AML) is a heterogeneous group of diseases with varying clinical outcomes. So far the karyotype of the leukemic blasts as well as molecular genetic abnormalities (both abnormalities on the genomic level) have been proven to be strong prognostic markers. However, even in genetically well-defined subgroups clinical outcome is not uniform and a large proportion of AML shows genetic abnormalities of yet unknown prognostic significance.

The analyses described in this example addressed the question whether gene expression profiles are associated with clinical outcome independent of the known genomic abnormalities. More specifically, gene expression analyses were performed using Affymetrix U133A+B oligonucleotide microarrays in a total of 403 AML treated uniformly in the AMLCG studies. This cohort was divided randomly into a training set (n=269) and a test set (n=134). The training set included 18 cases with t(15;17), 22 cases with t(8;21), 29 cases with inv(16), 14 cases with 11q23/MLL-rearrangement, 19 with complex aberrant karyotype and 167 cases with normal karyotype or other chromosome aberrations. The respective data for the test set were: 10 t(15;17), 8 t(8;21), 11 inv(16), 8 11q23/MLL, 19 cases with complex aberrant karyotype and 78 with normal karyotype or other chromosome aberrations. Based on the clinical outcome the training cohort was divided into 4 equally large subgroups. Support vector machines (SVM) where trained with the training set and classified the cases of the test set with the respective most discriminating genes. Next a Kaplan-Meier analysis was performed with the test set cases assigned to prognostic groups 1 to 4 according to SVM classification. Based on the expression level of 100 genes group 1 showed an overall survival rate of 57% at 3 years. 31 of 134 (23%) patients were assigned to this favorable subgroup. They belonged to the following cytogenetic subgroups: t(15;17) n=6, t(8;21) n=4, inv(16) n=3, 11q23/MLL n=4, complex aberrant karyotype n=1 and normal karyotype or other chromosome aberration n=13. The overall survival rate of groups 2, 3, and 4 did not differ significantly (17%, 21%, and 19% at 3 years). Among the genes highly expressed in the favorable group were MPO and the transcription factor ATBF1, which regulates CCND1. The unfavorable groups were characterized by a higher expression of the transcription factors ETS2, RUNX1, TCF4, and FOXC1. Interestingly, 10 of the top 40 differentially expressed genes are involved in the TP53-CMYC-pathway with a higher expression of 9 of these in the unfavorable groups (SFRS1, TPD52, NRIP1, TFPI, UBL1, REC8L1, HSF2, ETS2 and RUNX1). See, Tables 1-3. In conclusion, gene expression profiling leads to the identification of prognostically important alterations of molecular pathways which have not yet been accounted for by use of cytogenetics. This approach is can be utilized in, e.g., optimizing therapy for patients with AML.

Balanced chromosomal rearrangements leading to fusion genes on the molecular level define distinct biological subsets in AML. The four balanced rearrangements (t(15;17), t(8;21), inv(16), and 11q23/MLL) show a close correlation to cytomorphology and gene expression patterns. In this example, the focus was on seven AML with t(8;16) (p11;p13). This translocation is rare (7/3515 cases in own cohort). It is more frequently found in therapy-related AML than in de novo AML (3/258 t-AML, and 4/3287 de novo, p=0.0003). Cytomorphologically, AML with t(8;16) is characterized by striking features: in all 7 cases the positively for myeloperoxidase on bone marrow smears was >70% and intriguingly, in parallel >80% of blast cells stained strongly positive for non-specific esterase (NSE) in all cases. Thus, these cases could not be classified according to FAB categories. These data suggested that AML-t(8;16) arise from a very early stem cell with both myeloid and monoblastic potential. Furthermore, erythrophagocytosis was detected in 6/7 cases that was described as specific feature in AML with t(8;16). Four patients had chromosomal aberrations in addition to t(8;16), 3 of these were t-AML all showing aberrations of 7q. Survival was poor with 0, 1, 1, 2, 20 and 18+ (after alloBMT) months, one lost to follow-up, respectively. Gene expression patterns were analyzed in 4 cases (Affymetrix U133A+B). First, t(8;16) AML was compared with 46 AML FAB M1, 41 M4, 9 M5a, and 16 M5b, all with normal karyotypes. Hierachical clustering and principal component analyses (PCA) revealed that t(8;16) AML were intercalating with FAB M4 and M5b and did not cluster near to M1. Thus, monocytic characteristics influence the gene expression pattern stronger than myeloid. Next, the t(8;16) AML was compared with the 4 other balanced subtypes according to the WHO classification (t(15;17): 43; t(8;21): 40; inv(16): 49;11q23/MLL-rearrangements: 50). Using support vector machines, the overall accuracy for correct subgroup assignment was 97.3% (10-fold CV), and 96.8% (⅔ training and ⅓ test set, 100 runs). In PCA and hierarchical cluster analysis, the t(8;16) was grouped in the vicinity of the 11q23 cases. However, in a pairwise comparison these two subgroups could be discriminated with an accuracy of 94.4% (10-fold CV). Genes with a specific expression in AML-t(8;16) were further investigated in pathway analyses (Ingenuity Systems (Mountain View, Calif., USA)). 15 of the top 100 genes associated with AML-t(8;16) were involved in the CMYC-pathway with up regulation or higher expression of BCOR, COXB5, CDK10, FLI1, HNRPA2B1, NSEP1, PDIP38, RAD50, SUPT5H, TLR2 and USP33, and down regulation or lower expression of ERG, GATA2, NCOR2 and RPS20. CEBP beta, known to play a role in myelomonocytic differentiation, was also up-regulated in t(8;16)-AML. Ten additional genes out of the 100 top differentially expressed genes were also involved in this pathway with up-regulation of DDB2, HIST1H3D, NSAP1, PTPNS1, RAN, USP4, TRIM8, and ZNF278 and down regulation of KIT and MBD2. In conclusion, AML with t(8;16) is a specific subtype of AML with unique characteristics in morphology and gene expression patterns. It is more frequently found in t-AML, outcome is inferior in comparison to other AML with balanced translocations. Due to its unique features, it is a candidate for inclusion into the WHO classification as a specific entity.

Among the aims of this study was to analyze the impact of trisomy 8 on the expression of genes located on chromosome 8 in different AML subgroups. Therefore, gene expression analyses were performed in a total of 567 AML cases using Affymetrix U133A+B oligonucleotide microarrays (Affymetrix, Inc., Santa Clara, Calif., USA). The following 14 subgroups were analyzed: +8 sole (n=19), +8 within a complex aberrant karyotype (n=11), +8 with t(115;17) (n=7), +8 and inv(16) (n=3), +8 with t(8;21) (n=3), +8 and 11q23/MLL (n=8), and +8 with other abnormalities (n=10). These were compared to 200 AML with normal karyotype and the following subgroups without trisomy 8: complex aberrant karyotype (n=73), t(15;17) (n=36), inv(16) (n=46), t(8;21) (n=37), 11q23/MLL (n=37), and other abnormalities (n=77). In total, 1188 probe sets covered sequences located on chromosome 8 representing 580 genes. A significant higher mean expression of all genes located on chromosome 8 was observed in subgroups with +8 in comparison to their respective control groups (for all comparisons, p<0.05). Significantly higher expressed genes in groups with +8 in comparison to the respective groups without +8 were identified in all comparisons. The number of identified genes ranged from 40 in 11q23/MLL to 326 in trisomy 8 sole vs. normal. There was no common gene significantly overexpressed in all comparisons. Three genes (TRAM1, CHPPR, MGC40214) showed a significantly higher expression in 5 out of 7 comparisons. Between 19 and 107 genes with an exclusive overexpression in trisomy 8 cases in only one subtype comparison were identified.

In addition, class prediction was performed using support vector machines (SVM) including all probe sets on the arrays. In one approach, all 14 different subgroups were analyzed as one class each. Only 3 out of 61 cases with trisomy 8 were assigned into their correct subclass, while 40 cases were assigned to their corresponding genetic subclass without trisomy 8. In a second approach only two classes were defined: all cases with trisomy 8 combined vs. all cases without trisomy 8. Only 26 out of 61 (42.6%) with trisomy 8 were identified correctly underlining the fact that no distinct gene expression pattern is associated with trisomy 8 in general. Performing SVM only with genes located on chromosome 8 did not improve the correct assignment of cases with trisomy 8 overall. Only cases with trisomy 8 sole were correctly predicted in 58% as compared to 11% in SVM using all genes.

To further illustrate, the 50 most differentially expressed genes between AML with and without trisomy 8 are listed in Table 19. The expression of genes was compared between the mentioned subtypes characterized by a specific karyotype pattern and AML with the same specific karyotype with trisomy 8 in addition. The most differentially expressed genes are specified in Tables 21, 23, 25, 27, 29, 31, and 33 (specific karyotype patterns are indicated in the respective Tables). The most differentially genes taking into account only genes located on chromosome 8 for the respective comparisons are listed in the respective Tables 22, 24, 26, 28, 30, 32, and 34. In particular, differentially expressed genes between t(8;21) and t(8;21) with trisomy 8 are listed in Tables 20 and 21; differentially expressed genes between t(15;17) and t(15;17) with trisomy 8 are listed in Tables 23 and 24; differentially expressed genes between inv(16) and inv(16) with trisomy 8 are listed in Tables 25 and 26; differentially expressed genes between 11q23/MLL and 11q23/MLL with trisomy 8 are listed in Tables 27 and 28; differentially expressed genes between normal karyotype and normal karyotype with trisomy 8 are listed in Tables 29 and 30; differentially expressed genes between other abnormalities and the other abnormalities with trisomy 8 are listed in Tables 31 and 32; and differentially expressed genes between complex aberrant karyotype and the complex aberrant karyotype with trisomy 8 are listed in Tables 33 and 34.

In conclusion, overall the gain of chromosome 8 leads to a higher expression of genes located on chromosome 8. However, no consistent pattern of genes was identified which shows a higher expression in all AML subtypes with trisomy 8. This data suggest that the higher expression of genes located on chromosome 8 only in part is directly related to a gene dosage effect. Trisomy 8 may rather provide a platform for a higher expression of chromosome 8 genes which are specifically upregulated by accompanying genetic abnormalities in the respective AML subtypes (Tables IV, VI, VII, X, XII, XIV, XVI). Therefore, trisomy 8 does not seem to be an abnormality determining specific disease characteristics such as the well known primary aberrations (t(8;21), inv(16), t(15;17), MLL/11q23) but rather a disease modulating secondary event in addition to primary cytogenetic or molecular genetic aberrations.

MDS and AML are discriminated by percentages of blasts in the bone marrow (BM) according to the FAB as well as to the WHO classification. However, thresholds are arbitrary and demonstrate only a limited reproducibility in interlaboratory testings. Thus, other parameters have been assessed to discriminate these entities with respect to diagnosis and prognosis. In particular, in the majority of cases common karyotype aberrations have been observed between MDS and AML, which have a higher prognostic impact than blast percentages.

In this example, gene expression profiling (U133A+B, Affymetrix) was applied in 70 MDS and 238 AML cases. In accordance with the WHO classification, cases with balanced translocations (i.e. t(8;21), t(15;17), inv(16), or 11q23), which are classified as AML irrespective of BM blast percentage, were excluded. First, the identity of genes of which the expression correlated to blast count (Spearman correlation) was sought. Out of the top 50 genes this analysis revealed only the FLT3 gene which showed a higher expression in cases with high blast count (e.g. AML), while 12 genes with a higher expression in cases with lower blast counts (e.g. MDS) were identified (ANXA3, ARG1, CAMP, CD24, CEACAM1, CEACAM6, CEACAM8, CRISP3, KIAA0922, LCN2, MMP9, STOM). Most of the latter genes are expressed in mature granulocytes and are involved in differentiation and apoptosis (see, e.g., more genes listed in Table 25). In a second step, class prediction was performed using support vector machines (SVM) to separate MDS and AML according to blast percentages as defined in the WHO classification (<5%: RA and 5q-syndrome; 5-9%: RAEB-1;10-19%: RAEB-2; >19% AML). Using 10-fold cross validation and support vector machines the overall prediction accuracy was only 80% (see, e.g., the genes listed in Table 36). More specifically, 230/238 AML cases were correctly assigned to the AML group while 8 cases were classified as MDS RAEB-2. However, none of the RA, 5q-syndrome and RAEB-1 cases were correctly assigned to their groups, respectively, but were either classified as AML or RAEB-2. Furthermore, only 16 of 38 RAEB-2 cases were correctly predicted, while the 20 remaining cases were assigned to the AML group. Thus, no clear gene expression patterns were identified which correlated with AML and MDS subtypes according to WHO classification.

Taking the common genetic background observed in MDS and AML into account, both entities were categorized in a third step according to cytogenetics and classified based on their gene expression profiles. In order to assess the impact of the common genetic background, the largest cytogenetically defined subgroups were compared to each other, i.e. AML and MDS with normal karyotype and with complex aberrant karyotype. Intriguingly, while correct classification of AML or MDS was found in 91%, classification into the correct cytogenetic groups was achieved in 95%. Consequently, all cases were divided into the two groups, complex aberrant karyotype (n=60) and other or no aberrations (n=248) irrespective of AML or MDS. A classification into these groups also yielded an accuracy of 93% (see, e.g., the genes listed in Table 37).

The data from these analyses suggests that gene expression profiling reveals the biology of MDS or AML to highly correlate with cytogenetics and less with the percentages of BM blasts. These results strengthen the need for a revision of the current MDS and AML classification centering now genetic abnormalities, which may also be used for clinical decisions.

To clarify the genetic background and to improve prognostication in AML-NK, gene expression profiles in 205 patients with untreated and newly diagnosed AML-NK were analyzed. Samples were comprehensively characterized by cytomorphology, immunophenotyping, cytogenetics, and molecular genetics. For expression profiling, samples were hybridized to both U133A and U133B microarrays (Affymetrix, Inc., Santa Clara, Calif., USA). To identify genetically defined subgroups, an unsupervised principal component analysis (PCA) was performed applying all 34023 probe sets from both arrays that were expressed in at least one of the analyzed samples. While the majority of cases (n=162, 79%; Group A) clustered together, a subgroup comprising 43 (21%) cases was identified (Group B) which formed a distinct cluster. The analysis of known genetic markers (length mutations and point mutations of FLT3, partial tandem duplications of MLL, mutations of CEBPA, NRAS, or CKIT) did not reveal differences between was performed Groups A and B. Significant differences were found, however, in their phenotypes. There were more cases with monocytic leukemias in group F (84% vs. 20%, p<0.001) and the expression levels of CD4, CD56, CD65, CD15, CD14, CD64, CD11b, CD36, CD135, CD87, and CD116 were higher while those of MPO, CD34, and CD117 were lower (p<0.05 for all).

To identify the genetic background of differences, samples from Groups A and B were compared using a supervised approach. Using the top 100 differentially expressed genes and applying SVM with a 10-fold cross validation approach samples could be classified to Groups A and B with an accuracy of 97.6% which was confirmed applying 100 runs of SVM with ⅔ of samples being randomly selected as training set and ⅓ as test set (median accuracy, 97.1%, range, 93.4% to 100%). Ingenuity software was used to identify genetic pathways differentially regulated between both groups. Most strikingly, CD14 was higher expressed (fold-change (fc), 10.6) and WT1 and MYCN were lower expressed (fc, 3.7 and 4.4) in Group B. Also higher expressed was HCK (fc, 4.3) encoding a protein-tyrosine kinase which phosphorylates STAT3. Since phosphorylated STAT3 stimulates proliferation this may confer higher chemosensitivity and result in a better prognosis. The lower expression of HCK in Group A cases may be due to the higher expression of SPTBN1 (fc, 3.4) which also has been shown to increase the transcription of C-FOS and to possibly reveal antiapoptotic effects.

To assess the clinical importance of the newly identified subgroups of AML-NK event-tree survival (EFS) and overall survival (OS) were compared. All patients were uniformly treated within the German AMLCG trials. Group B had a significantly better median EFS (13.3 vs. 7.0 months, p=0.0143) which was independent of the impact of age. In addition, there was a trend for a better OS in Group B (13.3 vs. 9.5 months, n.s.).

In conclusion, the identification of a biologically defined and clinically relevant subgroup of AML-NK has been accomplished by use of gene expression profiling based on differences in regulations of genetic pathways involving proliferation and apoptosis.

Deletions of the long arm of chromosome 5 occur either as the sole karyotype abnormality in MDS and AML or as part of a complex aberrant karyotype. One objective of this study was to analyze the impact of the 5q deletion on the expression levels of genes located on chromosome 5q in AML and MDS. Therefore, gene expression analysis was performed in 344 AML and MDS cases using Affymetrix U133A+B oligonucleotide microarrays. The following subgroups were analyzed: AML with sole 5q deletion (n=7), AML with complex aberrant karyotype (n=83), MDS with sole 5q deletion (n=9), and MDS with complex aberrant karyotype (n=9). These were compared to 200 AML and 36 MDS with normal karyotype. In total, 1313 probe sets representing 603 genes cover sequences located on the long arm of chromosome 5. Overall a significant lower mean expression of all genes located on the long arm of chromosome 5 was observed in subgroups with 5q deletion in comparison to their respective control groups (for all comparisons, p<0.05). 36 genes showed a significantly lower expression in all comparisons. These genes are involved in a variety of different biological processes such as signal transduction (CSNK1A1, DAMS), cell cycle regulation (HDAC3, PFDN1) and regulation of transcription (CNOT8).

In addition, class prediction was performed using support vector machines (SVM). In one approach, all 6 different subgroups were analyzed as one class each. While AML and MDS with normal karyotype as well as AML with complex aberrant karyotype were correctly predicted with high accuracies (97%, 81%, and 92%, respectively) AML and MDS with 5q-sole and MDS with complex aberrant karyotype were frequently misclassified as AML with complex aberrant karyotype. In a second approach, only two classes were defined: all cases with 5q deletion combined vs. all cases without 5q deletion. 102 out of 108 cases (94%) with 5q deletion were identified correctly supporting the fact that a distinct gene expression pattern is associated with 5q deletion in general. Performing SVM only with genes located on the long arm of chromosome 5 also resulted in a correct prediction of 92 of 108 (85%) stressing the importance of the expression of genes located on chromosome 5 for these AML and MDS subtypes. The top 100 differentially expressed probe sets between cases with and without 5q deletion represented 74 different annotated genes of which 23 are located on the long arm of chromosome 5. They are involved in a variety of different biological functions such as DNA repair (POLE, RAD21, RAD23B), regulation of transcription (ZNF75A, AF020591, MLLT3, HOXB6), protein biosynthesis (UPF2, TINP1, RPL12, RPL14, RPL15) cell cycle control (GMNN, CSPG6, PFDN1) and signal transduction (HINT1, STK24, APP, CAMLG). 10 of the top 74 genes associated with 5q deletion were involved in the CMYC-pathway with upregulation of RAD21, RAD23B, GMMN, CSPG6, APP, POLE STK24 and STAG2, and downregulation of ACTA2, and RPL12. Ten other genes out of the 74 top differentially expressed genes were involved in the TP53 pathway with upregulation of H1F0, PTPN11 and TAF2 and downregulation of DF, UBE2D2, EEF1A1, IGBP1, PPP2CA, EIF2S3, and NACA.

In conclusion, loss of parts of the long arm of chromosome 5 leads to a lower expression of genes located on the long arm of chromosome 5. A specific pattern of functionally related genes was identified which shows a lower expression in AML and MDS subtypes with 5q deletion.

### Example 2

### General Materials, Methods and Definitions of Functional Annotations

The methods section contains both information on statistical analyses used for identification of differentially expressed genes and detailed annotation data of identified microarray probe sets.

### Affymetrix Probeset Annotation

All annotation data of GeneChip® arrays are extracted from the NetAffx™ Analysis Center (internet website: www.affymetrix.com). Files for U133 set arrays, including U133A and U133B microarrays are derived from the June 2003 release. The original publication refers to: Liu et al. (2003) “NetAffx: Affymetrix probe sets and annotations,” Nucleic Acids Res. 31(1):82-6, which is incorporated by reference.

The sequence data are omitted due to their large size, and because they do not change, whereas the annotation data are updated periodically, for example new information on chromosomal location and functional annotation of the respective gene products. Sequence data are available to download in the NetAffx Download Center on the world wide web at affymetrix.com.

### Data Fields

In the following section, the content of each field of the data files is described. Microarray probe sets, for example, found to be differentially expressed between different types of leukemia samples are further described by additional information. The fields are of the following types:
1. GeneChip Array Information
2. Probe Design Information
3. Public Domain and Genomic References

1. GeneChip Array Information
HG-U133 ProbeSet_ID:
HG-U133 ProbeSet_ID describes the probe set identifier. Examples are: 200007_at 200011_s_at,200012_x_at.

Sequence Type

The Sequence Type indicates whether the sequence is an Exemplar, Consensus or Control sequence. An Exemplar is a single nucleotide sequence taken directly from a public database. This sequence could be an mRNA or an expressed sequence tag (EST). A Consensus sequence is a nucleotide sequence assembled by Affymetrix, based on one or more sequence taken from a public database.

Transcript ID:

The cluster identification number with a sub-cluster identifier appended.

Sequence Derived From:

The accession number of the single sequence, or representative sequence on which the probe set is based. Refer to the “Sequence Source” field to determine the database used.

Sequence ID:

For Exemplar sequences: Public accession number or GenBank identifier. For Consensus sequences: Affymetrix identification number or public accession number.

Sequence Source

The database from which the sequence used to design this probe set was taken. Examples are: GenBank®, RefSeq, UniGene, TIGR (annotations from The Institute for Genomic Research).

2. Public Domain and Genomic References

Most of the data in this section is from the LocusLink and UniGene databases, and are annotations of the reference sequence on which the probe set is modeled.

Gene Symbol and Title:

A gene symbol and a short title, when one is available. Such symbols are assigned by different organizations for different species. Affymetrix annotational data comes from the UniGene record. There is no indication which species-specific databank was used, but some of the possibilities include for example HUGO: The Human Genome Organization.

MapLocation:

The map location describes the chromosomal location when one is available.

Unigene Accession:

UniGene accession number and cluster type. Cluster type can be “full length” or “est”, or “---” if unknown.

LocusLink:

This information represents the LocusLink accession number.

Full Length Ref. Sequences

Indicates the references to multiple sequences in RefSeq. The field contains the ID and description for each entry, and there can be multiple entries per probeSet.

### Example 3

### Sample Preparation, Processing and Data Analysis

### Method 1:

Microarray analyses were performed utilizing the GeneChip® System (Affymetrix, Santa Clara, USA). Hybridization target preparations were performed according to recommended protocols (Affymetrix Technical Manual). More specifically, at time of diagnosis, mononuclear cells were purified by Ficoll-Hypaque density centrifugation. They had been lysed immediately in RLT buffer (Qiagen, Hilden, Germany), frozen, and stored at −80° C. from 1 week to 38 months. For gene expression profiling cell lysates of the leukemia samples were thawed, homogenized (QIAshredder, Qiagen), and total RNA was extracted (RNeasy Mini Kit, Qiagen). Subsequently, 5-10 μg total RNA isolated from 1×10⁷ cells was used as starting material for cDNA synthesis with oligo[(dT)₂₄T7promotor]₆₅ primer (cDNA Synthesis System, Roche Applied Science, Mannheim, Germany). cDNA products were purified by phenol/chloroform/IAA extraction (Ambion, Austin, Tex., USA) and acetate/ethanol-precipitated overnight. For detection of the hybridized target nucleic acid biotin-labeled ribonucleotides were incorporated during the following in vitro transcription reaction (Enzo BioArray HighYield RNA Transcript Labeling Kit, Enzo Diagnostics). After quantification by spectrophotometric measurements and 260/280 absorbance values assessment for quality control of the purified cRNA (RNeasy Mini Kit, Qiagen), 15 μg cRNA was fragmented by alkaline treatment (200 mM Tris-acetate, pH 8.2/500 mM potassium acetate/150 mM magnesium acetate) and added to the hybridization cocktail sufficient for five hybridizations on standard GeneChip® microarrays (300 μL final volume). Washing and staining of the probe arrays was performed according to the recommended Fluidics Station protocol (EukGE-WS2v4). Affymetrix Microarray Suite software (version 5.0.1) extracted fluorescence signal intensities from each feature on the microarrays as detected by confocal laser scanning according to the manufacturer's recommendations.

Expression analysis quality assessment parameters included visual array inspection of the scanned image for the presence of image artifacts and correct grid alignment for the identification of distinct probe cells as well as both low 3′/5′ ratio of housekeeping controls (mean: 1.90 for GAPDH) and high percentage of detection calls (mean: 46.3% present called genes). The 3′ to 5′ ratio of GAPDH probesets can be used to assess RNA sample and assay quality. Signal values of the 3′ probe sets for GAPDH are compared to the Signal values of the corresponding 5′ probe set. The ratio of the 3′ probe set to the 5′ probe set is generally no more than 3.0. A high 3′ to 5′ ratio may indicate degraded RNA or inefficient synthesis of ds cDNA or biotinylated cRNA (GeneChip Expression Analysis Technical Manual, www.affymetrix.com). Detection calls are used to determine whether the transcript of a gene is detected (present) or undetected (absent) and were calculated using default parameters of the Microarray Analysis Suite MAS 5.0 software package.

Method 2:

Bone marrow (BM) aspirates are taken at the time of the initial diagnostic biopsy and remaining material is immediately lysed in RLT buffer (Qiagen), frozen and stored at −80° C. until preparation for gene expression analysis. For microarray analysis the GeneChip® System (Affymetrix, Santa Clara, Calif., USA) is used. The targets for GeneChip® analysis are prepared according to the current Expression Analysis. Briefly, frozen lysates of the leukemia samples are thawed, homogenized (QIAshredder, Qiagen) and total RNA extracted (RNeasy Mini Kit, Qiagen). Normally 10 μg total RNA isolated from 1×10⁷ cells is used as starting material in the subsequent cDNA-Synthesis using Oligo-dT-T7-Promotor Primer (cDNA synthesis Kit, Roche Molecular Biochemicals). The cDNA is purified by phenol-chloroform extraction and precipitated with 100% Ethanol overnight. For detection of the hybridized target nucleic acid biotin-labeled ribonucleotides are incorporated during the in vitro transcription reaction (Enzo BioArray™ High Yield RNA Transcript Labeling Kit, ENZO). After quantification of the purified cRNA (RNeasy Mini Kit, Qiagen), 15 μg are fragmented by alkaline treatment (200 mM Tris-acetate, pH 8.2, 500 mM potassium acetate, 150 mM magnesium acetate) and added to the hybridization cocktail sufficient for 5 hybridizations on standard GeneChip® microarrays. Before expression profiling Test3 Probe Arrays (Affymetrix) are chosen for monitoring of the integrity of the cRNA. Only labeled cRNA-cocktails which show a ratio of the measured intensity of the 3′ to the 5′ end of the GAPDH gene less than 3.0 are selected for subsequent hybridization on HG-U133 probe arrays (Affymetrix). Washing and staining the Probe arrays is performed as described (see, Affymetrix-Original-Literature (LOCKHART und LIPSHUTZ). The Affymetrix software (Microarray Suite, Version 4.0.1) extracted fluorescence intensities from each element on the arrays as detected by confocal laser scanning according to the manufacturers recommendations.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

TABLE 1
genes higher expressed in CEBPA than in reciprocal

| | | | | | | | Sequence | |
|---|---|---|---|---|---|---|---|---|
| | | | | | | | Derived | |
| # | affy id | HUGO name | Title | MapLocation | Sequence Type | Transcript ID | From | Sequence ID |
| 1 | 232424_at | PRDM16 | PR domain containing 16 | 1p36.23-p33 | Consensussequence | Hs.302022.1 | AI623202 | Hs.302022.1.S1 |
| 2 | 239791_at | | Homo sapiens, clone | | Consensussequence | Hs.269918.1 | AI125255 | Hs.269918.1.A1 |
| | | | MGC: 10077 IMAGE: | | | | | |
| | | | 3896690, mRNA, | | | | | |
| | | | complete cds | | | | | |
| 3 | 228904_at | | ESTs | | Consensussequence | Hs.156044.0 | AW510657 | Hs.156044.0 |
| 4 | 205366_s_at | HOXB6 | homeo box B6 | 17q21.3 | Exemplarsequence | Hs.98428.0 | NM_018952.1 | g9506792 |
| 5 | 210215_at | TFR2 | transferrin receptor 2 | 7q22 | Exemplarsequence | Hs.63758.1 | AF067864.1 | g5596369 |
| 6 | 235438_at | | ESTs | | Consensussequence | Hs.146226.0 | AW162011 | Hs.146226.0_RC |

| | Sequence | | | | |
|---|---|---|---|---|---|
| # | Source | Unigene_Accession | Cluster_Type | LocusLink | Full_Length_Reference_Seq |
| 1 | GenBank | Hs.302022 | fulllength | 63976 | NM_022114; PR domain containing 16 |
| 2 | GenBank | Hs.183096 | fulllength | | |
| 3 | GenBank | Hs.156044 | est | | |
| 4 | RefSeq | Hs.98428 | fulllength | 3216 | NM_018952; homeo box B6 isoform 1 |
| | | | | | NM_156036; homeo box B6 isoform 2 |
| | | | | | NM_156037; homeo box B6 isoform 1 |
| 5 | GenBank | Hs.63758 | fulllength | 7036 | NM_003227; transferrin receptor 2 |
| 6 | GenBank | Hs.445509 | est | | |

TABLE 2
genes lower expressed in CEBPA than in reciprocal

| # | affy id | HUGO name | Title | MapLocation |
|---|---|---|---|---|
| 1 | 203329_at | PTPRM | protein tyrosine phosphatase, receptor type, M | 18p11.2 |
| 2 | 219892_at | TM6SF1 | transmembrane 6 superfamily member 1 | 15q24-q26 |
| 3 | 205076_s_at | CRA | cisplatin resistance associated | 1q12-q21 |
| 4 | 204163_at | EMILIN | elastin microfibril interface located protein | 2p23.3-p23.2 |
| 5 | 224773_at | NAV1 | neuron navigator 1 | |
| 6 | 200660_at | S100A11 | S100 calcium binding protein A11 | 1q21 |
| | | | (calgizzarin) | |
| 7 | 210992_x_at | FCGR2A | Fc fragment of IgG, low affinity IIa, receptor | 1q23 |
| | | | for (CD32) | |
| 8 | 221879_at | MGC4809 | serologically defined breast cancer antigen | 15q22.2 |
| | | | NY-BR-20 | |
| 9 | 224774_s_at | NAV1 | neuron navigator 1 | |
| 10 | 201666_at | TIMP1 | tissue inhibitor of metalloproteinase 1 | Xp11.3-p11.23 |
| | | | (erythroid potentiating activity, collagenase | |
| | | | inhibitor) | |
| 11 | 218831_s_at | FCGRT | Fc fragment of IgG, receptor, transporter, | 19q13.3 |
| | | | alpha | |
| 12 | 205131_x_at | SCGF | stem cell growth factor; lymphocyte secreted | 19q13.3 |
| | | | C-type lectin | |
| 13 | 216236_s_at | SLC2A3 | solute carrier family 2 (facilitated glucose | 12p13.3 |
| | | | transporter), member 3 | |
| 14 | 206580_s_at | EFEMP2 | EGF-containing fibulin-like extracellular | 11q13 |
| | | | matrix protein 2 | |
| 15 | 208581_x_at | MT1X | metallothionein 1X | 16q13 |
| 16 | 210783_x_at | SCGF | stem cell growth factor; lymphocyte secreted | 19q13.3 |
| | | | C-type lectin | |

| | | | Sequence | |
|---|---|---|---|---|
| # | Sequence Type | Transcript ID | Derived From | Sequence ID |
| 1 | Exemplarsequence | Hs.154151.0 | NM_002845.1 | g4506318 |
| 2 | Exemplarsequence | Hs.133865.0 | NM_023003.1 | g13194198 |
| 3 | Exemplarsequence | Hs.166066.0 | NM_006697.1 | g5870890 |
| 4 | Exemplarsequence | Hs.63348.0 | NM_007046.1 | g5901943 |
| 5 | Consensussequence | Hs.6298.0 | AB032977.1 | Hs.6298.0 |
| 6 | Exemplarsequence | Hs.256290.0 | NM_005620.1 | g5032056 |
| 7 | Exemplarsequence | Hs.78864.1 | U90939.1 | g2149627 |
| 8 | Consensussequence | Hs.239812.0 | AA886335 | Hs.239812.0.S1 |
| 9 | Consensussequence | Hs.6298.0 | AB032977.1 | Hs.6298.0 |
| 10 | Exemplarsequence | Hs.5831.0 | NM_003254.1 | g4507508 |
| 11 | Exemplarsequence | Hs.111903.0 | NM_004107.1 | g4758345 |
| 12 | Exemplarsequence | Hs.105927.0 | NM_002975.1 | g4506802 |
| 13 | Consensussequence | Hs.7594.2 | AL110298.1 | Hs.7594.2.A1 |
| 14 | Exemplarsequence | Hs.6059.0 | NM_016938.1 | g8393298 |
| 15 | Exemplarsequence | Hs.278462.0 | NM_005952.1 | g10835231 |
| 16 | Exemplarsequence | Hs.105927.1 | D86586.1 | g2257694 |

| | Sequence | | | | |
|---|---|---|---|---|---|
| # | Source | Unigene_Accession | Cluster_Type | LocusLink | Full_Length_Reference_Seq |
| 1 | RefSeq | Hs.154151 | fulllength | 5797 | NM_002845; protein tyrosine phosphatase, receptor |
| | | | | | type, M precursor |
| 2 | RefSeq | Hs.341203 | fulllength | 53346 | NM_023003; transmembrane 6 superfamily member 1 |
| 3 | RefSeq | Hs.166066 | fulllength | 10903 | NM_006697; cisplatin resistance associated |
| 4 | RefSeq | Hs.63348 | fulllength | 11117 | NM_007046; elastin microfibril interface located protein |
| 5 | GenBank | Hs.6298 | fulllength | 89796 | NM_020443; neuron navigator 1 |
| 6 | RefSeq | Hs.417004 | fulllength | 6282 | NM_005620; S100 calcium binding protein A11 |
| | | | | | (calgizzarin) |
| 7 | GenBank | Hs.78864 | fulllength | 2212 | NM_021642; Fc fragment of IgG, low affinity IIa, receptor |
| | | | | | for (CD32) |
| 8 | GenBank | Hs.250861 | fulllength | 91860 | |
| 9 | GenBank | Hs.6298 | fulllength | 89796 | NM_020443; neuron navigator 1 |
| 10 | RefSeq | Hs.5831 | fulllength | 7076 | NM_003254; tissue inhibitor of metalloproteinase 1 |
| | | | | | precursor |
| 11 | RefSeq | Hs.111903 | fulllength | 2217 | NM_004107; Fc fragment of IgG, receptor, transporter, |
| | | | | | alpha |
| 12 | RefSeq | Hs.105927 | fulllength | 6320 | NM_002975; stem cell growth factor; lymphocyte |
| | | | | | secreted C-type lectin |
| 13 | GenBank | Hs.7594 | fulllength | 6515 | NM_006931; solute carrier family 2 (facilitated glucose |
| | | | | | transporter), member 3 NM_153449; glucose transporter |
| | | | | | 14 |
| 14 | RefSeq | Hs.6059 | fulllength | 30008 | NM_016938; EGF-containing fibulin-like extracellular |
| | | | | | matrix protein 2 |
| 15 | RefSeq | Hs.374950 | fulllength | 4501 | NM_005952; metallothionein 1X |
| 16 | GenBank | Hs.105927 | fulllength | 6320 | NM_002975; stem cell growth factor; lymphocyte |
| | | | | | secreted C-type lectin |

TABLE 3
genes lower expressed in CEBPA than in reciprocal

| # | affy id | HUGO name | Title | MapLocation |
|---|---|---|---|---|
| 1 | 206761_at | TACTILE | T cell activation, increased late expression | 3q13.13 |
| 2 | 232424_at | PRDM16 | PR domain containing 16 | 1p36.23-p33 |
| 3 | 219054_at | FLJ14054 | hypothetical protein FLJ14054 | 5p13.3 |
| 4 | 202746_at | ITM2A | integral membrane protein 2A | Xq13.3-Xq21.2 |
| 5 | 202747_s_at | ITM2A | integral membrane protein 2A | Xq13.3-Xq21.2 |
| 6 | 210665_at | TFPI | tissue factor pathway inhibitor (lipoprotein- | 2q31-q32.1 |
| | | | associated coagulation inhibitor) | |
| 7 | 226751_at | DKFZP566K1924 | DKFZP566K1924 protein | 2p13.2 |
| 8 | 219790_s_at | NPR3 | natriuretic peptide receptor C/guanylate | 5p14-p13 |
| | | | cyclase C (atrionatriuretic peptide receptor C) | |
| 9 | 219837_s_at | C17 | cytokine-like protein C17 | 4p16-p15 |
| 10 | 206660_at | IGLL1 | immunoglobulin lambda-like polypeptide 1 | 22q11.23 |
| 11 | 210762_s_at | DLC1 | deleted in liver cancer 1 | 8p22-p21.3 |
| 12 | 209757_s_at | MYCN | v-myc myelocytomatosis viral related | 2p24.1 |
| | | | oncogene, neuroblastoma derived (avian) | |
| 13 | 219789_at | NPR3 | natriuretic peptide receptor C/guanylate | 5p14-p13 |
| | | | cyclase C (atrionatriuretic peptide receptor C) | |
| 15 | 226517_at | BCAT1 | branched chain aminotransferase 1, cytosolic | 12pter-q12 |
| 16 | 210664_s_at | TFPI | tissue factor pathway inhibitor (lipoprotein- | 2q31-q32.1 |
| | | | associated coagulation inhibitor) | |
| 17 | 219686_at | HSA250839 | gene for serine/threonine protein kinase | 4p16.2 |
| 18 | 209160_at | AKR1C3 | aldo-keto reductase family 1, member C3 (3- | 10p15-p14 |
| | | | alpha hydroxysteroid dehydrogenase, type II) | |

| | | | Sequence | |
|---|---|---|---|---|
| # | Sequence Type | Transcript ID | Derived From | Sequence ID |
| 1 | Exemplarsequence | Hs.142023.0 | NM_005816.1 | g5032140 |
| 2 | Consensussequence | Hs.302022.1 | AI623202 | Hs.302022.1.S1 |
| 3 | Exemplarsequence | Hs.13528.0 | NM_024563.1 | g13375730 |
| 4 | Consensussequence | Hs.17109.0 | AL021786 | Hs.17109.0_RC |
| 5 | Exemplarsequence | Hs.17109.0 | NM_004867.1 | g4758223 |
| 6 | Exemplarsequence | Hs.170279.1 | AF021834.1 | g4103170 |
| 7 | Consensussequence | Hs.26358.0 | AW193693 | Hs.26358.0.S1 |
| 8 | Exemplarsequence | Hs.123655.0 | NM_000908.1 | g4505440 |
| 9 | Exemplarsequence | Hs.13872.0 | NM_018659.1 | g8922107 |
| 10 | Exemplarsequence | Hs.288168.0 | NM_020070.1 | g13399297 |
| 11 | Exemplarsequence | Hs.8700.0 | AF026219.1 | g2559001 |
| 12 | Exemplarsequence | Hs.25960.1 | BC002712.1 | g12803748 |
| 13 | Consensussequence | Hs.123655.0 | AI628360 | Hs.123655.0 |
| 15 | Consensussequence | Hs.317432.0 | AL390172.1 | Hs.317432.0.S1 |
| 16 | Exemplarsequence | Hs.170279.1 | AF021834.1 | g4103170 |
| 17 | Exemplarsequence | Hs.58241.0 | NM_18401.1 | g8923753 |
| 18 | Exemplarsequence | Hs.78183.0 | AB018580.1 | g6624210 |

| | Sequence | | | | |
|---|---|---|---|---|---|
| # | Source | Unigene_Accession | Cluster_Type | LocusLink | Full_Length_Reference_Seq |
| 1 | RefSeq | Hs.142023 | fulllength | 10225 | NM_005816; T cell activation, increased late expression |
| 2 | GenBank | Hs.302022 | fulllength | 63976 | NM_022114; PR domain containing 16 |
| 3 | RefSeq | Hs.13528 | fulllength | 79614 | NM_024563; hypothetical protein FLJ14054 |
| 4 | GenBank | Hs.17109 | fulllength | 9452 | NM_004867; integral membrane protein 2A |
| 5 | RefSeq | Hs.17109 | fulllength | 9452 | NM_004867; integral membrane protein 2A |
| 6 | GenBank | Hs.170279 | fulllength | 7035 | NM_006287; tissue factor pathway inhibitor (lipoprotein-associated |
| | | | | | coagulation inhibitor) |
| 7 | GenBank | Hs.26358 | fulllength | 25927 | NM_015463; DKFZP566K1924 protein |
| 8 | RefSeq | Hs.123655 | fulllength | 4883 | NM_000908; natriuretic peptide receptor C/guanylate cyclase C |
| | | | | | (atrionatriuretic peptide receptor C) |
| 9 | RefSeq | Hs.13872 | fulllength | 54360 | NM_018659; cytokine-like protein C17 |
| 10 | RefSeq | Hs.348935 | fulllength | 3543 | NM_020070; immunoglobulin lambda-like polypeptide 1 isoform a |
| | | | | | precursor NM_152855; immunoglobulin lambda-like polypeptide 1 |
| | | | | | isoform b precursor |
| 11 | GenBank | Hs.8700 | fulllength | 10395 | NM_006094; deleted in liver cancer 1 NM_024767; deleted in liver |
| | | | | | cancer 1 |
| 12 | GenBank | Hs.25960 | fulllength | 4613 | NM_005378; v-myc myelocytomatosis viral related oncogene, |
| | | | | | neuroblastoma derived |
| 13 | GenBank | Hs.123655 | fulllength | 4883 | NM_000908; natriuretic peptide receptor C/guanylate cyclase C |
| | | | | | (atrionatriuretic peptide receptor C) |
| 15 | GenBank | Hs.317432 | fulllength | 586 | NM_005504; branched chain aminotransferase 1, cytosolic |
| 16 | GenBank | Hs.170279 | fulllength | 7035 | NM_006287; tissue factor pathway inhibitor (lipoprotein-associated |
| | | | | | coagulation inhibitor) |
| 17 | RefSeq | Hs.58241 | fulllength | 55351 | NM_018401; gene for serine/threonine protein kinase |
| 18 | GenBank | Hs.78183 | fulllength | 8644 | NM_003739; aldo-keto reductase family 1, member C3 |

TABLE 4
genes lower expressed in CEBPA than in t(11q23)

| | | | | | | Transcript | Sequence | |
|---|---|---|---|---|---|---|---|---|
| # | affy id | HUGO name | Title | MapLocation | Sequence Type | ID | Derived From | Sequence ID |
| 1 | 205472_s_at | DACH | dachshund homolog | 13q22 | Exemplarsequence | Hs.63931.0 | NM_004392.1 | g4758113 |
| | | | (Drosophila) | | | | | |
| 2 | 205471_s_at | DACH | dachshund homolog | 13q22 | Consensussequence | Hs.63931.0 | AW772082 | Hs.63931.0 |
| | | | (Drosophila) | | | | | |
| 3 | 225185_at | MRAS | muscle RAS oncogene | 3q22.3 | Consensussequence | Hs.7298.1 | BF343625 | Hs.7298.1_RC |
| | | | homolog | | | | | |
| 4 | 219360_s_at | TRPM4 | transient receptor potential | 19q13.33 | Exemplarsequence | Hs.31608.0 | NM_017636.1 | g8923048 |
| | | | cation channel, subfamily | | | | | |
| | | | M, member 4 | | | | | |
| 5 | 203372_s_at | SOCS2 | suppressor of cytokine | 12q | Exemplarsequence | Hs.110776.0 | AB004903.1 | g2443360 |
| | | | signaling 2 | | | | | |
| 6 | 203373_at | SOCS2 | suppressor of cytokine | 12q | Exemplarsequence | Hs.110776.0 | NM_003877.1 | g4507262 |
| | | | signaling 2 | | | | | |
| 7 | 228083_at | CACNA2D4 | calcium channel, | 12p13.33 | Consensussequence | Hs.13768.0 | AI433691 | Hs.13768.0 |
| | | | voltage-dependent, alpha 2/ | | | | | |
| | | | delta subunit 4 | | | | | |
| 8 | 219506_at | FLJ23221 | hypothetical protein | 1q21.2 | Exemplarsequence | Hs.18397.0 | NM_024579.1 | g13375757 |
| | | | FLJ23221 | | | | | |
| 9 | 200782_at | ANXA5 | annexin A5 | 4q28-q32 | Exemplarsequence | Hs.300711.0 | NM_001154.2 | g4809273 |
| 10 | 202265_at | BMI1 | B lymphoma Mo-MLV | 10p11.23 | Exemplarsequence | Hs.431.0 | NM_005180.1 | g4885094 |
| | | | insertion region (mouse) | | | | | |
| 11 | 218376_s_at | MICAL | CasL interacting molecule | 6q21 | Exemplarsequence | Hs.33476.0 | NM_022765.1 | g12232438 |
| 12 | 216041_x_at | GRN | granulin | 17q21.32 | Consensussequence | Hs.180577.2 | AK023348.1 | Hs.180577.2.S1 |

| | Sequence | | | | |
|---|---|---|---|---|---|
| # | Source | Unigene_Accession | Cluster_Type | LocusLink | Full_Length_Reference_Seq |
| 1 | RefSeq | Hs.63931 | fulllength | 1602 | NM_004392; dachshund homolog isoform c NM_080759; dachshund homolog |
| | | | | | isoform a NM_080760; dachshund homolog isoform b |
| 2 | GenBank | Hs.63931 | fulllength | 1602 | NM_004392; dachshund homolog isoform c NM_080759; dachshund homolog |
| | | | | | isoform a NM_080760; dachshund homolog isoform b |
| 3 | GenBank | Hs.349227 | fulllength | 22808 | NM_012219; muscle RAS oncogene homolog |
| 4 | RefSeq | Hs.31608 | fulllength | 54795 | NM_017636; transient receptor potential cation channel, subfamily M, member 4 |
| 5 | GenBank | Hs.405946 | fulllength | 8835 | NM_003877; suppressor of cytokine signaling-2 |
| 6 | RefSeq | Hs.405946 | fulllength | 8835 | NM_003877; suppressor of cytokine signaling-2 |
| 7 | GenBank | Hs.13768 | fulllength | 93589 | NM_172364; voltage-gated calcium channel alpha(2)delta-4 subunit |
| 8 | RefSeq | Hs.18397 | fulllength | 79630 | NM_024579; hypothetical protein FLJ23221 |
| 9 | RefSeq | Hs.300711 | fulllength | 308 | NM_001154; annexin 5 |
| 10 | RefSeq | Hs.380403 | fulllength | 648 | NM_005180; B lymphoma Mo-MLV insertion region |
| 11 | RefSeq | Hs.33476 | fulllength | 64780 | NM_022765; NEDD9 interacting protein with calponin homology |
| | | | | | and LIM domains |
| 12 | GenBank | Hs.180577 | fulllength | 2896 | NM_002087; granulin |

TABLE 5
genes higher expressed in CEBPA than in inv(16)

| | | HUGO | | | | Transcript | Sequence | |
|---|---|---|---|---|---|---|---|---|
| # | affy id | name | Title | MapLocation | Sequence Type | ID | Derived From | Sequence ID |
| 1 | 235438_at | | ESTs | | Consensussequence | Hs.146226.0 | AW162011 | Hs.146226.0_RC |
| 2 | 209905_at | HOXA9 | homeo box A9 | 7p15-p14 | Consensussequence | Hs.127428.0 | AI246769 | Hs.127428.0 |
| 3 | 235521_at | HOXA3 | homeo box A3 | 7p15-p14 | Consensussequence | Hs.222446.0 | AW137982 | Hs.222446.0.A1 |
| 4 | 214651_s_at | HOXA9 | homeo box A9 | 7p15-p14 | Consensussequence | Hs.127428.2 | U41813.1 | Hs.127428.2 |
| 5 | 211031_s_at | CYLN2 | cytoplasmic linker 2 | 7q11.23 | Exemplarsequence | g13623312 | BC006259.1 | g13623312 |
| 6 | 223044_at | SLC11A3 | solute carrier family 11 | 2q32 | Exemplarsequence | Hs.5944.0 | AL136944.1 | g12053382 |
| | | | (proton-coupled divalent | | | | | |
| | | | metal ion transporters), | | | | | |
| | | | member 3 | | | | | |
| 7 | 230894_s_at | | Homo sapiens, clone | | Consensussequence | Hs.42640.1 | BE672557 | Hs.42640.1.A1 |
| | | | IMAGE: 4154313, mRNA, | | | | | |
| | | | partial cds | | | | | |
| 8 | 200985_s_at | CD59 | CD59 antigen p18-20 | 11p13 | Exemplarsequence | Hs.119663.0 | NM_000611.1 | g10835164 |
| | | | (antigen identified by | | | | | |
| | | | monoclonal antibodies | | | | | |
| | | | 16.3A5, EJ16, | | | | | |
| | | | EJ30, EL32 and G344) | | | | | |
| 9 | 218927_s_at | C4S-2 | chondroitin 4-O- | 7p22 | Exemplarsequence | Hs.25204.0 | NM_018641.1 | g8922111 |
| | | | sulfotransferase 2 | | | | | |
| 10 | 201427_s_at | SEPP1 | selenoprotein P, plasma, 1 | 5q31 | Exemplarsequence | Hs.3314.0 | NM_005410.1 | g4885590 |
| 11 | 212463_at | | Homo sapiens mRNA; | | Consensussequence | Hs.99766.0 | BE379006 | Hs.99766.0.S1 |
| | | | cDNA DKFZp564J0323 | | | | | |
| | | | (from clone | | | | | |
| | | | DKFZp564J0323) | | | | | |
| 12 | 201669_s_at | MARCKS | myristoylated alanine-rich | 6q22.2 | Exemplarsequence | Hs.75607.0 | NM_002356.4 | g11125771 |
| | | | protein kinase C substrate | | | | | |
| 13 | 219218_at | FLJ23058 | hypothetical protein | 17q25.3 | Exemplarsequence | Hs.98968.0 | NM_024696.1 | g13375978 |
| | | | FLJ23058 | | | | | |
| 14 | 201670_s_at | MARCKS | myristoylated alanine-rich | 6q22.2 | Exemplarsequence | Hs.75607.0 | M68956.1 | g187386 |
| | | | protein kinase C substrate | | | | | |
| 15 | 200983_x_at | CD59 | CD59 antigen p18-20 | 11p13 | Consensussequence | Hs.119663.0 | NM_000611.1 | Hs.119663.0 |
| | | | (antigen | | | | | |
| | | | identified by monoclonal | | | | | |
| | | | antibodies 16.3A5, EJ16, | | | | | |
| | | | EJ30, EL32 and G344) | | | | | |
| 16 | 210215_at | TFR2 | transferrin receptor 2 | 7q22 | Exemplarsequence | Hs.63758.1 | AF067864.1 | g5596369 |
| 17 | 235753_at | | Homo sapiens cDNA | | Consensussequence | Hs.196169.0 | AI492051 | Hs.196169.0 |
| | | | FLJ34835 fis, clone | | | | | |
| | | | NT2NE2010150. | | | | | |
| 18 | 204720_s_at | DNAJC6 | DnaJ (Hsp40) homolog, | 1pter-q31.3 | Consensussequence | Hs.44896.0 | AV729634 | Hs.44896.0 |
| | | | subfamily C, member 6 | | | | | |
| 19 | 212224_at | ALDH1A1 | aldehyde dehydrogenase 1 | 9q21.13 | Consensussequence | Hs.76392.0 | NM_000689.1 | Hs.76392.0 |
| | | | family, member A1 | | | | | |
| 20 | 243579_at | MSI2 | musashi homolog 2 | 17q23.1 | Consensussequence | Hs.173179.0 | BF029215 | Hs.173179.0.S1 |
| | | | (Drosophila) | | | | | |
| 21 | 205830_at | CLGN | calmegin | 4q28.3-q31.1 | Exemplarsequence | Hs.86368.0 | NM_004362.1 | g4758003 |
| 22 | 210425_x_at | GOLGIN- | golgin-67 | 15q11.2 | Exemplarsequence | Hs.182982.1 | AF164622.1 | g7211437 |
| | | 67 | | | | | | |
| 23 | 209691_s_at | DOK4 | docking protein 4 | 16q12.2 | Exemplarsequence | Hs.279832.1 | BC003541.1 | g13097653 |

| | Sequence | | | | |
|---|---|---|---|---|---|
| # | Source | Unigene_Accession | Cluster_Type | LocusLink | Full_Length_Reference_Seq |
| 1 | GenBank | Hs.445509 | est | | |
| 2 | GenBank | Hs.127428 | fulllength | 3205 | NM_002142; homeobox protein A9 isoform b |
| | | | | | NM_152739; homeobox protein A9 isoform a |
| 3 | GenBank | Hs.248074 | fulllength | 3200 | NM_030661; homeobox A3 protein isoform a |
| | | | | | NM_153631; homeobox A3 protein isoform a |
| | | | | | NM_153632; homeobox A3 protein isoform b |
| 4 | GenBank | Hs.127428 | fulllength | 3205 | NM_002142; homeobox protein A9 isoform b |
| | | | | | NM_152739; homeobox protein A9 isoform a |
| 5 | GenBank | Hs.104717 | fulllength | 7461 | NM_003388; cytoplasmic linker 2 isoform 1 |
| | | | | | NM_032421; cytoplasmic linker 2 isoform 2 |
| | | | | | NM_032719; |
| 6 | GenBank | Hs.5944 | fulllength | 30061 | NM_014585; solute carrier family 40 (iron-regulated |
| | | | | | transporter), member 1 |
| 7 | GenBank | Hs.173179 | | | |
| 8 | RefSeq | Hs.278573 | fulllength | 966 | NM_000611; CD59 antigen p18-20 (antigen identified |
| | | | | | by monoclonal antibodies 16.3A5, EJ16, EJ30, EL32 |
| | | | | | and G344) |
| 9 | RefSeq | Hs.25204 | fulllength | 55501 | NM_018641; chondroitin 4-O-sulfotransferase 2 |
| 10 | RefSeq | Hs.275775 | fulllength | 6414 | NM_005410; selenoprotein P precursor |
| 11 | GenBank | Hs.99766 | | | |
| 12 | RefSeq | Hs.75607 | fulllength | 4082 | NM_002356; myristoylated alanine-rich protein kinase C |
| | | | | | substrate |
| 13 | RefSeq | Hs.98968 | fulllength | 79749 | NM_024696; hypothetical protein FLJ23058 |
| 14 | GenBank | Hs.75607 | fulllength | 4082 | NM_002356; myristoylated alanine-rich protein kinase C |
| | | | | | substrate |
| 15 | GenBank | Hs.278573 | fulllength | 966 | NM_000611; CD59 antigen p18-20 (antigen identified |
| | | | | | by monoclonal antibodies 16.3A5, EJ16, EJ30, EL32 |
| | | | | | and G344) |
| 16 | GenBank | Hs.63758 | fulllength | 7036 | NM_003227; transferrin receptor 2 |
| 17 | GenBank | Hs.196169 | | | |
| 18 | GenBank | Hs.44896 | fulllength | 9829 | NM_014787; DnaJ (Hsp40) homolog, subfamily C, |
| | | | | | member 6 |
| 19 | GenBank | Hs.76392 | fulllength | 216 | NM_000689; aldehyde dehydrogenase 1A1 |
| 20 | GenBank | Hs.103512 | fulllength | 124540 | NM_138962; musashi 2 isoform a NM_170721; |
| | | | | | musashi 2 isoform b |
| 21 | RefSeq | Hs.86368 | fulllength | 1047 | NM_004362; calmegin |
| 22 | GenBank | Hs.182982 | fulllength | 23015 | NM_015003; golgin-67 isoform a NM_181076; golgin- |
| | | | | | 67 isoform b NM_181077; golgin-67 isoform c |
| 23 | GenBank | Hs.279832 | fulllength | 55715 | NM_018110; downstream of tyrosine kinase 4 |

TABLE 6
genes lower expressed in CEBPA than in inv(16)

| | | HUGO | | |
|---|---|---|---|---|
| | affy id | name | Title | MapLocation |
| 1 | 204885_s_at | MSLN | mesothelin | 16p13.3 |
| 2 | 201497_x_at | MYH11 | myosin, heavy polypeptide 11, smooth muscle | 16p13.13-p13.12 |
| 3 | 205819_at | MARCO | macrophage receptor with collagenous structure | 2q12-q13 |
| 4 | 207961_x_at | MYH11 | myosin, heavy polypeptide 11, smooth muscle | 16p13.13-p13.12 |
| 5 | 206135_at | ST18 | suppression of tumorigenicity 18 (breast carcinoma) | 8q11.22 |
| | | | (zinc finger protein) | |
| 6 | 241525_at | LOC200772 | hypothetical protein LOC200772 | 2q37.3 |
| 7 | 212358_at | CLIPR-59 | CLIP-170-related protein | 19q13.12 |
| 8 | 230472_at | IRX1 | iroquois homeobox protein 1 | 5p15.3 |
| 9 | 222760_at | FLJ14299 | hypothetical protein FLJ14299 | 8p11.22 |
| 10 | 208450_at | LGALS2 | lectin, galactoside-binding, soluble, 2 (galectin 2) | 22q13.1 |
| 11 | 201506_at | TGFBI | transforming growth factor, beta-induced, 68 kDa | 5q31 |
| 12 | 222862_s_at | AK5 | adenylate kinase 5 | 1p31 |
| 13 | 201743_at | CD14 | CD14 antigen | 5q31.1 |
| 14 | 204163_at | EMILIN | elastin microfibril interface located protein | 2p23.3-p23.2 |
| 15 | 206682_at | HML2 | macrophage lectin 2 (calcium dependent) | 17p13.2 |
| 16 | 218876_at | CGI-38 | brain specific protein | 16q21 |
| 17 | 203939_at | NT5E | 5′-nucleotidase, ecto (CD73) | 6q14.q21 |
| 18 | 203407_at | PPL | periplakin | 16p13.3 |
| 19 | 224724_at | SULF2 | similar to glucosamine-6-sulfatases | 20q12-13.2 |
| 20 | 238066_at | CRBPIV | retinoid binding protein 7 | 1p36.22 |

| | | | Sequence | |
|---|---|---|---|---|
| | Sequence Type | Transcript ID | Derived From | Sequence ID |
| 1 | Exemplarsequence | Hs.155981.0 | NM_005823.2 | g7108357 |
| 2 | Exemplarsequence | Hs.78344.1 | NM_022844.1 | g13124874 |
| 3 | Exemplarsequence | Hs.67726.0 | NM_006770.1 | g5803079 |
| 4 | Exemplarsequence | Hs.78344.2 | NM_022870.1 | g13124876 |
| 5 | Exemplarsequence | Hs.151449.0 | NM_014682.1 | g7662167 |
| 6 | Consensussequence | Hs.132051.0 | AV700191 | Hs.132051.0.A1 |
| 7 | Consensussequence | Hs.7357.0 | AL117468.1 | Hs.7357.0.S1 |
| 8 | Consensussequence | Hs.109525.0 | AI870306 | Hs.109525.0.A1 |
| 9 | Consensussequence | Hs.288042.0 | BG290193 | Hs.288042.0_RC |
| 10 | Exemplarsequence | Hs.113987.0 | NM_006498.1 | g5729902 |
| 11 | Exemplarsequence | Hs.118787.0 | NM_000358.1 | g4507466 |
| 12 | Consensussequence | Hs.18268.0 | BG169832 | Hs.18268.0 |
| 13 | Exemplarsequence | Hs.75627.0 | NM_000591.1 | g4557416 |
| 14 | Exemplarsequence | Hs.63348.0 | NM_007046.1 | g5901943 |
| 15 | Exemplarsequence | Hs.54403.0 | NM_006344.1 | g5453683 |
| 16 | Exemplarsequence | Hs.279772.0 | NM_016140.1 | g7706392 |
| 17 | Exemplarsequence | Hs.153952.0 | NM_002526.1 | g4505466 |
| 18 | Exemplarsequence | Hs.74304.0 | NM_002705.1 | g4505992 |
| 19 | Consensussequence | Hs.43857.0 | AL133001.1 | Hs.43857.0.S1 |
| 20 | Consensussequence | Hs.292718.0 | AI733027 | Hs.292718.0_RC |

| | Sequence | | | | |
|---|---|---|---|---|---|
| | Source | Unigene_Accession | Cluster_Type | LocusLink | Full_Length_Reference_Seq |
| 1 | RefSeq | Hs.155981 | fulllength | 10232 | NM_005823; megakaryocyte potentiating factor precursor |
| | | | | | NM_013404; mesothelin isoform 2 precursor |
| 2 | RefSeq | Hs.78344 | fulllength | 4629 | NM_002474; smooth muscle myosin heavy chain 11 isoform SM1 |
| | | | | | NM_022844; smooth muscle myosin heavy chain 11 isoform SM2 |
| | | | | | NM_022870; |
| 3 | RefSeq | Hs.67726 | fulllength | 8685 | NM_006770; macrophage receptor with collagenous structure |
| 4 | RefSeq | Hs.78344 | fulllength | 4629 | NM_002474; smooth muscle myosin heavy chain 11 isoform SM1 |
| | | | | | NM_022844; smooth muscle myosin heavy chain 11 isoform SM2 |
| | | | | | NM_022870; |
| 5 | RefSeq | Hs.151449 | fulllength | 9705 | NM_014682; suppression of tumorigenicity 18 |
| 6 | GenBank | Hs.439538 | | 200772 | |
| 7 | GenBank | Hs.7357 | fulllength | 25999 | NM_015526; CLIP-170-related protein |
| 8 | GenBank | Hs.424156 | | 79192 | |
| 9 | GenBank | Hs.288042 | fulllength | 80139 | NM_025069; hypothetical protein FLJ14299 |
| 10 | RefSeq | Hs.113987 | fulllength | 3957 | NM_006498; lectin, galactoside-binding, soluble, 2 (galectin 2) |
| 11 | RefSeq | Hs.118787 | fulllength | 7045 | NM_000358; transforming growth factor, beta-induced, 68 kDa |
| 12 | GenBank | Hs.18268 | fulllength | 26289 | NM_012093; adenylate kinase 5 isoform 2 NM_174858; |
| | | | | | adenylate kinase 5 isoform 1 |
| 13 | RefSeq | Hs.75627 | fulllength | 929 | NM_000591; CD14 antigen precursor |
| 14 | RefSeq | Hs.63348 | fulllength | 11117 | NM_007046; elastin microfibril interface located protein |
| 15 | RefSeq | Hs.54403 | fulllength | 10462 | NM_006344; macrophage lectin 2 (calcium dependent) |
| 16 | RefSeq | Hs.279772 | fulllength | 51673 | NM_015964; CGI-38 protein NM_016140; brain specific protein |
| 17 | RefSeq | Hs.153952 | fulllength | 4907 | NM_002526; 5′ nucleotidase, ecto |
| 18 | RefSeq | Hs.74304 | fulllength | 5493 | NM_002705; periplakin |
| 19 | GenBank | Hs.43857 | fulllength | 55959 | NM_018837; similar to glucosamine-6-sulfatases |
| 20 | GenBank | Hs.422688 | fulllength | 116362 | NM_052960; retinoid binding protein 7 |

TABLE 7
genes higher expressed in CEBPA than in inv(3)

| | | | | | | | Sequence |
|---|---|---|---|---|---|---|---|
| # | affy id | HUGO name | Title | MapLocation | Sequence Type | Transcript ID | Derived From |
| 1 | 204561_x_at | APOC2 | apolipoprotein C-II | 19q13.2 | Exemplarsequence | Hs.75615.0 | NM_000483.2 |
| 2 | 210997_at | HGF | hepatocyte growth factor (hepapoietin A; | 7q21.1 | Exemplarsequence | Hs.809.1 | M77227.1 |
| | | | scatter factor) | | | | |
| 3 | 213110_s_at | COL4A5 | collagen, type IV, alpha 5 (Alport syndrome) | Xq22 | Consensussequence | Hs.169825.0 | AW052179 |
| 4 | 206622_at | TRH | thyrotropin-releasing hormone | 3q13.3-q21 | Exemplarsequence | Hs.182231.0 | NM_007117.1 |
| 5 | 210549_s_at | CCL23 | chemokine (C-C motif) ligand 23 | 17q12 | Exemplarsequence | Hs.169191.1 | U58913.1 |
| 6 | 210998_s_at | HGF | hepatocyte growth factor (hepapoietin A; | 7q21.1 | Exemplarsequence | Hs.809.1 | M77227.1 |
| | | | scatter factor) | | | | |
| 7 | 236892_s_at | | Homo sapiens, clone MGC: 10077 | | Consensussequence | Hs.269918.0 | BF590528 |
| | | | IMAGE: 3896690, mRNA, | | | | |
| | | | complete cds | | | | |
| 8 | 239791_at | | Homo sapiens, clone MGC: 10077 | | Consensussequence | Hs.269918.1 | AI125255 |
| | | | IMAGE: 3896690, mRNA, complete cds | | | | |
| 9 | 232424_at | PRDM16 | PR domain containing 16 | 1p36.23-p33 | Consensussequence | Hs.302022.1 | AI623202 |
| 10 | 206210_s_at | CETP | cholesteryl ester transfer protein, plasma | 16q21 | Exemplarsequence | Hs.89538.0 | NM_000078.1 |
| 11 | 205624_at | CPA3 | carboxypeptidase A3 (mast cell) | 3q21-q25 | Exemplarsequence | Hs.646.0 | NM_001870.1 |
| 12 | 228293_at | LOC91614 | novel 58.3 KDA protein | 11p13 | Consensussequence | Hs.180545.0 | AJ245600.1 |
| 13 | 206660_at | IGLL1 | immunoglobulin lambda-like polypeptide 1 | 22q11.23 | Exemplarsequence | Hs.288168.0 | NM_020070.1 |
| 14 | 213844_at | HOXA5 | homeo box A5 | 7p15-p14 | Consensussequence | Hs.37034.0 | NM_019102.1 |
| 15 | 209960_at | HGF | hepatocyte growth factor (hepapoietin A; | 7q21.1 | Consensussequence | Hs.809.0 | X16323.1 |
| | | | scatter factor) | | | | |
| 16 | 210762_s_at | DLC1 | deleted in liver cancer 1 | 8p22-p21.3 | Exemplarsequence | Hs.8700.0 | AF026219.1 |
| 17 | 228904_at | | ESTs | | Consensussequence | Hs.156044.0 | AW510657 |
| 18 | 204082_at | PBX3 | pre-B-cell leukemia transcription factor 3 | 9q33-q34 | Exemplarsequence | Hs.294101.0 | NM_006195.1 |

| | | Sequence | | | | |
|---|---|---|---|---|---|---|
| # | Sequence ID | Source | Unigene_Accession | Cluster_Type | LocusLink | Full_Length_Reference_Seq |
| 1 | g5174775 | RefSeq | Hs.75615 | fulllength | 344 | NM_000483; apolipoprotein C-II precursor |
| 2 | g184029 | GenBank | Hs.809 | fulllength | 3082 | NM_000601; hepatocyte growth factor precursor |
| 3 | Hs.169825.0_RC | GenBank | Hs.169825 | fulllength | 1287 | IV collagen isoform 2, precursor NM_033381; alpha |
| | | | | | | 5 type IV collagen isoform 3, precursor |
| 4 | g6005919 | RefSeq | Hs.182231 | fulllength | 7200 | NM_007117; thyrotropin-releasing hormone |
| 5 | g4204907 | GenBank | Hs.169191 | fulllength | 6368 | NM_005064; small inducible cytokine A23 isoform |
| | | | | | | CKbeta8-1 precursor NM_145898; |
| | | | | | | small inducible cytokine A23 isoform CKbeta8 precursor |
| 6 | g184029 | GenBank | Hs.809 | fulllength | 3082 | NM_000601; hepatocyte growth factor precursor |
| 7 | Hs.269918.0.A1 | GenBank | Hs.183096 | fulllength | | |
| 8 | Hs.269918.1.A1 | GenBank | Hs.183096 | fulllength | | |
| 9 | Hs.302022.1.S1 | GenBank | Hs.302022 | fulllength | 63976 | NM_022114; PR domain containing 16 |
| 10 | g4557442 | RefSeq | Hs.89538 | fulllength | 1071 | NM_000078; cholesteryl ester transfer protein, plasma precursor |
| 11 | g4503000 | RefSeq | Hs.646 | fulllength | 1359 | NM_001870; mast cell carboxypeptidase A3 precursor |
| 12 | Hs.180545.0 | GenBank | Hs.180545 | fulllength | 91614 | NM_139160; novel 58.3 KDA protein |
| 13 | g13399297 | RefSeq | Hs.348935 | fulllength | 3543 | NM_020070; immunoglobulin lambda-like |
| | | | | | | polypeptide 1 isoform a precursor |
| | | | | | | NM_152855; immunoglobulin lambda-like polypeptide |
| | | | | | | 1 isoform b precursor |
| 14 | Hs.37034.0.S1 | GenBank | Hs.37034 | fulllength | 3202 | NM_019102; homeobox protein A5 |
| 15 | Hs.809.0 | GenBank | Hs.809 | fulllength | 3082 | NM_000601; hepatocyte growth factor precursor |
| 16 | g2559001 | GenBank | Hs.8700 | fulllength | 10395 | NM_006094; deleted in liver cancer 1 NM_024767; |
| | | | | | | deleted in liver cancer 1 |
| 17 | Hs.156044.0 | GenBank | Hs.156044 | est | | |
| 18 | g5453851 | RefSeq | Hs.294101 | fulllength | 5090 | NM_006195; pre-B-cell leukemia transcription factor 3 |

TABLE 8
genes lower expressed in CEBPA than in inv(3)

| | | HUGO | | | | Transcript | Sequence |
|---|---|---|---|---|---|---|---|
| # | affy id | name | Title | MapLocation | Sequence Type | ID | Derived From |
| 1 | 221884_at | EvI1 | ecotropic viral integration site 1 | 3q24-q28 | Consensussequence | Hs.234773.0 | BE466525 |
| 2 | 226420_at | EVI1 | ecotropic viral integration site 1 | 3q24-q28 | Consensussequence | Hs.234773.0 | AK025934.1 |
| 3 | 213201_s_at | TNNT1 | troponin T1, skeletal, slow | 19q13.4 | Consensussequence | Hs.73980.1 | AJ011712 |
| 4 | 202269_x_at | GBP1 | guanylate binding protein 1, | 1p22.2 | Exemplarsequence | Hs.62661.0 | BC002666.1 |
| | | | interferon-inducible, 67 kDa | | | | |
| 5 | 231577_s_at | GBP1 | guanylate binding protein 1, interferon- | 1p22.2 | Consensussequence | Hs.62661.1 | AW014593 |
| | | | inducible, 67 kDa | | | | |
| 6 | 209602_s_at | GATA3 | GATA binding protein 3 | 10p15 | Consensussequence | Hs.169946.0 | AI796169 |
| 7 | 226837_at | SPRED1 | sprouty-related, EVH1 domain containing 1 | 15q13.3 | Consensussequence | Hs.94133.0 | BE967019 |
| 8 | 208820_at | PTK2 | PTK2 protein tyrosine kinase 2 | 8q24-qter | Consensussequence | Hs.740.1 | AL037339 |
| 9 | 226231_at | PAWR | PRKC, apoptosis, WT1, regulator | 12q21 | Consensussequence | Hs.42683.0 | AI189509 |
| 10 | 201743_at | CD14 | CD14 antigen | 5q31.1 | Exemplarsequence | Hs.75627.0 | NM_000591.1 |
| 11 | 213994_s_at | SPON1 | spondin 1, (f-spondin) extracellular matrix | 11p15.2 | Consensussequence | Hs.5378.1 | AI885290 |
| | | | protein | | | | |
| 12 | 207826_s_at | ID3 | inhibitor of DNA binding 3, dominant | 1p36.13-p36.12 | Exemplarsequence | Hs.76884.0 | NM_002167.1 |
| | | | negative helix-loop-helix protein | | | | |
| 13 | 231947_at | FLJ21269 | hypothetical protein FLJ21269 | 6q25.1 | Consensussequence | Hs.18160.0 | AI242583 |
| 14 | 202270_at | GBP1 | guanylate binding protein 1, interferon- | 1p22.2 | Exemplarsequence | Hs.62661.0 | NM_002053.1 |
| | | | inducible, 67 kDa | | | | |
| 15 | 203329_at | PTPRM | protein tyrosine phosphatase, receptor type, M | 18p11.2 | Exemplarsequence | Hs.154151.0 | NM_002845.1 |
| 16 | 215446_s_at | LOX | lysyl oxidase | 5q23.2 | Consensussequence | Hs.102267.3 | L16895 |
| 17 | 225369_at | ESAM | similar to endothelial cell-selective | 11q24.2 | Consensussequence | Hs.173840.0 | AL573851 |
| | | | adhesion molecule | | | | |
| 18 | 204627_s_at | ITGB3 | integrin, beta 3 (platelet glycoprotein IIIa, | 17q21.32 | Exemplarsequence | Hs.87149.0 | M35999.1 |
| | | | antigen CD61) | | | | |

| | | Sequence | | | | |
|---|---|---|---|---|---|---|
| # | Sequence ID | Source | Unigene_Accession | Cluster_Type | LocusLink | Full_Length_Reference_Seq |
| 1 | Hs.234773.0.S1 | GenBank | Hs.234773 | | 2122 | NM_005241; ecotropic viral integration site 1 |
| 2 | Hs.234773.0 | GenBank | Hs.234773 | | 2122 | NM_005241; ecotropic viral integration site 1 |
| 3 | Hs.73980.1.S1 | GenBank | Hs.73980 | fulllength | 7138 | NM_003283; troponin T1, skeletal, slow |
| 4 | g12803662 | GenBank | Hs.62661 | fulllength | 2633 | NM_002053; guanylate binding protein 1, interferon- |
| | | | | | | inducible, 67 kD |
| 5 | Hs.62661.1.A1 | GenBank | Hs.62661 | fulllength | 2633 | NM_002053; guanylate binding protein 1, |
| | | | | | | interferon-inducible, 67 kD |
| 6 | Hs.169946.0.S3 | GenBank | Hs.169946 | fulllength | 2625 | NM_002051; GATA binding protein 3 NM_032742; |
| 7 | Hs.94133.0_RC | GenBank | Hs.302718 | fulllength | 161742 | NM_152594; sprouty-related protein with EVH-1 domain 1 |
| 8 | Hs.740.1.S2 | GenBank | Hs.740 | fulllength | 5747 | NM_005607; PTK2 protein tyrosine kinase 2 |
| | | | | | | isoform b NM_153831; PTK2 protein |
| | | | | | | tyrosine kinase 2 isoform a |
| 9 | Hs.42683.0.A1 | GenBank | Hs.372504 | fulllength | 5074 | NM_002583; apoptosis response protein |
| 10 | g4557416 | RefSeq | Hs.75627 | fulllength | 929 | NM_000591; CD14 antigen precursor |
| 11 | Hs.5378.1 | GenBank | Hs.5378 | fulllength | 10418 | NM_006108; spondin 1, (f-spondin) extracellular matrix protein |
| | | | | | | NM_032720; |
| 12 | g10835060 | RefSeq | Hs.76884 | fulllength | 3399 | NM_002167; inhibitor of DNA binding 3, dominant |
| | | | | | | negative helix-loop-helix protein |
| 13 | Hs.18160.0.S1 | GenBank | Hs.18160 | fulllength | 80177 | NM_025107; myc target in myeloid cells 1 |
| 14 | g4503938 | RefSeq | Hs.62661 | fulllength | 2633 | NM_002053; guanylate binding protein 1, |
| | | | | | | interferon-inducible, 67 kD |
| 15 | g4506318 | RefSeq | Hs.154151 | fulllength | 5797 | NM_002845; protein tyrosine phosphatase, |
| | | | | | | receptor type, M precursor |
| 16 | Hs.102267.3 | GenBank | Hs.102267 | fulllength | 4015 | NM_002317; lysyl oxidase preproprotein |
| 17 | Hs.173840.0.S1 | GenBank | Hs.173840 | fulllength | 90952 | NM_138961; similar to endothelial cell-selective |
| | | | | | | adhesion molecule |
| 18 | g183532 | GenBank | Hs.87149 | fulllength | 3690 | NM_000212; integrin beta chain, beta 3 precursor |

TABLE 9
genes higher expressed in CEBPA than in t(8 21)

| # | affy id | HUGO name | Title | MapLocation |
|---|---|---|---|---|
| 1 | 220377_at | C14orf110 | chromosome 14 open reading frame 110 | 14q32.33 |
| 2 | 209905_at | HOXA9 | homeo box A9 | 7p15-p14 |
| 3 | 206310_at | SPINK2 | serine protease inhibitor, Kazal type, 2 (acrosin- | 4q12 |
| | | | trypsin inhibitor) | |
| 4 | 214651_s_at | HOXA9 | homeo box A9 | 7p15-p14 |
| 5 | 229461_x_at | MGC46680 | hypothetical protein MGC46680 | 1p31.1 |
| 6 | 205366_s_at | HOXB6 | homeo box B6 | 17q21.3 |
| 7 | 213150_at | HOXA10 | homeo box A10 | 7p15-p14 |
| 8 | 217963_s_at | NGFRAP1 | nerve growth factor receptor (TNFRSF16) | Xq22.1 |
| | | | associated protein 1 | |
| 9 | 205453_at | HOXB2 | homeo box B2 | 17q21-q22 |
| 10 | 204030_s_at | SCHIP1 | schwannomin interacting protein 1 | 3q25.33 |
| 11 | 208146_s_at | CPVL | carboxypeptidase, vitellogenic-like | 7p15-p14 |
| 12 | 235521_at | HOXA3 | homeo box A3 | 7p15-p14 |
| 13 | 236892_s_at | | Homo sapiens, clone MGC: 10077 | |
| | | | IMAGE: 3896690, mRNA, complete cds | |
| 14 | 213110_s_at | COL4A5 | collagen, type IV, alpha 5 (Alport syndrome) | Xq22 |
| 15 | 204069_at | MEIS1 | Meis1, myeloid ecotropic viral integration site 1 | 2p14.p13 |
| | | | homolog (mouse) | |
| 16 | 232424_at | PRDM16 | PR domain containing 16 | 1p36.23-p33 |
| 17 | 239791_at | | Homo sapiens, clone MGC: 10077 | |
| | | | IMAGE: 3896690, mRNA, complete cds | |
| 18 | 235438_at | | ESTs | |
| 19 | 213844_at | HOXA5 | homeo box A5 | 7p15-p14 |
| 20 | 217520_x_at | LOC283683 | hypothetical protein LOC283683 | 15q11.2 |
| 21 | 230894_s_at | | Homo sapiens, clone IMAGE: 4154313, mRNA, | |
| | | | partial cds | |
| 22 | 229971_at | GPR114 | G protein-coupled receptor 114 | 16q12.2 |
| 23 | 214450_at | CTSW | cathepsin W (lymphopain) | 11q13.1 |
| 24 | 213147_at | HOXA10 | homeo box A10 | 7p15-p14 |
| 25 | 214049_x_at | CD7 | CD7 antigen (p41) | 17q25.2-q25.3 |
| 26 | 224595_at | CDW92 | CDw92 antigen | 9q31.2 |

| | | | Sequence | |
|---|---|---|---|---|
| # | Sequence Type | Transcript ID | Derived From | Sequence ID |
| 1 | Exemplarsequence | Hs.128155.0 | NM_014151.1 | g7661757 |
| 2 | Consensussequence | Hs.127428.0 | AI246769 | Hs.127428.0 |
| 3 | Exemplarsequence | Hs.98243.0 | NM_021114.1 | g10863910 |
| 4 | Consensussequence | Hs.127428.2 | U41813.1 | Hs.127428.2 |
| 5 | Consensussequence | Hs.296235.0 | AI123532 | Hs.296235.0_RC |
| 6 | Exemplarsequence | Hs.98428.0 | NM_018952.1 | g9506792 |
| 7 | Consensussequence | Hs.110637.0 | NM_018951.1 | Hs.110637.0_RC |
| 8 | Exemplarsequence | Hs.17775.0 | NM_014380.1 | g7657043 |
| 9 | Exemplarsequence | Hs.2733.0 | NM_002145.1 | g4504464 |
| 10 | Exemplarsequence | Hs.61490.0 | NM_014575.1 | g7657539 |
| 11 | Exemplarsequence | g13786124 | NM_031311.1 | g13786124 |
| 12 | Consensussequence | Hs.222446.0 | AW137982 | Hs.222446.0.A1 |
| 13 | Consensussequence | Hs.269918.0 | BF590528 | Hs.269918.0.A1 |
| 14 | Consensussequence | Hs.169825.0 | AW052179 | Hs.169825.0_RC |
| 15 | Exemplarsequence | Hs.170177.0 | NM_002398.1 | g4505150 |
| 16 | Consensussequence | Hs.302022.1 | AI623202 | Hs.302022.1.S1 |
| 17 | Consensussequence | Hs.269918.1 | AI125255 | Hs.269918.1 |
| 18 | Consensussequence | Hs.146226.0 | AW162011 | Hs.146226.0_RC |
| 19 | Consensussequence | Hs.37034.0 | NM_019102.1 | Hs.37034.0.S1 |
| 20 | Consensussequence | Hs.154999.0 | BG396614 | Hs.154999.0.A1 |
| 21 | Consensussequence | Hs.42640.1 | BE672557 | Hs.42640.1.A1 |
| 22 | Consensussequence | Hs.301930.0 | BF057784 | Hs.301930.0.A1 |
| 23 | Consensussequence | Hs.87450.0 | NM_001335.1 | Hs.87450.0.S1 |
| 24 | Consensussequence | Hs.110637.0 | NM_018951.1 | Hs.110637.0_RC |
| 25 | Consensussequence | Hs.36972.0 | AI829961 | Hs.36972.0.S1 |
| 26 | Consensussequence | Hs.179902.1 | NM_022109.1 | Hs.179902.1 |

| | Sequence | | | | |
|---|---|---|---|---|---|
| # | Source | Unigene_Accession | Cluster_Type | LocusLink | Full_length_Reference_Seq |
| 1 | RefSeq | Hs.128155 | fulllength | 29064 | NM_014151; HSPC053 protein |
| 2 | GenBank | Hs.127428 | fulllength | 3205 | NM_002142; homeobox protein A9 isoform b NM_152739; homeobox |
| | | | | | protein A9 isoform a |
| 3 | RefSeq | Hs.98243 | fulllength | 6691 | NM_021114; serine protease inhibitor, Kazal type, 2 (acrosin-trypsin |
| | | | | | inhibitor) |
| 4 | GenBank | Hs.127428 | fulllength | 3205 | NM_002142; homeobox protein A9 isoform b NM_152739; homeobox |
| | | | | | protein A9 isoform a |
| 5 | GenBank | Hs.299916 | fulllength | 257194 | NM_173808; kilon |
| 6 | RefSeq | Hs.98428 | fulllength | 3216 | NM_018952; homeo box B6 isoform 1 NM_156036 |
| | | | | | ; homeo box B6 isoform |
| | | | | | 2 NM_156037; homeo box B6 isoform 1 |
| 7 | GenBank | Hs.110637 | fulllength | 3206 | NM_018951; homeobox protein A10 isoform a NM_153715; |
| | | | | | homeobox protein A10 isoform b |
| 8 | RefSeq | Hs.381039 | fulllength | 27018 | NM_014380; nerve growth factor receptor (TNFRSF16) |
| | | | | | associated protein 1 |
| 9 | RefSeq | Hs.2733 | fulllength | 3212 | NM_002145; homeo box B2 |
| 10 | RefSeq | Hs.61490 | fulllength | 29970 | NM_014575; schwannomin interacting protein 1 |
| 11 | RefSeq | Hs.95594 | fulllength | 54504 | NM_019029; serine carboxypeptidase vitellogenic-like |
| | | | | | NM_031311; serine |
| | | | | | carboxypeptidase vitellogenic-like |
| 12 | GenBank | Hs.248074 | fulllength | 3200 | NM_030661; homeobox A3 protein isoform a |
| | | | | | NM_153631; homeobox A3 |
| | | | | | protein isoform a NM_153632; homeobox A3 protein isoform b |
| 13 | GenBank | Hs.183096 | fulllength | | |
| 14 | GenBank | Hs.169825 | fulllength | 1287 | NM_000495; alpha 5 type IV collagen isoform 1, precursor |
| | | | | | NM_033380; alpha 5 type IV collagen isoform 2, precursor |
| | | | | | NM_033381; alpha 5 type IV collagen isoform 3, precursor |
| 15 | RefSeq | Hs.170177 | fulllength | 4211 | NM_002398; Meis1 homolog |
| 16 | GenBank | Hs.302022 | fulllength | 63976 | NM_022114; PR domain containing 16 |
| 17 | GenBank | Hs.183096 | fulllength | | |
| 18 | GenBank | Hs.445509 | est | | |
| 19 | GenBank | Hs.37034 | fulllength | 3202 | NM_019102; homeobox protein A5 |
| 20 | GenBank | Hs.433379 | | 283683 | |
| 21 | GenBank | Hs.173179 | | | |
| 22 | GenBank | Hs.301930 | fulllength | 221188 | NM_153837; G-protein coupled receptor 114 |
| 23 | GenBank | Hs.87450 | fulllength | 1521 | NM_001335; cathepsin W preproprotein |
| 24 | GenBank | Hs.110637 | fulllength | 3206 | NM_018951; homeobox protein A10 isoform a NM_153715; |
| | | | | | homeobox protein A10 isoform b |
| 25 | GenBank | Hs.36972 | fulllength | 924 | NM_006137; CD7 antigen precursor |
| 26 | GenBank | Hs.179902 | fulllength | 23446 | NM_022109; CDw92 antigen NM_080546; CDw92 antigen |

TABLE 10
genes lower expressed in CEBPA than in t(8 21)

| | | | | | | | Sequence |
|---|---|---|---|---|---|---|---|
| # | affy id | HUGO name | Title | MapLocation | Sequence Type | Transcript ID | Derived From |
| 1 | 228827_at | | Homo sapiens clone 25023 mRNA sequence | | Consensussequence | Hs.90858.0 | AI217416 |
| 2 | 203859_s_at | PALM | paralemmin | 19p13.3 | Exemplarsequence | Hs.78482.0 | NM_002579.1 |
| 3 | 205528_s_at | CBFA2T1 | core-binding factor, runt domain, alpha sub- | 8q22 | Consensussequence | Hs.31551.0 | X79990.1 |
| | | | unit 2; translocated to, 1; cyclin D-related | | | | |
| 4 | 205529_s_at | CBFA2T1 | core-binding factor, runt domain, alpha sub- | 8q22 | Exemplarsequence | Hs.31551.0 | NM_004349.1 |
| | | | unit 2; translocated to, 1; cyclin D-related | | | | |
| 5 | 242845_at | | Homo sapiens mRNA; cDNA DKFZp564B213 (from clone | | Consensussequence | Hs.144995.0 | AI366780 |
| | | | DKFZp564B213) | | | | |
| 6 | 202789_at | | | | Consensussequence | Hs.268177.0 | AL022394 |
| 7 | 206940_s_at | POU4F1 | POU domain, class 4, transcription factor 1 | 13q21.1-q22 | Exemplarsequence | Hs.211588.0 | NM_006237.1 |
| 8 | 235468_at | | ESTs | | Consensussequence | Hs.105805.0 | AA531287 |
| 9 | 233587_s_at | | Homo sapiens cDNA FLJ12790 fis, clone NT2RP2001985, | | Consensussequence | Hs.18760.1 | AK022852.1 |
| | | | weakly similar to Homo sapiens high-risk human papilloma | | | | |
| | | | viruses E6 oncoproteins targeted protein E6TP1 alpha | | | | |
| | | | mRNA. | | | | |
| 10 | 219892_at | TM6SF1 | transmembrane 6 superfamily member 1 | 15q24-q26 | Exemplarsequence | Hs.133865.0 | NM_023003.1 |
| 11 | 225056_at | | Homo sapiens cDNA FLJ12790 fis, clone NT2RP2001985, | | Consensussequence | Hs.18760.0 | AB037810.1 |
| | | | weakly similar to Homo sapiens high-risk human papilloma | | | | |
| | | | viruses E6 oncoproteins targeted protein E6TP1 alpha | | | | |
| | | | mRNA. | | | | |
| 12 | 223046_at | EGLN1 | egl nine homolog 1 (C. elegans) | 1q42.1 | Consensussequence | Hs.6523.1 | NM_022051.1 |
| 13 | 221497_x_at | EGLN1 | egl nine homolog 1 (C. elegans) | 1q42.1 | Exemplarsequence | Hs.6523.1 | BC005369.1 |
| 14 | 211341_at | POU4F1 | POU domain, class 4, transcription factor 1 | 13q21.1-q22 | Exemplarsequence | Hs.211588.1 | L20433.1 |
| 15 | 210512_s_at | VEGF | vascular endothelial growth factor | 6p12 | Exemplarsequence | Hs.73793.0 | AF022375.1 |

| | | Sequence | | | | |
|---|---|---|---|---|---|---|
| # | Sequence ID | Source | Unigene_Accession | Cluster_Type | LocusLink | Full_Length_Reference_Seq |
| 1 | Hs.90858.0.S1 | GenBank | Hs.90858 | | | |
| 2 | g4557041 | RefSeq | Hs.78482 | fulllength | 5064 | NM_002579; paralemmin |
| 3 | Hs.31551.0 | GenBank | Hs.31551 | fulllength | 862 | NM_004349; acute myelogenous leukemia 1 translocation 1 protein isoform MTG8a |
| | | | | | | NM_175634; acute myelogenous leukemia 1 translocation 1 protein isoform MTG8b |
| | | | | | | NM_175635; acute myelogenous leukemia 1 translocation 1 protein isoform MTG8c |
| | | | | | | NM_175636; acute myelogenous leukemia 1 translocation 1 protein isoform MTG8c |
| 4 | g475915 | RefSeq | Hs.31551 | fulllength | 862 | NM_004349; acute myelogenous leukemia 1 translocation 1 protein isoform MTG8a |
| | | | | | | NM_175634; acute myelogenous leukemia 1 translocation 1 protein isoform MTG8b |
| | | | | | | NM_175635; acute myelogenous leukemia 1 translocation 1 protein isoform MTG8c |
| | | | | | | NM_175636; acute myelogenous leukemia 1 translocation 1 protein isoform MTG8c |
| 5 | Hs.144995.0.A1 | GenBank | Hs.380268 | | | |
| 6 | Hs.268177.0.S2 | GenBank | | | | |
| 7 | g5453937 | RefSeq | Hs.211588 | fulllength | 5457 | NM_006237; POU domain, class 4, transcription factor 1 |
| 8 | Hs.105805.0_RC | GenBank | Hs.438798 | est | | |
| 9 | Hs.18760.1 | GenBank | Hs.405863 | fulllength | | |
| 10 | g13194198 | RefSeq | Hs.341203 | fulllength | 53346 | NM_023003; transmembrane 6 superfamily member 1 |
| 11 | Hs.18760.0 | GenBank | Hs.405863 | fulllength | | |
| 12 | Hs.6523.1_RC | GenBank | Hs.6523 | fulllength | 54583 | NM_022051; egl nine homolog 1 |
| 13 | g13529208 | GenBank | Hs.6523 | fulllength | 54583 | NM_022051; egl nine homolog 1 |
| 14 | g418015 | GenBank | Hs.211588 | fulllength | 5457 | NM_006237; POU domain, class 4, transcription factor 1 |
| 15 | g3719220 | GenBank | Hs.73793 | fulllength | 7422 | NM_003376; vascular endothelial growth factor |

TABLE 11
genes higher expressed in CEBPA than in t(15 17)

| | | | | | | Transcript |
|---|---|---|---|---|---|---|
| # | affy id | HUGO name | Title | MapLocation | Sequence Type | ID |
| 1 | 209905_at | HOXA9 | homeo box A9 | 7p15-p14 | Consensussequence | Hs.127428.0 |
| 2 | 214651_s_at | HOXA9 | homeo box A9 | 7p15-p14 | Consensussequence | Hs.127428.2 |
| 3 | 204304_s_at | PROML1 | prominin-like 1 (mouse) | 4p15.33 | Exemplarsequence | Hs.112360.0 |
| 4 | 219054_at | FLJ14054 | hypothetical protein FLJ14054 | 5p13.3 | Exemplarsequence | Hs.13528.0 |
| 5 | 213150_at | HOXA10 | homeo box A10 | 7p15-p14 | Consensussequence | Hs.110637.0 |
| 6 | 204425_at | ARHGAP4 | Rho GTPase activating protein 4 | Xq28 | Exemplarsequence | Hs.3109.0 |
| 7 | 230670_at | FLJ25972 | hypothetical protein FLJ25972 | 3q25.1 | Consensussequence | Hs.88162.0 |
| 8 | 243618_s_at | LOC152485 | hypothetical protein LOC152485 | 4q31.1 | Consensussequence | Hs.229022.0 |
| 9 | 202890_at | MAP7 | microtubule-associated protein 7 | 6q23.2 | Consensussequence | Hs.146388.0 |
| 10 | 211991_s_at | HLA-DPA1 | major histocompatibility complex, class II, DP alpha 1 | 6p21.3 | Consensussequence | Hs.914.0 |
| 11 | 209732_at | CLECSF2 | C-type (calcium dependent, carbohydrate-recognition | 12p13-p12 | Exemplarsequence | Hs.85201.0 |
| | | | domain) lectin, superfamily member 2 (activation-induced) | | | |
| 12 | 235521_at | HOXA3 | homeo box A3 | 7p15-p14 | Consensussequence | Hs.222446.0 |
| 13 | 207269_at | DEFA4 | defensin, alpha 4, corticostatin | 8p23 | Exemplarsequence | Hs.2582.0 |
| 14 | 217388_s_at | KYNU | kynureninase (L-kynurenine hydrolase) | 2q22.1 | Consensussequence | Hs.169139.2 |
| 15 | 219790_s_at | NPR3 | natriuretic peptide receptor C/guanylate cyclase C | 5p14-p13 | Exemplarsequence | Hs.123655.0 |
| | | | (atrionatriuretic peptide receptor C) | | | |
| 16 | 212998_x_at | HLA-DQB1 | major histocompatibility complex, class II, DQ beta 1 | 6p21.3 | Consensussequence | Hs.73931.3 |
| 17 | 226751_at | DKFZP566K1924 | DKFZP566K1924 protein | 2p13.2 | Consensussequence | Hs.26358.0 |
| 18 | 219789_at | NPR3 | natriuretic peptide receptor C/guanylate cyclase C | 5p14-p13 | Consensussequence | Hs.123655.0 |
| | | | (atrionatriuretic peptide receptor C) | | | |
| 19 | 213147_at | HOXA10 | homeo box A10 | 7p15-p14 | Consensussequence | Hs.110637.0 |
| 20 | 201137_s_at | HLA-DPB1 | major histocompatibility complex, class II, DP beta 1 | 6p21.3 | Exemplarsequence | Hs.814.0 |
| 21 | 213537_at | HLA-DPA1 | major histocompatibility complex, class II, DP alpha 1 | 6p21.3 | Consensussequence | Hs.914.1 |

| | Sequence | | | | | | |
|---|---|---|---|---|---|---|---|
| | Derived | | Sequence | | | | |
| # | From | Sequence ID | Source | Unigene_Accession | Cluster_Type | LocusLink | Full_Length_Reference_Seq |
| 1 | AI246769 | Hs.127428.0 | GenBank | Hs.127428 | fulllength | 3205 | NM_002142; homeobox protein A9 isoform |
| | | | | | | | b NM_152739; homeobox protein A9 |
| | | | | | | | isoform a |
| 2 | U41813.1 | Hs.127428.2 | GenBank | Hs.127428 | fulllength | 3205 | NM_002142; homeobox protein A9 isoform |
| | | | | | | | b NM_152739; homeobox protein A9 |
| | | | | | | | isoform a |
| 3 | NM_006017.1 | g5174386 | RefSeq | Hs.112360 | fulllength | 8842 | NM_006017; prominin 1 |
| 4 | NM_024563.1 | g13375730 | RefSeq | Hs.13528 | fulllength | 79614 | NM_024563; hypothetical protein FLJ14054 |
| 5 | NM_018951.1 | Hs.110637.0_RC | GenBank | Hs.110637 | fulllength | 3206 | NM_018951; homeobox protein A10 isoform a |
| | | | | | | | NM_153715; homeobox protein A10 |
| | | | | | | | isoform b |
| 6 | NM_001666.1 | g11386132 | RefSeq | Hs.3109 | fulllength | 393 | NM_001666; Rho GTPase activating protein 4 |
| 7 | AW341661 | Hs.88162.0.A1 | GenBank | Hs.88162 | fulllength | 285313 | NM_178822; hypothetical protein FLJ25972 |
| 8 | BF678830 | Hs.229022.0.A1 | GenBank | Hs.351270 | | 152485 | NM_178835; hypothetical protein LOC152485 |
| 9 | T62571 | Hs.146388.0.S1 | GenBank | Hs.146388 | fulllength | 9053 | NM_003980; microtubule-associated protein 7 |
| 10 | M27487.1 | Hs.914.0_RC | GenBank | Hs.914 | fulllength | 3113 | NM_033554; major histocompatibility |
| | | | | | | | complex, class II, DP alpha 1 precursor |
| 11 | BC005254.1 | g13528920 | GenBank | Hs.85201 | fulllength | 9976 | NM_005127; C-type lectin, superfamily |
| | | | | | | | member 2 |
| 12 | AW137982 | Hs.222446.0.A1 | GenBank | Hs.248074 | fulllength | 3200 | NM_030661; homeobox A3 protein isoform a |
| | | | | | | | NM_153631; homeobox A3 protein isoform a |
| | | | | | | | NM_153632; homeobox A3 protein isoform b |
| 13 | NM_001925.1 | g4503302 | RefSeq | Hs.2582 | fulllength | 1669 | NM_001925; defensin, alpha 4, preproprotein |
| 14 | D55639.1 | Hs.169139.2 | GenBank | Hs.169139 | fulllength | 8942 | NM_003937; kynureninase (L-kynurenine |
| | | | | | | | hydrolase) |
| 15 | NM_000908.1 | g4505440 | RefSeq | Hs.123655 | fulllength | 4883 | NM_000908; natriuretic peptide receptor |
| | | | | | | | C/guanylate cyclase C (atrionatriuretic |
| | | | | | | | peptide receptor C) |
| 16 | AI583173 | Hs.73931.3_RC | GenBank | Hs.73931 | fulllength | 3119 | NM_002123; major histocompatibility |
| | | | | | | | complex, class II, DQ beta 1 precursor |
| 17 | AW193693 | Hs.26358.0.S1 | GenBank | Hs.26358 | fulllength | 25927 | NM_015463; DKFZP566K1924 protein |
| 18 | AI628360 | Hs.123655.0 | GenBank | Hs.123655 | fulllength | 4883 | NM_000908; natriuretic peptide receptor |
| | | | | | | | C/guanylate cyclase C (atrionatriuretic peptide |
| | | | | | | | receptor C) |
| 19 | NM_018951.1 | Hs.110637.0_RC | GenBank | Hs.110637 | fulllength | 3206 | NM_018951; homeobox protein A10 isoform a |
| | | | | | | | NM_153715; homeobox protein A10 |
| | | | | | | | isoform b |
| 20 | NM_002121.1 | g4504404 | RefSeq | Hs.814 | fulllength | 3115 | NM_002121; major histocompatibility |
| | | | | | | | complex, class II, DP beta 1 precursor |
| 21 | AI128225 | Hs.914.1.A1 | GenBank | Hs.914 | fulllength | 3113 | NM_033554; major histocompatibility |
| | | | | | | | complex, class II, DP alpha 1 precursor |

TABLE 12
genes lower expressed in CEBPA than in t(15 17)

| | | | | | | | Sequence |
|---|---|---|---|---|---|---|---|
| # | affy id | HUGO name | Title | MapLocation | Sequence Type | Transcript ID | Derived From |
| 1 | 38487_at | STAB1 | stabilin 1 | 3p21.31 | Consensussequence | 4 | D87433 |
| 2 | 212509_s_at | | ESTs | | Consensussequence | Hs.250723.2 | BF968134 |
| 3 | 200654_at | P4HB | procollagen-proline, 2-oxoglutarate 4- | 17q25 | Exemplarsequence | Hs.75655.0 | J02783.1 |
| | | | dioxygenase (proline 4-hydroxylase), beta | | | | |
| | | | polypeptide (protein disulfide isomerase; | | | | |
| | | | thyroid hormone binding protein p55) | | | | |
| 4 | 204150_at | STAB1 | stabilin 1 | 3p21.31 | Exemplarsequence | Hs.301989.0 | NM_015136.1 |
| 5 | 227326_at | | Homo sapiens cDNA FLJ39789 fis, clone | | Consensussequence | Hs.11924.0 | BE966768 |
| | | | SPLEN2003160. | | | | |
| 6 | 216320_x_at | | | | Consensussequence | Hs.278657.2 | U37055 |
| 7 | 205614_x_at | MST1 | macrophage stimulating 1 (hepatocyte | 3p21 | Exemplarsequence | Hs.278657.0 | NM_020998.1 |
| | | | growth factor-like) | | | | |
| 8 | 205663_at | PCBP3 | poly(rC) binding protein 3 | 21q22.3 | Exemplarsequence | Hs.121241.0 | NM_020528.1 |
| 9 | 200953_s_at | CCND2 | cyclin D2 | 12p13 | Exemplarsequence | Hs.75586.0 | NM_001759.1 |
| 10 | 212953_x_at | CALR | calreticulin | 19p13.3-p13.2 | Consensussequence | Hs.16488.2 | BE251303 |
| 11 | 233072_at | KIAA1857 | netrin G2 | 9q34 | Consensussequence | Hs.163642.0 | AI348745 |
| 12 | 200951_s_at | CCND2 | cyclin D2 | 12p13 | Consensussequence | Hs.75586.0 | NM_001759.1 |
| 13 | 200986_at | SERPING1 | serine (or cysteine) proteinase inhibitor, | 11q12-q13.1 | Exemplarsequence | Hs.151242.0 | NM_000062.1 |
| | | | clade G (C1 inhibitor), member 1, | | | | |
| | | | (angioedema, hereditary) | | | | |
| 14 | 227046_at | C17orf26 | chromosome 17 open reading frame 26 | 17q25.1 | Consensussequence | Hs.3402.0 | BF062384 |
| 15 | 210755_at | HGF | hepatocyte growth factor (hepapoietin A; | 7q21.1 | Exemplarsequence | Hs.809.2 | U46010.1 |
| | | | scatter factor) | | | | |
| 16 | 236787_at | | ESTs | | Consensussequence | Hs.126630.0 | AW591809 |
| 17 | 201666_at | TIMP1 | tissue inhibitor of metalloproteinase 1 | Xp11.3-p11.23 | Exemplarsequence | Hs.5831.0 | NM_003254.1 |
| | | | (erythroid potentiating activity, | | | | |
| | | | collagenase inhibitor) | | | | |
| 18 | 208852_s_at | CANX | calnexin | 5q35 | Consensussequence | Hs.155560.0 | AI761759 |

| # | Sequence ID | Sequence Source | Unigene_Accession | Cluster_Type | LocusLink | Full_Length_Reference_Seq |
|---|---|---|---|---|---|---|
| 1 | 4905477 | GenBank | Hs.301989 | fulllength | 23166 | NM_015136; stabilin 1 |
| 2 | Hs.250723.2.S1 | GenBank | Hs.356623 | est | | |
| 3 | g339646 | GenBank | Hs.410578 | fulllength | 5034 | NM_000918; prolyl 4-hydroxylase, beta subunit |
| 4 | g12225239 | RefSeq | Hs.301989 | fulllength | 23166 | NM_015136; stabilin 1 |
| 5 | Hs.11924.0.A1 | GenBank | Hs.11924 | | | |
| 6 | Hs.278657.2.S1 | GenBank | | | | |
| 7 | g10337614 | RefSeq | Hs.349110 | fulllength | 4485 | NM_020998; macrophage stimulating 1 |
| | | | | | | (hepatocyte growth factor-like) |
| 8 | g10092616 | RefSeq | Hs.121241 | fulllength | 54039 | NM_020528; poly(rC) binding protein 3 |
| 9 | g4502616 | RefSeq | Hs.75586 | fulllength | 894 | NM_001759; cyclin D2 |
| 10 | Hs.16488.2_RC | GenBank | Hs.353170 | fulllength | 811 | NM_004343; calreticulin precursor |
| 11 | Hs.163642.0.S1 | GenBank | Hs.163642 | fulllength | 84628 | NM_032536; netrin G2 |
| 12 | Hs.75586.0_RC | GenBank | Hs.75586 | fulllength | 894 | NM_001759; cyclin D2 |
| 13 | g4557378 | RefSeq | Hs.151242 | fulllength | 710 | NM_000062; complement component 1 inhibitor |
| | | | | | | precursor |
| 14 | Hs.3402.0_RC | GenBank | Hs.3402 | fulllength | 201266 | NM_139177; chromosome 17 open |
| | | | | | | reading frame 26 |
| 15 | g1378041 | GenBank | Hs.809 | fulllength | 3082 | NM_000601; hepatocyte growth factor precursor |
| 16 | Hs.126630.0.A1 | GenBank | Hs.390407 | est | | |
| 17 | g4507508 | RefSeq | Hs.5831 | fulllength | 7076 | NM_003254; tissue inhibitor of |
| | | | | | | metalloproteinase 1 precursor |
| 18 | Hs.155560.0.S2 | GenBank | Hs.155560 | fulllength | 821 | NM_001746; calnexin |

TABLE 13
Schnittger CEBPA application

| | | | | | | | | | | Sequence | |
|---|---|---|---|---|---|---|---|---|---|---|---|
| | | HUGO | | | | | | | | Derived | |
| # | affy id | name | F | p | q | Title | MapLocation | Sequence Type | Transcript ID | From | Sequence ID |
| 1 | 213147_at | HOXA10 | 264.59 | 1.35E−27 | 4.13E−23 | homeo box A10 | 7p15-p14 | Consensussequence | Hs.110637.0 | NM_018951.1 | Hs.110637.0_RC |
| 2 | 214651_s_at | HOXA9 | 263.55 | 6.8E−27 | 1.04E−22 | homeo box A9 | 7p15-p14 | Consensussequence | Hs.127428.2 | U41813.1 | Hs.127428.2 |
| 3 | 205453_at | HOXB2 | 206.96 | 1.82E−24 | 1.86E−20 | homeo box B2 | 17q21-q22 | Exemplarsequence | Hs.2733.0 | NM_002145.1 | g4504464 |
| 4 | 235753_at | | 193.18 | 6.08E−24 | 4.67E−20 | Homo sapiens cDNA FLJ34835 fis, clone | | Consensussequence | Hs.196169.0 | AI492051 | Hs.196169.0 |
| | | | | | | NT2NE2010150. | | | | | |
| 5 | 209905_at | HOXA9 | 177.49 | 4.97E−23 | 2.54E−19 | homeo box A9 | 7p15-p14 | Consensussequence | Hs.127428.0 | AI246769 | Hs.127428.0 |
| 6 | 221581_s_at | WBSCR5 | 169.87 | 4.23E−23 | 2.54E−19 | Williams-Beuren syndrome chromosome | 7q11.23 | Exemplarsequence | Hs.56607.1 | AF257135.1 | g9651998 |
| | | | | | | region 5 | | | | | |
| 7 | 213150_at | HOXA10 | 154.57 | 2.98E−22 | 1.31E−18 | homeo box A10 | 7p15-p14 | Consensussequence | Hs.110637.0 | NM_018951.1 | Hs.110637.0_RC |
| 8 | 206847_s_at | HOXA7 | 137.23 | 2.86E−21 | 1.1E−17 | homeo box A7 | 7p15-p14 | Exemplarsequence | Hs.70954.0 | AF026397.1 | g2739070 |
| 9 | 217963_s_at | NGFRAP1 | 136.47 | 1.03E−20 | 3.15E−17 | nerve growth factor receptor (TNFRSF16) | Xq22.1 | Exemplarsequence | Hs.17775.0 | NM_014380.1 | g7657043 |
| | | | | | | associated protein 1 | | | | | |
| 10 | 213844_at | HOXA5 | 133.66 | 7.02E−21 | 2.39E−17 | homeo box A5 | 7p15-p14 | Consensussequence | Hs.37034.0 | NM_019102.1 | Hs.37034.0.S1 |
| 11 | 227853_at | | 115.9 | 2.39E−20 | 6.67E−17 | ESTs, Weakly similar to I60307 beta- | | Consensussequence | Hs.279860.1 | AW024350 | Hs.279860.1.S1 |
| | | | | | | galactosidase, alpha peptide - Escherichia | | | | | |
| | | | | | | coli [E. coli] | | | | | |
| 12 | 235521_at | HOXA3 | 115.62 | 3.73E−19 | 8.17E−16 | homeo box A3 | 7p15-p14 | Consensussequence | Hs.222446.0 | AW137982 | Hs.222446.0.A1 |
| 13 | 233467_s_at | PHEMX | 113.32 | 2.43E−19 | 5.73E−16 | pan-hematopoietic expression | 11p15.5 | Consensussequence | Hs.271954.2 | AF176071.1 | Hs.271954.2 |
| 14 | 205366_s_at | HOXB6 | 112.34 | 6.85E−19 | 1.11E−15 | homeo box B6 | 17q21.3 | Exemplarsequence | Hs.98428.0 | NM_018952.1 | g9506792 |
| 15 | 205601_s_at | HOXB5 | 108.77 | 4.84E−19 | 8.75E−16 | homeo box B5 | 17q21.3 | Exemplarsequence | Hs.22554.0 | NM_002147.1 | g4504468 |
| 16 | 243806_at | | 108.34 | 4.46E−19 | 8.75E−16 | ESTs | | Consensussequence | Hs.161723.0 | AW015140 | Hs.161723.0.A1 |
| 17 | 228827_at | | 107.38 | 4.62E−19 | 8.75E−16 | Homo sapiens clone 25023 mRNA sequence | | Consensussequence | Hs.90858.0 | AI217416 | Hs.90858.0.S1 |
| 18 | 208091_s_at | DKFZP564K0822 | 105.18 | 1.69E−19 | 4.33E−16 | hypothetical protein DKFZp564K0822 | 7p14.1 | Exemplarsequence | g13540577 | NM_030796.1 | g13540577 |
| 19 | 225615_at | LOC126917 | 104.42 | 6.23E−19 | 1.06E−15 | hypothetical protein LOC126917 | 1p36.13 | Consensussequence | Hs.13766.0 | AK024480.1 | Hs.13766.0 |
| 20 | 205600_x_at | HOXB5 | 101.3 | 1.03E−18 | 1.51E−15 | homeo box B5 | 17q21.3 | Consensussequence | Hs.22554.0 | AI052747 | Hs.22554.0.S1 |
| 21 | 236892_s_at | | 101.27 | 5.04E−18 | 5.67E−15 | Homo sapiens, clone MGC: 10077 | | Consensussequence | Hs.269918.0 | BF590528 | Hs.269918.0.A1 |
| | | | | | | IMAGE: 3896690, mRNA, complete cds | | | | | |
| 22 | 228904_at | | 101.16 | 5.17E−18 | 5.67E−15 | ESTs | | Consensussequence | Hs.156044.0 | AW510657 | Hs.156044.0 |
| 23 | 227279_at | MGC15737 | 100.41 | 8.48E−19 | 1.3E−15 | hypothetical protein MGC15737 | Xq22.1 | Consensussequence | Hs.39122.0 | AA847654 | Hs.39122.0.S1 |
| 24 | 230894_s_at | | 99.92 | 2.89E−18 | 3.86E−15 | Homo sapiens, clone IMAGE: 4154313, | | Consensussequence | Hs.42640.1 | BE672557 | Hs.42640.1.A1 |
| | | | | | | mRNA, partial cds | | | | | |
| 25 | 215087_at | | 97.93 | 3.2E−18 | 4.09E−15 | Homo sapiens mRNA full length insert cDNA | | Consensussequence | Hs.306331.0 | AL109730.1 | Hs.306331.0 |
| | | | | | | clone EUROIMAGE 68600. | | | | | |
| 26 | 228365_at | LOC144402 | 97.7 | 5.75E−18 | 6.09E−15 | copine VIII | 12q11 | Consensussequence | Hs.71818.0 | AI765180 | Hs.71818.0.A1 |
| 27 | 203949_at | MPO | 96.45 | 7E−18 | 7.16E−15 | myeloperoxidase | 17q23.1 | Exemplarsequence | Hs.1817.0 | NM_000250.1 | g4557758 |
| 28 | 203017_s_at | SSX2IP | 95.93 | 2.45E−18 | 3.42E−15 | synovial sarcoma, X breakpoint 2 interacting | | Consensussequence | Hs.22587.0 | AW136988 | Hs.22587.0.S1 |
| | | | | | | protein | | | | | |
| 29 | 239791_at | | 93.94 | 1.84E−17 | 1.66E−14 | Homo sapiens, clone MGC: 10077 | | Consensussequence | Hs.269918.1 | AI125255 | Hs.269918.1.A1 |
| | | | | | | IMAGE: 3896690, mRNA, complete cds | | | | | |
| 30 | 233955_x_at | HSPC195 | 93.61 | 4.4E−18 | 5.19E−15 | hypothetical protein HSPC195 | 5q31.3 | Consensussequence | Hs.15093.1 | AK001782.1 | Hs.15093.1 |
| 31 | 217975_at | LOC51186 | 92.94 | 1.07E−17 | 1.02E−14 | pp21 homolog | Xq22.1 | Exemplarsequence | Hs.15984.0 | NM_016303.1 | g10047099 |
| 32 | 206310_at | SPINK2 | 92.14 | 3.36E−17 | 2.4E−14 | serine protease inhibitor, Kazal type, 2 | 4q12 | Exemplarsequence | Hs.98243.0 | NM_021114.1 | g10863910 |
| | | | | | | (acrosin-trypsin inhibitor) | | | | | |
| 33 | 204069_at | MEIS1 | 90.91 | 4.11E−17 | 2.81E−14 | Meis1, myeloid ecotropic viral integration site | 2p14-p13 | Exemplarsequence | Hs.170177.0 | NM_002398.1 | g4505150 |
| | | | | | | 1 homolog (mouse) | | | | | |
| 34 | 238077_at | MGC27385 | 90.29 | 3.56E−18 | 4.37E−15 | hypothetical protein MGC27385 | 3p21.1 | Consensussequence | Hs.13982.1 | T75480 | Hs.13982.1_RC |
| 35 | 224764_at | ARHGAP10 | 89.42 | 8.97E−18 | 8.88E−15 | Rho-GTPase activating protein 10 | 10 | Consensussequence | Hs.11611.0 | AB037845.1 | Hs.11611.0.A1 |
| 36 | 216417_x_at | HOXB9 | 87.63 | 3.06E−17 | 2.35E−14 | homeo box B9 | 17q21.3 | Consensussequence | Hs.287809.0 | X16172 | Hs.287809.0.S1 |
| 37 | 238012_at | | 87.48 | 1.5E−17 | 1.39E−14 | Homo sapiens, Similar to mannosidase, | | Consensussequence | Hs.37916.0 | AI620209 | Hs.37916.0_RC |
| | | | | | | alpha, class 1B, member 1, clone | | | | | |
| | | | | | | IMAGE: 3623379, mRNA | | | | | |
| 38 | 241706_at | LOC144402 | 86.85 | 2.67E−17 | 2.1E−14 | copine VIII | 12q11 | Consensussequence | Hs.98760.0 | AA431782 | Hs.98760.0.A1 |
| 39 | 231767_at | HOXB4 | 84.6 | 5.83E−17 | 3.89E−14 | homeo box B4 | 17q21-q22 | Consensussequence | Hs.126666.0 | AL137449.1 | Hs.126666.0 |
| 40 | 229971_at | GPR114 | 83.72 | 9.09E−17 | 5.81E−14 | G protein-coupled receptor 114 | 16q12.2 | Consensussequence | Hs.301930.0 | BF057784 | Hs.301930.0.A1 |
| 41 | 204202_at | KIAA1023 | 83.18 | 3.21E−17 | 2.4E−14 | KIAA1023 protein | 7p22.3 | Exemplarsequence | Hs.21361.0 | NM_017604.1 | g8922140 |
| 42 | 226865_at | | 82.7 | 6.24E−17 | 4.08E−14 | ESTs, Moderately similar to hypothetical | | Consensussequence | Hs.99472.1 | AW130600 | Hs.99472.1_RC |
| | | | | | | protein FLJ20378 [Homo sapiens] | | | | | |
| | | | | | | [H. sapiens] | | | | | |
| 43 | 201952_at | ALCAM | 81.76 | 1.93E−17 | 1.69E−14 | activated leukocyte cell adhesion molecule | 3q13.1 | Consensussequence | Hs.10247.0 | NM_001627.1 | Hs.10247.0 |
| 44 | 208146_s_at | CPVL | 81.1 | 3.11E−16 | 1.65E−13 | carboxypeptidase, vitellogenic-like | 7p15-p14 | Exemplarsequence | g13786124 | NM_031311.1 | g13786124 |
| 45 | 241370_at | | 80.83 | 1.23E−16 | 7.53E−14 | Homo sapiens cDNA FLJ37785 fis, clone | | Consensussequence | Hs.100691.0 | AA278233 | Hs.100691.0_RC |
| | | | | | | BRHIP2028330. | | | | | |
| 46 | 213908_at | | 80.82 | 2.17E−16 | 1.25E−13 | Homo sapiens, clone IMAGE: 4837016, | | Consensussequence | Hs.295446.0 | AI824078 | Hs.295446.0.A1 |
| | | | | | | mRNA | | | | | |
| 47 | 238604_at | | 80.31 | 2.13E−17 | 1.82E−14 | Homo sapiens cDNA FLJ25559 fis, clone | | Consensussequence | Hs.140489.0 | AA768884 | Hs.140489.0.A1 |
| | | | | | | JTH02834. | | | | | |
| 48 | 214450_at | CTSW | 79.48 | 2.8E−16 | 1.54E−13 | cathepsin W (lymphopain) | 11q13.1 | Consensussequence | Hs.87450.0 | NM_001335.1 | Hs.87450.0.S1 |
| 49 | 203680_at | PRKAR2B | 78.35 | 2.02E−16 | 1.19E−13 | protein kinase, cAMP-dependent, regulatory, | 7q22-q31.1 | Exemplarsequence | Hs.77439.0 | NM_002736.1 | g4506064 |
| | | | | | | type II, beta | | | | | |
| 50 | 213110_s_at | COL4A5 | 78.29 | 6.28E−16 | 3.16E−13 | collagen, type IV, alpha 5 (Alport syndrome) | Xq22 | Consensussequence | Hs.169825.0 | AW052179 | Hs.169825.0_RC |
| 51 | 222996_s_at | HSPC195 | 78.25 | 2E−16 | 1.19E−13 | hypothetical protein HSPC195 | 5q31.3 | Exemplarsequence | Hs.15093.0 | BC002490.1 | g12803342 |
| 52 | 226134_s_at | | 77.62 | 4.23E−16 | 2.2E−13 | Homo sapiens, clone IMAGE: 4154313, | | Consensussequence | Hs.42640.0 | AI978754 | Hs.42640.0.A1 |
| | | | | | | mRNA, partial cds | | | | | |
| 53 | 202732_at | PKIG | 77.36 | 2.48E−16 | 1.41E−13 | protein kinase (cAMP-dependent, catalytic) | 20q12-q13.1 | Exemplarsequence | Hs.3407.0 | NM_007066.1 | g5902019 |
| | | | | | | inhibitor gamma | | | | | |
| 54 | 224593_at | DKFZp761B128 | 76.84 | 3.78E−17 | 2.63E−14 | hypothetical protein DKFZp761B128 | 12q24.31 | Consensussequence | Hs.61976.0 | BE965646 | Hs.61976.0.S1 |
| 55 | 240572_s_at | | 76.82 | 2.42E−17 | 2.01E−14 | Homo sapiens cDNA FLJ38955 fis, clone | | Consensussequence | Hs.156100.1 | BF436632 | Hs.156100.1.A1 |
| | | | | | | NT2RI2000107. | | | | | |
| 56 | 212895_s_at | ABR | 76.37 | 1.14E−16 | 7.13E−14 | active BCR-related gene | 17p13.3 | Consensussequence | Hs.118021.2 | AL527773 | Hs.118021.2_RC |
| 57 | 220560_at | C11orf21 | 75.03 | 2.57E−17 | 2.07E−14 | chromosome 11 open reading frame 21 | 11p15.5 | Exemplarsequence | Hs.272100.0 | NM_014144.1 | g7662662 |
| 58 | 208890_s_at | PLXNB2 | 74.34 | 3.3E−17 | 2.4E−14 | plexin B2 | 22q13.33 | Exemplarsequence | Hs.3989.0 | BC004542.1 | g13528689 |
| 59 | 225240_s_at | | 73.13 | 1.48E−15 | 6.49E−13 | Homo sapiens, clone IMAGE: 4154313, | | Consensussequence | Hs.42179.0 | BF435123 | Hs.42179.0.A1 |
| | | | | | | mRNA, partial cds | | | | | |
| 60 | 201951_at | ALCAM | 73.04 | 3.06E−16 | 1.65E−13 | activated leukocyte cell adhesion molecule | 3q13.1 | Consensussequence | Hs.10247.0 | NM_001627.1 | Hs.10247.0 |
| 61 | 212314_at | KIAA0746 | 72.58 | 1.14E−15 | 5.29E−13 | KIAA0746 protein | 4p15.2 | Consensussequence | Hs.49500.0 | AB018289.1 | Hs.49500.0 |
| 62 | 220558_x_at | PHEMX | 72.33 | 2E−15 | 8.18E−13 | pan-hematopoietic expression | 11p15.5 | Exemplarsequence | Hs.271954.0 | NM_005705.1 | g5032206 |
| 63 | 238778_at | FLJ32798 | 72.22 | 1.09E−15 | 5.13E−13 | hypothetical protein FLJ32798 | 10p11.1 | Consensussequence | Hs.103296.0 | AI244661 | Hs.103296.0 |
| 64 | 223398_at | MGC11115 | 71.65 | 2.64E−16 | 1.47E−13 | hypothetical protein MGC11115 | 9q22.2 | Exemplarsequence | Hs.39132.0 | BC004500.1 | g13325387 |
| 65 | 204495_s_at | DKFZP434H132 | 70.9 | 8.45E−16 | 4.12E−13 | DKFZP434H132 protein | 15q22.33 | Exemplarsequence | Hs.17936.0 | NM_015492.1 | g7661575 |
| 66 | 238756_at | | 70.45 | 1.32E−15 | 5.94E−13 | Homo sapiens cDNA FLJ35212 fis, clone | | Consensussequence | Hs.41294.0 | AI860012 | Hs.41294.0_RC |
| | | | | | | PROST1000136. | | | | | |
| 67 | 212311_at | KIAA0746 | 70.22 | 1.56E−15 | 6.63E−13 | KIAA0746 protein | 4p15.2 | Consensussequence | Hs.49500.0 | AB018289.1 | Hs.49500.0 |
| 68 | 204494_s_at | DKFZP434H132 | 70.17 | 5.14E−16 | 2.63E−13 | DKFZP434H132 protein | 15q22.33 | Consensussequence | Hs.17936.0 | AW516789 | Hs.17936.0 |
| 69 | 213940_s_at | FNBP1 | 70.17 | 9.37E−16 | 4.49E−13 | formin binding protein 1 | 9q34 | Consensussequence | Hs.301763.1 | AU145053 | Hs.301763.1.S1 |
| 70 | 204082_at | PBX3 | 69.27 | 1.24E−15 | 5.68E−13 | pre-B-cell leukemia transciption factor 3 | 9q33-q34 | Exemplarsequence | Hs.294101.0 | NM_006195.1 | g5453851 |
| 71 | 219062_s_at | FLJ20281 | 68.69 | 1.53E−15 | 6.61E−13 | hypothetical protein FLJ20281 | 18q21.32 | Exemplarsequence | Hs.18800.0 | NM_017742.1 | g8923259 |
| 72 | 201243_s_at | ATP1B1 | 68.07 | 1.73E−15 | 7.28E−13 | ATPase, Na+/K+ transporting, beta 1 | 1q22-q25 | Exemplarsequence | Hs.78629.0 | NM_001677.1 | g4502276 |
| | | | | | | polypeptide | | | | | |
| 73 | 226206_at | FLJ32205 | 67.95 | 4.3E−15 | 1.61E−12 | hypothetical protein FLJ32205 | 7p22.3 | Consensussequence | Hs.11607.0 | BG231691 | Hs.11607.0.A1 |
| 74 | 217226_s_at | BA108L7.2 | 67.94 | 2.95E−15 | 1.19E−12 | similar to rat tricarboxylate carrier-like protein | 10q24.31 | Consensussequence | Hs.155606.2 | M95929.1 | Hs.155606.2.S1 |
| 75 | 218450_at | HEBP1 | 67.86 | 1.89E−15 | 7.83E−13 | heme binding protein 1 | 12p13.2 | Exemplarsequence | Hs.108675.0 | NM_015987.1 | g7705404 |
| 76 | 207839_s_at | LOC51754 | 67.53 | 8.1E−15 | 2.86E−12 | NAG-5 protein | 9p13.1 | Exemplarsequence | Hs.8087.0 | NM_016446.1 | g7706546 |
| 77 | 215440_s_at | FLJ10097 | 67.35 | 4.37E−15 | 1.62E−12 | hypothetical protein FLJ10097 | Xq22.1-q22.3 | Consensussequence | Hs.184736.1 | AL523320 | Hs.184736.1.A1 |
| 78 | 203741_s_at | ADCY7 | 66.97 | 1.46E−15 | 6.49E−13 | adenylate cyclase 7 | 16q12-q13 | Exemplarsequence | Hs.172199.0 | NM_001114.1 | g4557254 |
| 79 | 215051_x_at | AIF1 | 66.93 | 7.12E−16 | 3.52E−13 | allograft inflammatory factor 1 | 6p21.3 | Consensussequence | Hs.76364.4 | BF213829 | Hs.76364.4 |
| 80 | 209500_x_at | TNFSF13 | 66.73 | 3.51E−15 | 1.38E−12 | tumor necrosis factor (ligand) superfamily, | 17p13.1 | Exemplarsequence | Hs.54673.2 | AF114012.1 | g7328555 |
| | | | | | | member 13 | | | | | |
| 81 | 220974_x_at | BA108L7.2 | 66.5 | 4.95E−15 | 1.81E−12 | similar to rat tricarboxylate carrier-like protein | 10q24.31 | Exemplarsequence | g13569945 | NM_030971.1 | g13569945 |
| 82 | 224516_s_at | HSPC195 | 66.33 | 3.29E−15 | 1.31E−12 | hypothetical protein HSPC195 | 5q31.3 | Exemplarsequence | g13623618 | BC006428.1 | g13623618 |
| 83 | 225010_at | D10S170 | 66.08 | 7.47E−15 | 2.67E−12 | DNA segment on chromosome 10 (unique) | 10q21 | Consensussequence | Hs.288862.0 | AK024913.1 | Hs.288862.0.A1 |
| | | | | | | 170 | | | | | |
| 84 | 206289_at | HOXA4 | 65.48 | 6.35E−15 | 2.29E−12 | homeo box A4 | 7p15-p14 | Exemplarsequence | Hs.77637.0 | NM_002141.1 | g4504458 |
| 85 | 204785_x_at | IFNAR2 | 65.37 | 3.81E−15 | 1.48E−12 | interferon (alpha, beta and omega) receptor 2 | 21q22.11 | Exemplarsequence | Hs.86958.0 | NM_000874.1 | g4504600 |
| 86 | 243010_at | MSI2 | 65.28 | 1E−14 | 3.38E−12 | musashi homolog 2 (Drosophila) | 17q23.1 | Consensussequence | Hs.103512.0 | BE000929 | Hs.103512.0.A1 |
| 87 | 203948_s_at | MPO | 64.76 | 3.9E−14 | 1.12E−11 | myeloperoxidase | 17q23.1 | Exemplarsequence | Hs.1817.0 | J02694.1 | g189039 |
| 88 | 205518_s_at | CMAH | 63.14 | 8.5E−15 | 2.96E−12 | cytidine monophosphate-N-acetylneuraminic | 6p21.32 | Exemplarsequence | Hs.24697.0 | NM_003570.1 | g4502908 |
| | | | | | | acid hydroxylase (CMP-N-acetylneuraminate | | | | | |
| | | | | | | monooxygenase) | | | | | |
| 89 | 237189_at | HOXB2 | 62.36 | 1.74E−14 | 5.62E−12 | homeo box B2 | 17q21-q22 | Consensussequence | Hs.124020.0 | BF060978 | Hs.124020.0.A1 |
| 90 | 205528_s_at | CBFA2T1 | 62.36 | 7.36E−14 | 1.87E−11 | core-binding factor, runt domain, alpha | 8q22 | Consensussequence | Hs.31551.0 | X79990.1 | Hs.31551.0 |
| | | | | | | subunit 2; translocated to, 1; cyclin D-related | | | | | |
| 91 | 213385_at | CHN2 | 62.32 | 1.05E−14 | 3.5E−12 | chimerin (chimaerin) 2 | 7p15.3 | Consensussequence | Hs.286055.2 | AK026415.1 | Hs.286055.2 |
| 92 | 238455_at | | 62.28 | 9.3E−15 | 3.21E−12 | ESTs | | Consensussequence | Hs.72639.0 | AA329676 | Hs.72639.0_RC |
| 93 | 227556_at | ATP1B1 | 61.88 | 1.84E−14 | 5.88E−12 | ATPase, Na+/K+ transporting, beta 1 | 1q22-q25 | Consensussequence | Hs.78629.2 | AI094580 | Hs.78629.2.A1 |
| | | | | | | polypeptide | | | | | |
| 94 | 228345_at | | 61.07 | 3.69E−14 | 1.07E−11 | ESTs, Moderately similar to cystein-rich | | Consensussequence | Hs.34656.0 | AI745136 | Hs.34656.0.A1 |
| | | | | | | hydrophobic domain 2; BRX-like-translocated | | | | | |
| | | | | | | in leukemia; BRX-like translocated in | | | | | |
| | | | | | | leukemia; cysteine-rich hydrophobic 2 [Homo | | | | | |
| | | | | | | sapiens] [H. sapiens] | | | | | |
| 95 | 202006_at | PTPN12 | 61.07 | 4.31E−15 | 1.61E−12 | protein tyrosine phosphatase, non-receptor | 7q11.23 | Exemplarsequence | Hs.62.0 | NM_002835.1 | g4506286 |
| | | | | | | type 12 | | | | | |
| 96 | 213408_s_at | MGC14697 | 60.92 | 9.74E−15 | 3.32E−12 | hypothetical protein MGC14697 | 10q24.32 | Consensussequence | Hs.171625.3 | AK024034.1 | Hs.171625.3 |
| 97 | 207081_s_at | PIK4CA | 60.3 | 1.61E−14 | 5.26E−12 | phosphatidylinositol 4-kinase, catalytic, alpha | 22q11.21 | Exemplarsequence | Hs.171625.0 | NM_002650.1 | g4505806 |
| | | | | | | polypeptide | | | | | |
| 98 | 235749_at | UGCGL2 | 60.11 | 1.51E−14 | 5E−12 | UDP-glucose ceramide glucosyltransferase- | 13q32.1 | Consensussequence | Hs.133423.0 | AI057619 | Hs.133423.0.A1 |
| | | | | | | like 2 | | | | | |
| 99 | 210314_x_at | TNFSF13 | 60.07 | 2.09E−14 | 6.61E−12 | tumor necrosis factor (ligand) superfamily, | 17p13.1 | Exemplarsequence | Hs.54673.1 | AF114013.1 | g7328557 |
| | | | | | | member 13 | | | | | |
| 100 | 206940_s_at | POU4F1 | 60.03 | 4.28E−12 | 7.02E−10 | POU domain, class 4, transcription factor 1 | 13q21.1-q22 | Exemplarsequence | Hs.211588.0 | NM_006237.1 | g5453937 |

TABLE 13
Schnittger CEBPA application

| | Sequence | | | Locus- | |
|---|---|---|---|---|---|
| # | Source | Unigene_Accession | Cluster_Type | Link | Full_Length_Reference_Seq |
| 1 | GenBank | Hs.110637 | fulllength | 3206 | NM_018951; homeobox protein A10 isoform a NM_153715; homeobox protein |
| | | | | | A10 isoform b |
| 2 | GenBank | Hs.127428 | fulllength | 3205 | NM_002142; homeobox protein A9 isoform b NM_152739; homeobox protein A9 |
| | | | | | isoform a |
| 3 | RefSeq | Hs.2733 | fulllength | 3212 | NM_002145; homeo box B2 |
| 4 | GenBank | Hs.196169 | | | |
| 5 | GenBank | Hs.127428 | fulllength | 3205 | NM_002142; homeobox protein A9 isoform b NM_152739; homeobox protein A9 |
| | | | | | isoform a |
| 6 | GenBank | Hs.56607 | fulllength | 7462 | NM_014146; WBSCR5 protein isoform 1 NM_022040; WBSCR5 protein isoform |
| | | | | | 1 NM_032463; WBSCR5 protein isoform 1 NM_032464; WBSCR5 protein |
| | | | | | isoform 2 |
| 7 | GenBank | Hs.110637 | fulllength | 3206 | NM_018951; homeobox protein A10 isoform a NM_153715; homeobox protein |
| | | | | | A10 isoform b |
| 8 | GenBank | Hs.446318 | fulllength | 3204 | NM_006896; homeobox protein A7 |
| 9 | RefSeq | Hs.381039 | fulllength | 27018 | NM_014380; nerve growth factor receptor (TNFRSF16) associated protein 1 |
| 10 | GenBank | Hs.37034 | fulllength | 3202 | NM_019102; homeobox protein A5 |
| 11 | GenBank | Hs.356538 | est | | |
| 12 | GenBank | Hs.248074 | fulllength | 3200 | NM_030661; homeobox A3 protein isoform a NM_153631; homeobox A3 protein |
| | | | | | isoform a NM_153632; homeobox A3 protein isoform b |
| 13 | GenBank | Hs.271954 | fulllength | 10077 | NM_005705: tumor-suppressing subtransferable candidate 6 isoform 2 |
| | | | | | NM_139022; tumor-suppressing subtransferable candidate 6 isoform 1 |
| | | | | | NM_139023; tumor-suppressing subtransferable candidate 6 isoform 4 |
| | | | | | NM_139024; tumor-suppressing subtransferable candidate 6 isoform 3 |
| 14 | RefSeq | Hs.98428 | fulllength | 3216 | NM_018952; homeo box B6 isoform 1 NM_156036; homeo box B6 isoform 2 |
| | | | | | NM_156037; homeo box B6 isoform 1 |
| 15 | RefSeq | Hs.22554 | fulllength | 3215 | NM_002147: homeo box B5 |
| 16 | GenBank | Hs.443007 | est | | |
| 17 | GenBank | Hs.90858 | | | |
| 18 | RefSeq | Hs.4750 | fulllength | 81552 | NM_030796; hypothetical protein DKFZp564K0822 |
| 19 | GenBank | Hs.13766 | | 126917 | |
| 20 | GenBank | Hs.22554 | fulllength | 3215 | NM_002147; homeo box B5 |
| 21 | GenBenk | Hs.183096 | fulllength | | |
| 22 | GenBank | Hs.156044 | est | | |
| 23 | GenBank | Hs.39122 | fulllength | 85012 | NM_032926; hypothetical protein MGC15737 |
| 24 | GenBank | Hs.173179 | | | |
| 25 | GenBank | Hs.306331 | | | |
| 26 | GenBank | Hs.71818 | fulllength | 144402 | NM_153634; copine VIII |
| 27 | RefSeq | Hs.1817 | fulllength | 4353 | NM_000250; myeloperoxidase |
| 28 | GenBank | Hs.22587 | fulllength | 117178 | NM_014021; synovial sarcoma, X breakpoint 2 interacting protein |
| 29 | GenBank | Hs.183096 | fulllength | | |
| 30 | GenBank | Hs.15093 | fulllength | 51523 | NM_016463; hypothetical protein HSPC195 |
| 31 | RefSeq | Hs.15984 | fulllength | 51186 | NM_016303; pp21 homolog |
| 32 | RefSeq | Hs.98243 | fulllength | 6691 | NM_021114; serine protease inhibitor, Kazal type, 2 (acrosin-trypsin inhibitor) |
| 33 | RefSeq | Hs.170177 | fulllength | 4211 | NM_002398; Meis1 homolog |
| 34 | GenBank | Hs.13982 | fulllength | 200845 | NM_153331; hypothetical protein MGC27385 |
| 35 | GenBank | Hs.11611 | fulllength | 57584 | NM_020824; Rho-GTPase activating protein 10 |
| 36 | GenBank | Hs.86327 | fulllength | 3219 | NM_024017; homeo box B9 |
| 37 | GenBank | Hs.37916 | | | |
| 38 | GenBank | Hs.71818 | fulllength | 144402 | NM_153634; copine VIII |
| 39 | GenBank | Hs.126666 | fulllength | 3214 | NM_024015; homeo box B4 |
| 40 | GenBank | Hs.301930 | fulllength | 221188 | NM_153837; G-protein coupled receptor 114 |
| 41 | RefSeq | Hs.21361 | fulllength | 23288 | NM_017604; NM_152558; hypothetical protein DKFZp434I0118 |
| 42 | GenBank | Hs.99472 | est | | |
| 43 | GenBank | Hs.10247 | fulllength | 214 | NM_001627; activated leukocyte cell adhesion molecule |
| 44 | RefSeq | Hs.95594 | fulllength | 54504 | NM_019029; serine carboxypeptidase vitellogenic-like NM_031311; serine |
| | | | | | carboxypeptidase vitellogenic-like |
| 45 | GenBank | Hs.100691 | | | |
| 46 | GenBank | Hs.362800 | | | |
| 47 | GenBank | Hs.140489 | | | |
| 48 | GenBank | Hs.87450 | fulllength | 1521 | NM_001335; cathepsin W preproprotein |
| 49 | RefSeq | Hs.77439 | fulllength | 5577 | NM_002736; protein kinase, cAMP-dependent, regulatory, type II, beta |
| 50 | GenBank | Hs.169825 | fulllength | 1287 | NM_000495; alpha 5 type IV collagen isoform 1, precursor NM_033380; alpha 5 |
| | | | | | type IV collagen isoform 2, precursor NM_033381; alpha 5 type IV collagen |
| | | | | | isoform 3, precursor |
| 51 | GenBank | Hs.15093 | fulllength | 51523 | NM_016463; hypothetical protein HSPC195 |
| 52 | GenBank | Hs.1731709 | | | |
| 53 | RefSeq | Hs.3407 | fulllength | 11142 | NM_007066; protein kinase (cAMP-dependent, catalytic) inhibitor gamma |
| 54 | GenBank | Hs.61976 | fulllength | 144348 | NM_152437; hypothetical protein DKFZp761B128 |
| 55 | GenBank | Hs.156100 | est | | |
| 56 | GenBank | Hs.118021 | fulllength | 29 | NM_001092; active breakpoint cluster region-related protein isoform b |
| | | | | | NM_021962; active breakpoint cluster region-related protein isoform a |
| 57 | RefSeq | Hs.272100 | fulllength | 29125 | NM_014144; chromosome 11 open reading frame 21 |
| 58 | GenBank | Hs.3989 | fulllength | 23654 | NM_012401; plexin B2 |
| 59 | GenBank | Hs.173179 | | | |
| 60 | GenBank | Hs.10247 | fulllength | 214 | NM_001627; activated leukocyte cell adhesion molecule |
| 61 | GenBank | Hs.49500 | | 23231 | |
| 62 | RefSeq | Hs.271954 | fulllength | 10077 | NM_005705; tumor-suppressing subtransferable candidate 6 isoform 2 |
| | | | | | NM_139022; tumor-suppressing subtransferable candidate 6 isoform 1 |
| | | | | | NM_139023; tumor-suppressing subtransferable candidate 6 isoform 4 |
| | | | | | NM_139024; tumor-suppressing subtransferable candidate 6 isoform 3 |
| 63 | GenBank | Hs.350684 | fulllength | 143098 | NM_173496; hypothetical protein FLJ32798 |
| 64 | GenBank | Hs.39132 | fulllength | 84270 | NM_032310; hypothetical protein MGC11115 |
| 65 | RefSeq | Hs.17936 | fulllength | 25958 | NM_015492; DKFZP434H132 protein |
| 66 | GenBank | Hs.41294 | | | |
| 67 | GenBank | Hs.49500 | | 23231 | |
| 68 | GenBank | Hs.17936 | fulllength | 25958 | NM_015492; DKFZP434H132 protein |
| 69 | GenBank | Hs.301763 | fulllength | 23048 | |
| 70 | RefSeq | Hs.294101 | fulllength | 5090 | NM_006195; pre-B-cell leukemia transcription factor 3 |
| 71 | RefSeq | Hs.18800 | fulllength | 54877 | NM_017742; hypothetical protein FLJ20281 NM_032724; hypothetical protein |
| | | | | | FLJ20281 |
| 72 | RefSeq | Hs.78629 | fulllength | 481 | NM_001677; ATPase, Na+/K+ transporting, beta 1 polypeptide |
| 73 | GenBank | Hs.11607 | fulllength | 157254 | NM_002360; v-maf musculoaponeurotic fibrosarcoma oncogene homolog K |
| | | | | | NM_152561; hypothetical protein FLJ32205 |
| 74 | GenBank | Hs.283844 | fulllength | 81855 | NM_006902; paired mesoderm homeobox 1 isoform pmx-1a NM_022716; paired |
| | | | | | mesoderm homeobox 1 isoform pmx-1b NM_030971; similar to rat tricarboxylate |
| | | | | | carrier-like protein |
| 75 | RefSeq | Hs.294133 | fulllength | 50865 | NM_015987; heme binding protein 1 |
| 76 | RefSeq | Hs.8087 | fulllength | 51754 | NM_016446; NAG-5 protein |
| 77 | GenBank | Hs.184736 | fulllength | 56271 | |
| 78 | RefSeq | Hs.172199 | fulllength | 113 | NM_001114; adenylate cyclase 7 |
| 79 | GenBank | Hs.76364 | fulllength | 199 | NM_001623; allograft inflammatory factor 1 isoform 3 NM_004847; allograft |
| | | | | | inflammatory factor 1 isoform 2 NM_032955; allograft inflammatory factor 1 |
| | | | | | isoform 1 |
| 80 | GenBank | Hs.54673 | fulllength | 8741 | NM_003808; tumor necrosis factor ligand superfamily, member 13 isoform alpha |
| | | | | | precursor NM_172087; tumor necrosis factor ligand superfamily, member 13 |
| | | | | | isoform beta NM_172088; tumor necrosis factor ligand superfamily, member 13 |
| | | | | | isoform gamma NM_172089; tumor necrosis factor ligand superfamily, member |
| | | | | | 13 isoform delta |
| 81 | RefSeq | Hs.283844 | fulllength | 81855 | NM_030971; similar to rat tricarboxylate carrier-like protein |
| 82 | GenBank | Hs.15093 | fulllength | 51523 | NM_016463; hypothetical protein HSPC195 |
| 83 | GenBank | Hs.288862 | fulllength | 8030 | NM_005436; DNA segment on chromosome 10 (unique) 170 |
| 84 | RefSeq | Hs.77637 | fulllength | 3201 | NM_002141; homeobox protein A4 |
| 85 | RefSeq | Hs.86958 | fulllength | 3455 | NM_000874; interferon (alpha, beta and omega) receptor 2 |
| 86 | GenBank | Hs.103512 | fulllength | 124540 | NM_138962; musashi 2 isoform a NM_170721; musashi 2 isoform b |
| 87 | GenBank | Hs.1817 | fulllength | 4353 | NM_000250; myeloperoxidase |
| 88 | RefSeq | Hs.24697 | fulllength | 8418 | XR_000114; |
| 89 | GenBank | Hs.2733 | fulllength | 3212 | NM_002145; homeo box B2 |
| 90 | GenBank | Hs.31551 | fulllength | 862 | NM_004349; acute myelogenous leukemia 1 translocation 1 protein isoform |
| | | | | | MTG8a NM_175634; acute myelogenous leukemia 1 translocation 1 protein |
| | | | | | isoform MTG8b NM_175635; acute myelogenous leukemia 1 translocation 1 |
| | | | | | protein isoform MTG8c NM_175636; acute myelogenous leukemia 1 |
| | | | | | translocation 1 protein isoform MTG8c |
| 91 | GenBank | Hs.286055 | fulllength | 1124 | NM_004067; chimerin (chimaerin) 2 |
| 92 | GenBank | Hs.72639 | est | | |
| 93 | GenBank | Hs.78629 | fulllength | 481 | NM_001677; ATPase, Na+/K+ transporting, beta 1 polypeptide |
| 94 | GenBank | Hs.34656 | est | | |
| 95 | RefSeq | Hs.62 | fulllength | 5782 | NM_002835; protein tyrosine phosphatase, non-receptor type 12 |
| 96 | GenBank | Hs.171625 | fulllength | 84833 | NM_002650; phosphatidylinositol 4-kinase, catalytic, alpha polypeptide isoform 1 |
| | | | | | NM_032747; upregulated during skeletal muscle growth 5 NM_058004; |
| | | | | | phosphatidylinositol 4-kinase, catalytic, alpha polypeptide isoform 2 |
| 97 | RefSeq | Hs.334874 | fulllength | 5297 | NM_002650; phosphatidylinositol 4-kinase, catalytic, alpha polypeptide isoform 1 |
| | | | | | NM_058004; phosphatidylinositol 4-kinase, catalytic, alpha polypeptide isoform 2 |
| 98 | GenBank | Hs.22983 | fulllength | 55757 | NM_020121; UDP-glucose:glycoprotein glucosyltransferase 2 |
| 99 | GenBank | Hs.54673 | fulllength | 8741 | NM_003808; tumor necrosis factor ligand superfamily, member 13 isoform alpha |
| | | | | | precursor NM_172087; tumor necrosis factor ligand superfamily, member 13 |
| | | | | | isoform beta NM_172088; tumor necrosis factor ligand superfamily, member 13 |
| | | | | | isoform gamma NM_172089; tumor necrosis factor ligand superfamily, member |
| | | | | | 13 isoform delta |
| 100 | RefSeq | Hs.211588 | fulllength | 5457 | NM_006237; POU domain, class 4, transcription factor 1 |

TABLE 15

| | affy id | HUGO name | Title | MapLocation | Sequence Type | Go_Biological_Process |
|---|---|---|---|---|---|---|
| 1 | 208268_at | ADAM28 | a disintegrin and | 8p21.1 | Exemplarsequence | “GO: 7283; spermatogenesis; traceable |
| | | | metalloproteinase domain 28 | | | author statement GO: 6508; proteolysis |
| | | | | | | and peptidolysis; inferred from |
| | | | | | | electronic annotation” |
| 2 | 242738_s_at | ATBF1 | Homo sapiens, clone | | Consensussequence | |
| | | | IMAGE: 5288537, mRNA | | | |
| 3 | 202946_s_at | BTBD3 | BTB (POZ) domain containing 3 | 20p12.1 | Exemplarsequence | |
| 4 | 215567_at | C14orf111 | Homo sapiens cDNA FLJ11574 | | Consensussequence | |
| | | | fis, clone HEMBA1003384. | | | |
| 5 | 209831_x_at | DNASE2 | deoxyribonuclease II, lysosomal | 19p13.2 | Exemplarsequence | “GO: 6259; DNA metabolism; traceable |
| | | | | | | author statement GO: 6915; apoptosis; |
| | | | | | | inferred from electronic annotation” |
| 6 | 203187_at | DOCK1 | dedicator of cyto-kinesis 1 | 10q26.13-q26.3 | Exemplarsequence | “GO: 7165; signal transduction; |
| | | | | | | traceable author statement GO: 7229; |
| | | | | | | integrin-mediated signaling pathway; |
| | | | | | | traceable author statement GO: 7264; |
| | | | | | | small GTPase mediated signal |
| | | | | | | transduction; traceable author statement |
| | | | | | | GO: 6915; apoptosis; traceable author |
| | | | | | | statement GO: 6911; phagocytosis, |
| | | | | | | engulfment; traceable author statement” |
| 7 | 208872_s_at | DP1 | likely ortholog of mouse deleted | 5q22-q23 | Consensussequence | |
| | | | in polyposis 1 | | | |
| 8 | 204160_s_at | ENPP4 | ectonucleotide | 6p12.3 | Consensussequence | “GO: 9117; nucleotide metabolism; |
| | | | pyrophosphatase/phosphodiesterase | | | inferred from electronic annotation” |
| | | | 4 (putative function) | | | |
| 9 | 242784_at | ETS2 | ESTs | | Consensussequence | |
| 10 | 219981_x_at | FLJ20813 | hypothetical protein FLJ20813 | 19q13.43 | Exemplarsequence | |
| 11 | 213260_at | FOXC1 | Homo sapiens cDNA FLJ11796 | | Consensussequence | |
| | | | fis, clone HEMBA1006158, highly | | | |
| | | | similar to Homo sapiens | | | |
| | | | transcription factor forkhead-like 7 | | | |
| | | | (FKHL7) gene. | | | |
| 12 | 202967_at | GSTA4 | glutathione S-transferase A4 | 6p12.1 | Exemplarsequence | “GO: 6950; response to stress; |
| | | | | | | not recorded GO: 6803; glutathione |
| | | | | | | conjugation reaction; inferred from |
| | | | | | | electronic annotation” |
| 13 | 214455_at | HIST1H2BC | histone 1, H2bc | 6p21.3 | Consensussequence | “GO: 6334; nucleosome assembly; |
| | | | | | | non-traceable author statement GO: |
| | | | | | | 7001; chromosome organization and |
| | | | | | | biogenesis (sensu Eukarya); inferred |
| | | | | | | from electronic annotation” |
| 14 | 211220_s_at | HSF2 | heat shock transcription factor 2 | 6q22.32 | Exemplarsequence | “GO: 6355; regulation of transcription, |
| | | | | | | DNA-dependent; inferred from |
| | | | | | | electronic annotation GO: 6366; |
| | | | | | | transcription from Pol II |
| | | | | | | promoter; traceable author statement” |
| 15 | 227370_at | KIAA1946 | KIAA1946 protein | 2q32.1 | Consensussequence | |
| 16 | 208767_s_at | LAPTM4B | lysosomal associated protein | 8q22.1 | Consensussequence | |
| | | | transmembrane 4 beta | | | |
| 17 | 214039_s_at | LAPTM4B | lysosomal associated protein | 8q22.1 | Consensussequence | |
| | | | transmembrane 4 beta | | | |
| 18 | 235391_at | LOC137392 | similar to CG6405 gene product | 8q21.3 | Consensussequence | |
| 19 | 217975_at | LOC51186 | pp21 homolog | Xq22.1 | Exemplarsequence | |
| 20 | 208858_s_at | MBC2 | likely ortholog of mouse | 12q13.13 | Exemplarsequence | “GO: 7186; G-protein coupled receptor |
| | | | membrane bound C2 domain | | | protein signaling pathway; inferred |
| | | | containing protein | | | from electronic annotation” |
| 21 | 201620_at | MBTPS1 | membrane-bound transcription | 16q24 | Exemplarsequence | “GO: 6629; lipid metabolism; inferred |
| | | | factor protease, site 1 | | | from electronic annotation GO: |
| | | | | | | 6508; proteolysis and peptidolysis; |
| | | | | | | traceable author statement GO: 8203; |
| | | | | | | cholesterol metabolism; inferred |
| | | | | | | from electronic annotation” |
| 22 | 203948_s_at | MPO | myeloperoxidase | 17q23.1 | Exemplarsequence | “GO: 6916; anti-apoptosis; traceable |
| | | | | | | author statement GO: 6952; defense |
| | | | | | | response; traceable author statement |
| | | | | | | GO: 6979; response to oxidative stress; |
| | | | | | | traceable author statement” |
| 23 | 202600_s_at | NRIP1 | nuclear receptor interacting | 21q11.2 | Consensussequence | “GO: 6355; regulation of transcription, |
| | | | protein 1 | | | DNA-dependent; inferred from |
| | | | | | | electronic annotation GO: 6350; |
| | | | | | | transcription; traceable author |
| | | | | | | statement” |
| 24 | 225864_at | NSE2 | Homo sapiens cDNA FLJ23705 | | Consensussequence | |
| | | | fis, clone HEP11066. | | | |
| 25 | 217848_s_at | PP | pyrophosphatase (inorganic) | 10q11.1-q24 | Exemplarsequence | |
| 26 | 208994_s_at | PPIG | peptidyl-prolyl isomerase G | 2q31.1 | Consensussequence | “GO: 6371; mRNA splicing; traceable |
| | | | (cyclophilin G) | | | author statement GO: 6457; |
| | | | | | | protein folding; inferred from |
| | | | | | | electronic annotation” |
| 27 | 218599_at | REC8L1 | Rec8p, a meiotic recombination | 14q11.2-q12 | Exemplarsequence | “GO: 7126; meiosis; traceable |
| | | | and sister chromatid cohesion | | | author statement GO: 7283; |
| | | | phosphoprotein of the rad21p | | | spermatogenesis; traceable author |
| | | | family | | | statement GO: 7131; meiotic |
| | | | | | | recombination; traceable author |
| | | | | | | statement GO: 7062; sister chromatid |
| | | | | | | cohesion; traceable author statement” |
| 28 | 210365_at | RUNX1 | runt-related transcription factor 1 | 21q22.3 | Exemplarsequence | “GO: 6355; regulation of transcription, |
| | | | (acute myeloid leukemia 1; aml1 | | | DNA-dependent; non-traceable author |
| | | | oncogene) | | | statement GO: 7275; development; |
| | | | | | | traceable author statement GO: |
| | | | | | | 8151; cell growth and/or maintenance; |
| | | | | | | inferred from electronic annotation |
| | | | | | | GO: 7048; oncogenesis; traceable |
| | | | | | | author statement” |
| 29 | 201427_s_at | SEPP1 | selenoprotein P, plasma, 1 | 5q31 | Exemplarsequence | “GO: 6979; response to oxidative stress; |
| | | | | | | traceable author statement” |
| 30 | 226419_s_at | SFRS1 | Homo sapiens cDNA FLJ30048 | | Consensussequence | |
| | | | fis, clone ADRGL1000018. | | | |
| 31 | 203753_at | TCF4 | transcription factor 4 | 18q21.1 | Exemplarsequence | “GO: 6357; regulation of transcription |
| | | | | | | from Pol II promoter; traceable |
| | | | | | | author statement” |
| 32 | 210665_at | TFPI | tissue factor pathway inhibitor | 2q31-q32.1 | Exemplarsequence | “GO: 7596; blood coagulation; |
| | | | (lipoprotein-associated | | | traceable author statement” |
| | | | coagulation inhibitor) | | | |
| 33 | 201688_s_at | TPD52 | tumor protein D52 | 8q21 | Consensussequence | “GO: 7345; embryogenesis and |
| | | | | | | morphogenesis; traceable author |
| | | | | | | statement GO: 7048; oncogenesis; |
| | | | | | | traceable author statement” |
| 34 | 201689_s_at | TPD52 | tumor protein D52 | 8q21 | Consensussequence | “GO: 7345; embryogenesis and |
| | | | | | | morphogenesis; traceable author |
| | | | | | | statement GO: 7048; oncogenesis; |
| | | | | | | traceable author statement” |
| 35 | 201690_s_at | TPD52 | tumor protein D52 | 8q21 | Consensussequence | “GO: 7345; embryogenesis and |
| | | | | | | morphogenesis; traceable author |
| | | | | | | statement GO: 7048; oncogenesis; |
| | | | | | | traceable author statement” |
| 36 | 208762_at | UBL1 | ubiquitin-like 1 (sentrin) | 2q33 | Exemplarsequence | “GO: 6281; DNA repair; |
| | | | | | | traceable author statement” |
| 37 | 33148_at | ZFR | zinc finger RNA binding protein | 5p13.3 | Consensussequence | |
| 38 | 214042_s_at | RPL22 | ribosomal protein L22 | 1p36.3-p36.2 | Consensussequence | “GO: 6412; protein biosynthesis; |
| | | | | | | traceable author statement” |
| 39 | 215447_at | | Homo sapiens mRNA; cDNA | | Consensussequence | |
| | | | DKFZp586J0323 (from clone | | | |
| | | | DKFZp586J0323) | | | |
| 40 | 222380_s_at | | ESTs | | Consensussequence | |
| 41 | 225547_at | | Homo sapiens cDNA FLJ39478 | | Consensussequence | |
| | | | fis, clone PROST2013605. | | | |
| 42 | 230620_at | | ESTs | | Consensussequence | |

| | | | Transcript |
|---|---|---|---|
| | Go_Cellular_Component | Go_Molecular_Function | ID |
| 1 | “GO: 16021; integral to membrane; inferred from | “GO: 4222; metalloendopeptidase activity; inferred from electronic annotation | Hs.174030.1 |
| | electronic annotation” | GO: 8270; zinc ion binding; inferred from electronic annotation GO: 16787; | |
| | | hydrolase activity; inferred from electronic | |
| | | annotation” | |
| 2 | | | Hs.163208.0 |
| 3 | | “GO: 5515; protein binding; inferred from electronic annotation” | Hs.7935.0 |
| 4 | | | Hs.287426.0 |
| 5 | “GO: 5764; lysosome; traceable author statement” | “GO: 16787; hydrolase activity; inferred from electronic | Hs.118243.0 |
| | | annotation GO: 3677; DNA binding; traceable author statement GO: 4519; | |
| | | endonuclease activity; inferred from electronic annotation GO: | |
| | | 4531; deoxyribonuclease II activity; traceable author statement” | |
| 6 | “GO: 5737; cytoplasm; traceable author | “GO: 5524; ATP binding; inferred from electronic annotation GO: | Hs.82295.0 |
| | statement” | 5096; GTPase activator activity; traceable author statement” | |
| 7 | “GO: 16021; integral to membrane; non-traceable | | Hs.178112.0 |
| | author statement” | | |
| 8 | | “GO: 16787; hydrolase activity; inferred from electronic annotation” | Hs.54037.0 |
| 9 | | | Hs.213021.0 |
| 10 | | | Hs.306203.0 |
| 11 | | | Hs.284186.0 |
| 12 | | “GO: 4364; glutathione transferase activity; traceable author statement GO: | Hs.169907.0 |
| | | 16740; transferase activity; inferred from electronic annotation” | |
| 13 | “GO: 5634; nucleus; inferred from electronic | “GO: 3677; DNA binding; non-traceable author statement” | Hs.239884.0 |
| | annotation GO: 786; nucleosome; non-traceable | | |
| | author statement GO: 5694; chromosome; inferred | | |
| | from electronic annotation” | | |
| 14 | “GO: 5634; nucleus; inferred from electronic | “GO: 3713; transcription co-activator activity; traceable author statement GO: | Hs.158195.1 |
| | annotation” | 3773; heat shock protein activity; inferred from electronic annotation GO: 3700; | |
| | | transcription factor activity; traceable author statement” | |
| 15 | | | Hs.25329.0 |
| 16 | “GO: 16021; integral to membrane; inferred from | | Hs.296398.0 |
| | electronic annotation” | | |
| 17 | “GO: 16021; integral to membrane; inferred from | | Hs.296398.1 |
| | electronic annotation” | | |
| 18 | | | Hs.87672.0 |
| 19 | | | Hs.15984.0 |
| 20 | “GO: 16021; integral to membrane; inferred from | “GO: 1584; rhodopsin-like receptor activity; inferred from electronic annotation” | Hs.8309.0 |
| | electronic annotation” | | |
| 21 | “GO: 5788; endoplasmic reticulum lumen; | “GO: 8233; peptidase activity; inferred from electronic annotation GO: | Hs.75890.0 |
| | traceable author statement GO: 5794; Golgi | 4289; subtilase activity; inferred from electronic annotation” | |
| | apparatus; inferred from electronic annotation | | |
| | GO: 16021; integral to membrane; inferred from | | |
| | electronic annotation” | | |
| 22 | “GO: 5764; lysosome; traceable author statement | “GO: 4601; An_peroxidase; peroxidase activity; 6.4e−161; extended: inferred | Hs.1817.0 |
| | GO: 5634; nucleus; traceable author statement” | from electronic annotation GO: 3682; chromatin binding; traceable author | |
| | | statement GO: 16687; myeloperoxidase activity; inferred from electronic | |
| | | annotation GO: 16685; eosinophil peroxidase activity; inferred from | |
| | | electronic annotation GO: 5509; calcium ion binding; inferred from electronic | |
| | | annotation GO: 16491; oxidoreductase activity; inferred from electronic | |
| | | annotation GO: 16686; lactoperoxidase activity; inferred from electronic | |
| | | annotation” | |
| 23 | “GO: 5634; nucleus; traceable author statement” | “GO: 3713; transcription co-activator activity; traceable author statement” | Hs.155017.0 |
| 24 | | | Hs.49136.0 |
| 25 | | “GO: 4427; 3.6.1.1; inorganic diphosphatase activity; 4.18e−116; extended: | Hs.184011.0 |
| | | inferred from electronic annotation GO: 16462; Pyrophosphatase; | |
| | | pyrophosphatase activity; 4.4e−129; extended: Unknown” | |
| 26 | “GO: 5654; nucleoplasm; traceable author | “GO: 16853; isomerase activity; inferred from electronic annotation GO: 30051; | Hs.77965.0 |
| | statement” | FK506-sensitive peptidyl-prolyl cis-trans isomerase; inferred from electronic | |
| | | annotation GO: 4600; cyclophilin; traceable author statement GO: 8248; | |
| | | pre-mRNA splicing factor activity; traceable author statement GO: 42027; | |
| | | cyclophilin-type peptidy-prolyl cis-trans isomerase activity; inferred from | |
| | | electronic annotation” | |
| 27 | “GO: 5634; nucleus; traceable author statement” | | Hs.4767.0 |
| 28 | “GO: 5634; nucleus; non-traceable author | “GO: 3700; transcription factor activity; traceable author statement GO: 5524; | Hs.129914.4 |
| | statement” | ATP binding; non-traceable author statement GO: 3677; Runt; DNA | |
| | | binding activity; 1.2e−102; extended: Unknown” | |
| 29 | | “GO: 8430; selenium binding; traceable author statement” | Hs.3314.0 |
| 30 | | | Hs.238956.1 |
| 31 | “GO: 5634; nucleus; traceable author statement” | “GO: 3677; DNA binding; inferred from electronic annotation GO: 3702; RNA | Hs.326198.0 |
| | | polymerase II transcription factor activity; traceable author statement” | |
| 32 | | “GO: 4867; serine protease inhibitor activity; inferred from electronic annotation | Hs.170279.1 |
| | | GO: 5209; plasma protein; not recorded GO: 5211; plasma | |
| | | glycoprotein; not recorded” | |
| 33 | “GO: 5871; kinasin complex; inferred from | | Hs.2384.0 |
| | electronic annotation” | | |
| 34 | “GO: 5871; kinesin complex; inferred from | | Hs.2384.0 |
| | electronic annotation” | | |
| 35 | “GO: 5871; kinasin complax; inferred from | | Hs.2384.0 |
| | electronic annotation” | | |
| 36 | “GO: 5634; nucleus; traceable author statement” | “GO: 4840; ubiquitin conjugating enzyme activity; traceable author statement” | Hs.81424.0 |
| | GO: 5643; nuclear pore; traceable author | | |
| | statement” | | |
| 37 | “GO: 5634; nucleus; inferred from electronic | “GO: 3723; RNA binding; inferred from electronic annotation” | 5 |
| | annotation” | | |
| 38 | “GO: 5840; ribosome; inferred from electronic | “GO: 3723; RNA binding; traceable author statement GO: 8201; heparin binding; | Hs.326249.0 |
| | annotation GO: 5842; cytosolic large ribosomal | inferred from electronic annotation GO: 3735; structural constituent of | |
| | subunit (sensu Eukarya); traceable author | ribosome; traceable author statement” | |
| | statement GO: 5622; intracellular; inferred from | | |
| | electronic annotation” | | |
| 39 | | | Hs.102301.0 |
| 40 | | | Hs.124620.0 |
| 41 | | | Hs.292815.0 |
| 42 | | | Hs.143587.0 |

| | Sequence | | Sequence | | | | |
|---|---|---|---|---|---|---|---|
| | Derived From | Sequence ID | Source | Unigene_Accession | Cluster_Type | LocusLink | Full_Length_Reference_Seq |
| 1 | NM_021777.1 | g11496993 | RefSeq | Hs.174030 | fulllength | 10863 | NM_014265; a disintegrin and |
| | | | | | | | metalloproteinase domain 28 isoform 1 |
| | | | | | | | preproprotein NM_021777; a |
| | | | | | | | disintegrin and metalloproteinase |
| | | | | | | | domain 28 isoform 3 preproprotein |
| | | | | | | | NM_021778; a disintegrin and |
| | | | | | | | metalloproteinase domain 28 isoform 2 |
| | | | | | | | preproprotein |
| 2 | BG402859 | Hs.163208.0.A1 | GenBank | Hs.108806 | | | |
| 3 | NM_014962.1 | g7662401 | RefSeq | Hs.7935 | fulllength | 22903 | NM_014982; BTB/POZ domain containing |
| | | | | | | | protein 3 isoform a NM_181443; BTB/POZ |
| | | | | | | | domain containing protein 3 isoform b |
| 4 | AU144919 | Hs.287426.0 | GenBank | Hs.287426 | | | |
| 5 | AB004574.1 | g3184394 | GenBank | Hs.118243 | fulllength | 1777 | NM_001375; deoxyribonuclease II, lysosomal |
| 6 | NM_001380.1 | g4503354 | RefSeq | Hs.82295 | fulllength | 1793 | NM_001380; dedicator of cyto-kinesis 1 |
| 7 | AA814140 | Hs.178112.0.S1 | GenBank | Hs.178112 | fulllength | 7905 | NM_005669; likely ortholog of mouse |
| | | | | | | | deleted in polyposis 1 |
| 8 | AW194947 | Hs.54037.0 | GenBank | Hs.54037 | fulllength | 22875 | NM_014936; ectonucleotide pyrophosphatase/ |
| | | | | | | | phosphodiesterase 4 (putative function) |
| 9 | AV646177 | Hs.213021.0.A1 | GenBank | Hs.213021 | est | | |
| 10 | NM_017961.1 | g8923685 | RefSeq | Hs.288995 | fulllength | 55044 | NM_017961; hypothetical protein FLJ20813 |
| 11 | AU145890 | Hs.284186.0.A2 | GenBank | Hs.284186 | | | |
| 12 | NM_001512.1 | g4504172 | RefSeq | Hs.169907 | fulllength | 2941 | NM_001512; glutathione S-transferase A4 |
| 13 | NM_003526.1 | Hs.239884.0.S1 | GenBank | Hs.356901 | fulllength | 8347 | NM_003526; H2B histone family, member L |
| 14 | BC005329.1 | g13529106 | GenBank | Hs.158195 | fulllength | 3298 | NM_004506; heat shock transcription factor 2 |
| 15 | AW043602 | Hs.25329.0.A1 | GenBank | Hs.172792 | fulllength | 165215 | NM_177454; KIAA1946 protein |
| 16 | AW149681 | Hs.296398.0.A1 | GenBank | Hs.296398 | fulllength | 55353 | NM_018407; |
| | | | | | | | lysosomal-associated transmembrane protein |
| | | | | | | | 4 beta |
| 17 | T15777 | Hs.296398.1.A1 | GenBank | Hs.296398 | fulllength | 55353 | NM_018407; lysosomal-associated |
| | | | | | | | transmembrane protein 4 beta |
| 18 | AW960748 | Hs.87672.0_RC | GenBank | Hs.403869 | fulllength | 137392 | NM_145269; similar to CG6405 gene product |
| 19 | NM_016303.1 | g10047099 | RefSeq | Hs.15984 | fulllength | 51186 | NM_016303; pp21 homolog |
| 20 | BC004998.1 | g13436457 | GenBank | Hs.8309 | fulllength | 23344 | NM_015292; KIAA0747 protein |
| 21 | NM_003791.1 | g4506774 | RefSeq | Hs.75890 | fulllength | 8720 | NM_003791; site-1 protease preproprotein |
| 22 | J02694.1 | g189039 | GenBank | Hs.1817 | fulllength | 4353 | NM_000250; myeloperoxidase |
| 23 | AI824012 | Hs.155017.0.S1 | GenBank | Hs.155017 | fulllength | 8204 | NM_003489; receptor interacting protein 140 |
| 24 | AL039862 | Hs.49136.0.A1 | GenBank | Hs.49136 | | | |
| 25 | NM_021129.1 | g11056043 | RefSeq | Hs.184011 | fulllength | 5464 | NM_021129; inorganic pyrophosphatase |
| 26 | NM_004792.1 | Hs.77965.0_RC | GenBank | Hs.77965 | fulllength | 9360 | NM_004792; peptidyl-prolyl isomerase G |
| | | | | | | | (cyclophilin G) |
| 27 | NM_005132.1 | g9845292 | RefSeq | Hs.4767 | fulllength | 9985 | NM_005132; Rec8p, a meiotic recombination |
| | | | | | | | and sister chromatid cohesion pho |
| 28 | D43967.1 | g966994 | GenBank | Hs.129914 | fulllength | 861 | NM_001754; runt-related transcription factor 1 |
| | | | | | | | (acute myeloid leukemia 1; aml1 oncogene) |
| 29 | NM_005410.1 | g4885590 | RefSeq | Hs.275775 | fulllength | 6414 | NM_005410; selenoprotein P precursor |
| 30 | AA046439 | Hs.238956.1.A1 | GenBank | Hs.238956 | | | |
| 31 | NM_003199.1 | g4507398 | RefSeq | Hs.326198 | fulllength | 6925 | NM_003199; transcription factor 4 isoform b |
| 32 | AF021834.1 | g4103170 | GenBank | Hs.170279 | fulllength | 7035 | NM_006287; tissue factor pathway inhibitor |
| | | | | | | | (lipoprotein-associated coagulation inhibitor) |
| 33 | BE974098 | Hs.2384.0.S2 | GenBank | Hs.2384 | fulllength | 7163 | NM_005079; tumor protein D52 |
| 34 | BE974098 | Hs.2384.0.S2 | GenBank | Hs.2384 | fulllength | 7163 | NM_005079; tumor protein D52 |
| 35 | BE974098 | Hs.2384.0.S2 | GenBank | Hs.2384 | fulllength | 7163 | NM_005079; tumor protein D52 |
| 36 | U83117.1 | g1769601 | GenBank | Hs.81424 | fulllength | 7341 | NM_003352; ubiquitin-like 1 (sentrin) |
| 37 | AI459274 | 4923288_rc | GenBank | Hs.173518 | fulllength | 51663 | NM_016107; M-phase phosphoprotein |
| | | | | | | | homolog |
| 38 | AW071997 | Hs.326249.0.A1 | GenBank | Hs.326249 | fulllength | 6146 | NM_000983; ribosomal protein L22 proprotein |
| 39 | AL080215.1 | Hs.102301.0 | GenBank | Hs.102301 | | | |
| 40 | AI907083 | Hs.124620.0_RC | GenBank | Hs.124620 | est | | |
| 41 | BG169443 | Hs.292815.0.A1 | GenBank | Hs.372680 | | | |
| 42 | BE550967 | Hs.143587.0.A1 | GenBank | Hs.143587 | est | | |

TABLE 16

| | affy id | HUGO name | fc | p | q | stn | t | Title | MapLocation |
|---|---|---|---|---|---|---|---|---|---|
| 1 | 201691_s_at | TPD52 | −2.11 | 3.69e−08 | 1.32e−03 | −0.35 | −5.70 | tumor protein D52 | 8q21 |
| 2 | 213217_at | ADCY2 | −3.18 | 8.65e−08 | 1.55e−03 | −0.34 | −5.52 | adenylate cyclase 2 (brain) | 5p15.3 |
| 3 | 210487_at | DNTT | −5.47 | 1.75e−07 | 2.08e−03 | −0.34 | −5.39 | deoxynucleotidyltransferase, terminal | 10q23-q24 |
| 4 | 201690_s_at | TPD52 | −1.87 | 3.04e−07 | 2.72e−03 | −0.32 | −5.26 | tumor protein D52 | 8q21 |
| 5 | 225547_at | | −1.17 | 7.43e−07 | 3.90e−03 | −0.36 | −5.23 | Homo sapiens cDNA FLJ39478 fis, clone | |
| | | | | | | | | PROST2013605. | |
| 6 | 210665_at | TFPI | −2.17 | 5.79e−07 | 3.90e−03 | −0.33 | −5.17 | tissue factor pathway inhibitor | 2q31-q32.1 |
| | | | | | | | | (lipoprotein-associated coagulation | |
| | | | | | | | | inhibitor) | |
| 7 | 227370_at | KIAA1946 | −2.22 | 7.62e−07 | 3.90e−03 | −0.31 | −5.07 | KIAA1946 protein | 2q32.1 |
| 8 | 235721_at | | −1.87 | 9.11e−07 | 4.08e−03 | −0.31 | −5.03 | Homo sapiens cDNA FLJ37066 fis, clone | |
| | | | | | | | | BRACE2015132, weakly similar to | |
| | | | | | | | | Drosophila melanogaster Oregon R | |
| | | | | | | | | cytoplasmic basic protein (deltex) mRNA. | |
| 9 | 224150_s_at | BITE | −1.49 | 3.32e−06 | 1.32e−02 | −0.29 | −4.75 | p10-binding protein | 3q22-q23 |
| 10 | 224473_x_at | KIAA1813 | −1.24 | 6.36e−06 | 1.65e−02 | −0.32 | −4.71 | KIAA1813 protein | 10q24 |
| 11 | 244611_at | | −1.58 | 5.33e−06 | 1.65e−02 | −0.30 | −4.69 | ESTs, Highly similar to thyroid hormone | |
| | | | | | | | | receptor-associated protein, 240 kDa | |
| | | | | | | | | subunit [Homo sapiens] [H. sapiens] | |
| 12 | 201689_s_at | TPD52 | −1.89 | 4.74e−06 | 1.65e−02 | −0.29 | −4.68 | tumor protein D52 | 8q21 |
| 13 | 220022_at | ZNF334 | −1.82 | 5.54e−06 | 1.65e−02 | −0.29 | −4.66 | zinc finger protein 334 | 20q13.12 |
| 14 | 215567_at | | −1.33 | 6.81e−06 | 1.65e−02 | −0.30 | −4.65 | Homo sapiens cDNA FLJ11574 fis, clone | |
| | | | | | | | | HEMBA1003384. | |
| 15 | 225864_at | | −1.64 | 7.38e−06 | 1.65e−02 | −0.29 | −4.61 | Homo sapiens cDNA FLJ23705 fis, clone | |
| | | | | | | | | HEP11066. | |
| 16 | 232081_at | | −2.33 | 7.36e−06 | 1.65e−02 | −0.28 | −4.58 | Homo sapiens EST from clone 208499, | |
| | | | | | | | | full insert | |
| 17 | 220602_s_at | FLJ22795 | −1.64 | 1.09e−05 | 2.29e−02 | −0.30 | −4.56 | hypothetical protein FLJ22795 | 15q24.3 |
| 18 | 214373_at | PPP4R2 | −1.28 | 1.17e−05 | 2.32e−02 | −0.28 | −4.49 | protein phosphatase 4, regulatory | 3q29 |
| | | | | | | | | subunit 2 | |
| 19 | 211220_s_at | HSF2 | −1.28 | 1.37e−05 | 2.42e−02 | −0.29 | −4.48 | heat shock transcription factor 2 | 6q22.32 |
| 20 | 212385_at | | −1.71 | 1.34e−05 | 2.42e−02 | −0.28 | −4.45 | Homo sapiens cDNA FLJ11918 fis, clone | |
| | | | | | | | | HEMBB1000272. | |
| 21 | 208268_at | ADAM28 | −1.52 | 1.49e−05 | 2.42e−02 | −0.28 | −4.43 | a disintegrin and metalloproteinase | 8p21.1 |
| | | | | | | | | domain 28 | |
| 22 | 228701_at | MGC33510 | −1.57 | 1.43e−05 | 2.42e−02 | −0.27 | −4.42 | hypothetical protein MGC33510 | 8q12.3 |
| 23 | 219981_x_at | FLJ20813 | −1.24 | 2.13e−05 | 2.63e−02 | −0.30 | −4.41 | hypothetical protein FLJ20813 | 19q13.43 |
| 24 | 237311_at | | −1.77 | 1.81e−05 | 2.61e−02 | −0.28 | −4.39 | ESTs | |
| 25 | 230620_at | | −1.31 | 1.89e−05 | 2.61e−02 | −0.28 | −4.39 | ESTs | |
| 26 | 209763_at | NRLN1 | −1.92 | 1.69e−05 | 2.61e−02 | −0.27 | −4.38 | likely ortholog of mouse neuralin 1 | Xq22.3 |
| 27 | 223629_at | PCDHB5 | −1.72 | 1.89e−05 | 2.61e−02 | −0.27 | −4.36 | protocadherin beta 5 | 5q31 |
| 28 | 239175_at | | −1.74 | 2.08e−05 | 2.63e−02 | −0.27 | −4.35 | ESTs | |
| 29 | 233475_at | SNCAIP | −1.55 | 2.01e−05 | 2.63e−02 | −0.27 | −4.35 | synuclein, alpha interacting protein | 5q23.1-q23.3 |
| | | | | | | | | (synphilin) | |
| 30 | 202946_s_at | BTBD3 | −1.38 | 3.00e−05 | 2.85e−02 | −0.29 | −4.32 | BTB (POZ) domain containing 3 | 20p12.1 |
| 31 | 215447_at | | −1.38 | 2.45e−05 | 2.85e−02 | −0.27 | −4.32 | Homo sapiens mRNA; cDNA | |
| | | | | | | | | DKFZp586J0323 (from clone | |
| | | | | | | | | DKFZp586J0323) | |
| 32 | 203753_at | TCF4 | −1.59 | 2.87e−05 | 2.85e−02 | −0.28 | −4.31 | transcription factor 4 | 18q21.1 |
| 33 | 203705_s_at | FZD7 | −1.35 | 2.82e−05 | 2.85e−02 | −0.27 | −4.30 | frizzled homolog 7 (Drosophila) | 2q33 |
| 34 | 209831_x_at | DNASE2 | 12055 | 4.53e−05 | 3.04e−02 | 0.32 | 46844 | deoxyribonuclease II, lysosomal | 19p13.2 |
| 35 | 218599_at | REC8 | −1.34 | 3.06e−05 | 2.85e−02 | −0.27 | −4.28 | Rec8p, a meiotic recombination and sister | 14q11.2-q12 |
| | | | | | | | | chromatid cohesion phosphoprotein of the | |
| | | | | | | | | rad21p family | |
| 36 | 210365_at | RUNX1 | −1.51 | 3.10e−05 | 2.85e−02 | −0.27 | −4.27 | runt-related transcription factor 1 (acute | 21q22.3 |
| | | | | | | | | myeloid leukemia 1; aml1 oncogene) | |
| 37 | 230392_at | | −1.45 | 2.77e−05 | 2.85e−02 | −0.26 | −4.27 | Homo sapiens cDNA FLJ31096 fis, clone | |
| | | | | | | | | IMR321000207. | |
| 38 | 229620_at | SEPP1 | −1.88 | 2.93e−05 | 2.85e−02 | −0.26 | −4.26 | selenoprotein P, plasma, 1 | 5q31 |
| 39 | 222380_s_at | | −1.31 | 3.34e−05 | 2.85e−02 | −0.27 | −4.26 | ESTs | |
| 40 | 239123_at | | −1.52 | 3.11e−05 | 2.85e−02 | −0.27 | −4.25 | ESTs | |
| 41 | 222186_at | | −1.34 | 3.32e−05 | 2.85e−02 | −0.27 | −4.24 | Homo sapiens mRNA full length insert | |
| | | | | | | | | cDNA clone EUROIMAGE 27080. | |
| 42 | 228840_at | AMOTL1 | −1.93 | 3.16e−05 | 2.85e−02 | −0.26 | −4.24 | angiomotin like 1 | 11q14.3 |
| 43 | 202967_at | GSTA4 | −1.28 | 3.42e−05 | 2.85e−02 | −0.26 | −4.23 | glutathione S-transferase A4 | 6p12.1 |
| 44 | 214378_at | TFPI | −1.33 | 4.19e−05 | 3.03e−02 | −0.28 | −4.22 | tissue factor pathway inhibitor | |
| | | | | | | | | (lipoprotein-associated coagulation | 2q31-q32.1 |
| | | | | | | | | inhibitor) | |
| 45 | 238154_at | | −1.45 | 3.32e−05 | 2.85e−02 | −0.26 | −4.22 | ESTs, Highly simitar to p10-binding | |
| | | | | | | | | protein [Homo sapiens] | |
| | | | | | | | | [H. sapiens] | |
| 46 | 214930_at | KIAA0918 | −3.69 | 3.58e−05 | 2.91e−02 | −0.27 | −4.22 | KIAA0918 protein | 13q31.1 |
| 47 | 237371_at | | −1.93 | 3.79e−05 | 3.02e−02 | −0.26 | −4.21 | ESTs | |
| 48 | 242549_at | | −1.65 | 4.23e−05 | 3.03e−02 | −0.27 | −4.20 | ESTs, Highly similar to KPCN_HUMAN | |
| | | | | | | | | Protein kinase C, nu type (nPKC-nu) | |
| | | | | | | | | (Protein kinase EPK2) [H. sapiens] | |
| 49 | 244537_at | | −1.52 | 3.96e−05 | 3.03e−02 | −0.26 | −4.20 | ESTs | |
| 50 | 236653_at | | −1.52 | 4.16e−05 | 3.03e−02 | −0.26 | −4.18 | ESTs | |
| 51 | 244274_at | | −1.41 | 4.73e−05 | 3.04e−02 | −0.27 | −4.18 | Homo sapiens, clone IMAGE: 5269446, | |
| | | | | | | | | mRNA | |
| 52 | 204846_at | CP | −1.58 | 4.21e−05 | 3.03e−02 | −0.25 | −4.17 | ceruloplasmin (ferroxidase) | 3q23-q25 |
| 53 | 201427_s_at | SEPP1 | −1.88 | 5.03e−05 | 3.12e−02 | −0.27 | −4.16 | selenoprotein P, plasma, 1 | 5q31 |
| 54 | 227803_at | ENPP5 | −1.59 | 4.70e−05 | 3.04e−02 | −0.26 | −4.16 | ectonucleotide pyrophosphatase/ | 6p21.1-p11.2 |
| | | | | | | | | phosphodiesterase 5 (putative function) | |
| 55 | 204430_s_at | SLC2A5 | −1.68 | 4.40e−05 | 3.03e−02 | −0.25 | −4.16 | solute carrier family 2 (facilitated glucose/ | 1p36.2 |
| | | | | | | | | fructose transporter), member 5 | |
| 56 | 220145_at | FLJ21159 | −2.43 | 4.38e−05 | 3.03e−02 | −0.25 | −4.16 | hypothetical protein FLJ21159 | 4q31.3 |
| 57 | 228919_at | | −1.59 | 4.75e−05 | 3.04e−02 | −0.26 | −4.15 | ESTs, Highly similar to cell division cycle | |
| | | | | | | | | 2-like 1, isoform 1; Cell division cycle | |
| | | | | | | | | 2-like 1; PITSLRE protein kinase alpha; | |
| | | | | | | | | p58/GTA protein kinase; | |
| | | | | | | | | galactosyltransferase associated protein | |
| | | | | | | | | kinase; CDC-related protein kinase p58; | |
| | | | | | | | | PITSLRE B [Homo sapiens] [H. sapiens] | |
| 58 | 210815_s_at | CALCRL | −1.54 | 5.12e−05 | 3.12e−02 | −0.26 | −4.14 | calcitonin receptor-like | 2q32.2 |
| 59 | 236363_at | LOC285378 | −1.26 | 6.18e−05 | 3.46e−02 | −0.28 | −4.13 | hypothetical protein LOC285378 | 3p25.1 |
| 60 | 221973_at | LOC150759 | −1.37 | 6.15e−05 | 3.46e−02 | −0.27 | −4.13 | hypothetical protein LOC150759 | 2q11.1 |
| 61 | 244579_at | | −2.32 | 5.14e−05 | 3.12e−02 | −0.26 | −4.13 | ESTs | |
| 62 | 243010_at | MSI2 | −1.50 | 5.81e−05 | 3.46e−02 | −0.26 | −4.12 | musashi homolog 2 (Drosophila) | 17q23.1 |
| 63 | 223800_s_at | LOC96626 | −1.27 | 6.18e−05 | 3.46e−02 | −0.27 | −4.11 | pinch-2 | 2q14-q21 |
| 64 | 213459_at | RPL37A | −1.22 | 6.55e−05 | 3.53e−02 | −0.27 | −4.11 | ribosomal protein L37a | 2q35 |
| 65 | 220377_at | C14orf110 | −2.40 | 6.06e−05 | 3.46e−02 | −0.26 | −4.11 | chromosome 14 open reading frame 110 | 14q32.33 |
| 66 | 235274_at | | −1.35 | 6.75e−05 | 3.53e−02 | −0.26 | −4.09 | ESTs, Weakly similar to hypothetical | |
| | | | | | | | | protein FLJ20489 [Homo sapiens] | |
| | | | | | | | | [H. sapiens] | |
| 67 | 47560_at | FLJ11939 | −1.44 | 6.65e−05 | 3.53e−02 | −0.26 | −4.08 | hypothetical protein FLJ11939 | 19p13.12 |
| 68 | 226586_at | FLJ36928 | −1.39 | 7.66e−05 | 3.72e−02 | −0.27 | −4.06 | hypothetical protein FLJ36928 | 9q22.33 |
| 69 | 234996_at | CALCRL | −1.71 | 6.79e−05 | 3.53e−02 | −0.25 | −4.06 | calcitonin receptor-like | 2q32.2 |
| 70 | 232653_at | | −1.83 | 6.57e−05 | 3.53e−02 | −0.25 | −4.06 | Homo sapiens cDNA FLJ14044 fis, clone | |
| | | | | | | | | HEMBA1006124 | |
| 71 | 221207_s_at | NBEA | −1.39 | 7.00e−05 | 3.58e−02 | −0.26 | −4.06 | neurobeachin | 13q13 |
| 72 | 244286_at | | −1.56 | 7.31e−05 | 3.64e−02 | −0.26 | −4.06 | ESTs, Moderately similar to hypothetical | |
| | | | | | | | | protein FLJ20378 [Homo sapiens] | |
| | | | | | | | | [H. sapiens] | |
| 73 | 202600_s_at | NRIP1 | −1.52 | 7.68e−05 | 3.72e−02 | −0.26 | −4.05 | nuclear receptor interacting protein 1 | 21q11.2 |
| 74 | 231669_at | SEPP1 | −1.62 | 7.33e−05 | 3.64e−02 | −0.26 | −4.05 | selenoprotein P, plasma, 1 | 5q31 |
| 75 | 217755_at | HN1 | 46753 | 9.18e−05 | 4.11e−02 | 0.28 | 38111 | hematological and neurological | 17q25.2 |
| | | | | | | | | expressed 1 | |
| 76 | 222999_s_at | CCNL2 | −1.21 | 8.22e−05 | 3.77e−02 | −0.26 | −4.04 | cyclin L2 | 1p36.33 |
| 77 | 215786_at | | −1.28 | 8.05e−05 | 3.77e−02 | −0.26 | −4.03 | Homo sapiens cDNA FLJ12108 fis, clone | |
| | | | | | | | | MAMMA1000009. | |
| 78 | 243579_at | MSI2 | −1.73 | 8.09e−05 | 3.77e−02 | −0.26 | −4.03 | musashi homolog 2 (Drosophila) | 17q23.1 |
| 79 | 209838_at | TRIP15 | −1.31 | 8.32e−05 | 3.77e−02 | −0.26 | −4.03 | thyroid receptor interacting protein 15 | 15q21.2 |
| 80 | 203826_s_at | PITPNM | 12785 | 1.11e−04 | 4.12e−02 | 0.29 | 38021 | phosphatidylinositol transfer protein, | 11q13 |
| | | | | | | | | membrane-associated | |
| 81 | 243768_at | | −1.27 | 8.06e−05 | 3.77e−02 | −0.25 | −4.02 | ESTs, Weakly similar to hypothetical | |
| | | | | | | | | protein FLJ20489 [Homo sapiens] | |
| | | | | | | | | [H. sapiens] | |
| 82 | 226043_at | AGS3 | −1.31 | 9.81e−05 | 4.12e−02 | −0.26 | −4.00 | activator of G-protein signaling 3 | 9q34.3 |
| 83 | 205251_at | PER2 | −1.32 | 9.39e−05 | 4.11e−02 | −0.26 | −4.00 | period homolog 2 (Drosophila) | 2q37.3 |
| 84 | 208762_at | UBL1 | −1.31 | 9.52e−05 | 4.11e−02 | −0.26 | −3.99 | ubiquitin-like 1 (sentrin) | 2q33 |
| 85 | 226545_at | | −2.14 | 9.48e−05 | 4.11e−02 | −0.25 | −3.99 | Homo sapiens mRNA; cDNA | |
| | | | | | | | | DKFZp586E1624 (from clone | |
| | | | | | | | | DKFZp586E1624) | |
| 86 | 240824_at | | −1.29 | 1.06e−04 | 4.12e−02 | −0.26 | −3.98 | ESTs | |
| 87 | 244740_at | | −1.76 | 9.65e−05 | 4.11e−02 | −0.25 | −3.98 | Homo sapiens, clone MGC: 9913 IMAGE: | |
| | | | | | | | | 3870821, mRNA, complete cds | |
| 88 | 210758_at | PSIP1 | −1.24 | 1.09e−04 | 4.12e−02 | −0.26 | −3.97 | PC4 and SFRS1 interacting protein 1 | 9p22.2 |
| 89 | 233431_x_at | | −1.35 | 1.04e−04 | 4.12e−02 | −0.25 | −3.96 | Homo sapiens cDNA FLJ12393 fis, clone | |
| | | | | | | | | MAMMA1002711. | |
| 90 | 204160_s_at | ENPP4 | −1.60 | 1.06e−04 | 4.12e−02 | −0.25 | −3.95 | ectonucleotide pyrophosphatase/ | 6p12.3 |
| | | | | | | | | phosphodiesterase 4 (putative function) | |
| 91 | 226419_s_at | | −1.37 | 1.10e−04 | 4.12e−02 | −0.25 | −3.95 | Homo sapiens cDNA FLJ30048 fis, clone | |
| | | | | | | | | ADRGL1000018. | |
| 92 | 219934_s_at | STE | −1.53 | 1.17e−04 | 4.24e−02 | −0.26 | −3.95 | sulfotransferase, estrogen-preferring | 4q13.1 |
| 93 | 227202_at | CNTN1 | −2.01 | 9.98e−05 | 4.12e−02 | −0.24 | −3.95 | contactin 1 | 12q11-q12 |
| 94 | 212067_s_at | C1R | −1.35 | 1.12e−04 | 4.12e−02 | −0.25 | −3.95 | complement component 1, | 12p13 |
| | | | | | | | | r subcomponent | |
| 95 | 214043_at | | −1.77 | 1.05e−04 | 4.12e−02 | −0.24 | −3.94 | Homo sapiens mRNA; cDNA | |
| | | | | | | | | DKFZp564P116 (from clone | |
| | | | | | | | | DKFZp564P116) | |
| 96 | 214321_at | NOV | −2.31 | 1.07e−04 | 4.12e−02 | −0.24 | −3.94 | nephroblastoma overexpressed gene | 8q24.1 |
| 97 | 212928_at | KIAA0721 | −1.22 | 1.36e−04 | 4.78e−02 | −0.27 | −3.94 | KIAA0721 protein | 6q22.2 |
| 98 | 242064_at | | −2.01 | 1.05e−04 | 4.12e−02 | −0.24 | −3.94 | Homo sapiens cDNA FLJ90513 fis, clone | |
| | | | | | | | | NT2RP3004355. | |
| 99 | 227798_at | | −1.89 | 1.10e−04 | 4.12e−02 | −0.24 | −3.93 | ESTs | |
| 100 | 239669_at | HIST1H3D | −2.09 | 1.17e−04 | 4.24e−02 | −0.24 | −3.91 | histone 1, H3d | 6p21.3 |

| | Sequence Type | Go_Biological_Process | Go_Cellular_Component |
|---|---|---|---|
| 1 | Exemplarsequence | “GO: 7345; embryogenesis and morphogenesis; traceable | “GO: 5871; kinesin complex; inferred from electronic |
| | | author statement GO: 7048; oncogenesis; traceable author | annotation” |
| | | statement” | |
| 2 | Consensussequence | “GO: 6171; cAMP biosynthesis; non-traceable author statement | “GO: 16021; integral to membrane; non-traceable author |
| | | GO: 7242; intracellular signaling cascade; inferred from | statement” |
| | | electronic annotation” | |
| 3 | Exemplarsequence | “GO: 6260; DNA replication; inferred from electronic annotation | “GO: 5634; nucleus; inferred from electronic annotation |
| | | GO: 6304; DNA modification; inferred from electronic | GO: 5622; BRCT; intracellular; 2.4e−15; |
| | | annotation GO: 6281; DNA repair; inferred from electronic | extended: Unknown” |
| | | annotation GO: 6960; antimicrobial humoral response | |
| | | (sensu Invertebrata); traceable author statement” | |
| 4 | Consensussequence | “GO: 7345; embryogenesis and morphogenesis; traceable author | “GO: 5871; kinesin complex; inferred from electronic |
| | | statement GO: 7048; oncogenesis; traceable | annotation” |
| | | author statement” | |
| 5 | Consensussequence | | |
| 6 | Exemplarsequence | “GO: 7596; blood coagulation; traceable author statement” | |
| 7 | Consensussequence | | |
| 8 | Consensussequence | | |
| 9 | Exemplarsequence | | |
| 10 | Exemplarsequence | | |
| 11 | Consensussequence | | |
| 12 | Consensussequence | “GO: 7345; embryogenesis and morphogenesis; traceable author | “GO: 5871; kinesin complex; inferred from electronic |
| | | statement GO: 7048; oncogenesis; traceable | annotation” |
| | | author statement” | |
| 13 | Exemplarsequence | | |
| 14 | Consensussequence | | |
| 15 | Consensussequence | | |
| 16 | Consensussequence | | |
| 17 | Exemplarsequence | | |
| 18 | Consensussequence | “GO: 6464; protein modification; traceable author statement” | “GO: 5813; centrosome; traceable author statement” |
| 19 | Exemplarsequence | “GO: 6355; regulation of transcription, DNA-dependent; | “GO: 5634; nucleus; inferred from electronic annotation” |
| | | inferred from electronic annotation GO: 6366; transcription from | |
| | | Pol II promoter; traceable author statement” | |
| 20 | Consensussequence | | |
| 21 | Exemplarsequence | “GO: 7283; spermatogenesis; traceable author statement | “GO: 16021; integral to membrane; inferred from |
| | | GO: 6508; proteolysis and peptidolysis; inferred from | electronic annotation” |
| | | electronic annotation” | |
| 22 | Consensussequence | | |
| 23 | Exemplarsequence | | |
| 24 | Consensussequence | | |
| 25 | Consensussequence | | |
| 26 | Consensussequence | “GO: 7275; development; inferred from electronic annotation” | |
| 27 | Exemplarsequence | “GO: 7156; homophilic cell adhesion; inferred from | “GO: 16021; integral to membrane; non-traceable author |
| | | electronic annotation GO: 7416; synaptogenesis; | statement” |
| | | traceable author statement GO: 7273; regulation of synapse; | |
| | | traceable author statement GO: 7155; cell adhesion; | |
| | | non-traceable author statement” | |
| 28 | Consensussequence | | |
| 29 | Consensussequence | “GO: 9405; pathogenesis; traceable author statement” | “GO: 5737; cytoplasm; traceable author statement |
| | | | GO: 5871; kinesin complex; inferred from electronic |
| | | | annotation” |
| 30 | Exemplarsequence | | |
| 31 | Consensussequence | | |
| 32 | Exemplarsequence | “GO: 6357; regulation of transcription from Pol II promoter; | “GO: 5634; nucleus; traceable |
| | | traceable author statement” author statement” | |
| 33 | Consensussequence | “GO: 7222; frizzled receptor signaling pathway; experimental | “GO: 16021; integral to membrane; predicted/computed |
| | | evidence GO: 7048; oncogenesis; predicted/computed” | GO: 5886; plasma membrane; experimental evidence” |
| 34 | Exemplarsequence | “GO: 6259; DNA metabolism; traceable author statement | “GO: 5764; lysosome; traceable author statement” |
| | | GO: 6915; apoptosis; inferred from electronic annotation” | |
| 35 | Exemplarsequence | “GO: 7126; meiosis; traceable author statement GO: 7283; | “GO: 5634; nucleus; traceable author statement” |
| | | spermatogenesis; traceable author statement GO: 7131; meiotic | |
| | | recombination; traceable author statement GO: 7062; sister | |
| | | chromatid cohesion; traceable author statement” | |
| 36 | Exemplarsequence | “GO: 6355; regulation of transcription, DNA-dependent; | “GO: 5634; nucleus; non-traceable author statement” |
| | | non-traceable author statement GO: 7275; development; | |
| | | traceable author statement GO: 8151; cell growth and/or | |
| | | maintenance; inferred from electronic annotation GO: 7048; | |
| | | oncogenesis; traceable author statement” | |
| 37 | Consensussequence | | |
| 38 | Consensussequence | “GO: 6979; response to oxidative stress; traceable author | |
| | | statement” | |
| 39 | Consensussequence | | |
| 40 | Consensussequence | | |
| 41 | Consensussequence | | |
| 42 | Consensussequence | | |
| 43 | Exemplarsequence | “GO: 6950; response to stress; not recorded GO: 6803; | |
| | | glutathione conjugation reaction; inferred from electronic | |
| | | annotation” | |
| 44 | Consensussequence | “GO: 7596; blood coagulation; traceable author statement” | |
| 45 | Consensussequence | | |
| 46 | Consensussequence | | |
| 47 | Consensussequence | | |
| 48 | Consensussequence | | |
| 49 | Consensussequence | | |
| 50 | Consensussequence | | |
| 51 | Consensussequence | | |
| 52 | Exemplarsequence | “GO: 6878; copper ion homeostasis; not recorded GO: 6879; | “GO: 5615; extracellular space; traceable author |
| | | iron ion homeostasis; traceable author statement” | statement” |
| 53 | Exemplarsequence | “GO: 6979; response to oxidative stress; traceable author | |
| | | statement” | |
| 54 | Consensussequence | “GO: 9117; nucleotide metabolism; inferred from electronic | |
| | | annotation” | |
| 55 | Exemplarsequence | “GO: 8643; carbohydrate transport; inferred from electronic | “GO: 5886; plasma membrane; traceable author |
| | | annotation GO: 5975; carbohydrate metabolism; traceable author | statement GO: 16021; integral to membrane; inferred |
| | | statement GO: 15758; glucose transport; traceable author | from electronic annotation” |
| | | statement GO: 15755; fructose transport; traceable author | |
| | | statement” | |
| 56 | Exemplarsequence | | |
| 57 | Consensussequence | | |
| 58 | Exemplarsequence | “GO: 7187; G-protein signaling, coupled to cyclic nucleotide | “GO: 5887; integral to plasma membrane; traceable |
| | | second messenger; traceable author statement” | author statement” |
| 59 | Consensussequence | | |
| 60 | Consensussequence | | |
| 61 | Consensussequence | | |
| 62 | Consensussequence | | |
| 63 | Exemplarsequence | | |
| 64 | Consensussequence | “GO: 6412; protein biosynthesis; inferred from electronic | “GO: 5840; ribosome; inferred from electronic annotation |
| | | annotation” | GO: 5842; cytosolic large ribosomal subunit (sensu |
| | | | Eukarya); not recorded GO: 5622; intracellular; inferred |
| | | | from electronic annotation” |
| 65 | Exemplarsequence | | |
| 66 | Consensussequence | | |
| 67 | Consensussequence | | |
| 68 | Consensussequence | | |
| 69 | Consensussequence | “GO: 7187; G-protein signaling, coupled to cyclic nucleotide | “GO: 5887; integral to plasma membrane; traceable |
| | | second messenger; traceable author statement” | author statement” |
| 70 | Consensussequence | | |
| 71 | Exemplarsequence | | |
| 72 | Consensussequence | | |
| 73 | Consensussequence | “GO: 6355; regulation of transcription, DNA-dependent; inferred | “GO: 5634; nucleus; traceable author statement” |
| | | from electronic annotation GO: 6350; transcription; traceable | |
| | | author statement” | |
| 74 | Consensussequence | “GO: 6979; response to oxidative stress; traceable author | |
| | | statement” | |
| 75 | Exemplarsequence | | |
| 76 | Exemplarsequence | | |
| 77 | Consensussequence | | |
| 78 | Consensussequence | | |
| 79 | Consensussequence | “GO: 7165; signal transduction; non-traceable author statement | “GO: 8180; signalosome complex; inferred from direct |
| | | GO: 6366; transcription from Pol II promoter; traceable author | assay GO: 5737; cytoplasm; inferred from direct assay” |
| | | statement” | |
| 80 | Exemplarsequence | “GO: 6629; lipid metabolism; not recorded GO: 7420; | “GO: 5624; membrane fraction; traceable author |
| | | brain development; traceable author statement GO: 7602; | statement GO: 5622; intracellular; inferred from |
| | | phototransduction; traceable author statement GO: 6810; | electronic annotation” |
| | | transport; inferred from electronic annotation” | |
| 81 | Consensussequence | | |
| 82 | Consensussequence | | |
| 83 | Exemplarsequence | “GO: 7623; circadian rhythm; predicted/computed” | |
| 84 | Exemplarsequence | “GO: 6281; DNA repair; traceable author statement” | “GO: 5634; nucleus; traceable author statement |
| | | | GO: 5643; nuclear pore; traceable author statement” |
| 85 | Consensussequence | | |
| 86 | Consensussequence | | |
| 87 | Consensussequence | | |
| 88 | Exemplarsequence | | |
| 89 | Consensussequence | | |
| 90 | Consensussequence | “GO: 9117; nucleotide metabolism; inferred from electronic | |
| | | annotation” | |
| 91 | Consensussequence | | |
| 92 | Exemplarsequence | “GO: 8202; steroid metabolism; traceable author statement” | |
| 93 | Consensussequence | | |
| 94 | Consensussequence | “GO: 6958; complement activation, classical pathway; inferred | |
| | | from electronic annotation GO: 6508; proteolysis and | |
| | | peptidolysis; inferred from electronic annotation | |
| | | GO: 6955; immune response; traceable author statement” | |
| 95 | Consensussequence | | |
| 96 | Consensussequence | “GO: 1558; regulation of cell growth; inferred from electronic | “GO: 5576; extracellular; inferred from electronic |
| | | annotation” | |
| 97 | Consensussequence | “GO: 6334; nucleosome assembly; inferred from electronic | “GO: 5634; nucleus; inferred from electronic annotation” |
| | | annotation” | |
| 98 | Consensussequence | | |
| 99 | Consensussequence | | |
| 100 | Consensussequence | “GO: 6334; nucleosome assembly; inferred from electronic | “GO: 5634; nucleus; inferred from electronic annotation |
| | | annotation GO: 7001; chromosome organization and biogenesis | GO: 786; nucleosome; inferred from electronic |
| | | (sensu Eukarya); inferred from electronic annotation” | annotation GO: 5694; chromosome; inferred from |
| | | | electronic annotation” |

| | | | Sequence | |
|---|---|---|---|---|
| | Go_Molecular_Function | Transcript ID | Derived From | Sequence ID |
| 1 | | Hs.2384.0 | NM_005079.1 | g4B27037 |
| 2 | “GO: 4383; guanylate cyclase activity; inferred from electronic annotation GO: 8294; | Hs.2352.0 | AU149572 | Hs.2352.0.S1 |
| | calcium/calmodulin- responsive adenylate cyclase activity; inferred from electronic | | | |
| | annotation GO: 16829; lyase activity; inferred from electronic annotation” | | | |
| 3 | “GO: 3890; beta DNA polymerase activity; inferred from electronic annotation | Hs.272537.0 | M11722.1 | g339436 |
| | GO: 3677; DNA binding; not recorded GO: 3912; DNA nucleotidylexotranserase | | | |
| | activity; traceable author statement GO: 16740; transferase activity; inferred from | | | |
| | electronic annotation GO: 287; magnesium ion binding; inferred from electronic | | | |
| | annotation” | | | |
| 4 | | Hs.2384.0 | BE974098 | Hs.2384.0.S2 |
| 5 | | Hs.292815.0 | BG169443 | Hs.292815.0.A1 |
| 6 | “GO: 4867; serine protease inhibitor activity; inferred from electronic annotation GO: 5209; | Hs.170279.1 | AF021834.1 | g4103170 |
| | plasma protein; not recorded GO: 5211; plasma glycoprotein; not recorded” | | | |
| 7 | | Hs.25329.0 | AW043602 | Hs.25329.0.A1 |
| 8 | | Hs.48461.0 | N62126 | Hs.48461.0.A1 |
| 9 | | Hs.42315.1 | AF289495.1 | g11494382 |
| 10 | | g13623228 | BC006212.1 | g13623228 |
| 11 | | Hs.85481.0 | H38035 | Hs.85481.0.A1 |
| 12 | | Hs.2384.0 | BE974098 | Hs.2384.0.S2 |
| 13 | “GO: 3676; KRAB; nucleic acid binding activity: 4.4e−26; extended: inferred from | Hs.192662.0 | NM_018102.1 | g8922439 |
| | electronic annotation” | | | |
| 14 | | Hs.287426.0 | AU144919 | Hs.287426.0 |
| 15 | | Hs.49136.0 | AL039862 | Hs.49136.0.A1 |
| 16 | | Hs.6655.0 | AL355688.1 | Hs.6655.0.S1 |
| 17 | | Hs.330056.0 | NM_025084.1 | g13443001 |
| 18 | | Hs.125682.1 | AI582773 | Hs.125682.1_RC |
| 19 | “GO: 3713; transcription co-activator activity; traceable author statement GO: 3773; | Hs.158195.1 | BC005329.1 | g13529106 |
| | heat shock protein activity; inferred from electronic annotation GO: 3700; transcription | | | |
| | factor activity; traceable author statement” | | | |
| 20 | | Hs.289068.0 | AK021980.1 | Hs.289068.0 |
| 21 | “GO: 4222; metalloendopeptidase activity; inferred from electronic annotation GO: 8270; | Hs.174030.1 | NM_021777.1 | g11496993 |
| | zinc ion binding; inferred from electronic annotation GO: 16787; hydrolase activity; | | | |
| | inferred from electronic annotation” | | | |
| 22 | | Hs.184261.1 | N22898 | Hs.184261.1.A1 |
| 23 | | Hs.306203.0 | NM_017961.1 | g8923685 |
| 24 | | Hs.161353.0 | AI939580 | Hs.161353.0_RC |
| 25 | | Hs.143587.0 | BE550967 | Hs.143587.0.A1 |
| 26 | | Hs.82223.0 | AL049176 | Hs.82223.0 |
| 27 | “GO: 8014; calcium-dependent cell adhesion molecule activity; non-traceable author | Hs.119693.0 | BC001186.1 | g12654692 |
| | statement GO: 5509; calcium ion binding; inferred from electronic annotation” | | | |
| 28 | | Hs.213003.0 | AW203986 | Hs.213003.0.A1 |
| 29 | “GO: 5515; protein binding; traceable author statement” | Hs.24948.1 | AK021944.1 | Hs.24948.1_RC |
| 30 | “GO: 5515; protein binding; inferred from electronic annotation” | Hs.7935.0 | NM_014962.1 | g7662401 |
| 31 | | Hs.102301.0 | AL080215.1 | Hs.102301.0 |
| 32 | “GO: 3677; DNA binding; inferred from electronic annotation GO: 3702; RNA polymerase | Hs.326198.0 | NM_003199.1 | g4507398 |
| | II transcription factor activity; traceable author statement” | | | |
| 33 | “GO: 4928; frizzled receptor activity; experimental evidence GO: 4888; Fz; | Hs.173859.0 | AI333651 | Hs.173859.0 |
| | transmembrane receptor activity; 5.3e−64; extended: Unknown” | | | |
| 34 | “GO: 16787; hydrolase activity; inferred from electronic annotation GO: 3677; | Hs.118243.0 | AB004574.1 | g3184394 |
| | DNA binding; traceable author statement GO: 4519; endonuclease activity; inferred from | | | |
| | electronic annotation GO: 4531; deoxyribonuclease II activity; | | | |
| | traceable author statement” | | | |
| 35 | | Hs.4767.0 | NM_005132.1 | g9845292 |
| 36 | “GO: 3700; transcription factor activity; traceable author statement GO: 5524; ATP binding; | Hs.129914.4 | D43967.1 | g966994 |
| | non-traceable author statement GO: 3677; Runt; DNA binding activity; | | | |
| | 1.2e−102; extended: Unknown” | | | |
| 37 | | Hs.157975.0 | AW298141 | Hs.157975.0.A1 |
| 38 | “GO: 8430; selenium binding; traceable author statement” | Hs.24172.0 | BE856597 | Hs.24172.0.A1 |
| 39 | | Hs.124620.0 | AI907083 | Hs.124620.0_RC |
| 40 | | Hs.78960.0 | AI565177 | Hs.78960.0.A1 |
| 41 | | Hs.306329.0 | AL109684.1 | Hs.306329.0 |
| 42 | | Hs.101550.0 | AW451115 | Hs.101550.0_RC |
| 43 | “GO: 4364; glutathione transferase activity; traceable author statement GO: 16740; | Hs.169907.0 | NM_001512.1 | g4504172 |
| | transferase activity; inferred from electronic annotation” | | | |
| 44 | “GO: 4867; serine protease inhibitor activity; inferred from electronic annotation GO: 5209; | Hs.170279.2 | BF109662 | Hs.170279.2 |
| | plasma protein; not recorded GO: 5211; plasma glycoprotein; not recorded” | | | |
| 45 | | Hs.127217.0 | AI285884 | Hs.127217.0_RC |
| 46 | | Hs.58009.0 | AW449813 | Hs.58009.0.S1 |
| 47 | | Hs.199596.0 | AI671177 | Hs.199596.0.A1 |
| 48 | | Hs.270826.0 | AW008270 | Hs.270826.0_RC |
| 49 | | Hs.132879.0 | R28353 | Hs.132879.0_RC |
| 50 | | Hs.190090.0 | AA629075 | Hs.190090.0_RC |
| 51 | | Hs.194423.0 | AI005638 | Hs.194423.0_RC |
| 52 | “GO: 4323; multicopper ferroxidase iron transport mediator activity; inferred from | Hs.296634.0 | NM_000096.1 | g4557484 |
| | electronic annotation GO: 4322; 16.3.1; ferroxidase activity; 1e−300; extended: | | | |
| | inferred electronic annotation GO: 16491; oxidoreductase activity; inferred from | | | |
| | electronic annotation GO: 5507; copper ion binding; inferred from electronic annotation” | | | |
| 53 | “GO: 8430; selenium binding; traceable author statement” | Hs.3314.0 | NM_005410.1 | g4885590 |
| 54 | “GO: 16787; hydrolase activity; inferred from electronic annotation” | Hs.35198.0 | AA609053 | Hs.35198.0.S2 |
| 55 | “GO: 5351; sugar porter activity; inferred from electronic annotation GO: 5355; | Hs.33084.0 | NM_003039.1 | g4507012 |
| | glucose transporter activity; traceable author statement GO: 5353; fructose transporter | | | |
| | activity; traceable author statement GO: 5215; transporter activity; | | | |
| | inferred from electronic annotation” | | | |
| 56 | | Hs.175982.0 | NM_024826.1 | g13376225 |
| 57 | | Hs.183418.17 | AA601031 | Hs.183418.17 |
| 58 | “GO: 4930; G-protein coupled receptor activity; traceable author statement GO: 4948; | Hs.152175.1 | U17473.1 | g662328 |
| | calcitonin receptor activity; inferred from electronic annotation” | | | |
| 59 | | Hs.5020.0 | AI768384 | Hs.5020.0_RC |
| 60 | | Hs.79732.5 | AI983904 | Hs.79732.5.A1 |
| 61 | | Hs.253594.0 | AI086336 | Hs.253594.0_RC |
| 62 | “GO: 3676; rrm; nucleic acid binding activity; 3.5e−22; extended:inferred from electronic | Hs.103512.0 | BE000929 | Hs.103512.0.A1 |
| | annotation” | | | |
| 63 | | Hs.285130.0 | AF288404.1 | g9800508 |
| 64 | “GO: 3735; structural constituent of ribosome; not recorded GO: 3723; RNA binding; | Hs.296290.0 | AU155515 | Hs.296290.0.A1 |
| | not recorded” | | | |
| 65 | | Hs.128155.0 | NM_014151.1 | g7661757 |
| 66 | | Hs.120850.0 | AA740632 | Hs.120850.0_RC |
| 67 | | 4860484_rc | AI525402 | 4860484_rc |
| 68 | | Hs.24485.1 | AW130559 | Hs.24485.1.A1 |
| 69 | “GO: 4930; G-protein coupled receptor activity; traceable author statement GO: 4948; | Hs.201591.0 | AI478743 | Hs.201591.0_RC |
| | calcitonin receptor activity; inferred from electronic annotation” | | | |
| 70 | | Hs.278004.0 | AW265514 | Hs.278004.0.S1 |
| 71 | | Hs.3821.0 | NM_015678.1 | g7657362 |
| 72 | | Hs.131811.0 | AI017983 | Hs.131811.0.A1 |
| 73 | “GO: 3713; transcription co-activator activity; traceable author statement” | Hs.155017.0 | AI824012 | Hs.155017.0.S1 |
| 74 | “GO: 8430; selenium binding; traceable author statement” | Hs.3314.1 | AV653290 | Hs.3314.1_RC |
| 75 | | Hs.109706.0 | NM_016185.1 | g7705876 |
| 76 | | Hs.143601.0 | AF251294.1 | g12005729 |
| 77 | | Hs.250813.0 | AK022170.1 | Hs.250813.0.S1 |
| 78 | “GO: 3676; rrm; nucleic acid binding activity; 3.5e−22; extended; inferred from | Hs.173179.0 | BF029215 | Hs.173179.0.S1 |
| | electronic annotation” | | | |
| 79 | “GO: 5515; protein binding; traceable author statement GO: 4871; signal transducer | Hs.30212.1 | AA496247 | Hs.30212.1.S2 |
| | activity; non-traceable author statement” | | | |
| 80 | “GO: 8526; phosphatidylinositol transporter activity; traceable author statement GO: 46872; | Hs.93837.0 | NM_004910.1 | g4758925 |
| | metal ion binding; inferred from electronic annotation” | | | |
| 81 | | Hs.34244.0 | AA026388 | Hs.34244.0.A1 |
| 82 | | Hs.239370.0 | AI242661 | Hs.239370.0.S1 |
| 83 | | Hs.153405.0 | NM_022817.1 | g12707561 |
| 84 | “GO: 4840; ubiquitin conjugating enzyme activity; traceable author statement” | Hs.81424.0 | U83117.1 | g1769601 |
| 85 | | Hs.94030.0 | AL110152.1 | Hs.94030.0.S1 |
| 86 | | Hs.134491.0 | AI076185 | Hs.134491.0.A1 |
| 87 | | Hs.23133.1 | BE855713 | Hs.23133.1.S1 |
| 88 | | Hs.82110.0 | AF098482.1 | g4050033 |
| 89 | | Hs.287527.0 | AU148142 | Hs.287527.0.S1 |
| 90 | “GO: 16787; hydrolase activity; inferred from electronic annotation” | Hs.54037.0 | AW194947 | Hs.54037.0 |
| 91 | | Hs.238956.1 | AA046439 | Hs.238956.1.A1 |
| 92 | “GO: 8146; Sulfotransfer; sulfotransferase activity; 7.8e−144; extended:inferred from | Hs.54576.0 | NM_005420.1 | g4885616 |
| | expression pattern GO: 4304; estrone sulfotransferase activity; traceable author statement | | | |
| | GO: 5496; steroid binding; inferred from electronic annotation GO: 16740; | | | |
| | transferase activity; inferred from electronic annotation” | | | |
| 93 | | Hs.143434.2 | AI091445 | Hs.143434.2.A1 |
| 94 | “GO: 3815; complement component C1r activity; traceable author statement GO: 16787; | Hs.1279.1 | AL573058 | Hs.1279.1_RC |
| | hydrolase activity; inferred from electronic annotation GO: 4295; trypsin activity; | | | |
| | inferred from etectronic annotation GO: 4263; chymotrypsin activity; | | | |
| | inferred from electronic annotation GO: 5509; calcium ion binding; inferred | | | |
| | from electronic annotation” | | | |
| 95 | | Hs.323079.1 | BF062299 | Hs.323079.1.A1 |
| 96 | “GO: 8083; growth factor activity; inferred from electronic annotation GO: 5520; | Hs.235935.1 | BF440025 | Hs.235935.1.S1 |
| | insulin-like growth factor binding; inferred from electronic annotation” | | | |
| 97 | “GO: 3677; DNA binding; inferred from electronic annotation” | Hs.284141.0 | AL050331 | Hs.284141.0 |
| 98 | | Hs.43410.0 | N23651 | Hs.43410.0_RC |
| 99 | | Hs.322710.0 | AU146891 | Hs.322710.0.A1 |
| 100 | “GO: 3677; DNA binding; inferred from electronic annotation” | Hs.209228.0 | AW006409 | Hs.209228.0.A1 |

| | Sequence Source | Unigene_Accession | Cluster_Type | LocusLink | Full_Length_Reference_Seq |
|---|---|---|---|---|---|
| 1 | RefSeq | Hs.2384 | fulllength | 7163 | NM_005079; tumor protein D52 |
| 2 | GenBank | Hs.2352 | fulllength | 108 | NM_020546; adenylate cyclase 2 |
| 3 | GenBank | Hs.397294 | fulllength | 1791 | NM_004088; deoxynucleotidyltransferase, terminal |
| 4 | GenBank | Hs.2384 | fulllength | 7163 | NM_005079; tumor protein D52 |
| 5 | GenBank | Hs.372680 | | | |
| 6 | GenBank | Hs.170279 | fulllength | 7035 | NM_006287; tissue factor pathway inhibitor (lipoprotein-associated |
| | | | | | coagulation inhibitor) |
| 7 | GenBank | Hs.172792 | fulllength | 165215 | NM_177454; KIAA1946 protein |
| 8 | GenBank | Hs.32374 | | | |
| 9 | GenBank | Hs.42315 | fulllength | 80321 | NM_024491; p10-binding protein |
| 10 | GenBank | Hs.25298 | fulllength | 84445 | |
| 11 | GenBank | Hs.439144 | est | | |
| 12 | GenBank | Hs.2384 | fulllength | 7163 | NM_005079; tumor protein D52 |
| 13 | RefSeq | Hs.192662 | fulllength | 55713 | NM_018102; zinc finger protein 334 |
| 14 | GenBank | Hs.287426 | | | |
| 15 | GenBank | Hs.49136 | | | |
| 16 | GenBank | Hs.6655 | | | |
| 17 | RefSeq | Hs.288390 | fulllength | 80154 | NM_025084; hypothetical protein FLJ22795 |
| 18 | GenBank | Hs.446494 | fulllength | 56340 | NM_019853; protein phosphatase 4 regulatory subunit 2 |
| 19 | GenBank | Hs.158195 | fulllength | 3298 | NM_004506; heat shock transcription factor 2 |
| 20 | GenBank | Hs.289068 | | | |
| 21 | RefSeq | Hs.174030 | fulllength | 10863 | NM_014265; a disintegrin and metalloproteinase domain 28 isoform 1 |
| | | | | | preproprotein NM_021777; a disintegrin and metalloproteinase domain 28 |
| | | | | | isoform 3 preproprotein NM_021778; a disintegrin and metalloproteinase |
| | | | | | domain 28 isoform 2 preproprotein |
| 22 | GenBank | Hs.184261 | fulllength | 254778 | NM_152765; hypothetical protein MGC33510 |
| 23 | RefSeq | Hs.288995 | fulllength | 55044 | NM_017961; hypothetical protein FLJ20813 |
| 24 | GenBank | Hs.161353 | est | | |
| 25 | GenBank | Hs.143587 | est | | |
| 26 | GenBank | Hs.82223 | fulllength | 91851 | NM_145234; similar to neuralin 1 |
| 27 | GenBank | Hs.119693 | fulllength | 26167 | NM_015669; protocadherin beta 5 precursor |
| 28 | GenBank | Hs.213003 | est | | |
| 29 | GenBank | Hs.24948 | fulllength | 9627 | NM_005460; synuclein alpha interacting protein |
| 30 | RefSeq | Hs.7935 | fulllength | 22903 | NM_014962; BTB/POZ domain containing protein 3 isoform a |
| | | | | | NM_181443; BTB/POZ domain containing protein 3 isoform b |
| 31 | GenBank | Hs.102301 | | | |
| 32 | RefSeq | Hs.326198 | fulllength | 6925 | NM_003199; transcription factor 4 isoform b |
| 33 | GenBank | Hs.173859 | fulllength | 8324 | NM_003507; frizzled 7 |
| 34 | GenBank | Hs.118243 | fulllength | 1777 | NM_001375; deoxyribonuclease II, lysosomal |
| 35 | RefSeq | Hs.4767 | fulllength | 9985 | NM_005132; Rec8p, a meiotic recombination and sister |
| | | | | | chromatid cohesion pho |
| 36 | GenBank | Hs.129914 | fulllength | 861 | NM_001754; runt-related transcription factor 1 (acute myeloid |
| | | | | | leukemia 1; aml1 oncogene) |
| 37 | GenBank | Hs.157975 | | | |
| 38 | GenBank | Hs.275775 | fulllength | 6414 | NM_005410; selenoprotein P precursor |
| 39 | GenBank | Hs.124620 | est | | |
| 40 | GenBank | Hs.436383 | est | | |
| 41 | GenBank | Hs.306329 | | | |
| 42 | GenBank | Hs.17110 | fulllength | 154810 | NM_130847; angiomotin like 1 |
| 43 | RefSeq | Hs.169907 | fulllength | 2941 | NM_001512; glutathione S-transferase A4 |
| 44 | GenBank | Hs.170279 | fulllength | 7035 | NM_006287; tissue factor pathway inhibitor (lipoprotein- |
| | | | | | associated coagulation inhibitor) |
| 45 | GenBank | Hs.127217 | est | | |
| 46 | GenBank | Hs.58009 | | 26050 | |
| 47 | GenBank | Hs.445166 | est | | |
| 48 | GenBank | Hs.370465 | est | | |
| 49 | GenBank | Hs.435763 | est | | |
| 50 | GenBank | Hs.190090 | est | | |
| 51 | GenBank | Hs.194423 | | | |
| 52 | RefSeq | Hs.296634 | fulllength | 1356 | NM_000096; ceruloplasmin (ferroxidase) |
| 53 | RefSeq | Hs.275775 | fulllength | 6414 | NM_005410; selenoprotein P precursor |
| 54 | GenBank | Hs.35198 | fulllength | 59084 | NM_021572; ectonucleotide |
| | | | | | pyrophosphatase/phosphodiesterase 5 (putative function) |
| 55 | RefSeq | Hs.33084 | fulllength | 6518 | NM_003039; solute carrier family 2 (facilitated |
| | | | | | glucose/fructose transporter), member 5 |
| 56 | RefSeq | Hs.61271 | fulllength | 79884 | NM_024826; hypothetical protein FLJ21159 |
| 57 | GenBank | Hs.355702 | est | | |
| 58 | GenBank | Hs.152175 | fulllength | 10203 | NM_005795; calcitonin receptor-like |
| 59 | GenBank | Hs.407068 | | 285378 | |
| 60 | GenBank | Hs.349607 | | 150759 | NM_175853; hypothetical protein LOC150759 |
| 61 | GenBank | Hs.253594 | est | | |
| 62 | GenBank | Hs.103512 | fulllength | 124540 | NM_138962; musashi 2 isoform a NM_170721; musashi 2 |
| | | | | | isoform b |
| 63 | GenBank | Hs.285130 | fulllength | 96626 | NM_033514; pinch-2 |
| 64 | GenBank | Hs.296290 | fulllength | 6168 | NM_000998; ribosomal protein L37a |
| 65 | RefSeq | Hs.128155 | fulllength | 29064 | NM_014151; HSPC053 protein |
| 66 | GenBank | Hs.120850 | est | | |
| 67 | GenBank | Hs.94229 | fulllength | 79732 | NM_024679; hypothetical protein FLJ11939 |
| 68 | GenBank | Hs.146662 | fulllength | 203286 | NM_173551; hypothetical protein FLJ36928 |
| 69 | GenBank | Hs.152175 | fulllength | 10203 | NM_005795; calcitonin receptor-like |
| 70 | GenBank | Hs.278004 | | | |
| 71 | RefSeq | Hs.3821 | fulllength | 26960 | NM_015678; neurobeachin |
| 72 | GenBank | Hs.131811 | est | | |
| 73 | GenBank | Hs.155017 | fulllength | 8204 | NM_003489; receptor interacting protein 140 |
| 74 | GenBank | Hs.275775 | fulllength | 6414 | NM_005410; selenoprotein P precursor |
| 75 | RefSeq | Hs.109706 | fulllength | 51155 | NM_016185; hematological and neurological expressed 1 |
| 76 | GenBank | Hs.143601 | fulllength | 81669 | NM_030937; cyclin L2 |
| 77 | GenBank | Hs.432941 | | | |
| 78 | GenBank | Hs.103512 | fulllength | 124540 | NM_138962; musashi 2 isoform a NM_170721; musashi 2 isoform b |
| 79 | GenBank | Hs.30212 | est | 9318 | NM_004236; thyroid receptor interacting protein 15 |
| 80 | RefSeq | Hs.93837 | fulllength | 9600 | NM_004910; phosphatidylinositol transfer protein, membrane-associated |
| 81 | GenBank | Hs.34244 | est | | |
| 82 | GenBank | Hs.239370 | fulllength | 26086 | NM_015597; activator of G-protein signaling 3 |
| 83 | RefSeq | Hs.153405 | fulllength | 8864 | NM_003894; period 2 isoform 1 NM_022817; period 2 isoform 2 |
| 84 | GenBank | Hs.81424 | fulllength | 7341 | NM_003352; ubiquitin-like 1 (sentrin) |
| 85 | GenBank | Hs.94030 | | | |
| 86 | GenBank | Hs.134491 | est | | |
| 87 | GenBank | Hs.23133 | fulllength | | |
| 88 | GenBank | Hs.351305 | fulllength | 9090 | |
| 89 | GenBank | Hs.287527 | | | |
| 90 | GenBank | Hs.54037 | fulllength | 22875 | NM_014936; ectonucleotide |
| | | | | | pyrophosphatase/phosphodiesterase 4 (putative function) |
| 91 | GenBank | Hs.238956 | | | |
| 92 | RefSeq | Hs.54576 | fulllength | 6783 | NM_005420; sulfotransferase, estrogen-preferring |
| 93 | GenBank | Hs.143434 | fulllength | 1272 | NM_001843; contactin 1 isoform 1 precursor NM_175038; contactin 1 |
| | | | | | isoform 2 precursor |
| 94 | GenBank | Hs.1279 | fulllength | 715 | NM_001733; complement component 1, r subcomponent |
| 95 | GenBank | Hs.323079 | | | |
| 96 | GenBank | Hs.235935 | fulllength | 4856 | NM_002514; nov precursor |
| 97 | GenBank | Hs.284141 | fulllength | 23270 | NM_021648; KIAA0721 protein |
| 98 | GenBank | Hs.43410 | | | |
| 99 | GenBank | Hs.322710 | est | | |
| 100 | GenBank | Hs.143042 | fulllength | 8351 | NM_003530; H3 histone family, member B |

TABLE 17

| | affy id | HUGO | F | p | q | Title | MapLocation | Sequence Type |
|---|---|---|---|---|---|---|---|---|
| 1 | 201691_s_at | TPD52 | 26999 | 2.11e−07 | 6.68e−03 | tumor protein D52 | 8q21 | Exemplarsequence |
| 2 | 242738_s_at | | 32782 | 1.73e−06 | 1.65e−02 | Homo sapiens, clone IMAGE: 5288537, mRNA | | Consensussequence |
| 3 | 213217_at | ADCY2 | 25112 | 2.71e−06 | 1.65e−02 | adenylate cyclase 2 (brain) | 5p15.3 | Consensussequence |
| 4 | 208858_s_at | MBC2 | 24016 | 2.36e−06 | 1.65e−02 | likely ortholog of mouse membrane bound C2 domain | 12q13.13 | Exemplarsequence |
| | | | | | | containing protein | | |
| 5 | 225547_at | | 14885 | 3.02e−06 | 1.65e−02 | Homo sapiens cDNA FLJ39478 fis, clone | | Consensussequence |
| | | | | | | PROST2013605. | | |
| 6 | 226705_at | FGFR1 | 14519 | 3.13e−06 | 1.65e−02 | fibroblast growth factor receptor 1 (fms-related tyrosine | 8p11.2-p11.1 | Consensussequence |
| | | | | | | kinase 2, Pfeiffer syndrome) | | |
| 7 | 201690_s_at | TPD52 | 35309 | 5.63e−06 | 2.55e−02 | tumor protein D52 | 8q21 | Consensussequence |
| 8 | 209610_s_at | SLC1A4 | 23621 | 7.88e−06 | 3.11e−02 | solute carrier family 1 (glutamate/neutral amino acid | 2p15-p13 | Consensussequence |
| | | | | | | transporter), member 4 | | |
| 9 | 210487_at | DNTT | 19968 | 1.11e−05 | 3.20e−02 | deoxynucleotidyltransferase, terminal | 10q23-q24 | Exemplarsequence |
| 10 | 212543_at | AIM1 | 16316 | 9.63e−06 | 3.11e−02 | absent in melanoma 1 | 6q21 | Consensussequence |
| 11 | 201620_at | MBTPS1 | 15950 | 9.81e−06 | 3.11e−02 | membrane-bound transcription factor protease, site 1 | 16q24 | Exemplarsequence |
| 12 | 212811_x_at | PSA | 41883 | 1.42e−05 | 3.75e−02 | phosphoserine aminotransferase | 9q21.2 | Consensussequence |
| 13 | 210665_at | TFPI | 31260 | 2.02e−05 | 4.53e−02 | tissue factor pathway inhibitor (lipoprotein-associated | 2q31-q32.1 | Exemplarsequence |
| | | | | | | coagulation inhibitor) | | |
| 14 | 227370_at | KIAA1946 | 30895 | 2.34e−05 | 4.53e−02 | KIAA1946 protein | 2q32.1 | Consensussequence |
| 15 | 209190_s_at | DIAPH1 | 29434 | 2.10e−05 | 4.53e−02 | diaphanous homolog 1 (Drosophila) | 5q31 | Exemplarsequence |
| 16 | 225832_s_at | LOC221955 | 24320 | 2.55e−05 | 4.53e−02 | KCCR13L | 7p22.2 | Consensussequence |
| 17 | 204499_at | AGTPBP1 | 24320 | 2.51e−05 | 4.53e−02 | ATP/GTP binding protein 1 | 9q21.33 | Consensussequence |
| 18 | 212810_s_at | PSA | 23224 | 2.68e−05 | 4.53e−02 | phosphoserine aminotransferase | 9q21.2 | Consensussequence |
| 19 | 230263_s_at | | 21763 | 2.71e−05 | 4.53e−02 | ESTs | | Consensussequence |
| 20 | 244399_at | | 17380 | 3.19e−05 | 5.05e−02 | ESTs | | Consensussequence |
| 21 | 238520_at | | 14093 | 3.67e−05 | 5.07e−02 | Homo sapiens MSTP020 (MST020) mRNA, complete cds | | Consensussequence |
| 22 | 235721_at | | 12632 | 4.16e−05 | 5.07e−02 | Homo sapiens cDNA FLJ37066 fis, clone | | Consensussequence |
| | | | | | | BRACE2015132, weakly similar to Drosophila | | |
| | | | | | | melanogaster Oregon R cytoplasmic basic protein | | |
| | | | | | | (deltex) mRNA. | | |
| 23 | 232183_at | FLJ14917 | 11902 | 3.98e−05 | 5.07e−02 | hypothetical protein FLJ14917 | 6q25.3 | Consensussequence |
| 24 | 201689_s_at | TPD52 | 46600 | 4.28e−05 | 5.07e−02 | tumor protein D52 | 8q21 | Consensussequence |
| 25 | 208872_s_at | DP1 | 45870 | 4.14e−05 | 5.07e−02 | likely ortholog of mouse deleted in polyposis 1 | 5q22-q23 | Consensussequence |
| 26 | 231874_at | MGC39518 | 45505 | 4.17e−05 | 5.07e−02 | hypothetical protein MGC39518 | 2 | Consensussequence |
| 27 | 225864_at | | 45139 | 4.32e−05 | 5.07e−02 | Homo sapiens cDNA FLJ23705 fis, clone HEP11066. | | Consensussequence |
| 28 | 232081_at | | 43313 | 4.71e−05 | 5.13e−02 | Homo sapiens EST from clone 208499, full insert | | Consensussequence |
| 29 | 215567_at | | 42948 | 4.62e−05 | 5.13e−02 | Homo sapiens cDNA FLJ11574 fis, clone | | Consensussequence |
| | | | | | | HEMBA1003384. | | |
| 30 | 212618_at | KIAA0295 | 42583 | 5.07e−05 | 5.15e−02 | KIAA0295 protein | 15q22.1 | Consensussequence |
| 31 | 220022_at | ZNF334 | 42217 | 4.85e−05 | 5.13e−02 | zinc finger protein 334 | 20q13.12 | Exemplarsequence |
| 32 | 202027_at | C22orf5 | 38146 | 5.28e−05 | 5.15e−02 | chromosome 22 open reading frame 5 | 22q12 | Exemplarsequence |
| 33 | 236922_at | | 38115 | 5.36e−05 | 5.15e−02 | Homo sapiens cDNA FLJ38215 fis, clone | | Consensussequence |
| | | | | | | FCBBF2000291. | | |
| 34 | 226314_at | D4ST-1 | 38054 | 5.57e−05 | 5.19e−02 | dermatan-4-sulfotransferase-1 | | Consensussequence |
| 35 | 214378_at | TFPI | 35612 | 5.89e−05 | 5.33e−02 | tissue factor pathway inhibitor (lipoprotein-associated | 2q31-q32.1 | Consensussequence |
| | | | | | | coagulation inhibitor) | | |
| 36 | 227367_at | | 34151 | 6.31e−05 | 5.56e−02 | ESTs | | Consensussequence |
| 37 | 227391_x_at | | 30498 | 6.97e−05 | 5.87e−02 | ESTs, Weakly similar to hypothetical protein FLJ20294 | | Consensussequence |
| | | | | | | [Homo sapiens] [H. sapiens] | | |
| 38 | 227100_at | | 30133 | 7.04e−05 | 5.87e−02 | Homo sapiens, clone IMAGE: 4825471, mRNA | | Consensussequence |
| 39 | 222491_at | FLJ32731 | 28672 | 7.43e−05 | 6.04e−02 | hypothetical protein FLJ32731 | 8p11.1 | Consensussequence |
| 40 | 243579_at | MSI2 | 28307 | 7.64e−05 | 6.05e−02 | musashi homolog 2 (Drosophila) | 17q23.1 | Consensussequence |
| 41 | 224473_x_at | KIAA1813 | 24654 | 8.47e−05 | 6.55e−02 | KIAA1813 protein | 10q24 | Exemplarsequence |
| 42 | 208873_s_at | DP1 | 24289 | 8.77e−05 | 6.62e−02 | likely ortholog of mouse deleted in polyposis 1 | 5q22-q23 | Exemplarsequence |
| 43 | 201894_s_at | DCN | 22098 | 9.23e−05 | 6.80e−02 | decorin | 12q13.2 | Exemplarsequence |
| 44 | 226164_x_at | | 20637 | 9.72e−05 | 7.00e−02 | Homo sapiens mRNA; cDNA DKFZp586I0521 (from | | Consensussequence |
| | | | | | | clone DKFZp586I0521) | | |
| 45 | 225589_at | POSH | 19906 | 1.08e−04 | 7.04e−02 | likely ortholog of mouse plenty at SH3 domains | 4q32.3 | Consensussequence |
| 46 | 229969_at | | 18810 | 1.05e−04 | 7.04e−02 | ESTs, Moderately similar to KIAA0377 gene product | | Consensussequence |
| | | | | | | [Homo sapiens] [H. sapiens] | | |
| 47 | 244650_at | | 18445 | 1.07e−04 | 7.04e−02 | ESTs | | Consensussequence |
| 48 | 227107_at | | 17349 | 1.11e−04 | 7.04e−02 | ESTs | | Consensussequence |
| 49 | 211709_s_at | SCGF | 17349 | 1.09e−04 | 7.04e−02 | stem cell growth factor; lymphocyte secreted C-type lectin | 19q13.3 | Exemplarsequence |
| 50 | 228813_at | | 17349 | 1.10e−04 | 7.04e−02 | ESTs | | Consensussequence |
| 51 | 235016_at | | 16254 | 1.14e−04 | 7.08e−02 | Homo sapiens, clone IMAGE: 3626729, mRNA | | Consensussequence |
| 52 | 224150_s_at | BITE | 15888 | 1.26e−04 | 7.18e−02 | p10-binding protein | 3q22-q23 | Exemplarsequence |
| 53 | 214582_at | PDE3B | 15523 | 1.19e−04 | 7.15e−02 | phosphodiesterase 3B, cGMP-inhibited | 11p15.1 | Consensussequence |
| 54 | 208033_s_at | ATBF1 | 15523 | 1.18e−04 | 7.15e−02 | AT-binding transcription factor 1 | 16q22.3-q23.1 | Exemplarsequence |
| 55 | 234299_s_at | NIN | 14062 | 1.24e−04 | 7.18e−02 | ninein (GSK3B interacting protein) | 14q21.3 | Consensussequence |
| 56 | 236497_at | | 13697 | 1.34e−04 | 7.20e−02 | ESTs, Weakly similar to cytokine receptor-like factor 2; | | Consensussequence |
| | | | | | | cytokine receptor CRL2 precusor [Homo sapiens] | | |
| | | | | | | [H. sapiens] | | |
| 57 | 244611_at | | 13332 | 1.27e−04 | 7.18e−02 | ESTs, Highly similar to thyroid hormone receptor- | | Consensussequence |
| | | | | | | associated protein, 240 kDa subunit [Homo sapiens] | | |
| | | | | | | [H. sapiens] | | |
| 58 | 226348_at | | 12601 | 1.29e−04 | 7.19e−02 | Homo sapiens cDNA: FLJ23111 fis, clone LNG07835. | | Consensussequence |
| 59 | 223342_at | RRM2B | 12236 | 1.32e−04 | 7.20e−02 | ribonucleotide reductase M2 B (TP53 inducible) | 8q23.1 | Exemplarsequence |
| 60 | 216426_at | | 12236 | 1.38e−04 | 7.22e−02 | | | Consensussequence |
| 61 | 223046_at | EGLN1 | 46935 | 1.39e−04 | 7.22e−02 | egl nine homolog 1 (C. elegans) | 1q42.1 | Consensussequence |
| 62 | 204225_at | HDAC4 | 46935 | 1.41e−04 | 7.22e−02 | histone deacetylase 4 | 2q37.2 | Exemplarsequence |
| 63 | 211220_s_at | HSF2 | 45108 | 1.49e−04 | 7.48e−02 | heat shock transcription factor 2 | 6q22.32 | Exemplarsequence |
| 64 | 223629_at | PCDHB5 | 44743 | 1.67e−04 | 8.03e−02 | protocadherin beta 5 | 5q31 | Exemplarsequence |
| 65 | 225462_at | MGC13159 | 44378 | 1.51e−04 | 7.50e−02 | hypothetical protein MGC13159 | 4p16.2 | Consensussequence |
| 66 | 212385_at | | 42552 | 1.64e−04 | 7.98e−02 | Homo sapiens cDNA FLJ11918 fis, clone | | Consensussequence |
| | | | | | | HEMBB1000272. | | |
| 67 | 201427_s_at | SEPP1 | 38328 | 1.71e−04 | 8.03e−02 | selenoprotein P, plasma, 1 | 5q31 | Exemplarsequence |
| 68 | 235476_at | TSBF1 | 38298 | 1.75e−04 | 8.03e−02 | tumor suppressor TSBF1 | | Consensussequence |
| 69 | 230836_at | | 38298 | 1.91e−04 | 8.03e−02 | Homo sapiens, clone IMAGE: 4816784, mRNA | | Consensussequence |
| 70 | 222217_s_at | SLC27A3 | 38267 | 1.76e−04 | 8.03e−02 | solute carrier family 27 (fatty acid transporter), member 3 | 1q21.1 | Consensussequence |
| 71 | 213119_at | LOC91974 | 38175 | 1.81e−04 | 8.03e−02 | hypothetical protein LOC91974 | 5q33.1 | Consensussequence |
| 72 | 202955_s_at | BIG1 | 38175 | 1.83e−04 | 8.03e−02 | brefeldin A-inhibited guanine nucleotide-exchange protein 1 | 8q13 | Exemplarsequence |
| 73 | 226367_at | | 38114 | 1.94e−04 | 8.03e−02 | Homo sapiens mRNA; cDNA DKFZp434C136 (from clone | | Consensussequence |
| | | | | | | DKFZp434C136) | | |
| 74 | 244539_at | | 38084 | 1.90e−04 | 8.03e−02 | ESTs | | Consensussequence |
| 75 | 226137_at | | 38053 | 1.91e−04 | 8.03e−02 | Homo sapiens, clone IMAGE: 5288537, mRNA | | Consensussequence |
| 76 | 219934_s_at | STE | 38024 | 1.92e−04 | 8.03e−02 | sulfotransferase, estrogen-preferring | 4q13.1 | Exemplarsequence |
| 77 | 235818_at | | 37993 | 1.98e−04 | 8.03e−02 | ESTs, Weakly similar to T09402 immunoglobulin-like | | Consensussequence |
| | | | | | | protein IGSF1 - human [H. sapiens] | | |
| 78 | 214373_at | PPP4R2 | 7.00 | 2.01e−04 | 8.03e−02 | protein phosphatase 4, regulatory subunit 2 | 3q29 | Consensussequence |
| 79 | 225892_at | | 35947 | 2.04e−04 | 8.03e−02 | Homo sapiens mRNA; cDNA DKFZp54D1164 (from | | Consensussequence |
| | | | | | | clone DKFZp564D1164) | | |
| 80 | 203753_at | TCF4 | 35947 | 2.02e−04 | 8.03e−02 | transcription factor 4 | 18q21.1 | Exemplarsequence |
| 81 | 226109_at | C21orf91 | 35582 | 2.08e−04 | 8.03e−02 | chromosome 21 open reading frame 91 | 21q21.1 | Consensussequence |
| 82 | 222600_s_at | FLJ10808 | 35217 | 2.07e−04 | 8.03e−02 | hypothetical protein FLJ10808 | 4q13.2 | Consensussequence |
| 83 | 220602_s_at | FLJ22795 | 34486 | 2.13e−04 | 8.14e−02 | hypothetical protein FLJ22795 | 15q24.3 | Exemplarsequence |
| 84 | 225172_at | CRAMP1L | 32660 | 2.28e−04 | 8.62e−02 | Crm, cramped-like (Drosophila) | 16p13.3 | Consensussequence |
| 85 | 226261_at | LOC223082 | 32660 | 2.32e−04 | 8.66e−02 | LOC223082 | 7p15.1 | Consensussequence |
| 86 | 203948_s_at | MPO | 31199 | 2.38e−04 | 8.72e−02 | myeloperoxidase | 17q23.1 | Exemplarsequence |
| 87 | 224716_at | NFKBIE | 30834 | 2.39e−04 | 8.72e−02 | nuclear factor of kappa light polypeptide gene enhancer | 6p21.1 | Consensussequence |
| | | | | | | in B-cells inhibitor, epsilon | | |
| 88 | 228188_at | FLJ23306 | 30468 | 2.44e−04 | 8.80e−02 | hypothetical protein FLJ23306 | 2p23.3 | Consensussequence |
| 89 | 216266_s_at | BIG1 | 30103 | 2.50e−04 | 8.90e−02 | brefeldin A-inhibited guanine nucleotide-exchange protein 1 | 8q13 | Consensussequence |
| 90 | 243909_x_at | | 29738 | 2.56e−04 | 8.93e−02 | Homo sapiens cDNA FLJ13549 fis, clone | | Consensussequence |
| | | | | | | PLACE1007097. | | |
| 91 | 218599_at | REC8 | 29373 | 2.56e−04 | 8.93e−02 | Rec8p, a meiotic recombination and sister chromatid | 14q11.2-q12 | Exemplarsequence |
| | | | | | | cohesion phosphoprotein of the rad21p family | | |
| 92 | 235516_at | SLA/LP | 28277 | 2.63e−04 | 9.05e−02 | soluble liver antigen/liver pancreas antigen | 4p15.31 | Consensussequence |
| 93 | 235391_at | LOC137392 | 28277 | 2.67e−04 | 9.06e−02 | similar to CG6405 gene product | 8q21.3 | Consensussequence |
| 94 | 227489_at | | 27181 | 2.78e−04 | 9.06e−02 | Homo sapiens cDNA FLJ11157 fis, clone | | Consensussequence |
| | | | | | | PLACE1006961. | | |
| 95 | 219890_at | CLECSF5 | 26451 | 2.87e−04 | 9.06e−02 | C-type (calcium dependent, carbohydrate-recognition | 7q33 | Exemplarsequence |
| | | | | | | domain) lectin, superfamily member 5 | | |
| 96 | 226527_at | | 26085 | 2.83e−04 | 9.06e−02 | ESTs | | Consensussequence |
| 97 | 243010_at | MSI2 | 26085 | 2.88e−04 | 9.06e−02 | musashi homolog 2 (Drosophila) | 17q23.1 | Consensussequence |
| 98 | 237311_at | | 25355 | 2.94e−04 | 9.06e−02 | ESTs | | Consensussequence |
| 99 | 210815_s_at | CALCRL | 24990 | 2.97e−04 | 9.06e−02 | calcitonin receptor-like | 2q32.2 | Exemplarsequence |
| 100 | 211547_s_at | PAFAH1B1 | 24990 | 2.96e−04 | 9.06e−02 | platelet-activating factor acetylhydrolase, isoform lb, | 17p13.3 | Exemplarsequence |
| | | | | | | alpha subunit 45 kDa | | |

| | Go_Biological_Process | Go_Cellular_Component | Go_Molecular_Function |
|---|---|---|---|
| 1 | “GO: 7345; embryogenesis and morphogenesis; traceable | “GO: 5871; kinesin complex; inferred | |
| | author statement GO: 7048; oncogenesis; traceable author | from electronic annotation” | |
| | statement” | | |
| 2 | | | |
| 3 | “GO: 6171; cAMP biosynthesis; non-traceable author | “GO: 16021; integral to membrane; non- | “GO: 4383; guanylate cyclase activity; inferred from electronic |
| | statement GO: 7242; intracellular signaling cascade; inferred | traceable author statement” | annotation GO: 8294; calcium/calmodulin-responsive adenylate cyclase |
| | from electronic annotation” | | activity; inferred from electronic annotation GO: 16829; lyase |
| | | | activity; inferred from electronic annotation” |
| 4 | “GO: 7186; G-protein coupled receptor protein signaling | “GO: 16021; integral to | “GO: 1584; rhodopsin-like receptor activity; inferred from electronic |
| | pathway; inferred from electronic annotation” | membrane; inferred from electronic annotation” | annotation” |
| 5 | | | |
| 6 | “GO: 1501; skeletal development; experimental evidence | “GO: 5887; integral to plasma | “GO: 5007; fibroblast growth factor receptor activity; experimental |
| | GO: 8543; FGF receptor signaling pathway; experimental | membrane; experimental evidence” | evidence” |
| | evidence GO: 165; MAPKKK cascade; experimental evidence | | |
| | GO: 7048; oncogenesis; experimental evidence” | | |
| 7 | “GO: 7345; embryogenesis and morphogenesis; traceable | “GO: 5871; kinesin complex; inferred | |
| | author statement GO: 7048; oncogenesis; traceable author | from electronic annotation” | |
| | statement” | | |
| 8 | “GO: 6835; dicarboxylic acid transport; inferred from electronic | “GO: 5887; integral to plasma | “GO: 5311; sodium; dicarboxylate/tricarboxylate symporter |
| | annotation GO: 15804; neutral amino acid transport; traceable | membrane; traceable author statement | activity; inferred from electronic annotation GO: 15175; neutral amino |
| | author statement GO: 6810; transport; inferred from electronic | GO: 5624; membrane | acid transporter activity; traceable author statement” |
| | annotation” | fraction; traceable author statement” | |
| 9 | “GO: 6260; DNA replication; inferred from electronic | “GO: 5634; nucleus; inferred from | “GO: 3890; beta DNA polymerase activity; inferred from electronic |
| | annotation GO: 6304; DNA modification; inferred from | electronic annotation | annotation GO: 3677; DNA binding; not recorded GO: 3912; DNA |
| | electronic annotation GO: 6281; DNA repair; inferred from | GO: 5622; BRCT; intracellular; 2.4e−15; | nucleotidylexotransferase activity; traceable author statement |
| | electronic annotation GO: 6960; antimicrobial humoral | extended: Unknown” | GO: 16740; transferase activity; inferred from electronic annotation |
| | response (sensu invertebrata); traceable author statement” | | GO: 287; magnesium ion binding; inferred from electronic annotation” |
| 10 | “GO: 7157; heterophilic cell adhesion; inferred from electronic | | “GO: 5529; sugar binding; inferred from electronic annotation” |
| | annotation” | | |
| 11 | “GO: 6629; lipid metabolism; inferred from electronic | “GO: 5788; endoplasmic reticulum | “GO: 8233; peptidase activity; inferred from electronic annotation |
| | annotation GO: 6508; proteolysis and peptidolysis; traceable | lumen; traceable author statement | GO: 4289; subtilase activity; inferred from electronic annotation” |
| | author statement GO: 8203; cholesterol metabolism; inferred | GO: 5794; Golgi apparatus: inferred | |
| | from electronic annotation” | from electronic annotation | |
| | | GO: 16021; integral to | |
| | | membrane; inferred from electronic | |
| | | annotation” | |
| 12 | “GO: 8152; metabolism; inferred from electronic annotation | “GO: 5887; integral to plasma | “GO: 5311; sodium: dicarboxylate/tricarboxylate symporter |
| | GO: 6835; dicarboxylic acid transport; inferred from electronic | membrane; traceable author statement | activity; inferred from electronic annotation GO: 15175; neutral amino |
| | annotation GO: 15804; neutral amino acid transport; traceable | GO: 5624; membrane | acid transporter activity; traceable author statement |
| | author statement GO: 6520; amino acid metabolism; inferred | fraction; traceable author statement” | GO: 4648; phosphoserine transaminase activity; non-traceable author |
| | from electronic annotation GO: 6564; serine biosynthesis; non- | | statement GO: 8483; aminotran_5; transaminase activity; 3.4e−94; |
| | traceable author statement GO: 8615; pyridoxine | | extended: inferred from electronic annotation |
| | biosynthesis; non-traceable author statement | | GO: 4646; 2.6.1.52; phosphoserine aminotransferase activity; 2.71e−127; |
| | GO: 6810; transport; inferred from electronic annotation” | | extended: Unknown GO: 4646; 2.6.1.52; phosphoserine |
| | | | aminotransferase activity; 4.83e−126; extended: Unknown |
| | | | GO: 16740; transferase activity; inferred from electronic annotation |
| | | | GO: 8483; aminotran_5; transaminase activity; 1.3e−127; |
| | | | extended: inferred from electronic annotation” |
| 13 | “GO: 7596; blood coagutation; traceable author statement” | | “GO: 4867; serine protease inhibitor activity; inferred from electronic |
| | | | annotation GO: 5209; plasma protein; not recorded GO: 5211; plasma |
| | | | glycoprotein; not recorded” |
| 14 | | | |
| 15 | “GO: 7605; hearing; traceable author statement” | “GO: 5871; kinesin complex; inferred | “GO: 3779; actin binding; inferred from electronic annotation |
| | | from electronic annotation” | GO: 5102; receptor binding; not recorded GO: 5522; profilin binding; not |
| | | | recorded” |
| 16 | “GO: 6629; lipid metabolism; inferred from electronic | | “GO: 4806; triacylglycerol lipase activity; inferred from electronic |
| | annotation” | | annotation GO: 3824; enzyme activity; inferred from electronic |
| | | | annotation” |
| 17 | | | |
| 18 | “GO: 8152; metabolism; inferred from electronic annotation | “GO: 5887; integral to plasma | “GO: 5311; sodium: dicarboxylate/tricarboxylate symporter |
| | GO: 6835; dicarboxylic acid transport; inferred from electronic | membrane; traceable author statement | activity; inferred from electronic annotation GO: 15175; neutral amino |
| | annotation GO: 15804; neutral amino acid transport; traceable | GO: 5624; membrane | acid transporter activity; traceable author statement |
| | author statement GO: 6520; amino acid metabolism; inferred | fraction; traceable author statement” | GO: 4648; phosphoserine transaminase activity; non-traceable author |
| | from electronic annotation GO: 6564; serine biosynthesis; non- | | statement GO: 8483; aminotran_5; transaminase activity; 3.4e−94; |
| | traceable author statement GO: 8615; pyridoxine | | extended: inferred from electronic annotation |
| | biosynthesis; non-traceable author statement | | GO: 4646; 2.6.1.52; phosphoserine aminotransferase activity; 2.71e−127; |
| | GO: 6810; transport; inferred from electronic annotation” | | extended: Unknown GO: 4646; 2.6.1.52; phosphoserine |
| | | | aminotransferase activity; 4.83e−126; extended: Unknown |
| | | | GO: 16740; transferase activity; inferred from electronic annotation |
| | | | GO: 8483; aminotran_5; transaminase activity; 1.3e−127; |
| | | | extended: inferred from electronic annotation” |
| 19 | | | |
| 20 | | | |
| 21 | | | |
| 22 | | | |
| 23 | | | “GO: 3824; enzyme activity; inferred from electronic annotation” |
| 24 | “GO: 7345; embryogenesis and morphogenesis; traceable | “GO: 5871; kinesin complex; inferred | |
| | author statement GO: 7048; oncogenesis; traceable author | from electronic annotation” | |
| | statement” | | |
| 25 | | “GO: 16021; integral to membrane; non- | |
| | | traceable author statement” | |
| 26 | | | |
| 27 | | | |
| 28 | | | |
| 29 | | | |
| 30 | | | |
| 31 | | | “GO: 3676; KRAB; nucleic acid binding activity; 4.4e−26; |
| | | | extended: inferred from electronic annotation” |
| 32 | | “GO: 16021; integral to | |
| | | membrane; inferred from electronic | |
| | | annotation” | |
| 33 | | | |
| 34 | | | “GO: 16740; transferase activity; inferred from electronic annotation” |
| 35 | “GO: 7596; blood coagulation; traceable author statement” | | “GO: 4867; serine protease inhibitor activity; inferred from electronic |
| | | | annotation GO: 5209; plasma protein; not recorded GO: 5211; plasma |
| | | | glycoprotein; not recorded” |
| 36 | | | |
| 37 | | | |
| 38 | | | |
| 39 | | | |
| 40 | | | “GO: 3676; rrm; nucleic acid binding activity; 3.5e−22; extended: inferred |
| | | | from electronic annotation” |
| 41 | | | |
| 42 | | “GO: 16021; integral to membrane; non- | |
| | | traceable author statement” | |
| 43 | “GO: 7397; histogenesis and organogenesis; traceable author | “GO: 5578; extracellular matrix; not | “GO: 5205; chondroitin sulfate/dermatan sulfate proteoglycan; not |
| | statement” | recorded” | recorded” |
| 44 | | | |
| 45 | | | |
| 46 | | | |
| 47 | | | |
| 48 | | | |
| 49 | “GO: 7267; cell-cell signaling; not recorded GO: 8284; positive | “GO: 5615; extracellular | “GO: 5173; stem cell factor receptor binding; experimental evidence |
| | regulation of cell proliferation; experimental evidence” | space; experimental evidence” | GO: 5529; lectin_c; sugar binding activity; 3.5e−05; extended: Unknown |
| | | | GO: 5530; lectin; predicted/computed” |
| 50 | | | |
| 51 | | | |
| 52 | | | |
| 53 | “GO: 7165; signal transduction; inferred from electronic | “GO: 16020; membrane; inferred from | “GO: 16787; hydrolase activity; inferred from electronic annotation |
| | annotation” | electronic annotation” | GO: 4119; cGMP-inhibited cyclic-nucleotide phosphodiesterase |
| | | | activity; traceable author statement GO: 4114; PDEase; 3′,5′-cyclic- |
| | | | nucleotide phosphodiesterase activity; 9.5e−42; extended: Unknown” |
| 54 | “GO: 6355; regulation of transcription, DNA- | “GO: 5634; nucleus; traceable author | “GO: 3705; RNA polymerase II transcription factor activity, enhancer |
| | dependent; traceable author statement” | statement” | binding; traceable author statement” |
| 55 | | | “GO: 5509; ethand; calcium ion binding |
| | | | activity; 0.036; extended: traceable author statement” |
| 56 | | | |
| 57 | | | |
| 58 | | | |
| 59 | “GO: 9186; deoxyribonucleoside diphosphate | | “GO: 4748; ribonucleoside-diphosphate reductase activity; inferred from |
| | metabolism; inferred from electronic annotation” | | electronic annotation” |
| 60 | | | |
| 61 | “GO: 19538; protein matabolism; inferred from electronic | “GO: 5829; cytosol; traceable author | “GO: 16706; oxidoreductase activity, acting on paired donors, with |
| | annotation” | statement” | incorporation or reduction of molecular oxygen, 2-oxoglutarate as one |
| | | | donor, and incorporation of one atom each of oxygen into both |
| | | | donors; inferred from electronic annotation” |
| 62 | “GO: 7049; cell cycle; non-traceable author statement | “GO: 5634; nucleus; non-traceable | “GO: 4407; histone deacetylase activity; non-traceable author statement |
| | GO: 6355; regulation of transcription, DNA- | author statement” | GO: 16787; hydrolase activity; inferred from electronic annotation |
| | dependent; traceable author statement | | GO: 16564; transcriptional repressor activity; traceable author |
| | GO: 7275; development; non-traceable author statement” | | statement” |
| 63 | “GO: 6355; regulation of transcription, DNA- | “GO: 5634; nucleus; inferred from | “GO: 3713; transcription co-activator activity; traceable author statement |
| | dependent; inferred from electronic annotation | electronic annotation” | GO: 3773; heat shock protein activity; inferred from electronic annotation |
| | GO: 6366; transcription from Pol II promoter; traceable author | | GO: 3700; transcription factor activity; traceable author statement” |
| | statement” | | |
| 64 | “GO: 7156; homophilic cell adhesion; inferred from electronic | “GO: 16021; integral to membrane; non- | “GO: 8014; calcium-dependent cell adhesion molecule activity; non- |
| | annotation GO: 7416; synaptogenesis; traceable author | traceable author statement” | traceable author statement GO: 5509; calcium ion binding; inferred from |
| | statement GO: 7273; regulation of synapse; traceable author | | electronic annotation” |
| | statement GO: 7155; cell adhesion; non-traceable author | | |
| | statement” | | |
| 65 | | | |
| 66 | | | |
| 67 | “GO: 6979; response to oxidative stress; traceable author | | “GO: 8430; selenium binding; traceable aulhor statement” |
| | statement” | | |
| 68 | | “GO: 5622; intracellular; inferred from | “GO: 8270; zinc ion binding; inferred from electronic annotation” |
| | | electronic annotation” | |
| 69 | | | |
| 70 | | | “GO: 3824; AMP-binding; enzyme activity; 8.2e−70; extended: inferred |
| | | | from electronic annotation” |
| 71 | | | |
| 72 | “GO: 6887; exocytosis; traceable author statement” | | “GO: 5085; guanyl-nucleotide exchange factor activity; traceable author |
| | | | statement” |
| 73 | | | |
| 74 | | | |
| 75 | | | |
| 76 | “GO: 8202; steroid metabolism; traceable author statement” | | “GO: 8146; Sulfotransfer; sulfotransferase activity; 7.8e−144; |
| | | | extended: inferred from expression pattern GO: 4304; estrone |
| | | | sulfotransferase activity; traceable author statement GO: 5496; steroid |
| | | | binding; inferred from electronic annotation GO: 16740; transferase |
| | | | activity; inferred from electronic annotation” |
| 77 | | | |
| 78 | “GO: 6464; protein modification; traceable author statement” | “GO: 5813; centrosome; traceable | |
| | | author statement” | |
| 79 | | | |
| 80 | “GO: 6357; regulation of transcription from Pol II | “GO: 5634; nucleus; traceable author | “GO: 3677; DNA binding; inferred from electronic annotation |
| | promoter; traceable author statement” | statement” | GO: 3702; RNA polymerase II transcription factor activity; traceable |
| | | | author statement” |
| 81 | | | |
| 82 | “GO: 6512; ubiquitin cycle; inferred from electronic annotation | | “GO: 3824; enzyme activity; inferred from electronic annotation |
| | GO: 6464; protein modification; inferred from electronic | | GO: 4839; ubiquitin activating enzyme activity; inferred from electronic |
| | annotation” | | annotation” |
| 83 | | | |
| 84 | | | |
| 85 | | | |
| 86 | “GO: 6916; anti-apoptosis; traceable author statement | “GO: 5764; lysosome; traceable author | “GO: 4601; An_peroxidase; peroxidase activity; 6.4e−161; |
| | GO: 6952; defense response; traceable author statement | statement GO: 5634; necleus; traceable | extended: inferred from electronic annotation GO: 3682; chromatin |
| | GO: 6979; response to oxidative stress; traceable author | author statement” | binding; traceable author statement GO: 16687; myeloperoxidase |
| | statement” | | activity; inferred from electronic annotation GO: 16685; eosinophil |
| | | | peroxidase activity; inferred from electronic annotation |
| | | | GO: 5509; calcium ion binding; inferred from electronic annotation |
| | | | GO: 16491; oxidoreductase activity; inferred from electronic annotation |
| | | | GO: 16686; lactoperoxidase activity; inferred from electronic annotation” |
| 87 | | “GO: 5737; cytoplasm; experimental | “GO: 3719; transcription factor binding, cytoplasmic |
| | | evidence” | sequestering; experimental evidence” |
| 88 | | | |
| 89 | “GO: 6887; exocylosis; traceable author statement” | | “GO: 5085; guanyl-nucleotide exchange factor activity; traceable author |
| | | | statement” |
| 90 | | | |
| 91 | “GO: 7126; meiosis; traceable author statement | “GO: 5634; nucleus; traceable author | |
| | GO: 7283; spermatogenesis; traceable author statement | statement” | |
| | GO: 7131; meiotic recombination; traceable author statement | | |
| | GO: 7062; sister chromatid cohesion; traceable author | | |
| | statement” | | |
| 92 | | | “GO: 49; tRNA binding; predicted/computed” |
| 93 | | | |
| 94 | | | |
| 95 | “GO: 7157; heterophilic cell adhesion; inferred from electronic | | “GO: 5529; sugar binding; inferred from electronic annotation” |
| 95 | annotation” | | |
| 96 | | | |
| 97 | | | “GO: 3676; rrm; nucleic acid binding activity; 3.5e−22; extended: inferred |
| | | | from electronic annotation” |
| 98 | | | |
| 99 | “GO: 7187; G-protein signaling, coupled to cyclic nucleotide | “GO: 5887; integral to plasma | “GO: 4930; G-protein coupled receptor activity; traceable author |
| | second messenger; traceable author statement” | membrane; traceable author | statement GO: 4948; calcitonin receptor activity; inferred from electronic |
| | | statement” | annotation” |
| 100 | “GO: 7165; signal transduction; traceable author statement | “GO: 5737; cytoplasm: not recorded” | “GO: 5209; plasma protein; not recorded GO: 16787; hydrolase |
| | GO: 7399; neurogenesis; traceable author statement | | activity; inferred from electronic annotation GO: 3847; 2-acetyl-1- |
| | GO: 6928; cell motility; traceable author statement | | alkylglycerophosphocholine esterase activity; inferred from electronic |
| | GO: 6629; lipid metabolism; traceable author statement” | | annotation” |

| | | Sequence | | Sequence | | | | |
|---|---|---|---|---|---|---|---|---|
| | Transcript ID | Derived From | Sequence ID | Source | Unigene_Accession | Cluster_Type | LocusLink | Full_Length_Reference_Seq |
| 1 | Hs.2384.0 | NM_005079.1 | g4827037 | RefSeq | Hs.2384 | fulllength | 7163 | NM_005079; tumor protein D52 |
| 2 | Hs.163208.0 | BG402859 | Hs.163208.0.A1 | GenBank | Hs.108806 | | | |
| 3 | Hs.2352.0 | AU149572 | Hs.2352.0.S1 | GenBank | Hs.2352 | fulllength | 108 | NM_020546; adenylate cyclase 2 |
| 4 | Hs.8309.0 | BC004998.1 | g13436457 | GenBank | Hs.8309 | fulllength | 23344 | NM_015292; KIAA0747 protein |
| 5 | Hs.292815.0 | BG169443 | Hs.292815.0.A1 | GenBank | Hs.372680 | | | |
| 6 | Hs.748.8 | BE467261 | Hs.748.8.A1 | GenBank | Hs.748 | fulllength | 2260 | NM_000604; fibroblast growth factor receptor 1 isoform 1 precursor |
| | | | | | | | | NM_015850; fibroblast growth factor receptor 1 isoform 2 precursor |
| | | | | | | | | NM_023105; fibroblast growth factor receptor 1 isoform 3 precursor |
| | | | | | | | | NM_023106; fibroblast growth factor receptor 1 isoform 4 precursor |
| | | | | | | | | NM_023107; fibroblast growth factor receptor 1 isoform 5 precursor |
| | | | | | | | | NM_023108; fibroblast growth factor receptor 1 isoform 6 precursor |
| | | | | | | | | NM_023109; fibroblast growth factor receptor 1 isoform 7 precursor |
| | | | | | | | | NM_023110; fibroblast growth factor receptor 1 isoform 8 precursor |
| | | | | | | | | NM_023111; fibroblast growth factor receptor 1 isoform 9 precursor |
| 7 | Hs.2384.0 | BE974098 | Hs.2384.0.S2 | GenBank | Hs.2384 | fulllength | 7163 | NM_005079; tumor protein D52 |
| 8 | Hs.323878.0 | BF340083 | Hs.323878.0 | GenBank | Hs.323878 | fulllength | 6509 | NM_003038; solute carrier family 1, member 4 |
| 9 | Hs.272537.0 | M11722.1 | g339436 | GenBank | Hs.397294 | fulllength | 1791 | NM_004088; deoxynucleotidyltransferase, terminal |
| 10 | Hs.161002.0 | U83115.1 | Hs.161002.0 | GenBank | Hs.161002 | | 202 | |
| 11 | Hs.75890.0 | NM_003791.1 | g4506774 | RefSeq | Hs.75890 | fulllength | 8720 | NM_003791; site-1 protease preproprotein |
| 12 | Hs.286049.2 | BG032165 | Hs.286049.2 | GenBank | Hs.286049 | fulllength | 29968 | NM_003038; solute carrier family 1, member 4 NM_021154; |
| | | | | | | | | phosphoserine aminotransferase isoform 2 NM_058179; |
| | | | | | | | | phosphoserine aminotransferase isoform 1 |
| 13 | Hs.170279.1 | AF021834.1 | g4103170 | GenBank | Hs.170279 | fulllength | 7035 | NM_006287; tissue factor pathway inhibitor (lipoprotein-associated |
| | | | | | | | | coagulation inhibitor) |
| 14 | Hs.25329.0 | AW043602 | Hs.25329.0.A1 | GenBank | Hs.172792 | fulllength | 165215 | NM_177454; KIAA1946 protein |
| 15 | Hs.26584.0 | AF051782.1 | g2947237 | GenBank | Hs.432623 | fulllength | 1729 | NM_005219; diaphanous 1 |
| 16 | Hs.131899.1 | BE795104 | Hs.131899.1_RC | GenBank | Hs.131899 | fulllength | 221955 | NM_139179; KCCR13L |
| 17 | Hs.21542.0 | AB028958.1 | Hs.21542.0 | GenBank | Hs.21542 | fulllength | 23287 | NM_015239; ATP/GTP binding protein 1 |
| 18 | Hs.286049.2 | BG032165 | Hs.286049.2 | GenBank | Hs.286049 | fulllength | 29968 | NM_003038; solute carrier family 1, member 4 NM_021154; |
| | | | | | | | | phosphoserine aminotransferase isoform, 2 NM_058179; |
| | | | | | | | | phosphoserine aminotransferese isoform 1 |
| 19 | Hs.131705.0 | BF447954 | Hs.131705.0.A1 | GenBank | Hs.131705 | est | | |
| 20 | Hs.146119.0 | BG434381 | Hs.146119.0_RC | GenBank | Hs.146119 | est | | |
| 21 | Hs.123364.0 | BF724270 | Hs.123364.0.A1 | GenBank | Hs.123364 | fulllength | | |
| 22 | Hs.48461.0 | N62126 | Hs.48461.0.A1 | GenBank | Hs.32374 | | | |
| 23 | Hs.154706.0 | AA128978 | Hs.154706.0.S1 | GenBank | Hs.154706 | fulllength | 84947 | NM_032861; hypothetical protein FLJ14917 |
| 24 | Hs.2384.0 | BE974098 | Hs.2384.0.S2 | GenBank | Hs.2384 | fulllength | 7163 | NM_005079; tumor protein D52 |
| 25 | Hs.178112.0 | AA814140 | Hs.178112.0.S1 | GenBenk | Hs.178112 | fulllength | 7905 | NM_005669; likely ortholog of mouse deleted in polyposis 1 |
| 26 | Hs.115700.0 | AU155930 | Hs.115700.0.S1 | GenBank | Hs.115700 | fulllength | 285172 | NM_173822; hypothetical protein MGC39518 |
| 27 | Hs.49136.0 | AL039862 | Hs.49136.0.A1 | GenBank | Hs.49136 | | | |
| 28 | Hs.6655.0 | AL355688.1 | Hs.6655.0.S1 | GenBank | Hs.6655 | | | |
| 29 | Hs.287426.0 | AU144919 | Hs.287426.0 | GenBank | Hs.287426 | | | |
| 30 | Hs.155979.0 | AB002293.1 | Hs.155979.0_RC | GenBank | Hs.155979 | | 23060 | |
| 31 | Hs.192662.0 | NM_018102.1 | g8922439 | RefSeq | Hs.192662 | fulllength | 55713 | NM_018102; zinc finger protein 334 |
| 32 | Hs.182626.0 | NM_012264.1 | g7110634 | RefSeq | Hs.182626 | fulllength | 25829 | NM_012264; chromosome 22 open reading frame 5 |
| 33 | Hs.121645.0 | AA772352 | Hs.121645.0_RC | GenBank | Hs.126889 | | | |
| 34 | Hs.24947.0 | AA039350 | Hs.24947.0_RC | GenBank | Hs.24947 | fulllength | 113189 | NM_130468; dermatan-4-sulfotransferase-1 |
| 35 | Hs.170279.2 | BF109662 | Hs.170279.2 | GenBank | Hs.170279 | fulllength | 7035 | NM_006287; tissue factor pathway inhibitor (lipoprotein-associated |
| | | | | | | | | coagulation inhibitor) |
| 36 | Hs.184067.0 | AW976431 | Hs.184067.0 | GenBank | Hs.445376 | est | | |
| 37 | Hs.293327.0 | BE674143 | Hs.293327.0_RC | GenBank | Hs.293327 | est | | |
| 38 | Hs.13205.0 | AI569766 | Hs.13205.0.A1 | GenBank | Hs.13205 | | | |
| 39 | Hs.288057.0 | T56470 | Hs.288057.0 | GenBank | Hs.380474 | fulllength | 138050 | NM_152419; hypothetical protein FLJ32731 |
| 40 | Hs.173179.0 | BF029215 | Hs.173179.0.S1 | GenBank | Hs.103512 | fulllength | 124540 | NM_138962; musashi 2 isoform a NM_170721; musashi 2 isoform b |
| 41 | g13623228 | BC006212.1 | g13623228 | GenBank | Hs.25298 | fulllength | 84445 | |
| 42 | Hs.178112.0 | BC000232.1 | g12652946 | GenBank | Hs.178112 | fulllength | 7905 | NM_005669; likely ortholog of mouse deleted in polyposis 1 |
| 43 | Hs.76152.0 | NM_001920.1 | g4503270 | RefSeq | Hs.433989 | fulllength | 1634 | NM_001920; decorin isoform a preproprotein NM_133503; decorin |
| | | | | | | | | isoform a preproprotein NM_133504; decorin isoform b precursor |
| | | | | | | | | NM_133505; decorin isoform c precursor NM_133506; decorin |
| | | | | | | | | isoform d precursor NM_133507; decorin isoform e precursor |
| 44 | Hs.236463.2 | AI760919 | Hs.236463.2.S1 | GenBank | Hs.367803 | | | |
| 45 | Hs.301804.0 | AB040927.1 | Hs.301804.0 | GenBank | Hs.301804 | | 57630 | |
| 46 | Hs.26904.0 | AV723931 | Hs.26904.0.A1 | GenBank | Hs.26904 | est | | |
| 47 | Hs.152328.0 | AA581439 | Hs.152328.0.A1 | GenBank | Hs.445553 | est | | |
| 48 | Hs.24789.0 | AI807404 | Hs.24789.0.A1 | GenBank | Hs.32163 | est | | |
| 49 | g13543291 | BC005810.1 | g13543291 | GenBank | Hs.105927 | fulllength | 6320 | NM_002975; stem cell growth factor; lymphocyte secreted C-type lectin |
| 50 | Hs.91400.1 | AW206037 | Hs.91400.1.S1 | GenBank | Hs.222874 | est | | |
| 51 | Hs.121557.0 | AL118571 | Hs.121557.0 | GenBank | Hs.121557 | | | |
| 52 | Hs.42315.1 | AF289495.1 | g11494382 | GenBank | Hs.42315 | fulllength | 80321 | NM_024491; p10-binding protein |
| 53 | Hs.150741.3 | NM_000753.1 | Hs.150741.3 | GenBenk | Hs.337616 | fulllength | 5140 | NM_000753; NM_000922; phosphodiesterase 3B, cGMP-inhibited |
| 54 | Hs.101842.0 | NM_006885.1 | g5901893 | RefSeq | Hs.101842 | fulllength | 463 | NM_006885; AT-binding transcription factor 1 |
| 55 | Hs.12772.1 | AK027054.1 | Hs.12772.1 | GenBank | Hs.44054 | fulllength | 51199 | NM_016350; ninein (GSK3B interacting protein) NM_020921; ninein |
| | | | | | | | | (GSK3B interacting protein) |
| 56 | Hs.157489.0 | AI203293 | Hs.157489.0_RC | GenBank | Hs.157489 | est | | |
| 57 | Hs.85481.0 | H38035 | Hs.85481.0.A1 | GenBank | Hs.439144 | est | | |
| 58 | Hs.268231.0 | AK026764.1 | Hs.268231.0 | GenBank | Hs.268231 | | | |
| 59 | Hs.94262.0 | AB036063.1 | g7229085 | GenBank | Hs.94262 | fulllength | 50484 | |
| 60 | Hs.287783.0 | AL136318 | Hs.287783.0.S1 | GenBank | | | | |
| 61 | Hs.6523.1 | NM_022051.1 | Hs.6523.1_RC | GenBank | Hs.6523 | fulllength | 54583 | NM_022051; egl nine homolog 1 |
| 62 | Hs.91400.0 | NM_006037.2 | g13259519 | RefSeq | Hs.91400 | fulllength | 9759 | NM_006037; histone deacetylase 4 |
| 63 | Hs.158195.1 | BC005329.1 | g13529106 | GenBank | Hs.158195 | fulllength | 3298 | NM_004506; heat shock transcription factor 2 |
| 64 | Hs.119693.0 | BC001186.1 | g12654692 | GenBank | Hs.119693 | fulllength | 26167 | NM_015669; protocadherin beta 5 precursor |
| 65 | Hs.12845.0 | AV705805 | Hs.12845.0.S1 | GenBank | Hs.12845 | fulllength | 85013 | NM_032927; hypothetical protein MGC13159 |
| 66 | Hs.289068.0 | AK021980.1 | Hs.289068.0 | GenBank | Hs.289068 | | | |
| 67 | Hs.3314.0 | NM_005410.1 | g4885590 | RefSeq | Hs.275775 | fulllength | 6414 | NM_005410; selenoprotein P precursor |
| 68 | Hs.125759.0 | AW182459 | Hs.125759.0.A1 | GenBank | Hs.125759 | fulllength | 286827 | NM_173084; tumor suppressor TSBF1 |
| 69 | Hs.218289.0 | AI422986 | Hs.218289.0.A1 | GenBank | Hs.152049 | | | |
| 70 | Hs.109274.1 | BC003654.1 | Hs.109274.1 | GenBank | Hs.109274 | fulllength | 11000 | NM_024330; solute carrier family 27 member 3 |
| 71 | Hs.18593.0 | AW058600 | Hs.18593.0_RC | GenBank | Hs.422202 | | 91974 | |
| 72 | Hs.94631.0 | AF084520.1 | g5052120 | GenBank | Hs.94631 | fulllength | 10565 | NM_006421; brefeldin A-inhibited guanine nucleotide-exchange protein 1 |
| 73 | Hs.6567.1 | AL133026.1 | Hs.6567.1 | GenBank | Hs.6567 | | | |
| 74 | Hs.252746.0 | AW665840 | Hs.252746.0_RC | GenBank | Hs.252746 | est | | |
| 75 | Hs.108806.0 | AI288759 | Hs.108806.0.S1 | GenBank | Hs.108806 | | | |
| 76 | Hs.54576.0 | NM_005420.1 | g4885616 | RefSeq | Hs.54576 | fulllength | 6783 | NM_005420 sulfotransferase, estrogen-preferring |
| 77 | Hs.133355.0 | AI498747 | Hs.133355.0.A1 | GenBank | Hs.444431 | est | | |
| 78 | Hs.125682.1 | AI582773 | Hs.125682.1_RC | GenBank | Hs.446494 | fulllength | 56340 | NM_019853; protein phosphatase 4 regulatory subunit 2 |
| 79 | Hs.295789.0 | BF438417 | Hs.295789.0_RC | GenBank | Hs.295789 | | | |
| 80 | Hs.326198.0 | NM_003199.1 | g4507398 | RefSeq | Hs.326198 | fulllength | 6925 | NM_003199; transcription factor 4 isoform b |
| 81 | Hs.49391.1 | AK023825.1 | Hs.49391.1.S2 | GenBank | Hs.49391 | fulllength | 54149 | NM_017447; chromosome 21 open reading frame 91 |
| 82 | Hs.59838.0 | NM_018227.1 | Hs.59838.0 | GenBank | Hs.59838 | fulllength | 55236 | NM_018227; hypothetical protein FLJ10808 |
| 83 | Hs.330056.0 | NM_025084.1 | g13443001 | RefSeq | Hs.288390 | fulllength | 80154 | NM_025084; hypothetical protein FLJ22795 |
| 84 | Hs.15441.0 | AB037847.1 | Hs.15441.0.A1 | GenBank | Hs.15441 | | 57585 | |
| 85 | Hs.127294.0 | AI831561 | Hs.127294.0_RC | GenBank | Hs.127294 | fulllength | 223082 | NM_147128; LOC223082 |
| 86 | Hs.1817.0 | J02694.1 | g189039 | GenBank | Hs.1817 | fulllength | 4353 | NM_000250; myeloperoxidase |
| 87 | Hs.182885.0 | BG163267 | Hs.182855.0.A1 | GenBank | Hs.182885 | fulllength | 4794 | NM_004556; nuclear factor of kappa light polypeptide gene enhancer |
| | | | | | | | | in B-cells inhibitor, epsilon |
| 88 | Hs.5890.0 | AI860150 | Hs.5890.0_RC | GenBank | Hs.5890 | fulllength | 79579 | NM_024530; hypothetical protein FLJ23306 |
| 89 | Hs.94631.1 | AK025637.1 | Hs.94631.1.S1 | GenBank | Hs.94631 | fulllength | 10565 | NM_006421; brefeldin A-inhibited guanine nucleotide-exchange protein 1 |
| 90 | Hs.172745.0 | R43205 | Hs.172745.0_RC | GenBank | Hs.56406 | | | |
| 91 | Hs.4767.0 | NM_005132.1 | g9845292 | RefSeq | Hs.4767 | fulllength | 9985 | NM_005132; Rec8p, a meiotic recombination and sister chromatid cohesion pho |
| 92 | Hs.131209.0 | AI038867 | Hs.131209.0_RC | GenBank | Hs.161436 | fulllength | 51091 | NM_016955; soluble liver antigen/liver pancreas antigen NM_153825; |
| | | | | | | | | soluble liver antigen/liver pancreas antigen |
| 93 | Hs.87672.0 | AW960748 | Hs.87672.0_RC | GenBank | Hs.403869 | fulllength | 137392 | NM_145269; similar to CG6405 gene product |
| 94 | Hs.169872.0 | BE962027 | Hs.169872.0.A1 | GenBank | Hs.323849 | | | |
| 95 | Hs.126355.0 | NM_013252.1 | g10281668 | RefSeq | Hs.126355 | fulllength | 23601 | NM_013252; C-type (calcium dependent, carbohydrate-recognition domain) |
| | | | | | | | | lectin, superfamily member 5 |
| 96 | Hs.5669.0 | AI569785 | Hs.5669.0_RC | GenBank | Hs.5669 | est | | |
| 97 | Hs.103512.0 | BE000929 | Hs.103512.0.A1 | GenBank | Hs.103512 | fulllength | 124540 | NM_138962; musashi 2 isoform a NM_170721; musashi 2 isoform b |
| 98 | Hs.161353.0 | AI939580 | Hs.161353.0_RC | GenBank | Hs.161353 | est | | |
| 99 | Hs.152175.1 | U17473.1 | g662328 | GenBank | Hs.152175 | fulllength | 10203 | NM_005795; calcitonin receptor-like |
| 100 | Hs.77318.1 | L13387.1 | g349827 | GenBank | Hs.77318 | fulllength | 5048 | NM_000430; platelet-activating factor acetylhydrolase, isoform Ib. |
| | | | | | | | | alpha subunit (45 kD) |

TABLE 18
t(8;16) versus WHO
1(8;16) vere(8:16) AML was compared to the 4 other balanced subtypes according to the WHO classification (t(15;17), t(8;21), inv(16), and 11q23/MLL-rearrangements
Top 100 differentially expressed genes:

| Number | Affymetrix_ID | HUGO name | fc | p | q | sfn | t | Title | MapLocation | Sequence Type |
|---|---|---|---|---|---|---|---|---|---|---|
| 1 | 223299_at | LOC90701 | 2.21 | 1.27E−39 | 1.12E−35 | 1.96 | 25.73 | similar to signal peptidase complex (18 kD) | 18q21.31 | Exemplarsequence |
| 2 | 204236_at | FLI1 | 2.34 | 2.39E−32 | 9.01E−29 | 1.95 | 25.19 | Friend leukemia virus integration 1 | 11q24.1-q24.3 | Exemplarsequence |
| 3 | 203409_at | DDB2 | 3.04 | 4.00E−07 | 1.58E−05 | 2.85 | 25.18 | damage-specific DNA binding protein 2, 48 kDa | 11p12-p11 | Exemplarsequence |
| 4 | 223098_s_at | MGC4840 | 2.22 | 1.08E−09 | 7.60E−08 | 2.09 | 22.04 | hypothetical protein MGC4840 | 16q11.2 | Consensussequence |
| 5 | 214937_x_at | PCM1 | 2.48 | 1.80E−05 | 0.000439408 | 2.74 | 20.23 | pericentriolar material 1 | 8p22-p21.3 | Consensussequence |
| 6 | 212786_at | KIAA0350 | 1.87 | 4.38E−08 | 2.12E−06 | 1.96 | 19.57 | KIAA0350 protein | 16p13.2 | Consensussequence |
| 7 | 202174_s_at | PCM1 | 2.40 | 1.90E−05 | 0.000457515 | 2.56 | 19.26 | pericentriolar material 1 | 8p22-p21.3 | Exemplarsequence |
| 8 | 229045_at | MGC35578 | 2.75 | 6.63E−08 | 3.09E−06 | 1.93 | 19.16 | hypothetical protein MGC35578 | 16q11.2 | Consensussequence |
| 9 | 219951_s_at | C20orf12 | 3.56 | 1.67E−05 | 0.000409408 | 2.41 | 18.63 | chromosome 20 open reading frame 12 | 20p11.23 | Exemplarsequence |
| 10 | 231982_at | | −85.92 | 1.95E−43 | 5.14E−39 | −1.37 | −18.45 | Homo sapiens HSPC323 mRNA, partial cds | | Consensussequence |
| 11 | 223000_s_at | F11R | −5.11 | 7.97E−12 | 8.10E−10 | −1.58 | −18.13 | F11 receptor | 1q21.2-q21.3 | Exemplarsequence |
| 12 | 201135_at | ECHS1 | 2.43 | 5.14E−05 | 0.001083676 | 2.63 | 18.07 | enoyl Coenzyme A hydralase, short chain, 1, mitochondrial | 10q26.2-q26.3 | Exemplarsequence |
| 13 | 204924_at | TLR2 | 2.72 | 1.18E−05 | 0.00030409 | 2.24 | 18.00 | toll-like receptor 2 | 4q32 | Exemplarsequence |
| 14 | 200749_at | RAN | 2.55 | 1.69E−05 | 0.000413171 | 2.27 | 17.84 | RAN, member RAS oncogene family | 6p21 | Consensussequence |
| 15 | 205588_s_at | FOP | 2.09 | 4.73E−06 | 0.000137838 | 2.07 | 17.82 | FGFR1 oncogene partner | 6q27 | Exemplarsequence |
| 16 | 235109_at | | −20.99 | 2.18E−40 | 2.87E−36 | −1.27 | −17.30 | Homo sapiens cDNA FLJ40581 fis, clone THYMU2007729. | | Consensussequence |
| 17 | 203584_at | KIAA0103 | 2.75 | 4.57E−06 | 0.000133732 | 1.98 | 17.23 | KIAA0103 gene product | 8q23.1 | Exemplarsequence |
| 18 | 217834_s_at | NSAP1 | 3.91 | 0.000182471 | 0.003119077 | 3.16 | 17.19 | NS1-associated protein 1 | 6q14-q15 | Exemplarsequence |
| 19 | 211626_x_at | ERG | −6.28 | 1.76E−11 | 1.70E−09 | −1.50 | −17.16 | v-ets erythroblastosis virus E26 oncogene like (avian) | 21q22.3 | Exemplarsequence |
| 20 | 223358_s_at | | 3.06 | 1.53E−05 | 0.000378624 | 2.13 | 17.15 | Homo sapiens cDNA FLJ33024 fis, clone | | Consensussequence |
| | | | | | | | | THYMU1000532, moderately similar to HIGH- | | |
| | | | | | | | | AFFINITY CAMP-SPECIFIC 3′,5′-CYCLIC | | |
| | | | | | | | | PHOSPHODIESTERASE (EC 3.1.4.17). | | |
| 21 | 202016_at | MEST | −8.87 | 2.45E−24 | 2.33E−21 | −1.32 | −17.07 | mesodemn specific transcript homolog (mouse) | 7q32 | Exemplarsequence |
| 22 | 209349_at | RAD50 | 1.85 | 3.61E−08 | 1.78E−09 | 1.63 | 16.89 | RAD50 homolog (S. cerevisiae) | 5q31 | Exemplarsequence |
| 23 | 202746_at | ITM2A | −28.95 | 7.47E−38 | 4.92E−34 | −1.20 | −16.41 | integral membrane protein 2A | Xq13.3-Xq21.2 | Consensussequence |
| 24 | 226384_at | HTPAP | −4.04 | 4.58E−37 | 2.42E−33 | −1.19 | −16.23 | HTPAP protein | 8p11.21 | Consensussequence |
| 25 | 232693_s_at | LOC55893 | −11.91 | 1.21E−15 | 2.57E−13 | −1.32 | −16.17 | papillomavirus regulatory factor PRF-1 | 8p21.1 | Consensussequence |
| 26 | 226846_at | LOC254295 | −6.62 | 4.11E−21 | 2.35E−18 | −1.25 | −16.03 | hypothetical protein LOC254295 | 9q34.13 | Consensussequence |
| 27 | 229693_at | | −4.06 | 4.36E−15 | 8.37E−13 | −1.31 | −15.98 | ESTs | | Consensussequence |
| 28 | 223132_s_at | TRIMS | 3.46 | 0.000257941 | 0.004158193 | 3.03 | 15.94 | tripartite motif-containing 8 | 10q24.3 | Exemplarsequence |
| 29 | 237255_at | | 3.45 | 1.71E−05 | 0.000418698 | 1.94 | 15.90 | ESTs. Weakly similar to hypothetical protein | | Consensussequence |
| | | | | | | | | FLJ20489 [Homo sapiens] [H. sapiens] | | |
| 30 | 227019_at | | 1.94 | 1.26E−09 | 8.77E−08 | 1.42 | 15.69 | Homo sapiens cDNA FLJ13137 fis, clone NT2RP3003150. | | Consensussequence |
| 31 | 218455_at | NFS1 | 1.61 | 6.66E−08 | 3.10E−06 | 1.51 | 15.59 | NFS1 nitrogen fixation 1 (S. cerevisiase) | 20q11.21 | Exemplarsequence |
| 32 | 224378_x_at | MAP1LC3A | 2.03 | 1.39E−06 | 4.77E−05 | 1.64 | 15.56 | microtubule-associated protein 1 light chain 3 | 20cen-q13 alpha | Exemplarsequence |
| 33 | 202747_s_at | ITM2A | −43.58 | 4.71E−35 | 2.07E−31 | −1.13 | −15.45 | integral membrane protein 2A | Xq13.3-Xq21.2 | Exemplarsequence |
| 34 | 202895_s_at | PTPNS1 | 1.61 | 8.44E−08 | 3.86E−08 | 1.49 | 15.36 | protein tyrosine phosphatase, non-receptor type substrate 1 | 20p13 | Exemplarsequence |
| 35 | 204423_at | MKLN1 | 2.32 | 5.03E−05 | 0.001061742 | 2.03 | 15.30 | muskelin 1, intracellular mediator containing | 7q32 | Exemplarsequence |
| | | | | | | | | kelch motifs | | |
| 36 | 223284_at | KLP1 | −2.43 | 9.05E−27 | 1.40E−23 | −1.15 | −15.17 | K562 cell-derived leucine-zipper-like protein 1 | 19q13.42 | Exemplarsequence |
| 37 | 213206_at | GOSR2 | 1.91 | 3.92E−10 | 2.98E−08 | 1.33 | 15.09 | golgi SNAP receptor complex member 2 | 17q21 | Consensussequence |
| 38 | 228943_at | | −2.20 | 2.60E−29 | 6.86E−26 | −1.12 | −14.97 | ESTs. Weakly similar to JC5963 stable tubule | | Consensussequence |
| | | | | | | | | only polypeptide - mouse [M. musculus] | | |
| 39 | 208628_s_at | NSEP1 | 1.22 | 5.06E−10 | 3.80E−08 | 1.32 | 14.89 | nuclease sensitive element binding protein 1 | 1p34 | Exemplarsequence |
| 40 | 208889_s_at | NCOR2 | −4.70 | 3.34E−19 | 1.35E−16 | −1.16 | −14.83 | nuclear receptor co-repressor 2 | 12q24 | Consensussequence |
| 41 | 204117_at | PREP | 2.19 | 7.21E−05 | 0.00144616 | 2.02 | 14.83 | prolyl endopeptidase | 6q22 | Exemplarsequence |
| 42 | 200723_s_at | M11S1 | 2.10 | 0.000205473 | 0.003445202 | 2.40 | 14.74 | membrane component, chromosome 11, | 11p13 | Exemplarsequence |
| | | | | | | | | surface marker 1 | | |
| 43 | 216247_at | RPS20 | −2.06 | 9.16E−32 | 3.01E−28 | −1.09 | −14.74 | ribosomal protein S20 | 8q12 | Consensussequence |
| 44 | 203469_s_at | CDK10 | 3.05 | 3.41E−05 | 0.000760347 | 1.80 | 14.47 | cyclin-dependent kinase (CDC2-like) 10 | 16q24 | Exemplarsequence |
| 45 | 202343_x_at | COX5B | 1.65 | 1.03E−06 | 3.60E−05 | 1.48 | 14.42 | cytochrome c oxidase subunit Vb | 2cen-q13 | Exemplarsequence |
| 46 | 212513_s_at | VDU1 | 2.19 | 2.47E−05 | 0.00057428 | 1.73 | 14.35 | pVHL-interacting deubiquitinating enzyme 1 | 1p31.1 | Consensussequence |
| 47 | 220218_at | FLJ10058 | −6.00 | 1.47E−24 | 1.62E−21 | −1.08 | −14.33 | hypothetical protein FLJ10058 | 9p24.1 | Exemplarsequence |
| 48 | 238660_at | | 1.71 | 2.32E−11 | 2.16E−09 | 1.22 | 14.32 | ESTs | | Consensussequence |
| 49 | 209036_s_at | MDH2 | 1.58 | 3.22E−06 | 9.88E−05 | 1.52 | 14.27 | malate dehydrogenase 2, NAD (mitochondrial) | 7p12.3-q11.2 | Exemplarsequence |
| 50 | 223916_s_at | BCoR | 3.10 | 0.000356085 | 0.005381367 | 2.61 | 14.17 | BCL-6 interacting corepressor | Xp11.4 | Exemplarsequence |
| 51 | 211800_s_at | USP4 | 1.98 | 0.000293496 | 0.00460455 | 2.44 | 14.13 | ubiquitin specific protease 4 (proto-oncogene) | 3p21.3 | Exemplarsequence |
| 52 | 201480_s_at | SUPT5H | 1.31 | 4.01E−08 | 1.95E−06 | 1.31 | 14.01 | suppressor of Ty 5 homolog (S. cerevisiae) | 19q13 | Exemplarsequence |
| 53 | 219143_s_at | FLJ20374 | 2.83 | 9.96E−05 | 0.001900448 | 1.93 | 13.99 | hypothetical protein FLJ20374 | 15q22.33 | Exemplarsequence |
| 54 | 209103_s_at | UFD1L | 1.46 | 5.13E−13 | 6.59E−11 | 1.15 | 13.95 | ubiquitin fusion degradation 1-like | 22q11.21 | Exemplarsequence |
| 55 | 222508_s_at | FLJ10154 | 2.38 | 5.93E−06 | 0.000167272 | 1.51 | 13.89 | hypothetical protein FLJ10154 | 13q33.1 | Consensussequence |
| 56 | 227629_at | | 4.05 | 0.000324762 | 0.004973627 | 2.42 | 13.88 | Homo sapiens cDNA FLJ13603 fis, clone | | Consensussequence |
| | | | | | | | | PLACE1010270. | | |
| 57 | 216569_at | | −4.45 | 4.09E−28 | 8.29E−25 | −1.03 | −13.84 | | | Consensussequence |
| 58 | 235154_at | TAF3 | 1.55 | 2.41E−10 | 1.92E−08 | 1.20 | 13.83 | TAF3 RHA polymerase II, TATA box binding | 10p15.1 | Consensussequence |
| | | | | | | | | protein (TBP)-associated factor, 140 kDa | | |
| 59 | 209003_at | SLC25A11 | 1.55 | 3.53E−09 | 2.26E−07 | 1.23 | 13.76 | solute cerrier family 25 (mitochondrial carrier; | 17p13.3 | Exemplarsequence |
| | | | | | | | | oxoglutarate carrier), member 11 | | |
| 60 | 214522_x_at | HIST1H2AD | 4.03 | 4.71E−05 | 0.001004799 | 1.72 | 13.74 | histone 1, H2ad | 6p21.3 | Consensussequence |
| 61 | 221942_s_at | GUCY1A3 | −6.55 | 4.16E−29 | 9.95E−26 | −1.01 | −13.70 | guanylate cyclase 1, soluble. alpha 3 | 4q31.1-q31.2 | Consensussequence |
| 62 | 225264_at | LOC57038 | 1.96 | 0.000133494 | 0.002415101 | 1.94 | 13.67 | similar to arginyl-tRNA synthetase | 6q16.1 | Consensussequence |
| 63 | 243023_at | | −5.77 | 3.07E−19 | 1.26E−16 | −1.06 | −13.66 | ESTs | | Consensussequence |
| 64 | 239641_at | | 7.93 | 0.000821675 | 0.010516972 | 4.94 | 13.65 | Homo sapiens, clone IMAGE: 5173389, mRNA | | Consensussequence |
| 65 | 227923_at | SHANK3 | −48.32 | 1.19E−29 | 3.49E−26 | −1.01 | −13.65 | SH3 and multiple ankyrin repeat domains 3 | 22q13.3 | Consensussequence |
| 66 | 212501_at | CEBPB | 2.08 | 4.39E−05 | 0.000945314 | 1.69 | 13.61 | CCAAT/enhancer binding protein (C/EBP), beta | 20q13.1 | Consensussequence |
| 67 | 210465_s_at | SNAPC3 | 2.47 | 0.000152074 | 0.002693897 | 1.95 | 13.53 | small nuclear RNA activating complex, | 9p22.2 | Exemplarsequence |
| | | | | | | | | polypeptide 3, 50 kDa | | |
| 68 | 201051_at | ANP32A | 2.21 | 0.000299477 | 0.004670572 | 2.23 | 13.48 | acidic (leucine-rich) nuclear phosphoprotein 32 | 15q22.3-q23 | Consensussequence |
| | | | | | | | | family, member A | | |
| 69 | 223204_at | DKFZp434L142 | 13.19 | 0.000741924 | 0.009717629 | 3.60 | 13.47 | hypothetical protein DKFZp434L142 | 4q32.1 | Exemplarsequence |
| 70 | 219014_at | PLAC8 | 2.33 | 0.000123533 | 0.002269182 | 1.86 | 13.43 | placenta-specific 8 | 4q21.3 | Exemplarsequence |
| 71 | 202896_s_at | PTPNS1 | 3.14 | 0.000525082 | 0.007368337 | 2.70 | 13.40 | protein tyrosine phosphatase, non-receptor type | 20p13 | Exemplarsequence |
| | | | | | | | | substrate 1 | | |
| 72 | 208624_s_at | EIF4G1 | 1.99 | 8.54E−05 | 0.001666663 | 1.76 | 13.34 | eukaryotic translation initiation factor 4 gamma, 1 | 3q27-qter | Consensussequence |
| 73 | 223162_s_at | LCHN | −3.40 | 7.03E−07 | 2.63E−05 | −1.32 | −13.30 | LCHN protein | 7q34 | Exemplarsequence |
| 74 | 201015_s_at | JUP | −13.84 | 1.13E−17 | 3.46E−15 | −1.04 | −13.29 | junction plakoglobin | 17q21 | Exemplarsequence |
| 75 | 228391_at | | 2.39 | 9.64E−05 | 0.001847275 | 1.77 | 13.29 | Homo sapiens, Similar to expressed sequence | | Consensussequence |
| | | | | | | | | AW111961, clone IMAGE: 5268751, mRNA | | |
| 76 | 201687_s_at | API5 | 1.58 | 9.82E−18 | 3.10E−15 | 1.04 | 13.27 | apoptosis inhibitor 5 | 11p12-q12 | Exemplarsequence |
| 77 | 226452_at | PDK1 | −3.15 | 5.66E−13 | 7.16E−11 | −1.09 | −13.26 | pyruvate dehydrogenase kinase, isoenzyme 1 | 2q31.1 | Consensussequence |
| 78 | 227600_at | | 2.16 | 1.71E−05 | 0.000416898 | 1.51 | 13.24 | ESTs, Weakly similar to hypothetical protein | | Consensussequence |
| | | | | | | | | FLJ20378 [Homo sapiens] [H. sapiens] | | |
| 79 | 220690_s_at | DKFZp566O084 | 3.44 | 0.000376385 | 0.005616049 | 2.30 | 13.24 | DKFZP566O084 protein | 17p12 | Exemplarsequence |
| 80 | 209494_s_at | ZNF278 | 1.57 | 1.79E−05 | 0.000436202 | 1.51 | 13.21 | zinc finger protein 278 | 22q12.2 | Consensussequence |
| 81 | 208469_s_at | C6orf8 | −4.40 | 1.02E−15 | 2.22E−13 | −1.05 | −13.20 | chromosome 6 open reading frame 8 | 6p21.31 | Exemplarsequence |
| 82 | 241133_at | TRB | −32.67 | 2.41E−28 | 5.28E−25 | −0.97 | −13.18 | T cell receptor beta locus | 7q34 | Consensussequence |
| 83 | 222425_s_at | DKFZP586F1524 | 2.11 | 3.37E−05 | 0.000754482 | 1.57 | 13.17 | DKFZP586F1524 protein | 17q11.1 | Consensussequence |
| 84 | 224863_at | | 2.53 | 7.80E−05 | 0.001551746 | 1.70 | 13.17 | Homo sapiens cDNA FLJ31057 fis, clone | | Consensussequence |
| | | | | | | | | HSYRA2000787. | | |
| 85 | 217829_s_at | SAD1 | 1.73 | 1.69E−05 | 0.000413171 | 1.49 | 13.14 | SnRNP assembly defective 1 homolog | 2p11.2 | Exemplarsequence |
| 86 | 243500_at | | −4.83 | 2.31E−27 | 4.06E−24 | −0.96 | −13.08 | ESTs | | Consensussequence |
| 87 | 212922_s_at | HSKM-B | −2.88 | 3.41E−06 | 0.000103948 | −1.36 | −13.02 | HSKM-B protein | 1q32.3 | Consensussequence |
| 88 | 209710_at | MGC2306 | −7.84 | 8.03E−28 | 1.51E−24 | −0.95 | −13.01 | hypothetical protein MGC2306 | 3q21.3 | Consensussequence |
| 89 | 217803_at | GOLPH3 | 2.42 | 0.000445393 | 0.006439179 | 2.34 | 12.99 | golgi phosphoprotein 3 (coat-protein) | 5p13.3 | Exemplarsequence |
| 90 | 205051_s_at | KIT | −10.71 | 1.56E−17 | 4.66E−15 | −1.01 | −12.95 | v-kit Hardy-Zuckerman 4 feline sarcoma viral | 4q11-q12 | Exemplarsequence |
| | | | | | | | | oncogene homolog | | |
| 91 | 244081_at | | −3.25 | 3.64E−16 | 8.79E−14 | −1.02 | −12.93 | ESTs | | Consensussequence |
| 92 | 214396_s_at | MBD2 | −3.16 | 2.38E−18 | 8.03E−16 | −1.00 | −12.93 | methyl-CpG binding domain protein 2 | 18q21 | Consensussequence |
| 93 | 235142_at | MGC17919 | −7.98 | 2.82E−27 | 4.64E−24 | −0.95 | −12.93 | hypothetical protein MGC17919 | 1p34.3 | Consensussequence |
| 94 | 233749_at | | −7.23 | 3.14E−20 | 1.51E−17 | −0.98 | −12.86 | EST | | Consensussequence |
| 95 | 212380_at | KIAA0082 | 1.44 | 8.52E−09 | 4.89E−07 | 1.15 | 12.80 | KIAA0082 protein | 6p21.2 | Consensussequence |
| 96 | 215736_at | | −6.48 | 4.97E−26 | 6.88E−23 | −0.95 | −12.79 | Homo sapiens clone 23892 mRNA sequence | | Consensussequence |
| 97 | 218377_s_at | C21orf6 | 2.61 | 0.000497369 | 0.007036144 | 2.34 | 12.76 | chromosome 21 open reading frame 6 | 21q22.11 | Exemplarsequence |
| 98 | 225932_s_at | | 1.41 | 1.09E−07 | 4.86E−06 | 1.19 | 12.73 | Homo sapiens, clone IMAGE: 4812021, mRNA | | Consensussequence |
| 99 | 222323_at | | −2.93 | 5.49E−25 | 7.23E−22 | −0.94 | −12.71 | ESTs, Moderately similar to crystallin, gamma | | Consensussequence |
| | | | | | | | | D; gamma crystallin 4 [Homo sapiens] | | |
| | | | | | | | | [H. sapiens] | | |
| 100 | 213541_s_at | ERG | −4.73 | 1.16E−23 | 9.83E−21 | −0.94 | −12.56 | v-ets erythroblastosis virus E26 oncogene like | 21q22.3 | Consensussequence |
| | | | | | | | | (avian) | | |

| | | Sequence | | Sequence | | | | |
|---|---|---|---|---|---|---|---|---|
| Number | Transcript ID | Derived From | Sequence ID | Source | Unigene_Accession | Cluster_Type | LocusLink | Full_Length_Reference_Seq |
| 1 | Hs.68644.0 | AF212233.1 | g13182746 | GenBank | Hs.68644 | fulllength | 90701 | NM_033280; similar to signal peptidase complex (18 kD) |
| 2 | Hs.108043.0 | NM_002017.2 | g7110592 | RefSeq | Hs.108043 | fulllength | 2313 | NM_002017; Friend leukemia virus integration 1 |
| 3 | Hs.77602.0 | NM_000107.1 | g4557514 | RefSeq | Hs.77602 | fulllength | 1643 | NM_000107; damage-specific DNA binding protein 2 (48 kD) |
| 4 | Hs.301872.0 | AA195024 | Hs.301872.0 | GenBank | Hs.301872 | fulllength | 83752 | NM_031490; hypothetical protein MGC4840 |
| 5 | Hs.75737.2 | AI924817 | Hs.75737.2.S1 | GenBank | Hs.75737 | fulllength | 5108 | NM_006197; pericentriolar material 1 |
| 6 | Hs.23263.1 | AA731693 | Hs.23263.1 | GenBank | Hs.23263 | | 23274 | |
| 7 | Hs.75737.0 | NM_006197.1 | g5453855 | RefSeq | Hs.75737 | fulllength | 5108 | NM_006197; pericentriolar material 1 |
| 8 | Hs.87280.0 | AA521424 | Hs.87280.0_RC | GenBank | Hs.87280 | fulllength | 124460 | NM_153337; hypothetical protein MGC35578 |
| 9 | Hs.128766.0 | NM_018152.1 | g8922543 | RefSeq | Hs.128766 | fulllength | 55184 | NM_018152; chromosome 20 open reading frame 12 |
| 10 | Hs.130714.0 | AF161441.1 | Hs.130714.0 | GenBank | Hs.130714 | | | |
| 11 | Hs.286218.2 | AF172398.2 | g13124448 | GenBank | Hs.12284 | fulllength | 50848 | NM_016946; F11 receptor isoform a precursor NM_144501; F11 receptor |
| | | | | | | | | isoform a precursor NM_144502; F11 receptor isoform b NM_144503; F11 |
| | | | | | | | | receptor isoform a precursor NM_144504; F11 receptor isoform a precursor |
| 12 | Hs.76394.0 | NM_004092.2 | g12707569 | RefSeq | Hs.76394 | fulllength | 1892 | NM_004092; mitochondrial short-chain enoyl-coenzyme A hydratase 1 |
| | | | | | | | | precursor |
| 13 | Hs.63668.0 | NM_003264.1 | g4507528 | RefSeq | Hs.63668 | fulllength | 7097 | NM_003264; toll-like receptor 2 |
| 14 | Hs.10842.0 | BF112006 | Hs.10842.0_RC | GenBank | Hs.10842 | fulllength | 5901 | NM_006325; ras-related nuclear protein |
| 15 | Hs.180296.0 | NM_007045.1 | g5901953 | RefSeq | Hs.180296 | fulllength | 11116 | NM_007045; FGFR1 oncogene partner |
| 16 | Hs.69560.0 | AI887983 | Hs.69560.0_RC | GenBank | Hs.355933 | | | |
| 17 | Hs.154387.0 | NM_014673.1 | g7661909 | RefSeq | Hs.154387 | fulllength | 9694 | NM_014673; KIAA0103 gene product |
| 18 | Hs.155489.0 | NM_006372.1 | g5453805 | RefSeq | Hs.373499 | fulllength | 10492 | NM_006372; NS1-associated protein 1 |
| 19 | g182182 | M21535.1 | g182182 | GenBank | Hs.45514 | fulllength | 2078 | NM_004449; v-ets erythroblastosis virus E26 oncogene like |
| 20 | Hs.150395.0 | AW269834 | Hs.150395.0 | GenBank | Hs.401726 | | | |
| 21 | Hs.79284.0 | NM_002402.1 | g4505154 | RefSeq | Hs.79284 | fulllength | 4232 | NM_002402; mesoderm specific transcript isoform a NM_177524; |
| | | | | | | | | mesoderm specific transcript isoform b NM_177525; mesoderm, specific |
| | | | | | | | | transcript isoform b |
| 22 | Hs.41587.3 | U63139.1 | g158805 | GenBank | Hs.41587 | fulllength | 10111 | NM_005732; RAD50 homolog isoform, 1 NM_133482; RAD50 homolog |
| | | | | | | | | isoform 2 |
| 23 | Hs.17109.0 | AL021786 | Hs.17109.0_RC | GenBank | Hs.17109 | fulllength | 9452 | NM_004867; integral membrane protein 2A |
| 24 | Hs.8841.0 | BE858787 | Hs.8841.0.A1 | GenBank | Hs.406670 | fulllength | 84513 | NM_032483; HTPAP protein |
| 25 | Hs.27410.2 | AK021850.1 | Hs.27410.2.S1 | GenBank | Hs.27410 | fulllength | 55893 | NM_018660; papillomavirus regulatory factor PRF-1 |
| 26 | Hs.326391.0 | AL545998 | Hs.326391.0.A1 | GenBank | Hs.375767 | fulllength | 254295 | NM_174933; hypothetical protein MGC16638 |
| 27 | Hs.154574.0 | AI952836 | Hs.154574.0_RC | GenBank | Hs.154574 | est | | |
| 28 | Hs.54580.0 | AF220034.1 | g12407398 | GenBank | Hs.54580 | fulllength | 81603 | NM_030912; tripartite motif-containing 8 |
| 29 | Hs.58879.0 | BF222867 | Hs.58879.0_RC | GenBank | Hs.58879 | est | | |
| 30 | Hs.288905.0 | AA129774 | Hs.288905.0 | GenBank | Hs.288905 | | | |
| 31 | Hs.194692.0 | NM_021100.1 | g10864078 | RefSeq | Hs.194692 | fulllength | 9054 | NM_021100; NFS1 nitrogen fixation 1 |
| 32 | g13625770 | AF276658.1 | g13625770 | GenBank | Hs.134707 | fulllength | 84557 | NM_032514; microtubule-associated protein 1 light chain 3 alpha isoform a |
| | | | | | | | | NM_181509; microtubule-associated protein 1 light chain 3 alpha isoform b |
| 33 | Hs.17109.0 | NM_004867.1 | g4758223 | RefSeq | Hs.17109 | fulllength | 9452 | NM_004867; integral membrane protein 2A |
| 34 | Hs.156114.0 | D86043.1 | g1864010 | GenBank | Hs.156114 | fulllength | 140885 | NM_004648; NM_080792; protein tyrosine phosphatase, non-receptor type |
| | | | | | | | | substrate 1 precursor |
| 35 | Hs.288791.0 | NM_013255.1 | g7019458 | RefSeq | Hs.288791 | fulllength | 4289 | NM_013255; muskelin 1, intracellular mediator containing kelch motifs |
| 36 | Hs.31854.0 | AB038651.1 | g9664093 | GenBank | Hs.31854 | fulllength | 57106 | NM_020378; K562 cell-derived leucine-zipper-like protein 1 |
| 37 | Hs.100651.2 | BF966558 | Hs.100651.2 | GenBank | Hs.100651 | fulllength | 9570 | NM_004287; golgi SNAP receptor complex member 2 isoform A |
| | | | | | | | | NM_054022; golgi SNAP receptor complex member 2 isoform B |
| 38 | Hs.239444.0 | AW003666 | Hs.239444.0.A1 | GenBank | Hs.239444 | est | | |
| 39 | Hs.74497.1 | BC002411.1 | g12803206 | GenBank | Hs.74497 | fulllength | 4904 | NM_004559; nuclease sensitive element binding protein 1 |
| 40 | Hs.287994.1 | AF125672.1 | Hs.287994.1_RC | GenBank | Hs.287994 | fulllength | 9612 | NM_006312; nuclear receptor co-repressor 2 |
| 41 | Hs.86978.0 | NM_002726.1 | g4506042 | RefSeq | Hs.86978 | fulllength | 5550 | NM_002726; prolyl endopeptidase |
| 42 | Hs.278672.0 | NM_005898.1 | g5174502 | RefSeq | Hs.278672 | fulllength | 4076 | NM_005898; membrane component, chromosome 11, surface marker 1 |
| 43 | Hs.8102.2 | AF113008.1 | Hs.8102.2_RC | GenBank | Hs.8102 | fulllength | 6224 | NM_001023; ribosomal protein S20 |
| 44 | Hs.77313.0 | NM_003674.1 | g4502730 | RefSeq | Hs.77313 | fulllength | 8558 | NM_003674; cyclin-dependent kinase 10 isoform 1 NM_052987; cyclin- |
| | | | | | | | | dependent kinase 10 isoform 2 NM_052988; cyclin-dependent kinase 10 |
| | | | | | | | | isoform 3 |
| 45 | Hs.1342.0 | NM_001862.1 | g4502982 | RefSeq | Hs.1342 | fulllength | 1329 | NM_001862; cytochrome c oxidase subunit Vb precursor |
| 46 | Hs.173694.0 | AB029020.1 | Hs.173694.0.S1 | GenBank | Hs.173694 | fulllength | 23032 | NM_015017; pVHL-interacting deubiquitinating enzyme 1 |
| 47 | Hs.179615.0 | NM_017985.1 | g8922211 | RefSeq | Hs.179615 | fulllength | 55064 | NM_017985; hypothetical protein FLJ10058 |
| 48 | Hs.181693.0 | AI732512 | Hs.181693.0.A1 | GenBank | Hs.445400 | est | | |
| 49 | Hs.111076.0 | BC001917.1 | g12804928 | GenBank | Hs.343521 | fulllength | 4191 | NM_005918; mitochondrial malate dehydrogenase precursor |
| 50 | Hs.130732.1 | AF317392.1 | g11907999 | GenBank | Hs.130732 | fulllength | 54880 | NM_017745; BCL-6 interacting corepressor isoform 1 NM_020926; BCL-6 |
| | | | | | | | | interacting corepressor isoform 2 |
| 51 | Hs.77500.1 | AF017306.1 | g2656142 | GenBank | Hs.77500 | fulllength | 7375 | NM_003363; ubiquitin specific protease, proto-oncogene |
| 52 | Hs.70186.0 | NM_003169.1 | g4507312 | RefSeq | Hs.70186 | fulllength | 6829 | NM_003169; suppressor of Ty 5 homolog |
| 53 | Hs.8562.0 | NM_017793.1 | g8923354 | RefSeq | Hs.8562 | fulllength | 54913 | NM_017793; hypothetical protein FLJ20374 |
| 54 | Hs.181369.1 | BC001049.1 | g12654446 | GenBank | Hs.199402 | fulllength | 7353 | NM_005659; ubiquitin fusion degradation 1-like |
| 55 | Hs.179972.0 | AU135021 | Hs.179972.0 | GenBank | Hs.179972 | fulllength | 55082 | NM_018011; hypothetical protein FLJ10154 |
| 56 | Hs.25252.0 | AA843963 | Hs.25252.0 | GenBank | Hs.25252 | | | |
| 57 | Hs.247985.0 | U72237 | Hs.247985.0 | GenBank | | | | |
| 58 | Hs.165433.0 | BG250498 | Hs.165433.0_RC | GenBank | Hs.165433 | | 83860 | |
| 59 | Hs.184877.1 | AF070548.1 | g3387910 | GenBank | Hs.184877 | fulllength | 8402 | NM_003562; solute carrier family 25 (mitochondrial carrier; oxoglutarate |
| | | | | | | | | carrier), member 11 |
| 60 | Hs.239458.0 | NM_021065.1 | Hs.239458.0 | GenBank | Hs.239458 | fulllength | 3013 | NM_021065; histone 1, H2ad |
| 61 | Hs.306079.1 | AI719730 | Hs.306079.1.A1 | GenBank | Hs.75295 | fulllength | 2982 | NM_000856; guanylate cyclase 1, soluble, alpha 3 |
| 62 | Hs.15395.0 | AK023550.1 | Hs.15395.0.S1 | GenBank | Hs.15395 | fulllength | 57038 | NM_020320; arginyl-tRNA synthetase-like |
| 63 | Hs.27996.0 | N34402 | Hs.27996.0.A1 | GenBank | Hs.27996 | est | | |
| 64 | Hs.79708.0 | AI860159 | Hs.79708.0_RC | GenBank | Hs.79708 | | | |
| 65 | Hs.282076.0 | BF439330 | Hs.282076.0 | GenBank | Hs.282076 | | 85358 | |
| 66 | Hs.99029.0 | AL564683 | Hs.99029.0 | GenBank | Hs.99029 | fulllength | 1051 | NM_005194; CCAAT/enhancer binding protein beta |
| 67 | Hs.164915.1 | U71300.1 | g1619945 | GenBank | Hs.164915 | fulllength | 6619 | NM_003084; small nuclear RNA activating complex, polypeptide 3, 50 kDa |
| 68 | Hs.285013.0 | BE560202 | Hs.285013.0.S2 | GenBank | Hs.285013 | est | 8125 | NM_006305; acidic (leucine-rich) nuclear phosphoprotein 32 family, |
| | | | | | | | | member A |
| 69 | Hs.21941.0 | AF260333.1 | g12005903 | GenBank | Hs.323583 | fulllength | 51313 | NM_016613; hypothetical protein DKFZp434L142 NM_032022; |
| 70 | Hs.107139.0 | NM_016619.1 | g7706157 | RefSeq | Hs.107139 | fulllength | 51316 | NM_016619; placenta-specific 8 |
| 71 | Hs.156114.0 | NM_004648.1 | g4758977 | RefSeq | Hs.156114 | fulllength | 140885 | NM_004648; NM_080792; protein tyrosine phosphatase, non-receptor type |
| | | | | | | | | substrate 1 precursor |
| 72 | Hs.211568.1 | BE966878 | Hs.211568.1_RC | GenBank | Hs.433750 | fulllength | 1981 | NM_004953; eukaryotic translation initiation factor 4 gamma, 1 |
| 73 | Hs.12461.0 | AF116707.1 | g7959912 | GenBank | Hs.233044 | fulllength | 57189 | |
| 74 | Hs.2340.0 | NM_021991.1 | g12056467 | RefSeq | Hs.2340 | fulllength | 3728 | NM_002230; junction plakoglobin NM_021991; junction plakoglobin |
| 75 | Hs.237642.1 | AI916528 | Hs.237642.1.A1 | GenBank | Hs.237642 | | | |
| 76 | Hs.227913.0 | NM_006595.1 | g5729729 | RefSeq | Hs.227913 | fulllength | 8539 | NM_006595; apoptosis inhibitor 5 NM_021112; |
| 77 | Hs.61712.1 | AU146532 | Hs.61712.1 | GenBank | Hs.61712 | fulllength | 5163 | NM_002610; pyruvate dahydrogenase kinase, isoenzyme 1 |
| 78 | Hs.124165.0 | AW272333 | Hs.124165.0_RC | GenBank | Hs.124165 | est | | |
| 79 | Hs.278904.0 | NM_015510.1 | g7661657 | RefSeq | Hs.11411 | fulllength | 25979 | NM_015510; DKFZP566O084 protein NM_016036; |
| 80 | Hs.27801.2 | AI807017 | Hs.27801.2 | GenBank | Hs.27801 | fulllength | 23598 | NM_014323; zinc finger protein 278 long C isoform NM_032050; zinc finger |
| | | | | | | | | protein 278 long A isoform NM_032051; zinc finger protein 278 short |
| | | | | | | | | isoform NM_032052; zinc finger protein 278 long B isoform |
| 81 | Hs.332138.0 | NM_030652.1 | g13449286 | RefSeq | Hs.332138 | fulllength | 80864 | NM_030652; NG3 protein |
| 82 | Hs.127924.0 | AI016855 | Hs.127924.0_RC | GenBank | Hs.303157 | fulllength | 6957 | NG_001333; |
| 83 | Hs.241543.0 | AW151250 | Hs.241543.0.S1 | GenBank | Hs.241543 | fulllength | 26073 | NM_015584; DNA polymerase delta p38 subunit |
| 84 | Hs.5437.3 | BF477658 | Hs.5437.3.A1 | GenBank | Hs.296261 | | | |
| 85 | Hs.12820.0 | NM_006590.1 | g5730024 | RefSeq | Hs.12820 | fulllength | 10713 | NM_006590; SnRNP assembly defective 1 homolog |
| 86 | Hs.202241.0 | AI697668 | Hs.202241.0_RC | GenBank | Hs.202241 | est | | |
| 87 | Hs.66170.1 | AF070592.1 | Hs.66170.1_RC | GenBank | Hs.66170 | fulllength | 56950 | NM_020197; SET and MYND domain containing 2 |
| 88 | Hs.760.1 | AL563460 | Hs.760.1 | GenBank | Hs.760 | fulllength | 84724 | |
| 89 | Hs.18271.0 | NM_022130.1 | g11545858 | RefSeq | Hs.18271 | fulllength | 64083 | NM_022130; golgi phosphoprotein 3 |
| 90 | Hs.81665.0 | NM_000222.1 | g4557694 | RefSeq | Hs.81665 | fulllength | 3815 | NM_000222; v-kit Hardy-Zuckerman 4 feline sarcoma viral oncogene |
| | | | | | | | | homolog precursor |
| 91 | Hs.191633.0 | BF223154 | Hs.191633.0_RC | GenBank | Hs.191633 | est | | |
| 92 | Hs.25674.2 | AI827820 | Hs.25674.2.S1 | GenBank | Hs.25674 | fulllength | 8932 | NM_003927; methyl-CpG binding domain protein 2 isoform 1 NM_015832; |
| | | | | | | | | methyl-CpG binding domain protein 2 testis-specific isoform |
| 93 | Hs.129837.0 | AW006067 | Hs.129837.0_RC | GenBank | Hs.129837 | fulllength | 127557 | NM_144621; hypothetical protein MGC17919 |
| 94 | Hs.302098.0 | AW139915 | Hs.302098.0.S1 | GenBank | Hs.437588 | est | | |
| 95 | Hs.154045.0 | D43949.1 | Hs.154045.0 | GenBank | Hs.154045 | fulllength | 23070 | NM_015050; KIAA0082 protein |
| 96 | Hs.91916.0 | AF035317.1 | Hs.91916.0 | GenBank | Hs.91916 | | | |
| 97 | Hs.34136.0 | NM_016940.1 | g8393017 | RefSeq | Hs.34136 | fulllength | 10069 | NM_016940; chromosome 21 open reading frame 6 |
| 98 | Hs.232400.0 | AI375753 | Hs.232400.0.S1 | GenBank | Hs.288649 | | | |
| 99 | Hs.131057.0 | AI742810 | Hs.131057.0_RC | GenBank | Hs.131057 | est | | |
| 100 | Hs.279477.0 | AI351043 | Hs.279477.0.A1 | GenBank | Hs.45514 | fulllength | 2078 | NM_004449; v-ets erythroblastosis virus E26 oncogene like |

TABLE 19
_all

| # | affy id | HUGO name | F | p | q | Title | MapLocation | Sequence Type | Transcript ID |
|---|---|---|---|---|---|---|---|---|---|
| 1 | 200923_at | LGALS3BP | 159.32 | 9.38e−32 | 3.30e−27 | lectin, galactoside-binding, soluble, 3 binding | 17q25 | Exemplarsequence | Hs.79339.0 |
| | | | | | | protein | | | |
| 2 | 200090_at- | FNTA | 50.56 | 8.32e−10 | 1.14e−05 | farnesyltransferase, CAAX box, alpha | 8p22-q11 | Consensussequence | Hs.138381.1 |
| | HG-U133B | | | | | | | | |
| 3 | 201433_s_at | PTDSS1 | 50.33 | 9.72e−10 | 1.14e−05 | phosphatidylserine synthase 1 | 8q22 | Exemplarsequence | Hs.77329.0 |
| 4 | 214937_x_at | PCM1 | 49.02 | 1.52e−09 | 1.30e−05 | pericentriolar material 1 | 8p22-p21.3 | Consensussequence | Hs.75737.2 |
| 5 | 200608_s_at | RAD21 | 46.59 | 2.06e−09 | 1.30e−05 | RAD21 homolog (S. pombe) | 8q24 | Exemplarsequence | Hs.81848.0 |
| 6 | 205849_s_at | UQCRB | 45.35 | 2.99e−09 | 1.30e−05 | ubiquinol-cytochrome c reductase binding protein | 8q22 | Exemplarsequence | Hs.131255.0 |
| 7 | 212250_at | | 45.30 | 3.44e−09 | 1.30e−05 | Homo sapiens, Similar to LYRIC, clone MGC: 41931 IMAGE: | | Consensussequence | Hs.243901.0 |
| | | | | | | 5298467, mRNA, complete cds | | | |
| 8 | 203534_at | LSM1 | 45.22 | 4.07e−09 | 1.30e−05 | LSM1 homolog, U6 small nuclear RNA associated (S. cerevisiae) | 8p11.2 | Exemplarsequence | Hs.111783.0 |
| 9 | 202174_s_at | PCM1 | 45.18 | 4.79e−09 | 1.30e−05 | pericentriolar material 1 | 8p22-p21.3 | Exemplarsequence | Hs.75737.0 |
| 10 | 225534_at | LOC114926 | 44.75 | 15.14e−09 | 1.30e−05 | hypothetical protein BC013035 | 8p11.1 | Consensussequence | Hs.10018.1 |
| 11 | 201754_at | COX6C | 44.51 | 5.18e−09 | 1.30e−05 | cytochrome c oxidase subunit VIc | 8q22-q23 | Exemplarsequence | Hs.74649.0 |
| 12 | 208845_at | | 44.14 | 5.5.4e−09 | 1.30e−05 | | | Exemplarsequence | Hs.7381.0 |
| 13 | 218482_at | DC6 | 43.68 | 7.08e−09 | 143e−05 | DC6 protein | 8q23.2 | Exemplarsequence | Hs.283740.0 |
| 14 | 200090_at- | FNTA | 42.83 | 8.58e−09 | 1.51e−05 | farnesyltransferase, CAAX box, alpha | 8p22-q11 | Consensussequence | Hs.138381.1 |
| | HG-U133A | | | | | | | | |
| 15 | 217819_at | LOC51125 | 42.35 | 9.39e−09 | 1.57e−05 | HSPC041 protein | 8p11.21 | Exemplarsequence | Hs.7953.0 |
| 16 | 237291_at | | 41.07 | 5.91e−09 | 1.30e−05 | ESTs, Weakly similar to unknown protein [Arabidopsis thaliana] | | Consensussequence | Hs.159362.0 |
| | | | | | | | [A. thaliana] | | |
| 17 | 228366_at | SID6-306 | 40.65 | 4.04e−09 | 1.30e−05 | inorganic pyrophosphatase 2 | 4q25 | Consensussequence | Hs.61455.0 |
| 18 | 203110_at | PTK2B | 40.01 | 1.52e−08 | 2.43e−05 | PTK2B protein tyrosine kinase 2 beta | 8p21.1 | Exemplarsequence | Hs.20313.0 |
| 19 | 225233_at | | 39.12 | 2.49e−09 | 1.30e−05 | Homo sapiens, clone IMAGE: 4154313, mRNA, partial cds | | Consensussequence | Hs.42179.0 |
| 20 | 226453_at | FLJ20974 | 38.04 | 4.43e−09 | 1.30e−05 | hypothetical protein FLJ20974 | 11q13.1 | Consensussequence | Hs.6364.3 |
| 21 | 214118_x_at | PCM1 | 36.86 | 5.16e−08 | 6.96e−05 | pericentriolar material 1 | 8p22-p21.3 | Consensussequence | Hs.75737.1 |
| 22 | 212248_at | | 36.32 | 7.65e−08 | 9.59e−05 | Homo sapiens. Similar to LYRIC, clone MGC: 41931 IMAGE: 5298467, | | Consensussequence | Hs.243901.0 |
| | | | | | | mRNA, complete cds | | | |
| 23 | 214089_at | RPS8 | 35.94 | 7.30e−09 | 1.43e−05 | ribosomal protein S8 | 1p34.1-p32 | Consensussequence | Hs.227400.1 |
| 24 | 209096_at | UBE2V2 | 35.66 | 9.50e−08 | 1.11e−04 | ubiquitin-conjugating enzyme E2 variant 2 | 8q11.1 | Exemplarsequence | Hs.79300.0 |
| 25 | 242434_at | | 35.60 | 3.12e−08 | 4.57e−05 | Homo sapiens cDNA FLJ31093 fis, clone | | Consansussequence | Hs.191581.0 |
| | | | | | | IMR321000161. | | | |
| 26 | 237108_x_at | DKFZp761G0122 | 35.38 | 8.34e−09 | 1.51e−05 | hypothetical protein DKFZp761G0122 | 1p36.32 | Consensussequence | Hs.198661.0 |
| 27 | 208647_at | FDFT1 | 34.82 | 1.20e−07 | 1.31e−04 | farnesyl-diphosphate famesyltransferase 1 | 8p23.1-p22 | Consensussequence | Hs.48876.1 |
| 28 | 32541_at | PPP3CC | 34.43 | 1.35e−07 | 1.36e−04 | protein phosphatase 3 (formerly 2B). catalytic | 8p21.2 | Consensussequence | 6 |
| | | | | | | subunit, gamma isoform (calcineurin A gamma) | | | |
| 29 | 207000_s_at | PPP3CC | 34.24 | 1.47e−07 | 1.40e−04 | protein phosphatase 3 (formerly 2B), catalytic | 8p21.2 | Exemplarsequence | Hs.75206.0 |
| | | | | | | subunit, gamma isoform (calcineurin A gamma) | | | |
| 30 | 225240_s_at | | 34.03 | 4.56e−08 | 6.40e−05 | Homo sapiens, clone IMAGE: 4154313, mRNA, partial cds | | Consensussequence | Hs.42179.0 |
| 31 | 218125_s_at | FLJ10853 | 33.69 | 1.87e−07 | 1.73e−04 | hypothetical protein FLJ10853 | 8p21.1 | Exemplarsequence | Hs.72085.0 |
| 32 | 202824_s_at | TCEB1 | 33.30 | 2.00e−07 | 1.80e−04 | transcription elongation factor B (SIII), polypeptide | 6q13.3 | Exemplarsequence | Hs.184693.0 |
| | | | | | | 1 (15 kDa, elongin C) | | | |
| 33 | 201652_at | COPS5 | 32.89 | 2.20e−07 | 1.86e−04 | COP9 constitutive photomorphogenic homolog | 8q12.3 | Exemplarsequence | Hs.198767.0 |
| | | | | | | subunit 5 (Arabidopsis) | | | |
| 34 | 204278_s_at | EBAG9 | 32.36 | 3.24e−07 | 12.53e−04 | estrogen receptor binding site associated, antigen, 9 | 8q23 | Exemplarsequence | Hs.9222.0 |
| 35 | 218747_s_at | TAPBP-R | 32.34 | 1.23e−07 | 1.31e−04 | TAP binding protein related | 12p13.31 | Exemplarsequence | Hs.267993.0 |
| 36 | 204341_at | TRIM16 | 32.13 | 3.06e−08 | 4.57e−05 | tripartite motif-containing 16 | 17p11.2 | Exemplarsequence | Hs.241305.0 |
| 37 | 243579_at | MSI2 | 32.04 | 9.52e−08 | 1.11e−04 | musashi homolog 2 (Drosophila) | 17q23.1 | Consensussequence | Hs.173179.0 |
| 38 | 229949_at | | 31.88 | 1.19e−07 | 1.31e−04 | Homo sapiens cDNA FLJ33372 fis, clone | | Consensussequence | Hs.325158.0 |
| | | | | | | BRACE2005981. | | | |
| 39 | 232989_s_at | DKFZp434G156 | 31.63 | 5.84e−08 | 7.60e−05 | hypothetical protein DKFZp434G156 | 7q33 | Consensussequence | Hs.7973.2 |
| 40 | 202634_at | POLR2K | 31.62 | 3.35e−07 | 2.56e−04 | polymerase (RNA) II (DNA directed) polypeptide | 8q22.2 | Consensussequence | Hs.150675.0 |
| | | | | | | K, 7.0 kDa | | | |
| 41 | 209471_s_at | FNTA | 31.32 | 4.25e−07 | 2.92e−04 | farnesyltransfarase, CAAX box, alpha | 8p22-q11 | Exemplarsequence | Hs.138381.0 |
| 42 | 227943_at | | 30.97 | 1.42e−07 | 1.39e−04 | ESTs | | Consensussequence | Hs.25933.0 |
| 43 | 225676_s_at | DKFZP564O0463 | 30.83 | 4.57e−07 | 3.03e−04 | DKFZP564O0463 protein | 8q22.3 | Consensussequence | Hs.273344.1 |
| 44 | 226942_at | FLJ21615 | 30.69 | 4.97e−07 | 3.24e−04 | hypothetical protein FLJ21615 | 8q24.22 | Consensussequence | Hs.44159.0 |
| 45 | 224836_at | C20orf110 | 30.47 | 1.30e−07 | 1.34e−04 | chromosome 20 open reading frame 110 | 20q11.22 | Consensussequence | Hs.11114.0 |
| 46 | 201418_s_at | SOX4 | 30.23 | 2.19e−07 | 1.86e−04 | SRY (sex determining region Y)-box 4 | 6p22.3 | Exemplarsequence | Hs.83484.0 |
| 47 | 214394_x_at | EEF1D | 29.84 | 5.95e−07 | 3.80e−04 | eukaryotic translation elongation factor 1 delta | 8q24.3 | Consensussequence | Hs.223241.1 |
| | | | | | | (guanine nucleotide exchange protein) | | | |
| 48 | 213204_at | KIAA0708 | 29.59 | 4.13e−07 | 2.92e−04 | KIAA0708 protein | 6p21.1 | Consensussequence | Hs.117177.0 |
| 49 | 213110_s_at | COL4A5 | 29.53 | 2.62e−07 | 2.14e−04 | collagen, type IV, alpha 5 (Alport syndrome) | Xq22 | Consensussequence | Hs.169825.0 |
| 50 | 203790_s_at | UK114 | 29.51 | 8.76e−07 | 5.40e−04 | translational inhibitor protein p14.5 | 8q22 | Consensussequence | Hs.18426.0 |

| | Sequence | | Sequence | | | | |
|---|---|---|---|---|---|---|---|
| # | Derived From | Sequence ID | Source | Unigene_Accession | Cluster_Type | LocusLink | Full_Length_Reference_Seq |
| 1 | NM_005567.2 | g6006016 | RefSeq | Hs.79339 | fulllength | 3959 | NM_005567; galectin 3 binding protein |
| 2 | BG168896 | Hs.138381.1A1 | GenBank | Hs.356463 | fulllength | 2339 | NM_002027; famesyltransferase, CAAX box, alpha |
| 3 | NM_014754.1 | g7662646 | RefSeq | Hs.77329 | fulllength | 9791 | NM_014754; phosphatidylserine synthase 1 |
| 4 | AI924817 | Hs.75737.2.S1 | GenBank | Hs.75737 | fulllength | 5108 | NM_006197; pericentriolar material 1 |
| 5 | NM_006265.1 | g5453993 | RefSeq | Hs.81848 | fulllength | 5885 | NM_006265; RAD21 homolog |
| 6 | NM_006294.1 | g5454151 | RefSeq | Hs.131255 | fulllength | 7381 | NM_006294; ubiquinol-cytochrome c reductase binding protein |
| 7 | AI972475 | Hs.243901.0.S1 | GenBank | Hs.243901 | fulllength | | |
| 8 | NM_014462.1 | g7657312 | RefSeq | Hs.425311 | fulllength | 27257 | NM014462; Lsm1 protein |
| 9 | NM_006197.1 | g5453855 | RefSeq | Hs.75737 | fulllength | 5108 | NM_006197; pericentriolar material 1 |
| 10 | AV711345 | Hs.10018.1.A1 | GenBank | Hs.10018 | fulllength | 114926 | NM_138436; hypothetical protein BC013035 |
| 11 | NM_004374.1 | g4758039 | RefSeq | Hs.351875 | fulllength | 1345 | NM_004374; cytochrome c oxidase subunit VIc proprotein |
| 12 | BC002456.1 | g12803280 | GenBank | | | | |
| 13 | NM_020189.1 | g9910185 | RefSeq | Hs.283740 | fulllength | 56943 | NM_020189; DC6 protein |
| 14 | BG168896 | Hs.138381.1.A1 | GenBank | Hs.358463 | fulllength | 2339 | NM_002027; farnesyltransferase, CAAX box, alpha |
| 15 | NM_016099.1 | g7705820 | RefSeq | Hs.7953 | fulllength | 51125 | NM_016099; HSPC041 protein |
| 16 | AI695007 | Hs.159362.0.A1 | GenBank | Hs.159362 | est | | |
| 17 | BF446912 | Hs.61455.0.A1 | GenBank | Hs.5123 | fulllength | 27068 | NM_006903; inorganic pyrophosphatase 2 isoform 2 |
| | | | | | | | NM_176865; inorganic pyrophosphatase 2 isoform 1 |
| | | | | | | | NM_176866; inorganic pyrophosphatase 2 isoform 3 |
| | | | | | | | NM_176867; inorganic pyrophosphatase 2 isoform 4 |
| | | | | | | | NM_176869; inorganic pyrophosphatase 2 isoform 1 |
| 18 | U43522.1 | g1165218 | GenBank | Hs.20313 | fulllength | 2185 | NM_004103; PTK2B protein tyrosine kinase 2 beta isoform a NM_173174; |
| | | | | | | | PTK2B protein tyrosine kinase 2 beta isoform a NM_173175; |
| | | | | | | | PTK2B protein tyrosine kinase 2 beta isoform b NM_173176; |
| | | | | | | | PTK2B protein tyrosine kinase 2 beta isoform a |
| 19 | BF435123 | Hs.42179.0.A1 | GenBank | Hs.173179 | | | |
| 20 | BF982002 | Hs.6364.3.A1 | GenBank | Hs.334800 | fulllength | 84153 | NM_032193; hypothetical protein FLJ20974 |
| 21 | AI205598 | Hs.75737.1.S1 | GenBank | Hs.75737 | fulllength | 5108 | NM_006197; pericentriolar material 1 |
| 22 | AI972475 | Hs.243901.0.S1 | GenBank | Hs.243901 | fulllength | | |
| 23 | AA976278 | Hs.227400.1_RC | GenBank | Hs.399720 | fulllength | 6202 | NM_001012; ribosomal protein S8 |
| 24 | U62136.2 | g4775663 | GenBank | Hs.79300 | fulllength | 7336 | NM_003350; ubiquitin-conjugating enzyme E2 variant 2 |
| 25 | AW771952 | Hs.191581.0_RC | GenBank | Hs.191581 | | | |
| 26 | AW611845 | Hs.198661.0_RC | GenBank | Hs.6600 | fulllength | 199986 | NM_152661; hypothetical protein DKFZp761G0122 |
| 27 | AA872727 | Hs.48876.1 | GenBank | Hs.48876 | fulllength | 2222 | NM_004462; farnesyl-diphosphate farnesyltransferase 1 |
| 28 | S46622 | 4922761 | GenBank | Hs.75206 | fulllength | 5533 | NM_005605; protein phosphatase 3 (formerly 2B), catalytic subunit, gamma isoform (calcineurin |
| | | | | | | | A gamma) |
| 29 | NM_005605.1 | g5031988 | RefSeq | Hs.75206 | fulllength | 5533 | NM_005605; protein phosphatase 3 (formerly 2B), catalytic subunit, gamma isoform (calcineurin A |
| | | | | | | | gamma) |
| 30 | BF435123 | Hs.42179.0.A1 | GenBank | Hs.173179 | | | |
| 31 | NM_018246.1 | g8922717 | RefSeq | Hs.72085 | fulllength | 55246 | NM_018246; hypothetical protein FLJ10853 |
| 32 | NM_005648.1 | g5032160 | RefSeq | Hs.184693 | fulllength | 6921 | NM_005648; elongin C |
| 33 | NM_006837.1 | g5803045 | RefSeq | Hs.380969 | fulllenglh | 10987 | NM_006837; COP9 constitutive photomorphogenic homolog subunit 5 |
| 34 | NM_004215.1 | g4758229 | RefSeq | Hs.9222 | fulllength | 9166 | NM_004215; estrogen receptor binding site associated antigen 9 |
| 35 | NM_018009.1 | g8922253 | RefSeq | Hs.267993 | fulllength | 55080 | NM_018009; TAP binding protein related |
| 36 | NM_006470.1 | g5453643 | RefSeq | Hs.241305 | fulllength | 10626 | NM_006470; tripartite motif-containing 16 |
| 37 | BF029215 | Hs.173179.0.S1 | GenBank | Hs.103512 | fulllength | 124540 | NM_138962; musashi 2 isoform a NM_170721; musashi 2 isoform b |
| 38 | AA554827 | Hs.325158.0_RC | GenBank | Hs.370705 | | | |
| 39 | AK023500.1 | Hs.7973.2 | GenBank | Hs.7973 | fulllength | 64753 | NM_022742; hypothetical protein DKFZp434G156 |
| 40 | AL558030 | Hs.150675.0 | GenBank | Hs.351475 | fulllength | 5440 | NM_005034; DNA directed RNA polymerase II polypeptide K |
| 41 | L00634.1 | g292030 | GenBank | Hs.356463 | fulllength | 2339 | NM_002027; farnesyltransferase, CAAX box, alpha |
| 42 | AI798680 | Hs.25933.0_RC | GenBank | Hs.445018 | est | | |
| 43 | AK001693.1 | Hs.273344.1 | GenBank | Hs.273344 | fulllength | 25879 | NM_014156; NM_015420; DKFZP564O0463 protein |
| 44 | AI742668 | Hs.44159.0.S1 | GenBank | Hs.44159 | fulllength | 84165 | NM_032205; hypothetical protein FLJ21615 |
| 45 | AL109824 | Hs.11114.0.A1 | GenBank | Hs.11114 | fulllength | 58476 | |
| 46 | NM_003107.1 | g4507162 | RefSeq | Hs.83484 | fulllength | 6659 | NM_003107; SRY (sex determining region Y)-box 4 |
| 47 | AI613383 | Hs.223241.1.A1 | GenBank | Hs.334798 | fulllength | 1936 | NM_001960; eukaryotic translation elongation factor 1 delta isoform 2 NM_032378; eukaryotic |
| | | | | | | | translation elongation factor 1 delta isoform 1 |
| 48 | AB014608.1 | Hs.117177.0 | GenBank | Hs.117177 | fulllength | 23113 | NM_015089; p53-associated parkin-like cytoplasmic protein |
| 49 | AW052179 | HS.169825.0_RC | GenBank | Hs.169825 | fulllength | 1287 | NM_000495; alpha 5 type IV collagen isoform 1, precursor |
| | | | | | | | NM_033380; alpha 5 type IV collagen isoform 2, precursor |
| | | | | | | | NM_033381; alpha 5 type IV collagen isoform 3, precursor |
| 50 | N54448 | Hs.18426.0.S1 | GenBank | Hs.18426 | fulllength | 10247 | NM_005836; translational inhibitor protein p14.5 |

TABLE 20
_ALL

| | | | | | | | Sequence |
|---|---|---|---|---|---|---|---|
| # | affy id | HUGO name | Title | MapLocation | Sequence Type | Transcript ID | Derived From |
| 1 | 201398_s_at | TRAM | translocating chain-associating membrane | 8q13.1 | Exemplarsequence | Hs.4147.0 | BC000687.1 |
| | | | protein | | | | |
| 2 | 203208_s_at | CHPPR | likely ortholog of chicken chondrocyte | 8q12.1 | Exemplarsequence | Hs.170198.0 | NM_014637.1 |
| | | | protein with a poly-protine region | | | | |
| 3 | 235509_at | MGC40214 | hypothetical protein MGC40214 | 8q22.1 | Consensussequence | Hs.98471.0 | AV662196 |

| | | Sequence | | | | |
|---|---|---|---|---|---|---|
| # | Sequence ID | Source | Unigene_Accession | Cluster_Type | LocusLink | Full_Length_Reference_Seq |
| 1 | g12653796 | GenBank | Hs.4147 | fulllength | 23471 | NM_014294; translocating chain- |
| | | | | | | associating membrane protein |
| 2 | g7661853 | RefSeq | Hs.170198 | fulllength | 9650 | NM_014637; KIAA0009 gene |
| | | | | | | product |
| 3 | Hs.98471.0_RC | GenBank | Hs.98471 | est | 137682 | NM_152416; hypothetical protein |
| | | | | | | MGC40214 |

TABLE 21
_t(8; 21)

| # | affy id | HUGO name | F | p | q | Title |
|---|---|---|---|---|---|---|
| 1 | 218273_s_at | PDP | 181.63 | 9.58e−16 | 4.30e−11 | pyruvate dehydrogenase phosphatase |
| 2 | 220572_at | DKFZp547G183 | 112.74 | 8.42e−13 | 1.89e−08 | hypothetical protein DKFZp547G183 |
| 3 | 219433_at | BCoR | 106.17 | 1.54e−12 | 2.30e−08 | BCL-6 interacting corepressor |
| 4 | 202126_at | PRPF4B | 92.11 | 1.61e−09 | 5.88e−06 | PRP4 pre-mRNA processing factor 4 |
| | | | | | | homolog B (yeast) |
| 5 | 230541_at | LOC149134 | 81.98 | 5.38e−11 | 6.04e−07 | hypothetical protein LOC149134 |
| 6 | 201562_s_at | SORD | 81.30 | 1.51e−10 | 1.36e−06 | sorbitol dehydrogenase |
| 7 | 208883_at | DD5 | 80.56 | 2.10e−03 | 6.97e−02 | progestin induced protein |
| 8 | 228188_at | FLJ23306 | 79.92 | 4.38e−04 | 2.42e−02 | hypothetical protein FLJ23306 |
| 9 | 225102_at | LOC152009 | 70.05 | 1.29e−09 | 5.80e−06 | hypothetical protein LOC152009 |
| 10 | 211622_s_at | ARF3 | 67.82 | 3.23e−09 | 9.67e−06 | ADP-ribosylation factor 3 |
| 11 | 200051_at- | SART1 | 67.41 | 6.47e−10 | 4.84e−06 | squamous cell carcinoma antigen |
| | HG-U133A | | | | | recognised by T cells |
| 12 | 203441_s_at | CDH2 | 66.13 | 8.62e−10 | 4.84e−06 | cadherin 2, type 1, N-cadherin (neuronal) |
| 13 | 239397_at | | 66.03 | 5.46e−04 | 2.82e−02 | Homo sapiens cDNA FLJ36260 fis, clone |
| | | | | | | THYMU2002548. |
| 14 | 204811_s_at | CACNA2D2 | 65.76 | 8.22e−10 | 4.84e−06 | calcium channel, voltage-dependent, alpha |
| | | | | | | 2/delta subunit 2 |
| 15 | 224019_at | FLJ21032 | 65.17 | 1.01e−08 | 2.01e−05 | hypothetical protein FLJ21032 |
| 16 | 210465_s_at | SNAPC3 | 64.38 | 1.09e−09 | 5.45e−06 | small nuclear RNA activating complex, |
| | | | | | | polypeptide 3, 50 kDa |
| 17 | 232647_at | MGC39650 | 64.25 | 1.61e−09 | 5.88e−06 | hypothetical protein MGC39650 |
| 18 | 240178_at | | 64.24 | 3.92e−06 | 1.12e−03 | ESTs |
| 19 | 218704_at | FLJ20315 | 63.98 | 8.61e−08 | 1.14e−04 | hypothetical protein FLJ20315 |
| 20 | 204198_s_at | RUNX3 | 62.56 | 1.32e−04 | 1.12e−02 | runt-related transcription factor 3 |
| 21 | 203756_at | P164RHOGEF | 61.93 | 1.70e−09 | 5.88e−06 | Rho-specific guanine-nucleotide exchange |
| | | | | | | factor 164 kDa |
| 22 | 207866_at | BMP8 | 60.30 | 2.58e−09 | 8.27e−06 | bone morphogenetic protein 8 (osteogenic |
| | | | | | | protein 2) |
| 23 | 213301_x_at | TIF1 | 59.60 | 2.85e−08 | 4.41e−05 | transcriptional intermediary factor 1 |
| 24 | 224855_at | P5CR2 | 59.13 | 1.19e−08 | 2.23e−05 | pyrroline 5-carboxylate reductase isoform |
| 25 | 213151_s_at | CDC10 | 58.86 | 1.77e−04 | 1.35e−02 | CDC10 cell division cycle 10 |
| | | | | | | homolog (S. cerevisiae) |
| 26 | 228225_at | PXMP3 | 58.64 | 4.06e−04 | 2.32e−02 | peroxisomal membrane protein 3, 35 kDa |
| | | | | | | (Zellweger syndrome) |
| 27 | 233955_x_at | HSPC195 | 58.61 | 1.78e−03 | 6.23e−02 | hypothetical protein HSPC195 |
| 28 | 213070_at | | 58.02 | 4.65e−07 | 3.12e−04 | Homo sapiens mRNA; cDNA |
| | | | | | | DKFZp564L222 (from clone |
| | | | | | | DKFZp564L222) |
| 29 | 224975_at | NFIA | 57.97 | 4.57e−09 | 1.28e−05 | nuclear factor I/A |
| 30 | 236530_at | | 57.20 | 2.19e−04 | 1.53e−02 | ESTs |
| 31 | 210935_s_at | WDR1 | 56.83 | 5.06e−09 | 1.34e−05 | WD repeat domain 1 |
| 32 | 219461_at | PAK6 | 56.43 | 2.53e−05 | 3.57e−03 | p21(CDKN1A)-activated kinase 6 |
| 33 | 236035_at | | 56.38 | 6.45e−09 | 1.52e−05 | ESTs |
| 34 | 219075_at | MGC3262 | 55.31 | 6.38e−09 | 1.52e−05 | hypothetical protein MGC3262 |
| 35 | 237839_at | | 54.59 | 7.47e−09 | 1.68e−05 | ESTs |
| 36 | 217699_at | | 54.59 | 2.05e−08 | 3.57e−05 | ESTs, Weakly similar to cytokine receptor- |
| | | | | | | like factor 2; cytokine receptor CRL2 |
| | | | | | | precusor [Homo sapiens] [H. sapiens] |
| 37 | 222461_s_at | HERC2 | 53.61 | 9.15e−09 | 1.96e−05 | hect domain and RLD 2 |
| 38 | 230093_at | TSGA2 | 53.37 | 1.03e−08 | 2.01e−05 | testes specific A2 homolog (mouse) |
| 39 | 221502_at | KPNA3 | 53.21 | 5.79e−04 | 2.92e−02 | karyopherin alpha 3 (importin alpha 4) |
| 40 | 205977_s_at | EPHA1 | 52.63 | 10.00e−08 | 1.18e−04 | EphA1 |
| 41 | 205054_at | NEB | 52.36 | 2.73e−05 | 3.74e−03 | nebulin |
| 42 | 236777_at | LOC221584 | 51.92 | 1.96e−06 | 6.88e−04 | hypothetical protein LOC221584 |
| 43 | 223158_s_at | NEK6 | 51.12 | 1.81e−06 | 6.66e−04 | NIMA (never in mitosis gene a)-related |
| | | | | | | kinase 6 |
| 44 | 205240_at | LGN | 50.96 | 2.07e−08 | 3.57e−05 | LGN protein |
| 45 | 214857_at | | 49.60 | 1.72e−04 | 1.33e−02 | Homo sapiens mRNA; cDNA |
| | | | | | | DKFZp566H0124 (from clone |
| | | | | | | DKFZp566H0124) |
| 46 | 218199_s_at | NOL6 | 49.56 | 6.07e−06 | 1.44e−03 | nucleolar protein family 6 (RNA-associated) |
| 47 | 218620_s_at | HEMK | 49.50 | 8.03e−08 | 1.13e−04 | HEMK homolog 7 kb |
| 48 | 221837_at | FLJ14360 | 49.50 | 2.22e−08 | 3.70e−05 | hypothetical protein FLJ14360 |
| 49 | 217566_s_at | TGM4 | 49.34 | 3.68e−07 | 2.71e−04 | transglutaminase 4 (prostate) |
| 50 | 227152_at | FLJ20696 | 49.23 | 4.39e−04 | 2.42e−02 | hypothetical protein FLJ20696 |

| # | MapLocation | Sequence Type | Transcript ID |
|---|---|---|---|
| 1 | 8q21.3 | Exemplarsequence | Hs.22265.0 |
| 2 | 11q13.5 | Exemplarsequence | Hs.283631.0 |
| 3 | Xp11.4 | Exemplarsequence | Hs.278732.0 |
| 4 | 6p25.1 | Consensussequence | Hs.198891.0 |
| 5 | 1q44 | Consensussequence | Hs.61829.0 |
| 6 | 15q15.3 | Exemplarsequence | Hs.878.0 |
| 7 | 8q22 | Consensussequence | Hs.278428.0 |
| 8 | 2p23.3 | Consensussequence | Hs.5890.0 |
| 9 | 3q21.3 | Consensussequence | Hs.3964.0 |
| 10 | 12q13 | Exemplarsequence | g178980 |
| 11 | 11q13.1 | Exemplarsequence | Hs.288319.0 |
| 12 | 18q11.2 | Exemplarsequence | Hs.161.0 |
| 13 | | Consensussequence | Hs.202402.0 |
| 14 | 3p21.3 | Exemplarsequence | Hs.127436.0 |
| 15 | 4q21.22 | Exemplarsequence | Hs.247474.1 |
| 16 | 9p22.2 | Exemplarsequence | Hs.164915.1 |
| 17 | 17q11.1 | Consensussequence | Hs.258890.0 |
| 18 | | Consensussequence | Hs.9451.0 |
| 19 | 17q23.2 | Exemplarsequence | Hs.18457.0 |
| 20 | 1p36 | Consensusequence | Hs.170019.0 |
| 21 | 11q13.2 | Exemplarsequence | Hs.45180.0 |
| 22 | 1p35-p32 | Exemplarsequence | Hs.99948.0 |
| 23 | 7q32-q34 | Consensussequence | Hs.183858.1 |
| 24 | 1q42.13 | Consensussequence | Hs.274287.0 |
| 25 | 7p14.3-p14.1 | Consensussequence | Hs.184326.1 |
| 26 | 8q21.1 | Consensussequence | Hs.180612.2 |
| 27 | 5q31.3 | Consensussequence | Hs.15093.1 |
| 28 | | Consensussequence | Hs.5848.0 |
| 29 | 1p31.3-p31.2 | Consensussequence | Hs.173933.0 |
| 30 | | Consensussequence | Hs.288410.0 |
| 31 | 4p16.1 | Exemplarsequence | Hs.326791.0 |
| 32 | 15q14 | Consensussequence | Hs.21420.0 |
| 33 | | Consensussequence | Hs.65788.0 |
| 34 | 19p13.13 | Exemplarsequence | Hs.323213.0 |
| 35 | | Consensussequence | Hs.233301.0 |
| 36 | | Consensussequence | Hs.286243.0 |
| 37 | 15q13 | Consensussequence | Hs.266933.0 |
| 38 | 21q22.3 | Consensussequence | Hs.7369.0 |
| 39 | 13q14.3 | Consensussequence | Hs.3886.0 |
| 40 | 7q32-q36 | Exemplarsequence | Hs.89839.0 |
| 41 | 2q22 | Exemplarsequence | Hs.83870.0 |
| 42 | 6p21.33 | Consensussequence | Hs.124603.0 |
| 43 | 9q33.3-q34.11 | Consensussequence | Hs.9625.2 |
| 44 | 1p13.2 | Exemplarsequence | Hs.278338.0 |
| 45 | | Consensussequence | Hs.133130.1 |
| 46 | 9p13.2 | Exemplarsequence | Hs.183253.0 |
| 47 | 3p21.3 | Exemplarsequence | Hs.46907.0 |
| 48 | 22q11.21 | Consensussequence | Hs.181341.1 |
| 49 | 3p22-p21.33 | Consensussequence | Hs.289803.0 |
| 50 | | Consensussequence | Hs.323822.0 |

| | Sequence | | Sequence | | | |
|---|---|---|---|---|---|---|
| # | Derived From | Sequence ID | Source | Unigene_Accession | Cluster_Type | LocusLink |
| 1 | NM_018444.1 | g8923959 | RefSeq | Hs.22265 | fulllength | 54704 |
| 2 | NM_018705.1 | g8922152 | RefSeq | Hs.283631 | fulllength | 55525 |
| 3 | NM_017745.1 | g8923266 | RefSeq | Hs.130732 | fulllength | 54880 |
| 4 | AA156948 | Hs.198891.0.S2 | GenBank | Hs.198891 | fulllength | 8899 |
| 5 | AU150080 | Hs.61829.0 | GenBank | Hs.417101 | | 149134 |
| 6 | NM_003104.1 | g4507154 | RefSeq | Hs.876 | fulllength | 6652 |
| 7 | U69567 | Hs.278428.0.A2 | GenBank | Hs.278428 | fulllength | 51366 |
| 8 | AI860150 | Hs.5890.0_RC | GenBank | Hs.5890 | fulllength | 79579 |
| 9 | BG168471 | Hs.3964.0 | GenBank | Hs.3964 | | 152009 |
| 10 | M33384.1 | g178980 | GenBank | Hs.119177 | fulllength | 377 |
| 11 | NM_005146.1 | g10863888 | RefSeq | Hs.288319 | fulllength | 9092 |
| 12 | NM_001792.1 | g4502720 | RefSeq | Hs.161 | fulllength | 1000 |
| 13 | AW390251 | Hs.202402.0_RC | GenBank | Hs.202402 | | |
| 14 | NM_006030.1 | g5174402 | RefSeq | Hs.127436 | fulllength | 9254 |
| 15 | BC004936.1 | g13436280 | GenBank | Hs.379191 | fulllength | 79966 |
| 16 | U71300.1 | g1619945 | GenBank | Hs.164915 | fulllength | 6619 |
| 17 | AL137531.1 | Hs.258890.0 | GenBank | Hs.207471 | fulllength | 147011 |
| 18 | H63394 | Hs.9451.0.A1 | GenBank | Hs.334792 | est | |
| 19 | NM_017763.1 | g8923298 | RefSeq | Hs.18457 | fulllength | 54894 |
| 20 | AA541630 | Hs.170019.0.S1 | GenBank | Hs.170019 | fulllength | 864 |
| 21 | NM_014786.1 | g7662063 | RefSeq | Hs.45180 | fulllength | 9828 |
| 22 | NM_001720.1 | g4502428 | RefSeq | Hs.99948 | fulllength | 656 |
| 23 | AL538264 | Hs.183858.1 | GenBank | Hs.183858 | fulllength | 8805 |
| 24 | AL561868 | Hs.274287.0 | GenBank | Hs.274287 | fulllength | 29920 |
| 25 | AU157515 | Hs.184326.1.S1 | GenBank | Hs.184326 | fulllength | 989 |
| 26 | AW512586 | Hs.180612.2.A1 | GenBank | Hs.180612 | fulllength | 5828 |
| 27 | AK001782.1 | Hs.15093.1 | GenBank | Hs.15093 | fulllength | 51523 |
| 28 | AV682436 | Hs.5848.0.S1 | GenBank | Hs.336446 | | |
| 29 | AK024964.1 | Hs.173933.0 | GenBank | Hs.173933 | fulllength | 4774 |
| 30 | BF589826 | Hs.288410.0_RC | GenBank | Hs.288410 | est | |
| 31 | AF274954.1 | g12751070 | GenBank | Hs.85100 | fulllength | 9948 |
| 32 | AJ236915.1 | Hs.21420.0 | GenBank | Hs.21420 | fulllength | 56924 |
| 33 | AW190406 | Hs.65788.0.A1 | GenBank | Hs.435027 | est | |
| 34 | NM_024029.1 | g13128975 | RefSeq | Hs.323213 | fulllength | 78992 |
| 35 | BF433975 | Hs.233301.0.A1 | GenBank | Hs.436623 | est | |
| 36 | AV700338 | Hs.286243.0 | GenBank | Hs.286243 | est | |
| 37 | BE671173 | Hs.266933.0 | GenBank | Hs.266933 | fulllength | 8924 |
| 38 | AI683428 | Hs.7369.0.A1 | GenBank | Hs.7369 | fulllength | 89765 |
| 39 | AL120704 | Hs.3886.0.A2 | GenBank | Hs.3886 | fulllength | 3839 |
| 40 | NM_005232.1 | g4885208 | RefSeq | Hs.89839 | fulllength | 2041 |
| 41 | NM_004543.2 | g8400716 | RefSeq | Hs.83870 | | 4703 |
| 42 | AA854843 | Hs.124603.0_RC | GenBank | Hs.124603 | | 221584 |
| 43 | BE616825 | Hs.9625.2.S1 | GenBank | Hs.9625 | fulllength | 10783 |
| 44 | NM_013296.1 | g9558734 | RefSeq | Hs.278338 | fulllength | 29899 |
| 45 | AL050035.1 | Hs.133130.1 | GenBank | Hs.133130 | | |
| 46 | NM_022917.1 | g12597664 | RefSeq | Hs.183253 | fulllength | 65083 |
| 47 | AF131220.1 | g4589247 | GenBank | Hs.46907 | fulllength | 51409 |
| 48 | BG325646 | Hs.181341.1_RC | GenBank | Hs.347964 | fulllength | 84861 |
| 49 | BF222018 | Hs.289803.0.A1 | GenBank | Hs.2387 | est | 7047 |
| 50 | AI979334 | Hs.323822.0_RC | GenBank | Hs.323822 | | 55019 |

| # | Full_Length_Reference_Seq |
|---|---|
| 1 | NM_018444; pyruvate dehydrogenase phosphatase |
| 2 | NM_018705; hypothetical protein DKFZp547G183 |
| 3 | NM_017745; BCL-6 interacting corepressor isoform 1 |
| | NM_020926; BCL-6 interacting corepressor isoform 2 |
| 4 | NM_003913; serine/threonine-protein kinase PRP4K |
| | NM_176800; serine/threonine-protein kinase PRP4K |
| 5 | |
| 6 | NM_003104; sorbitol dehydrogenase |
| 7 | NM_015902; progestin induced protein |
| 8 | NM_024530; hypothetical protein FLJ23306 |
| 9 | |
| 10 | NM_001659; ADP-ribosylation factor 3 |
| 11 | NM_005146; squamous cell carcinoma antigen recognized by |
| | T cells 1 |
| 12 | NM_001792; cadherin 2, type 1 preproprotein |
| 13 | |
| 14 | NM_006030; calcium channel, voltage-dependent, alpha |
| | 2/delta subunit 2 |
| 15 | NM_024906; hypothetical protein FLJ21032 |
| 16 | NM_003084; small nuclear RNA activating complex, |
| | polypeptide 3, 50 kDa |
| 17 | NM_152465; hypothetical protein MGC39650 |
| 18 | |
| 19 | NM_017763; hypothetical protein FLJ20315 |
| 20 | NM_004350; runt-related transcription factor 3 |
| 21 | NM_014786; Rho-specific guanine-nucleotide exchange |
| | factor 164 kDa |
| 22 | NM_001720; bone morphogenetic protein 8 preproprotein |
| 23 | NM_003852; transcriptional intermediary factor 1 |
| 24 | NM_013328; pyrroline 5-carboxylate reductase isoform |
| 25 | NM_001788; cell division cycle 10 |
| 26 | NM_000318; peroxisomal membrane protein 3 |
| 27 | NM_016463; hypothetical protein HSPC195 |
| 28 | |
| 29 | NM_005595; nuclear factor I/A |
| 30 | |
| 31 | NM_005112; WD repeat-containing protein 1 isoform 2 |
| | NM_017491; WD repeat-containing protein 1 isoform 1 |
| 32 | NM_020168; p21-activated kinase 6 |
| 33 | |
| 34 | NM_024029; hypothetical protein MGC3262 |
| 35 | |
| 36 | |
| 37 | NM_004667; hect domain and RLD 2 |
| 38 | NM_080860; testes specific A2 homolog |
| 39 | NM_002267; karyopherin alpha 3 |
| 40 | NM_005232; EphA1 |
| 41 | NM_004543; nebulin |
| 42 | |
| 43 | NM_014397; putative serine-threonine protein kinase |
| 44 | NM_013296; LGN protein |
| 45 | |
| 46 | NM_022917; nucleolar RNA-associated protein alpha isoform |
| | NM_130793; nucleolar RNA-associated protein beta isoform |
| | NM_139235; nucleolar RNA-associated protein gamma |
| | isoform |
| 47 | NM_016173; HEMK homolog 7 kb |
| 48 | NM_032775; hypothetical protein FLJ14360 |
| 49 | NM_003241; transglutaminase 4 (prostate) |
| 50 | |

TABLE 22
_t(8; 21)

| # | affy Id | HUGO name | Title | MapLocation |
|---|---|---|---|---|
| 1 | 230746_s_at | STC1 | stanniocalcin 1 | 8p21-p11.2 |
| 2 | 239860_at | | ESTs | |
| 3 | 224316_at | FLJ20038 | hypothetical protein FLJ20038 | 8p21.1 |
| 4 | 228615_at | LOC286161 | hypothetical protein LOC286161 | 8p23.3 |
| 5 | 228013_at | | Homo sapiens mRNA; cDNA DKFZp586F1523 (from clone | |
| | | | DKFZp586F1523) | |
| 6 | 202035_s_at | SFRP1 | secreted frizzled-related protein 1 | 8p12-p11.1 |
| 7 | 204865_at | CA3 | carbonic anhydrase III, muscle specific | 8q13-q22 |
| 8 | 230361_at | | ESTs, Weakly similar to inner centromere protein [Mus musculus] | |
| | | | [M. musculus] | |
| 9 | 231268_at | | Homo sapiens , clone IMAGE: 5222754, | |
| | | | mRNA, partial cds | |
| 10 | 223907_s_at | PINX1 | PIN2-interacting protein 1 | 8p23 |
| 11 | 206574_s_at | PTP4A3 | protein tyrosine phosphatase type IVA, | |
| | | | member 3 | |
| 12 | 219060_at | FLJ10204 | hypothetical protein FLJ10204 | 8q24.13 |
| 13 | 202873_at | | Homo sapiens cDNA FLJ33383 fis, clone BRACE2006514. | |
| 14 | 238229_at | | ESTs | |
| 15 | 218273_s_at | PDP | pyruvate dehydrogenase phosphatase | 8q21.3 |
| 16 | 222051_s_at | E2F5 | E2F transcription factor 5, p130-binding | 8q21.13 |
| 17 | 209928_s_at | MSC | musculin (activated B-cell factor-1) | 8q21 |
| 18 | 230661_at | | Homo sapiens mRNA; cDNA DKFZp667P166 (from clone DKFZp667P166) | |
| 19 | 206222_at | TNFRSF10C | tumor necrosis factor receptor superfamily, member 10c, decoy without an intracellular domain | 8p22-p21 |
| 20 | 211489_at | ADRA1A | adrenergic, alpha-1A-, receptor | 8p21-p11.2 |
| 21 | 218777_at | FLJ22246 | hypothetical protein FLJ22246 | 8p21.2 |
| 22 | 212166_at | XPO7 | exportin 7 | 8p21 |
| 23 | 229758_at | TIGD5 | tigger transposable element derived 5 | 8q24.3 |
| 24 | 219231_at | NCOA6IP | nuclear receptor coactivator 6 interacting | 8q11 |
| | | | protein | |
| 25 | 227017_at | LOC157697 | hypothetical protein LOC157697 | 8p23.3 |
| 26 | 225053_at | CNOT7 | CCR4-NOT transcription complex, subunit 7 | 8p22-p21.3 |
| 27 | 218337_at | RAI16 | retinoic acid induced 16 | 8p21.2 |
| 28 | 227263_at | FLJ34715 | hypothetical protein FLJ34715 | 8p21.2 |
| 29 | 209253_at | SCAM-1 | vinexin beta (SH3-containing adaptor | 8p21.2 |
| | | | molecule-1) | |
| 30 | 224413_s_at | BLP1 | BBP-like protein 1 | 8p11.21 |
| 31 | 216865_at | COL14A1 | collagen, type XIV, alpha 1 (undulin) | 8q23 |
| 32 | 205849_s_at | UQCRB | ubiquinol-cytochrome c reductase binding | 8q22 |
| | | | protein | |
| 33 | 222863_at | RINZF | zinc finger protein RINZF | 8q13-q21.1 |
| 34 | 235210_s_at | FLJ40021 | hypothetical protein FLJ40021 | 8q13.2 |
| 35 | 220984_s_at | OATPRP4 | organic anion transporter polypeptide- | 8q13.1 |
| | | | related protein 4 | |
| 36 | 210614_at | TTPA | tocopherol (alpha) transfer protein (ataxia | 8q13.1-q13.3 |
| | | | (Friedreich-like) with vitamin E deficiency) | |
| 37 | 238458_at | LOC286097 | hypothetical protein LOC286097 | 8p22 |
| 38 | 241371_at | | ESTs | |
| 39 | 223819_x_at | HT002 | HT002 protein; hypertension-related calcium-regulated gene | 8q24-qter |
| 40 | 223687_s_at | HSJ001348 | cDNA for differentially expressed CO16 | 8q24.3 |
| | | | gene | |
| 41 | 227836_at | | | |
| 42 | 214708_at | SNTB1 | syntrophin, beta 1 (dystrophin-associated protein A1, 59 kDa, | 8q23-q24 |
| | | | basic component 1) | |
| 43 | 203987_at | FZD6 | frizzled homolog 6 (Drosophila) | 8q22.3-q23.1 |
| 44 | 221020_s_at | MFTC | mitochondrial folate transporter/carrier | 8q22.3 |
| 45 | 238807_at | | Homo sapiens , clone IMAGE: 3957507, | |
| | | | mRNA | |
| 46 | 227001_at | | Homo sapiens cDNA: FLJ21362 fis, clone | |
| | | | COL02886. | |
| 47 | 222665_at | CGI-90 | CGI-90 protein | 8q21.13 |
| 48 | 228225_at | PXMP3 | peroxisomal membrane protein 3, 35 kDa (Zellweger syndrome) | 8q21.1 |
| 49 | 205950_s_at | CA1 | carbonic anhydrase I | 8q13-q22.1 |
| 50 | 220405_at | SNTG1 | syntrophin, gamma 1 | 8q11-q12 |
| 51 | 237802_at | KIAA1889 | KIAA1889 protein | 8q11.22 |
| 52 | 212523_s_at | KIAA0146 | KIAA0146 protein | 8q11.21 |
| 53 | 230411_at | UBE2V2 | ubiquitin-conjugating enzyme E2 variant 2 | 8q11.1 |
| 54 | 213139_at | SNAI2 | snail homolog 2 (Drosophila) | 8q11 |
| 55 | 236556_s_at | FLJ23749 | hypothetical protein FLJ23749 | 8p22 |
| 56 | 216620_s_at | ARHGEF10 | Rho guanine nucleotide exchange factor | 8p23 |
| | | | (GEF) 10 | |
| 57 | 218978_s_at | MSCP | mitochondrial solute carrier protein | 8p21.2 |
| 58 | 204505_s_at | EPB49 | erythrocyte membrane protein band 4.9 | 8p21.1 |
| | | | (dematin) | |
| 59 | 205574_x_at | BMP1 | bone morphogenetic protein 1 | 8p21 |
| 60 | 221805_at | NEFL | neurofilament, light polypeptide 68 kDa | 8p21 |
| 61 | 205667_at | WRN | Werner syndrome | 8p12-p11.2 |
| 62 | 219124_at | FLJ23263 | hypothetical protein FLJ23263 | 8p11.23 |
| 63 | 223976_at | FUT10 | fucosyltransferase 10 (alpha (1,3) | 8p11.23 |
| | | | fucosyltransferase) | |
| 64 | 215404_x_at | FGFR1 | fibroblast growth factor receptor 1 (fms-related tyrosine kinase 2, Pfeiffer | 8p11.2-p11.1 |
| | | | syndrome) | |
| 65 | 221814_at | GPR124 | G protein-coupled receptor 124 | 8p11.1 |
| 66 | 205390_s_at | ANK1 | ankyrin 1, erythrocytic | 8p11.1 |
| 67 | 226547_at | | Homo sapiens mRNA full length insert | |
| | | | cDNA clone EUROIMAGE 1476475 | |
| 68 | 209781_s_at | KHDRBS3 | KH domain containing, RNA binding, signal transduction associated 3 | 8q24.2 |
| 69 | 201644_at | TSTA3 | tissue specific transplantation antigen | 8q24.3 |
| | | | P35B | |

| | | | Sequence | |
|---|---|---|---|---|
| # | Sequence Type | Transcript ID | Derived From | Sequence ID |
| 1 | Consensussequence | Hs.25590.1 | AW003173 | Hs.25590.1.A1 |
| 2 | Consensussequence | Hs.16292.0 | AI311917 | Hs.16292.0.A1 |
| 3 | Exemplarsequence | Hs.72071.1 | AF130091.1 | g11493486 |
| 4 | Consensussequence | Hs.13477.0 | AW291761 | Hs.13477.0_RC |
| 5 | Consensussequence | Hs.61696.0 | AV702575 | Hs.61696.0_RC |
| 6 | Consensussequence | Hs.7306.0 | AI332407 | Hs.7306.0.A1 |
| 7 | Exemplarsequence | Hs.82129.0 | NM_005181.2 | g6996001 |
| 8 | Consensussequence | Hs.146109.0 | AW664013 | Hs.146109.0_RC |
| 9 | Consensussequence | Hs.144027.0 | AI539459 | Hs.144027.0.A1 |
| 10 | Exemplarsequence | Hs.99829.0 | AF205718.1 | g10504237 |
| 11 | Exemplarsequence | Hs.43666.0 | NM_007079.1 | g6857821 |
| 12 | Exemplarsequence | Hs.18029.0 | NM_018024.1 | g8922280 |
| 13 | Consensussequence | Hs.86905.0 | NM_001695.1 | Hs.86905.0 |
| 14 | Consensussequence | Hs.116240.0 | AI187388 | Hs.116240.0.A1 |
| 15 | Exemplarsequence | Hs.22265.0 | NM_018444.1 | g8923959 |
| 16 | Consensussequence | Hs.2331.2 | AW139195 | Hs.2331.2 |
| 17 | Exemplarsequence | Hs.42474.0 | AF060154.1 | g3089604 |
| 18 | Consensussequence | Hs.194024.0 | AW451999 | Hs.194024.0_RC |
| 19 | Exemplarsequence | Hs.119684.0 | NM_003841.1 | g10835042 |
| 20 | Exemplarsequence | Hs.52931.0 | D32201.1 | g927210 |
| 21 | Exemplarsequence | Hs.289063.0 | NM_025232.1 | g13376835 |
| 22 | Consensussequence | Hs.172685.0 | H38643 | Hs.172685.0 |
| 23 | Consensussequence | Hs.71574.0 | AW168771 | Hs.71574.0 |
| 24 | Exemplarsequence | Hs.179909.0 | NM_024831.1 | g13376235 |
| 25 | Consensussequence | Hs.193090.0 | AA767385 | Hs.193090.0.A1 |
| 26 | Consensussequence | Hs.226318.1 | W94952 | Hs.226318.1.A2 |
| 27 | Exemplarsequence | Hs.299148.0 | NM_022749.1 | g12232410 |
| 28 | Consensussequence | Hs.296360.0 | AL519427 | Hs.296360.0.S1 |
| 29 | Exemplarsequence | Hs.33787.1 | AF037261.1 | g3004947 |
| 30 | Exemplarsequence | g13625460 | AF353991.1 | g13625460 |
| 31 | Consensussequence | Hs.36131.2 | M64108.1 | Hs.36131.2.S1 |
| 32 | Exemplarsequence | Hs.131255.0 | NM_006294.1 | g5454151 |
| 33 | Consensussequence | Hs.237146.0 | BG483802 | Hs.237146.0.S2 |
| 34 | Consensussequence | Hs.122544.0 | AW662373 | Hs.122544.0_RC |
| 35 | Exemplarsequence | g13569931 | NM_030958.1 | g13569931 |
| 36 | Exemplarsequence | Hs.69049.0 | U21938.1 | g726181 |
| 37 | Consensussequence | Hs.131044.0 | AI868167 | Hs.131044.0.S1 |
| 38 | Consensussequence | Hs.57851.0 | AW451259 | Hs.57851.0.A1 |
| 39 | Exemplarsequence | Hs.238928.0 | BC003055.1 | g12804384 |
| 40 | Consensussequence | Hs.69517.0 | AA723810 | Hs.69517.0_RC |
| 41 | Consensussequence | Hs.86970.0 | AI859767 | Hs.86970.0.A1 |
| 42 | Consensussequence | Hs.95011.2 | BG484314 | Hs.95011.2 |
| 43 | Exemplarsequence | Hs.114218.0 | NM_003506.1 | g4503830 |
| 44 | Exemplarsequence | g13540550 | NM_030780.1 | g13540550 |
| 45 | Consensussequence | Hs.291531.0 | AW973964 | Hs.291531.0_RC |
| 46 | Consensussequence | Hs.167011.0 | AI096706 | Hs.167011.0 |
| 47 | Consensussequence | Hs.44222.0 | AK000672.1 | Hs.44222.0 |
| 48 | Consensussequence | Hs.180612.2 | AW512586 | Hs.180612.2.A1 |
| 49 | Exemplarsequence | Hs.23118.0 | NM_001738.1 | g4502516 |
| 50 | Exemplarsequence | Hs.167481.0 | NM_018967.1 | g9507162 |
| 51 | Consensussequence | Hs.13390.0 | R54212 | Hs.13390.0.A1 |
| 52 | Consensussequence | Hs.278634.0 | D63480.1 | Hs.278634.0_RC |
| 53 | Consensussequence | Hs.43569.0 | AI800998 | Hs.43569.0_RC |
| 54 | Consensussequence | Hs.93005.0 | AI572079 | Hs.93005.0 |
| 55 | Consensussequence | Hs.174201.0 | AV650622 | Hs.174201.0_RC |
| 56 | Consensussequence | Hs.20695.1 | AF009205.1 | Hs.20695.1 |
| 57 | Exemplarsequence | Hs.283716.0 | NM_018586.1 | g8924059 |
| 58 | Exemplarsequence | Hs.274122.0 | NM_001978.1 | g4503580 |
| 59 | Exemplarsequence | Hs.1274.0 | NM_001199.1 | g4502420 |
| 60 | Consensussequence | Hs.211584.0 | NM_006158.1 | Hs.211584.0_RC |
| 61 | Exemplarsequence | Hs.150477.0 | NM_000553.1 | g5739523 |
| 62 | Exemplarsequence | Hs.288716.0 | NM_025115.1 | g13376690 |
| 63 | Exemplarsequence | Hs.326744.0 | BC004884.1 | g13436133 |
| 64 | Consensussequence | Hs.306678.0 | AK024388.1 | Hs.306678.0.S1 |
| 65 | Consensussequence | Hs.17270.0 | BF511315 | Hs.17270.0 |
| 66 | Exemplarsequence | Hs.183805.0 | NM_000037.2 | g10947039 |
| 67 | Consensussequence | Hs.93231.0 | AI817830 | Hs.93231.0.A1 |
| 68 | Exemplarsequence | Hs.13565.0 | AF069681.1 | g3273831 |
| 69 | Exemplarsequence | Hs.264428.0 | NM_003313.2 | g6598326 |

| | Sequence | | | | |
|---|---|---|---|---|---|
| # | Source | Unigene_Accession | Cluster_Type | LocusLink | Full_Length_Reterence_Seq |
| 1 | GenBank | Hs.25590 | fulllength | 6781 | NM_003155; stanniocalcin 1 |
| 2 | GenBank | Hs.16292 | est | | |
| 3 | GenBank | Hs.72071 | fulllength | 54793 | NM_017634; hypothetical protein FLJ20038 |
| 4 | GenBank | Hs.13477 | | 286161 | |
| 5 | GenBank | Hs.61696 | | | |
| 6 | GenBank | Hs.7306 | fulllength | 6422 | NM_003012; secreted frizzled-related protein 1 |
| 7 | RefSeq | Hs.82129 | fulllength | 761 | NM_005181; carbonic anhydrase III |
| 8 | GenBank | Hs.443139 | est | | |
| 9 | GenBant | Hs.144027 | | | |
| 10 | GenBank | Hs.400259 | fulllength | 54984 | NM_017884; PIN2-interacting protein 1 |
| 11 | RefSeq | Hs.43666 | fulllength | 11156 | NM_007079; protein tyrosine phosphatase type IVA, member 3 isoform 2 |
| | | | | | NM_032611; protein tyrosine phosphatase type IVA, member 3 isoform 1 |
| 12 | RefSeq | Hs.18029 | fulllength | 55093 | NM_018024; hypothetical protein FLJ10204 |
| 13 | GenBank | Hs.290880 | | | |
| 14 | GenBank | Hs.116240 | est | | |
| 15 | RefSeq | Hs.22265 | fulllength | 54704 | NM_018444; pyruvate dehydrogenase phosphatase |
| 16 | GenBank | Hs.2331 | fulllength | 1875 | NM_001951; E2F transcription factor 5 |
| 17 | GenBank | Hs.42474 | fulllength | 9242 | NM_005098; musculin (activated B-cell factor-1) |
| 18 | GenBank | Hs.407120 | | | |
| 19 | RefSeq | Hs.119684 | fulllength | 8794 | NM_003841; tumor necrosis factor receptor superfamily, member 10c |
| | | | | | precursor |
| 20 | GenBank | Hs.52931 | fulllength | 148 | NM_000680; alpha-1A-adrenergic receptor isoform 1 NM_033302; alpha-1A- |
| | | | | | adrenergic receptor isoform 3 NM_033303; alpha-1A-adrenergic receptor |
| | | | | | isoform 2 NM_033304; alpha-1A-adrenergic receptor isoform 4 |
| 21 | RefSeq | Hs.289063 | fulllength | 80346 | NM_025232; hypothetical protein FLJ22246 |
| 22 | GenBank | Hs.172685 | fulllength | 23039 | NM_015024; exportin 7 |
| 23 | GenBank | Hs.71574 | fulllength | 84948 | NM_032862; tigger transposable element derived 5 |
| 24 | RefSeq | Hs.179909 | fulllength | 96764 | NM_024831; PRIP-interacting protein PIPMT |
| 25 | GenBank | Hs.377879 | fulllength | 157697 | |
| 26 | GenBank | Hs.380963 | fulllength | 29883 | NM_013354; CCR4-NOT transcription complex, subunit 7 isoform 1 |
| | | | | | NM_054026; CCR4-NOT transcription complex, subunit 7 isoform 2 |
| 27 | RefSeq | Hs.299148 | fulllength | 64760 | NM_022749; retinoic acid induced 16 |
| 28 | GenBank | Hs.375560 | fulllength | 286057 | NM_173686; hypothetical protein FLJ34715 |
| 29 | GenBank | Hs.33787 | fulllength | 10174 | NM_005775; vinexin beta (SH3-containing adaptor molecule-1) |
| 30 | GenBank | Hs.7471 | fulllength | 83877 | NM_031940; BBP-like protein 1 isoform b NM_078473; BBP-like protein 1 |
| | | | | | isoform a |
| 31 | GenBank | Hs.403836 | fulllength | 7373 | |
| 32 | RefSeq | Hs.131255 | fulllength | 7381 | NM_006294; ubiquinol-cytochrome c reductase binding protein |
| 33 | GenBank | Hs.237146 | fulllength | 65986 | NM_023929; zinc finger protein RINZF |
| 34 | GenBank | Hs.41185 | fulllength | 157869 | NM_153225; hypothetical protein FLJ40021 |
| 35 | RefSeq | Hs.199750 | fulllength | 81796 | NM_030958; organic anion transporter polypeptide-related protein 4 |
| 36 | GenBank | Hs.69049 | fulllength | 7274 | NM_000370; tocopherol (alpha) transfer protein (ataxia (Friedreich-like) with |
| | | | | | vitamin E deficiency) |
| 37 | GenBank | Hs.131044 | fulllength | 286097 | |
| 38 | GenBank | Hs.444578 | est | | |
| 39 | GenBank | Hs.238928 | fulllength | 28991 | NM_014066; hypertension-related calcium-regulated gene |
| 40 | GenBank | Hs.69517 | fulllength | 54742 | NM_017527; cDNA for differentially expressed CO16 gene |
| 41 | GenBank | | | | |
| 42 | GenBank | Hs.95011 | fulllength | 6641 | NM_021021; basic beta 1 syntrophin |
| 43 | RefSeq | Hs.114218 | fulllength | 8323 | NM_003506; frizzled 6 |
| 44 | RefSeq | Hs.196270 | fulllength | 81034 | NM_030780; mitochondrial folate transporter/carrier |
| 45 | GenBank | Hs.291531 | | | |
| 46 | GenBank | Hs.167011 | | | |
| 47 | GenBank | Hs.44222 | fulllength | 51115 | NM_016033; CGI-90 protein |
| 48 | GenBank | Hs.180612 | fulllength | 5828 | NM_000318; peroxisomal membrane protein 3 |
| 49 | RefSeq | Hs.23118 | fulllength | 759 | NM_001738; carbonic anhydrase 1 |
| 50 | RefSeq | Hs.167481 | fulllength | 54212 | NM_018967; syntrophin, gamma 1 |
| 51 | GenBank | Hs.13390 | | 114786 | |
| 52 | GenBank | Hs.278634 | | 23514 | |
| 53 | GenBank | Hs.79300 | fulllength | 7336 | NM_003350; ubiquitin-conjugating enzyme E2 variant 2 |
| 54 | GenBank | Hs.93005 | fulllength | 6591 | NM_003068; snail 2 |
| 55 | GenBank | Hs.180178 | fulllength | 91694 | NM_152271; hypothetical protein FLJ23749 |
| 56 | GenBank | Hs.20695 | fulllength | 9639 | NM_014629; Rho guanine nucleotide exchange factor 10 |
| 57 | RefSeq | Hs.283716 | fulllength | 51312 | NM_016612; mitochondrial solute carrier protein NM_018579; mitochondrial |
| | | | | | solute carrier protein |
| 58 | RefSeq | Hs.274122 | fulllength | 2039 | NM_001978; erythrocyte membrane protein band 4.9 (dematin) |
| 59 | RefSeq | Hs.1274 | fulllength | 649 | NM_001199; bone morphogenetic protein 1 isoform 1, precursor |
| | | | | | NM_006128; bone morphogenetic protein 1 isoform 2, precursor |
| | | | | | NM_006129; bone morphogenetic protein 1 isoform 3, precursor |
| | | | | | NM_006130; bone morphogenetic protein 1 isoform 6 precursor |
| | | | | | NM_006131; bone morphogenetic protein 1 isoform 5, precursor |
| | | | | | NM_006132; bone morphogenetic protein 1 isoform 4, precursor |
| 60 | GenBank | Hs.211584 | fulllength | 4747 | NM_006158; neurofilament, light polypeptide 68 kDa |
| 61 | RefSeq | Hs.150477 | fulllength | 7486 | NM_000553; Werner syndrome protein |
| 62 | RefSeq | Hs.288716 | fulllength | 80185 | NM_025115; hypothetical protein FLJ23263 |
| 63 | GenBank | Hs.326744 | fulllength | 84750 | NM_032664; fucosyltransferase 10 |
| 64 | GenBank | Hs.748 | fulllength | 2260 | NM_000604; fibroblast growth factor receptor 1 isoform 1 precursor |
| | | | | | NM_015850; fibroblast growth factor receptor 1 isoform 2 precursor |
| | | | | | NM_023105; fibroblast growth factor receptor 1 isoform 3 precursor |
| | | | | | NM_023106; fibroblast growth factor receptor 1 isoform 4 precursor |
| | | | | | NM_023107; fibroblast growth factor receptor 1 isoform 5 precursor |
| | | | | | NM_023108; fibroblast growth factor receptor 1 isoform 6 precursor |
| | | | | | NM_023109; fibroblast growth factor receptor 1 isoform 7 precursor |
| | | | | | NM_023110; fibroblast growth factor receptor 1 isoform 8 precursor |
| | | | | | NM_023111; fibroblast growth factor receptor 1 isoform 9 precursor |
| 65 | GenBank | Hs.17270 | fulllength | 25960 | NM_032777; G protein-coupled receptor 124 |
| 66 | RefSeq | Hs.183805 | fulllength | 286 | NM_000037; ankyrin 1 isoform 3 NM_020475; ankyrin 1 isoform 4 |
| | | | | | NM_020476; ankyrin 1 isoform 1 NM_020477; ankyrin 1 isoform 2 |
| | | | | | NM_020478; ankyrin 1 isoform 5 NM_020479; ankyrin 1 isoform 6 |
| | | | | | NM_020480; ankyrin 1 isoform 7 NM_020481; ankyrin 1 isoform 8 |
| 67 | GenBank | Hs.93231 | | | |
| 68 | GenBank | Hs.13565 | fulllength | 10656 | NM_006558; KH domain containing, RNA binding, signal transduction |
| | | | | | associated 3 |
| 69 | RefSeq | Hs.404119 | fulllength | 7264 | NM_003313; tissue specific transplantation antigen P35B |

TABLE 23
_t(15; 17)

| # | affy id | HUGO name | F | p | q | Title | MapLocation | Sequence Type | Transcript ID | Sequence Derived From |
|---|---|---|---|---|---|---|---|---|---|---|
| 1 | 225032_at | FAD104 | 66.70 | 3.94e−10 | 1.40e−05 | FAD104 | 3q26.31 | Consensussequence | Hs.299883.1 | AI141784 |
| 2 | 204274_at | EBAG9 | 55.79 | 7.06e−08 | 4.18e−04 | estrogen receptor binding site | 8q23 | Consensussequence | Hs.9222.0 | AA812215 |
| | | | | | | associated, antigen, 9 | | | | |
| 3 | 230541_at | LOC149134 | 48.92 | 3.43e−08 | 3.05e−04 | hypothetical protein LOC149134 | 1q44 | Consensussequence | Hs.61829.0 | AU150080 |
| 4 | 210115_at | RPL39L | 47.31 | 2.59e−08 | 3.05e−04 | ribosomal protein L39-like | 3q27 | Exemplarsequence | Hs.132748.0 | L05096.1 |
| 5 | 235118_at | | 47.06 | 2.60e−08 | 3.05e−04 | Homo sapiens cDNA FLJ38226 fis, | | Consensussequence | Hs.12382.0 | AV724769 |
| | | | | | | clone FCBBF2004066. | | | | |
| 6 | 222365_at | | 45.14 | 4.90e−08 | 3.48e−04 | ESTs | | Consensussequence | Hs.293024.0 | AW974666 |
| 7 | 222336_at | | 41.89 | 9.35e−08 | 4.75e−04 | ESTs | | Consensussequence | Hs.116550.0 | AW974915 |
| 8 | 207995_s_at | CD209L | 41.60 | 1.08e−07 | 4.79e−04 | CD209 antigen-like | 19p13 | Exemplarsequence | Hs.23759.0 | NM_014257.1 |
| 9 | 217478_s_at | HLA-DMA | 41.33 | 1.44e−07 | 5.68e−04 | major histocompatibility complex, class II, DM | 6p21.3 | Consensussequence | Hs.77522.1 | X76775 |
| | | | | | | alpha | | | | |
| 10 | 230747_s_at | | 37.74 | 2.76e−07 | 9.80e−04 | Homo sapiens cDNA FLJ90394 fis, | | Consensussequence | Hs.112011.0 | AA406435 |
| | | | | | | clone NT2RP2005632. | | | | |
| 11 | 204424_s_at | DAT1 | 37.64 | 3.07e−05 | 1.54e−02 | neuronal specific transcription factor DAT1 | 12p12.3 | Consensussequence | Hs.301914.0 | AL050152.1 |
| 12 | 235748_s_at | | 37.46 | 3.54e−07 | 1.14e−03 | Homo sapiens, clone IMAGE: 4830703, mRNA, | | Consensussequence | Hs.105094.0 | AW969382 |
| | | | | | | partial cds | | | | |
| 13 | 218017_s_at | FLJ22242 | 36.67 | 5.88e−05 | 1.97e−02 | hypothetical protein FLJ22242 | 8p11.1 | Exemplarsequence | Hs.288057.0 | NM_025070.1 |
| 14 | 216630_at | | 36.34 | 8.81e−07 | 1.96e−03 | Homo sapiens mRNA; cDNA | | Consensussequence | Hs.306338.0 | AL110190.1 |
| | | | | | | DKFZp564J2116 (from clone | | | | |
| | | | | | | DKFZp564J2116) | | | | |
| 15 | 216781_at | KIAA1751 | 36.19 | 4.59e−07 | 1.36e−03 | KIAA1751 protein | 1p36.33 | Consensussequence | Hs.307002.0 | AB051538.1 |
| 16 | 211838_x_at | PCDHA5 | 34.85 | 5.97e−07 | 1.62e−03 | protocadherin alpha 5 | 5q31 | Exemplarsequence | Hs.167399.1 | AF152483.1 |
| 17 | 227860_at | CPXM | 34.64 | 6.39e−07 | 1.62e−03 | carboxypeptidase X (M14 family) | 20p12.3-p13 | Consensussequence | Hs.177536.0 | NM_019609.1 |
| 18 | 222350_at | | 34.43 | 1.14e−06 | 2.38e−03 | ESTs | | Consensussequence | Hs.105121.0 | AW969913 |
| 19 | 201240_s_at | KIAA0102 | 34.21 | 7.32e−07 | 1.73e−03 | KIAA0102 gene product | 11q13.3 | Exemplarsequence | Hs.77665.0 | NM_014752.1 |
| 20 | 230157_at | CDH24 | 33.71 | 1.95e−06 | 3.58e−03 | cadherin-like 24 | 14q11.2 | Consensussequence | Hs.155912.0 | AL137477.1 |
| 21 | 235429_at | EIF3S6 | 33.32 | 6.51e−06 | 6.18e−03 | eukaryotic translation initiation factor 3, | 8q22-q23 | Consensussequence | Hs.161623.0 | AW965494 |
| | | | | | | subunit 6 48 kDa | | | | |
| 22 | 205674_x_at | FXYD2 | 31.85 | 4.02e−06 | 5.49e−03 | FXYD domain containing ion transport | 11q23 | Exemplarsequence | Hs.19520.1 | NM_001680.2 |
| | | | | | | regulator 2 | | | | |
| 23 | 237484_at | | 31.72 | 2.12e−06 | 3.58e−03 | Homo sapiens cDNA FLJ35318 fis, | | Consensussequence | Hs.220931.0 | BE501385 |
| | | | | | | clone PROST2011410. | | | | |
| 24 | 223800_s_at | LOC96626 | 31.50 | 1.16e−05 | 8.27e−03 | pinch-2 | 2q14-q21 | Exemplarsequence | Hs.285130.0 | AF288404.1 |
| 25 | 230765_at | KIAA1239 | 30.59 | 2.09e−06 | 3.58e−03 | KIAA1239 protein | 4p14 | Consensussequence | Hs.4280.0 | AL037517 |
| 26 | 210882_s_at | TRO | 30.44 | 2.12e−06 | 3.58e−03 | trophinin | Xp11.22-p11.21 | Exemplarsequence | Hs.259802.1 | U04811.1 |
| 27 | 209810_at | SFTPB | 30.30 | 2.23e−06 | 3.60e−03 | surfactant, pulmonary-associated | 2p12-p11.2 | Exemplarsequence | Hs.76305.0 | J02761.1 |
| | | | | | | protein B | | | | |
| 28 | 205325_at | PHYHIP | 29.94 | 2.78e−06 | 4.30e−03 | phytanoyl-CoA hydroxylase interacting | 8p21.2 | Exemplarsequence | Hs.239500.0 | NM_014759.1 |
| | | | | | | protein | | | | |
| 29 | 231226_at | CRSP2 | 29.76 | 7.40e−06 | 6.75e−03 | cofactor required for Sp1 | Xp11.4-p11.2 | Consensussequence | Hs.135174.0 | BF196752 |
| | | | | | | transcriptional activation, subunit 2, | | | | |
| | | | | | | 150 kDa | | | | |
| 30 | 216278_at | | 29.34 | 2.92e−06 | 4.33e−03 | Homo sapiens mRNA full length insert | | Consensussequence | Hs.9997.0 | AL109705.1 |
| | | | | | | cDNA clone EUROIMAGE 73337. | | | | |
| 31 | 204153_s_at | MFNG | 38259 | 1.96e−05 | 1.21e−02 | manic fringe homolog (Drosophila) | 22q12 | Exemplarsequence | Hs.31939.0 | NM_002405.1 |
| 32 | 206229_x_at | PAX2 | 28.80 | 3.43e−06 | 4.87e−03 | paired box gene 2 | 10q22.1-q24.3 | Exemplarsequence | Hs.155644.0 | NM_003988.1 |
| 33 | 222692_s_at | FAD104 | 28.73 | 5.34e−04 | 5.03e−02 | FAD104 | 3q26.31 | Consensussequence | Hs.299883.0 | BF444916 |
| 34 | 242417_at | LOC283278 | 28.52 | 1.94e−05 | 1.21e−02 | hypothetical protein LOC283278 | 11p15.1 | Consensussequence | Hs.201661.0 | AI690465 |
| 35 | 227757_at | CUL4A | 28.47 | 5.89e−06 | 6.09e−03 | cullin 4A | 13q34 | Consensussequence | Hs.183874.1 | AL563297 |
| 36 | 224629_at | | 38258 | 5.50e−06 | 6.09e−03 | Homo sapiens cDNA: FLJ22120 fis, | | Consensussequence | Hs.5822.0 | BF217539 |
| | | | | | | clone HEP18874. | | | | |
| 37 | 205548_s_at | BTG3 | 38196 | 8.24e−06 | 7.32e−03 | BTG family, member 3 | 21q21.1-q21.2 | Exemplarsequence | Hs.77311.0 | NM_006806.1 |
| 38 | 49679_s_at | MMP24 | 28.00 | 4.37e−06 | 5.74e−03 | matrix metalloproteinase 24 | 20q11.2 | Consensussequence | 5 | AA243774 |
| | | | | | | (membrane-inserted) | | | | |
| 39 | 241645_at | | 27.99 | 4.52e−06 | 5.74e−03 | | | Consensussequence | Hs.293467.0 | AW974844 |
| 40 | 237838_at | | 27.79 | 5.99e−06 | 6.09e−03 | Homo sapiens, clone IMAGE: 5271699, | | Consensussequence | Hs.134004.0 | AI075924 |
| | | | | | | mRNA | | | | |
| 41 | 234702_x_at | CFTR | 27.70 | 5.17e−06 | 6.09e−03 | cystic fibrosis transmembrane | 7q31.2 | Consensussequence | Hs.326797.0 | S64699.1 |
| | | | | | | conductance regulator, ATP-binding | | | | |
| | | | | | | cassette (sub-family C, member 7) | | | | |
| 42 | 208011_at | PTPN22 | 27.54 | 5.18e−06 | 6.09e−03 | protein tyrosine phosphatase, non- | 1p13.3-p13.1 | Exemplarsequence | Hs.87860.1 | NM_012411.1 |
| | | | | | | receptor type 22 (lymphoid) | | | | |
| 43 | 226165_at | E2F5 | 27.51 | 5.15e−05 | 1.90e−02 | E2F transcription factor 5, p130- | 8q21.13 | Consensussequence | Hs.286124.2 | BF674436 |
| | | | | | | binding | | | | |
| 44 | 222579_at | FLJ23251 | 27.34 | 5.67e−06 | 6.09e−03 | hypothetical protein FLJ23251 | 3q22.1 | Consensussequence | Hs.170737.0 | NM_024818.1 |
| 45 | 222632_s_at | LZTFL1 | 27.33 | 6.25e−05 | 2.08e−02 | leucine zipper transcription factor-like 1 | 3p21.3 | Consensussequence | Hs.30824.0 | AA843132 |
| 46 | 217170_at | TRA | 27.79 | 5.89e−06 | 6.09e−03 | T cell receptor alpha locus | 14q11.2 | Consensussequence | Hs.247915.0 | AE000659 |
| 47 | 226585_at | NEIL2 | 26.89 | 9.83e−04 | 6.70e−02 | nei like 2 (E. coli) | | Consensussequence | Hs.293818.0 | BE466620 |
| 48 | 203139_at | DAPK1 | 26.80 | 6.34e−06 | 6.18e−03 | death-associated protein kinase 1 | 9q34.1 | Exemplarsequence | Hs.153924.0 | NM_004938.1 |
| 49 | 225018_at | Spir-1 | 26.70 | 6.61e−06 | 6.18e−03 | Spir-1 protein | 18p11.1 | Consensussequence | Hs.16758.0 | AJ277587.1 |
| 50 | 214538_x_at | RGS6 | 26.46 | 1.05e−04 | 2.54e−02 | regulator of G-protein signalling 6 | 14q24.3 | Consensussequence | Hs.3221.2 | AF073921.1 |

| | Sequence | Sequence | | | | |
|---|---|---|---|---|---|---|
| # | ID | Source | Unigene_Accession | Cluster_Type | LocusLink | Full_Length_Reference_Seq |
| 1 | Hs.299883.1.S1 | GenBank | Hs.299883 | fulllength | 64778 | NM_022763; FAD104 |
| 2 | Hs.9222.0 | GenBank | Hs.9222 | fulllength | 9166 | NM_004215; estrogen receptor binding site associated antigen 9 |
| 3 | Hs.61829.0 | GenBank | Hs.417101 | | 149134 | |
| 4 | g388035 | GenBank | Hs.132748 | fulllength | 116832 | NM_052969; ribosomal protein L39-like protein |
| 5 | Hs.12382.0 | GenBank | Hs.12382 | | | |
| 6 | Hs.293024.0.A1 | GenBank | Hs.293024 | est | | |
| 7 | Hs.116550.0_RC | GenBank | Hs.430765 | est | | |
| 8 | g7657173 | RefSeq | Hs.23759 | fulllength | 10332 | NM_014257; CD209 antigen-like |
| 9 | Hs.77522.1 | GenBank | Hs.77522 | fulllength | 3108 | NM_006120; major histocompatibility complex, class II, DM alpha precursor |
| 10 | Hs.112011.0.S1 | GenBank | Hs.112011 | | | |
| 11 | Hs.301914.0_RC | GenBank | Hs.301914 | fulllength | 55885 | NM_018640; neuronal specific transcription factor DAT1 |
| 12 | Hs.105094.0.S1 | GenBank | Hs.407156 | | | |
| 13 | g13376612 | RefSeq | Hs.288057 | fulllength | 80140 | NM_025070; hypothetical protein FLJ22242 |
| 14 | Hs.306338.0.A1 | GenBank | Hs.306338 | | | |
| 15 | Hs.307002.0.A1 | GenBank | Hs.307002 | | 85452 | |
| 16 | g5457014 | GenBank | Hs.167399 | fulllength | 56143 | NM_018908; protocadherin alpha 5 isoform 1 precursor NM_031501; |
| | | | | | | protocadherin alpha 5 isoform 2 precursor |
| 17 | Hs.177536.0 | GenBank | Hs.177536 | fulllength | 56265 | NM_019609; metallocarboxypeptidase CPX-1 precursor |
| 18 | Hs.105121.0 | GenBank | Hs.432532 | est | | |
| 19 | g7661907 | RefSeq | Hs.77665 | fulllength | 9789 | NM_014752; KIAA0102 gene product |
| 20 | Hs.155912.0 | GenBank | Hs.155912 | fulllength | 64403 | NM_022478; cadherin-like 24 |
| 21 | Hs.161623.0.A1 | GenBank | Hs.106673 | fulllength | 3646 | NM_001568; murine mammary tumor integration site 6 (oncogene homolog) |
| 22 | g11125765 | RefSeq | Hs.19520 | fulllength | 486 | NM_001680; FXYD domain-containing ion transport regulator 2 isoform 1 |
| | | | | | | NM_021603; FXYD domain-containing ion transport regulator 2 isoform 2 |
| 23 | Hs.220931.0_RC | GenBank | Hs.220931 | | | |
| 24 | g9800508 | GenBank | Hs.285130 | fulllength | 96626 | NM_033514; pinch-2 |
| 25 | Hs.4280.0.S1 | GenBank | Hs.4280 | | 57495 | |
| 26 | g905357 | GenBank | Hs.259802 | fulllength | 7216 | NM_016157; trophinin isoform 2 NM_177555; trophinin isoform 1 NM_177556; |
| | | | | | | trophinin isoform 2 NM_177557; trophinin isoform 4 NM_177558; trophinin |
| | | | | | | isoform 3 |
| 27 | g190673 | GenBank | Hs.76305 | fulllength | 6439 | NM_000542; surfactant, pulmonary-associated protein B |
| 28 | g7662031 | RefSeq | Hs.334688 | fulllength | 9796 | NM_014759; phytanoyl-CoA hydroxylase interacting protein |
| 29 | Hs.135174.0.A1 | GenBank | Hs.407604 | fulllength | 9282 | NM_004229; cofactor required for Sp1 transcriptional activation, subunit 2, |
| | | | | | | 150 kDa |
| 30 | Hs.9997.0 | GenBank | Hs.9997 | | | |
| 31 | g4505158 | RefSeq | Hs.31939 | fulllength | 4242 | NM_002405; manic fringe (Drosophila) homolog |
| 32 | g4557824 | RefSeq | Hs.155644 | fulllength | 5076 | NM_000278; paired box protein 2 isoform b NM_003987; paired box protein 2 |
| | | | | | | isoform a NM_003988; paired box protein 2 isoform c NM_003989; paired box |
| | | | | | | protein 2 isoform d NM_003990; paired box protein 2 isoform e |
| 33 | Hs.299883.0.S2 | GenBank | Hs.299883 | fulllength | 64778 | NM_022763; FAD104 |
| 34 | Hs.201661.0.A1 | GenBank | Hs.201661 | | 283278 | |
| 35 | Hs.183874.1.S1 | GenBank | Hs.183874 | fulllength | 8451 | NM_003589; cullin 4A |
| 36 | Hs.5822.0.S1 | GenBank | Hs.5822 | | | |
| 37 | g5802989 | RefSeq | Hs.77311 | fulllength | 10950 | NM_006806; B-cell translocation gene 3 |
| 38 | 4921436_rc | GenBank | Hs.3743 | fulllength | 10893 | NM_006690; matrix metalloproteinase 24 (membrane-inserted) |
| 39 | Hs.293467.0_RC | GenBank | | | | |
| 40 | Hs.134004.0.A1 | GenBank | Hs.134004 | | | |
| 41 | Hs.326797.0 | GenBank | Hs.426266 | fulllength | 1080 | NM_000492; cystic fibrosis transmembrane conductance regulator, ATP-binding |
| | | | | | | cassette (sub-family C, member 7) |
| 42 | g6912613 | RefSeq | Hs.87860 | fulllength | 26191 | NM_012411; lymphoid-specific protein tyrosine phosphatase isoform 2 |
| | | | | | | NM_015967; lymphoid-specific protein tyrosine phosphatase isoform 1 |
| 43 | Hs.286124.2_RC | GenBank | Hs.2331 | fulllength | 1875 | NM_001951; E2F transcription factor 5 |
| 44 | Hs.170737.0_RC | GenBank | Hs.170737 | fulllength | 79876 | NM_024818; hypothetical protein FLJ23251 |
| 45 | Hs.30824.0.S1 | GenBank | Hs.30824 | fulllength | 54585 | NM_020347; leucine zipper transcription factor-like 1 |
| 46 | Hs.247915.0 | GenBank | Hs.74647 | fulllength | 6955 | |
| 47 | Hs.293818.0.A1 | GenBank | Hs.293818 | fulllength | 252969 | NM_145043; nei-like 2 |
| 48 | g4826683 | RefSeq | Hs.153924 | fulllength | 1612 | NM_004938; death-associated protein kinase 1 |
| 49 | Hs.16758.0 | GenBank | Hs.16758 | fulllength | 56907 | |
| 50 | Hs.3221.2.S1 | GenBank | Hs.3221 | fulllength | 9628 | NM_004296; regulator of G-protein signalling 6 |

TABLE 24
_t(15; 17)

| # | affy Id | HUGO name | Title | MapLocation | Sequence Type | Transcript ID | Sequence Derived From | Sequence ID |
|---|---|---|---|---|---|---|---|---|
| 1 | 230746_s_at | STC1 | stanniocalcin 1 | 8p21-p11.2 | Consensussequence | Hs.25590.1 | AW003173 | Hs.25590.1.A1 |
| 2 | 239860_at | | ESTs | | Consensussequence | Hs.16292.0 | AI311917 | Hs.16292.0.A1 |
| 3 | 209928_s_at | MSC | musculin (activated B-cell factor-1) | 8q21 | Exemplarsequence | Hs.42474.0 | AF060154.1 | g3089604 |
| 4 | 206222_at | TNFRSF10C | tumor necrosis factor receptor superfamily, | 8p22-p21 | Exemplarsequence | Hs.119684.0 | NM_003841.1 | g10835042 |
| | | | member 10c, decoy without an intracellular domain | | | | | |
| 5 | 211489_at | ADRA1A | adrenergic, alpha-1A-, receptor | 8p21-p11.2 | Exemplarsequence | Hs.52931.0 | D32201.1 | g927210 |
| 6 | 218777_at | FLJ22246 | hypothetical protein FLJ22246 | 8p21.2 | Exemplarsequence | Hs.289063.0 | NM_025232.1 | g13376835 |
| 7 | 212166_at | XPO7 | exportin 7 | 8p21 | Consensussequence | Hs.172685.0 | H38643 | Hs.172685.0 |
| 8 | 225053_at | CNOT7 | CCR4-NOT transcription complex, subunit 7 | 8p22-p21.3 | Consensussequence | Hs.226318.1 | W94952 | Hs.226318.1.A2 |
| 9 | 227263_at | FLJ34715 | hypothetical protein FLJ34715 | 8p21.2 | Consensussequence | Hs.296360.0 | AL519427 | Hs.296360.0.S1 |
| 10 | 224413_s_at | BLP1 | BBP-like protein 1 | 8p11.21 | Exemplarsequence | g13625460 | AF353991.1 | g13625460 |
| 11 | 205849_s_at | UQCRB | ubiquinol-cytochrome c reductase binding | 8q22 | Exemplarsequence protein | Hs.131255.0 | NM_006294.1 | g5454151 |
| 12 | 235210_s_at | FLJ40021 | hypothetical protein FLJ40021 | 8q13.2 | Consensussequence | Hs.122544.0 | AW662373 | Hs.122544.0_RC |
| 13 | 227001_at | | Homo sapiens cDNA: FLJ21362 fis clone | | Consensussequence | Hs.167011.0 | AI096706 | Hs.167011.0 |
| | | | COL02886. | | | | | |
| 14 | 222665_at | CGI-90 | CGI-90 protein | 8q21.13 | Consensussequence | Hs.44222.0 | AK000672.1 | Hs.44222.0 |
| 15 | 212523_s_at | KIAA0146 | KIAA0146 protein | 8q11.21 | Consensussequence | Hs.278634.0 | D63480.1 | Hs.278634.0_RC |
| 16 | 230411_at | UBE2V2 | ubiquitin-conjugating enzyme E2 variant 2 | 8q11.1 | Consensussequence | Hs.43569.0 | AI800998 | Hs.43569.0_RC |
| 17 | 219124_at | FLJ23263 | hypothetical protein FLJ23263 | 8p11.23 | Exemplarsequence | Hs.288716.0 | NM_025115.1 | g13376690 |
| 18 | 201644_at | TSTA3 | tissue specific transplantation antigen P35B | 8q24.3 | Exemplarsequence | Hs.264428.0 | NM_003313.2 | g6598326 |
| 19 | 201398_s_at | TRAM | translocating chain-associating membrane | 8q13.1 | Exemplarsequence | Hs.4147.0 | BC000687.1 | g12653796 |
| | | | protein | | | | | |
| 20 | 235509_at | MGC40214 | hypothetical protein MGC40214 | 8q22.1 | Consensussequence | Hs.98471.0 | AV662196 | Hs.98471.0_RC |
| 21 | 203208_s_at | CHPPR | likely ortholog of chicken chondrocyte | 8q12.1 | Exemplarsequence | Hs.170198.0 | NM_014637.1 | g7661853 |
| | | | protein with a poly-proline region | | | | | |
| 22 | 218899_s_at | BAALC | brain and acute leukemia, cytoplasmic | 8q22.3 | Exemplarsequence | Hs.169395.0 | NM_024812.1 | g13376199 |
| 23 | 230016_at | | Homo sapiens cDNA FLJ13277 fis, clone | | Consensussequence | Hs.55043.0 | AU155118 | Hs.55043.0.S1 |
| | | | OVARC1001044. | | | | | |
| 24 | 232693_s_at | LOC55893 | papillomavirus regulatory factor PRF-1 | 8p21.1 | Consensussequence | Hs.27410.2 | AK021850.1 | Hs.27410.2.S1 |
| 25 | 203207_s_at | CHPPR | likely ortholog of chicken chondrocyte | 8q12.1 | Consensussequence | Hs.170198.0 | BF214329 | Hs.170198.0.S1 |
| | | | protein with a poly-proline region | | | | | |
| 26 | 212248_at | | Homo sapiens, Similar to LYRIC, clone | | Consensussequence | Hs.243901.0 | AI972475 | Hs.243901.0.S1 |
| | | | MGC: 41931 IMAGE: 5298467, mRNA, | | | | | |
| | | | complete cds | | | | | |
| 27 | 202680_at | GTF2E2 | general transcription factor IIE, polypeptide | 8p21-p12 | Exemplarsequence | Hs.77100.0 | NM_002095.1 | g4504194 |
| | | | 2, beta 34 kDa | | | | | |
| 28 | 208791_at | CLU | clusterin (complement lysis inhibitor, SP- | 8p21-p12 | Exemplarsequence | Hs.75106.0 | M25915.1 | g180619 |
| | | | 40,40, sulfated glycoprotein 2, testosterone- | | | | | |
| | | | repressed prostate message 2, | | | | | |
| | | | apolipoprotein J) | | | | | |
| 29 | 208792_s_at | CLU | clusterin (complement lysis inhibitor, SP- | 8p21-p12 | Exemplarsequence | Hs.75106.0 | M25915.1 | g180619 |
| | | | 40,40, sulfated glycoprotein 2, testosterone- | | | | | |
| | | | repressed prostate message 2, | | | | | |
| | | | apolipoprotein J) | | | | | |
| 30 | 221236_s_at | STMN4 | stathmin-like 4 | 8p21.1 | Exemplarsequence | g13540510 | NM_030795.1 | g13540510 |
| 31 | 211535_s_at | FGFR1 | fibroblast growth factor receptor 1 (fms- | 8p11.2-p11.1 | Exemplarsequence | Hs.748.6 | M60485.1 | g182560 |
| | | | related tyrosine kinase 2, Pfeiffer syndrome) | | | | | |
| 32 | 218173_s_at | WHSC1L1 | Wolf-Hirschhom syndrome candidate 1-like | 8p11.2 | Exemplarsequence | Hs.27721.0 | NM_017778.2 | g13699812 |
| 33 | 225676_s_at | DKFZP564O0463 | DKFZP564O0463 protein | 8q22.3 | Consensussequence | Hs.273344.1 | AK001693.1 | Hs.273344.1 |
| 34 | 218187_s_at | FLJ20989 | hypothetical protein FLJ20989 | | Exemplarsequence | Hs.169615.0 | NM_023080.1 | g12751496 |
| 35 | 226483_at | FLJ32370 | hypothetical protein FLJ32370 | 8q11.23 | Consensussequence | Hs.280858.1 | AI890761 | Hs.280858.1.A1 |
| 36 | 208647_at | FDFT1 | farnesyl-diphosphate farnesyltransferase 1 | 8p23.1-p22 | Consensussequence | Hs.48876.1 | AA872727 | Hs.48876.1 |
| 37 | 200090_at- | FNTA | farnesyltransferase, CAAX box, alpha | 8p22-q11 | Consensussequence | Hs.138381.1 | BG168896 | Hs.138381.1.A1 |
| | HG-U133A | | | | | | | |
| 38 | 218250_s_at | CNOT7 | CCR4-NOT transcription complex, subunit 7 | 8p22-p21.3 | Exemplarsequence | Hs.226318.0 | NM_013354.2 | g10518495 |
| 39 | 202174_s_at | PCM1 | pericentriolar material 1 | 8p22-p21.3 | Exemplarsequence | Hs.75737.0 | NM_006197.1 | g5453855 |
| 40 | 214118_x_at | PCM1 | pericentriolar material 1 | 8p22-p21.3 | Consensussequence | Hs.75737.1 | AI205598 | Hs.75737.1.S1 |
| 41 | 214937_x_at | PCM1 | pericentriolar material 1 | 8p22-p21.3 | Consensussequence | Hs.75737.2 | AI924817 | Hs.75737.2.S1 |
| 42 | 222544_s_at | WHSC1L1 | Wolf-Hirschhorn syndrome candidate 1-like 1 | 8p11.2 | Consensussequence | Hs.27721.0 | AI697751 | Hs.27721.0_RC |
| 43 | 219819_s_at | MRPS28 | mitochondrial ribosomal protein S28 | 8q21.1-q21.2 | Exemplarsequence | Hs.55097.0 | NM_014018.1 | g7661729 |
| 44 | 210029_at | INDO | indoleamine-pyrrole 2,3 dioxygenase | 8p12-p11 | Exemplarsequence | Hs.840.0 | M34455.1 | g185790 |
| 45 | 201618_x_at | GPAA1 | GPAA1P anchor attachment protein 1 | 8q24.3 | Exemplarsequence | Hs.4742.0 | NM_003801.2 | g6031166 |
| | | | homolog (yeast) | | | | | |
| 46 | 200936_at | RPL8 | ribosomal protein L8 | 8q24.3 | Exemplarsequence | Hs.178551.0 | NM_000973.1 | g4506662 |
| 47 | 213072_at | LOC157542 | hypothetical protein BC004544 | 8q24.3 | Consensussequence | Hs.331601.0 | AI928387 | Hs.331601.0 |
| 48 | 58696_at | FLJ20591 | exosome component Rrp41 | 8q24.3 | Consensussequence | 5 | AL039469 | 4923514_rc |
| 49 | 236533_at | DDEF1 | development and differentiation enhancing | 8q24.1-q24.2 | Consensussequence | Hs.199057.0 | AW236958 | Hs.199057.0.A1 |
| | | | factor 1 | | | | | |
| 50 | 238562_at | | ESTs, Weakly similar to hypothetical protein | | Consensussequence | Hs.280297.0 | BE542779 | Hs.280297.0.A1 |
| | | | FLJ20378 [Homo sapiens] [H. sapiens] | | | | | |
| 51 | 219548_at | ZNF16 | zinc finger protein 16 (KOX 9) | 8q24 | Exemplarsequence | Hs.23019.0 | NM_006958.1 | g11177859 |
| 52 | 212250_at | | Homo sapiens, Similar to LYRIC, clone | | Consensussequence | Hs.243901.0 | AI972475 | Hs.243901.0.S1 |
| | | | MGC: 41931 IMAGE: 5298467, mRNA, | | | | | |
| | | | complete cds | | | | | |
| 53 | 227277_at | | Homo sapiens cDNA FLJ40968 fis, clone | | Consensussequence | Hs.33074.0 | BG530089 | Hs.33074.0.S1 |
| | | | UTERU2012615. | | | | | |
| 54 | 235391_at | LOC137392 | similar to CG6405 gene product | 8q21.3 | Consensussequence | Hs.87672.0 | AW960748 | Hs.87672.0_RC |
| 55 | 202119_s_at | CPNE3 | copine III | 8q21.13 | Exemplarsequence | Hs.14158.0 | NM_003909.1 | g4503014 |
| 56 | 218919_at | FLJ14007 | hypothetical protein FLJ14007 | 8q21.12 | Exemplarsequence | Hs.99519.0 | NM_024699.1 | g13375984 |
| 57 | 208361_s_at | BN51T | BN51 (BHK21) temperature sensitivity | 8q21 | Exemplarsequence | Hs.1276.0 | NM_001722.1 | g4502436 |
| | | | complementing | | | | | |
| 58 | 223475_at | LOC83690 | CocoaCrisp | 8q13.3 | Exemplarsequence | Hs.182364.0 | AF142573.1 | g12002310 |
| 59 | 202955_s_at | BIG1 | brefeldin A-inhibited guanine nucleotide- | 8q13 | Exemplarsequence | Hs.94631.0 | AF084520.1 | g5052120 |
| | | | exchange protein 1 | | | | | |
| 60 | 203448_s_at | TERF1 | telomeric repeat binding factor (NIMA- | 8q13 | Consensussequence | Hs.194562.0 | AI347136 | Hs.194562.0.A1 |
| | | | interacting) 1 | | | | | |
| 61 | 204301_at | KIAA0711 | KIAA0711 gene product | 8p23.2 | Exemplarsequence | Hs.5333.0 | NM_014867.1 | g7662259 |
| 62 | 210980_s_at | ASAH1 | N-acylsphingosine amidohydrolase (acid | 8p22.p21.3 | Exemplarsequence | Hs.75811.1 | U47674.1 | g3860239 |
| | | | ceramidase) 1 | | | | | |
| 63 | 209997_x_at | PCM1 | pericentriolar material 1 | 8p22-p21.3 | Exemplarsequence | Hs.315478.0 | BC000453.1 | g12653366 |
| 64 | 208231_at | NRG1 | neuregulin 1 | 8p21-p12 | Exemplarsequence | Hs.172816.0 | NM_013960.1 | g7669519 |
| 65 | 205770_at | GSR | glutathione reductase | 8p21.1 | Exemplarsequence | Hs.121524.0 | NM_000637.1 | 910835188 |
| 66 | 217819_at | LOC51125 | HSPC041 protein | 8p11.21 | Exemplarsequence | Hs.7953.0 | NM_016099.1 | g7705820 |
| 67 | 209517_s_at | ASH2L | ash2 (absent, small, or homeotic)-like | 8p11.2 | Exemplarsequence | Hs.6856.1 | AB020982.1 | g4417209 |
| | | | (Drosophila) | | | | | |
| 68 | 216519_s_at | PROSC | proline synthetase co-transcribed homolog | 8p11.2 | Consensussequence | Hs.301959.3 | AK021923.1 | Hs.301959.3.S1 |
| | | | (bacterial) | | | | | |
| 69 | 222551_s_at | FLJ20989 | hypothetical protein FLJ20989 | | Consensussequence | Hs.169615.0 | AI197841 | Hs.169615.0 |
| 70 | 206573_at | KCNQ3 | potassium voltage-gated channel, KQT-like | 8q24 | Exemplarsequence | Hs.40866.0 | NM_004519.1 | g4758629 |
| | | | subfamily, member 3 | | | | | |
| 71 | 225439_at | CML66 | chronic myelogenous leukemia tumor | 8q23 | Consensussequence | Hs.195870.0 | BC000967.2 | Hs.195870.0 |
| | | | antigen 66 | | | | | |
| 72 | 200949_x_at | RPS20 | ribosomal protein S20 | 8q12 | Exemplarsequence | Hs.8102.0 | NM_001023.1 | g4506696 |
| 73 | 222525_s_at | FLJ10853 | hypothetical protein FLJ10853 | 8p21.1 | Consensussequence | Hs.72085.0 | AU160632 | Hs.72085.0 |
| 74 | 222998_at | MAF1 | homolog of yeast MAF1 | 8q24.3 | Exemplarsequence | Hs.324157.0 | AL136937.1 | g12053368 |
| 75 | 218679_s_at | VPS28 | vacuolar protein sorting 28 (yeast) | 8q24.3 | Exemplarsequence | Hs.293441.0 | NM_016208.1 | g7705884 |
| 76 | 223231_at | CDA11 | CDA11 protein | 8q24.1 | Exemplarsequence | Hs.11810.0 | AF212250.1 | g13182774 |
| 77 | 241342_at | LOC157378 | hypothetical protein BC017881 | 8q24.13 | Consensussequence | Hs.187646.0 | BG288115 | Hs.187646.0.A1 |
| 78 | 204278_s_at | EBAG9 | estrogen receptor binding site associated, | 8q23 | Exemplarsequence | Hs.9222.0 | NM_004215.1 | g4758229 |
| | | | antigen, 9 | | | | | |
| 79 | 218059_at | LOC51123 | HSPC038 protein | 8q22.3 | Exemplarsequence | Hs.23528.0 | NM_016096.1 | g7705816 |
| 80 | 201433_s_at | PTDSS1 | phosphatidylserine synthase 1 | 8q22 | Exemplarsequence | Hs.77329.0 | NM_014754.1 | g7662646 |
| 81 | 221925_s_at | FLJ22490 | hypothetical protein FLJ22490 | 8q12.3 | Consensussequence | Hs.153746.1 | BE044503 | Hs.153746.1.S1 |
| 82 | 216246_at | RPS20 | ribosomal protein S20 | 8q12 | Consensussequence | Hs.8102.2 | AF113008.1 | Hs.8102.2 |
| 83 | 224743_at | | Homo sapiens, clone IMAGE: 3897094, | | Consensussequence | Hs.13328.0 | BF965065 | Hs.13328.0_RC |
| | | | mRNA | | | | | |
| 84 | 210950_s_at | FDFT1 | farnesyl-diphosphate farnesyltransferase 1 | 8p23.1-p22 | Exemplarsequence | Hs.48876.0 | BC003573.1 | g13097746 |
| 85 | 209471_s_at | FNTA | farnesyltransferase, CAAX box, alpha | 8p22-q11 | Exemplarsequence | Hs.138381.0 | L00634.1 | g292030 |
| 86 | 225378_at | FLJ32642 | hypothetical protein FLJ32642 | 8p22 | Consensussequence | Hs.101617.0 | AI866426 | Hs.101617.0_RC |
| 87 | 228024_at | FLJ32642 | hypothetical protein FLJ32642 | 8p22 | Consensussequence | Hs.290855.0 | AW028100 | Hs.290855.0 |
| 88 | 201985_at | KIAA0196 | KIAA0196 gene product | 8p22 | Exemplarsequence | Hs.8294.0 | NM_014846.1 | g7661987 |
| 89 | 207000_s_at | PPP3CC | protein phosphatase 3 (formerly 2B), | 8p21.2 | Exemplarsequence | Hs.75206.0 | NM_005605.1 | g5031988 |
| | | | catalytic subunit, gamma isoform | | | | | |
| | | | (calcineurin A gamma) | | | | | |
| 90 | 227075_at | ELP3 | likely ortholog of mouse elongation protein 3 | 8p21.1 | Consensussequence | Hs.267905.2 | AI949204 | Hs.267905.2_RC |
| | | | homolog (S. cerevisiae) | | | | | |
| 91 | 221916_at | NEFL | neurofilament, light polypeptide 68 kDa | 8p21 | Consensussequence | Hs.302689.1 | BF055311 | Hs.302689.1.S1 |
| 92 | 235472_at | FUT10 | fucosyltransferase 10 (alpha (1,3) | 8p11.23 | Consensussequence | Hs.132665.0 | AI147738 | Hs.132665.0.A1 |
| | | | fucosyltransferase) | | | | | |
| 93 | 209384_at | PROSC | proline synthatase co-transcribed homolog | 8p11.2 | Consensussequence | Hs.301959.0 | AA176833 | Hs.301959.0.A2 |
| | | | (bacterial) | | | | | |
| 94 | 218017_s_at | FLJ22242 | hypothetical protein FLJ22242 | 8p11.1 | Exemplarsequence | Hs.288057.0 | NM_025070.1 | g13376612 |
| 95 | 232640_at | HT002 | HT002 protein; hypertension-related calcium- | 8q24-qter | Consensussequence | Hs.238928.2 | AK023070.1 | Hs.238928.2.S1 |
| | | | regulated gene | | | | | |
| 96 | 218858_at | FLJ12428 | hypothetical protein FLJ12428 | 8q24.12 | Exemplarsequence | Hs.87729.0 | NM_022783.1 | g12232472 |
| 97 | 202872_at | ATP6V1C1 | ATPase, H+ transporting. lysosomal 42 kDa, | 8q22.3 | Consensussequence | Hs.86905.0 | NM_001695.1 | Hs.86905.0 |
| | | | V1 subunit C, isoform 1 | | | | | |
| 98 | 209066_x_at | UQCRB | ubiquinol-cytochrome c reductase binding | 8q22 | Exemplarsequence | Hs.131255.1 | M26700.1 | g190803 |
| | | | protein | | | | | |
| 99 | 242338_at | DKFZp762C1112 | hypothetical protein DKFZp762C1112 | 8q21.3 | Consensussequence | Hs.330692.0 | BG535396 | Hs.330692.0.S1 |
| 100 | 218549_s_at | CGI-90 | CGI-90 protein | 8q21.13 | Exemplarsequence | Hs.44222.0 | NM_016033.1 | g7705802 |
| 101 | 201652_at | COPS5 | COP9 constitutive photomorphogenic | 8q12.3 | Exemplarsequence | Hs.198767.0 | NM_006837.1 | g5803045 |
| | | | homolog subunit 5 (Arabidopsis) | | | | | |
| 102 | 226123_at | LOC286180 | hypothetical protein LOC286180 | 8q12.1 | Consensussequence | Hs.60238.0 | AI870918 | Hs.60238.0_RC |
| 103 | 239877_at | MTMR9 | myotubularin related protein 9 | 8p23-p22 | Consensussequence | Hs.128277.0 | AI499833 | Hs.128277.0_RC |
| 104 | 222714_s_at | CGI-83 | CGI-83 protein | 8p22-q22.3 | Exemplarsequence | Hs.118554.0 | BC000878.1 | g12654126 |
| 105 | 239303_at | | ESTs | | Consensussequence | Hs.97814.0 | AA933717 | Hs.97814.0.A1 |
| 106 | 203941_at | FLJ10871 | hypothetical protein FLJ10871 | 8p12 | Exemplarsequence | Hs.15562.0 | NM_018250.1 | g8922725 |
| 107 | 215983_s_at | D8S2298E | reproduction 8 | 8p12-p11.2 | Consensussequence | Hs.153678.1 | D83768.1 | Hs.153678.1.S1 |
| 108 | 208846_s_at | VDAC3 | voltage-dependent anion channel 3 | 8p11.2 | Exemplarsequence | Hs.7381.0 | U90943.1 | g2735306 |
| 109 | 214394_x_at | EEF1D | eukaryotic translation elongation factor 1 | 8q24.3 | Consensussequence | Hs.223241.1 | AI613383 | Hs.223241.1.A1 |
| | | | delta (guanine nucleotide exchange protein) | | | | | |
| 110 | 219189_at | FBXL6 | F-box and leucine-rich repeat protein 6 | 8q24.3 | Exemplarsequence | Hs.12271.0 | NM_024555.1 | g13435140 |
| 111 | 211060_x_at | GPAA1 | GPAA1P anchor attachment protein 1 | 8q24.3 | Exemplarsequence | g13623546 | BC006383.1 | g13623546 |
| | | | homolog (yeast) | | | | | |
| 112 | 212090_at | VPS28 | vacuolar protein sorting 28 (yeast) | 8q24.3 | Consensussequence | Hs.101067.2 | AL571424 | Hs.101067.2 |
| 113 | 227778_at | | Homo sapiens cDNA FLJ35542 fis, clone | | Consensussequence | Hs.26563.0 | H11075 | Hs.26563.0_RC |
| | | | SPLEN2002917. | | | | | |
| 114 | 212556_at | SCRIB | scribble | 8q24.3 | Consensussequence | Hs.239784.0 | AI469403 | Hs.239784.0.S1 |
| 115 | 36936_at | TSTA3 | tissue specific transplantation antigen P35B | 8q24.3 | Consensussequence | 4900667_rc | U58766 | 4900667_rc |
| 116 | 243301_at | | ESTs, Weakly similar to JX0369 collagen | | Consensussequence | Hs.122254.0 | AW241910 | Hs.122254.0.A1 |
| | | | alpha 1(XIX) chain precursor - human | | | | | |
| | | | [H. sapiens] | | | | | |
| 117 | 203113_s_at | EEF1D | eukaryotic translation elongation factor 1 | 8q24.3 | Exemplarsequence | Hs.223241.0 | NM_001960.1 | g4503478 |
| | | | delta (guanine nucleotide exchange protein) | | | | | |
| 118 | 222155_s_at | FLJ11856 | putative G-protein coupled receptor | 8q24.3 | Consensussequence | Hs.6459.1 | AK021918.1 | Hs.6459.1.S1 |
| | | | GPCR41 | | | | | |
| 119 | 221629_x_at | LOC51236 | hypothetical protein LOC51236 | 8q24.3 | Exemplarsequence | Hs.300224.1 | AF151022.1 | g7106765 |
| 120 | 202145_at | LY6E | lymphocyte antigen 6 complex, locus E | 8q24.3 | Exemplarsequence | Hs.77667.0 | NM_002346.1 | g4505048 |
| 121 | 223421_at | MGC13010 | hypothetical protein MGC13010 | 8q24.3 | Exemplarsequence | Hs.332040.0 | BC005073.1 | g13477216 |
| 122 | 225203_at | PPP1R16A | protein phosphatase 1, regulatory (inhibitor) | 8q24.3 | Consensussequence | Hs.12185.0 | AI742931 | Hs.12185.0_RC |
| | | | subunit 16A | | | | | |
| 123 | 222133_s_at | CGI-72 | CGI-72 protein | 8q24.3 | Consensussequence | Hs.288435.0 | AK022280.1 | Hs.288435.0 |
| 124 | 226942_at | FLJ21615 | hypothetical protein FLJ21615 | 8q24.22 | Consensussequence | Hs.44159.0 | AI742668 | Hs.44159.0.S1 |
| 125 | 227523_s_at | CGI-72 | CGI-72 protein | 8q24.3 | Consensussequence | Hs.318725.1 | AA192936 | Hs.318725.1.S1 |
| 126 | 226564_at | ZNF406 | zinc finger protein 406 | 8q24.22 | Consensussequence | Hs.15611.0 | BF941325 | Hs.15611.0 |
| 127 | 224790_at | DDEF1 | development and differentiation enhancing | 8q24.1-q24.2 | Consensussequence | Hs.10669.1 | W03103 | Hs.10669.1.S2 |
| | | | factor 1 | | | | | |
| 128 | 224796_at | DDEF1 | development and differentiation enhancing | 8q24.1-q24.2 | Consensussequence | Hs.10669.1 | W03103 | Hs.10669.1.S2 |
| | | | factor 1 | | | | | |
| 129 | 226536_at | FLJ32440 | hypothetical protein FLJ32440 | 8q24.13 | Consensussequence | Hs.58609.0 | AL562908 | Hs.58609.0.S1 |
| 130 | 241370_at | | Homo sapiens cDNA FLJ37785 fis, clone | | Consensussequence | Hs.100691.0 | AA278233 | Hs.100691.0_RC |
| | | | BRHIP2028330. | | | | | |
| 131 | 214061_at | MGC21654 | unknown MGC21654 product | 8q24.13 | Consensussequence | Hs.95631.0 | AI017564 | Hs.95631.0 |
| 132 | 222543_at | PRO2577 | hypothetical protein PRO2577 | 8q24.13 | Consensussequence | Hs.241576.0 | AF131854.1 | Hs.241576.0.S2 |
| 133 | 225864_at | | Homo sapiens cDNA FLJ23705 fis, clone | | Consensussequence | Hs.49136.0 | AL039862 | Hs.49136.0.A1 |
| | | | HEP11066. | | | | | |
| 134 | 235266_at | PRO2000 | PRO2000 protein | 8q24.13 | Consensussequence | Hs.222088.0 | AI139629 | Hs.222088.0_RC |
| 135 | 203556_at | KIAA0854 | KIAA0854 protein | 8q24.13 | Exemplarsequence | Hs.30209.0 | NM_014943.1 | g7662341 |
| 136 | 202431_s_at | MYC | v-myc myelocytomatosis viral oncogene | 8q24.12-q24.13 | Exemplarsequence | Hs.79070.0 | NM_002467.1 | g12962934 |
| | | | homolog (avian) | | | | | |
| 137 | 208608_s_at | SNTB1 | syntrophin, beta 1 (dystrophin-associated | 8q23-q24 | Exemplarsequence | Hs.95011.0 | NM_021021.1 | g11321639 |
| | | | protein A1, 59 kDa, basic component 1) | | | | | |
| 138 | 226438_at | | Homo sapiens cDNA: FLJ21447 fis, clone | | Consensussequence | Hs.44241.0 | AK025100.1 | Hs.44241.0 |
| | | | COL04468. | | | | | |
| 139 | 218482_at | DC6 | DC6 protein | 8q23.2 | Exemplarsequence | Hs.283740.0 | NM_020189.1 | g9910185 |
| 140 | 204274_at | EBAG9 | estrogen receptor binding site associated, | 8q23 | Consensussequence | Hs.9222.0 | AA812215 | Hs.9222.0 |
| | | | antigen, 9 | | | | | |
| 141 | 218197_s_at | OXR1 | oxidation resistance 1 | 8q23 | Exemplarsequence | Hs.169111.0 | NM_018002.1 | g8922240 |
| 142 | 221266_s_at | DCSTAMP | DC-specific transmembrane protein | 8q22 | Exemplarsequence | g13540564 | NM_030788.1 | g13540564 |
| 143 | 222553_x_at | OXR1 | oxidation resistance 1 | 8q23 | Consensussequence | Hs.169111.0 | AL541048 | Hs.169111.0.S2 |
| 144 | 223879_s_at | OXR1 | oxidation resistance 1 | 8q23 | Exemplarsequence | Hs.169111.1 | AF309387.1 | g10954045 |
| 145 | 235429_at | EIF3S6 | eukaryotic translation initiation factor 3, | 8q22-q23 | Consensussequence | Hs.161623.0 | AW965494 | Hs.161623.0.A1 |
| | | | subunit 6 48 kDa | | | | | |
| 146 | 226463_at | | Homo sapiens cDNA FLJ33383 fis, clone | | Consensussequence | Hs.290880.0 | AW241758 | Hs.290880.0.A1 |
| | | | BRACE2006514. | | | | | |
| 147 | 202635_s_at | POLR2K | polymerase (RNA) II (DNA directed) | 8q22.2 | Exemplarsequence | Hs.150675.0 | NM_005034.1 | g4826923 |
| | | | polypeptide K, 7.0 kDa | | | | | |
| 148 | 202874_s_at | ATP6V1C1 | ATPase, H⁺ transporting, lysosomal 42 kDa, | 8q22.3 | Exemplarsequence | Hs.86905.0 | NM_001695.1 | g4502314 |
| | | | V1 subunit C, isoform 1 | | | | | |
| 149 | 231784_s_at | DKFZP564O0463 | DKFZP564O0463 protein | 8q22.3 | Consensussequence | Hs.273344.2 | AK001874.1 | Hs.273344.2 |
| 150 | 208454_s_at | PGCP | plasma glutamate carboxypeptidase | 8q22.2 | Exemplarsequence | Hs.278993.0 | NM_016134.1 | g7706386 |
| 151 | 223110_at | DKFZP434I116 | DKFZP434I116 protein | 8q22.1 | Exemplarsequence | Hs.16621.0 | BC003701.1 | g13277583 |
| 152 | 218905_at | FLJ20530 | hypothetical protein FLJ20530 | 8q22.1 | Exemplarsequence | Hs.279521.0 | NM_017864.1 | g8923495 |
| 153 | 225600_at | | Homo sapiens cDNA FLJ40637 fis, clone | | Consensussequence | Hs.6390.1 | AW303300 | Hs.6390.1.A1 |
| | | | THYMU2015984. | | | | | |
| 154 | 225603_s_at | | Homo sapiens cDNA FLJ40637 fis, clone | | Consensussequence | Hs.6390.1 | AW303300 | Hs.6390.1.A1 |
| | | | THYMU2015984. | | | | | |
| 155 | 212251_at | | Homo sapiens, Similar to LYRIC, clone | | Consensussequence | Hs.243901.0 | AI972475 | Hs.243901.0.S1 |
| | | | MGC: 41931 IMAGE: 5298467, mRNA, | | | | | |
| | | | complete cds | | | | | |
| 156 | 222699_s_at | FLJ13187 | phafin 2 | 8q22.1 | Consensussequence | Hs.29724.0 | BF439250 | Hs.29724.0.S1 |
| 157 | 243927_x_at | | ESTs, Highly similar to DKFZP434I116 | | Consensussequence | Hs.50273.0 | AI636247 | Hs.50273.0_RC |
| | | | protein [Homo sapiens] [H. sapiens] | | | | | |
| 158 | 225599_s_at | | Homo sapiens cDNA FLJ40637 fis, clone | | Consensussequence | Hs.6390.1 | AW303300 | Hs.6390.1.A1 |
| | | | THYMU2015984. | | | | | |
| 159 | 226721_at | | Homo sapiens, clone IMAGE: 5314143, | | Consensussequence | Hs.165539.0 | AW517711 | Hs.165539.0_RC |
| | | | mRNA | | | | | |
| 160 | 203790_s_at | UK114 | translational inhibitor protein p14.5 | 8q22 | Consensussequence | Hs.18426.0 | N54448 | Hs.18426.0.S1 |
| 161 | 209065_at | UQCRB | ubiquinol-cytochrome c reductase binding | 8q22 | Exemplarsequence | Hs.131255.1 | BC005230.1 | g13528857 |
| | | | protein | | | | | |
| 162 | 223085_at | RNF19 | ring finger protein 19 | 8q22 | Exemplarsequence | Hs.48320.1 | AB029316.1 | g13366023 |
| 163 | 202118_s_at | CPNE3 | copine III | 8q21.13 | Consensussequence | Hs.14158.0 | AA541758 | Hs.14158.0 |
| 164 | 210296_s_at | PXMP3 | perosisomal membrane protein 3, 35 kDa | 8q21.1 | Exemplarsequence | Hs.180612.1 | BC005375.1 | g13529226 |
| | | | (Zellweger syndrome) | | | | | |
| 165 | 202905_x_at | NBS1 | Nijmegen breakage syndrome 1 (nibrin) | 8q21-q24 | Consensussequence | Hs.25812.0 | AI796269 | Hs.25812.0.S1 |
| 166 | 202907_s_at | NBS1 | Nijmegen breakage syndrome 1 (nibrin) | 8q21-q24 | Exemplarsequence | Hs.25812.0 | NM_002485.2 | g6996019 |
| 167 | 208166_at | MMP16 | matrix metalloproteinase 16 (membrane- | 8q21 | Exemplarsequence | Hs.90800.0 | NM_022564.1 | g13027799 |
| | | | inserted) | | | | | |
| 168 | 202906_s_at | NBS1 | Nijmegen breakage syndrome 1 (nibrin) | 8q21-q24 | Consensussequence | Hs.25812.0 | AI796269 | Hs.25812.0.S1 |
| 169 | 217299_s_at | NBS1 | Nijmegen breakage syndrome 1 (nibrin) | 8q21-q24 | Consensussequence | Hs.25812.1 | AK001017.1 | Hs.25812.1.S1 |
| 170 | 204226_at | STAU2 | staufen, RNA binding protein, homolog 2 | 8q13-q21.1 | Exemplarsequence | Hs.96870.0 | NM_014393.1 | g7657624 |
| | | | (Drosophila) | | | | | |
| 171 | 219449_s_at | FLJ20533 | hypothetical protein FLJ20533 | 8q13.3 | Exemplarsequence | Hs.106650.0 | NM_017866.1 | g8923499 |
| 172 | 226083_at | FLJ20533 | hypothetical protein FLJ20533 | 8q13.3 | Consensussequence | Hs.106650.1 | AA886902 | Hs.106650.1_RC |
| 173 | 226269_at | | Homo sapiens mRNA; cDNA | | Consensussequence | Hs.168950.0 | AL110252.1 | Hs.168950.0 |
| | | | DKFZp566A1046 (from clone | | | | | |
| | | | DKFZp566A1046) | | | | | |
| 174 | 201399_s_at | TRAM | translocating chain-associating membrane | 8q13.1 | Exemplarsequence | Hs.4147.0 | NM_014294.1 | g7657654 |
| | | | protein | | | | | |
| 175 | 202956_at | BIG1 | brefeldin A-inhibited guanine nucleotide- | 8q13 | Exemplarsequence | Hs.94631.0 | NM_006421.2 | g6715588 |
| | | | exchange protein 1 | | | | | |
| 176 | 216266_s_at | BIG1 | brefeldin A-inhibited guanine nucleotide- | 8q13 | Consensussequence | Hs.94631.1 | AK025637.1 | Hs.94631.1.S1 |
| | | | exchange protein 1 | | | | | |
| 177 | 224046_s_at | PDE7A | phosphodiesterase 7A | 8q13 | Exemplarsequence | Hs.150395.1 | U67932.1 | g2306763 |
| 178 | 232314_at | | Homo sapiens cDNA FLJ14270 fis, clone | | Consensussequence | Hs.97603.0 | AU156769 | Hs.97603.0 |
| | | | PLACE1004491. | | | | | |
| 179 | 232149_s_at | | Homo sapiens cDNA FLJ11963 fis, clone | | Consensussequence | Hs.306621.0 | BF056507 | Hs.306621.0 |
| | | | HEMBB1001051. | | | | | |
| 180 | 220038_at | SGKL | serum/glucocorticoid regulated kinase-like | 8q12.3-8q13.1 | Exemplarsequence | Hs.279696.0 | NM_013257.1 | g7019526 |
| 181 | 227627_at | SGKL | serum/glucocorticoid regulated kinase-like | 8q12.3-8q13.1 | Consensussequence | Hs.24131.0 | AV690866 | Hs.24131.0.S1 |
| 182 | 218185_s_at | FLJ10511 | hypothetical protein FLJ10511 | 8q12.2 | Exemplarsequence | Hs.106768.0 | NM_018120.1 | g8922478 |
| 183 | 222550_at | FLJ10511 | hypothetical protein FLJ10511 | 8q12.2 | Consensussequence | Hs.106768.0 | AK024053.1 | Hs.106768.0 |
| 184 | 225008_at | MGC34646 | hypothetical protein MGC34646 | 8q12.1 | Consensussequence | Hs.332422.0 | AF339775.1 | Hs.332422.0 |
| 185 | 210896_s_at | ASPH | aspartate beta-hydroxylase | 8q12.1 | Exemplarsequence | Hs.283664.4 | AF306765.1 | g11991236 |
| 186 | 208731_at | RAB2 | RAB2, member RAS oncogene family | 8q12.1 | Consensussequence | Hs.78305.0 | NM_002865.1 | Hs.78305.0_RC |
| 187 | 208734_x_at | RAB2 | RAB2, member RAS oncogene family | 8q12.1 | Exemplarsequence | Hs.78305.0 | M28213.1 | g550061 |
| 188 | 222701_s_at | MGC2217 | hypothetical protein MGC2217 | 8q11.23 | Consensussequence | Hs.323164.0 | AA570393 | Hs.323164.0 |
| 189 | 226119_at | LOC115294 | similar to hypothetical protein FLJ10883 | 8q11.22 | Consensussequence | Hs.60293.0 | AA453163 | Hs.60293.0_RC |
| 190 | 235507_at | LOC115294 | similar to hypothetical protein FLJ10883 | 8q11.22 | Consensussequence | Hs.99580.0 | AA461195 | Hs.99580.0.A1 |
| 191 | 209096_at | UBE2V2 | ubiquitin-conjugating enzyme E2 variant 2 | 8q11.1 | Exemplarsequence | Hs.79300.0 | U62136.2 | g4775663 |
| 192 | 207529_at | DEFA5 | defensin, alpha 5, Paneth cell-specific | 8pter-p21 | Exemplarsequence | Hs.72887.0 | NM_021010.1 | g10337584 |
| 193 | 219281_at | MSRA | methionine sulfoxide reductase A | 8p23.1 | Exemplarsequence | Hs.26458.0 | NM_012331.2 | g13259538 |
| 194 | 213457_at | | Homo sapiens cDNA FLJ39185 fis, clone | | Consensussequence | Hs.24724.1 | BF739959 | Hs.24724.1.A2 |
| | | | OCBBF2004418. | | | | | |
| 195 | 225478_at | | Homo sapiens cDNA FLJ39185 fis, clone | | Consensussequence | Hs.24724.0 | BE783723 | Hs.24724.0 |
| | | | OCBBF2004418. | | | | | |
| 196 | 221504_s_at | ATP6V1H | ATPase, H+ transporting, lysosomal | 8p22-q22.3 | Exemplarsequence | Hs.19575.0 | AF112204.1 | g6563195 |
| | | | 50/57 kDa, V1 subunit H | | | | | |
| 197 | 200762_at | DPYSL2 | dihydropyrimidinase-like 2 | 8p22-p21 | Exemplarsequence | Hs.173381.0 | NM_001386.1 | g4503376 |
| 198 | 200839_s_at | CTSB | cathepsin B | 8p22 | Exemplarsequence | Hs.297939.0 | NM_001908.1 | g4503138 |
| 199 | 225187_at | DBC-1 | p30 DBC protein | 8p22 | Consensussequence | Hs.181102.2 | BC003172.1 | Hs.181102.2 |
| 200 | 210762_s_at | DLC1 | deleted in liver cancer 1 | 8p22-p21.3 | Exemplarsequence | Hs.8700.0 | AF026219.1 | g2559001 |
| 201 | 224822_at | DLC1 | deleted in liver cancer 1 | 8p22-p21.3 | Consensussequence | Hs.8700.1 | AA524250 | Hs.8700.1.S2 |
| 202 | 222730_s_at | ZDHHC2 | zinc finger, DHHC domain containing 2 | 8p21.3-p22 | Consensussequence | Hs.5943.0 | AI814257 | Hs.5943.0.S1 |
| 203 | 222731_at | ZDHHC2 | zinc finger, DHHC domain containing 2 | 8p21.3-p22 | Consensussequence | Hs.5943.0 | AI814257 | Hs.5943.0.S1 |
| 204 | 212866_at | | Homo sapiens, similar to hypothetical | | Consensussequence | Hs.300861.0 | AI081543 | Hs.300861.0 |
| | | | protein DKFZp564N123.1 - human | | | | | |
| | | | (fragment), clone IMAGE: 5220614, mRNA | | | | | |
| 205 | 212110_at | KIAA0062 | KIAA0062 protein | 8p21.2 | Consensussequence | Hs.89868.0 | D31887.1 | Hs.89868.0_RC |
| 206 | 220686_s_at | PIWIL2 | piwi-like 2 (Drosophila) | 8p21.2 | Exemplarsequence | Hs.274150.0 | NM_018068.1 | g8922369 |
| 207 | 221094_s_at | ELP3 | likely ortholog of mouse elongation protein 3 | 8p21.1 | Exemplarsequence | Hs.267905.0 | NM_018091.1 | g8922417 |
| | | | homolog (S. cerevisiae) | | | | | |
| 208 | 225609_at | GSR | glutathione reductase | 8p21.1 | Consensussequence | Hs.193974.0 | AI888037 | Hs.193974.0.S1 |
| 209 | 227102_at | TRIM35 | tripartite motif-containing 35 | 8p21.1 | Consensussequence | Hs.137732.0 | AA115933 | Hs.137732.0 |
| 210 | 241252_at | LOC157570 | hypothetical protein LOC157570 | 8p21.1 | Consensussequence | Hs.99480.1 | AI732824 | Hs.99480.1_RC |
| 211 | 218955_at | BRF2 | BRF2, subunit of RNA polymerase III | 8p11.1 | Exemplarsequence | Hs.274136.0 | NM_018310.1 | g8922843 |
| | | | transcription initiation factor, BRF1-like | | | | | |
| 212 | 228189_at | BAG4 | BCL2-associated athanogene 4 | 8p11.21 | Consensussequence | Hs.7859.0 | T32429 | Hs.7859.0.A1 |
| 213 | 223568_s_at | HTPAP | HTPAP protein | 8p11.21 | Exemplarsequence | Hs.169341.0 | AF212238.1 | g13182756 |
| 214 | 226384_at | HTPAP | HTPAP protein | 8p11.21 | Consensussequence | Hs.8841.0 | BE858787 | Hs.8841.0.A1 |
| 215 | 220985_s_at | DKFZP564A022 | hypothetical protein DKFZp564A022 | 8p11.1 | Exemplarsequence | g13569925 | NM_030954.1 | g13569925 |
| 216 | 224776_at | DKFZp586M1819 | hypothetical protein DKFZp586M1819 | 8p11.1 | Consensussequence | Hs.25664.1 | BF513102 | Hs.25664.1.A1 |
| 217 | 226104_at | DKFZP564A022 | hypothetical protein DKFZp564A022 | 8p11.1 | Consensussequence | Hs.22629.0 | AI052736 | Hs.22629.0_RC |
| 218 | 221542_s_at | C8orf2 | chromosome 8 open reading frame 2 | 8p11.2 | Consensussequence | Hs.125849.0 | T90773 | Hs.125849.0 |
| 219 | 221543_s_at | C8orf2 | chromosome 8 open reading frame 2 | 8p11.2 | Exemplarsequence | Hs.125849.0 | AL442077.1 | g10241715 |
| 220 | 209341_s_at | IKBKB | inhibitor of kappa light polypeptide gene | 8p11.2 | Consensussequence | Hs.226573.0 | AU153366 | Hs.226573.0 |
| | | | enhancer in B-cells, kinase beta | | | | | |
| 221 | 209385_s_at | PROSC | proline synthetase co-transcribed homolog | 8p11.2 | Exemplarsequence | Hs.301959.0 | AL136616.1 | g12052757 |
| | | | (bacterial) | | | | | |
| 222 | 207859_s_at | CHRNB3 | cholinergic receptor, nicotinic, beta | 8p11.2 | Exemplarsequence | Hs.96094.0 | NM_000749.1 | g4502834 |
| | | | polypeptide 3 | | | | | |
| 223 | 209342_s_at | IKBKB | inhibitor of kappa light polypeptide gene | 8p11.2 | Exemplarsequence | Hs.226573.0 | AF080158.1 | g4185274 |
| | | | enhancer in B-cells, kinase beta | | | | | |
| 224 | 228790_at | MGC39325 | hypothetical protein MGC39325 | 8q11.23 | Consensussequence | Hs.74280.0 | AW264082 | Hs.74280.0 |
| 225 | 222536_s_at | DKFZp434K1210 | hypothetical protein DKFZp434K1210 | 8p21.1 | Consensussequence | Hs.32352.0 | N36098 | Hs.32352.0.S1 |
| 226 | 229139_at | | Homo sapiens, clone IMAGE: 4245141, | | Consensussequence | Hs.293836.0 | AI202201 | Hs.293836.0.A1 |
| | | | mRNA | | | | | |
| 227 | 207013_s_at | MMP16 | matrix metalloproteinase 16 (membrane- | 8q21 | Exemplarsequence | Hs.90800.1 | AB009303.1 | g2662305 |
| | | | inserted) | | | | | |
| 228 | 219416_at | SCARA3 | scavenger receptor class A, member 3 | 8p21 | Exemplarsequence | Hs.128856.0 | NM_016240.1 | g7705335 |

| # | Sequence Source | Unigene_Accession | Cluster_Type | LocusLink | Full_Length_Reference_Seq |
|---|---|---|---|---|---|
| 1 | GenBank | Hs.25590 | fulllength | 6781 | NM_003155; stanniocalcin 1 |
| 2 | GenBank | Hs.16292 | est | | |
| 3 | GenBank | Hs.42474 | fulllength | 9242 | NM_005098; musculin (activated B-cell factor-1) |
| 4 | RefSeq | Hs.119684 | fulllength | 8794 | NM_003841; tumor necrosis factor receptor superfamily, member 10c precursor |
| 5 | GenBank | Hs.52931 | fulllength | 148 | NM_000680; alpha-1A-adrenergic receptor isoform 1 NM_033302; alpha- |
| | | | | | 1A-adrenergic receptor isoform 3 NM_033303; alpha-1A-adrenergic |
| | | | | | receptor isoform 2 NM_033304; alpha-1A-adrenergic receptor isoform 4 |
| 6 | RefSeq | Hs.289063 | fulllength | 80346 | NM_025232; hypothetical protein FLJ22246 |
| 7 | GenBank | Hs.172685 | fulllength | 23039 | NM_015024; exportin 7 |
| 8 | GenBank | Hs.380963 | fulllength | 29883 | NM_013354; CCR4-NOT transcription complex, subunit 7 isoform 1 |
| | | | | | NM_054026; CCR4-NOT transcription complex, subunit 7 isoform 2 |
| 9 | GenBank | Hs.375560 | fulllength | 286057 | NM_173686; hypothetical protein FLJ34715 |
| 10 | GenBank | Hs.7471 | fulllength | 83877 | NM_031940; BBP-like protein 1 isoform b NM_078473; BBP-like protein 1 isoform a |
| 11 | RefSeq | Hs.131255 | fulllength | 7381 | NM_006294; ubiquinol-cytochrome c reductase binding protein |
| 12 | GenBank | Hs.41185 | fulllength | 157869 | NM_153225; hypothetical protein FLJ40021 |
| 13 | GenBank | Hs.167011 | | | |
| 14 | GenBank | Hs.44222 | fulllength | 51115 | NM_016033; CGI-90 protein |
| 15 | GenBank | Hs.278634 | | 23514 | |
| 16 | GenBank | Hs.79300 | fulllength | 7336 | NM_003350; ubiquitin-conjugating enzyme E2 variant 2 |
| 17 | RefSeq | Hs.288716 | fulllength | 80185 | NM_025115; hypothetical protein FLJ23263 |
| 18 | RefSeq | Hs.404119 | fulllength | 7264 | NM_003313; tissue specific transplantation antigen P35B |
| 19 | GenBank | Hs.4147 | fulllength | 23471 | NM_014294; translocating chain-associating membrane protein |
| 20 | GenBank | Hs.98471 | est | 137682 | NM_152416; hypothetical protein MGC40214 |
| 21 | RefSeq | Hs.170198 | fulllength | 9650 | NM_014637; KIAA0009 gene product |
| 22 | RefSeq | Hs.169395 | fulllength | 79870 | NM_024812; brain and acute leukemia, cytoplasmic |
| 23 | GenBank | Hs.55043 | | | |
| 24 | GenBank | Hs.27410 | fulllength | 55893 | NM_018660; papillomavirus regulatory factor PRF-1 |
| 25 | GenBank | Hs.170198 | fulllength | 9650 | NM_014637; KIAA0009 gene product |
| 26 | GenBank | Hs.243901 | fulllength | | |
| 27 | RefSeq | Hs.77100 | fulllength | 2961 | NM_002095; general transcription factor IIE, polypeptide 2, beta 34 kDa |
| 28 | GenBank | Hs.75106 | fulllength | 1191 | NM_001831; clusterin |
| 29 | GenBank | Hs.75106 | fulllength | 1191 | NM_001831; clusterin |
| 30 | RefSeq | Hs.3815 | fulllength | 81551 | NM_030795; stathmin-like-protein RB3 |
| 31 | GenBank | Hs.748 | fulllength | 2260 | NM_000604; fibroblast growth factor receptor 1 isoform 1 precursor |
| | | | | | NM_015850; fibroblast growth factor receptor 1 isoform 2 precursor |
| | | | | | NM_023105; fibroblast growth factor receptor 1 isoform 3 precursor |
| | | | | | NM_023106; fibroblast growth factor receptor 1 isoform 4 precursor |
| | | | | | NM_023107; fibroblast growth factor receptor 1 isoform 5 precursor |
| | | | | | NM_023108; fibroblast growth factor receptor 1 isoform 6 precursor |
| | | | | | NM_023109; fibroblast growth factor receptor 1 isoform 7 precursor |
| | | | | | NM_023110; fibroblast growth factor receptor 1 isoform 8 precursor |
| | | | | | NM_023111; fibroblast growth factor receptor 1 isoform 9 precursor |
| 32 | RefSeq | Hs.27721 | fulllength | 54904 | NM_017778; WHSC1L1 protein isoform short NM_023034; WHSC1L1 |
| | | | | | protein isoform long |
| 33 | GenBank | Hs.273344 | fulllength | 25879 | NM_014156; NM_015420; DKFZP564O0463 protein |
| 34 | RefSeq | Hs.169615 | fulllength | 65265 | NM_023080; hypothetical protein FLJ20989 |
| 35 | GenBank | Hs.406141 | fulllength | 137695 | NM_152417; hypothetical protein FLJ32370 |
| 36 | GenBank | Hs.48876 | fulllength | 2222 | NM_004462; farnesyl-diphosphate farnesyltransferase 1 |
| 37 | GenBank | Hs.356463 | fulllength | 2339 | NM_002027; farnesyltransferase, CAAX box, alpha |
| 38 | RefSeq | Hs.380963 | fulllength | 29883 | NM_013354; CCR4-NOT transcription complex, subunit 7 isoform 1 |
| | | | | | NM_054026; CCR4-NOT transcription complex, subunit 7 isoform 2 |
| 39 | RefSeq | Hs.75737 | fulllength | 5108 | NM_006197; pericentriolar material 1 |
| 40 | GenBank | Hs.75737 | fulllength | 5108 | NM_006197; pericentriolar material 1 |
| 41 | GenBank | Hs.75737 | fulllength | 5108 | NM_006197; pericentriolar material 1 |
| 42 | GenBank | Hs.27721 | fulllength | 54904 | NM_017778; WHSC1L1 protein isoform short NM_023034; WHSC1L1 |
| | | | | | protein isoform long |
| 43 | RefSeq | Hs.55097 | fulllength | 28957 | NM_014018; mitochondrial ribosomal protein S28 |
| 44 | GenBank | Hs.840 | fulllength | 3620 | NM_002164; indoleamine-pyrrole 2,3 dioxygenase |
| 45 | RefSeq | Hs.4742 | fulllength | 8733 | NM_003801; anchor attachment protein 1 |
| 46 | RefSeq | Hs.178551 | fulllength | 6132 | NM_000973; ribosomal protein L8 NM_033301; ribosomal protein L8 |
| 47 | GenBank | Hs.331601 | fulllength | 157542 | |
| 48 | GenBank | Hs.343589 | fulllength | 54512 | NM_019037; exosome complex exonuclease RRP41 |
| 49 | GenBank | Hs.10669 | fulllength | 50807 | |
| 50 | GenBank | Hs.86970 | est | | |
| 51 | RefSeq | Hs.23019 | fulllength | 7564 | NM_006958; zinc finger protein 16 (KOX 9) |
| 52 | GenBank | Hs.243901 | fulllength | | |
| 53 | GenBank | Hs.33074 | | | |
| 54 | GenBank | Hs.403869 | fulllength | 137392 | NM_145269; similar to CG6405 gene product |
| 55 | RefSeq | Hs.14158 | fulllength | 8895 | NM_003909; copine III |
| 56 | RefSeq | Hs.99519 | fulllength | 79752 | NM_024699; hypothetical protein FLJ14007 |
| 57 | Refseq | Hs.1276 | fulllength | 661 | NM_001722; RNA polymerase III 53 kDa subunit RPC4 |
| 58 | GenBank | Hs.182364 | fulllength | 83690 | NM_031461; CocoaCrisp |
| 59 | GenBank | Hs.94631 | fulllength | 10565 | NM_006421; brefeldin A-inhibited guanine nucleotide-exchange protein 1 |
| 60 | GenBank | Hs.194562 | fulllength | 7013 | NM_003218; telomeric repeat binding factor 1 isoform 2 NM_017489; |
| | | | | | telomeric repeat binding factor 1 isoform 1 |
| 61 | RefSeq | Hs.5333 | fulllength | 9920 | NM_014867; KIAA0711 gene product |
| 62 | GenBank | Hs.75811 | fulllength | 427 | NM_004315; N-acylsphingosine amidohydrolase (acid ceramidase) 1 |
| | | | | | isoform b NM_177924; N-acylsphingosine amidohydralase (acid |
| | | | | | ceramidase) 1 preproprotein isoform a |
| 63 | GenBank | Hs.75737 | fulllength | 5108 | NM_006197; pericentriolar material 1 |
| 64 | RefSeq | Hs.172816 | fulllength | 3084 | NM_004495; neuregulin 1 isoform HRG-gamma NM_013956; neuregulin |
| | | | | | 1 isoform NRG-beta1 NM_013957; neuregulin 1 isoform HRG-beta2 |
| | | | | | NM_013958; neuregulin 1 isoform HRG-beta3 NM_013959; neuregulin 1 |
| | | | | | isoform SMDF NM_013960; neuregulin 1 isoform ndf43 NM_013961; |
| | | | | | neuregulin 1 isoform GGF NM_013962; neuregulin 1 isoform GGF2 |
| | | | | | NM_013964; neuregulin 1 isoform HRG-alpha |
| 65 | RefSeq | Hs.193974 | fulllength | 2936 | NM_000637; glutathione reductase |
| 66 | RefSeq | Hs.7953 | fulllength | 51125 | NM_016099; NSPC041 protein |
| 67 | GenBank | Hs.6856 | fulllength | 9070 | NM_004674; ash2 (absent, small, or homeotic)-like |
| 68 | GenBank | Hs.301959 | fulllength | 11212 | NM_007198; proline synthetase co-transcribed homolog |
| 69 | GenBank | Hs.169615 | fulllength | 65265 | NM_023080; hypothetical protein FLJ20989 |
| 70 | RefSeq | Hs.40866 | fulllength | 3786 | NM_004519; potassium voltage-gated channel KQT-like protein 3 |
| 71 | GenBank | Hs.195870 | fulllength | 84955 | NM_032869; chronic myelogenous leukemia tumor antigen 66 |
| 72 | RefSeq | Hs.8102 | fulllength | 6224 | NM_001023; ribosomal protein S20 |
| 73 | GenBank | Hs.72085 | fulllength | 55246 | NM_018246; hypothetical protein FLJ10853 |
| 74 | GenBank | Hs.19673 | fulllength | 84232 | NM_032272; homolog of yeast MAF1 |
| 75 | RefSeq | Hs.339697 | fulllength | 51160 | NM_016208; VPS28 protein |
| 76 | GenBank | Hs.11810 | fulllength | 83940 | NM_032026; CDA11 protein |
| 77 | GenBank | Hs.187646 | fulllength | 157378 | |
| 78 | RefSeq | Hs.9222 | fulllength | 9166 | NM_004215; estrogen receptor binding site associated antigen 9 |
| 79 | RefSeq | Hs.23528 | fulllength | 51123 | NM_016096; HSPC038 protein |
| 80 | RefSeq | Hs.77329 | fulllength | 9791 | NM_014754; phosphatidylserine synthase 1 |
| 81 | GenBank | Hs.153746 | fulllength | 79848 | NM_024790; hypothetical protein FLJ22490 |
| 82 | GenBank | Hs.8102 | fulllength | 6224 | NM_001023; ribosomal protein S20 |
| 83 | GenBank | Hs.416904 | | | |
| 84 | GenBank | Hs.48876 | fulllength | 2222 | NM_004462; farnesyl-diphosphate farnesyltransferase 1 |
| 85 | GenBank | Hs.356463 | fulllength | 2339 | NM_002027; farnesyltransferase, CAAX box, alpha |
| 86 | GenBank | Hs.101617 | fulllength | 137492 | NM_152415; hypothetical protein FLJ32642 |
| 87 | GenBank | Hs.101617 | fulllength | 137492 | NM_152415; hypothetical protein FLJ32642 |
| 88 | RefSeq | Hs.8294 | fulllength | 9897 | NM_014846; KIAA0196 gene product |
| 89 | RefSeq | Hs.75206 | fulllength | 5533 | NM_005605; protein phosphatase 3 (formerly 2B), catalytic subunit, |
| | | | | | gamma isoform (calcineurin A gamma) |
| 90 | GenBank | Hs.267905 | fulllength | 55140 | NM_018091; elongation protein 3 homolog |
| 91 | GenBank | Hs.211584 | fulllength | 4747 | NM_006158; neurofilament, light polypeptide 68 kDa |
| 92 | GenBank | Hs.326744 | fulllength | 84750 | NM_032664; fucosyltransferase 10 |
| 93 | GenBank | Hs.301959 | fulllength | 11212 | NM_007198; proline synthetase co-transcribed homolog |
| 94 | RefSeq | Hs.288057 | fulllength | 80140 | NM_025070; hypothetical protein FLJ22242 |
| 95 | GenBank | Hs.238928 | fulllength | 28991 | NM_014066; hypertension-related calcium-regulated gene |
| 96 | RefSeq | Hs.87729 | fulllength | 64798 | NM_022783; hypothetical protein FLJ12428 |
| 97 | GenBank | Hs.86905 | fulllength | 528 | NM_001695; ATPase, H+ transporting, lysosomal 42 kD, V1 subunit C, |
| | | | | | isoform 1 |
| 98 | GenBank | Hs.131255 | fulllength | 7381 | NM_006294; ubiquinol-cytochrome c reductase binding protein |
| 99 | GenBank | Hs.88594 | | 169200 | |
| 100 | RefSeq | Hs.44222 | fulllength | 51115 | NM_016033; CGI-90 protein |
| 101 | RefSeq | Hs.380969 | fulllength | 10987 | NM_006837; COP9 constitutive photomorphogenic homolog subunit 5 |
| 102 | GenBank | Hs.397426 | | 286180 | |
| 103 | GenBank | Hs.48802 | fulllength | 66036 | NM_015458; myotubularin related protein 9 |
| 104 | GenBank | Hs.118554 | fulllength | 51110 | NM_016027; lactamase, beta 2 |
| 105 | GenBank | Hs.446548 | est | | |
| 106 | RefSeq | Hs.15562 | fulllength | 55756 | NM_018250; hypothetical protein FLJ10871 |
| 107 | GenBank | Hs.153678 | fulllength | 7993 | NM_005671; reproduction 8 |
| 108 | GenBank | Hs.7381 | fulllength | 7419 | NM_005662; voltage-dependent anion channel 3 |
| 109 | GenBank | Hs.334798 | fulllength | 1936 | NM_001960; eukaryotic translation elongation factor 1 delta isoform 2 |
| | | | | | NM_032378; eukaryotic translation elongation factor 1 delta isoform 1 |
| 110 | RefSeq | Hs.12271 | fulllength | 26233 | NM_012162; F-box and leucine-rich repeat protein 6 isoform 1 |
| | | | | | NM_024555; F-box and leucine-rich repeat protein 6 isoform 2 |
| 111 | GenBank | Hs.4742 | fulllength | 8733 | NM_003801; anchor attachment protein 1 |
| 112 | GenBank | Hs.339697 | fulllength | 51160 | NM_016208; VPS28 protein |
| 113 | GenBank | Hs.376544 | | | |
| 114 | GenBank | Hs.239784 | est | 23513 | NM_015356; scribble |
| 115 | GenBank | Hs.404119 | fulllength | 7264 | NM_003313; tissue specific transplantation antigen P35B |
| 116 | GenBank | Hs.122254 | est | | |
| 117 | RefSeq | Hs.334798 | fulllength | 1936 | NM_001960; eukaryotic translation elongation factor 1 delta isoform 2 |
| | | | | | NM_032378; eukaryotic translation elongation factor 1 delta isoform 1 |
| 118 | GenBank | Hs.6459 | fulllength | 79581 | NM_024531; putative G-protein coupled receptor GPCR41 |
| 119 | GenBank | Hs.300224 | fulllength | 51236 | NM_016458; brain protein 16 |
| 120 | RefSeq | Hs.77667 | fulllength | 4061 | NM_002346; lymphocyte antigen 6 complex, locus E |
| 121 | GenBank | Hs.332040 | fulllength | 84773 | NM_032687; hypothetical protein MGC13010 |
| 122 | GenBank | Hs.12185 | fulllength | 84988 | NM_032902; protein phosphatase 1, regulatory (inhibitor) subunit 16A |
| 123 | GenBank | Hs.318725 | fulllength | 51105 | NM_016018; CGI-72 protein |
| 124 | GenBank | Hs.44159 | fulllength | 84165 | NM_032205; hypothetical protein FLJ21615 |
| 125 | GenBank | Hs.318725 | fulllength | 51105 | NM_016018; CGI-72 protein |
| 126 | GenBank | Hs.15611 | | 57623 | |
| 127 | GenBank | Hs.10669 | fulllength | 50807 | |
| 128 | GenBank | Hs.10669 | fulllength | 50807 | |
| 129 | GenBank | Hs.344478 | fulllength | 286053 | NM_173685; hypothetical protein FLJ32440 |
| 130 | GenBank | Hs.100691 | | | |
| 131 | GenBank | Hs.95631 | fulllength | 93594 | NM_145647; unknown MGC21654 product |
| 132 | GenBank | Hs.241576 | fulllength | 55493 | |
| 133 | GenBank | Hs.49136 | | | |
| 134 | GenBank | Hs.222088 | fulllength | 29028 | NM_014109; PRO2000 protein NM_032365; |
| 135 | RefSeq | Hs.30209 | fulllength | 22882 | NM_014943; transcription factor ZHX2 |
| 136 | RefSeq | Hs.79070 | fulllength | 4609 | NM_002467; v-myc myelocytomatosis viral oncogene homolog |
| 137 | RefSeq | Hs.95011 | fulllength | 6641 | NM_021021; basic beta 1 syntrophin |
| 138 | GenBank | Hs.432544 | | | |
| 139 | RefSeq | Hs.283740 | fulllength | 56943 | NM_020189; DC6 protein |
| 140 | GenBank | Hs.9222 | fulllength | 9166 | NM_004215; estrogen receptor binding site associated antigen 9 |
| 141 | RefSeq | Hs.169111 | fulllength | 55074 | NM_018002; oxidation resistance 1 |
| 142 | RefSeq | Hs.211458 | fulllength | 81501 | NM_030788; dendritic cell-specific transmembrane protein |
| 143 | GenBank | Hs.169111 | fulllength | 55074 | NM_018002; oxidation resistance 1 |
| 144 | GenBank | Hs.169111 | fulllength | 55074 | NM_018002; oxidation resistance 1 |
| 145 | GenBank | Hs.106673 | fulllength | 3646 | NM_001568; murine mammary tumor integration site 6 (oncogene |
| | | | | | homolog) |
| 146 | GenBank | Hs.290880 | | | |
| 147 | RefSeq | Hs.351475 | fulllength | 5440 | NM_005034; DNA directed RNA polymerase II polypeptide K |
| 148 | RefSeq | Hs.86905 | fulllength | 528 | NM_001695; ATPase, H+ transporting, lysosomal 42 kD, V1 subunit C, |
| | | | | | isoform 1 |
| 149 | GenBank | Hs.273344 | fulllength | 25879 | NM_014156; NM_015420; DKFZP564O0463 protein |
| 150 | RefSeq | Hs.197335 | fulllength | 10404 | NM_006102; NM_016134; plasma glutamate carboxypeptidase |
| 151 | GenBank | Hs.16621 | fulllength | 25962 | NM_015496; DKFZP43AI116 protein |
| 152 | RefSeq | Hs.279521 | fulllength | 55656 | NM_017864; hypothetical protein FLJ20530 |
| 153 | GenBank | Hs.6390 | fulllength | | |
| 154 | GenBank | Hs.6390 | fulllength | | |
| 155 | GenBank | Hs.243901 | fulllength | | |
| 156 | GenBank | Hs.29724 | fulllength | 79666 | NM_024613; phafin 2 |
| 157 | GenBank | Hs.50273 | est | | |
| 158 | GenBank | Hs.6390 | fulllength | | |
| 159 | GenBank | Hs.165539 | | | |
| 160 | GenBank | Hs.18426 | fulllength | 10247 | NM_005836; translational inhibitor protein p14.5 |
| 161 | GenBank | Hs.131255 | fulllength | 7381 | NM_006294; ubiquinol-cytochrome c reductase binding protein |
| 162 | GenBank | Hs.48320 | fulllength | 25897 | NM_015435; ring finger protein 19 |
| 163 | GenBank | Hs.14158 | fulllength | 8895 | NM_003909; copine III |
| 164 | GenBank | Hs.180612 | fulllength | 5828 | NM_000318; peroxisomal membrane protein 3 |
| 165 | GenBank | Hs.25812 | fulllength | 4683 | NM_002485; nibrin |
| 166 | RefSeq | Hs.25812 | fulllength | 4683 | NM_002485; nibrin |
| 167 | RefSeq | Hs.90800 | fulllength | 4325 | NM_005941; matrix metalloproteinase 16 isoform 1 NM_022564; matrix |
| | | | | | metalloproteinase 16 isoform 2 |
| 168 | GenBank | Hs.25812 | fulllength | 4683 | NM_002485; nibrin |
| 169 | GenBank | Hs.25812 | fulllength | 4683 | NM_002485; nibrin |
| 170 | RefSeq | Hs.96870 | fulllength | 27067 | NM_014393; staufen homolog 2 |
| 171 | RefSeq | Hs.106650 | fulllength | 54968 | NM_017866; hypothetical protein FLJ20533 |
| 172 | GenBank | Hs.106650 | fulllength | 54968 | NM_017866; hypothetical protein FLJ20533 |
| 173 | GenBank | Hs.168950 | | | |
| 174 | RefSeq | Hs.4147 | fulllength | 23471 | NM_014294; translocating chain-associating membrane protein |
| 175 | RefSeq | Hs.94631 | fulllength | 10565 | NM_006421; brefeldin A-inhibited guanine nucleotide-exchange protein 1 |
| 176 | GenBank | Hs.94631 | fulllength | 10565 | NM_006421; brefeldin A-inhibited guanine nucleotide-exchange protein 1 |
| 177 | GenBank | Hs.406325 | fulllength | 5150 | NM_002603; phosphodiesterase 7A isoform a NM_002604; |
| | | | | | phosphodiesterase 7A isoform b |
| 178 | GenBank | Hs.97603 | | | |
| 179 | GenBank | Hs.306621 | | | |
| 180 | RefSeq | Hs.380877 | fulllength | 23678 | NM_013257; serum/glucocorticoid regulated kinase-like isoform 1 |
| | | | | | NM_170709; serum/glucocorticoid regulated kinase-like isoform 2 |
| 181 | GenBank | Hs.380877 | fulllength | 23678 | NM_013257; serum/glucocorticoid regulated kinase-like isoform 1 |
| | | | | | NM_170709; serum/glucocorticoid regulated kinase-like isoform 2 |
| 182 | RefSeq | Hs.106768 | fulllength | 55156 | NM_018120; armadillo repeat-containing protein |
| 183 | GenBank | Hs.106768 | fulllength | 55156 | NM_018120; armadillo repeat-containing protein |
| 184 | GenBank | Hs.332422 | fulllength | 157807 | NM_173519; hypothetical protein MGC34646 |
| 185 | GenBank | Hs.283664 | fulllength | 444 | NM_004318; aspartate beta-hydroxylase isoform a NM_020164; |
| | | | | | aspartate beta-hydroxylase isoform e NM_032466; aspartate beta- |
| | | | | | hydroxylase isoform c NM_032467; aspartate beta-hydroxylase isoform d |
| | | | | | NM_032468; aspartate beta-hydroxylase isoform b |
| 186 | GenBank | Hs.78305 | fulllength | 5862 | NM_002865; RAB2, member RAS oncogene family |
| 187 | GenBank | Hs.78305 | fulllength | 5862 | NM_002865; RAB2, member RAS oncogene family |
| 188 | GenBank | Hs.323164 | fulllength | 79145 | NM_024300; hypothetical protein MGC2217 |
| 189 | GenBank | Hs.60293 | fulllength | 115294 | NM_052937; similar to hypothetical protein FLJ10883 |
| 190 | GenBank | Hs.60293 | fulllength | 115294 | NM_052937; similar to hypothetical protein FLJ10883 |
| 191 | GenBank | Hs.79300 | fulllength | 7336 | NM_003350; ubiquitin-conjugating enzyme E2 variant 2 |
| 192 | RefSeq | Hs.72887 | fulllength | 1670 | NM_021010; defensin, alpha 5, preproprotein |
| 193 | RefSeq | Hs.26458 | fulllength | 4482 | NM_012331; methionine sulfoxide reductase A |
| 194 | GenBank | Hs.24724 | | | |
| 195 | GenBank | Hs.24724 | | | |
| 196 | GenBank | Hs.19575 | fulllength | 51606 | NM_015941; ATPase, H+ transporting, lysosomal 50/57 kDa, V1 subunit H |
| 197 | RefSeq | Hs.173381 | fulllength | 1808 | NM_001386; dihydropyrimidinase-like 2 |
| 198 | RefSeq | Hs.297939 | fulllength | 1508 | NM_001908; cathepsin B preproprotein NM_147780; cathepsin B |
| | | | | | preproprotein NM_147781; cathepsin B preproprotein NM_147782; |
| | | | | | cathepsin B preproprotein NM_147783; cathepsin B preproprotein |
| 199 | GenBank | Hs.352416 | fulllength | 57805 | NM_021174; p30 DBC protein |
| 200 | GenBank | Hs.8700 | fulllength | 10395 | NM_006094; deleted in liver cancer 1 NM_024767; deleted in liver |
| | | | | | cancer 1 |
| 201 | GenBank | Hs.8700 | fulllength | 10395 | NM_006094; deleted in liver cancer 1 NM_024767; deleted in liver |
| | | | | | cancer 1 |
| 202 | GenBank | Hs.5943 | fulllength | 51201 | NM_016353; rec |
| 203 | GenBank | Hs.5943 | fulllength | 51201 | NM_016353; rec |
| 204 | GenBank | Hs.300861 | | | |
| 205 | GenBank | Hs.89868 | fulllength | 23516 | |
| 206 | RefSeq | Hs.274150 | fulllength | 55124 | NM_018068; piwi-like 2 |
| 207 | RefSeq | Hs.267905 | fulllength | 55140 | NM_018091; elongation protein 3 homolog |
| 208 | GenBank | Hs.193974 | fulllength | 2936 | NM_000637; glutathione reductase |
| 209 | GenBank | Hs.137732 | fulllength | 23087 | NM_015066; tripartite motif-containing 35 isoform 1 NM_171982; |
| | | | | | tripartite motif-containing 35 isoform 2 |
| 210 | GenBank | Hs.99480 | | 157570 | |
| 211 | RefSeq | Hs.274136 | fulllength | 55290 | NM_018310; RNA polymerase III transcription initiation factor BRF2 |
| 212 | GenBank | Hs.194726 | fulllength | 9530 | NM_004874; BCL2-associated athanogene 4 |
| 213 | GenBank | Hs.406670 | fulllength | 84513 | NM_032483; HTPAP protein |
| 214 | GenBank | Hs.406670 | fulllength | 84513 | NM_032483; HTPAP protein |
| 215 | RefSeq | Hs.170822 | fulllength | 81790 | NM_030954; hypothetical protein DKFZp564A022 |
| 216 | GenBank | Hs.355753 | | 137964 | NM_178819; putative lysophosphatidic acid acyltransferase |
| 217 | GenBank | Hs.170822 | fulllength | 81790 | NM_030954; hypothetical protein DKFZp564A022 |
| 218 | GenBank | Hs.125849 | fulllength | 11160 | NM_007175; chromosome 8 open reading frame 2 |
| 219 | GenBank | Hs.125849 | fulllength | 11160 | NM_007175; chromosome 8 open reading frame 2 |
| 220 | GenBank | Hs.226573 | fulllength | 3551 | |
| 221 | GenBank | Hs.301959 | fulllength | 11212 | NM_007198; proline synthetase co-transcribed homolog |
| 222 | RefSeq | Hs.96094 | fulllength | 1142 | NM_000749; cholinergic receptor, nicotinic, beta polypeptide 3 |
| 223 | GenBank | Hs.226573 | fulllength | 3551 | |
| 224 | GenBank | Hs.34054 | fulllength | 90362 | NM_147189; hypothetical protein MGC39325 |
| 225 | GenBank | Hs.32352 | fulllength | 54775 | NM_017606; hypothetical protein DKFZp434K1210 |
| 226 | GenBank | Hs.293836 | | | |
| 227 | GenBank | Hs.90800 | fulllength | 4325 | NM_005941; matrix metalloproteinase 16 isoform 1 NM_022564; matrix |
| | | | | | metalloproteinase 16 isoform 2 |
| 228 | RefSeq | Hs.128856 | fulllength | 51435 | NM_016240; CSR1 protein |

TABLE 25
_inv(16)

| # | affy Id | HUGO name | F | p | q | Title | MapLocation | Sequence Type | Transcript ID |
|---|---|---|---|---|---|---|---|---|---|
| 1 | 218018_at | C21orf97 | 157.77 | 2.09e−16 | 8.17e−12 | chromosome 21 open reading frame 97 | 21q22.3 | Consensussequence | Hs.4746.0 |
| 2 | 212608_s_at | | 140.44 | 7.66e−15 | 1.50e−10 | Homo sapiens clone 23872 mRNA sequence | | Consensussequence | Hs.323470.0 |
| 3 | 228848_at | ABTB1 | 136.11 | 2.19e−14 | 2.85e−10 | ankyrin repeat and BTB (POZ) domain containing 1 | 3q21 | Consensussequence | Hs.107812.1 |
| 4 | 221357_at | CHRM4 | 132.03 | 3.41e−14 | 3.33e−10 | cholinergic receptor, muscarinic 4 | 11p12-p11.2 | Exemplarsequence | Hs.248100.0 |
| 5 | 210690_at | KLRC4 | 117.63 | 5.89e−05 | 3.37e−03 | killer cell lectin-like receptor subfamily C, member 4 | 12p13.2-p12.3 | Exemplarsequence | Hs.268510.0 |
| 6 | 205495_s_at | GNLY | 113.09 | 5.07e−14 | 3.96e−10 | granulysin | 2p12-q11 | Exemplarsequence | Hs.105806.1 |
| 7 | 218786_at | | 103.51 | 2.65e−08 | 1.23e−05 | ESTs | | Exemplarsequence | Hs.6341.0 |
| 8 | 234699_at | RNASE7 | 100.22 | 3.85e−13 | 2.34e−09 | ribonuclease, RNase A family, 7 | 14q11.1 | Consensussequence | Hs.307078.0 |
| 9 | 201454_s_at | NPEPPS | 99.82 | 6.90e−10 | 7.28e−07 | aminopeptidase puromycin sensitive | 17q21 | Consensussequence | Hs.293007.0 |
| 10 | 231558_at | INSM1 | 99.36 | 4.79e−13 | 2.34e−09 | insulinoma-associated 1 | 20p11.2 | Consensussequence | Hs.89584.1 |
| 11 | 202174_s_at | PCM1 | 98.55 | 3.22e−05 | 2.17e−03 | pericentriolar material 1 | 8p22-p21.3 | Exemplarsequence | Hs.75737.0 |
| 12 | 230881_at | FLJ32734 | 98.26 | 4.34e−13 | 2.34e−09 | hypothetical protein FLJ32734 | 17p13.1 | Consensussequence | Hs.121438.0 |
| 13 | 224755_at | SMBP | 97.89 | 4.72e−07 | 9.36e−05 | SM-11044 binding protein | 10q23.33 | Consensussequence | Hs.8203.1 |
| 14 | 241623_at | PTPN2 | 92.45 | 3.51e−09 | 2.48e−06 | protein tyrosine phosphatase, non-receptor type 2 | 18p11.3-p11.2 | Consensussequence | Hs.150755.0 |
| 15 | 211793_s_at | ABI-2 | 92.45 | 3.80e−10 | 5.11e−07 | abl-interactor 2 | 2q33 | Exemplarsequence | Hs.256315.3 |
| 16 | 200779_at | ATF4 | 91.63 | 8.12e−10 | 8.13e−07 | activating transcription factor 4 (tax-responsive enhancer element B67) | 22q13.1 | Exemplarsequence | Hs.181243.0 |
| 17 | 244777_at | | 89.48 | 1.41e−10 | 2.63e−07 | Homo sapiens, Similar to RIKEN cDNA 5730537H01 gene, clone | | Consensussequence | Hs.111418.0 |
| | | | | | | IMAGE: 4617463, mRNA | | | |
| 18 | 209996_x_at | PCM1 | 88.78 | 1.99e−09 | 1.65e−06 | pericentriolar material 1 | 8p22-p21.3 | Consensussequence | Hs.315478.0 |
| 19 | 221749_at | FLJ31657 | 87.59 | 6.72e−08 | 2.39e−05 | hypothetical protein FLJ31657 | 8q12.1 | Consensussequence | Hs.5518.0 |
| 20 | 206179_s_at | p25 | 86.81 | 4.14e−12 | 1.80e−08 | brain-specific protein p25 alpha | 5p15.3 | Exemplarsequence | Hs.29353.0 |
| 21 | 215693_x_at | DDX27 | 83.97 | 9.81e−07 | 1.63e−04 | DEAD/H (Asp-Glu-Ala-Asp/His) box polypeptide 27 | 20q13.13 | Consensussequence | Hs.65234.2 |
| 22 | 209510_at | TRC8 | 83.43 | 2.57e−09 | 2.05e−06 | patched related protein translocated in renal cancer | 8q24 | Exemplarsequence | Hs.28285.0 |
| 23 | 231179_at | IHPK3 | 82.29 | 1.13e−11 | 3.68e−08 | inositol hexaphosphate kinase 3 | 6p21.31 | Consensussequence | Hs.17253.0 |
| 24 | 204530_s_at | TOX | 80.94 | 3.28e−04 | 1.22e−02 | thymus high mobility group box protein TOX | 8q11.23 | Exemplarsequence | Hs.184297.0 |
| 25 | 208617_s_at | PTP4A2 | 80.90 | 1.02e−11 | 3.68e−08 | protein tyrosine phosphatase type IVA, member 2 | 1p35 | Exemplarsequence | Hs.82911.0 |
| 26 | 236296_x_at | FLJ34715 | 80.69 | 2.61e−05 | 1.87e−03 | hypothetical protein FLJ34715 | 8p21.2 | Consensussequence | Hs.197445.0 |
| 27 | 232532_at | DKFZP434P0316 | 80.42 | 4.69e−11 | 1.15e−07 | hypothetical protein DKFZp434P0316 | 17q25.3 | Consensussequence | Hs.252739.0 |
| 28 | 234480_at | DKFZP781C1711 | 80.10 | 1.11e−11 | 3.68e−08 | hypothetical protein DKFZp761C1711 | | Consensussequence | Hs.285786.0 |
| 29 | 229819_at | A1BG | 80.05 | 1.06e−07 | 3.21e−05 | alpha-1-B glycoprotein | 19q13.4 | Consensussequence | Hs.41997.0 |
| 30 | 233108_at | DSCR3 | 78.85 | 1.31e−11 | 3.93e−08 | Down syndrome critical region gene 3 | 21q22.2 | Consensussequence | Hs.296548.0 |
| 31 | 237314_at | MGC26778 | 78.50 | 5.13e−06 | 5.52e−04 | hypothetical protein MGC26778 | 10p12.1 | Consensussequence | Hs.232407.0 |
| 32 | 218150_at | ARL5 | 77.10 | 4.96e−10 | 5.77e−07 | ADP-ribosylation factor-like 5 | 2q23.3 | Exemplarsequence | Hs.42500.0 |
| 33 | 223921_s_at | GBA2 | 75.40 | 2.50e−11 | 6.91e−08 | glucosidase, beta (bile acid) 2 | 9p13.1 | Exemplarsequence | Hs.173422.0 |
| 34 | 214230_at | CDC42 | 74.64 | 5.35e−05 | 3.18e−03 | cell division cycle 42 (GTP binding protein, 25 kDa) | 1p36.1 | Consensussequence | Hs.146409.3 |
| 35 | 210022_at | NSPC1 | 73.75 | 4.43e−11 | 1.15e−07 | likely ortholog of mouse nervous system polycomb 1 | 2p12 | Exemplarsequence | Hs.316750.0 |
| 36 | 213545_x_at | SNX3 | 73.07 | 5.23e−10 | 5.83e−07 | sorting nexin 3 | 6q21 | Consensussequence | Hs.12102.2 |
| 37 | 242568_s_at | | 72.71 | 5.37e−11 | 1.23e−07 | Homo sapiens cDNA FLJ38922 fis, clone NT2NE2011691. | | Consensussequence | Hs.97259.1 |
| 38 | 207924_x_at | PAX8 | 71.11 | 6.51e−11 | 1.41e−07 | paired box gene 8 | 2q12-14 | Exemplarsequence | Hs.73149.2 |
| 39 | 218121_at | HMOX2 | 70.74 | 3.68e−08 | 1.55e−05 | heme oxygenase (decycling) 2 | 16p13.3 | Exemplarsequence | Hs.284279.0 |
| 40 | 243915_at | | 70.48 | 1.08e−10 | 2.22e−07 | ESTs, Weakly similar to hypothetical protein FLJ20378 [Homo sapiens] | | Consensussequence | Hs.205853.0 |
| | | | | | | [H. sapiens] | | | |
| 41 | 228786_at | | 69.79 | 4.52e−05 | 2.79e−03 | Homo sapiens cDNA FLJ31518 fis, clone NT2RI2000064. | | Consensussequence | Hs.55962.0 |
| 42 | 217429_at | | 68.96 | 9.73e−10 | 9.50e−07 | Homo sapiens mRNA; cDNA DKFZp564A216 (from clone | | Consensussequence | Hs.274505.0 |
| | | | | | | DKFZp564A216) | | | |
| 43 | 229617_x_at | FLJ22688 | 68.05 | 1.46e−08 | 7.50e−06 | hypothetical protein FLJ22688 | 19q13.33 | Consensussequence | Hs.288800.2 |
| 44 | 224741_x_at | | 67.99 | 1.27e−10 | 2.49e−07 | Homo sapiens mRNA; cDNA DKFZp564D0164 (from clone | | Consensussequence | Hs.289721.1 |
| | | | | | | DKFZp564D0164) | | | |
| 45 | 244271_at | | 67.08 | 2.07e−10 | 3.11e−07 | ESTs, Weakly similar 10 hypothetical protein FLJ20378 [Homo sapiens] | | Consensussequence | Hs.205015.0 |
| | | | | | | [H. sapiens] | | | |
| 46 | 232519_at | | 66.99 | 1.76e−10 | 2.87e−07 | Homo sapiens cDNA FLJ13523 fis, clone PLACE1005968. | | Consensussequence | Hs.12865.1 |
| 47 | 202344_at | HSF1 | 66.73 | 1.70e−10 | 2.87e−07 | heat shock transcription factor 1 | 8q24.3 | Exemplarsequence | Hs.1499.0 |
| 48 | 203420_at | FAM8A1 | 65.92 | 1.87e−10 | 2.93e−07 | family with sequence similarity 8, member A1 | 6p22-p23 | Exemplarsequence | Hs.95260.0 |
| 49 | 228902_at | NUP214 | 65.83 | 1.73e−10 | 2.87e−07 | nucleoporin 214 kDa | 9q34.1 | Consensussequence | Hs.170285.2 |
| 50 | 242652_at | | 65.79 | 8.76e−08 | 2.79e−05 | ESTs | | Consensussequence | Hs.191754.0 |

| # | Sequence Derived From | Sequence ID | Sequence Source | Unigene_Accession | Cluster_Type | LocusLink | Full_Length_Reference_Seq |
|---|---|---|---|---|---|---|---|
| 1 | NM_021941.1 | Hs.4746.0 | GenBank | Hs.4746 | fulllength | 60683 | NM_021941; chromosome 21 open reading frame 97 |
| 2 | W85912 | Hs.323470.0.A1 | GenBank | Hs.188882 | | | |
| 3 | AW511257 | Hs.107812.1.A1 | GenBank | Hs.107812 | fulllength | 80325 | NM_032548; ankyrin repeat and BTB (POZ) domain containing 1 isoform 1 |
| | | | | | | | NM_172027; ankyrin repeat and BTB (POZ) domain containing 1 isoform 2 |
| | | | | | | | NM_172028; ankyrin repeat and BTB (POZ) domain containing 1 isoform 3 |
| 4 | NM_000741.1 | g4502820 | RefSeq | Hs.248100 | fulllength | 1132 | NM_000741; cholinergic receptor, muscarinic 4 |
| 5 | U96845.1 | g2673988 | GenBank | Hs.268510 | fulllength | 8302 | NM_013431; killer cell lectin-like receptor subfamily C, member 4 |
| 6 | NM_006433.2 | g7108343 | RefSeq | Hs.105806 | fulllength | 10578 | NM_006433; granulysin isoform NKG5 NM_012483; granulysin isoform 519 |
| 7 | NM_016575.1 | g7706748 | RefSeq | Hs.374350 | est | | |
| 8 | AJ131212.1 | Hs.307078.0 | GenBank | Hs.307078 | fulllength | 84659 | NM_032572; ribonuclease 7 |
| 9 | NM_006310.1 | Hs.293007.0_RC | GenBank | Hs.293007 | fulllength | 9520 | NM_006310; aminopeptidase puromycin sensitive |
| 10 | BF108585 | Hs.89584.1_RC | GenBank | Hs.89584 | fulllength | 3642 | NM_002196; insulinoma-associated 1 |
| 11 | NM_006197.1 | g5453855 | RefSeq | Hs.75737 | fulllength | 5108 | NM_006197; pericentriolar material 1 |
| 12 | AI200853 | Hs.121438.0.A1 | GenBank | Hs.121438 | fulllength | 146849 | NM_144681; hypothetical protein FLJ32734 |
| 13 | BE621524 | Hs.8203.1 | GenBank | Hs.8203 | fulllength | 56889 | NM_020123; endomembrane protein emp70 precursor isolog |
| 14 | AL119890 | Hs.150755.0 | GenBank | Hs.82829 | fulllength | 5771 | NM_002828; protein tyrosine phosphatase, non-receptor type 2 isoform 1 |
| | | | | | | | NM_080422; protein tyrosine phosphatase, non-receptor type 2 isoform 2 |
| | | | | | | | NM_080423; protein tyrosine phosphatase, non-receptor type 2 |
| 15 | AF260261.1 | g7839523 | GenBank | Hs.343575 | fulllength | 10152 | NM_005759; abl-interactor 2 |
| 16 | NM_001675.1 | g4502264 | RefSeq | Hs.181243 | fulllength | 468 | NM_001675; activating transcription factor 4 |
| 17 | AA504595 | Hs.111418.0.A1 | GenBank | Hs.367634 | | | |
| 18 | AA931266 | Hs.315478.0 | GenBank | Hs.75737 | fulllength | 5108 | NM_006197; pericentriolar material 1 |
| 19 | AU157915 | Hs.5518.0.S1 | GenBank | Hs.5518 | fulllength | 253943 | NM_152758; hypothetical protein FLJ31657 |
| 20 | NM_007030.1 | g5902017 | RefSeq | Hs.29353 | fulllength | 11076 | NM_007030; brain-specific protein p25 alpha |
| 21 | AL512707.1 | Hs.65234.2 | GenBank | Hs.65234 | fulllength | 55661 | NM_017895; DEAD/H (Asp-Glu-Ala-Asp/His) box polypeptide 27 |
| 22 | AF064801.1 | g3395786 | GenBank | Hs.28285 | fulllength | 11236 | NM_007218; ring finger protein 139 |
| 23 | R99291 | Hs.17253.0.A1 | GenBank | Hs.17253 | fulllength | 117283 | NM_054111; inositol hexaphosphate kinase 3 |
| 24 | NM_014729.1 | g7662321 | RefSeq | Hs.184297 | fulllength | 9760 | NM_014729; thymus high mobility group box protein TOX |
| 25 | AF208850.1 | g7582287 | GenBank | Hs.82911 | fulllength | 8073 | NM_003479; protein tyrosine phosphatase type IVA, member 2 isoform 1 |
| | | | | | | | NM_080391; protein tyrosine phosphatase type IVA, member 2 isoform 1 |
| | | | | | | | NM_080392; protein tyrosine phosphatase type IVA, member 2 isoform 2 |
| 26 | AW295176 | Hs.197445.0.A1 | GenBank | Hs.375560 | fulllength | 286057 | NM_173686; hypothetical protein FLJ34715 |
| 27 | AL136774.1 | Hs.252739.0 | GenBank | Hs.252739 | fulllength | 84074 | NM_032134; hypothetical protein DKFZp434P0316 |
| 28 | AL137340.1 | Hs.285786.0 | GenBank | Hs.285786 | | 57796 | |
| 29 | AI022193 | Hs.41997.0_RC | GenBank | Hs.373554 | fulllength | 1 | NM_130786; alpha 1B-glycoprotein |
| 30 | AW613396 | Hs.296548.0_RC | GenBank | Hs.26146 | fulllength | 10311 | NM_006052; Down syndrome critical region protein 3 |
| 31 | AW119023 | Hs.232407.0_RC | GenBank | Hs.209200 | fulllength | 219670 | NM_145010; hypothetical protein MGC26778 |
| 32 | NM_012097.1 | g6912243 | RefSeq | Hs.342849 | fulllength | 26225 | NM_012097; ADP-ribosylatlon factor-like 5 isoform 1 NM_177985; ADP- |
| | | | | | | | ribosylation factor-like 5 isoform 2 |
| 33 | AF258662.1 | g12005895 | GenBank | Hs.173422 | fulllength | 57704 | NM_020944; bile acid beta-glucosidase |
| 34 | R37664 | Hs.146409.3.S1 | GenBank | Hs.146409 | fulllength | 998 | NM_001791; cell division cycle 42 isoform 1 NM_044472; cell division cycle 42 |
| | | | | | | | isoform 2 |
| 35 | BC004952.1 | g13436325 | GenBank | Hs.316750 | fulllength | 84759 | NM_032673; hypothetical protein MGC10882 |
| 36 | BE962615 | Hs.12102.2_RC | GenBank | Hs.12102 | fulllength | 8724 | NM_003795; sorting nexin 3 isoform a NM_152827; sorting nexin 3 isoform b |
| | | | | | | | NM_152828; sorting nexin 3 isoform c |
| 37 | BF995452 | Hs.97259.1 | GenBank | Hs.435026 | | | |
| 38 | NM_013992.1 | g7669541 | RefSeq | Hs.73149 | fulllength | 7849 | NM_003466; paired box gene 8 isoform PAX8A NM_013951; paired box |
| | | | | | | | gene 8 isoform PAX8B NM_013952; paired box gene 8 isoform PAX8C |
| | | | | | | | NM_013953; paired box gene 8 isoform PAX8D NM_013992; paired box |
| | | | | | | | gene 8 isoform PAX8E |
| 39 | NM_002134.2 | g8051607 | RefSeq | Hs.284279 | fulllength | 3163 | NM_002134; heme oxygenase (decyclizing) 2 |
| 40 | AW130385 | Hs.205853.0 | GenBank | Hs.369571 | est | | |
| 41 | AA909523 | Hs.55962.0_RC | GenBank | Hs.433110 | | | |
| 42 | AL049307.1 | Hs.274505.0.A1 | GenBank | Hs.274505 | | | |
| 43 | AA729495 | Hs.288800.2.A1 | GenBank | Hs.288800 | fulllength | 80199 | NM_025129; hypothetical protein FLJ22688 |
| 44 | BG329175 | Hs.289721.1.S1 | GenBank | Hs.289721 | | | |
| 45 | AI697709 | Hs.205015.0_RC | GenBank | Hs.205015 | est | | |
| 46 | AK023585.1 | Hs.12865.1.A1 | GenBank | Hs.343962 | | | |
| 47 | NM_005526.1 | g5031766 | RefSeq | Hs.380935 | fulllength | 3297 | NM_005526; heat shock transcription factor 1 |
| 48 | NM_016255.1 | g7705267 | RefSeq | Hs.95260 | fulllength | 51439 | NM_016255; Autosomal Highly Conserved Protein |
| 49 | AA516455 | Hs.170285.2 | GenBank | Hs.170285 | fulllength | 8021 | NM_005085; nucleoporin 214 kDa isoform 2 NM_153642; nucleoporin |
| | | | | | | | 214 kDa isoform 1 |
| 50 | AI760942 | Hs.191754.0_RC | GenBank | Hs.445968 | est | | |

TABLE 26
_inv(16)

| | | | | | | | Sequence | |
|---|---|---|---|---|---|---|---|---|
| # | affy id | HUGO name | Title | MapLocation | Sequence Type | Transcript ID | Derived From | Sequence ID |
| 1 | 225676_s_at | DKFZP564O0463 | DKFZP564O0463 protein | 8q22.3 | Consensussequence | Hs.273344.1 | AK001693.1 | Hs.273344.1 |
| 2 | 208423_s_at | MSR1 | macrophage scavenger receptor 1 | 8p22 | Exemplarsequence | Hs.49.0 | NM_002445.1 | g4505258 |
| 3 | 218187_s_at | FLJ20989 | hypothetical protein FLJ20989 | | Exemplarsequence | Hs.169615.0 | NM_023080.1 | g12751496 |
| 4 | 202344_at | HSF1 | heat shock transcription factor 1 | 8q24.3 | Exemplarsequence | Hs.1499.0 | NM_005526.1 | g5031766 |
| 5 | 209899_s_at | SIAHBP1 | fuse-binding protein-interacting repressor | 8q24.2-qtel | Exemplarsequence | Hs.74562.0 | AF217197.1 | g6740005 |
| 6 | 33132_at | CPSF1 | cleavage and polyadenylation specific factor 1, 160 kDa | 8q24.23 | Consensussequence | 8 | U37012 | 4923232 |
| 7 | 229802_at | | Homo sapiens cDNA FLJ14388 fis, clone HEMBA1002716. | | Consensussequence | Hs.9812.0 | AA147884 | Hs.9812.0.A1 |
| 8 | 213122_at | KIAA1750 | KIAA1750 protein | 8q22.1 | Consensussequence | Hs.173094.0 | AI096375 | Hs.173094.0.S1 |
| 9 | 204865_at | CA3 | carbonic anhydrase III, muscle specific | 8q13-q22 | Exemplarsequence | Hs.82129.0 | NM_005181.2 | g6996001 |
| 10 | 201398_s_at | TRAM | translocating chain-associating membrane protein | 8q13.1 | Exemplarsequence | Hs.4147.0 | BC000687.1 | g12653796 |
| 11 | 203269_at | NSMAF | neutral sphingomyelinase (N-SMase) activation associated factor | 8q12-q13 | Exemplarsequence | Hs.78687.0 | NM_003580.1 | g4505464 |
| 12 | 226483_at | FLJ32370 | hypothetical protein FLJ32370 | 8q11.23 | Consensussequence | Hs.280858.1 | AI890761 | Hs.280858.1.A1 |
| 13 | 221367_at | MOS | v-mos Moloney murine sarcoma viral oncogene homolog | 8q11 | Exemplarsequence | Hs.248146.0 | NM_005372.1 | g4885488 |
| 14 | 233101_at | | Homo sapiens cDNA FLJ12009 fis, clone HEMBB1001618. | | Consensussequence | Hs.287478.0 | AK022071.1 | Hs.287478.0 |
| 15 | 242387_at | INM01 | hypothetical protein INM01 | 8p23.3 | Consensussequence | Hs.289293.0 | BF509686 | Hs.289293.0 |
| 16 | 208647_at | FDFT1 | farnesyl-diphosphate farnesyltransferase 1 | 8p23.1-p22 | Consensussequence | Hs.48876.1 | AA872727 | Hs.48876.1 |
| 17 | 218096_at | FLJ11210 | hypothetical protein FLJ11210 | 8p23.1 | Exemplarsequence | Hs.27842.0 | NM_018361.1 | g8922941 |
| 18 | 200090_at- | FNTA | farnesyltransferase, CAAX box, alpha | 8p22-q11 | Consensussequence | Hs.138381.1 | BG168896 | Hs.138381.1.A1 |
| | HG-U133A | | | | | | | |
| 19 | 218250_s_at | CNOT7 | CCR4-NOT transcription complex, subunit 7 | 8p22-p21.3 | Exemplarsequence | Hs.226318.0 | NM_013354.2 | g10518495 |
| 20 | 202174_s_at | PCM1 | pericentriolar material 1 | 8p22-p21.3 | Exemplarsequence | Hs.75737.0 | NM_006197.1 | g5453855 |
| 21 | 214118_x_at | PCM1 | pericentriolar material 1 | 8p22-p21.3 | Consensussequence | Hs.75737.1 | AI205598 | Hs.75737.1.S1 |
| 22 | 214937_x_at | PCM1 | pericentriolar material 1 | 8p22-p21.3 | Consensussequence | Hs.75737.2 | AI924817 | Hs.75737.2.S1 |
| 23 | 214054_at | DOK2 | docking protein 2, 56 kDa | 8p21.2 | Consensussequence | Hs.71215.0 | AI828929 | Hs.71215.0 |
| 24 | 207595_s_at | BMP1 | bone morphogenetic protein 1 | 8p21 | Exemplarsequence | Hs.1274.3 | NM_006132.1 | g5902814 |
| 25 | 222544_s_at | WHSC1L1 | Wolf-Hirschhom syndrome candidate 1-like 1 | 8p11.2 | Consensussequence | Hs.27721.0 | AI697751 | Hs.27721.0_RC |
| 26 | 230361_at | | ESTs, Weakiy similar to inner centromere protein [Mus musculus] | | Consensussequence | Hs.146109.0 | AW664013 | Hs.146109.0_RC |
| | | | [M. musculus] | | | | | |
| 27 | 219606_at | CGI-72 | CGI-72 protein | 8q24.3 | Exemplarsequence | Hs.318725.0 | NM_016018.1 | g7705782 |
| 28 | 225488_at | MGC3067 | hypothetical protein MGC3067 | 8q24.13 | Consensussequence | Hs.323114.1 | AI967978 | Hs.323114.1 |
| 29 | 205608_s_at | ANGPT1 | angiopoietin 1 | 8q22.3-q23 | Exemplarsequence | Hs.2463.0 | U83508.1 | g1907326 |
| 30 | 205609_at | ANGPT1 | angiopoietin 1 | 8q22.3-q23 | Exemplarsequence | Hs.2463.0 | NM_001146.1 | g4502086 |
| 31 | 220843_s_at | DKFZP564O0463 | DKFZP564O0463 protein | 8q22.3 | Exemplarsequence | Hs.273344.0 | NM_014156.1 | g7661767 |
| 32 | 235509_at | MGC40214 | hypothetical protein MGC40214 | 8q22.1 | Consensussequence | Hs.98471.0 | AV662196 | Hs.98471.0_RC |
| 33 | 231268_at | | Homo sapiens, clone IMAGE: 5222754, mRNA, partial cds | | Consensussequence | Hs.144027.0 | AI539459 | Hs.144027.0.A1 |
| 34 | 213906_at | MYBL1 | v-myb myeloblastosis viral oncogene homolog (avian)-like 1 | 8q22 | Consensussequence | Hs.300592.0 | AW592266 | Hs.300592.0.S1 |
| 35 | 219819_s_at | MRPS28 | mitochondrial ribosomal protein S28 | 8q21.1-q21.2 | Exemplarsequence | Hs.55097.0 | NM_014018.1 | g7661729 |
| 36 | 203208_s_at | CHPPR | likely ortholog of chicken chondrocyte protein with a poly-proline region | 8q12.1 | Exemplarsequence | Hs.170198.0 | NM_014637.1 | g7661853 |
| 37 | 223907_s_at | PINX1 | PIN2-interacting protein 1 | 8p23 | Exemplarsequence | Hs.99829.0 | AF205718.1 | g10504237 |
| 38 | 235801_at | N33 | Putative prostate cancer tumor suppressor | 8p22 | Consensussequence | Hs.283526.0 | AI760262 | Hs.283526.0_RC |
| 39 | 230977_at | LOC286056 | hypothetical protein LOC286056 | 8p21.2 | Consensussequence | Hs.131055.0 | AI016313 | Hs.131055.0.A1 |
| 40 | 207287_at | FLJ14107 | hypothetical protein FLJ14107 | 8p21.2 | Exemplarsequence | Hs.287624.0 | NM_025026.1 | g13376547 |
| 41 | 210029_at | INDO | indoleamine-pyrrole 2,3 dioxygenase | 8p12-p11 | Exemplarsequence | Hs.840.0 | M34455.1 | g185790 |
| 42 | 232040_at | | Homo sapiens cDNA: FLJ22090 fis, clone | | Consensussequence | Hs.170296.0 | AK025743.1 | Hs.170296.0 |
| | | | HEP16084. | | | | | |
| 43 | 203534_at | LSM1 | LSM1 homolog, U6 small nuclear RNA | 8p11.2 | Exemplarsequence | Hs.111783.0 | NM_014462.1 | g7657312 |
| | | | associated (S. cerevisiae) | | | | | |
| 44 | 208820_at | PTK2 | PTK2 protein tyrosine kinase 2 | 8q24-qter | Consensussequence | Hs.740.1 | AL037339 | Hs.740.1.S2 |
| 45 | 201618_x_at | GPAA1 | GPAA1P anchor attachment protein 1 | 8q24.3 | Exemplarsequence | Hs.4742.0 | NM_003801.2 | g6031166 |
| | | | homolog (yeast) | | | | | |
| 46 | 200936_at | RPL8 | ribosomal protein L8 | 8q24.3 | Exemplarsequence | Hs.178551.0 | NM_000973.1 | g4506662 |
| 47 | 213072_at | LOC157542 | hypothetical protein BC004544 | 8q24.3 | Consensussequence | Hs.331601.0 | AI928387 | Hs.331601.0 |
| 48 | 58696_at | FLJ20591 | exosome component Rrp41 | 8q24.3 | Consensussequence | 5 | AL039469 | 4923514_rc |
| 49 | 218151_x_at | FLJ11856 | putative G-protein coupled receptor | 8q24.3 | Exemplarsequence | Hs.6459.0 | NM_024531.1 | g13375681 |
| | | | GPCR41 | | | | | |
| 50 | 206574_s_at | PTP4A3 | protein tyrosine phosphatase type IVA, member 3 | | Exemplarsequence | Hs.43666.0 | NM_007079.1 | g6857821 |
| 51 | 227023_at | GLI4 | GLI-Kruppel family member GLI4 | 8q24.3 | Consensussequence | Hs.239451.0 | AI570458 | Hs.239451.0.A1 |
| 52 | 213756_s_at | HSF1 | heat shock transcription factor 1 | 8q24.3 | Consensussequence | Hs.1499.1 | AI393937 | Hs.1499.1 |
| 53 | 228405_at | RHPN1 | rhophilin, Rho GTPase binding protein 1 | 8q24.3 | Consensussequence | Hs.149152.0 | AI917311 | Hs.149152.0.A1 |
| 54 | 236533_at | DDEF1 | development and differentiation enhancing | 8q24.1-q24.2 | Consensussequence | Hs.199057.0 | AW236958 | Hs.199057.0.A1 |
| | | | factor 1 | | | | | |
| 55 | 219060_at | FLJ10204 | hypothetical protein FLJ10204 | 8q24.13 | Exemplarsequence | Hs.18029.0 | NM_018024.1 | g8922280 |
| 56 | 219402_s_at | MGC3067 | hypothetical protein MGC3067 | 8q24.13 | Exemplarsequence | Hs.323114.0 | NM_024295.1 | g13236515 |
| 57 | 225801_at | | Homo sapiens cDNA FLJ13329 fis, clone | | Consensussequence | Hs.61661.0 | BE877195 | Hs.61661.0_RC |
| | | | OVARC1001795. | | | | | |
| 58 | 222740_at | PRO2000 | PRO2000 protein | 8q24.13 | Consensussequence | Hs.46677.0 | AI925583 | Hs.46677.0_RC |
| 59 | 238562_at | | ESTs, Weakly similar to hypothetical | | | | | |
| | | | protein FLJ20378 [Homo sapiens] | | Consensussequence | Hs.280297.0 | BE542779 | Hs.280297.0.A1 |
| | | | [H. sapiens] | | | | | |
| 60 | 201592_at | EIF3S3 | eukaryotic translation initiation factor 3, subunit 3 | 8q23.3 | Exemplarsequence | Hs.58189.0 | NM_003756.1 | g4503514 |
| | | | gamma, 40 kDa | | | | | |
| 61 | 227786_at | PFDN2 | prefoldin 2 | 1q23.1 | Consensussequence | Hs.298229.2 | AI026938 | Hs.298229.2.A1 |
| 62 | 209510_at | TRC8 | patched related protein translocated in | 8q24 | Exemplarsequence | Hs.28285.0 | AF064801.1 | g3395786 |
| | | | renal cancer | | | | | |
| 63 | 219548_at | ZNF16 | zinc finger protein 16 (KOX 9) | 8q24 | Exemplarsequence | Hs.23019.0 | NM_006958.1 | 911177859 |
| 64 | 204932_at | TNFRSF11B | tumor necrosis factor receptor superfamily, | 8q24 | Consensussequence | Hs.81791.0 | BF433902 | Hs.81791.0 |
| | | | member 11b (osteoprotegerin) | | | | | |
| 65 | 240228_at | KIAA1894 | KIAA1894 protein | 8q23.3 | Consensussequence | Hs.21034.0 | AI187364 | Hs.21034.0_RC |
| 66 | 226776_at | DC6 | DC6 protein | 8q23.2 | Consensussequence | Hs.44243.0 | BF215862 | Hs.44243.0_RC |
| 67 | 208697_s_at | EIF3S6 | eukaryotic translation initiation factor 3, | 8q22-q23 | Exemplarsequence | Hs.106673.0 | BC000734.1 | g12653884 |
| | | | subunit 6 48 kDa | | | | | |
| 68 | 218899_s_at | BAALC | brain and acute leukemia, cytoplasmic | 8q22.3 | Exemplarsequence | Hs.169395.0 | NM_024812.1 | g13376199 |
| 69 | 202873_at | | Homo sapiens cDNA FLJ33383 fis, clone | | Consensussequence | Hs.86905.0 | NM_001695.1 | Hs.86905.0 |
| | | | BRACE2006514. | | | | | |
| 70 | 219388_at | FLJ13782 | hypothetical protein FLJ13782 | 8q22.3 | Exemplarsequence | Hs.257924.0 | NM_024915.1 | g13376381 |
| 71 | 203501_at | PGCP | plasma glutamate carboxypeptidase | 8q22.2 | Exemplarsequence | Hs.197335.0 | NM_006102.1 | g5174626 |
| 72 | 222950_at | FLJ13955 | hypothetical protein FLJ13955 | 8q22.1 | Consensussequence | Hs.127331.0 | NM_024759.1 | Hs.127331.0_RC |
| 73 | 212250_at | | Homo sapiens, Similar to LYRIC, clone | | Consensussequence | Hs.243901.0 | AI972475 | Hs.243901.0.S1 |
| | | | MGC: 41931 IMAGE: 5298467, mRNA, | | | | | |
| | | | complete cds | | | | | |
| 74 | 227277_at | | Homo sapiens cDNA FLJ40968 fis, clone | | Consensussequence | Hs.33074.0 | BG530089 | Hs.33074.0.S1 |
| | | | UTERU2012615. | | | | | |
| 75 | 235391_at | LOC137392 | similar to CG6405 gene product | 8q21.3 | Consensussequence | Hs.87672.0 | AW960748 | Hs.87672.0_RC |
| 76 | 238229_at | | ESTs | | Consensussequence | Hs.116240.0 | AI187388 | Hs.116240.0.A1 |
| 77 | 218273_s_at | PDP | pyruvate dehydrogenase phosphatase | 8q21.3 | Exemplarsequence | Hs.22265.0 | NM_018444.1 | g8923959 |
| 78 | 222572_at | PDP | pyruvate dehydrogenase phosphatase | 8q21.3 | Consensussequence | Hs.22265.0 | BG542521 | Hs.22265.0.S2 |
| 79 | 220549_at | FSBP | fibrinogen silencer binding protein | 8q22.1 | Exemplarsequence | Hs.241384.0 | NM_006550.1 | g5729829 |
| 80 | 219494_at | RAD54B | RAD54B homolog | 8q21.3-q22 | Exemplarsequence | Hs.128501.0 | NM_012415.1 | g6912621 |
| 81 | 207386_at | CYP7B1 | cytochrome P450, family 7, subfamily B, | 8q21.3 | Exemplarsequence | Hs.144877.0 | NM_004820.2 | g13787190 |
| | | | polypeptide 1 | | | | | |
| 82 | 202119_s_at | CPNE3 | copine III | 8q21.13 | Exemplarsequence | Hs.14158.0 | NM_003909.1 | g4503014 |
| 83 | 222051_s_at | E2F5 | E2F transcription factor 5, p130-binding | 8q21.13 | Consensussequence | Hs.2331.2 | AW139195 | Hs.2331.2 |
| 84 | 203011_at | IMPA1 | inositol(myo)-1(or 4)-monophosphatase 1 | 8q21.13-q21.3 | Exemplarsequence | Hs.171776.0 | NM_005536.2 | g8393607 |
| 85 | 218919_at | FLJ14007 | hypothetical protein FLJ14007 | 8q21.12 | Exemplarsequence | Hs.99519.0 | NM_024699.1 | g13375984 |
| 86 | 226803_at | MGC22825 | hypothetical protein MGC22825 | 8q21.12 | Consensussequence | Hs.183861.0 | AK000049.1 | Hs.183861.0.S1 |
| 87 | 209928_s_at | MSC | musculin (activated B-cell factor-1) | 8q21 | Exemplarsequence | Hs.42474.0 | AF060154.1 | g3089604 |
| 88 | 208361_s_at | BN51T | BN51 (BHK21) temperature sensitivity | 8q21 | Exemplarsequence | Hs.1276.0 | NM_001722.1 | g4502436 |
| | | | complementing | | | | | |
| 89 | 223475_at | LOC83690 | CocoaCrisp | 8q13.3 | Exemplarsequence | Hs.182364.0 | AF142573.1 | g12002310 |
| 90 | 230661_at | | Homo sapiens mRNA; cDNA DKFZp667P166 (from clone | | Consensussequence | Hs.194024.0 | AW451999 | Hs.194024.0_RC |
| | | | DKFZp667P166) | | | | | |
| 91 | 200717_x_at | RPL7 | ribosomal protein L7 | 8q13.2 | Exemplarsequence | Hs.153.0 | NM_000971.1 | g4506658 |
| 92 | 234768_at | | Homo sapiens cDNA: FLJ20865 fis, clone | | Consensussequence | Hs.306681.0 | AK024518.1 | Hs.306681.0 |
| | | | ADKA01850. | | | | | |
| 93 | 202955_s_at | BIG1 | brefeldin A-inhibited guanine nucleotide- | 8q13 | Exemplarsequence | Hs.94631.0 | AF084520.1 | g5052120 |
| | | | exchange protein 1 | | | | | |
| 94 | 203448_s_at | TERF1 | telomeric repeat binding factor (NIMA- | 8q13 | Consensussequence | Hs.194562.0 | AI347136 | Hs.194562.0.A1 |
| | | | interacting) 1 | | | | | |
| 95 | 219810_at | VCIP135 | valosin-containing protein (p97)/p47 | 8q13 | Exemplarsequence | Hs.287727.0 | NM_025054.1 | g13376584 |
| | | | complex-interacting protein p135 | | | | | |
| 96 | 221749_at | FLJ31657 | hypothetical protein FLJ31657 | 8q12.1 | Consensussequence | Hs.5518.0 | AU157915 | Hs.5518.0.S1 |
| 97 | 238903_at | LOC137886 | hypothetical protein LOC137886 | 8q11.23 | Consensussequence | Hs.99403.0 | AI636090 | Hs.99403.0.A1 |
| 98 | 204530_s_at | TOX | thymus high mobility group box protein TOX | 8q11.23 | Exemplarsequence | Hs.184297.0 | NM_014729.1 | g7662321 |
| 99 | 212449_s_at | LYPLA1 | lysophospholipase I | 8q11.23 | Consensussequence | Hs.12540.2 | BG288007 | Hs.12540.2_RC |
| 100 | 204301_at | KIAA0711 | KIAA0711 gene product | 8p23.2 | Exemplarsequence | Hs.5333.0 | NM_014867.1 | g7662259 |
| 101 | 232641_at | LOC169270 | hypothetical protein LOC169270 | 8p23.3 | Consensussequence | Hs.249181.0 | AC004908 | Hs.249181.0.S1 |
| 102 | 219340_s_at | CLN8 | ceroid-lipofuscinosis, neuronal 8 (epilepsy, | 8p23 | Exemplarsequence | Hs.127675.0 | AF123759.1 | g6467264 |
| | | | progressive with mental retardation) | | | | | |
| 103 | 210980_s_at | ASAH1 | N-acylsphingosine amidohydrolase (acid ceramidase) 1 | 8p22-p21.3 | Exemplarsequence | Hs.75811.1 | U47674.1 | g3860239 |
| 104 | 209997_x_at | PCM1 | pericentriolar material 1 | 8p22-p21.3 | Exemplarsequence | Hs.315478.0 | BC000453.1 | g12653366 |
| 105 | 213702_x_at | ASAH1 | N-acylsphingosine amidohydrolase (acid | 8p22-p21.3 | Consensussequence | Hs.75811.3 | AI934569 | Hs.75811.3.S1 |
| | | | ceramidase) 1 | | | | | |
| 106 | 209295_at | TNFRSF10B | tumor necrosis factor receptor superfamily, | 8p22-p21 | Exemplarsequence | Hs.51233.0 | AF016266.1 | g2529562 |
| | | | member 10b | | | | | |
| 107 | 206222_at | TNFRSF10C | tumor necrosis factor receptor superfamily, | 8p22-p21 | Exemplarsequence | Hs.119684.0 | NM_003841.1 | g10835042 |
| | | | member 10c, decoy without an intracellular | | | | | |
| | | | domain | | | | | |
| 108 | 212096_s_at | MTSG1 | transcription factor MTSG1 | 8p21.3 | Consensussequence | Hs.7946.0 | AL096842.1 | Hs.7946.0 |
| 109 | 203549_s_at | LPL | lipoprotein lipase | 8p22 | Exemplarsequence | Hs.180878.0 | NM_000237.1 | g4557726 |
| 110 | 222107_x_at | LZTS1 | leucine zipper, putative tumor suppressor 1 | 8p22 | Consensussequence | Hs.93605.6 | BE312985 | Hs.93605.6.S2 |
| 111 | 208231_at | NRG1 | neuregulin 1 | 8p21-p12 | Exemplarsequence | Hs.172816.0 | NM_013960.1 | g7669519 |
| 112 | 230746_s_at | STC1 | stanniocalcin 1 | 8p21-p11.2 | Consensussequence | Hs.25590.1 | AW003173 | Hs.25590.1.A1 |
| 113 | 211489_at | ADRA1A | adrenergic, alpha-1A-, receptor | 8p21-p11.2 | Exemplarsequence | Hs.52931.0 | D32201.1 | g927210 |
| 114 | 218777_at | FLJ22246 | hypothetical protein FLJ22246 | 8p21.2 | Exemplarsequence | Hs.289063.0 | NM_025232.1 | g13376835 |
| 115 | 205997_at | ADAM28 | a disintegrin and metalloproteinase domain 28 | 8p21.1 | Exemplarsequence | Hs.174030.0 | NM_021778.1 | g11496995 |
| 116 | 226179_at | | Homo sapiens, clone IMAGE: 5294823, | | Consensussequence | Hs.34549.0 | N63920 | Hs.34549.0.A1 |
| | | | mRNA | | | | | |
| 117 | 221123_x_at | LOC55893 | papillomavirus regulatory factor PRF-1 | 8p21.1 | Exemplarsequence | Hs.27410.0 | NM_018660.1 | g8923886 |
| 118 | 205770_at | GSR | glutathione reductase | 8p21.1 | Exemplarsequence | Hs.121524.0 | NM_000637.1 | g10835188 |
| 119 | 218149_s_at | DKFZp434K1210 | hypothetical protein DKFZp434K1210 | 8p21.1 | Exemplarsequence | Hs.32352.0 | NM_017606.1 | g8922146 |
| 120 | 235588_at | LOC157570 | hypothetical protein LOC157570 | 8p21.1 | Consensussequence | Hs.99480.0 | AA740849 | Hs.99480.0 |
| 121 | 236492_at | PPP2R2A | protein phosphatase 2 (formerly 2A), | 8p21.1 | Consensussequence | Hs.132822.0 | AI934447 | Hs.132822.0.A1 |
| | | | regulatory subunit B (PR 52), alpha isoform | | | | | |
| 122 | 212166_at | XPO7 | exportin 7 | 8p21 | Consensussequence | Hs.172685.0 | H38643 | Hs.172685.0 |
| 123 | 221478_at | BNIP3L | BCL2/adenovirus E1B 19 kDa interacting protein 3-like | 8p21 | Consensussequence | Hs.132955.0 | AL132665.1 | Hs.132955.0 |
| 124 | 220860_at | PURG | purine-rich element binding protein G | 8p11 | Exemplarsequence | Hs.278953.0 | NM_013357.1 | g7019506 |
| 125 | 211686_s_at | LOC84549 | RNA binding protein | 8p11.23 | Exemplarsequence | g13625185 | AF251062.1 | g13625185 |
| 126 | 200847_s_at | MGC8721 | hypothetical protein MGC8721 | 8p12 | Exemplarsequence | Hs.279921.0 | NM_016127.1 | g7706384 |
| 127 | 219897_at | FLJ12526 | hypothetical protein FLJ12526 | 8p11.23 | Exemplarsequence | Hs.151237.0 | NM_024787.1 | g13376151 |
| 128 | 231258_at | MGC8721 | hypothetical protein MGC8721 | 8p12 | Consensussequence | Hs.279921.2 | AV648367 | Hs.279921.2.S1 |
| 129 | 219624_at | BAG4 | BCL2-associated athanogene 4 | 8p11.21 | Exemplarsequence | Hs.194726.0 | NM_004874.1 | g6631074 |
| 130 | 207597_at | ADAM18 | a disintegrin and metalloproteinase domain 18 | 8p11.21 | Exemplarsequence | Hs.127930.0 | NM_014237.1 | g7656860 |
| 131 | 217819_at | LOC51125 | HSPC041 protein | 8p11.21 | Exemplarsequence | Hs.7953.0 | NM_016099.1 | g7705820 |
| 132 | 219292_at | FLJ10477 | hypothetical protein FLJ10477 | 8p11.1 | Exemplarsequence | Hs.7432.0 | NM_018105.1 | g8922445 |
| 133 | 235114_x_at | HOOK3 | hook3 protein | 8p11.1 | Consensussequence | Hs.130707.0 | N67300 | Hs.130707.0 |
| 134 | 209517_s_at | ASH2L | ash2 (absent, small, or homeotic)-like | 8p11.2 | Exemplarsequance | Hs.6856.1 | AB020982.1 | g4417209 |
| | | | (Drosophila) | | | | | |
| 135 | 216519_s_at | PROSC | proline synthetase co-transcribed homolog | 8p11.2 | Consensussequence | Hs.301959.3 | AK021923.1 | Hs.301959.3.S1 |
| | | | (bacterial) | | | | | |
| 136 | 214545_s_at | PROSC | proline synthetase co-transcribed homolog | 8p11.2 | Consensussequence | Hs.301959.2 | NM_007198.1 | Hs.301959.2.S1 |
| | | | (bacterial) | | | | | |
| 137 | 204817_at | ESPL1 | extra spindle poles like 1 (S. cerevisiae) | 8 | Exemplarsequence | Hs.153479.0 | NM_012291.1 | g6912453 |

| # | Sequence Source | Unigene_Accession | Cluster_Type | LocusLink | Full_Length_Reference_Seq |
|---|---|---|---|---|---|
| 1 | GenBank | Hs.273344 | fulllength | 25879 | NM_014156; NM_015420; DKFZP564O0463 protein |
| 2 | RefSeq | Hs.49 | fulllength | 4481 | NM_002445; macrophage scavenger receptor 1 isoform type 2 |
| | | | | | NM_138715; macrophage scavenger receptor 1 isoform type 1 |
| | | | | | NM_138716; macrophage scavenger receptor 1 isoform type 3 |
| 3 | RefSeq | Hs.169615 | fulllength | 65265 | NM_023080; hypothetical protein FLJ20989 |
| 4 | RefSeq | Hs.380935 | fulllength | 3297 | NM_005526; heat shock transcription factor 1 |
| 5 | GenBank | Hs.74562 | fulllength | 22827 | NM_014281; fuse-binding protein-interacting repressor isoform b |
| | | | | | NM_078480; fuse-binding protein-interacting repressor isoform a |
| 6 | GenBank | Hs.83727 | fulllength | 29894 | NM_013291; cleavage and polyadenylation specific factor 1, 160 kDa |
| 7 | GenBank | Hs.9812 | | | |
| 8 | GenBank | Hs.173094 | fulllength | 85453 | NM_033512; KIAA1750 protein |
| 9 | RefSeq | Hs.82129 | fulllength | 761 | NM_005181; carbonic anhydrase III |
| 10 | GenBank | Hs.4147 | fulllength | 23471 | NM_014294; translocating chain-associating membrane protein |
| 11 | RefSeq | Hs.78687 | fulllength | 8439 | NM_003580; neutral sphingomyelinase (N-SMase) activation |
| | | | | | associated factor |
| 12 | GenBank | Hs.406141 | fulllength | 137695 | NM_152417; hypothetical protein FLJ32370 |
| 13 | RefSeq | Hs.248146 | fulllength | 4342 | NM_005372; v-mos Moloney murine sarcoma viral oncogene homolog |
| 14 | GenBank | Hs.287478 | | | |
| 15 | GenBank | Hs.289293 | fulllength | 157695 | NM_175075; hypothetical protein INM01 |
| 16 | GenBank | Hs.48876 | fulllength | 2222 | NM_004462; farnesyl-diphosphate farnesyltransferase 1 |
| 17 | RefSeq | Hs.27842 | fulllength | 55326 | NM_018361; acid acyltransferase-epsilon |
| 18 | GenBank | Hs.356463 | fulllength | 2339 | NM_002027; farnesyltransferase, CAAX box, alpha |
| 19 | RefSeq | Hs.380963 | fulllength | 29883 | NM_013354; CCR4-NOT transcription complex, subunit 7 isoform |
| | | | | | 1 NM_054026; CCR4-NOT transcription complex, subunit 7 isoform 2 |
| 20 | RefSeq | Hs.75737 | fulllength | 5108 | NM_006197; pericentriolar material 1 |
| 21 | GenBank | Hs.75737 | fulllength | 5108 | NM_006197; pericentriolar material 1 |
| 22 | GenBank | Hs.75737 | fulllength | 5108 | NM_006197; pericentriolar material 1 |
| 23 | GenBank | Hs.71215 | fulllength | 9046 | NM_003974; docking protein 2, 56 kD |
| 24 | RefSeq | Hs.1274 | fulllength | 649 | NM_001199; bone morphogenetic protein 1 isoform 1, precursor |
| | | | | | NM_006128; bone morphogenetic protein 1 isoform 2, precursor |
| | | | | | NM_006129; bone morphogenetic protein 1 isoform 3, precursor |
| | | | | | NM_006130; bone morphogenetic protein 1 isoform 6, precursor |
| | | | | | NM_006131; bone morphogenetic protein 1 isoform 5, precursor |
| | | | | | NM_006132; bone morphogenetic protein 1 isoform 4, precursor |
| 25 | GenBank | Hs.27721 | fulllength | 54904 | NM_017778; WHSC1L1 protein isoform short NM_023034; |
| | | | | | WHSC1L1 protein isoform long |
| 26 | GenBank | Hs.443139 | est | | |
| 27 | RefSeq | Hs.318725 | fulllength | 51105 | NM_016018; CGI-72 protein |
| 28 | GenBank | Hs.323114 | fulllength | 79139 | NM_018630; NM_024295; hypothetical protein MGC3067 |
| 29 | GenBank | Hs.2463 | fulllength | 284 | NM_001146; angiopoietin 1 isoform a NM_139290; angiopoietin 1 |
| | | | | | isoform b |
| 30 | RefSeq | Hs.2463 | fulllength | 284 | NM_001146; angiopoietin 1 isoform a NM_139290; angiopoietin 1 |
| | | | | | isoform b |
| 31 | RefSeq | Hs.273344 | fulllength | 25879 | NM_014156; NM_015420; DKFZP564O0463 protein |
| 32 | GenBank | Hs.98471 | est | 137682 | NM_152416; hypothetical protein MGC40214 |
| 33 | GenBank | Hs.144027 | | | |
| 34 | GenBank | Hs.300592 | | 4603 | |
| 35 | RefSeq | Hs.55097 | fulllength | 28957 | NM_014018; mitochondrial ribosomal protein S28 |
| 36 | RefSeq | Hs.170198 | fulllength | 9650 | NM_014637; KIAA0009 gene product |
| 37 | GenBank | Hs.400259 | fulllength | 54984 | NM_017884; PIN2-interacting protein 1 |
| 38 | GenBank | Hs.71119 | fulllength | 7991 | NM_006765; Putative prostate cancer tumor suppressor isoform a |
| | | | | | NM_178234; Putative prostate cancer tumor suppressor isoform b |
| 39 | GenBank | Hs.131055 | | 286056 | |
| 40 | RefSeq | Hs.287624 | fulllength | 80094 | NM_025026; hypothetical protein FLJ14107 |
| 41 | GenBank | Hs.840 | fulllength | 3620 | NM_002164; indoleamine-pyrrole 2,3 dioxygenase |
| 42 | GenBank | Hs.170296 | | | |
| 43 | RefSeq | Hs.425311 | fulllength | 27257 | NM_014462; Lsm1 protein |
| 44 | GenBank | Hs.740 | fulllength | 5747 | NM_005607; PTK2 protein tyrosine kinase 2 isoform b |
| | | | | | NM_153831; PTK2 protein tyrosine kinase 2 isoform a |
| 45 | RefSeq | Hs.4742 | fulllength | 8733 | NM_003801; anchor attachment protein 1 |
| 46 | RefSeq | Hs.178551 | fulllength | 6132 | NM_000973; ribosomal protein L8 NM_033301; ribosomal protein |
| | | | | | L8 |
| 47 | GenBank | Hs.331601 | fulllength | 157542 | |
| 48 | GenBank | Hs.343589 | fulllength | 54512 | NM_019037; exosome complex exonuclease RRP41 |
| 49 | RefSeq | Hs.6459 | fulllength | 79581 | NM_024531; putative G-protein coupled receptor GPCR41 |
| 50 | RefSeq | Hs.43666 | fulllength | 11156 | NM_007079; protein tyrosine phosphatase type IVA, member 3 |
| | | | | | isoform 2 NM_032611; protein tyrosine phosphatase type IVA, member 3 isoform 1 |
| 51 | GenBank | Hs.239451 | fulllength | 2738 | NM_138465; GLI-Kruppel family member GLI4 |
| 52 | GenBank | Hs.380935 | fulllength | 3297 | NM_005526; heat shock transcription factor 1 |
| 53 | GenBank | Hs.149152 | fulllength | 114822 | NM_052924; rhophilin 1 |
| 54 | GenBank | Hs.10669 | fulllength | 50807 | |
| 55 | RefSeq | Hs.18029 | fulllength | 55093 | NM_018024; hypothetical protein FLJ10204 |
| 56 | RefSeq | Hs.323114 | fulllength | 79139 | NM_018630; NM_024295; hypothetical protein MGC3067 |
| 57 | GenBank | Hs.352376 | | | |
| 58 | GenBank | Hs.222088 | fulllength | 29028 | NM_014109; PRO2000 protein NM_032365; |
| 59 | GenBank | Hs.86970 | est | | |
| 60 | RefSeq | Hs.58189 | fulllength | 8667 | NM_003756; eukaryotic translation initiation factor 3, subunit 3 gamma, 40 kDa |
| 61 | GenBank | Hs.298229 | fulllength | 5202 | NM_012394; prefoldin 2 NM_080651; TRAP/Mediator complex |
| | | | | | component TRAP25 |
| 62 | GenBank | Hs.28285 | fulllength | 11236 | NM_007218; ring finger protein 139 |
| 63 | RefSeq | Hs.23019 | fulllength | 7564 | NM_006958; zinc finger protein 16 (KOX 9) |
| 64 | GenBank | Hs.81791 | fulllength | 4982 | NM_002546; osteoprotegerin precursor |
| 65 | GenBank | Hs.21034 | | 114788 | |
| 66 | GenBank | Hs.283740 | fulllength | 56943 | NM_020189; DC6 protein |
| 67 | GenBank | Hs.106673 | fulllength | 3646 | NM_001568; murine mammary tumor integration site 6 (oncogene |
| | | | | | homolog) |
| 68 | RefSeq | Hs.169395 | fulllength | 79870 | NM_024812; brain and acute leukemia, cytoplasmic |
| 69 | GenBank | Hs.290880 | | | |
| 70 | RefSeq | Hs.257924 | fulllength | 79977 | NM_024915; hypothetical protein FLJ13782 |
| 71 | RefSeq | Hs.197335 | fulllength | 10404 | NM_006102; NM_016134; plasma glutamate carboxypeptidase |
| 72 | GenBank | Hs.127331 | fulllength | 79815 | NM_024759; hypothetical protein FLJ13955 |
| 73 | GenBank | Hs.243901 | fulllength | | |
| 74 | GenBank | Hs.33074 | | | |
| 75 | GenBank | Hs.403869 | fulllength | 137392 | NM_145269; similar to CG6405 gene product |
| 76 | GenBank | Hs.116240 | est | | |
| 77 | RefSeq | Hs.22265 | fulllength | 54704 | NM_018444; pyruvate dehydrogenase phosphatase |
| 78 | GenBank | Hs.22265 | fulllength | 54704 | NM_018444; pyruvate dehydrogenase phosphatase |
| 79 | RefSeq | Hs.241384 | fulllength | 10646 | NM_006550; fibrinogen silencer binding protein |
| 80 | RefSeq | Hs.128501 | fulllength | 25788 | NM_012415; RAD54B homolog isoform 1 NM_134434; RAD54B |
| | | | | | homolog isoform 2 |
| 81 | RefSeq | Hs.144877 | fulllength | 9420 | NM_004820; cytochrome P450, family 7, subfamily B, polypeptide 1 |
| 82 | RefSeq | Hs.14158 | fulllength | 8895 | NM_003909; copine III |
| 83 | GenBank | Hs.2331 | fulllength | 1875 | NM_001951; E2F transcription factor 5 |
| 84 | RefSeq | Hs.171776 | fulllength | 3612 | NM_005536; inositol(myo)-1(or 4)-monophosphatase 1 |
| 85 | RefSeq | Hs.99519 | fulllength | 79752 | NM_024699; hypothetical protein FLJ14007 |
| 86 | GenBank | Hs.183861 | fulllength | 92421 | NM_152284; hypothetical protein MGC22825 |
| 87 | GenBank | Hs.42474 | fulllength | 9242 | NM_005098; musculin (activated B-cell factor-1) |
| 88 | RefSeq | Hs.1276 | fulllength | 661 | NM_001722; RNA polymerase III 53 kDa subunit RPC4 |
| 89 | GenBank | Hs.182364 | fulllength | 83690 | NM_031461; CocoaCrisp |
| 90 | GenBank | Hs.407120 | | | |
| 91 | RefSeq | Hs.153 | fulllength | 6129 | NM_000971; ribosomal protein L7 |
| 92 | GenBank | Hs.306681 | | | |
| 93 | GenBank | Hs.94631 | fulllength | 10565 | NM_006421; brefeldin A-inhibited guanine nucleotide-exchange protein 1 |
| 94 | GenBank | Hs.194562 | fulllength | 7013 | NM_003218; telomeric repeat binding factor 1 isoform 2 |
| | | | | | NM_017489; telomeric repeat binding factor 1 isoform 1 |
| 95 | RefSeq | Hs.287727 | fulllength | 80124 | NM_025054; valosin-containing protein (p97)/p47 complex- |
| | | | | | interacting protein p135 |
| 96 | GenBank | Hs.5518 | fulllength | 253943 | NM_152758; hypothetical protein FLJ31657 |
| 97 | GenBank | Hs.155572 | | 137886 | |
| 98 | RefSeq | Hs.184297 | fulllength | 9760 | NM_014729; thymus high mobility group box protein TOX |
| 99 | GenBank | Hs.12540 | fulllength | 10434 | NM_006330; lysophospholipase I |
| 100 | RefSeq | Hs.5333 | fulllength | 9920 | NM_014867; KIAA0711 gene product |
| 101 | GenBank | Hs.249181 | fulllength | 169270 | NM_173539; hypothetical protein LOC169270 |
| 102 | GenBank | Hs.127675 | fulllength | 2055 | NM_018941; CLN8 protein |
| 103 | GenBank | Hs.75811 | fulllength | 427 | NM_004315; N-acylsphingosine amidohydrolase (acid ceramidase) |
| | | | | | 1 isoform b NM_177924; N-acylsphingosine amidohydrolase (acid |
| | | | | | ceramidase) 1 preproprotein isoform a |
| 104 | GenBank | Hs.75737 | fulllength | 5108 | NM_006197; pericentriolar material 1 |
| 105 | GenBank | Hs.75811 | fulllength | 427 | NM_004315; N-acylsphingosine amidohydrolase (acid ceramidase) |
| | | | | | 1 isoform b NM_177924; N-acylsphingosine amidohydrolase (acid |
| | | | | | ceramidase) 1 preproprotein isoform a |
| 106 | GenBank | Hs.51233 | fulllength | 8795 | NM_003842; tumor necrosis factor receptor superfamily, member |
| | | | | | 10b isoform 1 precursor NM_147187; tumor necrosis factor |
| | | | | | receptor superfamily, member 10b isoform 2 precursor |
| 107 | RefSeq | Hs.119684 | fulllength | 8794 | NM_003841; tumor necrosis factor receptor superfamily, member |
| | | | | | 10c precursor |
| 108 | GenBank | Hs.7946 | fulllength | 57509 | NM_020749; transcription factor MTSG1 |
| 109 | RefSeq | Hs.180878 | fulllength | 4023 | NM_000237; lipoprotein lipase precursor |
| 110 | GenBank | Hs.93605 | fulllength | 11178 | NM_021020; leucine zipper, putative tumor suppressor 1 |
| 111 | RefSeq | Hs.172816 | fulllength | 3084 | NM_004495; neuregulin 1 isoform HRG-gamma NM_013956; |
| | | | | | neuregulin 1 isoform HRG-beta1 NM_013957; neuregulin 1 isoform |
| | | | | | HRG-beta2 NM_013958; neuregulin 1 isoform HRG-beta3 NM_013959; |
| | | | | | neuregulin 1 isoform SMDF NM_013960; neuregulin |
| | | | | | 1 isoform ndf43 NM_013961; neuregulin 1 isoform GGF NM_013962; |
| | | | | | neuregulin 1 isoform GGF2 NM_013964; neuregulin |
| | | | | | 1 isoform HRG-alpha |
| 112 | GenBank | Hs.25590 | fulllength | 6781 | NM_003155; stanniocalcin 1 |
| 113 | GenBank | Hs.52931 | fulllength | 148 | NM_000680; alpha-1A-adrenergic receptor isoform 1 NM_033302; |
| | | | | | alpha-1A-adrenergic receptor isoform 3 NM_033303; alpha-1A- |
| | | | | | adrenergic receptor isoform 2 NM_033304; alpha-1A-adrenergic |
| | | | | | receptor isoform 4 |
| 114 | RefSeq | Hs.289063 | fulllength | 80346 | NM_025232; hypothetical protein FLJ22246 |
| 115 | RefSeq | Hs.174030 | fulllength | 10863 | NM_014265; a disintegrin and metallaproteinase domain 28 |
| | | | | | isoform 1 preproprotein NM_021777; a disintegrin and |
| | | | | | metalloproteinase domain 28 isoform 3 preproprotein NM_021778; |
| | | | | | a disintegrin and metalloproteinase domain 28 isoform 2 |
| | | | | | preproprotein |
| 116 | GenBank | Hs.34549 | | | |
| 117 | RefSeq | Hs.27410 | fulllength | 55893 | NM_018660; papillomavirus regulatory factor PRF-1 |
| 118 | RefSeq | Hs.193974 | fulllength | 2936 | NM_000637; glutathione reductase |
| 119 | RefSeq | Hs.32352 | fulllength | 54775 | NM_017606; hypothetical protein DKFZp434K1210 |
| 120 | GenBank | Hs.99480 | | 157570 | |
| 121 | GenBank | Hs.179574 | fulllength | 5520 | NM_002717; protein phosphatase 2 (formerly 2A), regulatory |
| | | | | | subunit B (PR 52), alpha isoform |
| 122 | GenBank | Hs.172685 | fulllength | 23039 | NM_015024; exportin 7 |
| 123 | GenBank | Hs.132955 | fulllength | 665 | NM_004331; BCL2/adenovirus E1B 19 kD-interacting protein 3-like |
| 124 | RefSeq | Hs.278953 | fulllength | 29942 | NM_013357; purine-rich element binding protein G |
| 125 | GenBank | Hs.77135 | fulllength | 84549 | NM_032509; RNA binding protein |
| 126 | RefSeq | Hs.279921 | fulllength | 51669 | NM_016127; hypothetical protein MGC8721 |
| 127 | RefSeq | Hs.151237 | fulllength | 79845 | NM_024787; hypothetical protein FLJ12526 |
| 128 | GenBank | Hs.279921 | fulllength | 51669 | NM_016127; hypothetical protein MGC8721 |
| 129 | RefSeq | Hs.194726 | fulllength | 9530 | NM_004874; BCL2-associated athanogene 4 |
| 130 | RefSeq | Hs.127930 | fulllength | 8749 | NM_014237; a disintegrin and metalloproteinase domain 18 |
| | | | | | preproprotein |
| 131 | RefSeq | Hs.7953 | fulllength | 51125 | NM_016099; HSPC041 protein |
| 132 | RefSeq | Hs.7432 | fulllength | 55145 | NM_018105; hypothetical protein FLJ10477 |
| 133 | GenBank | Hs.130707 | fulllength | 84376 | NM_032410; hook3 protein |
| 134 | GenBank | Hs.6856 | fulllength | 9070 | NM_004674; ash2 (absent, small, or homeotic)-like |
| 135 | GenBank | Hs.301959 | fulllength | 11212 | NM_007198; proline synthetase co-transcribed homolog |
| 136 | GenBenk | Hs.301959 | fulllength | 11212 | NM_007198; proline synthetase co-transcribed homolog |
| 137 | RefSeq | Hs.153479 | fulllength | 9700 | NM_012291; extra spindle poles like 1 |

TABLE 27
11q23/MLL

| # | affy id | HUGO name | F | p | q | Title | MapLocation | Sequence Type |
|---|---|---|---|---|---|---|---|---|
| 1 | 223503_at | DKFZP566N034 | 78.82 | 2.41e−08 | 2.11e−04 | hypothetical protein DKFZp566N034 | 2q21.2 | Exemplarsequence |
| 2 | 202697_at | CPSF5 | 56.52 | 3.27e−09 | 5.72e−05 | cleavage and polyadenylation specific factor 5, 25 kDa | 16q12.1 | Exemplarsequence |
| 3 | 210106_at | RDH5 | 54.92 | 4.34e−09 | 5.72e−05 | retinol dehydrogenase 5 (11-cis and 9-cis) | 12q13-q14 | Exemplarsequence |
| 4 | 218977_s_at | SECP43 | 48.90 | 1.01e−06 | 5.31e−03 | tRNA selenocysteine associated protein | 1p35.2 | Exemplarsequence |
| 5 | 206290_s_at | RGS7 | 41.72 | 1.65e−05 | 1.61e−02 | regulator of G-protein signalling 7 | 1q43 | Exemplarsequence |
| 6 | 205257_s_at | AMPH | 40.30 | 2.99e−06 | 7.17e−03 | amphiphysin (Stiff-Man syndrome with breast cancer 128 kDa autoantigen) | 7p14-p13 | Exemplarsequence |
| 7 | 236200_at | | 37.71 | 7.83e−05 | 3.75e−02 | ESTs | | Consensussequence |
| 8 | 203345_s_at | M96 | 36.71 | 2.72e−06 | 7.17e−03 | likely ortholog of mouse metal response element binding transcription factor 2 | 1p22.1 | Consensussequence |
| 9 | 202320_at | GTF3C1 | 35.31 | 7.14e−07 | 4.71e−03 | general transcription factor IIIC, polypeptide 1, alpha 220 kDa | 16p12 | Exemplarsequence |
| 10 | 238315_s_at | MGC45586 | 35.21 | 4.66e−06 | 8.77e−03 | hypothetical protein MGC45586 | 19q13.12 | Consensussequence |
| 11 | 205396_at | DKFZP586N0721 | 33.58 | 7.64e−06 | 1.17e−02 | DKFZP586N0721 protein | 15q22.2 | Consensussequence |
| 12 | 212570_at | KIAA0830 | 32.04 | 8.45e−05 | 3.91e−02 | KIAA0830 protein | 11q21 | Consensussequence |
| 13 | 218444_at | ALG12 | 31.97 | 1.79e−06 | 7.10e−03 | dolichyl-p-mannose:Man7GlcNAc2-PP-dolichyl | | Exemplarsequence |
| | | | | | | mannosyltransferase | | |
| 14 | 201613_s_at | RUVBL1 | 30.44 | 3.84e−06 | 7.80e−03 | RuvB-like 1 (E. coli) | 3q21 | Exemplarsequence |
| 15 | 222891_s_at | BCL11A | 29.90 | 2.20e−06 | 7.10e−03 | B-cell CLL/lymphoma 11A (zinc finger protein) | 2p15 | Consensussequence |
| 16 | 225129_at | CPNE2 | 29.81 | 2.32e−06 | 7.10e−03 | copine II | 16q12.2 | Consensussequence |
| 17 | 202371_at | FLJ21174 | 29.54 | 2.42e−06 | 7.10e−03 | hypothetical protein FLJ21174 | Xq22.1 | Exemplarsequence |
| 18 | 215153_at | CAPON | 29.54 | 4.59e−04 | 8.12e−02 | C-terminal PDZ domain ligand of neuronal nitric oxide | 1q23.1 | Consensussequence |
| | | | | | | synthase | | |
| 19 | 220849_at | FLJ22659 | 29.15 | 5.16e−04 | 8.36e−02 | hypothetical protein FLJ22659 | 17p11.2 | Exemplarsequence |
| 20 | 212746_s_at | KIAA0470 | 28.61 | 3.65e−06 | 7.80e−03 | KIAA0470 gene product | 1q44 | Consensussequence |
| 21 | 217359_s_at | NCAM1 | 38196 | 1.98e−04 | 5.68e−02 | neural cell adhesion molecule 1 | 11q23.1 | Consensussequence |
| 22 | 227769_at | GPR27 | 27.26 | 5.46e−06 | 9.41e−03 | G protein-coupled receptor 27 | 3p21-p14 | Consensussequence |
| 23 | 209620_s_at | ABCB7 | 38287 | 5.71e−06 | 9.41e−03 | ATP-binding cassette, sub-family B (MDR/TAP), member 7 | Xq12-q13 | Exemplarsequence |
| 24 | 204886_at | STK18 | 26.85 | 1.59e−05 | 1.61e−02 | serine/threonine kinase 18 | 4q27-q28 | Consensussequence |
| 25 | 208519_x_at | GNRH2 | 26.78 | 1.02e−04 | 4.01e−02 | gonadotropin-releasing hormone 2 | 20p13 | Exemplarsequence |
| 26 | 221077_at | FLJ10376 | 26.31 | 8.28e−06 | 1.17e−02 | hypothetical protein FLJ10376 | 10p12.1 | Exemplarsequence |
| 27 | 215160_x_at | FRG1 | 38317 | 2.67e−05 | 2.19e−02 | FSHD region gene 1 | 4q35 | Consensussequence |
| 28 | 59625_at | LOC283849 | 38194 | 9.56e−06 | 1.20e−02 | hypothetical protein LOC283849 | 16q21 | Consensussequence |
| 29 | 214743_at | CUTL1 | 38194 | 8.44e−06 | 1.17e−02 | cut-like 1, CCAAT displacement protein (Drosophila) | 7q22 | Consensussequence |
| 30 | 218274_s_at | FLJ10415 | 25.93 | 2.34e−05 | 2.06e−02 | hypothetical protein FLJ10415 | 2q36.1 | Exemplarsequence |
| 31 | 201578_at | PODXL | 25.92 | 2.67e−04 | 6.88e−02 | podocalyxin-like | 7q32-q33 | Exemplarsequence |
| 32 | 206310_at | SPINK2 | 25.68 | 1.02e−05 | 1.21e−02 | serine protease inhibitor, Kazal type, 2 (acrosin-trypsin | 4q12 | Exemplarsequence |
| | | | | | | inhibitor) | | |
| 33 | 219555_s_at | BM039 | 25.65 | 2.75e−05 | 2.19e−02 | uncharacterized bone marrow protein BM039 | 16q23.1 | Exemplarsequence |
| 34 | 200844_s_at | AOP2 | 25.51 | 5.34e−05 | 3.09e−02 | antioxidant protein 2 | 1q23.3 | Consensussequence |
| 35 | 203484_at | SEC61G | 25.38 | 9.45e−05 | 4.01e−02 | Sec61 gamma | 7p11.2 | Exemplarsequence |
| 36 | 207185_at | SLC10A1 | 25.31 | 6.43e−04 | 9.37e−02 | solute carrier family 10 (sodium/bile acid cotransporter | 14q24.1 | Exemplarsequence |
| | | | | | | family), member 1 | | |
| 37 | 206295_at | IL18 | 25.27 | 9.31e−06 | 1.20e−02 | interleukin 18 (interferon-gamma-inducing factor) | 11q22.2-q22.3 | Exemplarsequence |
| 38 | 244795_at | | 38255 | 9.52e−05 | 4.01e−02 | ESTs | | Consensussequence |
| 39 | 201709_s_at | NIPSNAP1 | 38224 | 3.67e−05 | 2.55e−02 | nipsnap homolog 1 (C. elegans) | 22q12.2 | Exemplarsequence |
| 40 | 201869_s_at | TBL1X | 24.95 | 1.08e−05 | 1.21e−02 | transducin (beta)-like 1X-linked | Xp22.3 | Consensussequence |
| 41 | 201936_s_at | EIF4G3 | 24.88 | 1.10e−05 | 1.21e−02 | eukaryotic translation initiation factor 4 gamma, 3 | 1p36.12 | Exemplarsequence |
| 42 | 217081_at | | 24.63 | 4.66e−04 | 8.15e−02 | | | Consensussequence |
| 43 | 202300_at | HBXIP | 24.42 | 1.70e−04 | 5.15e−02 | hepatitis B virus x interacting protein | 1p13.1 | Exemplarsequence |
| 44 | 206298_at | LOC58504 | 24.13 | 1.58e−05 | 1.61e−02 | hypothetical protein from clones 23549 and 23762 | 10q11.21 | Exemplarsequence |
| 45 | 228113_at | STAT3 | 23.89 | 1.72e−05 | 1.62e−02 | signal transducer and activator of transcription 3 (acute− | 17q21 | Consensussequence |
| | | | | | | phase response factor) | | |
| 46 | 230001_at | LOC92979 | 23.70 | 9.50e−04 | 0.11 | hypothetical protein BC009489 | 12q13.13 | Consensussequence |
| 47 | 205374_at | SLN | 23.45 | 3.99e−04 | 7.53e−02 | sarcolipin | 11q22-q23 | Exemplarsequence |
| 48 | 203938_s_at | TAF1C | 23.38 | 2.88e−04 | 6.88e−02 | TATA box binding protein (TBP)-associated factor, RNA | 16q24 | Exemplarsequence |
| | | | | | | polymerase I, C, 110 kDa | | |
| 49 | 212015_x_at | PTBP1 | 23.36 | 1.16e−04 | 4.01e−02 | polypyrimidine tract binding protein 1 | 19p13.3 | Consensussequence |
| 50 | 211471_s_at | RAB36 | 23.25 | 9.36e−04 | 0.11 | RAB36, member RAS oncogene family | 22q11.22 | Exemplarsequence |

| | | Sequence | | | | | | |
|---|---|---|---|---|---|---|---|---|
| # | Transcript ID | Derived From | Sequence ID | Sequence Source | Unigene_Accession | Cluster_Type | LocusLink | Full_Length_Reference_Seq |
| 1 | Hs.331633.0 | AF255647.1 | g12005818 | GenBank | Hs.331633 | fulllength | 81615 | NM_030923; hypothetical protein DKFZp566N034 |
| 2 | Hs.9605.0 | NM_007006.1 | g5901925 | RefSeq | Hs.9605 | fulllength | 11051 | NM_007006; cleavage and polyadenylation specific factor 5, 25 kD subunit |
| 3 | Hs.172914.0 | U43559.1 | g1616653 | GenBank | Hs.172914 | fulllength | 5959 | NM_002905; retinol dehydrogenase 5 (11-cis and 9- |
| | | | | | | | | cis) |
| 4 | Hs.266935.0 | NM_017846.1 | g8923459 | RefSeq | Hs.266935 | fulllength | 54952 | NM_017846; tRNA selenocysteine associated |
| | | | | | | | | protein |
| 5 | Hs.79348.0 | NM_002924.1 | g11140808 | RefSeq | Hs.79348 | fulllength | 6000 | NM_002924; regulator of G-protein signalling 7 |
| 6 | Hs.173034.0 | NM_001635.1 | g4502080 | RefSeq | Hs.173034 | fulllength | 273 | NM_001635; amphiphysin isoform 1 NM_139316; |
| | | | | | | | | amphiphysin isoform 2 |
| 7 | Hs.12899.0 | AI698159 | Hs.12899.0.A1 | GenBank | Hs.12899 | est | | |
| 8 | Hs.31016.0 | AI566096 | Hs.31016.0.A1 | GenBank | Hs.31016 | fulllength | 22823 | NM_007358; putative DNA binding protein |
| 9 | Hs.3310 | NM_001520.1 | g4753160 | RefSeq | Hs.331 | fulllength | 2975 | NM_001520; general transcription factor IIIC, |
| | | | | | | | | polypeptide 1, alpha 220 kDa |
| 10 | Hs.199359.0 | BF061829 | Hs.199359.0.A1 | GenBank | Hs.145473 | est | 163081 | NM_152603: hypothetical protein MGC45586 |
| 11 | Hs.211578.0 | BF971416 | Hs.211578.0 | GenBank | Hs.99843 | fulllength | 25856 | NM_015400; DKFZP586N0721 protein |
| 12 | Hs.167115.0 | AL573201 | Hs.167115.0.S1 | Genbank | Hs.167115 | fulllength | 23052 | |
| 13 | Hs.77575.0 | NM_024105.1 | g13129113 | RefSeq | Hs.77575 | fulllength | 79087 | NM_024105; dolichyl-p-mannose:Man7GlcNAc2-PP- |
| | | | | | | | | dolichyl mannosyltransferase |
| 14 | Hs.272822.0 | BC000519.1 | g12653494 | GenBank | Hs.272822 | fulllength | 8607 | NM_003707; TATA binding protein interacting |
| | | | | | | | | protein 49 kDa |
| 15 | Hs.130881.0 | AI912275 | Hs.130881.0.S2 | GenBank | Hs.130881 | fulllength | 53335 | NM_018014; B-cell CLL/lymphoma 11A isoform 2 |
| | | | | | | | | NM_022893; B-cell CLL/lymphoma 11A isoform 1 |
| | | | | | | | | NM_138552; B-cell CLL/lymphoma 11A isoform 4 |
| | | | | | | | | NM_138553; B-cell CLL/lymphoma 11A isoform 5 |
| | | | | | | | | NM_138559; B-cell CLL/lymphoma 11A isoform 3 |
| 16 | Hs.96144.0 | AW170571 | Hs.96144.0.A1 | GenBank | Hs.339809 | fulllength | 221184 | NM_152727; copine II |
| 17 | Hs.194329.0 | NM_024863.1 | g13376293 | RefSeq | Hs.194329 | fulllength | 79921 | NM_024863; hypothetical protein FLJ21174 |
| 18 | Hs.129729.0 | AF037070.1 | Hs.129729.0 | GenBank | Hs.129729 | | 9722 | NM_014697; C-terminal PDZ domain ligand of |
| | | | | | | | | neuronal nitric oxide synthase |
| 19 | Hs.276833.0 | NM_024934.1 | g13376405 | RefSeq | Hs.276833 | fulllength | 79999 | NM_024934; hypothetical protein FLJ22659 |
| 20 | Hs.25132.3 | AA126789 | Hs.25132.3_RC | GenBank | Hs.25132 | fulllength | 9859 | NM_014812; KARP-1-binding protein |
| 21 | Hs.167988.3 | M22094.1 | Hs.167988.3 | GenBank | Hs.167988 | fulllength | 4684 | NM_000615; neural cell adhesion molecule 1 |
| 22 | Hs.250899.1 | AI703476 | Hs.250899.1.A1 | GenBank | Hs.356084 | fulllength | 2850 | NM_018971; G protein-coupled receptor 27 |
| 23 | Hs.125856.0 | AB005289.1 | g3228278 | GenBank | Hs.125856 | fulllength | 22 | NM_004299; ATP-binding cassette, sub-family B, |
| | | | | | | | | member 7 |
| 24 | Hs.172052.0 | AL043646 | Hs.172052.0 | GenBank | Hs.172052 | fulllength | 10733 | NM_014264; serine/threonine kinase 18 |
| 25 | Hs.129715.0 | NM_001501.1 | g4504056 | RefSeq | Hs.129715 | fulllength | 2797 | NM_001501; gonadotropin-releasing hormone 2 |
| | | | | | | | | isoform a preproprotein NM_178331; gonadotropin- |
| | | | | | | | | releasing hormone 2 isoform c preproprotein |
| | | | | | | | | NM_178332; gonadotropin-releasing hormone 2 |
| | | | | | | | | isoform b preproprotein |
| 26 | Hs.202289.0 | NM_018076.1 | g8922385 | RefSeq | Hs.333126 | fulllength | 55130 | NM_018076; |
| 27 | Hs.203772.1 | AL441988 | Hs.203772.1.S1 | GenBank | Hs.203772 | fulllength | 2483 | NM_004477; FSHD region gene 1 |
| 28 | 4854714_rc | AI912351 | 4854714_rc | GenBank | Hs.278439 | fulllength | 283849 | NM_003946; nucleolar protein 3 (apoptosis |
| | | | | | | | | repressor with CARD domain) NM_178516; |
| | | | | | | | | hypothetical protein LOC283849 |
| 29 | Hs.147049.1 | BE046521 | Hs.147049.1 | GenBank | Hs.147049 | fulllength | 1523 | NM_001913; cut-like 1, CCAAT displacement |
| | | | | | | | | protein isoform b NM_181500; NM_181552; |
| 30 | Hs.23788.0 | NM_018089.1 | g8922412 | RefSeq | Hs.23788 | fulllength | 55139 | NM_018089; hypothetical protein FLJ10415 |
| 31 | Hs.16426.0 | NM_005397.1 | g4885556 | RefSeq | Hs.16426 | fulllength | 5420 | NM_005397; podocalyxin-like |
| 32 | Hs.98243.0 | NM_021114.1 | g10863910 | RefSeq | Hs.98243 | fulllength | 6691 | NM_021114; serine protease inhibitor, Kazal type, 2 |
| | | | | | | | | (acrosin-trypsin inhibitor) |
| 33 | Hs.283532.0 | NM_018455.1 | g8922096 | RefSeq | Hs.283532 | fulllength | 55839 | NM_018455; uncharacterized bone marrow protein |
| | | | | | | | | BM039 |
| 34 | Hs.120.0 | BE869583 | Hs.120.0_RC | GenBank | Hs.120 | fulllength | 9588 | NM_004905; peroxiredoxin 6 |
| 35 | Hs.9950.0 | NM_014302.1 | g7657545 | RefSeq | Hs.9950 | fulllength | 23480 | NM_014302; Sec61 gamma |
| 36 | Hs.952.0 | NM_003049.1 | g4506970 | RefSeq | Hs.952 | fulllength | 6554 | NM_003049; solute carrier family 10 (sodium/bile |
| | | | | | | | | acid cotransporter family), member 1 |
| 37 | Hs.83077.0 | NM_001562.1 | g4504652 | RefSeq | Hs.83077 | fulllength | 3606 | NM_001562; interleukin 18 proprotein |
| 38 | Hs.176950.0 | AV693986 | Hs.176950.0.A1 | GenBank | Hs.176950 | est | | |
| 39 | Hs.173878.0 | NM_003634.1 | g4505398 | RefSeq | Hs.173878 | fulllength | 8508 | NM_003634; NIPSNAP homolog 1 |
| 40 | Hs.76536.0 | NM_005647.1 | Hs.76536.0_RC | GenBank | Hs.76536 | fulllength | 6907 | NM_005647; transducin beta-like 1X |
| 41 | Hs.25732.0 | NM_003760.2 | g10092600 | RefSeq | Hs.25732 | fulllength | 8672 | NM_003760; eukaryotic translation initiation factor 4 |
| | | | | | | | | gamma, 3 |
| 42 | Hs.194767.0 | AL031983 | Hs.194767.0.S1 | GenBank | | | | |
| 43 | Hs.80464.0 | NM_006402.1 | g5454169 | RefSeq | Hs.433355 | fulllength | 10542 | NM_006402; hepatitis B virus x-interacting |
| | | | | | | | | protein |
| 44 | Hs.87241.0 | NM_021226.1 | g10864038 | RefSeq | Hs.87241 | fulllength | 58504 | NM_021226; hypothetical protein from clones 23549 |
| | | | | | | | | and 23762 |
| 45 | Hs.321677.1 | R62453 | Hs.321677.1 | GenBank | Hs.321677 | fulllength | 6774 | NM_003150; signal transducer and activator of |
| | | | | | | | | transcription 3 isoform 2 NM_139276; signal |
| | | | | | | | | transducer and activator of transcription 3 isoform 1 |
| | | | | | | | | NM_175738; RAB37, member of RAS oncogene |
| | | | | | | | | family |
| 46 | Hs.61969.0 | AI807693 | Hs.61969.0.A1 | GenBank | Hs.65377 | fulllength | 92979 | NM_138396; hypothetical protein BC009489 |
| 47 | Hs.15219.0 | NM_003063.1 | g4507062 | RefSeq | Hs.334629 | fulllength | 6588 | NM_003063; sarcolipin NM_032697; |
| 48 | Hs.153022.0 | NM_005679.1 | g5032144 | RefSeq | Hs.153022 | fulllength | 9013 | NM_005679; TBP-associated factor 1C isoform 1 |
| | | | | | | | | NM_139353; TBP-associated factor 1C isoform 2 |
| 49 | Hs.172550.3 | AA679988 | Hs.172550.3.S1 | GenBank | Hs.172550 | fulllength | 5725 | NM_002819; polypyrimidine tract-binding protein 1 |
| | | | | | | | | isoform a NM_031990; polypyrimidine tract-binding |
| | | | | | | | | protein 1 isoform b NM_031991; polypyrimidine tract |
| | | | | | | | | binding protein 1 isoform c NM_175847; |
| | | | | | | | | polypyrimidine tract-binding protein 1 isoform d |
| 50 | Hs.38772.1 | AF133588.1 | g6049163 | Genbank | Hs.38772 | fulllength | 9609 | NM_004914; RAB36, member RAS oncogene family |

TABLE 28
11q23/MLL

| # | affy id | HUGO name | Title | MapLocation | Sequence Type | Transcript ID | Sequence Derived From | Sequence ID |
|---|---|---|---|---|---|---|---|---|
| 1 | 213122_at | KIAA1750 | KIAA1750 protein | 8q22.1 | Consensussequence | Hs.173094.0 | AI096375 | Hs.173094.0.S1 |
| 2 | 201398_s_at | TRAM | translocating chain-associating membrane protein | 8q13.1 | Exemplarsequence | Hs.4147.0 | BC000687.1 | g12653796 |
| 3 | 205608_s_at | ANGPT1 | angiopoietin 1 | 8q22.3-q23 | Exemplarsequence | Hs.2463.0 | U83508.1 | g1907326 |
| 4 | 205609_at | ANGPT1 | angiopoietin 1 | 8q22.3-q23 | Exemplarsequence | Hs.2463.0 | NM_001146.1 | g4502086 |
| 5 | 220843_s_at | DKFZP564O0463 | DKFZP564O0463 protein | 8q22.3 | Exemplarsequence | Hs.273344.0 | NM_014156.1 | g7661767 |
| 6 | 235509_at | MGC40214 | hypothetical protein MGC40214 | 8q22.1 | Consensussequence | Hs.98471.0 | AV662196 | Hs.98471.0_RC |
| 7 | 203208_s_at | CHPPR | likely ortholog of chicken chondrocyte protein with a poly-proline | 8q12.1 | Exemplarsequence | Hs.170198.0 | NM_014637.1 | g7661853 |
| | | | region | | | | | |
| 8 | 230977_at | LOC286056 | hypothetical protein LOC286056 | 8p21.2 | Consensussequence | Hs.131055.0 | AI016313 | Hs.131055.0.A1 |
| 9 | 203534_at | LSM1 | LSM1 homolog, U6 small nuclear RNA | 8p11.2 | Exemplarsequence | Hs.111783.0 | NM_014462.1 | g7657312 |
| | | | associated (S. cerevisiae) | | | | | |
| 10 | 209510_at | TRC8 | patched related protein translocated in renal cancer | 8q24 | Exemplarsequence | Hs.28285.0 | AF064801.1 | g3395786 |
| 11 | 218899_s_at | BAALC | brain and acute leukemia, cytoplasmic | 8q22.3 | Exemplarsequence | Hs.169395.0 | NM_024812.1 | g13376199 |
| 12 | 220549_at | FSBP | fibrinogen silencer binding protein | 8q22.1 | Exemplarsequence | Hs.241384.0 | NM_006550.1 | g5729829 |
| 13 | 209295_at | TNFRSF10B | tumor necrosis factor receptor | 8p22-p21 | Exemplarsequence | Hs.51233.0 | AF016266.1 | g2529562 |
| | | | superfamily, member 10b | | | | | |
| 14 | 212096_s_at | MTSG1 | transcription factor MTSG1 | 8p21.3 | Consensussequence | Hs.7946.0 | AL096842.1 | Hs.7946.0 |
| 15 | 230746_s_at | STC1 | stanniocalcin 1 | 8p21-p11.2 | Consensussequence | Hs.25590.1 | AW003173 | Hs.25590.1.A1 |
| 16 | 205997_at | ADAM28 | a disintegrin and metalloproteinase | 8p21.1 | Exemplarsequence | Hs.174030.0 | NM_021778.1 | g11496995 |
| | | | domain 28 | | | | | |
| 17 | 221123_x_at | LOC55893 | papillomavirus regulatory factor PRF-1 | 8p21.1 | Exemplarsequence | Hs.27410.0 | NM_018660.1 | g8923886 |
| 18 | 204817_at | ESPL1 | extra spindle poles like 1 (S. cerevisiae) | 8 | Exemplarsequence | Hs.153479.0 | NM_012291.1 | g6912453 |
| 19 | 224218_s_at | TRPS1 | trichorhinophalangeal syndrome I | 8q24.12 | Exemplarsequence | Hs.26102.1 | AF264784.1 | g10644121 |
| 20 | 218692_at | FLJ20366 | hypothetical protein FLJ20366 | 8q23.2 | Exemplarsequence | Hs.8358.0 | NM_017786.1 | g8923340 |
| 21 | 230016_at | | Homo sapiens cDNA FLJ13277 fis, clone | | Consensussequence | Hs.55043.0 | AU155118 | Hs.55043.0.S1 |
| | | | OVARC1001044. | | | | | |
| 22 | 222199_s_at | BIN3 | bridging integrator 3 | 8p21.2 | Consensussequence | Hs.68090.1 | AK001289.1 | Hs.68090.1.S1 |
| 23 | 232693_s_at | LOC55893 | papillomavirus regulatory factor PRF-1 | 8p21.1 | Consensussequence | Hs.27410.2 | AK021850.1 | Hs.27410.2.S1 |
| 24 | 223216_s_at | LOC55893 | papillomavirus regulatory factor PRF-1 | 8p21.1 | Exemplarsequence | Hs.27410.1 | BC001237.1 | g12654788 |
| 25 | 212865_s_at | COL14A1 | collagen, type XIV, alpha 1 (undulin) | 8q23 | Consensussequence | Hs.36131.0 | BF449063 | Hs.36131.0.S1 |
| 26 | 239860_at | | ESTs | | Consensussequence | Hs.16292.0 | AI311917 | Hs.16292.0.A1 |
| 27 | 203207_s_at | CHPPR | likely ortholog of chicken chondrocyte | 8q12.1 | Consensussequence | Hs.170198.0 | BF214329 | Hs.170198.0.S1 |
| | | | protein with a poly-proline region | | | | | |
| 28 | 219665_at | FLJ22494 | hypothetical protein FLJ22494 | 8p21.2 | Exemplarsequence | Hs.170132.0 | NM_024815.1 | g13376205 |
| 29 | 224316_at | FLJ20038 | hypothetical protein FLJ20038 | 8p21.1 | Exemplarsequence | Hs.72071.1 | AF130091.1 | g11493486 |
| 30 | 231764_at | CHRAC1 | chromatin accessibility complex 1 | 8q24.3 | Consensussequence | Hs.279704.0 | AK023537.1 | Hs.279704.0 |
| 31 | 219071_x_at | LOC51236 | hypothetical protein LOC51236 | 8q24.3 | Exemplarsequence | Hs.300224.0 | NM_016458.2 | g13124772 |
| 32 | 226707_at | PP3856 | similar to CG3714 gene product | 8q24.3 | Consensussequence | Hs.9614.3 | BE870868 | Hs.9614.3_RC |
| 33 | 226129_at | | Homo sapiens, clone IMAGE: 5455669, | | Consensussequence | Hs.67776.0 | AI949095 | Hs.67776.0.A1 |
| | | | mRNA, partial cds | | | | | |
| 34 | 212149_at | KIAA0143 | KIAA0143 protein | 8q24.22 | Consensussequence | Hs.84087.0 | AA805651 | Hs.84087.0.S1 |
| 35 | 212150_at | KIAA0143 | KIAA0143 protein | 8q24.22 | Consensussequence | Hs.84087.0 | AA805651 | Hs.84087.0.S1 |
| 36 | 234351_x_at | TRPS1 | trichorhinophalangeal syndrome I | 8q24.12 | Consensussequence | Hs.26102.2 | AK000948.1 | Hs.26102.2 |
| 37 | 212157_at | SDC2 | syndecan 2 (heparan sulfate proteoglycan | 8q22-q23 | Consensussequence | Hs.1501.0 | J04621.1 | Hs.1501.0 |
| | | | 1, cell surface-associated, fibroglycan) | | | | | |
| 38 | 214485_at | ODF1 | outer dense fiber of sperm tails 1 | 8q22.3 | Consensussequence | Hs.159274.0 | NM_024410.1 | Hs.159274.0 |
| 39 | 220400_at | FLJ20583 | hypothetical protein FLJ20583 | 8q22.1 | Exemplarsequence | Hs.158836.0 | NM_017890.1 | g8923544 |
| 40 | 212248_at | | Homo sapiens, Similar to LYRIC, clone | | Consensussequence | Hs.243901.0 | AI972475 | Hs.243901.0.S1 |
| | | | MGC: 41931 IMAGE: 5298467, mRNA, complete cds | | | | | |
| 41 | 232023_at | MGC26979 | hypothetical protein MGC26979 | 8q21.3 | Consensussequence | Hs.130554.0 | AL575584 | Hs.130554.0 |
| 42 | 205529_s_at | CBFA2T1 | core-binding factor, runt domain, alpha | 8q22 | Exemplarsequence | Hs.31551.0 | NM_004349.1 | g4757915 |
| | | | subunit 2; translocated to, 1; cyclin D-related | | | | | |
| 43 | 200062_s_at- | RPL30 | ribosomal protein L30 | 8q22 | Exemplarsequence | Hs.111222.0 | L05095.1 | g388034 |
| | HG-U133A | | | | | | | |
| 44 | 219793_at | SNX16 | sorting nexin 16 | 8q21.12 | Exemplarsequence | Hs.128645.0 | NM_022133.1 | g11545864 |
| 45 | 225622_at | PAG | phosphoprotein associated with | 8q21.11 | Consensussequence | Hs.266175.0 | NM_018440.1 | Hs.266175.0 |
| | | | glycosphingolipid-enriched microdomains | | | | | |
| 46 | 214161_at | C8orf1 | chromosome 8 open reading frame 1 | 8q21 | Consensussequence | Hs.40539.1 | BF057458 | Hs.40539.1.A1 |
| 47 | 44783_s_at | HEY1 | hairy/enhancer-of-split related with YRPW | 8q21 | Consensussequence | 4838473 | R61374 | 4838473 |
| | | | motif 1 | | | | | |
| 48 | 205949_at | CA1 | carbonic anhydrase I | 8q13-q22.1 | Exemplarsequence | Hs.23118.0 | M33987.1 | g179792 |
| 49 | 202824_s_at | TCEB1 | transcription elongation factor B (SIII), | 8q13.3 | Exemplarsequence | Hs.184693.0 | NM_005648.1 | g5032160 |
| | | | polypeptide 1 (15 kDa, elongin C) | | | | | |
| 50 | 205731_s_at | NCOA2 | nuclear receptor coactivator 2 | 8q13.1 | Consensussequence | Hs.29131.0 | AW027474 | Hs.29131.0 |
| 51 | 230500_at | PDE7A | phosphodiesterase 7A | 8q13 | Consensussequence | Hs.150395.2 | AA651933 | Hs.150395.2_RC |
| 52 | 208730_x_at | RAB2 | RAB2, member RAS oncogene family | 8q12.1 | Consensussequence | Hs.78305.0 | NM_002865.1 | Hs.78305.0_RC |
| 53 | 219993_at | SOX17 | SRY (sex determining region Y)-box 17 | 8q11.22 | Exemplarsequence | Hs.97984.0 | NM_022454.1 | g11967990 |
| 54 | 204837_at | MTMR9 | myotubularin related protein 9 | 8p23-p22 | Consensussequence | Hs.27194.0 | AL080178.1 | Hs.27194.0.S2 |
| 55 | 228615_at | LOC286161 | hypothetical protein LOC286161 | 8p23.3 | Consensussequence | Hs.13477.0 | AW291761 | Hs.13477.0_RC |
| 56 | 220512_at | FLJ21120 | hypothetical protein FLJ21120 | 8p22 | Exemplarsequence | Hs.133546.0 | NM_024767.1 | g13376110 |
| 57 | 203548_s_at | LPL | lipoprotein lipase | 8p22 | Consensussequence | Hs.180878.0 | BF672975 | Hs.180878.0_RC |
| 58 | 202680_at | GTF2E2 | general transcription factor IIE, | 8p21-p12 | Exemplarsequence | Hs.77100.0 | NM_002095.1 | g4504194 |
| | | | polypeptide 2, beta 34 kDa | | | | | |
| 59 | 208791_at | CLU | clusterin (complement lysis inhibitor, SP- | 8p21-p12 | Exemplarsequence | Hs.75106.0 | M25915.1 | g180619 |
| | | | 40,40, sulfated glycoprotein 2, | | | | | |
| | | | testosterone-repressed prostate message | | | | | |
| | | | 2, apolipoprotein J) | | | | | |
| 60 | 208792_at | CLU | clusterin (complement lysis inhibitor, SP- | 8p21-p12 | Exemplarsequence | Hs.75106.0 | M25915.1 | g180619 |
| | | | 40,40, sulfated glycoprotein 2, | | | | | |
| | | | testosterone-repressed prostate message | | | | | |
| | | | 2, apolipoprotein J) | | | | | |
| 61 | 208241_at | NRG1 | neuregulin 1 | 8p21-p12 | Exemplarsequence | Hs.172816.4 | NM_004495.1 | g4758525 |
| 62 | 228013_at | | Homo sapiens mRNA; cDNA | | Consensussequence | Hs.61696.0 | AV702575 | Hs.61696.0_RC |
| | | | DKFZp586F1523 (from clone | | | | | |
| | | | DKFZp586F1523) | | | | | |
| 63 | 221236_s_at | STMN4 | stathmin-like 4 | 8p21.1 | Exemplarsequence | g13540510 | NM_030795.1 | g13540510 |
| 64 | 202313_at | PPP2R2A | protein phosphatase 2 (formerly 2A), | 8p21.1 | Exemplarsequence | Hs.179574.0 | NM_002717.1 | g4506018 |
| | | | regulatory subunit B (PR 52), alpha isoform | | | | | |
| 65 | 202035_s_at | SFRP1 | secreted frizzled-related protein 1 | 8p12-p11.1 | Consensussequence | Hs.7306.0 | AI332407 | Hs.7306.0.A1 |
| 66 | 211535_s_at | FGFR1 | fibroblast growth factor receptor 1 (fms- | 8p11.2-p11.1 | Exemplarsequence | Hs.748.6 | M60485.1 | g182560 |
| | | | related tyrosine kinase 2. Pfeiffer syndrome) | | | | | |
| 67 | 207822_at | FGFR1 | fibroblast growth factor receptor 1 (fms- | 8p11.2-p11.1 | Exemplarsequence | Hs.748.0 | NM_023107.1 | g13186237 |
| | | | related tyrosine kinase 2, Pfeiffer syndrome) | | | | | |
| 68 | 236192_at | | ESTs | | Consensussequence | Hs.124961.0 | BF447112 | Hs.124961.0_RC |
| 69 | 207568_at | CHRNA6 | cholinergic receptor, nicotinic, alpha | 8p11.1 | Exemplarsequence | Hs.103128.0 | NM_004198.1 | g4757981 |
| | | | polypeptide 6 | | | | | |
| 70 | 218173_s_at | WHSC1L1 | Wolf-Hirschhorn syndrome candidate 1- | 8p11.2 | Exemplarsequence | Hs.27721.0 | NM_017778.2 | g13699812 |
| | | | like 1 | | | | | |
| 71 | 217437_s_at | TACC1 | transforming, acidic coiled-coil containing | 8p11 | Consensussequence | Hs.173159.1 | AB029026.1 | Hs.173159.1.S1 |
| | | | protein 1 | | | | | |
| 72 | 38158_at | ESPL1 | extra spindle poles like 1 (S. cerevisiae) | 8 | Consensussequence | 4852842_rc | D79987 | 4852842_rc |

| # | Sequence Source | Unigene_Accession | Cluster_Type | LocusLink | Full_Length_Reference_Seq |
|---|---|---|---|---|---|
| 1 | GenBank | Hs.173094 | fulllength | 85453 | NM_033512; KIAA1750 protein |
| 2 | GenBank | Hs.4147 | fulllength | 23471 | NM_014294; translocating chain-associating membrane protein |
| 3 | GenBank | Hs.2463 | fulllength | 284 | NM_001146; angiopoietin 1 isoform a NM_139290; angiopoietin 1 isoform b |
| 4 | RefSeq | Hs.2463 | fulllength | 284 | NM_001146; angiopoietin 1 isoform a NM_139290; angiopoietin 1 isoform b |
| 5 | RefSeq | Hs.273344 | fulllength | 25879 | NM_014156; NM_015420; DKFZP564O0463 protein |
| 6 | GenBank | Hs.98471 | est | 137682 | NM_152416; hypothetical protein MGC40214 |
| 7 | RefSeq | Hs.170198 | fulllength | 9650 | NM_014637; KIAA0009 gene product |
| 8 | GenBank | Hs.131055 | | 286056 | |
| 9 | RefSeq | Hs.425311 | fulllength | 27257 | NM_014462; Lsm1 protein |
| 10 | GenBank | Hs.28285 | fulllength | 11236 | NM_007218; ring finger protein 139 |
| 11 | RefSeq | Hs.169395 | fulllength | 79870 | NM_024812; brain and acute leukemia, cytoplasmic |
| 12 | RefSeq | Hs.241384 | fulllength | 10646 | NM_006550; fibrinogen silencer binding protein |
| 13 | GenBank | Hs.51233 | fulllength | 8795 | NM_003842; tumor necrosis factor receptor superfamily, member 10b isoform 1 precursor NM_147187; tumor necrosis factor |
| | | | | | receptor superfamily, member 10b isoform 2 precursor |
| 14 | GenBank | Hs.7946 | fulllength | 57509 | NM_020749; transcription factor MTSG1 |
| 15 | GenBank | Hs.25590 | fulllength | 6781 | NM_003155; stanniocalcin 1 |
| 16 | RefSeq | Hs.174030 | fulllength | 10863 | NM_014265; a disintegrin and metalloproteinase domain 28 isoform 1 |
| | | | | | preproprotein NM_021777; a disintegrin and metalloproteinase domain |
| | | | | | 28 isoform 3 preproprotein NM_021778; a disintegrin and |
| | | | | | metalloproteinase domain 28 isoform 2 preproprotein |
| 17 | RefSeq | Hs.27410 | fulllength | 55893 | NM_018660; papillomavirus regulatory factor PRF-1 |
| 18 | RefSeq | Hs.153479 | fulllength | 9700 | NM_012291; extra spindle poles like 1 |
| 19 | GenBank | Hs.26102 | fulllength | 7227 | NM_014112; zinc finger transcription factor TRPS1 |
| 20 | RefSeq | Hs.8358 | fulllength | 55638 | NM_017786; hypothetical protein FLJ20366 |
| 21 | GenBank | Hs.55043 | | | |
| 22 | GenBank | Hs.68090 | fulllength | 55909 | NM_018688; bridging integrator 3 |
| 23 | GenBank | Hs.27410 | fulllength | 55893 | NM_018660; papillomavirus regulatory factor PRF-1 |
| 24 | GenBank | Hs.27410 | fulllength | 55893 | NM_018660; papillomavirus regulatory factor PRF-1 |
| 25 | GenBank | Hs.403836 | fulllength | 7373 | |
| 26 | GenBank | Hs.16292 | est | | |
| 27 | GenBank | Hs.170198 | fulllength | 9650 | NM_014637; KIAA009 gene product |
| 28 | RefSeq | Hs.170132 | fulllength | 79873 | NM_024815; hypothetical protein FLJ22494 |
| 29 | GenBank | Hs.72071 | fulllength | 54793 | NM_017634; hypothetical protein FLJ20038 |
| 30 | GenBank | Hs.279704 | fulllength | 54108 | NM_017444; chromatin accessibility complex 1 |
| 31 | RefSeq | Hs.300224 | fulllength | 51236 | NM_016458; brain protein 16 |
| 32 | GenBank | Hs.333388 | fulllength | 93100 | NM_145201; similar to CG3714 gene product |
| 33 | GenBank | Hs.67776 | | | |
| 34 | GenBank | Hs.84087 | | 23167 | |
| 35 | GenBank | Hs.84087 | | 23167 | |
| 36 | GenBank | Hs.26102 | fulllength | 7227 | NM_014112; zinc finger transcription factor TRPS1 |
| 37 | GenBank | Hs.1501 | fulllength | 6383 | NM_002998; syndecan 2 |
| 38 | GenBank | Hs.159274 | fulllength | 4956 | NM_024410; outer dense fiber of sperm tails 1 |
| 39 | RefSeq | Hs.158836 | fulllength | 54990 | NM_017890; hypothetical protein FLJ20583 |
| 40 | GenBank | Ns.243901 | fulllength | | |
| 41 | GenBank | Hs.130554 | fulllength | 91147 | NM_153704; hypothetical protein MGC26979 |
| 42 | RefSeq | Hs.31551 | fulllength | 862 | NM_004349; acute myelogenous leukemia 1 translocation 1 protein isoform MTG8a NM_175634; acute myelogenous leukemia 1 |
| | | | | | translocation 1 protein isoform MTG8b NM_175635; acute myelogenous leukemia 1 translocation 1 protein isoform MTG8c |
| | | | | | NM_175636; acute myelogenous leukemia 1 translocation 1 protein isoform MTG8c |
| 43 | GenBank | Hs.334807 | fulllength | 6156 | NM_000989; ribosomal protein L30 |
| 44 | RefSeq | Hs.128645 | fulllength | 64089 | NM_022133; sorting nexin 16 isoform a NM_152836; sorting nexin 16 isoform a NM_152837; sorting nexin 16 isoform b |
| 45 | GenBank | Hs.266175 | fulllength | 55824 | NM_018440; phosphoprotein associated with glycosphingolipid-enriched |
| | | | | | microdomains |
| 46 | GenBank | Hs.40539 | fulllength | 734 | NM_004337; chromosome 8 open reading frame 1 |
| 47 | GenBank | Hs.234434 | fulllength | 23462 | NM_012258; hairy/enhancer-of-split related with YRPW motif 1 |
| 48 | GenBank | Hs.23118 | fulllength | 759 | NM_001738; carbonic anhydrase I |
| 49 | RefSeq | Hs.184693 | fulllength | 6921 | NM_005648; elongin C |
| 50 | GenBank | Hs.404048 | fulllength | 10499 | NM_006540; nuclear receptor coactivator 2 |
| 51 | GenBank | Hs.406325 | fulllength | 5150 | NM_002603; phosphodiesterase 7A isoform a NM_002604; |
| | | | | | phosphodiesterase 7A isoform b |
| 52 | GenBank | Hs.78305 | fulllength | 5862 | NM_002865; RAB2, member RAS oncogene family |
| 53 | RefSeq | Hs.97984 | fulllength | 64321 | NM_022454; SRY-box 17 |
| 54 | GenBank | Hs.48802 | fulllength | 66036 | NM_015458; myotubularin related protein 9 |
| 55 | GenBank | Hs.13477 | | 286161 | |
| 56 | RefSeq | Hs.133546 | fulllength | 79824 | |
| 57 | GenBank | Hs.180878 | fulllength | 4023 | NM_000237; lipoprotein lipase precursor |
| 58 | RefSeq | Hs.77100 | fulllength | 2961 | NM_002095; general transcription factor IIE, polypeptide 2, beta 34 kDa |
| 59 | GenBank | Hs.75106 | fulllength | 1191 | NM_001831; clusterin |
| 60 | GenBank | Hs.75106 | fulllength | 1191 | NM_001831; clusterin |
| 61 | RefSeq | Hs.172816 | fulllength | 3084 | NM_004495; neuregulin 1 isoform HRG-gamma NM_013956; neuregulin |
| | | | | | 1 isoform HRG-beta1 NM_013957; neuregulin 1 isoform HRG-beta2 |
| | | | | | NM_013958; neuregulin 1 isoform HRG-beta3 NM_013959; neuregulin 1 |
| | | | | | isoform SMDF NM_013960; neuregulin 1 isoform ndf43 NM_013961; |
| | | | | | neuregulin 1 isoform GGF NM_013962; nauregulin 1 isoform GGF2 |
| | | | | | NM_013964; neuregulin 1 isoform HRG-alpha |
| 62 | GenBank | Hs.61696 | | | |
| 63 | RefSeq | Hs.3815 | fulllength | 81551 | NM_030795; stathmin-like-protein RB3 |
| 64 | RefSeq | Hs.179574 | fulllength | 5520 | NM_002717; protein phosphatase 2 (formerly 2A), regulatory subunit B |
| | | | | | (PR 52), alpha isoform |
| 65 | GenBank | Hs.7306 | fulllength | 6422 | NM_003012; secreted frizzled-related protein 1 |
| 66 | GenBank | Hs.748 | fulllength | 2260 | NM_000604; fibroblast growth factor receptor 1 isoform 1 precursor |
| | | | | | NM_015850; fibroblast growth factor receptor 1 isoform 2 precursor |
| | | | | | NM_023105; fibroblast growth factor receptor 1 isoform 3 precursor |
| | | | | | NM_023106; fibroblast growth factor receptor 1 isoform 4 precursor |
| | | | | | NM_023107; fibroblast growth factor receptor 1 isoform 5 precursor |
| | | | | | NM_023108; fibroblast growth factor receptor 1 isoform 6 precursor |
| | | | | | NM_023109; fibroblast growth factor receptor 1 isoform 7 precursor |
| | | | | | NM_023110; fibroblast growth factor receptor 1 isoform 8 precursor |
| | | | | | NM_023111; fibroblast growth factor receptor 1 isoform 9 precursor |
| 67 | RefSeq | Hs.748 | fulllength | 2260 | NM_000604; fibroblast growth factor receptor 1 isoform 1 precursor |
| | | | | | NM_015850; fibroblast growth factor receptor 1 isoform 2 precursor |
| | | | | | NM_023105; fibroblast growth factor receptor 1 isoform 3 precursor |
| | | | | | NM_023106; fibroblast growth factor receptor 1 isoform 4 precursor |
| | | | | | NM_023107; fibroblast growth factor receptor 1 isoform 5 precursor |
| | | | | | NM_023108; fibroblast growth factor receptor 1 isoform 6 precursor |
| | | | | | NM_023109; fibroblast growth factor receptor 1 isoform 7 precursor |
| | | | | | NM_023110; fibroblast growth factor receptor 1 isoform 8 precursor |
| | | | | | NM_023111; fibroblast growth factor receptor 1 isoform 9 precursor |
| 68 | GenBank | Hs.124961 | est | | |
| 69 | RefSeq | Hs.103128 | fulllength | 8973 | NM_004198; cholinergic receptor, nicotinic, alpha polypeptide 6 |
| 70 | RefSeq | Hs.27721 | fulllength | 54904 | NM_017778; WHSC1L1 protein isoform short NM_023034; WHSC1L1 |
| | | | | | protein isoform long |
| 71 | GenBank | Hs.173159 | fulllength | 6867 | NM_006283; transforming, acidic coiled-coil containing protein 1 |
| 72 | GenBank | Hs.153479 | fulllength | 9700 | NM_012291; extra spindle poles like 1 |

TABLE 29
_normal

| | | HUGO | | | | | | | | | |
|---|---|---|---|---|---|---|---|---|---|---|---|
| # | affy id | name | fc | p | q | stn | t | Title | MapLocation | Sequence Type | Transcript ID |
| 1 | 200923_at | LGALS3BP | −6.84 | 6.78e−23 | 2.44e−18 | −0.75 | −11.10 | lectin, galactoside-binding, soluble, 3 binding | 17q25 | Exemplarsequence | Hs.79339.0 |
| | | | | | | | | protein | | | |
| 2 | 212250_at | | 17533 | 1.95e−09 | 6.40e−06 | 1.00 | 47362 | Homo sapiens, Similar to LYRIC, clone | | Consensussequence | Hs.243901.0 |
| | | | | | | | | MGC: 41931 IMAGE: 5298467, mRNA, complete | | | |
| | | | | | | | | cds | | | |
| 3 | 206761_at | TACTILE | −5.48 | 7.94e−15 | 1.43e−10 | −0.59 | −8.53 | T cell activation, increased late expression | 3q13.13 | Exemplarsequence | Hs.142023.0 |
| 4 | 213110_s_at | COL4A5 | −3.52 | 4.83e−12 | 3.48e−08 | −0.63 | −8.28 | collagen, type IV, alpha 5 (Alport | Xq22 | Consensussequence | Hs.169825.0 |
| | | | | | | | | syndrome) | | | |
| 5 | 203110_at | PTK2B | 27030 | 2.85e−08 | 6.04e−05 | 0.90 | 41852 | PTK2B protein tyrosine kinase 2 beta | 8p21.1 | Exemplarsequence | Hs.20313.0 |
| 6 | 225233_at | | −3.41 | 4.70e−13 | 5.64e−09 | −0.54 | −7.87 | Homo sapiens, clone IMAGE: 4154313, | | Consensussequence | Hs.42179.0 |
| | | | | | | | | mRNA, partial cds | | | |
| 7 | 205910_s_at | CEL | −4.12 | 6.40e−13 | 5.77e−09 | −0.52 | −7.67 | carboxyl ester lipase (bile salt-stimulated | 9q34.3 | Exemplarsequence | Hs.99918.0 |
| | | | | | | | | lipase) | | | |
| 8 | 225240_s_at | | −2.84 | 6.34e−10 | 2.86e−06 | −0.62 | −7.64 | Homo sapiens, clone IMAGE: 4154313, | | Consensussequence | Hs.42179.0 |
| | | | | | | | | mRNA, partial cds | | | |
| 9 | 216412_x_at | IGL | −2.42 | 3.13e−09 | 8.67e−06 | −0.53 | −6.87 | immunoglobulin lambda locus | 22q11.1-q11.2 | Consensussequence | Hs.287815.0 |
| 10 | 209050_s_at | RALGDS | −1.60 | 3.12e−10 | 1.61e−06 | −0.48 | −6.82 | ral guanine nucleotide dissociation | 9q34.3 | Consensussequence | Hs.106185.0 |
| | | | | | | | | stimulator | | | |
| 11 | 227943_at | | −1.93 | 2.75e−09 | 8.26e−06 | −0.49 | −6.65 | ESTs | | Consensussequence | Hs.25933.0 |
| 12 | 226282_at | | −8.88 | 2.44e−10 | 1.46e−06 | −0.45 | −6.65 | Homo sapiens cDNA FLJ32401 fis, clone | | Consensussequence | Hs.235857.0 |
| | | | | | | | | SKMUS2000339. | | | |
| 13 | 219553_at | NME7 | −1.60 | 1.17e−09 | 4.22e−06 | −0.47 | −6.63 | non-metastatic cells 7, protein expressed | 1q24 | Exemplarsequence | Hs.274479.0 |
| | | | | | | | | in (nucleoside-diphosphate kinase) | | | |
| 14 | 236124_at | LOC153546 | −1.64 | 3.75e−08 | 7.12e−05 | −0.54 | −6.61 | hypothetical protein LOC153546 | 5p13.3 | Consensussequence | Hs.44104.0 |
| 15 | 240929_at | | 14977 | 7.28e−07 | 5.70e−04 | 0.64 | 15128 | Homo sapiens cDNA FLJ25206 fis, clone | | Consensussequence | Hs.126813.0 |
| | | | | | | | | REC05955. | | | |
| 16 | 221286_s_at | PACAP | −5.90 | 9.01e−10 | 3.61e−06 | −0.43 | −6.41 | proapoptotic caspase adaptor protein | 5q23-5q31 | Exemplarsequence | Hs.122492.1 |
| 17 | 225237_s_at | | −2.41 | 2.14e−07 | 2.57e−04 | −0.56 | −6.37 | Homo sapiens, clone IMAGE: 4154313, | | Consensussequence | Hs.42179.0 |
| | | | | | | | | mRNA, partial cds | | | |
| 18 | 214029_at | | −2.36 | 1.64e−08 | 3.70e−05 | −0.47 | −6.30 | Homo sapiens, clone MGC: 26694 | | Consensussequence | Hs.20707.0 |
| | | | | | | | | IMAGE: 4819096, mRNA, complete cds | | | |
| 19 | 209014_at | MAGED1 | −1.65 | 8.44e−08 | 1.38e−04 | −0.49 | −6.17 | melanoma antigen, family D, 1 | Xp11.23 | Exemplarsequence | Hs.177556.0 |
| 20 | 213185_at | KIAA0556 | −1.27 | 5.02e−07 | 4.86e−04 | −0.53 | −6.08 | KIAA0556 protein | 16p12.1 | Consensussequence | Hs.30512.0 |
| 21 | 200974_at | ACTA2 | −1.72 | 1.89e−07 | 2.35e−04 | −0.49 | −6.06 | actin, alpha 2, smooth muscle, aorta | 10q23.3 | Exemplarsequence | Hs.195851.0 |
| 22 | 228539_at | | −2.56 | 4.38e−07 | 4.51e−04 | −0.50 | −5.98 | ESTs | | Consensussequence | Hs.296100.0 |
| 23 | 228598_at | DPRP3 | −3.05 | 1.08e−08 | 2.78e−05 | −0.40 | −5.96 | dipeptidyl peptidase IV-retated protein 3 | 2q13 | Consensussequence | Hs.91625.0 |
| 24 | 223251_s_at | ANKRD10 | −1.58 | 1.71e−06 | 1.01e−03 | −0.57 | −5.96 | ankyrin repeat domain 10 | 13q33.3 | Exemplarsequence | Hs.298998.0 |
| 25 | 209051_s_at | RALGDS | −1.73 | 1.77e−07 | 2.28e−04 | −0.47 | −5.96 | ral guanine nucleotide dissociation | 9q34.3 | Exemplarsequence | Hs.106185.0 |
| | | | | | | | | stimulator | | | |
| 26 | 225238_at | | −2.85 | 3.54e−07 | 3.98e−04 | −0.49 | −5.95 | Homo sapiens, clone IMAGE: 4154313, | | Consensussequence | Hs.42179.0 |
| | | | | | | | | mRNA, partial cds | | | |
| 27 | 205801_s_at | GRP3 | −3.12 | 1.17e−07 | 1.66e−04 | −0.45 | −5.94 | guanine nucleotide exchange factor for | 2p25.1-p24.1 | Exemplarsequence | Hs.24024.0 |
| | | | | | | | | Rap1 | | | |
| 28 | 202365_at | MGC5139 | −1.48 | 5.14e−07 | 4.86e−04 | −0.50 | −5.92 | hypothetical protein MGC5139 | 12q24.31 | Exemplarsequence | Hs.127610.0 |
| 29 | 244533_at | | −3.90 | 1.30e−08 | 3.12e−05 | −0.40 | −5.91 | ESTs, Weakly similar to hypothetical | | Consensussequence | Hs.294079.0 |
| | | | | | | | | protein FLJ20489 [Homo sapiens] | | | |
| | | | | | | | | [H. sapiens] | | | |
| 30 | 226134_s_at | | −2.74 | 1.52e−06 | 9.78e−04 | −0.54 | −5.89 | Homo sapiens, clone IMAGE: 4154313, | | Consensussequence | Hs.42640.0 |
| | | | | | | | | mRNA, partial cds | | | |
| 31 | 222742_s_at | FLJ14117 | −1.64 | 2.41e−06 | 1.31e−03 | −0.57 | −5.89 | hypothetical protein FLJ14117 | 7q22.1 | Consensussequence | Hs.61809.0 |
| 32 | 206506_s_at | SUPT3H | −1.60 | 1.05e−06 | 7.57e−04 | −0.52 | −5.89 | suppressor of Ty 3 homolog (S. cerevisiae) | 6p21.1-p21.3 | Exemplarsequence | Hs.96757.0 |
| 33 | 229949_at | | −1.65 | 3.09e−07 | 3.59e−04 | −0.46 | −5.85 | Homo sapiens CDNA FLJ33372 fis, clone | | Consensussequence | Hs.325158.0 |
| | | | | | | | | BRACE2005981. | | | |
| 34 | 201808_s_at | ENG | −1.75 | 8.82e−07 | 6.62e−04 | −0.50 | −5.82 | endoglin (Osler-Rendu-Weber syndrome | 9q33-q34.1 | Consensussequence | Hs.76753.0 |
| | | | | | | | | 1) | | | |
| 35 | 225314_at | MGC45416 | −1.67 | 3.27e−06 | 1.65e−03 | −0.58 | −5.82 | hypothetical protein MGC45416 | 4p11 | Consensussequence | Hs.95835.0 |
| 36 | 219602_s_at | FLJ23403 | −1.80 | 6.94e−08 | 1.19e−04 | −0.42 | −5.81 | hypothetical protein FLJ23403 | 18p11.21 | Exemplarsequence | Hs.293907.0 |
| 37 | 219218_at | FLJ23058 | −1.99 | 6.67e−07 | 5.70e−04 | −0.48 | −5.78 | hypothetical protein FLJ23058 | 17q25.3 | Exemplarsequence | Hs.98968.0 |
| 38 | 203007_x_at | LYPLA1 | 18629 | 1.09e−05 | 4.02e−03 | 0.74 | 28246 | lysophospholipase I | 8q11.23 | Exemplarsequence | Hs.12540.0 |
| 39 | 201664_at | SMC4L1 | −1.48 | 3.64e−07 | 3.98e−04 | −0.45 | −5.77 | SMC4 structural maintenance of | 3q26.1 | Exemplarsequence | Hs.50758.0 |
| | | | | | | | | chromosomes 4-like 1 (yeast) | | | |
| 40 | 206847_s_at | HOXA7 | −1.86 | 2.36e−06 | 1.31e−03 | −0.53 | −5.76 | homeo box A7 | 7p15-p14 | Exemplarsequence | Hs.70954.0 |
| 41 | 221525_at | DKFZp761I2123 | −1.62 | 9.80e−08 | 1.53e−04 | −0.42 | −5.76 | hypothetical protein DKFZp761I2123 | 7p12.3 | Exemplarsequence | Hs.77978.0 |
| 42 | 227725_at | ST6GalNAcI | −6.39 | 3.12e−08 | 6.24e−05 | −0.40 | −5.76 | GalNAc alpha-2, 6-sialyltransferase I, long | 17q25.3 | Consensussequence | Hs.105352.0 |
| | | | | | | | | form | | | |
| 43 | 239237_at | | −1.89 | 1.13e−06 | 7.96e−04 | −0.49 | −5.75 | ESTs | | Consensussequence | Hs.265130.0 |
| 44 | 243579_at | MSI2 | −2.96 | 1.90e−06 | 1.10e−03 | −0.51 | −5.72 | musashi homolog 2 (Drosophila) | 17q23.1 | Consensussequence | Hs.173179.0 |
| 45 | 230974_at | DDX19 | −2.27 | 5.13e−08 | 9.24e−05 | −0.39 | −5.71 | DEAD/H (Asp-Glu-Ala-Asp/His) box | 16q22.1 | Consensussequence | Hs.127325.0 |
| | | | | | | | | polypeptide 19 (DBP5 homolog, yeast) | | | |
| 46 | 200608_s_at | RAD21 | 14611 | 1.21e−05 | 4.22e−03 | 0.72 | 25689 | RAD21 homolog (S. pombe) | 8q24 | Exemplarsequence | Hs.81848.0 |
| 47 | 237291_at | | −1.78 | 2.71e−06 | 1.44e−03 | −0.53 | −5.70 | ESTs, Weakly similar to unknown protein | | Consensussequence | Hs.159362.0 |
| | | | | | | | | [Arabidopsis thaliana] [A. thaliana] | | | |
| 48 | 209365_s_at | ECM1 | −1.60 | 7.18e−07 | 5.70e−04 | −0.46 | −5.69 | extracellular matrix protein 1 | 1q21 | Exemplarsequence | Hs.81071.1 |
| 49 | 235521_at | HOXA3 | −2.52 | 1.95e−06 | 1.11e−03 | −0.50 | −5.67 | homeo box A3 | 7p15-p14 | Consensussequence | Hs.222446.0 |
| 50 | 201433_s_at | PTDSS1 | 22647 | 1.58e−05 | 5.13e−03 | 0.78 | 24593 | phosphatidylserine synthase 1 | 8q22 | Exemplarsequence | Hs.77329.0 |

| | Sequence | | Sequence | | | | |
|---|---|---|---|---|---|---|---|
| # | Derived From | Sequence ID | Source | Unigene_Accession | Cluster_Type | LocusLink | Full_Length_Reference_Seq |
| 1 | NM_005567.2 | g6006016 | RefSeq | Hs.79339 | fulllength | 3959 | NM_005567; galectin 3 binding protein |
| 2 | AI972475 | Hs.243901.0.S1 | GenBank | Hs.243901 | fulllength | | |
| 3 | NM_005816.1 | g5032140 | RefSeq | Hs.142023 | fulllength | 10225 | NM_005816; T cell activation, increased late expression |
| 4 | AW052179 | Hs.169825.0_RC | GenBank | Hs.169825 | fulllength | 1287 | NM_000495; alpha 5 type IV collagen isoform 1, precursor NM_033380; |
| | | | | | | | alpha 5 type IV collagen isoform 2, precursor NM_033381; alpha 5 type IV |
| | | | | | | | collagen isoform 3, precursor |
| 5 | U43522.1 | g1165218 | GenBank | Hs.20313 | fulllength | 2185 | NM_004103; PTK2B protein tyrosine kinase 2 beta isoform a NM_173174; |
| | | | | | | | PTK2B protein tyrosine kinase 2 beta isoform a NM_173175; PTK2B protein |
| | | | | | | | tyrosine kinase 2 beta isoform b NM_173176; PTK2B protein tyrosine kinase 2 beta |
| | | | | | | | isoform a |
| 6 | BF435123 | Hs.42179.0.A1 | GenBank | Hs.173179 | | | |
| 7 | NM_001807.1 | g4502770 | RefSeq | Hs.406160 | fulllength | 1056 | NM_001807; carboxyl ester lipase precursor |
| 8 | BF435123 | Hs.42179.0.A1 | GenBank | Hs.173179 | | | |
| 9 | AF043584.1 | Hs.287815.0 | GenBank | Hs.405944 | fulllength | 3535 | NG_000002; |
| 10 | AI421559 | Hs.106185.0 | GenBank | Hs.106185 | fulllength | 5900 | NM_006266; ral guanine nucleotide dissociation stimulator |
| 11 | AI798680 | Hs.25933.0_RC | GenBank | Hs.445018 | est | | |
| 12 | AW129783 | Hs.235857.0_RC | GenBank | Hs.235857 | | | |
| 13 | NM_013330.2 | g7242158 | RefSeq | Hs.274479 | fulllength | 29922 | NM_013330; NME7 |
| 14 | AL037070 | Hs.44104.0_RC | GenBank | Hs.422878 | | 153546 | |
| 15 | AW300004 | Hs.126813.0_RC | GenBank | Hs.126813 | | | |
| 16 | NM_016459.1 | g7706002 | RefSeq | Hs.409563 | fulllength | 51237 | NM_016459; proapoptotic caspase adaptor protein |
| 17 | BF435123 | Hs.42179.0.A1 | GenBank | Hs.173179 | | | |
| 18 | AI435954 | Hs.20707.0.A1 | GenBank | Hs.20707 | fulllength | | |
| 19 | AF217963.1 | g9963809 | GenBank | Hs.5258 | fulllength | 9500 | NM_006986; melanoma antigen, family D, 1 |
| 20 | AI758896 | Hs.30512.0 | GenBank | Hs.30512 | | 23247 | |
| 21 | NM_001613.1 | g4501882 | RefSeq | Hs.195851 | fulllength | 59 | NM_001613; alpha 2 actin |
| 22 | AI097095 | Hs.296100.0_RC | GenBank | Hs.296100 | est | | |
| 23 | AL538781 | Hs.91625.0.S1 | GenBank | Hs.91625 | fulllength | 57628 | NM_020868; dipeptidyl peptidase IV-related protein 3 |
| 24 | BC001727.1 | g12804610 | GenBank | Hs.172572 | fulllength | 55608 | NM_017664; ankyrin repeat domain 10 |
| 25 | AF295773.1 | g9931301 | GenBank | Hs.106185 | fulllength | 5900 | NM_006266; ral guanine nucleotide dissociation stimulator |
| 26 | BF435123 | Hs.42179.0.A1 | GenBank | Hs.173179 | | | |
| 27 | NM_015376.1 | g7662333 | RefSeq | Hs.24024 | fulllength | 25780 | NM_015376; RAS guanyl releasing protein 3 (calcium and DAG-regulated) |
| | | | | | | | NM_170672; RAS guanyl releasing protein 3 (calcium and DAG-regulated) |
| 28 | BC004815.1 | g13435956 | GenBank | Hs.127610 | fulllength | 84747 | NM_032661; hypothetical protein MGC5139 |
| 29 | BE617483 | Hs.294079.0_RC | GenBank | Hs.294079 | est | | |
| 30 | AI978754 | Hs.42640.0.A1 | GenBank | Hs.173179 | | | |
| 31 | AW026449 | Hs.61809.0 | GenBank | Hs.61809 | fulllength | 64792 | NM_022777; hypothetical protein FLJ14117 |
| 32 | NM_003599.1 | g4507308 | RefSeq | Hs.304173 | fulllength | 8464 | NM_003599; suppressor of Ty 3 homolog |
| 33 | AA554827 | Hs.325158.0_RC | GenBank | Hs.370705 | | | |
| 34 | BE732652 | Hs.76753.0 | GenBank | Hs.76753 | fulllength | 2022 | NM_000118; endoglin precursor |
| 35 | BG291649 | Hs.95835.0.A1 | GenBank | Hs.95835 | fulllength | 132299 | NM_152398; hypothetical protein MGC45416 |
| 36 | NM_022068.1 | g13384601 | RefSeq | Hs.293907 | fulllength | 63895 | NM_022068; hypothetical protein FLJ23403 |
| 37 | NM_024696.1 | g13375978 | RefSeq | Hs.98968 | fulllength | 79749 | NM_024696; hypothetical protein FLJ23058 |
| 38 | AF077198.1 | g4679009 | GenBank | Hs.12540 | fulllength | 10434 | NM_006330; lysophospholipase I |
| 39 | AL136877.1 | g6807670 | GenBank | Hs.50758 | fulllength | 10051 | NM_005496; SMC4 structural maintenance of chromosomes 4-like 1 |
| 40 | AF026397.1 | g2739070 | GenBank | Hs.446318 | fulllength | 3204 | NM_006896; homeobox protein A7 |
| 41 | AL136572.1 | g13276646 | GenBank | Hs.77978 | fulllength | 83637 | |
| 42 | Y11339.2 | Hs.105352.0 | GenBank | Hs.105352 | fulllength | 55808 | NM_018414; GalNAc alpha-2, 6-sialyltransferase I |
| 43 | AI798822 | Hs.265130.0_RC | GenBank | Hs.265130 | est | | |
| 44 | BF029215 | Hs.173179.0.S1 | GenBank | Hs.103512 | fulllength | 124540 | NM_138962; musashi 2 isoform a NM_170721; musashi 2 isoform b |
| 45 | AA234116 | Hs.127325.0_RC | GenBank | Hs.289097 | fulllength | 11269 | NM_007242; DEAD/H (Asp-Glu-Ala-Asp/His) box polypeptide 19 |
| 46 | NM_006265.1 | g5453993 | RefSeq | Hs.81848 | fulllength | 5885 | NM_006265; RAD21 homolog |
| 47 | AI695007 | Hs.159362.0.A1 | GenBank | Hs.159362 | est | | |
| 48 | U65932.1 | g1488323 | GenBank | Hs.81071 | fulllength | 1893 | NM_004425; extracellular matrix protein 1 isoform 1 precursor NM_022664; |
| | | | | | | | extracellular matrix protein 1 isoform 2 precursor |
| 49 | AW137982 | Hs.222446.0.A1 | GenBank | Hs.248074 | fulllength | 3200 | NM_030661; homeobox A3 protein isoform a NM_153631; homeobox A3 |
| | | | | | | | protein isoform a NM_153632; homeobox A3 protein isoform b |
| 50 | NM_014754.1 | g7662646 | RefSeq | Hs.77329 | fulllength | 9791 | NM_014754; phosphatidylserine synthase 1 |

TABLE 30
_normal

| | | | | | | | Sequence | | Sequence |
|---|---|---|---|---|---|---|---|---|---|
| # | affy Id | HUGO name | Title | MapLocation | Sequence Type | Transcript ID | Derived From | Sequence ID | Source |
| 1 | 218777_at | FLJ22246 | hypothetical protein FLJ22246 | 8p21.2 | Exemplarsequence | Hs.289063.0 | NM_025232.1 | g13376835 | RefSeq |
| 2 | 225053_at | CNOT7 | CCR4-NOT transcription | 8p22-p21.3 | Consensussequence | Hs.226318.1 | W94952 | Hs.226318.1.A2 | GenBank |
| | | | complex, subunit 7 | | | | | | |
| 3 | 224413_s_at | BLP1 | BBP-like protein 1 | 8p11.21 | Exemplarsequence | g13625460 | AF353991.1 | g13625460 | GenBank |
| 4 | 205849_s_at | UQCRB | ubiquinol-cytochrome c | 8q22 | Exemplarsequence | Hs.131255.0 | NM_006294.1 | g5454151 | RefSeq |
| | | | reductase binding protein | | | | | | |
| 5 | 227001_at | | Homo sapiens cDNA: FLJ21362 | | Consensussequence | Hs.167011.0 | AI096706 | Hs.167011.0 | GenBank |
| | | | fis, clone COL02886. | | | | | | |
| 6 | 212523_s_at | KIAA0146 | KIAA0146 protein | 8q11.21 | Consensussequence | Hs.278634.0 | D63480.1 | Hs.278634.0_RC | GenBank |
| 7 | 219124_at | FLJ23263 | hypothetical protein FLJ23263 | 8p11.23 | Exemplarsequence | Hs.288716.0 | NM_025115.1 | g13376690 | RefSeq |
| 8 | 201398_s_at | TRAM | translocating chain-associating | 8q13.1 | Exemplarsequence | Hs.4147.0 | BC000687.1 | g12653796 | GenBank |
| | | | membrane protein | | | | | | |
| 9 | 235509_at | MGC40214 | hypothetical protein MGC40214 | 8q22.1 | Consensussequence | Hs.98471.0 | AV662196 | Hs.98471.0_RC | GenBank |
| 10 | 203208_s_at | CHPPR | likely ortholog of chicken | 8q12.1 | Exemplarsequence | Hs.170198.0 | NM_014637.1 | g7661853 | RefSeq |
| | | | chondrocyte protein with a poly- | | | | | | |
| | | | proline region | | | | | | |
| 11 | 203207_s_at | CHPPR | likely ortholog of chicken | 8q12.1 | Consensussequence | Hs.170198.0 | BF214329 | Hs.170198.0.S1 | GenBank |
| | | | chondrocyte protein with a poly- | | | | | | |
| | | | proline region | | | | | | |
| 12 | 212248_at | | Homo sapiens, Similar to LYRIC, clone MGC: 41931 | | Consensussequence | Hs.243901.0 | AI972475 | Hs.243901.0.S1 | GenBank |
| | | | IMAGE: 5298467, mRNA, complete cds | | | | | | |
| 13 | 202680_at | GTF2E2 | general transcription factor IIE, | 8p21-p12 | Exemplarsequence | Hs.77100.0 | NM_002095.1 | g4504194 | RefSeq |
| | | | polypeptide 2, beta 34 kDa | | | | | | |
| 14 | 218173_s_at | WHSC1L1 | Wolf-Hirschhorn syndrome | 8p11.2 | Exemplarsequence | Hs.27721.0 | NM_017778.2 | g13699812 | RefSeq |
| | | | candidate 1-like 1 | | | | | | |
| 15 | 218187_s_at | FLJ20989 | hypothetical protein FLJ20989 | | Exemplarsequence | Hs.169615.0 | NM_023080.1 | g12751496 | RefSeq |
| 16 | 208647_at | FDFT1 | farnesyl-diphosphate | 8p23.1-p22 | Consensussequence | Hs.48876.1 | AA872727 | Hs.48876.1 | GenBank |
| | | | farnesyltransferase 1 | | | | | | |
| 17 | 200090_at- | FNTA | farnesyltransferase, CAAX box, | 8p22-q11 | Consensussequence | Hs.138381.1 | BG168896 | Hs.138381.1.A1 | GenBank |
| | HG-U133A | | alpha | | | | | | |
| 18 | 218250_s_at | CNOT7 | CCR4-NOT transcription | 8p22-p21.3 | Exemplarsequence | Hs.226318.0 | NM_013354.2 | g10518495 | RefSeq |
| | | | complex, subunit 7 | | | | | | |
| 19 | 202174_s_at | PCM1 | pericentriolar material 1 | 8p22-p21.3 | Exemplarsequence | Hs.75737.0 | NM_006197.1 | g5453855 | RefSeq |
| 20 | 214118_x_at | PCM1 | pericentriolar material 1 | 8p22-p21.3 | Consensussequence | Hs.75737.1 | AI205598 | Hs.75737.1.S1 | GenBank |
| 21 | 214937_x_at | PCM1 | pericentriolar material 1 | 8p22-p21.3 | Consensussequence | Hs.75737.2 | AI924817 | Hs.75737.2.S1 | GenBank |
| 22 | 222544_s_at | WHSC1L1 | Wolf-Hirschhom syndrome | 8p11.2 | Consensussequence | Hs.27721.0 | AI697751 | Hs.27721.0_RC | GenBank |
| | | | candidate 1-like 1 | | | | | | |
| 23 | 219819_s_at | MRPS28 | mitochondrial ribosomal protein | 8q21.1-q21.2 | Exemplarsequence | Hs.55097.0 | NM_014018.1 | g7661729 | RefSeq |
| | | | S28 | | | | | | |
| 24 | 201618_x_at | GPAA1 | GPAA1P anchor attachment | 8q24.3 | Exemplarsequence | Hs.4742.0 | NM_003801.2 | g6031166 | RefSeq |
| | | | protein 1 homolog (yeast) | | | | | | |
| 25 | 200936_at | RPL8 | ribosomal protein L8 | 8q24.3 | Exemplarsequence | Hs.178551.0 | NM_000973.1 | g4506662 | RefSeq |
| 26 | 236533_at | DDEF1 | development and differentiation | 8q24.1-q24.2 | Consensussequence | Hs.199057.0 | AW236958 | Hs.199057.0.A1 | GenBank |
| | | | enhancing factor 1 | | | | | | |
| 27 | 238562_at | | ESTs, Weakly similar to | | Consensussequence | Hs.280297.0 | BE542779 | Hs.280297.0.A1 | GenBank |
| | | | hypothetical protein FLJ20378 | | | | | | |
| | | | [Homo sapiens] [H. sapiens] | | | | | | |
| 28 | 212250_at | | Homo sapiens, Similar to LYRIC, | | Consensussequence | Hs.243901.0 | AI972475 | Hs.243901.0.S1 | GenBank |
| | | | clone MGC: 41931 | | | | | | |
| | | | IMAGE: 5298467, mRNA, | | | | | | |
| | | | complete cds | | | | | | |
| 29 | 227277_at | | Homo sapiens cDNA FLJ40968 | | Consensussequence | Hs.33074.0 | BG530089 | Hs.33074.0.S1 | GenBank |
| | | | fis, clone UTERU2012615. | | | | | | |
| 30 | 202119_s_at | CPNE3 | copine III | 8q21.13 | Exemplarsequence | Hs.14158.0 | NM_003909.1 | g4503014 | RefSeq |
| 31 | 218919_at | FLJ14007 | hypothetical protein FLJ14007 | 8q21.12 | Exemplarsequence | Hs.99519.0 | NM_024699.1 | g13375984 | RefSeq |
| 32 | 202955_s_at | BIG1 | brefeldin A-inhibited guanine | 8q13 | Exemplarsequence | Hs.94631.0 | AF084520.1 | g5052120 | GenBank |
| | | | nucleotide-exchange protein 1 | | | | | | |
| 33 | 203448_s_at | TERF1 | telomeric repeat binding factor | 8q13 | Consensussequence | Hs.194562.0 | AI347136 | Hs.194562.0.A1 | GenBank |
| | | | (NIMA-interacting) 1 | | | | | | |
| 34 | 204301_at | KIAA0711 | KIAA0711 gene product | 8p23.2 | Exemplarsequence | Hs.5333.0 | NM_014867.1 | g7662259 | RefSeq |
| 35 | 210980_s_at | ASAH1 | N-acylsphingosine | 8p22-p21.3 | Exemplarsequence | Hs.75811.1 | U47674.1 | g3860239 | GenBank |
| | | | amidohydrolase (acid | | | | | | |
| | | | ceramidase) 1 | | | | | | |
| 36 | 208231_at | NRG1 | neuregulin 1 | 8p21-p12 | Exemplarsequence | Hs.172816.0 | NM_013960.1 | g7669519 | RefSeq |
| 37 | 205770_at | GSR | glutathione reductase | 8p21.1 | Exemplarsequence | Hs.121524.0 | NM_000637.1 | g10835188 | RefSeq |
| 38 | 217819_at | LOC51125 | HSPC041 protein | 8p11.21 | Exemplarsequence | Hs.7953.0 | NM_016099.1 | g7705820 | RefSeq |
| 39 | 209517_s_at | ASH2L | ash2 (absent, small, or | 8p11.2 | Exemplarsequence | Hs.6856.1 | AB020982.1 | g4417209 | GenBank |
| | | | homeotic)-like (Drosophila) | | | | | | |
| 40 | 222525_s_at | FLJ10853 | hypothetical protein FLJ10853 | 8p21.1 | Consensussequence | Hs.72085.0 | AU160632 | Hs.72085.0 | GenBank |
| 41 | 222998_at | MAF1 | homolog of yeast MAF1 | 8q24.3 | Exemplarsequence | Hs.324157.0 | AL136937.1 | g12053368 | GenBank |
| 42 | 218679_s_at | VPS28 | vacuolar protein sorting 28 | 8q24.3 | Exemplarsequence | Hs.293441.0 | NM_016208.1 | g7705884 | RefSeq |
| | | | (yeast) | | | | | | |
| 43 | 223231_at | CDA11 | CDA11 protein | 8q24.13 | Exemplarsequence | Hs.11810.0 | AF212250.1 | g13182774 | GenBank |
| 44 | 204278_s_at | EBAG9 | estrogen receptor binding site | 8q23 | Exemplarsequence | Hs.9222.0 | NM_004215.1 | g4758229 | RefSeq |
| | | | associated, antigen, 9 | | | | | | |
| 45 | 218059_at | LOC51123 | HSPCO38 protein | 8q22.3 | Exemplarsequence | Hs.23528.0 | NM_016096.1 | g7705816 | RefSeq |
| 46 | 201433_s_at | PTDSS1 | phosphatidylserine synthase 1 | 8q22 | Exemplarsequence | Hs.77329.0 | NM_014754.1 | g7662646 | RefSeq |
| 47 | 224743_at | | Homo sapiens, clone | | Consensussequence | Hs.13328.0 | BF965065 | Hs.13328.0_RC | GenBank |
| | | | IMAGE: 3897094, mRNA | | | | | | |
| 48 | 210950_s_at | FDFT1 | farnesyl-diphosphate | 8p23.1-p22 | Exemplarsequence | Hs.48876.0 | BC003573.1 | g13097746 | GenBank |
| | | | farnesyltransferase 1 | | | | | | |
| 49 | 209471_s_at | FNTA | farnesyltransferase, CAAX box, | 8p22-q11 | Exemplarsequence | Hs.138381.0 | L00634.1 | g292030 | GenBank |
| | | | alpha | | | | | | |
| 50 | 225378_at | FLJ32642 | hypothetical protein FLJ32642 | 8p22 | Consensussequence | Hs.101617.0 | AI866426 | Hs.101617.0_RC | GenBank |
| 51 | 228024_at | FLJ32642 | hypothetical protein FLJ32642 | 8p22 | Consensussequence | Hs.290855.0 | AW028100 | Hs.290855.0 | GenBank |
| 52 | 201985_at | KIAA0196 | KIAA0196 gene product | 8p22 | Exemplarsequence | Hs.8294.0 | NM_014846.1 | g7661987 | RefSeq |
| 53 | 207000_s_at | PPP3CC | protein phosphatase 3 (formerly | 8p21.2 | Exemplarsequence | Hs.75206.0 | NM_005605.1 | g5031988 | RefSeq |
| | | | 2B), catalytic subunit, gamma | | | | | | |
| | | | isoform (calcineurin A gamma) | | | | | | |
| 54 | 227075_at | ELP3 | likely ortholog of mouse | 8q21.2 | Consensussequence | Hs.267905.2 | AI949204 | Hs.267905.2_RC | GenBank |
| | | | elongation protein 3 homolog (S. cerevisiae) | | | | | | |
| 55 | 209384_at | PROSC | proline synthetase co- | 8p11.2 | Consensussequence | Hs.301959.0 | AA176833 | Hs.301959.0.A2 | GenBank |
| | | | transcribed homolog (bacterial) | | | | | | |
| 56 | 218017_s_at | FLJ22242 | hypothetical protein FLJ22242 | 8p11.1 | Exemplarsequence | Hs.288057.0 | NM_025070.1 | g13376612 | RefSeq |
| 57 | 202872_at | ATP6V1C1 | ATPase, H+ transporting, | 8q22.3 | Consensussequence | Hs.86905.0 | NM_001695.1 | Hs.86905.0 | GenBank |
| | | | lysosomal 42 kDa, V1 subunit C, | | | | | | |
| | | | isoform 1 | | | | | | |
| 58 | 209066_x_at | UQCRB | ubiquinol-cytochrome c | 8q22 | Exemplarsequence | Hs.131255.1 | M26700.1 | g190803 | GenBank |
| | | | reductase binding protein | | | | | | |
| 59 | 218549_s_at | CGI-90 | CGI-90 protein | 8q21.13 | Exemplarsequence | Hs.44222.0 | NM_016033.1 | g7705802 | RefSeq |
| 60 | 201652_at | COPS5 | COP9 constitutive | 8q12.3 | Exemplarsequence | Hs.198767.0 | NM_006837.1 | g5803045 | RefSeq |
| | | | photomorphogenic homolog | | | | | | |
| | | | subunit 5 (Arabidopsis) | | | | | | |
| 61 | 239877_at | MTMR9 | myotubularin related protein 9 | 8p23-p22 | Consensussequence | Hs.128277.0 | AI499833 | Hs.128277.0_RC | GenBank |
| 62 | 203941_at | FLJ10871 | hypothetical protein FLJ10871 | 8p12 | Exemplarsequence | Hs.15562.0 | NM_018250.1 | g8922725 | RefSeq |
| 63 | 215983_s_at | D8S2298E | reproduction 8 | 8p12-p11.2 | Consensussequence | Hs.153678.1 | D83768.1 | Hs.153678.1.S1 | GenBank |
| 64 | 208846_s_at | VDAC3 | voltage-dependent anion | 8p11.2 | Exemplarsequence | Hs.7381.0 | U90943.1 | g2735306 | GenBank |
| | | | channel 3 | | | | | | |
| 65 | 214394_x_at | EEF1D | eukaryotic translation elongation | 8q24.3 | Consensussequence | Hs.223241.1 | AI613383 | Hs.223241.1.A1 | GenBank |
| | | | factor 1 delta (guanine | | | | | | |
| | | | nucleotide exchange protein) | | | | | | |
| 66 | 219189_at | FBXL6 | F-box and leucine-rich repeat | 8q24.3 | Exemplarsequence | Hs.12271.0 | NM_024555.1 | g13435140 | RefSeq |
| | | | protein 6 | | | | | | |
| 67 | 211060_x_at | GPAA1 | GPAA1P anchor attachment | 8q24.3 | Exemplarsequence | g13623546 | BC006383.1 | g13623546 | GenBank |
| | | | protein 1 homolog (yeast) | | | | | | |
| 68 | 212090_at | VPS28 | vacuolar protein sorting 28 | 8q24.3 | Consensussequence | Hs.101067.2 | AL571424 | Hs.101067.2 | GenBank |
| | | | (yeast) | | | | | | |
| 69 | 227778_at | | Homo sapiens cDNA FLJ35542 | | Consensussequence | Hs.26563.0 | H11075 | Hs.26563.0_RC | GenBank |
| | | | fis, clone SPLEN2002917. | | | | | | |
| 70 | 212556_at | SCRIB | scribble | 8q24.3 | Consensussequence | Hs.239784.0 | AI469403 | Hs.239784.0.S1 | GenBank |
| 71 | 36936_at | TSTA3 | tissue specific transplantation | 8q24.3 | Consensussequence | 4900667_rc | U58766 | 4900667_rc | GenBank |
| | | | antigen P35B | | | | | | |
| 72 | 222133_s_at | CGI-72 | CGI-72 protein | 8q24.3 | Consensussequence | Hs.288435.0 | AK022280.1 | Hs.288435.0 | GenBank |
| 73 | 226942_at | FLJ21615 | hypothetical protein FLJ21615 | 8q24.22 | Consensussequence | Hs.44159.0 | AI742668 | Hs.44159.0.S1 | GenBank |
| 74 | 227523_s_at | CGI-72 | CGI-72 protein | 8q24.3 | Consensussequence | Hs.318725.1 | AA192936 | Hs.318725.1.SI | GenBank |
| 75 | 224790_at | DDEF1 | development and differentiation | 8q24.1-q24.2 | Consensussequence | Hs.10669.1 | W03103 | Hs.10669.1.S2 | GenBank |
| | | | enhancing factor 1 | | | | | | |
| 76 | 224796_at | DDEF1 | development and differentiation | 8q24.1-q24.2 | Consensussequence | Hs.10669.1 | W03103 | Hs.10669.1.S2 | GenBank |
| | | | enhancing factor 1 | | | | | | |
| 77 | 226536_at | FLJ32440 | hypothetical protein FLJ32440 | 8q24.13 | Consensussequence | Hs.58609.0 | AL562908 | Hs.58609.0.S1 | GenBank |
| 78 | 208608_s_at | SNTB1 | syntrophin, beta 1 (dystrophin- | 8q23-q24 | Exemplarsequence | Hs.95011.0 | NM_021021.1 | g11321639 | RefSeq |
| | | | associated protein A1, 59 kDa, | | | | | | |
| | | | basic component 1) | | | | | | |
| 79 | 226438_at | | Homo sapiens cDNA: FLJ21447 | | Consensussequence | Hs.44241.0 | AK025100.1 | Hs.44241.0 | GenBank |
| | | | fis, clone COL04468. | | | | | | |
| 80 | 218482_at | DC6 | DC6 protein | 8q23.2 | Exemplarsequence | Hs.283740.0 | NM_020189.1 | g9910185 | RefSeq |
| 81 | 204274_at | EBAG9 | estrogen receptor binding site | 8q23 | Consensussequence | Hs.9222.0 | AA812215 | Hs.9222.0 | GenBank |
| | | | associated, antigen, 9 | | | | | | |
| 82 | 218197_s_at | OXR1 | oxidation resistance 1 | 8q23 | Exemplarsequence | Hs.169111.0 | NM_018002.1 | g8922240 | RefSeq |
| 83 | 226463_at | | Homo sapiens cDNA FLJ33383 | | Consensussequence | Hs.290880.0 | AW241758 | Hs.290880.0.A1 | GenBank |
| | | | fis, clone BRACE2006514. | | | | | | |
| 84 | 202635_s_at | POLR2K | polymerase (RNA) II (DNA | 8q22.2 | Exemplarsequence | Hs.150675.0 | NM_005034.1 | g4826923 | RefSeq |
| | | | directed) polypeptide K, 7.0 kDa | | | | | | |
| 85 | 208454_s_at | PGCP | plasma glutamate | 8q22.2 | Exemplarsequence | Hs.278993.0 | NM_016134.1 | g7706386 | RefSeq |
| | | | carboxypeptidase | | | | | | |
| 86 | 223110_at | DKFZP434I116 | DKFZP4341116 protein | 8q22.1 | Exemplarsequence | Hs.16621.0 | BC003701.1 | g13277583 | GenBank |
| 87 | 218905_at | FLJ20530 | hypothetical protein FLJ20530 | 8q22.1 | Exemplarsequence | Hs.279521.0 | NM_017864.1 | g8923495 | RefSeq |
| 88 | 225600_at | | Homo sapiens cDNA FLJ40637 | | Consensussequence | Hs.6390.1 | AW303300 | Hs.6390.1.A1 | GenBank |
| | | | fis, clone THYMU2015984. | | | | | | |
| 89 | 225603_s_at | | Homo sapiens cDNA FLJ40637 | | Consensussequence | Hs.6390.1 | AW303300 | Hs.6390.1.A1 | GenBank |
| | | | fis, clone THYMU2015984. | | | | | | |
| 90 | 212251_at | | Homo sapiens, Similar to LYRIC, | | Consensussequence | Hs.243901.0 | AI972475 | Hs.243901.0.S1 | GenBank |
| | | | clone MGC: 41931 | | | | | | |
| | | | IMAGE: 5298467, mRNA, | | | | | | |
| | | | complete cds | | | | | | |
| 91 | 222699_s_at | FLJ13187 | phafin 2 | 8q22.1 | Consensussequence | Hs.29724.0 | BF439250 | Hs.29724.0.S1 | GenBank |
| 92 | 203790_s_at | UK114 | translational inhibitor protein | 8q22 | Consensussequence | Hs.18426.0 | N54448 | Hs.18426.0.S1 | GenBank |
| | | | p14.5 | | | | | | |
| 93 | 209065_at | UQCRB | ubiquinol-cytochrome c | 8q22 | Exemplarsequence | Hs.131255.1 | BC005230.1 | g13528857 | GenBank |
| | | | reductase binding protein | | | | | | |
| 94 | 202118_s_at | CPNE3 | copine III | 8q21.13 | Consensussequence | Hs.14158.0 | AA541758 | Hs.14158.0 | GenBank |
| 95 | 210296_s_at | PXMP3 | peroxisomal membrane protein | 8q21.1 | Exemplarsequence | Hs.180612.1 | BC005375.1 | g13529226 | GenBank |
| | | | 3, 35 kDa (Zellweger syndrome) | | | | | | |
| 96 | 202905_x_at | NBS1 | Nijmegen breakage syndrome 1 | 8q21-q24 | Consensussequence | Hs.25812.0 | AI796269 | Hs.25812.0.S1 | GenBank |
| | | | (nibrin) | | | | | | |
| 97 | 202907_s_at | NBS1 | Nijmegen breakage syndrome 1 | 8q21-q24 | Exemplarsequence | Hs.25812.0 | NM_002485.2 | g6996019 | RefSeq |
| | | | (nibrin) | | | | | | |
| 98 | 204226_at | STAU2 | staufen, RNA binding protein, | 8q13-q21.1 | Exemplarsequence | Hs.96870.0 | NM_014393.1 | g7657624 | RefSeq |
| | | | homolog 2 (Drosophila) | | | | | | |
| 99 | 219449_s_at | FLJ20533 | hypothetical protein FLJ20533 | 8q13.3 | Exemplarsequence | Hs.106650.0 | NM_017866.1 | g8923499 | RefSeq |
| 100 | 201399_s_at | TRAM | translocating chain-associating | 8q13.1 | Exemplarsequence | Hs.4147.0 | NM_014294.1 | g7657654 | RefSeq |
| | | | membrane protein | | | | | | |
| 101 | 202956_at | BIG1 | brefeldin A-inhibited guanine | 8q13 | Exemplarsequence | Hs.94631.0 | NM_006421.2 | g6715588 | RefSeq |
| | | | nucleotide-exchange protein 1 | | | | | | |
| 102 | 216266_s_at | BIG1 | brefeldin A-inhibited guanine | 8q13 | Consensussequence | Hs.94631.1 | AK025637.1 | Hs.94631.1.S1 | GenBank |
| | | | nucleotide-exchange protein 1 | | | | | | |
| 103 | 220038_at | SGKL | serum/glucocorticoid regulated | 8q12.3-8q13.1 | Exemplarsequence | Hs.279696.0 | NM_013257.1 | g7019526 | RefSeq |
| | | | kinase-like | | | | | | |
| 104 | 227627_at | SGKL | serum/glucocorticoid regulated | 8q12.3-8q13.1 | Consensussequence | Hs.24131.0 | AV690866 | Hs.24131.0.S1 | GenBank |
| | | | kinase-like | | | | | | |
| 105 | 218185_s_at | FLJ10511 | hypothetical protein FLJ10511 | 8q12.2 | Exemplarsequence | Hs.106768.0 | NM_018120.1 | g8922478 | RefSeq |
| 106 | 210896_s_at | ASPH | aspartate beta-hydroxytase | 8q12.1 | Exemplarsequence | Hs.283664.4 | AF306765.1 | g11991236 | GenBank |
| 107 | 208731_at | RAB2 | RAB2, member RAS oncogene | 8q12.1 | Consensussequence | Hs.78305.0 | NM_002865.1 | Hs.78305.0_RC | GenBank |
| | | | family | | | | | | |
| 108 | 208734_x_at | RAB2 | RAB2, member RAS oncogene | 8q12.1 | Exemplarsequence | Hs.78305.0 | M28213.1 | g550061 | GenBank |
| | | | family | | | | | | |
| 109 | 222701_s_at | MGC2217 | hypothetical protein MGC2217 | 8q11.23 | Consensussequence | Hs.323164.0 | AA570393 | Hs.323164.0 | GenBank |
| 110 | 209096_at | UBE2V2 | ubiquitin-conjugating enzyme E2 | 8q11.1 | Exemplarsequence | Hs.79300.0 | U62136.2 | g4775663 | GenBank |
| | | | variant 2 | | | | | | |
| 111 | 219281_at | MSRA | methionine sulfoxide reductase A | 8p23.1 | Exemplarsequence | Hs.26458.0 | NM_012331.2 | g13259538 | RefSeq |
| 112 | 221504_s_at | ATP6V1H | ATPase, H+ transporting, | 8p22-q22.3 | Exemplarsequence | Hs.19576.0 | AF112204.1 | g6563195 | GenBank |
| | | | lysosomal 50/57 kDa, V1 subunit H | | | | | | |
| 113 | 200762_at | DPYSL2 | dihydropyrimidinase-like 2 | 8p22-p21 | Exemplarsequence | Hs.173381.0 | NM_001386.1 | g4503376 | RefSeq |
| 114 | 212866_at | | Homo sapiens, simliar to | | Consensussequence | Hs.300861.0 | AI081543 | Hs.300861.0 | GenBank |
| | | | hypothetical protein | | | | | | |
| | | | DKFZp564N123.1 - human | | | | | | |
| | | | (fragment), clone | | | | | | |
| | | | IMAGE: 5220614, mRNA | | | | | | |
| 115 | 221094_s_at | ELP3 | likely ortholog of mouse | 8p21.1 | Exemplarsequence | Hs.267905.0 | NM_018091.1 | g8922417 | RefSeq |
| | | | elongation protein 3 homolog (S. cerevisiae) | | | | | | |
| 116 | 225609_at | GSR | glutathione reductase | 8p21.1 | Consensussequence | Hs.193974.0 | AI888037 | Hs.193974.0.S1 | GenBank |
| 117 | 227102_at | TRIM35 | tripartite motif-containing 35 | 8p21.1 | Consensussequence | Hs.137732.0 | AA115933 | Hs.137732.0 | GenBank |
| 118 | 218955_at | BRF2 | BRF2, subunit of RNA | 8p11.1 | Exemplarsequence | Hs.274136.0 | NM_018310.1 | g8922843 | RefSeq |
| | | | polymerase III transcription | | | | | | |
| | | | initiation factor, BRF1-like | | | | | | |
| 119 | 220985_s_at | DKFZP564A022 | hypothetical protein | 8p11.1 | Exemplarsequence | g13569925 | NM_030954.1 | g13569925 | RefSeq |
| | | | DKFZp564A022 | | | | | | |
| 120 | 224776_at | DKFZp586M1819 | hypothetical protein | 8p11.1 | Consensussequence | Hs.25664.1 | BF513102 | Hs.25664.1.A1 | GenBank |
| | | | DKFZp586M1819 | | | | | | |
| 121 | 221542_s_at | C8orf2 | chromosome 8 open reading | 8p11.2 | Consensussequence | Hs.125849.0 | T90773 | Hs.125849.0 | GenBank |
| | | | frame 2 | | | | | | |
| 122 | 221543_s_at | C8orf2 | chromosome 8 open reading | 8p11.2 | Exemplarsequence | Hs.125849.0 | AL442077.1 | g10241715 | GenBank |
| | | | frame 2 | | | | | | |
| 123 | 209341_s_at | IKBKB | inhibitor of kappa light | 8p11.2 | Consensussequence | Hs.226573.0 | AU153366 | Hs.226573.0 | GenBank |
| | | | polypeptide gene enhancer in B- | | | | | | |
| | | | cells, kinase beta | | | | | | |
| 124 | 209385_s_at | PROSC | proline synthetase co- | 8p11.2 | Exemplarsequence | Hs.301959.0 | AL136616.1 | g12052757 | GenBank |
| | | | transcribed homolog (bacterial) | | | | | | |
| 125 | 219416_at | SCARA3 | scavenger receptor class A, | 8p21 | Exemplarsequence | Hs.128856.0 | NM_016240.1 | g7705335 | RefSeq |
| | | | member 3 | | | | | | |
| 126 | 228013_at | | Homo sapiens mRNA; cDNA | | Consensussequence | Hs.61696.0 | AV702575 | Hs.61696.0_RC | GenBank |
| | | | DKFZp586F1523 (from clone | | | | | | |
| | | | DKFZp586F1523) | | | | | | |
| 127 | 230361_at | | ESTs, Weakly similar to inner | | Consensussequence | Hs.146109.0 | AW664013 | Hs.146109.0_RC | GenBank |
| | | | centromere protein [Mus | | | | | | |
| | | | musculus] [M. musculus] | | | | | | |
| 128 | 219060_at | FLJ10204 | hypothetical protein FLJ10204 | 8q24.13 | Exemplarsequence | Hs.18029.0 | NM_018024.1 | g8922280 | RefSeq |
| 129 | 219231_at | NCOA6IP | nuclear receptor coactivator 6 | 8q11 | Exemplarsequance | Hs.179909.0 | NM_024831.1 | g13376235 | RefSeq |
| | | | interacting protein | | | | | | |
| 130 | 227017_at | LOC157697 | hypothetical protein LOC157697 | 8p23.3 | Consensussequence | Hs.193090.0 | AA767385 | Hs.193090.0.A1 | GenBank |
| 131 | 218337_at | RAI16 | retinoic acid induced 16 | 8p21.2 | Exemplarsequence | Hs.299148.0 | NM_022749.1 | g12232410 | RefSeq |
| 132 | 209253_at | SCAM-1 | vinexin beta (SH3-containing | 8p21.2 | Exemplarsequence | Hs.33787.1 | AF037261.1 | g3004947 | GenBank |
| | | | adaptor molecule-1) | | | | | | |
| 133 | 227836_at | | | | Consensussequence | Hs.86970.0 | AI859767 | Hs.86970.0.A1 | GenBank |
| 134 | 221020_s_at | MFTC | mitochondrial folate | 8q22.3 | Exemplarsequence | g13540550 | NM_030780.1 | g13540550 | RefSeq |
| | | | transporter/carrier | | | | | | |
| 135 | 228225_at | PXMP3 | peroxisomal membrane protein | 8q21.1 | Consensussequence | Hs.180612.2 | AW512586 | Hs.180612.2.A1 | GenBank |
| | | | 3, 35 kDa (Zellweger syndrome) | | | | | | |
| 136 | 213139_at | SNAI2 | snail homolog 2 (Drosophila) | 8q11 | Consensussequence | Hs.93005.0 | AI572079 | Hs.93005.0 | GenBank |
| 137 | 220843_s_at | DKFZP564O0463 | DKFZP564O0463 protein | 8q22.3 | Exemplarsequence | Hs.273344.0 | NM_014156.1 | g7661767 | RefSeq |
| 138 | 203534_at | LSM1 | LSM1 homolog, U6 small | 8p11.2 | Exemplarsequence | Hs.111783.0 | NM_014462.1 | g7657312 | RefSeq |
| | | | nuclear RNA associated (S. cerevisiae) | | | | | | |
| 139 | 209510_at | TRC8 | patched related protein | 8q24 | Exemplarsequence | Hs.28285.0 | AF064801.1 | g3395786 | GenBank |
| | | | translocated in renal cancer | | | | | | |
| 140 | 209295_at | TNFRSF10B | tumor necrosis factor receptor | 8p22-p21 | Exemplarsequence | Hs.51233.0 | AF016266.1 | g2529562 | GenBank |
| | | | superfamily, member 10b | | | | | | |
| 141 | 224218_s_at | TRPS1 | trichorhinophalangeal syndrome I | 8q24.12 | Exemplarsequence | Hs.26102.1 | AF264784.1 | g10644121 | GenBank |
| 142 | 231764_at | CHRAC1 | chromatin accessibility complex 1 | 8q24.3 | Consensussequence | Hs.279704.0 | AK023537.1 | Hs.279704.0 | GenBank |
| 143 | 219071_x_at | LOC51236 | hypothetical protein LOC51236 | 8q24.3 | Exemplarsequence | Hs.300224.0 | NM_016458.2 | g13124772 | RefSeq |
| 144 | 226707_at | PP3856 | similar to CG3714 gene Product | 8q24.3 | Consensussequence | Hs.9614.3 | BE870868 | Hs.9614.3_RC | GenBank |
| 145 | 212149_at | KIAA0143 | KIAA0143 protein | 8q24.22 | Consensussequence | Hs.84087.0 | AA805651 | Hs.84087.0.S1 | GenBank |
| 146 | 212150_at | KIAA0143 | KIAA0143 protein | 8q24.22 | Consensussequence | Hs.84087.0 | AA805651 | Hs.84087.0.S1 | GenBank |
| 147 | 234351_x_at | TRPS1 | trichorhinophalangeal syndrome I | 8q24.12 | Consensussequence | Hs.26102.2 | AK000948.1 | Hs.26102.2 | GenBank |
| 148 | 219793_at | SNX16 | sorting nexin 16 | 8q21.12 | Exemplarsequence | Hs.128645.0 | NM_022133.1 | g11545864 | RefSeq |
| 149 | 225622_at | PAG | Phosphoprotein associated with | 8q21.11 | Consensussequence | Hs.266175.0 | NM_018440.1 | Hs.266175.0 | GenBank |
| | | | glycosphingolipid-enriched | | | | | | |
| | | | microdomains | | | | | | |
| 150 | 202824_s_at | TCEB1 | transcription elongation factor B | 8q13.3 | Exemplarsequence | Hs.184693.O | NM_005648.1 | g5032160 | RefSeq |
| | | | (SIII), polypeptide 1 (15 kDa, | | | | | | |
| | | | elongin C) | | | | | | |
| 151 | 202313_at | PPP2R2A | protein phosphataes 2 (formerly | 8p21.1 | Exemplarsequence | Hs.179574.0 | NM_002717.1 | g4506018 | RefSeq |
| | | | 2A), regulatory subunit B (PR | | | | | | |
| | | | 52), alpha isoform | | | | | | |
| 152 | 236192_at | | ESTs | | Consensussequence | Hs.124961.0 | BF447112 | Hs.124961.0_RC | GenBank |
| 153 | 217437_s_at | TACC1 | transforming, acidic coiled-coil | 8p11 | Consensussequence | Hs.173159.1 | AB029026.1 | Hs.173159.1.S1 | GenBank |
| | | | containing protein 1 | | | | | | |
| 154 | 202344_at | HSF1 | heat shock transcription factor 1 | 8q24.3 | Exemplarsequence | Hs.1499.0 | NM_005526.1 | g5031766 | RefSeq |
| 155 | 209899_s_at | SIAHBP1 | fuse-binding protein-interacting | 8q24.2-qtel | Exemplarsequence | Hs.74562.0 | AF217197.1 | g6740005 | GenBank |
| | | | repressor | | | | | | |
| 156 | 33132_at | CPSF1 | cleavage and polyadenylation | 8q24.23 | Consensussequence | 8 | U37012 | 4923232 | GenBank |
| | | | specific factor 1, 160 kDa | | | | | | |
| 157 | 218096_at | FLJ11210 | hypothetical protein FLJ11210 | 8p23.1 | Exemplarsequence | Hs.27842.0 | NM_018361.1 | g8922941 | RefSeq |
| 158 | 214054_at | DOK2 | docking protein 2, 56 kDa | 8p21.2 | Consensussequence | Hs.71215.0 | AI828929 | Hs.71215.0 | GenBank |
| 159 | 207287_at | FLJ14107 | hypotheticai protein FLJ14107 | 8p21.2 | Exemplarsequence | Hs.287624.0 | NM_025026.1 | g13376547 | RefSeq |
| 160 | 218151_x_at | FLJ11856 | putative G-protein coupled | 8q24.3 | Exemplarsequence | Hs.6459.0 | NM_024531.1 | g13375681 | RefSeq |
| | | | receptor GPCR41 | | | | | | |
| 161 | 219402_s_at | MGC3067 | hypothetical protein MGC3067 | 8q24.13 | Exemplarsequence | Hs.323114.0 | NM_024295.1 | g13236515 | RefSeq |
| 162 | 201592_at | EIF3S3 | eukaryotic translation initiation | 8q23.3 | Exemplarsequence | Hs.58189.O | NM_003756.1 | g4503514 | RefSeq |
| | | | factor 3, subunit 3 gamma. | | | | | | |
| | | | 40 kDa | | | | | | |
| 163 | 226776_at | DC6 | DC6 protein | 8q23.2 | Consensussequence | Hs.44243.0 | BF215862 | Hs.44243.0_RC | GenBank |
| 164 | 208697_s_at | EIF3S6 | eukaryotic translation initiation | 8q22-q23 | Exemplarsequence | Hs.106673.0 | BC000734.1 | g12653884 | GenBank |
| | | | factor 3, subunit 6 48 kDa | | | | | | |
| 165 | 203501_at | PGCP | plasma glutamate | 8q22.2 | Exemplarsequence | Hs.197335.0 | NM_006102.1 | g5174626 | RefSeq |
| | | | carboxypeptidase | | | | | | |
| 166 | 203011_at | IMPA1 | inositol(myo)-1 (or 4)- | 8q21.13-q21.3 | Exemplarsequence | Hs.171776.0 | NM_005536.2 | g8393607 | RefSeq |
| | | | monophosphatase 1 | | | | | | |
| 167 | 219810_at | VCIP135 | valosin-containing protein | 8q13 | Exemplarsequence | Hs.287727.0 | NM_025054.1 | g13376584 | RefSeq |
| | | | (p97)/p47 complex-interacting | | | | | | |
| | | | protein p135 | | | | | | |
| 168 | 221749_at | FLJ31657 | hypothetical protein FLJ31657 | 8q12.1 | Consensussequence | Hs.5518.0 | AU157915 | Hs.5518.0.S1 | GenBank |
| 169 | 212449_s_at | LYPLA1 | lysophosphoilpase 1 | 8q11.23 | Consensussequence | Hs.12540.2 | BG288007 | Hs.12540.2_RC | GenBank |
| 170 | 219340_s_at | CLN8 | ceroid-lipofuscinosis, neuronal 8 | 8p23 | Exemplarsequence | Hs.127675.0 | AF123759.1 | g6467264 | GenBank |
| | | | (epilepsy, progressive with | | | | | | |
| | | | mental retardation) | | | | | | |
| 171 | 213702_x_at | ASAH1 | N-acylsphingosine | 8p22-p21.3 | Consensussequence | Hs.75811.3 | AI934569 | Hs.75811.3.S1 | GenBank |
| | | | amidohydrolase (acid | | | | | | |
| | | | ceramidase) 1 | | | | | | |
| 172 | 211686_s_at | LOC84549 | RNA binding protein | 8p11.23 | Exemplarsequence | g13625185 | AF251062.1 | g13625185 | GenBank |
| 173 | 200847_s_at | MGC8721 | hypothetical protein MGC8721 | 8p12 | Exemplarsequence | Hs.279921.0 | NM_016127.1 | g7706384 | RefSeq |
| 174 | 219897_at | FLJ12526 | hypothetical protein FLJ12526 | 8p11.23 | Exemplarsequence | Hs.151237.0 | NM_024787.1 | g13376151 | RefSeq |
| 175 | 219624_at | BAG4 | BCL2-associated athanogene 4 | 8p11.21 | Exemplarsequence | Hs.194726.0 | NM_004874.1 | g6631074 | RefSeq |
| 176 | 219292_at | FLJ10477 | hypothetical protein FLJ10477 | 8p11.1 | Exemplarsequence | Hs.7432.0 | NM_018105.1 | g8922445 | RefSeq |
| 177 | 205089_at | ZNF7 | zinc finger protein 7 (KOX 4, | 8q24 | Exemplarsequence | Hs.2076.0 | NM_003416.1 | g4508034 | RefSeq |
| | | | clone HF.16) | | | | | | |
| 178 | 201066_at | CYC1 | cytochrome c-1 | 8q24.3 | Exemplarsequence | Hs.289271.0 | NM_001916.1 | g4503184 | RefSeq |
| 179 | 209523_at | TAF2 | TAF2 RNA polymerase II, TATA | 8q24.12 | Consensussequence | Hs.122752.0 | AK001618.1 | Hs.122752.0 | GenBank |
| | | | box binding protein (TBP)- | | | | | | |
| | | | associated factor, 150 kDa | | | | | | |
| 180 | 200608_s_at | RAD21 | RAD21 homolog (S. pombe) | 8q24 | Exemplarsequence | Hs.81848.0 | NM_006265.1 | g5453993 | RefSeq |
| 181 | 204068_at | STK3 | serine/threonine kinase 3 | 8q22.1 | Exemplarsequence | Hs.166684.0 | NM_006281.1 | g5454093 | RefSeq |
| | | | (STE20 homolog, yeast) | | | | | | |
| 182 | 208882_s_at | DD5 | progestin induced protein | 8q22 | Consensussequence | Hs.278428.0 | U69567 | Hs.278428.0.A2 | GenBank |
| 183 | 212637_s_at | WWP1 | WW domain-containing protein 1 | 8q21 | Consensussequence | Hs.324275.0 | BF131791 | Hs.324275.0.S1 | GenBank |
| 184 | 218027_at | MRPLI5 | mitochondrial ribosomal protein | 8q11.2-q13 | Exemplarsequence | Hs.18349.0 | NM_014175.1 | g7661805 | RefSeq |
| | | | L15 | | | | | | |
| 185 | 200090_at- | FNTA | farnesyltransferase, CAAX box, | 8p22-q11 | Consensussequence | Hs.138381.1 | BG168896 | Hs.138381.1.A1 | GenBank |
| | HG-U133B | | alpha | | | | | | |
| 186 | 201089_at | ATP6V1B2 | ATPase, H+ transporting, | 8p22-p21 | Exemplarsequence | Hs.1697.0 | NM_001693.1 | g4502310 | RefSeq |
| | | | lysosomal 56/58 kDa, V1 subunit | | | | | | |
| | | | B, isoform 2 | | | | | | |
| 187 | 32541_at | PPP3CC | protein phosphatase 3 (formerly | 8p21.2 | Consensussequence | 6 | S46622 | 4922761 | GenBank |
| | | | 2B), catalytic subunit, gamma | | | | | | |
| | | | isoform (calcineurin A gamma) | | | | | | |
| 188 | 202962_at | KIF13B | kinesin family member 13B | 8p12 | Exemplarsequence | Hs.15711.0 | NM_015254.1 | g13194196 | RefSeq |
| 189 | 201375_s_at | PPP2CB | protein phosphatase 2 (formerly | 8p12-p11.2 | Exemplarsequence | Hs.80350.0 | NM_004156.1 | g4758951 | RefSeq |
| | | | 2A), catalytic subunit, beta | | | | | | |
| | | | isoform | | | | | | |
| 190 | 221539_at | EIF4EBP1 | eukaryotic translation initiation | 8p12 | Exemplarsequence | Hs.71819.0 | AB044548.1 | g11527778 | GenBank |
| | | | factor 4E binding protein 1 | | | | | | |
| 191 | 212690_at | KIAA0725 | KIAA0725 protein | 8p11.21 | Consensussequence | Hs.26450.0 | AB018268.1 | Hs.26450.0_RC | GenBank |
| 192 | 225534_at | LOC114926 | hypothetical protein BC013035 | 8p11.1 | Consensussequence | Hs.10018.1 | AV711345 | Hs.10018.1.A1 | GenBank |
| 193 | 203616_at | POLB | polymerase (DNA directed), beta | 8p11.2 | Exemplarsequence | Hs.180107.0 | NM_002690.1 | g4505930 | RefSeq |
| 194 | 224076_s_at | WHSC1L1 | Wolf-Hirschhom syndrome | 8p11.2 | Exemplarsequence | Hs.27721.1 | AF255649.1 | g12005822 | GenBank |
| | | | candidate 1-like 1 | | | | | | |
| 195 | 200632_s_at | NDRG1 | N-myc downstream regulated | 8q24 | Exemplarsequence | Hs.75789.0 | NM_006096.1 | g5174656 | RefSeq |
| | | | gene 1 | | | | | | |
| 196 | 201754_at | COX6C | cytochrome c oxidase subunit | 8q22-q23 | Exemplarsequence | Hs.74649.0 | NM_004374.1 | g4758039 | RefSeq |
| | | | VIc | | | | | | |
| 197 | 202634_at | POLR2k | polymerase (RNA) II (DNA | 8q22.2 | Consensussequence | Hs.150675.0 | AL558030 | Hs.150675.0 | GenBank |
| | | | directed) polypeptide K, 7.0 kDa | | | | | | |
| 198 | 202447_at | DECR1 | 2,4-dienoyl CoA reductase 1, | 8q21.3 | Exemplarsequence | Hs.81548.0 | NM_001359.1 | g4503300 | RefSeq |
| | | | mitochondrial | | | | | | |
| 199 | 222036_s_at | MCM4 | MCM4 minichromosome | 8q12-q13 | Consensussequence | Hs.154443.1 | AI859865 | Hs.154443.1.S1 | GenBank |
| | | | maintenance deficient 4 (S. cerevisiae) | | | | | | |
| 200 | 209294_x_at | TNFRSF10B | tumor necrosis factor receptor | 8p22-p21 | Exemplarsequence | Hs.51233.0 | BC001281.1 | g12654874 | GenBank |
| | | | superfamily, member 10b | | | | | | |
| 201 | 209227_at | N33 | Putative prostate cancer tumor | 8p22 | Consensussequence | Hs.71119.0 | AU158251 | Hs.71119.0.A2 | GenBank |
| | | | suppressor | | | | | | |
| 202 | 203669_s_at | DGAT1 | diacylglycerol O-acyltransferase | 8qter | Exemplarsequence | Hs.288627.0 | NM_012079.2 | g7382489 | RefSeq |
| | | | homolog 1 (mouse) | | | | | | |
| 203 | 229350_x_at | FLJ14464 | hypothetical protein FLJ14464 | 8q24.3 | Consensussequence | Hs.135106.0 | AI335251 | Hs.135106.0.A1 | GenBank |
| 204 | 215690_x_at | GPAA1 | GPAA1P anchor attachment | 8q24.3 | Consensussequence | Hs.4742.1 | AL157437.1 | Hs.4742.1 | GenBank |
| | | | protein 1 homolog (yeast) | | | | | | |
| 205 | 212975_at | KIAA0870 | KIAA0870 protein | 8q24.3 | Consensussequence | Hs.18166.0 | AB020677.2 | Hs.18166.0 | GenBank |
| 206 | 219215_s_at | SLC39A4 | solute carrier family 39 (zinc | 8q24.3 | Exemplarsequence | Hs.72289.0 | NM_017767.1 | g8923304 | RefSeq |
| | | | transporter), member 4 | | | | | | |
| 207 | 221836_s_at | MGC4737 | KIAA1882 protein | 8q24.3 | Consensussequence | Hs.157240.1 | AW291218 | Hs.157240.1.S1 | GenBank |
| 208 | 201639_s_at | CPSF1 | cleavage and polyadenylation | 8q24.23 | Exemplarsequence | Hs.83727.0 | NM_013291.1 | g9558724 | RefSeq |
| | | | specific factor 1, 160 kDa | | | | | | |
| 209 | 230098_at | FLJ21615 | hypothetical protein FLJ21615 | 8q24.22 | Consensussequence | Hs.170335.0 | AW612407 | Hs.170335.0_RC | GenBank |
| 210 | 231967_at | CGI-72 | CGI-72 protein | 8q24.3 | Consensussequence | Hs.318725.2 | AI913146 | Hs.318725.2.S1 | GenBank |
| 211 | 208322_s_at | SIAT4A | sialyltransferase 4A (beta- | 8q24.22 | Exemplarsequence | Hs.301698.0 | NM_003033.1 | g4506950 | RefSeq |
| | | | galactoside alpha-2,3- | | | | | | |
| | | | sialyltransferase) | | | | | | |
| 212 | 217916_s_at | BM-009 | hypothetical protein BM-009 | 8q24.21 | Exemplarsequence | Hs.92918.0 | NM_016623.1 | g7705303 | RefSeq |
| 213 | 221039_s_at | DDEF1 | development and differentiation | 8q24.1-q24.2 | Exemplarsequence | Hs.10669.0 | NM_018482.1 | g8923867 | RefSeq |
| | | | enhancing factor 1 | | | | | | |
| 214 | 224791_at | DDEF1 | development and differentiation | 8q24.1-q24.2 | Consensussequence | Hs.10669.1 | W03103 | Hs.10669.1.S2 | GenBank |
| | | | enhancing factor 1 | | | | | | |
| 215 | 234305_s_at | MLZE | melanoma-derived leucine | 8q24.1-q24.2 | Consensussequence | Hs.133244.1 | AJ245876.1 | Hs.133244.1 | GenBank |
| | | | zipper, extra-nuclear factor | | | | | | |
| 216 | 202241_at | C8FW | phosphoprotein regulated by | 8q24.13 | Exemplarsequence | Hs.7837.0 | NM_025195.1 | g13399327 | RefSeq |
| | | | mitogenic pathways | | | | | | |
| 217 | 225702_at | ZHX1 | zinc-fingers and homeoboxes 1 | 8q24.13 | Consensussequence | Hs.12940.1 | AA973041 | Hs.12940.1.A1 | GenBank |
| 218 | 218172_s_at | PRO2577 | hypothetical protein PRO2577 | 8q24.13 | Exemplarsequence | Hs.241576.0 | NM_018630.1 | g8924181 | RefSeq |
| 219 | 218782_s_at | PRO2000 | PRO2000 protein | 8q24.13 | Exemplarsequence | Hs.46677.0 | NM_014109.1 | g7662630 | RefSeq |
| 220 | 223214_s_at | ZHX1 | zinc-fingers and homeoboxes 1 | 8q24.13 | Exemplarsequence | Hs.12940.0 | AF195766.1 | g7012716 | GenBank |
| 221 | 218502_s_at | TRPS1 | trichorhinophalangeal syndrome I | 8q24.12 | Exemplarsequence | Hs.26102.0 | NM_014112.1 | g7657658 | RefSeq |
| 222 | 222651_s_at | TRPS1 | trichorhinophalangeal syndrome I | 8q24.12 | Consensussequence | Hs.26102.0 | BF701166 | Hs.26102.0_RC | GenBank |
| 223 | 227787_s_at | PFDN2 | prefoldin 2 | 1q23.1 | Consensussequence | Hs.298229.2 | AI026938 | Hs.298229.2.A1 | GenBank |
| 224 | 204501_at | NOV | nephroblastoma overexpressed | 8q24.1 | Exemplarsequence | Hs.235935.0 | NM_002514.1 | g4505422 | RefSeq |
| | | | gene | | | | | | |
| 225 | 214321_at | NOV | nephroblastoma overexpressed | 8q24.1 | Consensussequence | Hs.235935.1 | BF440025 | Hs.235935.1.S1 | GenBank |
| | | | gene | | | | | | |
| 226 | 200607_s_at | RAD21 | RAD21 homolog (S. pombe) | 8q24 | Consensussequence | Hs.81848.0 | BG289967 | Hs.81848.0 | GenBank |
| 227 | 226775_at | DC6 | DC6 protein | 8q23.2 | Consensussequence | Hs.44243.0 | BF215862 | Hs.44243.0_RC | GenBank |
| 228 | 223342_at | RRM2B | ribonucleotide reductase M2 B | 8q23.1 | Exemplarsequence | Hs.94262.0 | AB036063.1 | g7229085 | GenBank |
| | | | (TP53 inducible) | | | | | | |
| 229 | 200638_s_at | YWHAZ | tyrosine 3- | 8q23.1 | Exemplarsequence | Hs.75103.0 | BC003623.1 | g13177678 | GenBank |
| | | | monooxygenase/tryptophan 5- | | | | | | |
| | | | monooxygenase activation | | | | | | |
| | | | protein, zeta polypeptide | | | | | | |
| 230 | 200639_s_at | YWHAZ | tyrosine 3- | 8q23.1 | Exemplarsequence | Hs.75103.0 | NM_003406.1 | g4507952 | RefSeq |
| | | | monooxygenase/tryptophan 5- | | | | | | |
| | | | monooxygenase activation | | | | | | |
| | | | protein, zeta polypeptide | | | | | | |
| 231 | 200640_at | YWHAZ | tyrosine 3- | 8q23.1 | Exemplarsequence | Hs.75103.0 | NM_003406.1 | g4507952 | RefSeq |
| | | | monooxygenase/tryptophan 5- | | | | | | |
| | | | monooxygenase activation | | | | | | |
| | | | protein, zeta polypeptide | | | | | | |
| 232 | 200641_s_at | YWHAZ | tyrosine 3- | 8q23.1 | Exemplarsequence | Hs.75103.0 | U28964.1 | g899458 | GenBank |
| | | | monooxygenase/tryptophan 5- | | | | | | |
| | | | monooxygenase activation | | | | | | |
| | | | protein, zeta polypeptide | | | | | | |
| 233 | 236989_at | | ESTs | | Consensussequence | Hs.293171.0 | AW293012 | Hs.293171.0.A1 | GenBank |
| 234 | 203761_at | SLA | Src-like-adaptor | 8q24 | Exemplarsequence | Hs.75367.0 | NM_006748.1 | g5803170 | RefSeq |
| 235 | 211445_x_at | FKSG17 | FKSG17 | 8q22.3 | Exemplarsequence | Hs.307057.0 | AF315951.1 | g12276119 | GenBank |
| 236 | 201772_at | OAZIN | ornithine decarboxylase | 8q22.3 | Exemplarsequence | Hs.223014.0 | NM_015878.1 | g7706219 | RefSeq |
| | | | antizyme inhibitor | | | | | | |
| 237 | 212461_at | OAZIN | ornithine decarboxylase | 8q22.3 | Consensussequence | Hs.278614.1 | BF793951 | Hs.278614.1_RC | GenBank |
| | | | antizyme inhibitor | | | | | | |
| 238 | 210117_at | SPAG1 | sperm associated antigen 1 | 8q22.2 | Exemplarsequence | Hs.153057.0 | AF311312.1 | g10863767 | GenBank |
| 239 | 202393_s_at | TIEG | TGFB inducible early growth | 8q22.2 | Exemplarsequence | Hs.82173.0 | NM_005655.1 | g5032176 | RefSeq |
| | | | response | | | | | | |
| 240 | 218049_s_at | MRPL13 | mitochondrial ribosomal protein | 8q22.1-q22.3 | Exemplarsequence | Hs.43946.0 | NM_014078.1 | g7662495 | RefSeq |
| | | | L13 | | | | | | |
| 241 | 219363_s_at | CGI-12 | CGI-12 protein | 8q22.1 | Exemplarsequence | Hs.46680.0 | NM_015942.1 | g7705587 | RefSeq |
| 242 | 218640_s_at | FLJ13187 | phafin 2 | 8q22.1 | Exemplarsequence | Hs.29724.0 | NM_024613.1 | g13375826 | RefSeq |
| 243 | 208884_s_at | DD5 | progestin induced protein | 8q22 | Exemplarsequence | Hs.278428.0 | AF006010.1 | g4101694 | GenBank |
| 244 | 226338_at | DKFZp762O076 | hypothetical protein | 8q21.3 | Consensussequence | Hs.21621.0 | AA604382 | Hs.21621.0 | GenBank |
| | | | DKFZp762O076 | | | | | | |
| 245 | 225626_at | PAG | phosphoprotein associated with | 8q21.11 | Consensussequence | Hs.266175.0 | NM_018440.1 | Hs.266175.0 | GenBank |
| | | | glycosphingolipid-enriched | | | | | | |
| | | | microdomains | | | | | | |
| 246 | 227354_at | | Homo sapiens cDNA FLJ37858 | | Consensussequence | Hs.13256.0 | BF589359 | Hs.13256.0.A1 | GenBank |
| | | | fis, clone BRSSN2015238. | | | | | | |
| 247 | 209544_at | RIPK2 | receptor-interacting serine- | 8q21 | Exemplarsequence | Hs.103755.0 | AF027706.1 | g3123886 | GenBank |
| | | | threonine kinase 2 | | | | | | |
| 248 | 209545_s_at | RIPK2 | receptor-interacting serine- | 8q21 | Exemplarsequence | Hs.103755.0 | AF064824.1 | g3290171 | GenBank |
| | | | threonine kinase 2 | | | | | | |
| 249 | 212638_s_at | WWP1 | WW domain-containing protein 1 | 8q21 | Consensussequence | Hs.324275.0 | BF131791 | Hs.3242750.S1 | GenBank |
| 250 | 219312_s_at | RiNZF | zinc finger protein RINZF | 8q13-q21.1 | Exemplarsequence | Hs.237146.0 | NM_023929.1 | g12965200 | RefSeq |
| 251 | 218521_s_at | FLJ11011 | hypothetical protein FLJ11011 | 8q13.2 | Exemplarsequence | Hs.21275.0 | NM_018299.1 | g8922821 | RefSeq |
| 252 | 222657_s_at | FLJ11011 | hypothetical protein FLJ11011 | 8q13.2 | Consensussequence | Hs.21275.0 | AK024050.1 | Hs.21275.0.A1 | GenBank |
| 253 | 222992_s_at | NDUPB9 | NADH dehydrogenase | 8q13.3 | Exemplarsequence | Hs.15977.0 | AF261090.1 | g9802311 | GenBank |
| | | | (ubiquinone) 1 beta subcomplex, | | | | | | |
| | | | 9, 22 kDa | | | | | | |
| 254 | 202823_at | TCEB1 | transcription elongation factor B | 8q13.3 | Consensussequence | Hs.184693.0 | N89607 | Hs.184693.0.A2 | GenBank |
| | | | (SIII), polypeptide 1 (15 kDa, | | | | | | |
| | | | elongin C) | | | | | | |
| 255 | 205732_s_at | NCOA2 | nuclear receptor coactivator 2 | 8q13.1 | Exemplarsequence | Hs.29131.0 | NM_006540.1 | g5729857 | RefSeq |
| 256 | 212867_at | NCOA2 | nuclear receptor coactivator 2 | 8q13.1 | Consensussequence | Hs.29131.1 | AI040324 | Hs.29131.1 | GenBank |
| 257 | 220732_at | FLJ12987 | hypothetical protein FLJ12987 | 8q12.3 | Exemplarsequence | Hs.296730.0 | NM_025170.2 | g13489104 | RefSeq |
| 258 | 202625_at | LYN | v-yes-1 Yamaguchi sarcoma | 8q13 | Consensussequence | Hs.80887.0 | AI356412 | Hs.80887.0_RC | GenBank |
| | | | viral related oncogene homolog | | | | | | |
| 259 | 202626_s_at | LYN | v-yes-1 Yamaguchi sarcoma | 8q13 | Exemplarsequence | Hs.80887.0 | NM_002350.1 | g4505054 | RefSeq |
| | | | viral related oncogene homolog | | | | | | |
| 260 | 210754_s_at | LYN | v-yes-1 Yamaguchi sarcoma | 8q13 | Exemplarsequence | Hs.80887.1 | M79321.1 | g187270 | GenBank |
| | | | viral related oncogene homolog | | | | | | |
| 261 | 203449_s_at | TERF1 | telomeric repeat binding factor | 8q13 | Exemplarsequence | Hs.194562.0 | NM_017489.1 | g9257245 | RefSeq |
| | | | (NIMA-interacting) 1 | | | | | | |
| 262 | 203560_at | GGH | gamma-glutamyl hydrolase | 8q12.1 | Exemplarsequence | Hs.78619.0 | NM_003878.1 | g4503986 | RefSeq |
| | | | (conjugase, | | | | | | |
| | | | folylpolygammaglutamyl | | | | | | |
| | | | hydrolase) | | | | | | |
| 263 | 209135_at | ASPH | aspartate beta-hydroxylase | 8q12.1 | Exemplarsequence | Hs.283664.3 | AF289489.1 | g11878115 | GenBank |
| 264 | 218829_s_at | KIAA1416 | KIAA1416 protein | 8q12.1 | Exemplarsequence | Hs.105461.0 | NM_017780.1 | g8923329 | RefSeq |
| 265 | 212934_at | LOC137886 | hypothetical protein LOC137886 | 8q11.23 | Consensussequence | Hs.155572.0 | AI245523 | Hs.155572.0.A1 | GenBank |
| 266 | 208732_at | RAB2 | RAB2, member RAS oncogene | 8q12.1 | Consensussequence | Hs.78305.0 | NM_002865.1 | Hs.78305.0_RC | GenBank |
| | | | family | | | | | | |
| 267 | 208733_at | RAB2 | RAB2, member RAS oncogene | 8q12.1 | Consensussequence | Hs.78305.0 | NM_002865.1 | Hs.78305.0_RC | GenBank |
| | | | family | | | | | | |
| 268 | 221960_s_at | RAB2 | RAB2, member RAS oncogene | 8q12.1 | Consensussequence | Hs.78305.1 | AI189609 | Hs.78305.1.A1 | GenBank |
| | | | family | | | | | | |
| 269 | 205372_at | PLAG1 | pleiomorphic adenoma gene 1 | 8q12 | Exemplarsequence | Hs.14968.0 | NM_002655.1 | g4505854 | RefSeq |
| 270 | 216247_at | RPS20 | ribosomal protein S20 | 8q12 | Consensussequence | Hs.8102.2 | AF113008.1 | Hs.8102.2_RC | GenBank |
| 271 | 200958_s_at | SDCBP | syndecan binding protein | 8q12 | Exemplarsequence | Hs.8180.0 | NM_005625.1 | g5032082 | RefSeq |
| | | | (syntenin) | | | | | | |
| 272 | 222654_at | FLJ20421 | hypothetical protein FLJ20421 | 8q11.23 | Consensussequence | Hs.263727.0 | AW295105 | Hs.263727.0.A1 | GenBank |
| 273 | 203007_s_at | LYPLA1 | lysophospholipase I | 8q11.23 | Exemplarsequence | Hs.12540.0 | AF077198.1 | g4679009 | GenBank |
| 274 | 218642_s_at | MGC2217 | hypothetical protein MGC2217 | 8q11.23 | Exemplarsequence | Hs.323164.0 | NM_024300.1 | g13236525 | RefSeq |
| 275 | 203973_s_at | CEBPD | CCAAT/enhancer binding | 8p11.2-p11.1 | Exemplarsequence | Hs.76722.0 | NM_005195.1 | g4885130 | RefSeq |
| | | | protein (C/EBP), delta | | | | | | |
| 276 | 222037_at | MCM4 | MCM4 minichromosome | 8q12-q13 | Consensussequence | Hs.154443.1 | AI859865 | Hs.154443.1.S1 | GenBenk |
| | | | maintenance deficient 4 (S. cerevisiae) | | | | | | |
| 277 | 208694_at | PRKDC | protein kinase, DNA-activated, | 8q11 | Exemplarsequence | Hs.155637.0 | U47077.5 | g13570016 | GenBank |
| | | | catalytic polypeptide | | | | | | |
| 278 | 210543_s_at | PRKDC | protein kinase, DNA-activated, | 8q11 | Exemplarsequence | Hs.155637.1 | U34994.3 | g13606055 | GenBank |
| | | | catalytic polypeptide | | | | | | |
| 279 | 225591_at | FBXO25 | F-box only protein 25 | 8p23.3 | Consensussequence | Hs.81001.0 | AA749085 | Hs.81001.0.A1 | GenBank |
| 280 | 214440_at | NAT1 | N-acetyltransferase 1 (arylamine | 8p23.1-p21.3 | Consensussequence | Hs.155956.0 | NM_000662.1 | Hs.155956.0.S1 | GenBank |
| | | | N-acetyltransferase) | | | | | | |
| 281 | 226038_at | FLJ23749 | hypothetical protein FLJ23749 | 8p22 | Consensussequence | Hs.180178.0 | BF680438 | Hs.180178.0.S1 | GenBank |
| 282 | 213902_at | ASAH1 | N-acylsphingosine | 8p22-p21.3 | Consensussequence | Hs.75811.2 | AI379338 | Hs.75811.2.S1 | GenBank |
| | | | amidohydrolase (acid | | | | | | |
| | | | ceramidase) 1 | | | | | | |
| 283 | 228905_at | | ESTs | | Consensussequence | Hs.155272.0 | BE672700 | Hs.155272.0_RC | GenBank |
| 284 | 200838_at | CTSB | cathepsin B | 8p22 | Exemplarsequence | Hs.297939.0 | NM_001908.1 | g4503138 | RefSeq |
| 285 | 213275_x_at | CTSB | cathepsin B | 8p22 | Consensussequence | Hs.297939.5 | BE875786 | Hs.297939.5_RC | GenBank |
| 286 | 227961_at | CTSB | cathepsin B | 8p22 | Consensussequence | Hs.297939.2 | AA130998 | Hs.297939.2.A1 | GenBank |
| 287 | 204596_s_at | STC1 | stanniocalcin 1 | 8p21-p11.2 | Exemplarsequence | Hs.25590.0 | U46768.1 | g1199619 | GenBank |
| 288 | 219049_at | ChGn | chondroitin beta1,4 N- | 8p21.3 | Exemplarsequence | Hs.11260.0 | NM_018371.1 | g8922959 | RefSeq |
| | | | acetylgalactosaminyltransferase | | | | | | |
| 289 | 244885_at | | Homo sapiens cDNA FLJ36559 | | Consensussequence | Hs.131056.0 | AI016316 | Hs.131056.0.A1 | GenBank |
| | | | fis, clone TRACH2009291. | | | | | | |
| 290 | 35156_at | | Homo sapiens, similar to | | Consensussequence | 4870467_rc | AL050297 | 4870467_rc | GenBank |
| | | | hypothetical protein | | | | | | |
| | | | DKFZp564N123.1 —human | | | | | | |
| | | | (fragment), clone | | | | | | |
| | | | IMAGE: 5220614, mRNA | | | | | | |
| 291 | 204076_at | LYSAL1 | lysosomal apyrase-like 1 | 8p21.2 | Consensussequence | Hs.201377.0 | AB002390.1 | Hs.201377.0 | GenBank |
| 292 | 219165_at | PDLIM2 | PDZ and LIM domain 2 | 8p21.2 | Exemplarsequence | Hs.19447.0 | NM_021630.1 | g11055999 | RefSeq |
| | | | (mystique) | | | | | | |
| 293 | 218823_s_at | FLJ20038 | hypothetical protein FLJ20038 | 8p21.1 | Exemplarsequence | Hs.72071.0 | NM_017634.1 | g8923043 | RefSeq |
| 294 | 203110_at | PTK2B | PTK2B protein tyrosine kinase 2 | 8p21.1 | Exemplarsequence | Hs.20313.0 | U43522.1 | g1165218 | GenBank |
| | | | beta | | | | | | |
| 295 | 203261_at | DCTN6 | likely ortholog of mouse dynactin 6 | 8p12-p11 | Exemplarsequence | Hs.39913.0 | NM_006571.1 | g5730115 | RefSeq |
| 296 | 65718_at | GPR124 | G protein-coupled receptor 124 | 8p11.1 | Consensussequence | 4885918_rc | AI655903 | 4885918_rc | GenBank |
| 297 | 202381_at | ADAM9 | a disintegrin and | 8p11.21 | Exemplarsequence | Hs.2442.0 | NM_003816.1 | g4501914 | RefSeq |
| | | | metalloproteinase domain 9 | | | | | | |
| | | | (meltrin gamma) | | | | | | |
| 298 | 225136_at | | Homo sapiens cDNA FLJ32643 | | Consensussequence | Hs.18585.0 | BF968578 | Hs.18585.0_RC | GenBank |
| | | | fis, clone SYNOV2001212. | | | | | | |
| 299 | 238615_at | C8orf2 | chromosome 8 open reading | 8p11.2 | Consensussequence | Hs.143848.0 | AI817403 | Hs.143848.0.A1 | GenBank |
| | | | frame 2 | | | | | | |
| 300 | 202423_at | RUNXBP2 | runt-related transcription factor | 8p11 | Exemplarsequence | Hs.82210.0 | NM_006766.1 | g5803097 | RefSeq |
| | | | binding protein 2 | | | | | | |
| 301 | 200911_s_at | TACC1 | transforming, acidic coiled-coil | 8p11 | Exemplarsequence | Hs.173159.0 | NM_006283.1 | g5454099 | RefSeq |
| | | | containing protein 1 | | | | | | |
| 302 | 236034_at | | ESTs | | Consensussequence | Hs.68301.0 | AA083514 | Hs.68301.0.A1 | GenBank |
| 303 | 221586_s_at | E2F5 | E2F transcription factor 5, p130- | 8q21.13 | Exemplarsequence | Hs.2331.0 | U15642.1 | g758415 | GenBank |
| | | | binding | | | | | | |
| 304 | 204597_x_at | STC1 | stanniocalcin 1 | 8p21-p11.2 | Exemplarsequence | Hs.25590.0 | NM_003155.1 | g4507264 | RefSeq |
| 305 | 235209_at | FLJ40021 | hypothetical protein FLJ40021 | 8q13.2 | Consensussequence | Hs.122544.0 | AW662373 | Hs.122544.0_RC | GenBank |
| 306 | 222721_at | HSPC163 | HSPC163 protein | 1q42.12 | Consensussequence | Hs.108854.0 | AK024569.1 | Hs.108854.0.S2 | GenBank |
| 307 | 241355_at | HR | hairless | 8p21.2 | Consensussequence | Hs.165565.0 | BF528433 | Hs.165565.0_RC | GenBank |
| 308 | 202701_at | BMP1 | bone morphogenetic protein 1 | 8p21 | Exemplarsequence | Hs.1274.5 | NM_006129.2 | g5902809 | RefSeq |
| 309 | 228405_at | RHPN1 | rhophilin, Rho GTPase binding | 8q24.3 | Consensussequence | Hs.149152.0 | AI917311 | Hs.149152.0.A1 | GenBank |
| | | | protein 1 | | | | | | |
| 310 | 218954_s_at | BRF2 | BRF2, subunit of RNA | 8p11.1 | Exemplarsequence | Hs.274136.0 | AF298153.1 | g11096174 | GenBank |
| | | | polymerase III transcription | | | | | | |
| | | | initiation factor, BRF1-like | | | | | | |
| 311 | 221814_at | GPR124 | G protein-coupled receptor 124 | 8p11.1 | Consensussequence | Hs.17270.0 | BF511315 | Hs.17270.0 | GenBank |
| 312 | 223614_at | DKFZp761D112 | hypothetical protein | 8q21.2 | Exemplarsequence | Hs.103849.0 | AL136588.1 | g13276678 | GenBank |
| | | | DKFZp761D112 | | | | | | |
| 313 | 243264_s_at | FLJ11267 | hypothetical protein FLJ11267 | 8q12.3 | Consensussequence | Hs.160795.0 | AI634652 | Hs.160795.0_RC | GenBank |
| 314 | 220511_s_at | FLJ21120 | hypothetical protein FLJ21120 | 8p22 | Exemplarsequence | Hs.133546.0 | NM_024767.1 | g13376110 | RefSeq |
| 315 | 206135_at | ST18 | suppression of tumorigenicity 18 | 8q11.22 | Exemplarsequence | Hs.151449.0 | NM_014682.1 | g7662167 | RefSeq |
| | | | (breast carcinoma) (zinc finger | | | | | | |
| | | | protein) | | | | | | |
| 316 | 224021_at | RP1 | retinitis pigmentosa 1 (autosomal | 8q11-q13 | Exemplarsequence | Hs.251687.0 | AF146592.1 | g5678820 | GenBank |
| | | | dominant) | | | | | | |
| 317 | 226305_at | | Homo sapiens mRNA; cDNA | | Consensussequence | Hs.6272.0 | AV696976 | Hs.6272.0.S1 | GenBank |
| | | | DKFZp761P0117 (from clone | | | | | | |
| | | | DKFZp761P0114) | | | | | | |
| 318 | 243692_at | GATA4 | GATA binding protein 4 | 8p23.1-p22 | Consensussequence | Hs.194114.0 | AW181962 | Hs.194114.0_RC | GenBank |
| 319 | 238460_at | | Homo sapiens cDNA FLJ25541 | | Consensussequence | Hs.272068.0 | AI590662 | Hs.272068.0 | GenBank |
| | | | fis, clone JTH00915. | | | | | | |
| 320 | 236812_at | | ESTs | | Consensussequence | Hs.223267.0 | AI638208 | Hs.223267.0.A1 | GenBank |
| 321 | 207284_s_at | ASPH | aspartate beta-hydroxylase | 8q12.1 | Exemplarsequence | Hs.283664.1 | NM_020164.1 | g9910363 | RefSeq |
| 322 | 237910_x_at | | ESTs | | Consensussequence | Hs.87672.1 | AI379467 | Hs.87672.1 | GenBank |
| 323 | 229430_at | MGC33510 | hypothetical protein MGC33510 | 8q12.3 | Consensussequence | Hs.184261.2 | AI421311 | Hs.184261.2_RC | GenBank |
| 324 | 235998_at | RHPN1 | rhophilin, Rho GTPase binding | 8q24.3 | Consensussequence | Hs.128081.0 | AI733369 | Hs.128081.0.A1 | GenBank |
| | | | protein 1 | | | | | | |
| 325 | 211148_s_at | ANGPT2 | angiopoietin 2 | 8p23.1 | Exemplarsequence | Hs.115181.1 | AF187858.1 | g8570646 | GenBank |
| 326 | 223843_at | SCARA3 | scavenger receptor class A, | 8p21 | Exemplarsequence | Hs.128856.1 | AB007830.1 | g6230378 | GenBank |
| | | | member 3 | | | | | | |

| # | Unigene_Accession | Cluster_Type | LocusLink | Full_Length_Reference_Seq |
|---|---|---|---|---|
| 1 | Hs.289063 | fulllength | 80346 | NM_025232; hypothetical protein FLJ22246 |
| 2 | Hs.380963 | fulllength | 29883 | NM_013354; CCR4-NOT transcription complex, subunit 7 isoform 1 NM_054026; |
| | | | | CCR4-NOT transcription complex, subunit 7 isoform 2 |
| 3 | Hs.7471 | fulllength | 83877 | NM_031940; BBP-like protein 1 isoform b NM_078473; BBP-like protein 1 isoform a |
| 4 | Hs.131255 | fulllength | 7381 | NM_006294; ubiquinol-cytochrome c reductase binding protein |
| 5 | Hs.167011 | | | |
| 6 | Hs.278634 | | 23514 | |
| 7 | Hs.288716 | fulllength | 80185 | NM_025115; hypothetical protein FLJ23263 |
| 8 | Hs.4147 | fulllength | 23471 | NM_014294; translocating chain-associating membrane protein |
| 9 | Hs.98471 | est | 137682 | NM_152416; hypothetical protein MGC40214 |
| 10 | Hs.170198 | fulllength | 9650 | NM_014637; KIAA0009 gene product |
| 11 | Hs.170198 | fulllength | 9650 | NM_014637; KIAA0009 gene product |
| 12 | Hs.243901 | fulllength | | |
| 13 | Hs.77100 | fulllength | 2961 | NM_002095; general transcription factor IIE, polypeptide 2, beta 34 kDa |
| 14 | Hs.27721 | fulllength | 54904 | NM_017778; WHSC1L1 protein isoform short NM_023034; WHSC1L1 protein |
| | | | | isoform long |
| 15 | Hs.169615 | fulllength | 65265 | NM_023080; hypothetical protein FLJ20989 |
| 16 | Hs.48876 | fulllength | 2222 | NM_004462; farnesyl-diphosphate farnesyltransferase 1 |
| 17 | Hs.356463 | fulllength | 2339 | NM_002027; farnesyltransferase, CAAX box, alpha |
| 18 | Hs.380963 | fulllength | 29883 | NM_013354; CCR4-NOT transcription complex, subunit 7 isoform 1 NM_054026; |
| | | | | CCR4-NOT transcription complex, subunit 7 isoform 2 |
| 19 | Hs.75737 | fulllength | 5108 | NM_006197; pericentriolar material 1 |
| 20 | Hs.75737 | fulllength | 5108 | NM_006197; pericentriolar material 1 |
| 21 | Hs.75737 | fulllength | 5108 | NM_006197; pericentriolar material 1 |
| 22 | Hs.27721 | fulllength | 54904 | NM_017778; WHSC1L1 protein isoform short NM_023034; WHSC1L1 protein |
| | | | | isoform long |
| 23 | Hs.55097 | fulllength | 28957 | NM_014018; mitochondrial ribosomal protein S28 |
| 24 | Hs.4742 | fulllength | 8733 | NM_003801; anchor attachment protein 1 |
| 25 | Hs.178551 | fulllength | 6132 | NM_000973; ribosomal protein L8 NM_033301; ribosomal protein L8 |
| 26 | Hs.10669 | fulllength | 50807 | |
| 27 | Hs.86970 | est | | |
| 28 | Hs.243901 | fulllength | | |
| 29 | Hs.33074 | | | |
| 30 | Hs.14158 | fulllength | 8895 | NM_003909; copine III |
| 31 | Hs.99519 | fulllength | 79752 | NM_024699; hypothetical protein FLJ14007 |
| 32 | Hs.94631 | fulllength | 10565 | NM_006421; brefeldin A-inhibited guanine nucleotide-exchange protein 1 |
| 33 | Hs.194562 | fulllength | 7013 | NM_003218; telomeric repeat binding factor 1 isoform 2 NM_017489; telomeric |
| | | | | repeat binding factor 1 isoform 1 |
| 34 | Hs.5333 | fulllength | 9920 | NM_014867; KIAA0711 gene product |
| 35 | Hs.75811 | fulllength | 427 | NM_004315; N-acylsphingosine amidohydrolase (acid ceramidase) 1 isoform b |
| | | | | NM_177924; N-acylsphingosine amidohydrolase (acid ceramidase) 1 preproprotein |
| | | | | isoform a |
| 36 | Hs.172816 | fulllength | 3084 | NM_004495; neuregulin 1 isoform HRG-gamma NM_013956; neuregulin 1 isoform |
| | | | | HRG-beta1 NM_013957; neuregulin 1 isoform HRG-beta2 NM_013958; neuregulin |
| | | | | 1 isoform HRG-beta3 NM_013959; neuregulin 1 isoform SMDF NM_013960; |
| | | | | neuregulin 1 isoform ndf43 NM_013961; neuregulin 1 isoform GGF NM_013962; |
| | | | | neuregulin 1 isoform GGF2 NM_013964; neuregulin 1 isoform HRG-alpha |
| 37 | Hs.193974 | fulllength | 2936 | NM_000637; glutathione reductase |
| 38 | Hs.7953 | fulllength | 51125 | NM_016099; HSPC041 protein |
| 39 | Hs.6856 | fulllength | 9070 | NM_004674; ash2 (absent, small, or homeotic)-like |
| 40 | Hs.72085 | fulllength | 55246 | NM_018246; hypothetical protein FLJ10853 |
| 41 | Hs.19673 | fulllength | 84232 | NM_032272; homolog of yeast MAF1 |
| 42 | Hs.339697 | fulllength | 51160 | NM_016208; VPS28 protein |
| 43 | Hs.11810 | fulllength | 83940 | NM_032026; CDA11 protein |
| 44 | Hs.9222 | fulllength | 9166 | NM_004215; estrogen receptor binding site associated antigen 9 |
| 45 | Hs.23528 | fulllength | 51123 | NM_016096; HSPC038 protein |
| 46 | Hs.77329 | fulllength | 9791 | NM_014754; phosphatidylserine synthase 1 |
| 47 | HS.416904 | | | |
| 48 | Hs.48876 | fulllength | 2222 | NM_004462; farnesyl-diphosphate farnesyltransferase 1 |
| 49 | Hs.356463 | fulllength | 2339 | NM_002027; farnesyltransferase, CAAX box, alpha |
| 50 | Hs.101617 | fulllength | 137492 | NM_152415; hypothetical protein FLJ32642 |
| 51 | Hs.101617 | fulllength | 137492 | NM_152415; hypothetical protein FLJ32642 |
| 52 | Hs.8294 | fulllength | 9897 | NM_014846; KIAA0196 gene product |
| 53 | Hs.75206 | fulllength | 5533 | NM_005605; protein phosphatase 3 (formerly 2B), catalytic subunit, gamma isoform |
| | | | | (calcineurin A gamma) |
| 54 | Hs.267905 | fulllength | 55140 | NM_018091; elongation protein 3 homolog |
| 55 | Hs.301959 | fulllength | 11212 | NM_007198; proline synthetase co-transcribed homolog |
| 56 | Hs.288057 | fulllength | 80140 | NM_025070; hypothetical protein FLJ22242 |
| 57 | Hs.86905 | fulllength | 528 | NM_001695; ATPase, H+ transporting, lysosomal 42 kD, V1 subunit C, isoform 1 |
| 58 | Hs.131255 | fulllength | 7381 | NM_006294; ubiquinol-cytochrome c reductase binding protein |
| 59 | Hs.44222 | fulllength | 51115 | NM_016033; CGI-90 protein |
| 60 | Hs.380969 | fulllength | 10987 | NM_006837; COP9 constitutive photomorphogenic homolog subunit 5 |
| 61 | Hs.48802 | fulllength | 66036 | NM_015458; myotubularin related protein 9 |
| 62 | Hs.15562 | fulllength | 55756 | NM_018250; hypothetical protein FLJ10871 |
| 63 | Hs.153678 | fulllength | 7993 | NM_005671; reproduction 8 |
| 64 | Hs.7381 | fulllength | 7419 | NM_005662; voltage-dependent anion channel 3 |
| 65 | Hs.334798 | fulllength | 1936 | NM_001960; eukaryotic translation elongation factor 1 delta isoform 2 NM_032378; |
| | | | | eukaryotic translation elongation factor 1 delta isoform 1 |
| 66 | Hs.12271 | fulllength | 26233 | NM_012162; F-box and leucine-rich repeat protein 6 isoform 1 NM_024555; F-box |
| | | | | and leucine-rich repeat protein 6 isoform 2 |
| 67 | Hs.4742 | fulllength | 8733 | NM_003801; anchor attachment protein 1 |
| 68 | Hs.339697 | fulllength | 51160 | NM_016208; VPS28 protein |
| 69 | Hs.376544 | | | |
| 70 | Hs.239784 | est | 23513 | NM_015356; scribble |
| 71 | Hs.404119 | fulllength | 7264 | NM_003313; tissue specific transplantation antigen P35B |
| 72 | Hs.318725 | fulllength | 51105 | NM_016018; CGI-72 protein |
| 73 | Hs.44159 | fulllength | 84165 | NM_032205; hypothetical protein FLJ21615 |
| 74 | Hs.318725 | fulllength | 51105 | NM_016018; CGI-72 protein |
| 75 | Hs.10669 | fulllength | 50807 | |
| 76 | Hs.10669 | fulllength | 50807 | |
| 77 | Hs.344478 | fulllength | 286053 | NM_173685; hypothetical protein FLJ32440 |
| 78 | Hs.95011 | fulllength | 6641 | NM_021021; basic beta 1 syntrophin |
| 79 | Hs.432544 | | | |
| 80 | Hs.283740 | fulllength | 56943 | NM_020189; DC6 protein |
| 81 | Hs.9222 | fulllength | 9166 | NM_004215; estrogen receptor binding site associated antigen 9 |
| 82 | Hs.169111 | fulllength | 55074 | NM_018002; oxidation resistance 1 |
| 83 | Hs.290880 | | | |
| 84 | Hs.351475 | fulllength | 5440 | NM_005034; DNA directed RNA polymerase II polypeptide K |
| 85 | Hs.197335 | fulllength | 10404 | NM_006102; NM_016134; plasma glutamate carboxypeptidase |
| 86 | Hs.16621 | fulllength | 25962 | NM_015496; DKFZP434I116 protein |
| 87 | Hs.279521 | fulllength | 55656 | NM_017864; hypothetical protein FLJ20530 |
| 88 | Hs.6390 | fulllength | | |
| 89 | Hs.6390 | fulllength | | |
| 90 | Hs.243901 | fulllength | | |
| 91 | Hs.29724 | fulllength | 79666 | NM_024613; phafin 2 |
| 92 | Hs.18426 | fulllength | 10247 | NM_005836; translational inhibitor protein p14.5 |
| 93 | Hs.131255 | fulllength | 7381 | NM_006294; ubiquinol-cytochrome c reductase binding protein |
| 94 | Hs.14158 | fulllength | 8895 | NM_003909; copine III |
| 95 | Hs.180612 | fulllength | 5828 | NM_000318; peroxisomal membrane protein 3 |
| 96 | Hs.25812 | fulllength | 4683 | NM_002485; nibrin |
| 97 | Hs.25812 | fulllength | 4683 | NM_002485; nibrin |
| 98 | Hs.96870 | fulllength | 27067 | NM_014393; staufen homolog 2 |
| 99 | Hs.106650 | fulllength | 54968 | NM_017866; hypothetical protein FLJ20533 |
| 100 | Hs.4147 | fulllength | 23471 | NM_014294; translocating chain-associating membrane protein |
| 101 | Hs.94631 | fulllength | 10565 | NM_006421; brefeldin A-inhibited guanine nucleotide-exchange protein 1 |
| 102 | Hs.94631 | fulllength | 10565 | NM_006421; brefeldin A-inhibited guanine nucleotide-exchange protein 1 |
| 103 | Hs.380877 | fulllength | 23678 | NM_013257; serum/glucocorticoid regulated kinase-like isoform 1 NM_170709; |
| | | | | serum/glucocorticoid regulated kinase-like isoform 2 |
| 104 | Hs.380877 | fulllength | 23678 | NM_013257; serum/glucocorticoid regulated kinase-like isoform 1 NM_170709; |
| | | | | serum/glucocorticoid regulated kinase-like isoform 2 |
| 105 | Hs.106768 | fulllength | 55156 | NM_018120; armadillo repeat-containing protein |
| 106 | Hs.283664 | fulllength | 444 | NM_004318; aspartate beta-hydroxylase isoform a NM_020164; aspartate beta- |
| | | | | hydroxylase isoform e NM_032466; aspartate beta-hydroxylase isoform c |
| | | | | NM_032467; aspartate beta-hydroxylase isoform d NM_032468; aspartate beta- |
| | | | | hydroxylase isoform b |
| 107 | Hs.78305 | fulllength | 5862 | NM_002865; RAB2, member RAS oncogene family |
| 108 | Hs.78305 | fulllength | 5862 | NM_002865; RAB2, member RAS oncogene family |
| 109 | Hs.323164 | fulllength | 79145 | NM_024300; hypothetical protein MGC2217 |
| 110 | Hs.79300 | fulllength | 7336 | NM_003350; ubiquitin-conjugating enzyme E2 variant 2 |
| 111 | Hs.26458 | fulllength | 4482 | NM_012331; methionine sulfoxide reductase A |
| 112 | Hs.19575 | fulllength | 51606 | NM_015941; ATPase, H+ transporting, lysosomal 50/57 kDa, V1 subunit H |
| 113 | Hs.173381 | fulllength | 1808 | NM_001386; dihydropyrimidinase-like 2 |
| 114 | Hs.300861 | | | |
| 115 | Hs.267905 | fulllength | 55140 | NM_018091; elongation protein 3 homolog |
| 116 | Hs.193974 | fulllength | 2936 | NM_000637; glutathione reductase |
| 117 | Hs.137732 | fulllength | 23087 | NM_015066; tripartite motif-containing 35 isoform 1 NM_171982; tripartite motif- |
| | | | | containing 35 isoform 2 |
| 118 | Hs.274136 | fulllength | 55290 | NM_018310; RNA polymerase III transcription initiation factor BRF2 |
| 119 | Hs.170822 | fulllength | 81790 | NM_030954; hypothetical protein DKFZp564A022 |
| 120 | Hs.355753 | | 137964 | NM_178819; putative lysophosphatidic acid acyltransferase |
| 121 | Hs.125849 | fulllength | 11160 | NM_007175; chromosome 8 open reading frame 2 |
| 122 | Hs.125849 | fulllength | 11160 | NM_007175; chromosome 8 open reading frame 2 |
| 123 | Hs.226573 | fulllength | 3551 | |
| 124 | Hs.301959 | fulllength | 11212 | NM_007198; proline synthetase co-transcribed homolog |
| 125 | Hs.128856 | fulllength | 51435 | NM_016240; CSR1 protein |
| 126 | Hs.61696 | | | |
| 127 | Hs.443139 | est | | |
| 128 | Hs.18029 | fulllength | 55093 | NM_018024; hypothetical protein FLJ10204 |
| 129 | Hs.179909 | fulllength | 96764 | NM_024831; PRIP-interacting protein PIPMT |
| 130 | Hs.377879 | fulllength | 157697 | |
| 131 | Hs.299148 | fulllength | 64760 | NM_022749; retinoic acid induced 16 |
| 132 | Hs.33787 | fulllength | 10174 | NM_005775; vinexin bets SH3-containing adaptor molecule-1) |
| 133 | | | | |
| 134 | Hs.196270 | fulllength | 81034 | NM_030780; mitochondrial folate transporter/carrier |
| 135 | Hs.180612 | fulllength | 5828 | NM_000318; peroxisomal membrane protein 3 |
| 136 | Hs.93005 | fulllength | 6591 | NM_003068; snail 2 |
| 137 | Hs.273344 | fulllength | 25879 | NM_014158; NM_015420; DKFZP564O0463 protein |
| 138 | Hs.425311 | fulllength | 27257 | NM_014462; Lsm1 protein |
| 139 | Hs.28285 | fulllength | 11236 | NM_007218; ring finger protein 139 |
| 140 | Hs.51233 | fulllength | 8795 | NM_003842; tumor necrosis factor receptor superfamily, member 10b isoform 1 |
| | | | | precursor NM_147187; tumor necrosis factor receptor superfamily, member 10b |
| | | | | isoform 2 precursor |
| 141 | Hs.26102 | fulllength | 7227 | NM_014112; zinc finger transcription factor TRPS1 |
| 142 | Hs.279704 | fulllength | 54108 | NM_017444; chromatin accessibility complex 1 |
| 143 | Hs.300224 | fulllength | 51236 | NM_016458; brain protein 16 |
| 144 | Hs.333388 | fulllength | 93100 | NM_145201; similar to CG3714 gene product |
| 145 | Hs.84087 | | 23167 | |
| 146 | Hs.84087 | | 23167 | |
| 147 | Hs.26102 | fulllength | 7227 | NM_014112; zinc finger transcription factor TRPS1 |
| 148 | Hs.128645 | fulllength | 64089 | NM_022133; sorting nexin 16 isoform a NM_152836; sorting nexin 16 isoform a |
| | | | | NM_152837; sorting nexin 16 isoform b |
| 149 | Hs.266175 | fulllength | 55824 | NM_018440; phosphoprotein associated with glycosphingolipid-enriched |
| | | | | microdomains |
| 150 | Hs.184693 | fulllength | 6921 | NM_005648; elongin C |
| 151 | Hs.179574 | fulllength | 5520 | NM_002717; protein phosphatase 2 (formerly 2A), regulatory subunit B (PR 52), |
| | | | | alpha isoform |
| 152 | Hs.124961 | est | | |
| 153 | Hs.173159 | fulllength | 6867 | NM_006283; transforming, acidic coiled-coil containing protein 1 |
| 154 | Hs.380935 | fulllengh | 3297 | NM_005526; heat shock transcription factor 1 |
| 155 | Hs.74562 | fulllength | 22827 | NM_014281; fuse-binding protein-interacting repressor isoform b NM_078480; fuse- |
| | | | | binding protein-interacting repressor isoform a |
| 156 | Hs.83727 | fulllength | 29894 | NM_013291; cleavage and polyadenylation specific factor 1, 160 kDa |
| 157 | Hs.27842 | fulllength | 55326 | NM_018361; acid acyltransferase-epsilon |
| 158 | Hs.71215 | fulllength | 9046 | NM_003974; docking protein 2, 56 kD |
| 159 | Hs.287624 | fulllength | 80094 | NM_025026; hypothetical protein FLJ14107 |
| 160 | Hs.6459 | fulllength | 79581 | NM_024531; putative G-protein coupled receptor GPCR41 |
| 161 | Hs.323114 | fulllength | 79139 | NM_018630; NM_024295; hypothetical protein MGC3067 |
| 162 | Hs.58189 | fulllength | 8667 | NM_003756; eukaryotic translation initiation factor 3, subunit 3 gamma, 40 kDa |
| 163 | Hs.283740 | fulllength | 56943 | NM_020189; DC6 protein |
| 164 | Hs.106673 | fulllength | 3646 | NM_001568; murine mammary tumor integration site 6 (oncogene homolog) |
| 165 | Hs.197335 | fulllength | 10404 | NM_006102; NM_016134; plasma glutamate carboxypeptidase |
| 166 | Hs.171776 | fulllength | 3612 | NM_005536; inositol(myo)-1(or 4)-monophosphetase 1 |
| 167 | Hs.287727 | fulllength | 80124 | NM_025054; valosin-containing protein (p97)/p47 complex-interacting protein p135 |
| 168 | Hs.5518 | fulllength | 253943 | NM_152758; hypothetical protein FLJ31657 |
| 169 | Hs.12540 | fulllength | 10434 | NM_006330; lysophospholipase I |
| 170 | Hs.127675 | fulllength | 2055 | NM_018941; CLN8 protein |
| 171 | Hs.75811 | fulllength | 427 | NM_004315; N-acylsphingosine amidohydrolase (acid ceramidase) 1 isoform b |
| | | | | NM_177924; N-acylsphingosine amidohydrolase (acid ceramidase) 1 preproprotein |
| | | | | isoform a |
| 172 | Hs.77135 | fulllength | 84549 | NM_032509; RNA binding protein |
| 173 | Hs.279921 | fulllength | 51669 | NM_016127; hypothetical protein MGC8721 |
| 174 | Hs.151237 | fulllength | 79845 | NM_024787; hypothetical protein FLJ12526 |
| 175 | Hs.194726 | fulllength | 9530 | NM_004874; BCL2-associated athanogene 4 |
| 176 | Hs.7432 | fulllength | 55145 | NM_018105; hypothetical protein FLJ10477 |
| 177 | Hs.2076 | fulllength | 7553 | NM_003416; zinc finger protein 7 (KOX 4, clone HF.16) |
| 178 | Hs.289271 | fulllength | 1537 | NM_001916; cytochrome c-1 |
| 179 | Hs.122752 | fulllength | 6873 | NM_003184; TBP-associated factor 2 |
| 180 | Hs.81848 | fulllength | 5885 | NM_006265; RAD21 homolog |
| 181 | Hs.166684 | fulllength | 6788 | NM_006281; serine/threonine kinase 3(STE20 homolog, yeast) |
| 182 | Hs.278428 | fulllength | 51366 | NM_015902; progestin induced protein |
| 183 | Hs.355977 | fulllength | 11059 | NM_007013; WW domain-containing protein 1 |
| 184 | Hs.18349 | fulllength | 29088 | NM_014175; mitochondrial ribosomal protein L15 |
| 185 | Hs.356463 | fulllength | 2339 | NM_002027; famesyltransferase, CAAX box, alpha |
| 186 | Hs.1697 | fulllength | 526 | NM_001693; ATPase, H+ transporting, lysosomal 56/56 kD, V1 subunit B, isoform 2 |
| 187 | Hs.75206 | fulllength | 5533 | NM_005605; protein phosphatese 3 (formerly 2B), catalytic subunit, gamma isoform |
| | | | | calcineurin A gamma) |
| 188 | Hs.15711 | fulllength | 23303 | NM_015254; kinesin family member 13B |
| 189 | Hs.80350 | fulllength | 5516 | NM_004156; protein phoshatase 2 (formerly 2A), catalytic subunit, beta isoform |
| 190 | Hs.433317 | fulllength | 1978 | NM_004095; eukaryotic translation initiation factor 4E binding protein 1 |
| 191 | Hs.26450 | fulllength | 23259 | |
| 192 | Hs.10018 | fulllength | 114926 | NM_138436; hypothetical protein BC013035 |
| 193 | Hs.180107 | fulllength | 5423 | NM_002690; polymerase (DNA directed), beta |
| 194 | Hs.27721 | fulllength | 54904 | NM_017778; WHSC1L1 protein isoform short NM_023034; WHSC1L1 protein |
| | | | | isoform long |
| 195 | Hs.75789 | fulllength | 10397 | NM_006096; N-myc downstream regulated gene 1 |
| 196 | Hs.351875 | fulllength | 1345 | NM_004374; cytochrome c oxidase subunit Vic proprotein |
| 197 | Hs.351475 | fulllength | 5440 | NM_005034; DNA directed RNA polymerase II polypeptide K |
| 198 | Hs.81548 | fulllength | 1666 | NM_001359; 2,4-dienoyl CoA reductase 1 precursor |
| 199 | Hs.154443 | fulllength | 4173 | |
| 200 | Hs.51233 | fulllength | 8795 | NM_003842; tumor necrosis factor receptor superfamily, member 10b isoform 1 |
| | | | | precursor NM_147187; tumor necrosis factor receptor superfamily, member 10b |
| | | | | isoform 2 precursor |
| 201 | Hs.71119 | fulllength | 7991 | NM_006765; Putative prostate cancer tumor suppressor isoform a NM_178234; |
| | | | | Putative prostate cancer tumor suppressor isoform b |
| 202 | Hs.288627 | fulllength | 8694 | NM_012079; diacylglycerol O-acyltransferase 1 |
| 203 | Hs.348609 | fulllength | 84875 | NM_032789; hypothetical protein FLJ14464 |
| 204 | Hs.4742 | fulllength | 8733 | NM_003801; anchor attachment protein 1 |
| 205 | Hs.18166 | | 22898 | |
| 206 | Hs.352415 | fulllength | 55630 | NM_017767; solute carrier family 39 (zinc transporter), member 4 NM_130849; |
| | | | | solute carrier family 39 (zinc transporter), member 4 |
| 207 | Hs.157240 | fulllength | 83696 | NM_031466; hypothetical protein MGC4737 |
| 208 | Hs.83727 | fulllength | 29894 | NM_013291; cleavage and polyadenylation specific factor 1, 160 kDa |
| 209 | Hs.44159 | fulllength | 84165 | NM_032205; hypothetical protein FLJ21615 |
| 210 | Hs.318725 | fulllength | 51105 | NM_016018; CGI-72 protein |
| 211 | Hs.301698 | fulllength | 6482 | NM_003033; sialyltransferase 4A NM_173344; sialyltransferase 4A |
| 212 | Hs.92918 | fulllength | 51571 | NM_016623; hypothetical protein BM-009 |
| 213 | Hs.10669 | fulllength | 50807 | |
| 214 | Hs.10669 | fulllength | 50807 | |
| 215 | Hs.133244 | fulllength | 56169 | NM_031415; melanoma-derived leucine zipper, extra-nuclear factor |
| 216 | Hs.7837 | fulllength | 10221 | NM_025195; G-protein-coupled receptor induced protein |
| 217 | Hs.12940 | fulllength | 11244 | NM_007222; zinc finger and homeodomain protein 1 NM_032847; hypothetical |
| | | | | protein FLJ14825 |
| 218 | Hs.241576 | fulllength | 55493 | |
| 219 | Hs.222088 | fulllength | 29028 | NM_014109; PRO2000 protein NM_032365; |
| 220 | Hs.12940 | fulllength | 11244 | NM_007222; zinc finger and homeodomain protein 1 |
| 221 | Hs.26102 | fulllength | 7227 | NM_014112; zinc finger transcription factor TRPS1 |
| 222 | Hs.26102 | fulllength | 7227 | NM_014112; zinc finger transcription factor TRPS1 |
| 223 | Hs.298229 | fulllength | 5202 | NM_012394; prefoldin 2 NM_080651; TRAP/Mediator complex component |
| | | | | TRAP25 |
| 224 | Hs.235935 | fulllength | 4856 | NM_002514; nov precursor |
| 225 | Hs.235935 | fulllength | 4856 | NM_002514; nov precursor |
| 226 | Hs.81848 | fulllength | 5885 | NM_006265; RAD21 homolog |
| 227 | Hs.283740 | fulllength | 56943 | NM_020189; DC6 protein |
| 228 | Hs.94262 | fulllength | 50484 | |
| 229 | Hs.75103 | fulllength | 7534 | NM_003406; tyrosine 3/tryptophan 5-monooxygenase activation protein, zeta |
| | | | | polypeptide NM_145690; tyrosine 3/tryptophan 5-monooxygenase activation |
| | | | | protein, zeta polypeptide |
| 230 | Hs.75103 | fulllength | 7534 | NM_003406; tyrosine 3/tryptophan 5-monooxygenase activation protein, zeta |
| | | | | polypeptide NM_145690; tyrosine 3/tryptophan 5-monooxygenase activation |
| | | | | protein, zeta polypeptide |
| 231 | Hs.75103 | fulllength | 7534 | NM_003406; tyrosine 3/tryptophan 5-monooxygenase activation protein, zeta |
| | | | | polypeptide NM_145690; tyrosine 3/tryptophan 5-monooxygenase activation |
| | | | | protein, zeta polypeptide |
| 232 | Hs.75103 | fulllength | 7534 | NM_003406; tyrosine 3/tryptophan 5-monooxygenase activation protein, zeta |
| | | | | polypeptide NM_145690; tyrosine 3/tryptophan 5-monooxygenase activation |
| | | | | protein, zeta polypeptide |
| 233 | Hs.405590 | est | | |
| 234 | Hs.75367 | fulllength | 6503 | NM_006748; Src-like-adaptor |
| 235 | Hs.307057 | fulllength | 83955 | NM_032031; FKSG17 |
| 236 | Hs.223014 | fulllength | 51582 | NM_015878; ornithine decarboxylase antizyme inhibitor NM_148174; ornithine |
| | | | | decarboxylase antizyme inhibitor |
| 237 | Hs.223014 | fulllength | 51582 | NM_015878; ornithine decarboxylase antizyme inhibitor NM_148174; ornithine |
| | | | | decarboxylase antizyme inhibitor |
| 238 | Hs.153057 | fulllength | 6674 | NM_003114; sperm associated antigen 1 NM_172218; sperm associated antigen 1 |
| 239 | Hs.82173 | fulllength | 7071 | NM_005655; TGFB inducible early growth response |
| 240 | Hs.333823 | fulllength | 28998 | NM_014078; mitochondrial ribosomal protein L13 |
| 241 | Hs.46680 | fulllength | 51001 | NM_015942: CGI-12 protein |
| 242 | Hs.29724 | fulllength | 79666 | NM_024613; phafin 2 |
| 243 | Hs.278428 | fulllength | 51366 | NM_015902; progestin induced protein |
| 244 | Hs.21621 | fulllength | 55529 | NM_018710; hypothetical protein DKFZp762O076 |
| 245 | Hs.266175 | fulllength | 55824 | NM_018440; phosphoprotein associated with glycosphingolipid-enriched |
| | | | | microdomains |
| 246 | Hs.13256 | | | |
| 247 | Hs.103755 | fulllength | 8767 | NM_003821; receptor-interacting serine-threonine kinase 2 |
| 248 | Hs.103755 | fulllength | 8767 | NM_003821; receptor-interacting serine-threonine kinase 2 |
| 249 | Hs.355977 | fulllength | 11059 | NM_007013; WW domain-containing protein 1 |
| 250 | Hs.237146 | fulllength | 65986 | NM_023929; zinc finger protein RINZF |
| 251 | Hs.21275 | fulllength | 55284 | NM_018299; hypothetical protein FLJ11011 |
| 252 | Hs.21275 | fulllength | 55284 | NM_018299; hypothetical protein FLJ11011 |
| 253 | Hs.15977 | fulllength | 4715 | NM_005005; NADH dehydrogenase (ubiquinone) 1 beta subcomplex, 9, 22 kDa |
| 254 | Hs.184693 | fulllength | 6921 | NM_005648; elongin C |
| 255 | Hs.404048 | fulllength | 10499 | NM_006540; nuclear receptor coactivator 2 |
| 256 | Hs.404048 | fulllength | 10499 | NM_006540; nuclear receptor coactivator 2 |
| 257 | Hs.296730 | fulllength | 80243 | NM_025170; hypothetical protein FLJ12987 |
| 258 | Hs.80887 | fulllength | 4067 | NM_002350; v-yes-1 Yamaguchi sarcoma viral related oncogene homolog |
| 259 | Hs.80887 | fulllength | 4067 | NM_002350; v-yes-1 Yamaguchi sarcoma viral related oncogene homolog |
| 260 | Hs.80887 | fulllength | 4067 | NM_002350; v-yes-1 Yamaguchi sarcoma viral related oncogene homolog |
| 261 | Hs.194562 | fulllength | 7013 | NM_003218; telomeric repeat binding factor 1 isoform 2 NM_017489; telomeric |
| | | | | repeat binding factor 1 isoform 1 |
| 262 | Hs.78619 | fulllength | 8836 | NM_003878: gamma-glutamyl hydrolase precursor |
| 263 | Hs.283664 | fulllength | 444 | NM_004318; aspartate beta-hydroxylase isoform a NM_020164; aspartale beta- |
| | | | | hydroxylase isoform e NM_032466; aspartate beta-hydroxylase isoform c |
| | | | | NM_032467; aspartate beta-hydroxylase isoform d NM_032468; aspartate beta- |
| | | | | hydroxylase isoform b |
| 264 | Hs.105461 | | 55636 | |
| 265 | Hs.155572 | | 137886 | |
| 266 | Hs.78305 | fulllength | 5862 | NM_002865; RAB2, member RAS oncogene family |
| 267 | Hs.78305 | fulllength | 5862 | NM_002865; RAB2, member RAS oncogene family |
| 268 | Hs.78305 | fulllength | 5862 | NM_002865; RAB2, member RAS oncogene family |
| 269 | Hs.14968 | fulllength | 5324 | NM_002655; pleiomorphic adenoma gene 1 |
| 270 | Hs.8102 | fulllength | 6224 | NM_001023; ribosomal protein S20 |
| 271 | Hs.8180 | fulllength | 6386 | NM_005625; syndecan binding protein (syntenin) |
| 272 | Hs.378857 | fulllength | 54928 | NM_017813; hypothetical protein FLJ20421 |
| 273 | Hs.12540 | fulllength | 10434 | NM_006330; lysophospholipase I |
| 274 | Hs.323164 | fulllength | 79145 | NM_024300; hypothetical protein MGC2217 |
| 275 | Hs.76722 | fulllength | 1052 | NM_005195; CCAAT/enhancer binding protein delta |
| 276 | Hs.154443 | fulllength | 4173 | |
| 277 | Hs.155637 | fulllength | 5591 | NM_006904; protein kinase, DNA-activated, catalytic polypeptide |
| 278 | Hs.155637 | fulllength | 5591 | NM_006904; protein kinase, DNA-activated, catalytic polypeptide |
| 279 | Hs.81001 | fulllength | 26260 | NM_012173; F-box only protein 25 |
| 280 | Hs.155958 | fulllength | 9 | NM_000662 N-acetyltransferase 1 |
| 281 | Hs.180178 | fulllength | 91694 | NM_152271; hypothetical protein FLJ23749 |
| 282 | Hs.75811 | fulllength | 427 | NM_004315; N-acylsphingosine amidohydrolase (acid ceramidase) 1 isoform b |
| | | | | NM_177924; N-acylsphingosine amidohydrolase (acid ceramidase) 1 preproprotein |
| | | | | isoform a |
| 283 | Hs.155272 | est | | |
| 284 | Hs.297939 | fulllength | 1508 | NM_001908; cathepsin B preproprotein NM_147780; cathepsin B preproprotein |
| | | | | NM_147781; cathepsin B preproprotein NM_147782; cathepsin B preproprotein |
| | | | | NM_147783; cathepsin B preproprotein |
| 285 | Hs.297939 | fulllength | 1508 | NM_001908; cathepsin B preproprotein NM_147780; cathepsin B preproprotein |
| | | | | NM_147781; cathepsin B preproprotein NM_147782; cathepsin B preproprotein |
| | | | | NM_147783; cathepsin B preproprotein |
| 286 | Hs.297939 | fulllength | 1508 | NM_001908; cathepsin B preproprotein NM_147780; cathepsin B preproprotein |
| | | | | NM_147781; cathepsin B preproprotein NM_147782; cathepsin B preproprotein |
| | | | | NM_147783; cathepsin B preproprotein |
| 287 | Hs.25590 | fulllength | 6781 | NM_003155; stanniocalcin 1 |
| 288 | Hs.11260 | fulllength | 55790 | NM_018371; chondroitin beta1,4 N-acetylgalactosaminyltransferase |
| 289 | Hs.131056 | | | |
| 290 | Hs.300861 | | | |
| 291 | Hs.201377 | fulllength | 9583 | NM_004901; lysosomal apyrase-like 1 |
| 292 | Hs.379109 | fulllength | 64236 | NM_021630; PDZ and LIM domain 2 NM_176871; PDZ and LIM domain 2 |
| 293 | Hs.72071 | fulllength | 54793 | NM_017634; hypothetical protein FLJ20038 |
| 294 | Hs.20313 | fulllength | 2185 | NM_004103; PTK2B protein tyrosine kinase 2 beta isoform a NM_173174; PTK2B |
| | | | | protein tyrosine kinase 2 beta isoform a NM_173175; PTK2B protein tyrosine |
| | | | | kinase 2 beta isoform b NM_173176; PTK2B protein tyrosine kinase 2 beta isoform a |
| 295 | Hs.39913 | fulllength | 10671 | NM_006571; dynactin 6 |
| 296 | Hs.17270 | fulllength | 25960 | NM_032777; G protein-coupled receptor 124 |
| 297 | Hs.2442 | fulllength | 8754 | NM_003816; a disintegrin and metalloproteinase domain 9 preproprotein |
| 298 | Hs.18585 | | | |
| 299 | Hs.125849 | fulllength | 11160 | NM_007175; chromosome 8 open reading frame 2 |
| 300 | Hs.82210 | fulllength | 7994 | NM_006766; MYST histone acetyltransferase (monocytic leukemia) 3 |
| 301 | Hs.173159 | fulllength | 6867 | NM_006283; transforming, acidic coiled-coil containing protein 1 |
| 302 | Hs.359201 | est | | |
| 303 | Hs.2331 | fulllength | 1875 | NM_001951; E2F transcription factor 5 |
| 304 | Hs.25590 | fulllength | 6781 | NM_003155; stanniocalcin 1 |
| 305 | Hs.41185 | fulllength | 157869 | NM_153225; hypothetical protein FLJ40021 |
| 306 | Hs.108854 | fulllength | 29097 | NM_014184; HSPC163 protein |
| 307 | Hs.272367 | fulllength | 55806 | NM_005144; hairless protein isoform a NM_018411; hairless protein isoform b |
| 308 | Hs.1274 | fulllength | 649 | NM_001199; bone morphogenetic protein 1 isoform 1, precursor NM_006128; bone |
| | | | | morphogenetic protein 1 isoform 2, precursor NM_006129; bone morphogenetic |
| | | | | protein 1 isoform 3, precursor NM_006130; bone morphogenetic protein 1 isoform 6 |
| | | | | precursor NM_006131; bone morphogenetic protein 1 isoform 5, precursor |
| | | | | NM_006132; bone morphogenetic protein 1 isoform 4, precursor |
| 309 | Hs.149152 | fulllength | 114822 | NM_052924; rhophilin 1 |
| 310 | Hs.274136 | fulllength | 55290 | NM_018310; RNA polymerase III transcription initiation factor BRF2 |
| 311 | Hs.17270 | fulllength | 25960 | NM_032777; G protein-coupled receptor 124 |
| 312 | Hs.103849 | fulllength | 84257 | NM_032297; hypothetical protein DKFZp761D112 |
| 313 | Hs.165741 | fulllength | 56260 | NM_019607; hypothetical protein FLJ11267 |
| 314 | Hs.133546 | fulllength | 79824 | |
| 315 | Hs.151449 | fulllength | 9705 | NM_014682; suppression of tumorigenicity 18 |
| 316 | Hs.251687 | fulllength | 6101 | NM_006269; retinitis pigmentosa RP1 protein |
| 317 | Hs.6272 | | | |
| 318 | Hs.243987 | fulllength | 2626 | NM_002052; GATA binding protein 4 |
| 319 | Hs.379094 | | | |
| 320 | Hs.201058 | est | | |
| 321 | Hs.283664 | fulllength | 444 | NM_004318; aspartate beta-hydroxylase isoform a NM_020164; aspartate beta- |
| | | | | hydroxylase isoform e NM_032466; aspartate beta-hydroxylase isoform c |
| | | | | NM_032467; aspartate beta-hydroxylase isoform d NM_032468; aspartate beta- |
| | | | | hydroxylase isoform b |
| 322 | Hs.445714 | est | | |
| 323 | Hs.184261 | fulllength | 254778 | NM_152765; hypothetical protein MGC33510 |
| 324 | Hs.149152 | fulllength | 114822 | NM_052924; rhophilin 1 |
| 325 | Hs.115181 | fulllength | 285 | NM_001147; angiopoietin 2 |
| 326 | Hs.128856 | fulllength | 51435 | NM_016240; CSR1 protein |

TABLE 31
_other

| | | | | | | | | | | Sequence Derived |
|---|---|---|---|---|---|---|---|---|---|---|
| # | affy id | HUGO name | F | p | q | Title | MapLocation | Sequence Type | Transcript ID | From |
| 1 | 230024_at | | 71.32 | 6.16e−12 | 2.37e−07 | ESTs | | Consensussequence | Hs.143698.0 | AI183453 |
| 2 | 206207_at | CLC | 42.57 | 5.45e−09 | 1.05e−04 | Charot-Leyden crystal protein | 19q13.1 | Exemplarsequence | Hs.889.0 | NM_001828.3 |
| 3 | 200923_at | LGALS3BP | 34.29 | 8.73e−08 | 1.03e−03 | lectin, galactoside-binding, | 17q25 | Exemplarsequence | Hs.79339.0 | NM_005567.2 |
| | | | | | | soluble, 3 binding protein | | | | |
| 4 | 223708_at | C1QTNF4 | 33.73 | 1.07e−07 | 1.03e−03 | C1q and tumor necrosis factor related protein 4 | 11q11 | Exemplarsequence | Hs.119302.0 | AF329838.1 |
| 5 | 243577_at | | 33.33 | 7.32e−07 | 4.02e−03 | ESTs | | Consensussequence | Hs.146144.0 | AA643238 |
| 6 | 204116_at | IL2RG | 33.19 | 1.75e−07 | 1.35e−03 | interleukin 2 receptor, gamma | Xq13.1 | Exemplarsequence | Hs.84.0 | NM_000206.1 |
| | | | | | | (severe combined | | | | |
| | | | | | | immunodeficiency) | | | | |
| 7 | 233452_at | | 32.24 | 5.60e−07 | 3.58e−03 | | | Consensussequence | Hs.283931.0 | AF150222 |
| 8 | 238502_at | | 29.34 | 1.04e−06 | 4.98e−03 | ESTs | | Consensussequence | Hs.137190.0 | BE886165 |
| 9 | 209194_at | CETN2 | 38195 | 4.96e−05 | 7.16e−02 | centrin, EF-hand protein, 2 | Xq28 | Exemplarsequence | Hs.82794.0 | BC005334.1 |
| 10 | 239742_at | | 38103 | 2.02e−06 | 8.63e−03 | Homo sapiens full length insert cDNA clone YU79H10 | | Consensussequence | Hs.31319.0 | H15278 |
| 11 | 228904_at | | 25.62 | 3.37e−06 | 1.29e−02 | ESTS | | Consensussequence | Hs.156044.0 | AW510657 |
| 12 | 211743_s_at | PRG2 | 25.27 | 4.54e−06 | 1.53e−02 | proteoglycan 2, bone marrow | 11q12 | Exemplarsequence | g13543541 | BC005929.1 |
| | | | | | | (natural killer cell activator, | | | | |
| | | | | | | eosinophil granule major basic protein) | | | | |
| 13 | 214450_at | CTSW | 25.19 | 5.18e−06 | 1.53e−02 | cathepsin W (lymphopain) | 11q13.1 | Consensussequence | Hs.87450.0 | NM_001335.1 |
| 14 | 205297_s_at | CD79B | 38071 | 2.19e−05 | 4.67e−02 | CD79B antigen (immunoglobulin- | 17q23 | Exemplarsequence | Hs.89575.1 | NM_000626.1 |
| | | | | | | associated beta) | | | | |
| 15 | 226670_s_at | C20orf119 | 24.48 | 8.88e−06 | 2.27e−02 | chromosome 20 open reading | | Consensussequence | Hs.251946.0 | AL109839 |
| | | | | | | frame 119 | | | | |
| 16 | 214836_x_at | IGKC | 24.30 | 4.95e−06 | 1.53e−02 | immunoglobulin kappa constant | 2p12 | Consensussequence | Hs.123030.0 | BG536224 |
| 17 | 239105_at | | 23.82 | 6.34e−05 | 7.16e−02 | ESTs | | Consensussequence | Hs.121525.0 | AI681581 |
| 18 | 214627_at | EPX | 23.59 | 5.73e−06 | 1.57e−02 | eosinophil peroxidase | 17q23.1 | Consensussequence | Hs.46295.0 | X14346.1 |
| 19 | 243021_at | | 22.75 | 1.20e−05 | 2.88e−02 | ESTs, Weakly similar to CGHU7L | | Consensussequence | Hs.148652.1 | AI344101 |
| | | | | | | collagen alpha 1(III) chain | | | | |
| | | | | | | precursor - human [H. sapiens] | | | | |
| 20 | 230670_at | FLJ25972 | 22.47 | 1.98e−05 | 4.48e−02 | hypothetical protein FLJ25972 | 3q25.1 | Consensussequence | Hs.88162.0 | AW341661 |
| 21 | 202044_at | GRLF1 | 22.38 | 5.51e−04 | 0.24 | glucocorticoid receptor DNA | 19q13.3 | Consensussequence | Hs.102548.0 | NM_004491.1 |
| | | | | | | binding factor 1 | | | | |
| 22 | 213665_at | SOX4 | 21.29 | 7.57e−05 | 7.46e−02 | SRY (sex determining region Y)- | 6p22.3 | Consensussequence | Hs.83484.1 | AI989477 |
| | | | | | | box 4 | | | | |
| 23 | 219734_at | FLJ20174 | 21.27 | 4.99e−05 | 7.16e−02 | hypothetical protein FLJ20174 | 3q13.2 | Exemplarsequence | Hs.114556.0 | NM_017699.1 |
| 24 | 244043_at | | 21.21 | 1.46e−04 | 0.11 | ESTs, Weakly similar to | | Consensussequence | Hs.283390.0 | AI049624 |
| | | | | | | hypothetical protein FLJ20378 | | | | |
| | | | | | | [Homo sapiens] [H. sapiens] | | | | |
| 25 | 221782_at | ERdj5 | 20.64 | 1.31e−04 | 0.10 | ER-resident protein ERdj5 | 2q32.1 | Consensussequence | Hs.1098.0 | AL137648.1 |
| 26 | 244166_at | | 20.60 | 1.17e−04 | 9.74e−02 | ESTs | | Consensussequence | Hs.248476.0 | AI762422 |
| 27 | 213398_s_at | KIAA0323 | 19.99 | 1.53e−04 | 0.12 | KIAA0323 protein | 14q11.2 | Consensussequence | Hs.7911.1 | AI347090 |
| 28 | 237422_at | | 19.89 | 2.49e−05 | 5.04e−02 | ESTs | | Consensussequence | Hs.190440.0 | AA873726 |
| 29 | 209757_s_at | MYCN | 19.89 | 3.11e−05 | 5.70e−02 | v-myc myelocytomatosis viral | 2p24.1 | Exemplarsequence | Hs.25960.1 | BC002712.1 |
| | | | | | | related oncogene, neuroblastoma | | | | |
| | | | | | | derived (avian) | | | | |
| 30 | 236173_s_at | LRIG1 | 19.66 | 2.83e−05 | 5.43e−02 | leucine-rich repeats and | | Consensussequence | Hs.121706.0 | AA775396 |
| | | | | | | immunoglobulin-like domains 1 | | | | |
| 31 | 243010_at | MSI2 | 19.56 | 5.75e−05 | 7.16e−02 | musashi homolog 2 (Drosophila) | 17q23.1 | Consensussequence | Hs.103512.0 | BE000929 |
| 32 | 223723_at | MFI2 | 19.28 | 6.65e−05 | 7.16e−02 | antigen p97 (melanoma | 3q28-q29 | Exemplarsequence | Hs.271966.0 | BC001875.1 |
| | | | | | | associated) identified by | | | | |
| | | | | | | monoclonal antibodies 133.2 and | | | | |
| | | | | | | 96.5 | | | | |
| 33 | 206998_x_at | PRB3 | 19.22 | 5.85e−05 | 7.16e−02 | proline-rich protein BstNI | 2p13.2 | Exemplarsequence | Hs.73031.0 | NM_006249.2 |
| | | | | | | subfamily 3 | | | | |
| 34 | 239152_at | | 19.13 | 3.47e−05 | 6.06e−02 | ESTs | | Consensussequence | Hs.243023.0 | AW263526 |
| 35 | 221094_s_at | ELP3 | 18.93 | 9.28e−04 | 0.31 | likely ortholog of mouse | 8p21.1 | Exemplarsequence | Hs.267905.0 | NM_018091.1 |
| | | | | | | elongation protein 3 homolog (S. cerevisiae) | | | | |
| 36 | 221605_s_at | PIPOX | 18.78 | 8.74e−04 | 0.30 | pipecolic acid oxidase | 17q11.1 | Exemplarsequence | Hs.271167.1 | AF136970.1 |
| 37 | 238152_at | | 18.75 | 5.27e−05 | 7.16e−02 | ESTs, Weakly similar to collagen | | Consensussequence | Hs.195285.0 | AI683490 |
| | | | | | | [Caenorhabditis elegans] | | | | |
| | | | | | | [C. elegans] | | | | |
| 38 | 216974_at | | 18.68 | 4.35e−05 | 7.16e−02 | | | Consensussequence | Hs.123028.0 | S80491.1 |
| 39 | 210948_s_at | LEF1 | 18.60 | 8.72e−05 | 8.17e−02 | lymphoid enhancer-binding factor 1 | 4q23-q25 | Exemplarsequence | Hs.44865.0 | AF294627.1 |
| 40 | 202275_at | G6PD | 18.56 | 6.93e−05 | 7.16e−02 | glucose-6-phosphate | Xq28 | Exemplarsequence | Hs.80206.0 | NM_000402.1 |
| | | | | | | dehydrogenase | | | | |
| 41 | 231367_s_at | | 18.45 | 4.82e−04 | 0.22 | ESTs | | Consensussequence | Hs.28621.1 | AW300131 |
| 42 | 216268_s_at | JAG1 | 18.42 | 5.84e−05 | 7.16e−02 | jagged 1 (Alagille syndrome) | 20p12.1-p11.23 | Consensussequence | Hs.91143.3 | U77914.1 |
| 43 | 238418_at | YEA | 18.41 | 4.70e−05 | 7.16e−02 | YEA4 protein | 7q33 | Consensussequence | Hs.292566.0 | AI590926 |
| 44 | 206660_at | IGLL1 | 18.36 | 5.03e−05 | 7.16e−02 | immunoglobulin lambda-like | 22q11.23 | Exemplarsequence | Hs.288168.0 | NM_020070.1 |
| | | | | | | polypeptide 1 | | | | |
| 45 | 203585_at | ZNF185 | 18.35 | 5.40e−04 | 0.24 | zinc finger protein 185 (LIM | Xq28 | Exemplarsequence | Hs.16622.0 | NM_007150.1 |
| | | | | | | domain) | | | | |
| 46 | 206761_at | TACTILE | 18.33 | 6.33e−05 | 7.16e−02 | T cell activation, increased late | 3q13.13 | Exemplarsequence | Hs.142023.0 | NM_005816.1 |
| | | | | | | expression | | | | |
| 47 | 214819_at | KIAA0522 | 17.78 | 7.09e−05 | 7.16e−02 | KIAA0522 protein | Xp11.22 | Consensussequence | Hs.129892.0 | BF571239 |
| 48 | 235818_at | | 17.71 | 8.71e−05 | 8.17e−02 | ESTs, Weakly similar to T09402 | | Consensussequence | Hs.133355.0 | AI498747 |
| | | | | | | immunoglobulin-like protein | | | | |
| | | | | | | IGSF1 - human [H. sapiens] | | | | |
| 49 | 238709_at | | 17.69 | 6.73e−05 | 7.16e−02 | Homo sapiens cDNA FLJ38461 | | Consensussequence | Hs.170261.0 | AL041747 |
| | | | | | | fis, clone FEBRA2020977. | | | | |
| 50 | 202439_s_at | IDS | 17.59 | 6.22e−04 | 0.26 | iduronate 2-sulfatase (Hunter | Xq28 | Exemplarsequence | Hs.172458.0 | NM_000202.2 |
| | | | | | | syndrome) | | | | |

| | | Sequence | | | | |
|---|---|---|---|---|---|---|
| # | Sequence ID | Source | Unigene_Accession | Cluster_Type | LocusLink | Full_Length_Reference_Seq |
| 1 | Hs.143698.0_RC | GenBank | Hs.143698 | est | | |
| 2 | g6325464 | RefSeq | Hs.889 | fulllength | 1178 | NM_001828; Charot-Leyden crystal protein |
| 3 | g6006016 | RefSeq | Hs.79339 | fulllength | 3959 | NM_005567; galectin 3 binding protein |
| 4 | g13274521 | GenBank | Hs.119302 | fulllength | 114900 | NM_031909; C1q and tumor necrosis factor related |
| | | | | | | protein 4 |
| 5 | Hs.146144.0_RC | GenBank | Hs.146144 | est | | |
| 6 | g4557881 | RefSeq | Hs.84 | fulllength | 3561 | NM_000206; interleukin 2 receptor, gamma chain, |
| | | | | | | precursor |
| 7 | Hs.283931.0 | GenBank | | | | |
| 8 | Hs.137190.0_RC | GenBank | Hs.137190 | est | | |
| 9 | g13529121 | GenBank | Hs.82794 | fulllength | 1069 | NM_004344; caltractin |
| 10 | Hs.31319.0.A1 | GenBank | Hs.406173 | | | |
| 11 | Hs.156044.0 | GenBank | Hs.156044 | est | | |
| 12 | g13543541 | GenBank | Hs.99962 | fulllength | 5553 | NM_002728; proteoglycan 2 |
| 13 | Hs.87450.0.S1 | GenBank | Hs.87450 | fulllength | 1521 | NM_001335; cathepsin W preproprotein |
| 14 | g11038673 | RefSeq | Hs.89575 | fulllength | 974 | NM_000626; CD79B antigen isoform 1 precursor |
| | | | | | | NM_021602; CD79B antigen isoform 2 precursor |
| 15 | Hs.251946.0.S1 | GenBank | Hs.372714 | | 80336 | |
| 16 | Hs.123030.0 | GenBank | Hs.406565 | fulllength | 3514 | |
| 17 | Hs.121525.0.A1 | GenBank | Hs.121525 | est | | |
| 18 | Hs.46295.0 | GenBank | Hs.46295 | fulllength | 8288 | NM_000502; eosinophil peroxidase |
| 19 | Hs.148652.1_RC | GenBank | Hs.445480 | est | | |
| 20 | Hs.88162.0.A1 | GenBank | Hs.88162 | fulllength | 285313 | NM_178822; hypothetical protein FLJ25972 |
| 21 | Hs.102548.0_RC | GenBank | Hs.102548 | fulllength | 2909 | NM_004491; glucocorticoid receptor DNA binding |
| | | | | | | factor 1 isoform b NM_024342; glucocorticoid |
| | | | | | | receptor DNA binding factor 1 isoform a |
| 22 | Hs.83484.1.A1 | GenBank | Hs.83484 | fulllength | 6659 | NM_003107; SRY (sex determining region Y)-box 4 |
| 23 | g8923170 | RefSeq | Hs.114556 | fulllength | 54847 | NM_017699; hypothetical protein FLJ20174 |
| 24 | Hs.283390.0.S1 | GenBank | Hs.283390 | est | | |
| 25 | Hs.1098.0_RC | GenBank | Hs.1098 | fulllength | 54431 | NM_018981; ER-resident protein ERdj5 |
| 26 | Hs.248476.0_RC | GenBank | Hs.248476 | est | | |
| 27 | Hs.7911.1.S1 | GenBank | Hs.7911 | fulllength | 23351 | NM_020195; HCDI protein |
| 28 | Hs.190440.0_RC | GenBank | Hs.190440 | est | | |
| 29 | g12803748 | GenBank | Hs.25960 | fulllength | 4613 | NM_005378; v-myc myelocytomatosis viral related |
| | | | | | | oncogene, neuroblastoma derived |
| 30 | Hs.121706.0_RC | GenBank | Hs.4193 | fulllength | 26018 | NM_015541; leucine-rich repeats and |
| | | | | | | immunoglobulin-like domains 1 |
| 31 | Hs.103512.0.A1 | GenBank | Hs.103512 | fulllength | 124540 | NM_138962; musashi 2 isoform a NM_170721; |
| | | | | | | musashi 2 isoform b |
| 32 | g12804858 | GenBank | Hs.271966 | fulllength | 4241 | NM_005929; melanoma-associated antigen p97 |
| | | | | | | isoform 1, precursor NM_033316; melanoma- |
| | | | | | | associated antigen p97 isoform 2, precursor |
| 33 | g7427520 | RefSeq | Hs.73031 | fulllength | 5544 | NM_006249; proline-rich protein BstNI subfamily 3 |
| 34 | Hs.243023.0_RC | GenBank | Hs.445903 | est | | |
| 35 | g8922417 | RefSeq | Hs.267905 | fulllength | 55140 | NM_018091; elongation protein 3 homolog |
| 36 | g12239317 | GenBank | Hs.271167 | fulllength | 51268 | NM_016518; L-pipecolic acid oxidase |
| 37 | Hs.195285.0_RC | GenBank | Hs.380425 | est | | |
| 38 | Hs.123028.0 | GenBank | | | | |
| 39 | g11024343 | GenBank | Hs.44865 | fulllength | 51176 | NM_016269; lymphoid enhancer binding factor-1 |
| 40 | g4503844 | RefSeq | Hs.80206 | fulllength | 2539 | NM_000402; glucose-6-phosphate dehydrogenase |
| 41 | Hs.28621.1.A1 | GenBank | Hs.28621 | est | | |
| 42 | Hs.91143.3 | GenBank | Hs.91143 | fulllength | 182 | NM_000214; jagged 1 precursor |
| 43 | Hs.292566.0.A1 | GenBank | Hs.292566 | fulllength | 84912 | NM_032826; YEA4 protein |
| 44 | g13399297 | RefSeq | Hs.348935 | fulllength | 3543 | NM_020070; immunoglobulin lambda-like polypeptide |
| | | | | | | 1 isoform a precursor NM_152855; immunoglobulin |
| | | | | | | lambda-like polypeptide 1 isoform b precursor |
| 45 | g6005971 | RefSeq | Hs.16622 | fulllength | 7739 | NM_007150; zinc finger protein 185 (LIM domain) |
| 46 | g5032140 | RefSeq | Hs.142023 | fulllength | 10225 | NM_005816; T cell activation, increased late |
| | | | | | | expression |
| 47 | Hs.129892.0 | GenBank | Hs.129892 | | 23096 | |
| 48 | Hs.133355.0.A1 | GenBank | Hs.444431 | est | | |
| 49 | Hs.170261.0_RC | GenBank | Hs.170261 | | | |
| 50 | g5360215 | RefSeq | Hs.172458 | fulllength | 3423 | NM_000202; iduronate-2-sulfatase isoform a |
| | | | | | | precursor NM_006123; iduronate-2-sulfatase isoform |
| | | | | | | b precursor |

TABLE 32
_other

| # | affy id | HUGO name | Title | MapLocation | Sequence Type | Transcript ID | Sequence Derived From | Sequence ID |
|---|---|---|---|---|---|---|---|---|
| 1 | 232640_at | HT002 | HT002 protein; hypertension-related calcium- | 8q24-qter | Consensussequence | Hs.238928.2 | AK023070.1 | Hs.238928.2.S1 |
| | | | regulated gene | | | | | |
| 2 | 230361_at | | ESTs, Weakly similar to inner centromere | | Consensussequence | Hs.146109.0 | AW664013 | Hs.146109.0_RC |
| | | | protein [Mus musculus] [M. musculus] | | | | | |
| 3 | 222551_s_at | FLJ20989 | hypothetical protein FLJ20989 | | Consensussequence | Hs.169615.0 | AI197841 | Hs.169615.0 |
| 4 | 200632_s_at | NDRG1 | N-myc downstream regulated gene 1 | 8q24 | Exemplarsequence | Hs.75789.0 | NM_006096.1 | g5174656 |
| 5 | 226754_at | LOC90987 | hypothetical protein BC006258 | 8q24.3 | Consensussequence | Hs.285901.0 | W93231 | Hs.285901.0 |
| 6 | 219606_at | CGI-72 | CGI-72 protein | 8q24.3 | Exemplarsequence | Hs.318725.0 | NM_016018.1 | g7705782 |
| 7 | 217535_at | | | | Consensussequence | Hs.282721.0 | AV720514 | Hs.282721.0_RC |
| 8 | 209781_s_at | KHDRBS3 | KH domain containing, RNA binding, signal | 8q24.2 | Exemplarsequence | Hs.13565.0 | AF069681.1 | g3273831 |
| | | | transduction associated 3 | | | | | |
| 9 | 225488_at | MGC3067 | hypothetical protein MGC3067 | 8q24.13 | Consensussequence | Hs.323114.1 | AI967978 | Hs.323114.1 |
| 10 | 218858_at | FLJ12428 | hypothetical protein FL12428 | 8q24.12 | Exemplarsequence | Hs.87729.0 | NM_022783.1 | g12232472 |
| 11 | 230555_s_at | PFDN2 | prefoldin 2 | 1q23.1 | Consensussequence | Hs.298229.1 | AA521496 | Hs.298229.1.A1 |
| 12 | 206573_at | KCNQ3 | potassium voltage-gated channel, KQT-like | 8q24 | Exemplarsequence | Hs.40866.0 | NM_004519.1 | g4758629 |
| | | | subfamily, member 3 | | | | | |
| 13 | 235205_at | | Homo sapiens, clone IMAGE: 5726657, | | Consensussequence | Hs.127286.0 | BF109660 | Hs.127286.0.A1 |
| | | | mRNA | | | | | |
| 14 | 212865_s_at | COL14A1 | collagen, type XIV, alpha 1 (undulin) | 8q23 | Consensussequence | Hs.36131.0 | BF449063 | Hs.36131.0.S1 |
| 15 | 225439_at | CML66 | chronic myelogenous leukemia tumor | 8q23 | Consensussequence | Hs.195870.0 | BC000967.2 | Hs.195870.0 |
| | | | antigen 66 | | | | | |
| 16 | 216865_at | COL14A1 | collagen, type XIV, alpha 1 (undulin) | 8q23 | Consensussequence | Hs.36131.2 | M64108.1 | Hs.36131.2.S1 |
| 17 | 225438_at | CML66 | chronic myelogenous leukemia tumor | 8q23 | Consensussequence | Hs.195870.0 | BC000967.2 | Hs.195870.0 |
| | | | antigen 66 | | | | | |
| 18 | 201754_at | COX6C | cytochrome c oxidase subunit VIc | 8q22-q23 | Exemplarsequence | Hs.74649.0 | NM_004374.1 | g4758039 |
| 19 | 205608_s_at | ANGPT1 | angiopoietin 1 | 8q22.3-q23 | Exemplarsequence | Hs.2463.0 | U83508.1 | g1907326 |
| 20 | 205609_at | ANGPT1 | angiopoietin 1 | 8q22.3-q23 | Exemplarsequence | Hs.2463.0 | NM_001146.1 | g4502086 |
| 21 | 220843_s_at | DKFZP564O0463 | DKFZP564O0463 protein | 8q22.3 | Exemplarsequence | Hs.273344.0 | NM_014156.1 | g7661767 |
| 22 | 225676_s_at | DKFZP564O0463 | DKFZP564O0463 protein | 8q22.3 | Consensussequence | Hs.273344.1 | AK001693.1 | Hs.273344.1 |
| 23 | 202872_at | ATP6V1C1 | ATPase, H+ transporting, lysosomal 42 kDa, | 8q22.3 | Consensussequence | Hs.86905.0 | NM_001695.1 | Hs.86905.0 |
| | | | V1 subunit C, isoform 1 | | | | | |
| 24 | 202634_at | POLR2K | polymerase (RNA) II (DNA directed) | 8q22.2 | Consensussequence | Hs.150675.0 | AL558030 | Hs.150675.0 |
| | | | polypeptide K, 7.0 kDa | | | | | |
| 25 | 220254_at | ST7 | suppression of tumorigenicity | 8q22.2-q23.1 | Exemplarsequence | Hs.301974.0 | NM_013437.1 | g7305524 |
| 26 | 220128_s_at | FLJ13955 | hypothetical protein FLJ13955 | 8q22.1 | Exemplarsequence | Hs.127331.0 | NM_024759.1 | g13376096 |
| 27 | 211078_s_at | STK3 | serine/threonine kinase 3 (STE20 homolog, | 8q22.1 | Exemplarsequence | g405722 | Z25422.1 | g405722 |
| | | | yeast) | | | | | |
| 28 | 235509_at | MGC40214 | hypothetical protein MGC40214 | 8q22.1 | Consensussequence | Hs.98471.0 | AV662196 | Hs.98471.0_RC |
| 29 | 239860_at | | ESTs | | Consensussequence | Hs.16292.0 | AI311917 | Hs.16292.0.A1 |
| 30 | 205849_s_at | UQCRB | ubiquinol-cytochrome c reductase binding | 8q22 | Exemplarsequence | Hs.131255.0 | NM_006294.1 | g5454151 |
| | | | protein | | | | | |
| 31 | 209066_x_at | UQCRB | ubiquinol-cytochrome c reductase binding | 8q22 | Exemplarsequence | Hs.131255.1 | M26700.1 | g190803 |
| | | | protein | | | | | |
| 32 | 231268_at | | Homo sapiens, clone IMAGE: 5222754, | | Consensussequence | Hs.144027.0 | AI539459 | Hs.144027.0.A1 |
| | | | mRNA, partial cds | | | | | |
| 33 | 213906_at | MYBL1 | v-myb myeloblastosis viral oncogene | 8q22 | Consensussequence | Hs.300592.0 | AW592266 | Hs.300592.0.S1 |
| | | | homolog (avian)-like 1 | | | | | |
| 34 | 242338_at | DKFZp762C1112 | hypothetical protein DKFZp762C1112 | 8q21.3 | Consensussequence | Hs.330692.0 | BG535396 | Hs.330692.0.S1 |
| 35 | 202447_at | DECR1 | 2,4-dienoyl CoA reductase 1, mitochondrial | 8q21.3 | Exemplarsequence | Hs.81548.0 | NM_001359.1 | g4503300 |
| 36 | 218549_s_at | CGI-90 | CGI-90 protein | 8q21.13 | Exemplarsequence | Hs.44222.0 | NM_016033.1 | g7705802 |
| 37 | 219819_s_at | MRPS28 | mitochondrial ribosomal protein S28 | 8q21.1-q21.2 | Exemplarsequence | Hs.55097.0 | NM_014018.1 | g7661729 |
| 38 | 202345_s_at | FABP5 | fatty acid binding protein 5 (psoriasis- | 8q21.11 | Exemplarsequence | Hs.153179.0 | NM_001444.1 | g4557580 |
| | | | associated) | | | | | |
| 39 | 205308_at | CGI-62 | CGI-62 protein | 8q21.11 | Exemplarsequence | Hs.118821.0 | NM_016010.1 | g7705774 |
| 40 | 226864_at | PKIA | protein kinase (cAMP-dependent, catalytic) | 8q21.11 | Consensussequence | Hs.75209.1 | BF245954 | Hs.75209.1.S1 |
| | | | inhibitor alpha | | | | | |
| 41 | 201691_s_at | TPD52 | tumor protein D52 | 8q21 | Exemplarsequence | Hs.2384.0 | NM_005079.1 | g4827037 |
| 42 | 222863_at | RINZF | zinc finger protein RINZF | 8q13-q21.1 | Consensussequence | Hs.237146.0 | BG483802 | Hs.237146.0.S2 |
| 43 | 229417_at | STAU2 | staufen, RNA binding protein, homolog 2 | 8q13-q21.1 | Consensussequence | Hs.96870.2 | W46994 | Hs.96870.2_RC |
| | | | (Drosophila) | | | | | |
| 44 | 235210_s_at | FLJ40021 | hypothetical protein FLJ40021 | 8q13.2 | Consensussequence | Hs.122544.0 | AW662373 | Hs.122544.0_RC |
| 45 | 219448_at | FLJ20533 | hypothetical protein FLJ20533 | 8q13.3 | Exemplarsequence | Hs.106650.0 | BC002748.1 | g12803814 |
| 46 | 226021_at | | ESTs, Weakly similar to hypothetical protein | | Consensussequence | Hs.106283.0 | AW150720 | Hs.106283.0.A1 |
| | | | FLJ20489 [Homo sapiens] [H. sapiens] | | | | | |
| 47 | 239493_at | RPL7 | ribosomal protein L7 | 8q13.2 | Consensussequence | Hs.196398.0 | AI984074 | Hs.196398.0_RC |
| 48 | 220984_s_at | OATPRP4 | organic anion transporter polypeptide-related | 8q13.1 | Exemplarsequence | g13569931 | NM_030958.1 | g13569931 |
| | | | protein 4 | | | | | |
| 49 | 210614_at | TTPA | tocopherol (alpha) transfer protein (ataxia | 8q13.1-q13.3 | Exemplarsequence | Hs.69049.0 | U21938.1 | g726181 |
| | | | (Friedreich-like) with vitamin E deficiency) | | | | | |
| 50 | 201652_at | COPS5 | COP9 constitutive photomorphogenic | 8q12.3 | Exemplarsequence | Hs.198767.0 | NM_006837.1 | g5803045 |
| | | | homolog subunit 5 (Arabidopsis) | | | | | |
| 51 | 203208_s_at | CHPPR | likely ortholog of chicken chondrocyte protein | 8q12.1 | Exemplarsequence | Hs.170198.0 | NM_014637.1 | g7661853 |
| | | | with a poly-proline region | | | | | |
| 52 | 203207_s_at | CHPPR | likely ortholog of chicken chondrocyte protein | 8q12.1 | Consensussequence | Hs.170198.0 | BF214329 | Hs.170198.0.S1 |
| | | | with a poly-proline region | | | | | |
| 53 | 226123_at | LOC286180 | hypothetical protein LOC286180 | 8q12.1 | Consensussequence | Hs.60238.0 | AI870918 | Hs.60238.0_RC |
| 54 | 204529_s_at | TOX | thymus high mobility group box protein TOX | 8q11.23 | Consensussequence | Hs.184297.0 | AI961231 | Hs.184297.0.S1 |
| 55 | 200949_x_at | RPS20 | ribosomal protein S20 | 8q12 | Exemplarsequence | Hs.8102.0 | NM_001023.1 | g4506696 |
| 56 | 234919_s_at | SNTG1 | syntrophin, gamma 1 | 8q11-q12 | Consensussequence | Hs.167481.1 | AJ003030.1 | Hs.167481.1 |
| 57 | 228734_at | | ESTs | | Consensussequence | Hs.30662.0 | BF447286 | Hs.30662.0_RC |
| 58 | 222036_s_at | MCM4 | MCM4 minichromosome maintenance | 8q12-q13 | Consensussequence | Hs.154443.1 | AI859865 | Hs.154443.1.S1 |
| | | | deficient 4 (S. cerevisiae) | | | | | |
| 59 | 239877_at | MTMR9 | myotubularin related protein 9 | 8p23-p22 | Consensussequence | Hs.128277.0 | AI499833 | Hs.128277.0_RC |
| 60 | 226614_s_at | C8orf13 | chromosome 8 open reading frame 13 | 8p23-p22 | Consensussequence | Hs.35453.0 | BE856336 | Hs.35453.0.A1 |
| 61 | 229958_at | | Homo sapiens cDNA FLJ39417 fis, clone | | Consensussequence | Hs.14880.0 | W93695 | Hs.14880.0_RC |
| | | | PLACE6016942. | | | | | |
| 62 | 210397_at | DEFB1 | defensin, beta 1 | 8p23.2-p23.1 | Exemplarsequence | Hs.32949.0 | U73945.1 | g1755147 |
| 63 | 243692_at | GATA4 | GATA binding protein 4 | 8p23.1-p22 | Consensussequence | Hs.194114.0 | AW181962 | Hs.194114.0_RC |
| 64 | 237261_at | | ESTs, Highly similar to hypothetical protein | | Consensussequence | Hs.71832.0 | BE501356 | Hs.71832.0.A1 |
| | | | FLJ12847 [Homo sapiens] [H. sapiens] | | | | | |
| 65 | 223907_s_at | PINX1 | PIN2-interacting protein 1 | 8p23 | Exemplarsequence | Hs.99829.0 | AF205718.1 | g10504237 |
| 66 | 222714_s_at | CGI-83 | CGI-83 protein | 8p22-q22.3 | Exemplarsequence | Hs.118554.0 | BC000878.1 | g12654126 |
| 67 | 209294_x_at | TNFRSF10B | tumor necrosis factor receptor superfamily, | 8p22-p21 | Exemplarsequence | Hs.51233.0 | BC001281.1 | g12654874 |
| | | | member 10b | | | | | |
| 68 | 208423_s_at | MSR1 | macrophage scavenger receptor 1 | 8p22 | Exemplarsequence | Hs.49.0 | NM_002445.1 | g4505258 |
| 69 | 209227_at | N33 | Putative prostate cancer tumor suppressor | 8p22 | Consensussequence | Hs.71119.0 | AU158251 | Hs.71119.0.A2 |
| 70 | 238458_at | LOC286097 | hypothetical protein LOC286097 | 8p22 | Consensussequence | Hs.131044.0 | AI868167 | Hs.131044.0.S1 |
| 71 | 235801_at | N33 | Putative prostate cancer tumor suppressor | 8p22 | Consensussequence | Hs.283526.0 | AI760262 | Hs.283526.0_RC |
| 72 | 218613_at | DKFZp761K1423 | hypothetical protein DKFZp761K1423 | 8p21.3 | Exemplarsequence | Hs.236438.0 | NM_018422.1 | g8922171 |
| 73 | 230977_at | LOC286056 | hypothetical protein LOC286056 | 8p21.2 | Consensussequence | Hs.131055.0 | AI016313 | Hs.131055.0.A1 |
| 74 | 219665_at | FLJ22494 | hypothetical protein FLJ22494 | 8p21.2 | Exemplarsequence | Hs.170132.0 | NM_024815.1 | g13376205 |
| 75 | 207287_at | FLJ14107 | hypothetical protein FLJ14107 | 8p21.2 | Exemplarsequence | Hs.287624.0 | NM_025026.1 | g13376547 |
| 76 | 239303_at | | ESTs | | Consensussequence | Hs.97814.0 | AA933717 | Hs.97814.0.A1 |
| 77 | 209697_at | PPP3CC | protein phosphatase 3 (formerly 2B), | 8p21.2 | Consensussequence | Hs.75206.1 | BC004864.1 | Hs.75206.1.A1 |
| | | | catalytic subunit, gamma isoform (calcineurin | | | | | |
| | | | A gamma) | | | | | |
| 78 | 219921_s_at | FLJ21034 | hypothetical protein FLJ21034 | 8p21.1 | Exemplarsequence | Hs.282466.0 | NM_024940.1 | g13376416 |
| 79 | 224316_at | FLJ20038 | hypothetical protein FLJ20038 | 8p21.1 | Exemplarsequence | Hs.72071.1 | AF130091.1 | g11493486 |
| 80 | 222525_s_at | FLJ10853 | hypothetical protein FLJ10853 | 8p21.1 | Consensussequence | Hs.72085.0 | AU160632 | Hs.72085.0 |
| 81 | 203941_at | FLJ10871 | hypothetical protein FLJ10871 | 8p12 | Exemplarsequence | Hs.15562.0 | NM_018250.1 | g8922725 |
| 82 | 241371_at | | ESTs | | Consensussequence | Hs.57851.0 | AW451259 | Hs.57851.0.A1 |
| 83 | 231775_at | | ESTs, Weakly similar to neuronal thread | | Consensussequence | Hs.249190.0 | W65310 | Hs.249190.0.S2 |
| | | | protein [Homo sapiens] [H. sapiens] | | | | | |
| 84 | 215983_s_at | D8S2298E | reproduction 8 | 8p12-p11.2 | Consensussequence | Hs.153678.1 | D83768.1 | Hs.153678.1.S1 |
| 85 | 210029_at | INDO | indoleamine-pyrrole 2,3 dioxygenase | 8p12-p11 | Exemplarsequence | Hs.840.0 | M34455.1 | g185790 |
| 86 | 231961_at | | Homo sapiens cDNA FLJ14130 fis, clone | | Consensussequence | Hs.112572.0 | AU148114 | Hs.112572.0.S1 |
| | | | MAMMA1002618. | | | | | |
| 87 | 204014_at | DUSP4 | dual specificity phosphatase 4 | 8p12-p11 | Exemplarsequence | Hs.2359.0 | NM_001394.2 | g12707552 |
| 88 | 207737_at | 1D12A | pre-T/NK cell associated protein | 8p12 | Exemplarsequence | Hs.278.0 | NM_021981.1 | g11415055 |
| 89 | 242904_x_at | MGC8721 | hypothetical protein MGC8721 | 8p12 | Consensussequence | Hs.209078.0 | AI351653 | Hs.209078.0.A1 |
| 90 | 210973_s_at | FGFR1 | fibroblast growth factor receptor 1 (fms- | 8p11.2-p11.1 | Exemplarsequence | Hs.748.7 | M63889.1 | g183882 |
| | | | related tyrosine kinase 2, Pfeiffer syndrome) | | | | | |
| 91 | 232040_at | | Homo sapiens cDNA: FLJ22090 fis, clone | | Consensussequence | Hs.170296.0 | AK025743.1 | Hs.170296.0 |
| | | | HEP16084. | | | | | |
| 92 | 222760_at | FLJ14299 | hypothetical protein FLJ14299 | 8p11.22 | Consensussequence | Hs.288042.0 | BG290193 | Hs.288042.0_RC |
| 93 | 203534_at | LSM1 | LSM1 homolog, U6 small nuclear RNA associated (S. cerevisiae) | 8p11.2 | Exemplarsequence | Hs.111783.0 | NM_014462.1 | g7657312 |
| 94 | 208846_s_at | VDAC3 | voltage-dependent anion channel 3 | 8p11.2 | Exemplarsequence | Hs.7381.0 | U90943.1 | g2735306 |
| 95 | 224127_at | C8orf4 | chromosome 8 open reading frame 4 | 8p11.2 | Exemplarsequence | Hs.31446.0 | AF116660.1 | g7959818_RC |

| | Sequence | | | | |
|---|---|---|---|---|---|
| # | Source | Unigene_Accession | Cluster_Type | LocusLink | Full_Length_Reference_Seq |
| 1 | GenBank | Hs.238928 | fulllength | 28991 | NM_014066; hypertension-related calcium-regulated gene |
| 2 | GenBank | Hs.443139 | est | | |
| 3 | GenBank | Hs.169615 | fulllength | 65265 | NM_023080; hypothetical protein FLJ20989 |
| 4 | RefSeq | Hs.75789 | fulllength | 10397 | NM_006096; N-myc downstream regulated gene 1 |
| 5 | GenBank | Hs.285901 | | 90987 | |
| 6 | RefSeq | Hs.318725 | fulllength | 51105 | NM_016018; CGI-72 protein |
| 7 | GenBank | | | | |
| 8 | GenBank | Hs.13565 | fulllength | 10656 | NM_006558; KH domain containing, RNA binding, signal transduction associated 3 |
| 9 | GenBank | Hs.323114 | fulllength | 79139 | NM_018630; NM_024295; hypothetical protein MGC3067 |
| 10 | RefSeq | Hs.87729 | fulllength | 64798 | NM_022783; hypothetical protein FLJ12428 |
| 11 | GenBank | Hs.298229 | fulllength | 5202 | NM_012394; prefoldin 2 NM_080651; TRAP/Mediator complex component TRAP25 |
| 12 | RefSeq | Hs.40866 | fulllength | 3786 | NM_004519; potassium voltage-gated channel KQT-like protein 3 |
| 13 | GenBank | Hs.127286 | | | |
| 14 | GenBank | Hs.403836 | fulllength | 7373 | |
| 15 | GenBank | Hs.195870 | fulllength | 84955 | NM_032869; chronic myelogenous leukemia tumor antigen 66 |
| 16 | GenBank | Hs.403836 | fulllength | 7373 | |
| 17 | GenBank | Hs.195870 | fulllength | 84955 | NM_032869; chronic myelogenous leukemia tumor antigen 66 |
| 18 | RefSeq | Hs.351875 | fulllength | 1345 | NM_004374; cytochrome c oxidase subunit VIc proprotein |
| 19 | GenBank | Hs.2463 | fulllength | 284 | NM_001146; angiopoietin 1 isoform a NM_139290; angiopoietin 1 isoform b |
| 20 | RefSeq | Hs.2463 | fulllength | 284 | NM_001146; angiopoietin 1 isoform a NM_139290; angiopoietin 1 isoform b |
| 21 | RefSeq | Hs.273344 | fulllength | 25879 | NM_014156; NM_015420; DKFZP564O0463 protein |
| 22 | GenBank | Hs.273344 | fulllength | 25879 | NM_014156; NM_015420; DKFZP564O0463 protein |
| 23 | GenBank | Hs.86905 | fulllength | 528 | NM_001695; ATPase, H+ transporting, lysosomal 42 kD, V1 subunit C, isoform 1 |
| 24 | GenBank | Hs.351475 | fulllength | 5440 | NM_005034; DNA directed RNA polymerase II polypeptide K |
| 25 | RefSeq | Hs.301974 | fulllength | 29967 | NM_013437; suppression of tumorigenicity NM_024937; |
| 26 | RefSeq | Hs.127331 | fulllength | 79815 | NM_024759; hypothetical protein FLJ13955 |
| 27 | GenBank | Hs.166684 | fulllength | 6788 | NM_006281; serine/threonine kinase 3 (STE20 homolog, yeast) |
| 28 | GenBank | Hs.98471 | est | 137682 | NM_152416; hypothetical protein MGC40214 |
| 29 | GenBank | Hs.16292 | est | | |
| 30 | RefSeq | Hs.131255 | fulllength | 7381 | NM_006294; ubiquinol-cytochrome c reductase binding protein |
| 31 | GenBank | Hs.131255 | fulllength | 7381 | NM_006294; ubiquinol-cytochrome c reductase binding protein |
| 32 | GenBank | Hs.144027 | | | |
| 33 | GenBank | Hs.300592 | | 4603 | |
| 34 | GenBank | Hs.88594 | | 169200 | |
| 35 | RefSeq | Hs.81548 | fulllength | 1666 | NM_001359; 2,4-dienoyl CoA reductase 1 precursor |
| 36 | RefSeq | Hs.44222 | fulllength | 51115 | NM_016033; CGI-90 protein |
| 37 | RefSeq | Hs.55097 | fulllength | 28957 | NM_014018; mitochondrial ribosomal protein S28 |
| 38 | RefSeq | Hs.408061 | fulllength | 2171 | NM_001444; fatty acid binding protein 5(psoriasis-associated) |
| 39 | RefSeq | Hs.118821 | fulllength | 51101 | NM_016010; CGI-62 protein |
| 40 | GenBank | Hs.433700 | fulllength | 5569 | NM_006823; protein kinase, cAMP-dependent, catalytic, inhibitor alpha |
| 41 | RefSeq | Hs.2384 | fulllength | 7163 | NM_005079; tumor protein D52 |
| 42 | GenBank | Hs.237146 | fulllength | 65986 | NM_023929; zinc finger protein RINZF |
| 43 | GenBank | Hs.96870 | fulllength | 27067 | NM_014393; staufen homolog 2 |
| 44 | GenBank | Hs.41185 | fulllength | 157869 | NM_153225; hypothetical protein FLJ40021 |
| 45 | GenBank | Hs.106650 | fulllength | 54968 | NM_017866; hypothetical protein FLJ20533 |
| 46 | GenBank | Hs.356086 | est | | |
| 47 | GenBank | Hs.153 | fulllength | 6129 | NM_000971; ribosomal protein L7 |
| 48 | RefSeq | Hs.199750 | fulllength | 81796 | NM_030958; organic anion transporter polypeptide-retated protein 4 |
| 49 | GenBank | Hs.69049 | fulllength | 7274 | NM_000370; tocopherol (alpha) transfer protein (ataxia (Friedreich-like) with vitamin E deficiency) |
| 50 | RefSeq | Hs.380969 | fulllength | 10987 | NM_006837; COP9 constitutive photomorphogenic homolog subunit 5 |
| 51 | RefSeq | Hs.170198 | fulllength | 9650 | NM_014637; KIAA0009 gene product |
| 52 | GenBank | Hs.170198 | fulllength | 9650 | NM_014637; KIAA0009 gene product |
| 53 | GenBank | Hs.397426 | | 286180 | |
| 54 | GenBank | Hs.184297 | fulllength | 9760 | NM_014729; thymus high mobility group box protein TOX |
| 55 | RefSeq | Hs.8102 | fulllength | 6224 | NM_001023; ribosomal protein S20 |
| 56 | GenBank | Hs.167481 | fulllength | 54212 | NM_018967; syntrophin, gamma 1 |
| 57 | GenBank | Hs.371278 | est | | |
| 58 | GenBank | Hs.154443 | fulllength | 4173 | |
| 59 | GenBank | Hs.48802 | fulllength | 66036 | NM_015458; myotubularin related protein 9 |
| 60 | GenBank | Hs.35453 | | 83648 | |
| 61 | GenBank | Hs.14880 | | | |
| 62 | GenBank | Hs.32949 | fulllength | 1672 | NM_005218; defensin, beta 1, preproprotein |
| 63 | GenBank | Hs.243987 | fulllength | 2626 | NM_002052; GATA binding protein 4 |
| 64 | GenBank | Hs.71832 | est | | |
| 65 | GenBank | Hs.400259 | fulllength | 54984 | NM_017884; PIN2-interacting protein 1 |
| 66 | GenBank | Hs.118554 | fulllength | 51110 | NM_016027; lactamase, beta 2 |
| 67 | GenBank | Hs.51233 | fulllength | 8795 | NM_003842; tumor necrosis factor receptor superfamily, member 10b isoform 1 precursor NM_147187; |
| | | | | | tumor necrosis factor receptor superfamily, member 10b isoform 2 precursor |
| 68 | RefSeq | Hs.49 | fulllength | 4481 | NM_002445; macrophage scavenger receptor 1 isoform type 2 NM_138715; macrophage scavenger receptor |
| | | | | | 1 isoform type 1 NM_138716; macrophage scavenger receptor 1 isoform type 3 |
| 69 | GenBank | Hs.71119 | fulllength | 7991 | NM_006765; Putative prostate cancer tumor suppressor isoform a NM_178234; Putative prostate cancer |
| | | | | | tumor suppressor isoform b |
| 70 | GenBank | Hs.131044 | fulllength | 286097 | |
| 71 | GenBank | Hs.71119 | fulllength | 7991 | NM_006765; Putative prostate cancer tumor suppressor isoform a NM_178234; Putative prostate cancer |
| | | | | | tumor suppressor isoform b |
| 72 | RefSeq | Hs.236438 | fulllength | 55358 | NM_018422; hypothetical protein DKFZp761K1423 |
| 73 | GenBank | Hs.131055 | | 286056 | |
| 74 | RefSeq | Hs.170132 | fulllength | 79873 | NM_024815; hypothetical protein FLJ22494 |
| 75 | RefSeq | Hs.287624 | fulllength | 80094 | NM_025026; hypothetical protein FLJ14107 |
| 76 | GenBank | Hs.446548 | est | | |
| 77 | GenBank | Hs.75206 | fulllength | 5533 | NM_005605; protein phosphatase 3 (formerly 2B), catalytic subunit, gamma isoform (calcineurin A gamma) |
| 78 | RefSeq | Hs.282466 | fulllength | 80005 | NM_024940; hypothetical protein FLJ21034 |
| 79 | GenBank | Hs.72071 | fulllength | 54793 | NM_017634; hypothetical protein FLJ20038 |
| 80 | GenBank | Hs.72085 | fulllength | 55246 | NM_018246; hypothetical protein FLJ10853 |
| 81 | RefSeq | Hs.15562 | fulllength | 55756 | NM_018250; hypothetical protein FLJ10871 |
| 82 | GenBank | Hs.444578 | est | | |
| 83 | GenBank | Hs.401745 | est | | |
| 84 | GenBank | Hs.153678 | fulllength | 7993 | NM_005671; reproduction 8 |
| 85 | GenBank | Hs.840 | fulllength | 3620 | NM_002164; indoleamine-pyrrole 2,3 dioxygenase |
| 86 | GenBank | Hs.112572 | | | |
| 87 | RefSeq | Hs.2359 | fulllength | 1846 | NM_001394; dual specificity phosphatase 4 isoform 1 NM_057158; dual specificity phosphatase 4 isoform 2 |
| 88 | RefSeq | Hs.278 | fulllength | 9594 | NM_021981; pre-T/NK cell associated protein 1D12A |
| 89 | GenBank | Hs.279921 | fulllength | 51669 | NM_016127; hypothetical protein MGC8721 |
| 90 | GenBank | Hs.748 | fulllength | 2260 | NM_000604; fibroblast growth factor receptor 1 isoform 1 precursor NM_015850; fibroblast growth factor |
| | | | | | receptor 1 isoform 2 precursor NM_023105; fibroblast growth factor receptor 1 isoform 3 precursor |
| | | | | | NM_023106; fibroblast growth factor receptor 1 isoform 4 precursor NM_023107; fibroblast growth factor |
| | | | | | receptor 1 isoform 5 precursor NM_023108; fibroblast growth factor receptor 1 isoform 6 precursor |
| | | | | | NM_023109; fibroblast growth factor receptor 1 isoform 7 precursor NM_023110; fibroblast growth factor |
| | | | | | receptor 1 isoform 8 precursor NM_023111; fibroblast growth factor receptor 1 isoform 9 precursor |
| 91 | GenBank | Hs.170296 | | | |
| 92 | GenBank | Hs.288042 | fulllength | 80139 | NM_025069; hypothetical protein FLJ14299 |
| 93 | RefSeq | Hs.425311 | fulllength | 27257 | NM_014462; Lsm1 protein |
| 94 | GenBank | Hs.7381 | fulllength | 7419 | NM_005662; voltage-dependent anion channel 3 |
| 95 | GenBank | Hs.283683 | fulllength | 56892 | NM_020130; chromosome 8 open reading frame 4 |

TABLE 33
_complex

| # | affy id | HUGO name | F | p | q | Title | MapLocation | Sequence Type | Transcript ID |
|---|---|---|---|---|---|---|---|---|---|
| 1 | 231585_at | CHAC | 36.79 | 2.29e−07 | 5.65e−03 | chorea acanthocytosis | 9q21 | Consensussequence | Hs.65919.1 |
| 2 | 231004_s_at | TGM2 | 31.58 | 2.57e−07 | 5.65e−03 | transglutaminase 2 (C polypeptide, | 20q12 | Consensussequence | Hs.109804.1 |
| | | | | | | protein-glutamine-gamma-glutamyltransferase) | | | |
| 3 | 200090_at-HG-U133B | FNTA | 30.63 | 9.14e−05 | 0.10 | famesyltransferase, CAAX box, alpha | 8p22-q11 | Consensussequence | Hs.138381.1 |
| 4 | 220055_at | ZNF287 | 30.49 | 4.89e−07 | 7.16e−03 | zinc finger protein 287 | 17p11.2 | Exemplarsequence | Hs.99724.0 |
| 5 | 215052_at | | 28.32 | 9.75e−07 | 1.07e−02 | ESTs | | Consensussequence | Hs.92025.0 |
| 6 | 200923_at | LGALS3BP | 27.20 | 1.35e−06 | 1.19e−02 | lectin, galactoside-binding, soluble, 3 | 17q25 | Exemplarsequence | Hs.79339.0 |
| | | | | | | binding protein | | | |
| 7 | 238715_at | | 27.15 | 3.23e−06 | 1.77e−02 | Homo sapiens; clone IMAGE: 4798235, | | Consensussequence | Hs.117853.0 |
| | | | | | | mRNA | | | |
| 8 | 244817_at | ING1 | 26.62 | 2.61e−06 | 1.77e−02 | inhibitor of growth family, member 1 | 13q34 | Consensussequence | Hs.28714.0 |
| 9 | 215209_at | | 26.16 | 3.16e−06 | 1.77e−02 | Homo sapiens, clone IMAGE: 5265853, | | Consensussequence | Hs.301645.0 |
| | | | | | | mRNA | | | |
| 10 | 244364_at | MYO3A | 25.51 | 1.08e−05 | 3.95e−02 | myosin IIIA | 10p11.1 | Consensussequence | Hs.98995.0 |
| 11 | 240068_at | | 23.97 | 4.84e−06 | 2.36e−02 | Homo sapiens cDNA FLJ37173 fis, | | Consensussequence | Hs.106234.0 |
| | | | | | | clone BRACE2028392. | | | |
| 12 | 241618_at | | 23.86 | 8.26e−06 | 3.30e−02 | ESTs | | Consensussequence | Hs.133292.0 |
| 13 | 226462_at | STXBP6 | 23.34 | 6.31e−06 | 2.77e−02 | syntaxin binding protein 6 (amisyn) | 14q11.2 | Consensussequence | Hs.99291.1 |
| 14 | 207552_at | ATP5G2 | 22.48 | 2.62e−05 | 5.77e−02 | ATP synthase, H+ transporting, | 12q13.13 | Exemplarsequence | Hs.89399.0 |
| | | | | | | mitochondrial F0 complex, subunit c | | | |
| | | | | | | (subunit 9), isoform 2 | | | |
| 15 | 240761_at | | 22.32 | 2.50e−05 | 5.77e−02 | ESTs | | Consensussequence | Hs.147260.0 |
| 16 | 229023_at | MGC5391 | 22.18 | 1.40e−05 | 4.30e−02 | hypothetical protein MGC5391 | 2q14.3 | Consensussequence | Hs.55256.0 |
| 17 | 211265_at | PTGER3 | 21.80 | 1.20e−05 | 4.05e−02 | prostaglandin E receptor 3 (subtype | 1p31.2 | Exemplarsequence | Hs.170917.3 |
| | | | | | | EP3) | | | |
| 18 | 214740_at | POLR2J2 | 21.77 | 2.94e−05 | 5.86e−02 | DNA directed RNA polymerase II | 7q11.22 | Consensussequence | Hs.80475.2 |
| | | | | | | polypeptide J-related gene | | | |
| 19 | 242356_at | | 21.60 | 1.85e−05 | 4.92e−02 | Homo sapiens cDNA FLJ38157 fis, | | Consensussequence | Hs.120654.0 |
| | | | | | | clone DFNES2000426. | | | |
| 20 | 201443_s_at | ATP6IP2 | 21.31 | 1.40e−04 | 0.11 | ATPase, H+ transporting, lysosomal | Xq21 | Esemplarsequence | Hs.183434.0 |
| | | | | | | interacting protein 2 | | | |
| 21 | 204289_at | ALDH6A1 | 21.28 | 1.47e−05 | 4.30e−02 | aldehyde dehydrogenase 6 family, | 14q24.3 | Consensussequence | Hs.293970.0 |
| | | | | | | member A1 | | | |
| 22 | 217589_at | RAB40A | 20.70 | 5.89e−05 | 8.62e−02 | RAB40A, member RAS oncogene | Xq22.1 | Consensussequence | Hs.27453.0 |
| | | | | | | family | | | |
| 23 | 204013_s_at | LCMT2 | 20.64 | 1.90e−05 | 4.92e−02 | leucine carboxyl methyltransferase 2 | 15q14 | Exemplarsequence | Hs.200596.0 |
| 24 | 201004_at | SSR4 | 20.48 | 6.28e−05 | 8.62e−02 | signal sequence receptor, delta | Xq28 | Exemplarsequence | Hs.102135.0 |
| | | | | | | (translocon-associated protein delta) | | | |
| 25 | 207067_s_at | HDC | 20.39 | 2.56e−05 | 5.77e−02 | histidine decarboxylase | 15q21-q22 | Exemplarsequence | Hs.1481.0 |
| 26 | 240930_at | | 20.35 | 5.26e−05 | 8.56e−02 | ESTs | | Consensussequence | Hs.117900.0 |
| 27 | 206107_at | RGS11 | 20.11 | 2.76e−05 | 5.78e−02 | regulator of G-protein signalling 11 | 16p13.3 | Exemplarsequence | Hs.65756.0 |
| 28 | 206282_at | NEUROD1 | 19.39 | 3.44e−05 | 6.57e−02 | neurogenic differentiation 1 | 2q32 | Exemplarsequence | Hs.72981.0 |
| 29 | 239001_at | | 19.30 | 6.25e−05 | 8.62e−02 | ESTs | | Consensussequence | Hs.132678.0 |
| 30 | 215576_at | | 18.98 | 7.85e−05 | 9.37e−02 | Homo sapiens cDNA FLJ12004 fis, clone HEMBB1001564, | | Consensussequence | Hs.287476.0 |
| | | | | | | moderately similar to VACUOLAR ATP SYNTHASE SUBUNIT H | | | |
| | | | | | | (EC 3.6.1.34). | | | |
| 31 | 237480_at | | 18.77 | 4.51e−05 | 8.26e−02 | ESTs | | Consensussequence | Hs.213049.0 |
| 32 | 235586_at | | 18.55 | 6.25e−05 | 8.62e−02 | ESTs | | Consensussequence | Hs.23892.0 |
| 33 | 215108_x_at | TNRC9 | 18.48 | 5.04e−05 | 8.53e−02 | trinucleotide repeat containing 9 | 16q11.2 | Consensussequence | Hs.110826.1 |
| 34 | 243953_at | FADS2 | 18.44 | 4.81e−05 | 8.46e−02 | fatty acid desaturase 2 | 11q12-q13.1 | Consensussequence | Hs.100848.0 |
| 35 | 239072_at | | 18.32 | 7.77e−05 | 9.37e−02 | Human S6 H-8 mRNA expressed in | | Consensussequence | Hs.212529.0 |
| | | | | | | chromosome 6-suppressed melanoma | | | |
| | | | | | | cells. | | | |
| 36 | 206615_s_at | ADAM22 | 18.30 | 1.81e−04 | 0.12 | a disintegrin and metalloproteinase | 7q21 | Exemplarsequence | Hs.158052.1 |
| | | | | | | domain 22 | | | |
| 37 | 207493_x_at | SSX2 | 18.09 | 6.05e−05 | 8.62e−02 | synovial sarcoma, X breakpoint 2 | Xp11.23-p11.22 | Exemplarsequence | Hs.289105.0 |
| 38 | 239334_at | | 18.00 | 9.96e−05 | 0.10 | ESTs | | Consensussequence | Hs.172762.0 |
| 39 | 200608_s_at | RAD21 | 18.00 | 2.51e−04 | 0.14 | RAD21 homolog (S. pombe) | 8q24 | Exemplarsequence | Hs.81848.0 |
| 40 | 204739_at | CENPC1 | 17.97 | 3.08e−04 | 0.16 | centromere protein C 1 | 4q12-q13.3 | Exemplarsequence | Hs.154207.0 |
| 41 | 230210_at | UNC84A | 17.62 | 6.96e−05 | 9.27e−02 | unc-84 homolog A (C. elegans) | 7p22.3 | Consensussequence | Hs.7531.3 |
| 42 | 241172_at | GRIA2 | 17.55 | 7.27e−05 | 9.37e−02 | glutamate receptor, ionotropic, AMPA 2 | 4q32-q33 | Consensussequence | Hs.161378.0 |
| 43 | 216023_at | KIAA0876 | 17.53 | 1.04e−04 | 0.10 | KIAA0876 protein | 19p13.3 | Consensussequence | Hs.306827.0 |
| 44 | 227762_at | | 17.41 | 1.06e−03 | 0.24 | ESTs | | Consensussequence | Hs.22971.0 |
| 45 | 238933_at | IRS1 | 17.36 | 9.84e−05 | 0.10 | insulin receptor substrate 1 | 2q36 | Consensussequence | Hs.59896.0 |
| 46 | 232378_at | LOC200010 | 17.36 | 1.62e−04 | 0.12 | hypothetical protein LOC200010 | 1p33 | Consensussequence | Hs.37890.0 |
| 47 | 238358_x_at | | 17.28 | 7.88e−05 | 9.37e−02 | ESTs | | Consensussequence | Hs.145492.0 |
| 48 | 243359_at | | 17.13 | 1.16e−04 | 0.10 | ESTs | | Consensussequence | Hs.202388.0 |
| 49 | 216749_at | | 17.02 | 9.42e−05 | 0.10 | Homo sapiens cDNA: FLJ21198 fis, | | Consensussequence | Hs.306707.0 |
| | | | | | | clone COL00220. | | | |
| 50 | 235874_at | MGC46520 | 17.01 | 1.28e−04 | 0.11 | hypothetical protein MGC46520 | 6q14.3 | Consensussequence | Hs.98381.0 |

| | Sequence | | Sequence | | | | |
|---|---|---|---|---|---|---|---|
| # | Derived From | Sequence ID | Source | Unigene_Accession | Cluster_Type | LocusLink | Full_Length_Reference_Seq |
| 1 | AI680723 | Hs.65919.1_RC | GenBank | Hs.53542 | fulllength | 23230 | NM_015186; chorein isoform B NM_033305; chorein isoform A |
| 2 | BE219961 | Hs.109804.1_RC | GenBank | Hs.75307 | fulllength | 7052 | NM_004613; transglutaminase 2 (C polypeptide, protein-glutamine-gamma- |
| | | | | | | | glutamyltransferase) |
| 3 | BG168896 | Hs.138381.1.A1 | GenBank | Hs.356463 | fulllength | 2339 | NM_002027; farnesyltransferase, CAAX box, alpha |
| 4 | NM_020653.1 | g10190687 | RefSeq | Hs.99724 | fulllength | 57336 | NM_020653; zinc finger protein 287 |
| 5 | AW297087 | Hs.92025.0 | GenBank | Hs.437611 | est | | |
| 6 | NM_005567.2 | g6006016 | RefSeq | Hs.79339 | fulllength | 3959 | NM_005567; galectin 3 binding protein |
| 7 | BF110180 | Hs.117853.0 | GenBank | Hs.117853 | | | |
| 8 | AA947302 | Hs.28714.0.A1 | GenBank | Hs.46700 | fulllength | 3621 | NM_005537; inhibitor of growth family, member 1 |
| 9 | AU143984 | Hs.301645.0 | GenBank | Hs.397840 | | | |
| 10 | AA443280 | Hs.98995.0.A1 | GenBank | Hs.279907 | fulllength | 53904 | NM_017433; myosin IIIA |
| 11 | H08345 | Hs.106234.0.A1 | GenBank | Hs.106234 | | | |
| 12 | AI306990 | Hs.133292.0 | GenBank | Hs.432435 | est | | |
| 13 | AW134979 | Hs.99291.1.S1 | GenBank | Hs.99291 | fulllength | 29091 | NM_014178; amisyn |
| 14 | NM_005176.3 | g6671590 | RefSeq | Hs.89399 | fulllength | 517 | NM_005176; ATP synthase, H+ transporting, mitochondrial F0 complex, subunit c |
| | | | | | | | (subunit 9), isoform 2 |
| 15 | AI990286 | Hs.147260.0_RC | GenBank | Hs.147260 | est | | |
| 16 | BF590323 | Hs.55256.0.A1 | GenBank | Hs.101660 | fulllength | 84826 | NM_032740; hypothetical protein MGC5391 |
| 17 | U13216.1 | g532741 | GenBank | Hs.170917 | fulllength | 5733 | NM_000957; prostaglandin E receptor 3 (subtype EP3) |
| 18 | BE676209 | Hs.80475.2.S1 | GenBank | Hs.375569 | fulllength | 246721 | NM_032958; DNA directed RNA polymerase II polypeptide J-related gene isoform 2 |
| | | | | | | | NM_032959; DNA directed RNA polymerase II polypeptide J-related gene isoform 3 |
| | | | | | | | NM_145325; DNA directed RNA polymerase II polypeptide J-related gene isoform 1 |
| 19 | BE613520 | Hs.120654.0_RC | GenBank | Hs.120654 | | | |
| 20 | AF248966.1 | g12005668 | GenBank | Hs.183434 | fulllength | 10159 | NM_005765; ATPase, H+ transporting, lysosomal, membrane sector associated |
| | | | | | | | protein M8-9 |
| 21 | BG399778 | Hs.293970.0.A2 | GenBank | Hs.293970 | fulllength | 4329 | NM_005589; aldehyde dehydrogenase 6A1 precursor |
| 22 | AW300309 | Hs.27453.0 | GenBank | Hs.27453 | fulllength | 142684 | NM_080879; RAB40A, member RAS oncogene family |
| 23 | NM_014793.1 | g7662173 | RefSeq | Hs.200596 | fulllength | 9836 | NM_014793; p21WAF1/CIP1 promoter-interacting protein |
| 24 | NM_006280.1 | g5454089 | RefSeq | Hs.102135 | fulllength | 6748 | NM_006280; signal sequence receptor, delta |
| 25 | NM_002112.1 | g4504364 | RefSeq | Hs.1481 | fulllength | 3067 | NM_002112; histidine decarboxylase |
| 26 | R92115 | Hs.117900.0.A1 | GenBank | Hs.444881 | est | | |
| 27 | NM_003834.1 | g4506506 | RefSeq | Hs.65756 | fulllength | 8786 | NM_003834; regulator of G-protein signalling 11 |
| 28 | NM_002500.1 | g4505376 | RefSeq | Hs.72981 | fulllength | 4760 | NM_002500; neurogenic differentiation 1 |
| 29 | AV705233 | Hs.132678.0.A1 | GenBank | Hs.132678 | est | | |
| 30 | AU146809 | Hs.287476.0 | GenBank | Hs.355194 | | | |
| 31 | AI912612 | Hs.213049.0.A1 | GenBank | Hs.213049 | est | | |
| 32 | W42591 | Hs.23892.0.A1 | GenBank | Hs.436562 | est | | |
| 33 | U80736.1 | Hs.110826.1 | GenBank | Hs.110826 | | 27324 | |
| 34 | R40016 | Hs.100848.0_RC | GenBank | Hs.184641 | est | 9415 | NM_004265; fatty acid desaturase 2 |
| 35 | BE080109 | Hs.212529.0.A1 | GenBank | Hs.446408 | | | |
| 36 | NM_021723.1 | g11497046 | RefSeq | Hs.158052 | fulllength | 53616 | NM_004194; a disintegrin and metalloproteinase domain 22 isoform 4 proprotein |
| | | | | | | | NM_016351; a disintegrin and metalloproteinase domain 22 isoform 3 proprotein |
| | | | | | | | NM_021721; a disintegrin and metalloproteinase domain 22 isoform 5 proprotein |
| | | | | | | | NM_021722; a disintegrin and metalloproteinase domain 22 isoform 2 proprotein |
| | | | | | | | NM_021723; a disintegrin and metalloproteinase domain 22 isoform 1 proprotein |
| 37 | NM_003147.1 | g10337582 | RefSeq | Hs.446305 | fulllength | 6757 | NM_003147; synovial sarcoma, X breakpoint 2 isoform a NM_175698; synovial |
| | | | | | | | sarcoma, X breakpoint 2 isoform b |
| 38 | AI349167 | Hs.172762.0 | GenBank | Hs.172762 | est | | |
| 39 | NM_006265.1 | g5453993 | RefSeq | Hs.81848 | fulllength | 5885 | NM_006265; RAD21 homolog |
| 40 | NM_001812.1 | g4502778 | RefSeq | Hs.154207 | fulllength | 1060 | NM_001812; centromere protein C 1 |
| 41 | AW054783 | Hs.7531.3.S1 | GenBank | Hs.381143 | fulllength | 23353 | |
| 42 | AI939470 | Hs.161378.0_RC | GenBank | Hs.89582 | fulllength | 2891 | NM_000826; glutamate receptor, ionotropic, AMPA 2 |
| 43 | AK026040.1 | Hs.306827.0 | GenBank | Hs.301011 | | 23030 | |
| 44 | AW244016 | Hs.22971.0.A1 | GenBank | Hs.356349 | est | | |
| 45 | AA644178 | Hs.59896.0_RC | GenBank | Hs.96063 | fulllength | 3667 | NM_005544; insulin receptor substrate 1 |
| 46 | AI767388 | Hs.37890.0.S1 | GenBank | Hs.37890 | | 200010 | |
| 47 | AI366965 | Hs.145492.0_RC | GenBank | Hs.432397 | est | | |
| 48 | AI701857 | Hs.202388.0_RC | GenBank | Hs.202388 | est | | |
| 49 | AK024851.1 | Hs.306707.0 | GenBank | Hs.306707 | | | |
| 50 | AL574912 | Hs.98381.0_RC | GenBank | Hs.98368 | fulllength | 167681 | NM_153362; hypothetical protein MGC46520 |

TABLE 34
_complex

| # | affy id | HUGO name | Title | MapLocation | Sequence Type | Transcript ID |
|---|---|---|---|---|---|---|
| 1 | 222551_s_at | FLJ20989 | hypothetical protein FLJ20989 | | Consensussequence | Hs.169615.0 |
| 2 | 226754_at | LOC90987 | hypothetical protein BC006258 | 8q24.3 | Consensussequence | Hs.285901.0 |
| 3 | 206573_at | KCNQ3 | potassium voltage-gated channel, KQT-like subfamily, member 3 | 8q24 | Exemplarsequence | Hs.40866.0 |
| 4 | 225439_at | CML66 | chronic myelogenous leukemia tumor antigen 66 | 8q23 | Consensussequence | Hs.195870.0 |
| 5 | 225676_s_at | DKFZP564O0463 | DKFZP564O0463 protein | 8q22.3 | Consensussequence | Hs.273344.1 |
| 6 | 200949_x_at | RPS20 | ribosomal protein S20 | 8q12 | Exemplarsequence | Hs.8102.0 |
| 7 | 210397_at | DEFB1 | defensin, beta 1 | 8p23.2-p23.1 | Exemplarsequence | Hs.32949.0 |
| 8 | 208423_s_at | MSR1 | macrophage scavenger receptor 1 | 8p22 | Exemplarsequence | Hs.49.0 |
| 9 | 222525_s_at | FLJ10853 | hypothetical protein FLJ10853 | 8p21.1 | Consensussequence | Hs.72085.0 |
| 10 | 231961_at | | Homo sapiens cDNA FLJ14130 fis, clone MAMMA1002618 | | Consensussequence | Hs.112572.0 |
| 11 | 224127_at | C8orf4 | chromosome 8 open reading frame 4 | 8p11.2 | Exemplarsequence | Hs.31446.0 |
| 12 | 205089_at | ZNF7 | zinc finger protein 7 (KOX 4, clone HF.16) | 8q24 | Exemplarsequence | Hs.2076.0 |
| 13 | 224387_at | HT002 | HT002 protein; hypertension-related calcium-regulated gene | 8q24-qter | Exemplarsequence | g9964286 |
| 14 | 218187_s_at | FLJ20989 | hypothetical protein FLJ20989 | | Exemplarsequence | Hs.169615.0 |
| 15 | 222998_at | MAF1 | homolog of yeast MAF1 | 8q24.3 | Exemplarsequence | Hs.324157.0 |
| 16 | 218679_s_at | VPS28 | vacuolar protein sorting 28 (yeast) | 8q24.3 | Exemplarsequence | Hs.293441.0 |
| 17 | 202344_at | HSF1 | heat shock transcription factor 1 | 8q24.3 | Exemplarsequence | Hs.1499.0 |
| 18 | 201066_at | CYC1 | cytochrome c-1 | 8q24.3 | Exemplarsequence | Hs.289271.0 |
| 19 | 229758_at | TIGD5 | tigger transposable element derived 5 | 8q24.3 | Consensussequence | Hs.71574.0 |
| 20 | 225949_at | | Homo sapiens mRNA; cDNA DKFZp434P086 (from clone DKFZp434P086); | | Consensussequence | Hs.274401.0 |
| | | | partial cds | | | |
| 21 | 209899_s_at | SIAHBP1 | fuse-binding protein-interacting repressor | 8q24.2-qtel | Exemplarsequence | Hs.74562.0 |
| 22 | 33132_at | CPSF1 | cleavage and polyadenylation specific factor 1, 160 kDa | 8q24.23 | Consensussequence | 8 |
| 23 | 217534_at | | | | Consensussequence | Hs.282721.0 |
| 24 | 229802_at | | Homo sapiens cDNA FLJ14388 fis, clone HEMBA1002716. | | Consensussequence | Hs.9812.0 |
| 25 | 223231_at | CDA11 | CDA11 protein | 8q24.13 | Exemplarsequence | Hs.11810.0 |
| 26 | 241342_at | LOC157378 | hypothetical protein BC017881 | 8q24.13 | Consensussequence | Hs.187646.0 |
| 27 | 238741_at | | Homo sapiens cDNA FLJ25541 fis, clone JTH00915. | | Consensussequence | Hs.193341.0 |
| 28 | 224218_s_at | TRPS1 | trichorhinophalangeal syndrome I | 8q24.12 | Exemplarsequence | Hs.26102.1 |
| 29 | 209523_at | TAF2 | TAF2 RNA polymerase II, TATA box binding protein (TBP)-associated factor, | 8q24.12 | Consensussequence | Hs.122752.0 |
| | | | 150 kDa | | | |
| 30 | 227402_s_at | MGC14595 | hypothetical protein MGC14595 | 8q23.3 | Consensussequence | Hs.323662.0 |
| 31 | 200608_s_at | RAD21 | RAD21 homolog (S. pombe) | 8q24 | Exemplarsequence | Hs.81848.0 |
| 32 | 218692_at | FLJ20366 | hypothetical protein FLJ20366 | 8q23.2 | Exemplarsequence | Hs.8358.0 |
| 33 | 204278_s_at | EBAG9 | estrogen receptor binding site associated, antigen, 9 | 8q23 | Exemplarsequence | Hs.9222.0 |
| 34 | 218059_at | LOC51123 | HSPC038 protein | 8q22.3 | Exemplarsequence | Hs.23528.0 |
| 35 | 204068_at | STK3 | serine/threonine kinase 3 (STE20 homolog, yeast) | 8q22.1 | Exemplarsequence | Hs.166684.0 |
| 36 | 213243_at | KIAA0532 | KIAA0532 protein | 8q22.2 | Consensussequence | Hs.21077.1 |
| 37 | 230016_at | | Homo sapiens cDNA FLJ13277 fis, clone OVARC1001044. | | Consensussequence | Hs.55043.0 |
| 38 | 213122_at | KIAA1750 | KIAA1750 protein | 8q22.1 | Consensussequence | Hs.173094.0 |
| 39 | 201433_s_at | PTDSS1 | phosphatidylserine synthase 1 | 8q22 | Exemplarsequence | Hs.77329.0 |
| 40 | 208882_s_at | DD5 | progestin induced protein | 8q22 | Consensussequence | Hs.278428.0 |
| 41 | 228047_at | RPL30 | ribosomal protein L30 | 8q22 | Consensussequence | Hs.111222.1 |
| 42 | 225912_at | TP53INP1 | tumor protein p53 inducible nuclear protein 1 | 8q22 | Consensussequence | Hs.75497.0 |
| 43 | 212637_s_at | WWP1 | WW domain-containing protein 1 | 8q21 | Consensussequence | Hs.324275.0 |
| 44 | 204865_at | CA3 | carbonic anhydrase III, muscle specific | 8q13-q22 | Exemplarsequence | Hs.82129.0 |
| 45 | 235491_at | RINZF | zinc finger protein RINZF | 8q13-q21.1 | Consensussequence | Hs.194136.0 |
| 46 | 206584_at | LY96 | lymphocyte antigen 96 | 8q13.3 | Exemplarsequence | Hs.69328.0 |
| 47 | 226271_at | | Homo sapiens mRNA; cDNA DKFZp566A1046 (from clone | | Consensussequence | Hs.168950.0 |
| | | | DKFZp566A1046) | | | |
| 48 | 201398_s_at | TRAM | translocating chain-associating membrane protein | 8q13.1 | Exemplarsequence | Hs.4147.0 |
| 49 | 215605_at | | | | Consensussequence | Hs.296664.0 |
| 50 | 221925_s_at | FLJ22490 | hypothetical protein FLJ22490 | 8q12.3 | Consensussequence | Hs.153746.1 |
| 51 | 227105_at | FLJ22490 | hypothetical protein FLJ22490 | 8q12.3 | Consensussequence | Hs.153746.1 |
| 52 | 203269_at | NSMAF | neutral sphingomyelinase (N-SMase) activation associated factor | 8q12-q13 | Exemplarsequence | Hs.78687.0 |
| 53 | 238017_at | RDH-E2 | retinal short chain dehydrogenase reductase | 8q12.1 | Consensussequence | Hs.170673.0 |
| 54 | 220619_at | KIAA1416 | KIAA1416 protein | 8q12.1 | Exemplarsequence | Hs.105461.1 |
| 55 | 216246_at | RPS20 | ribosomal protein S20 | 8q12 | Consensussequence | Hs.8102.2 |
| 56 | 218027_at | MRPL15 | mitochondrial ribosomal protein L15 | 8q11.2-q13 | Exemplarsequence | Hs.18349.0 |
| 57 | 224743_at | | Homo sapiens, clone IMAGE: 3897094, mRNA | | Consensussequence | Hs.13328.0 |
| 58 | 226483_at | FLJ32370 | hypothetical protein FLJ32370 | 8q11.23 | Consensussequence | Hs.280858.1 |
| 59 | 212142_at | MCM4 | MCM4 minichromosome maintenance deficient 4 (S. cerevisiae) | 8q12-q13 | Consensussequence | Hs.154443.0 |
| 60 | 219231_at | NCOA6IP | nuclear receptor coactivator 6 interacting protein | 8q11 | Exemplarsequence | Hs.179909.0 |
| 61 | 221367_at | MOS | v-mos Moloney murine sarcoma viral oncogene homolog | 8q11 | Exemplarsequence | Hs.248146.0 |
| 62 | 233101_at | | Homo sapiens cDNA FLJ12009 fis, clone HEMBB1001618. | | Consensussequence | Hs.287478.0 |
| 63 | 227017_at | LOC157697 | hypothetical protein LOC157697 | 8p23.3 | Consensussequence | Hs.193090.0 |
| 64 | 242387_at | INM01 | hypothetical protein INM01 | 8p23.3 | Consensussequence | Hs.289293.0 |
| 65 | 208647_at | FDFT1 | farnesyl-diphosphate farnesyltransferase 1 | 8p23.1-p22 | Consensussequence | Hs.48876.1 |
| 66 | 210950_s_at | FDFT1 | farnesyl-diphosphate farnesyltransferase 1 | 8p23.1-p22 | Exemplarsequence | Hs.48876.0 |
| 67 | 218096_at | FLJ11210 | hypothetical protein FLJ11210 | 8p23.1 | Exemplarsequence | Hs.27842.0 |
| 68 | 236034_at | | ESTs | | Consensussequence | Hs.68301.0 |
| 69 | 205572_at | ANGPT2 | angiopoietin 2 | 8p23.1 | Exemplarsequence | Hs.115181.0 |
| 70 | 200090_at - HG-U133A | FNTA | farnesyltransferase, CAAX box, alpha | 8p22-q11 | Consensussequence | Hs.138381.1 |
| 71 | 209471_s_at | FNTA | farnesyltransferase, CAAX box, alpha | 8p22-q11 | Exemplarsequence | Hs.138381.0 |
| 72 | 200090_at - HG-U133B | FNTA | farnesyltransferase, CAAX box, alpha | 8p22-q11 | Consensussequence | Hs.138381.1 |
| 73 | 225053_at | CNOT7 | CCR4-NOT transcription complex, subunit 7 | 8p22-p21.3 | Consensussequence | Hs.226318.1 |
| 74 | 218250_s_at | CNOT7 | CCR4-NOT transcription complex, subunit 7 | 8p22-p21.3 | Exemplarsequence | Hs.226318.0 |
| 75 | 202174_s_at | PCM1 | pericentriolar material 1 | 8p22-p21.3 | Exemplarsequence | Hs.75737.0 |
| 76 | 214118_x_at | PCM1 | pericentriolar material 1 | 8p22-p21.3 | Consensussequence | Hs.75737.1 |
| 77 | 214937_x_at | PCM1 | pericentriolar material 1 | 8p22-p21.3 | Consensussequence | Hs.75737.2 |
| 78 | 233019_at | | Homo sapiens cDNA FLJ13499 fis, clone PLACE1004681. | | Consensussequence | Hs.287574.0 |
| 79 | 201089_at | ATP6V1B2 | ATPase, H+ transporting, lysosomal 56/58 kDa, V1 subunit B, | 8p22-p21 | Exemplarsequence | Hs.1697.0 |
| | | | isoform 2 | | | |
| 80 | 225378_at | FLJ32642 | hypothetical protein FLJ32642 | 8p22 | Consensussequence | Hs.101617.0 |
| 81 | 228024_at | FLJ32642 | hypothetical protein FLJ32642 | 8p22 | Consensussequence | Hs.290855.0 |
| 82 | 201985_at | KIAA0196 | KIAA0196 gene product | 8p22 | Exemplarsequence | Hs.8294.0 |
| 83 | 209228_x_at | N33 | Putative prostate cancer tumor suppressor | 8p22 | Exemplarsequence | Hs.71119.0 |
| 84 | 218337_at | RAI16 | retinoic acid induced 16 | 8p21.2 | Exemplarsequence | Hs.299148.0 |
| 85 | 214054_at | DOK2 | docking protein 2, 56 kDa | 8p21.2 | Consensussequence | Hs.71215.0 |
| 86 | 222199_s_at | BIN3 | bridging integrator 3 | 8p21.2 | Consensussequence | Hs.68090.1 |
| 87 | 207000_s_at | PPP3CC | protein phosphatase 3 (formerly 2B), catalytic subunit, gamma | 8p21.2 | Exemplarsequence | Hs.75206.0 |
| | | | isoform (calcineurin A gamma) | | | |
| 88 | 227263_at | FLJ34715 | hypothetical protein FLJ34715 | 8p21.2 | Consensussequence | Hs.296360.0 |
| 89 | 209253_at | SCAM-1 | vinexin beta (SH3-containing adaptor molecule-1) | 8p21.2 | Exemplarsequence | Hs.33787.1 |
| 90 | 32541_at | PPP3CC | protein phosphatase 3 (formerly 2B), catalytic subunit, gamma | 8p21.2 | Consensussequence | 6 |
| | | | isoform (calcineurin A gamma) | | | |
| 91 | 206134_at | ADAMDEC1 | ADAM-like, decysin 1 | 8p21.1 | Exemplarsequence | Hs.145296.0 |
| 92 | 232693_s_at | LOC55893 | papillomavirus regulatory factor PRF-1 | 8p21.1 | Consensussequence | Hs.27410.2 |
| 93 | 223216_x_at | LOC55893 | papillomavirus regulatory factor PRF-1 | 8p21.1 | Exemplarsequence | Hs.27410.1 |
| 94 | 227075_at | ELP3 | likely ortholog of mouse elongation protein 3 homolog (S. cerevisiae) | 8p21.1 | Consensussequence | Hs.267905.2 |
| 95 | 202962_at | KIF13B | kinesin family member 13B | 8p12 | Exemplarsequence | Hs.15711.0 |
| 96 | 228427_at | FBX16 | F-box protein 16 | 8p21.1 | Consensussequence | Hs.135191.0 |
| 97 | 219269_at | FLJ21616 | hypothetical protein FLJ21616 | 8p12 | Exemplarsequence | Hs.14562.0 |
| 98 | 236812_at | | ESTs | | Consensussequence | Hs.223267.0 |
| 99 | 221916_at | NEFL | neurofilament, light polypeptide 68 kDa | 8p21 | Consensussequence | Hs.302689.1 |
| 100 | 207595_s_at | BMP1 | bone morphogenetic protein 1 | 8p21 | Exemplarsequence | Hs.1274.3 |
| 101 | 240168_at | | ESTs, Moderately similar to RAN binding protein 16 [Homo | | Consensussequence | Hs.308435.0 |
| | | | sapiens] [H. sapiens] | | | |
| 102 | 201375_s_at | PPP2CB | protein phosphatase 2 (formerly 2A), catalytic subunit, beta isoform | 8p12-p11.2 | Exemplarsequence | Hs.80350.0 |
| 103 | 204015_s_at | DUSP4 | dual specificity phosphatase 4 | 8p12-p11 | Exemplarsequence | Hs.2359.0 |
| 104 | 207836_s_at | RBPMS | RNA binding protein with multiple splicing | 8p12-p11 | Exemplarsequence | Hs.80248.0 |
| 105 | 235472_at | FUT10 | fucosyltransferase 10 (alpha (1,3) fucosyltransferase) | 8p11.23 | Consensussequence | Hs.132665.0 |
| 106 | 221539_at | EIF4EBP1 | eukaryotic translation initiation factor 4E binding protein 1 | 8p12 | Exemplarsequence | Hs.71819.0 |
| 107 | 207937_x_at | FGFR1 | fibroblast growth factor receptor 1 (fms-related tyrosine kinase 2, | 8p11.2-p11.1 | Exemplarsequence | Hs.748.2 |
| | | | Pfeiffer syndrome) | | | |
| 108 | 212690_at | KIAA0725 | KIAA0725 protein | 8p11.21 | Consensussequence | Hs.26450.0 |
| 109 | 225318_at | | Homo sapiens cDNA: FLJ22049 fis, clone HEP09444. | | Consensussequence | Hs.28274.0 |
| 110 | 224413_s_at | BLP1 | BBP-like protein 1 | 8p11.21 | Exemplarsequence | g13625460 |
| 111 | 225534_at | LOC114926 | hypothetical protein BC013035 | 8p11.1 | Consensussequence | Hs.10018.1 |
| 112 | 222544_s_at | WHSC1L1 | Wolf-Hirschhorn syndrome candidate 1-like 1 | 8p11.2 | Consensussequence | Hs.27721.0 |
| 113 | 209384_at | PROSC | proline synthetase co-transcribed homolog (bacterial) | 8p11.2 | Consensussequence | Hs.301959.0 |
| 114 | 203616_at | POLB | polymerase (DNA directed), beta | 8p11.2 | Exemplarsequence | Hs.180107.0 |
| 115 | 224076_s_at | WHSC1L1 | Wolf-Hirschhorn syndrome candidate 1-like 1 | 8p11.2 | Exemplarsequence | Hs.27721.1 |
| 116 | 218541_s_at | C8orf4 | chromosome 8 open reading frame 4 | 8p11.2 | Exemplarsequence | Hs.283683.0 |
| 117 | 218017_s_at | FLJ22242 | hypothetical protein FLJ22242 | 8p11.1 | Exemplarsequence | Hs.288057.0 |
| 118 | 205528_s_at | CBFA2T1 | core-binding factor, runt domain, alpha subunit 2; translocated to, | 8q22 | Consensussequence | Hs.31551.0 |
| | | | 1; cyclin D-related | | | |
| 119 | 228827_at | | Homo sapiens clone 25023 mRNA sequence | | Consensussequence | Hs.90858.0 |
| 120 | 213423_x_at | N33 | Putative prostate cancer tumor suppressor | 8p22 | Consensussequence | Hs.71119.1 |

| # | Sequence Derived From | Sequence ID | Sequence Source | Unigene_Accession | Cluster_Type | LocusLink | Full_Length_Reference_Seq |
|---|---|---|---|---|---|---|---|
| 1 | AI197841 | Hs.169615.0 | GenBank | Hs.169615 | fulllength | 65265 | NM_023080; hypothetical protein FLJ20989 |
| 2 | W93231 | Hs.285901.0 | GenBank | Hs.285901 | | 90987 | |
| 3 | NM_004519.1 | g4758629 | RefSeq | Hs.40866 | fulllength | 3786 | NM_004519; potassium voltage-gated channel KQT-like protein 3 |
| 4 | BC000967.2 | Hs.195870.0 | GenBank | Hs.195870 | fulllength | 84955 | NM_032869; chronic myelogenous leukemia tumor antigen 66 |
| 5 | AK001693.1 | Hs.273344.1 | GenBank | Hs.273344 | fulllength | 25879 | NM_014156; NM_015420; DKFZP564O0463 protein |
| 6 | NM_001023.1 | g4506696 | RefSeq | Hs.8102 | fulllength | 6224 | NM_001023; ribosomal protein S20 |
| 7 | U73945.1 | g1755147 | GenBank | Hs.32949 | fulllength | 1672 | NM_005218; defensin, beta 1, preproprotein |
| 8 | NM_002445.1 | g4505258 | RefSeq | Hs.49 | fulllength | 4481 | NM_002445; macrophage scavenger receptor 1 isoform type 2 |
| | | | | | | | NM_138715; macrophage scavenger receptor 1 isoform type 1 |
| | | | | | | | NM_138716; macrophage scavenger receptor 1 isoform type 3 |
| 9 | AU160632 | Hs.72085.0 | GenBank | Hs.72085 | fulllength | 55246 | NM_018246; hypothetical protein FLJ10853 |
| 10 | AU148114 | Hs.112572.0.S1 | GenBank | Hs.112572 | | | |
| 11 | AF116660.1 | g7959818_RC | GenBank | Hs.283683 | fulllength | 56892 | NM_020130; chromosome 8 open reading frame 4 |
| 12 | NM_003416.1 | g4508034 | RefSeq | Hs.2076 | fulllength | 7553 | NM_003416; zinc finger protein 7 (KOX 4, clone HF.16) |
| 13 | AF290195.1 | g9964286 | GenBank | Hs.238928 | fulllength | 28991 | NM_014066; hypertension-related calcium-regulated gene |
| 14 | NM_023080.1 | g12751496 | RefSeq | Hs.169615 | fulllength | 65265 | NM_023080; hypothetical protein FLJ20989 |
| 15 | AL136937.1 | g12053368 | GenBank | Hs.19673 | fulllength | 84232 | NM_032272; homolog of yeast MAF1 |
| 16 | NM_016208.1 | g7705884 | RefSeq | Hs.339697 | fulllength | 51160 | NM_016208; VPS28 protein |
| 17 | NM_005526.1 | g5031766 | RefSeq | Hs.380935 | fulllength | 3297 | NM_005526; heat shock transcription factor 1 |
| 18 | NM_001916.1 | g4503184 | RefSeq | Hs.289271 | fulllength | 1537 | NM_001916; cytochrome c-1 |
| 19 | AW168771 | Hs.71574.0 | GenBank | Hs.71574 | fulllength | 84948 | NM_032862; tigger transposable element derived 5 |
| 20 | N21030 | Hs.274401.0.S1 | GenBank | Hs.274401 | fulllength | | |
| 21 | AF217197.1 | g6740005 | GenBank | Hs.74562 | fulllength | 22827 | NM_014281; fuse-binding protein-interacting repressor isoform b |
| | | | | | | | NM_078480; fuse-binding protein-interacting repressor isoform a |
| 22 | U37012 | 4923232 | GenBank | Hs.83727 | fulllength | 29894 | NM_013291; cleavage and polyadenylation specific factor 1, 160 kDa |
| 23 | AV720514 | Hs.282721.0 | GenBank | | | | |
| 24 | AA147884 | Hs.9812.0.A1 | GenBank | Hs.9812 | | | |
| 25 | AF212250.1 | g13182774 | GenBank | Hs.11810 | fulllength | 83940 | NM_032026; CDA11 protein |
| 26 | BG288115 | Hs.187646.0.A1 | GenBank | Hs.187646 | fulllength | 157378 | |
| 27 | BE157240 | Hs.193341.0_RC | GenBank | Hs.379094 | | | |
| 28 | AF264784.1 | g10644121 | GenBank | Hs.26102 | fulllength | 7227 | NM_014112; zinc finger transcription factor TRPS1 |
| 29 | AK001618.1 | Hs.122752.0 | GenBank | Hs.122752 | fulllength | 6873 | NM_003184; TBP-associated factor 2 |
| 30 | AI056895 | Hs.323662.0.A1 | GenBank | Hs.323662 | fulllength | 84294 | NM_032334; hypothetical protein MGC14595 |
| 31 | NM_006265.1 | g5453993 | RefSeq | Hs.81848 | fulllength | 5885 | NM_006285: RAD21 homotog |
| 32 | NM_017786.1 | g8923340 | RefSeq | Hs.8358 | fulllength | 55638 | NM_017786; hypothetical protein FLJ20366 |
| 33 | NM_004215.1 | g4758229 | RefSeq | Hs.9222 | fulllength | 9166 | NM_004215: estrogen receptor binding site associated antigen 9 |
| 34 | NM_016096.1 | g7705516 | RefSeq | Hs.23528 | fulllength | 51123 | NM_016096; HSPC038 protein |
| 35 | NM_006281.1 | g5454093 | RefSeq | Hs.166684 | fulllength | 6788 | NM_006281; serine/threonine kinase 3 (STE20 homolog, yeast) |
| 36 | AI052003 | Hs.21077.1.A1 | GenBank | Hs.21077 | | 23292 | |
| 37 | AU155118 | Hs.55043.0.S1 | GenBank | Hs.55043 | | | |
| 38 | AI096375 | Hs.173094.0.S1 | GenBank | Hs.173094 | fulllength | 85453 | NM_033512; KIAA1750 protein |
| 39 | NM_014754.1 | g7662646 | RefSeq | Hs.77329 | fulllength | 9791 | NM_014754; phosphatidylserine synthase 1 |
| 40 | U69567 | Hs.278428.0.A2 | GenBank | Hs.278428 | fulllength | 51366 | NM_015902; progestin induced protein |
| 41 | AI815001 | Hs.111222.1 | GenBank | Hs.334807 | fulllength | 6156 | NM_000989; ribosomal protein L30 |
| 42 | AW341649 | Hs.75497.0.A1 | GenBank | Hs.75497 | fulllength | 94241 | NM_033285; tumor protein p53 inducible nuclear protein 1 |
| 43 | BF131791 | Hs.324275.0.S1 | GenBank | Hs.355977 | fulllength | 11059 | NM_007013; WW domain-containing protein 1 |
| 44 | NM_005181.2 | g6996001 | RefSeq | Hs.82129 | fulllength | 761 | NM_005181; carbonic anhydrase III |
| 45 | BF056208 | Hs.194136.0_RC | GenBank | Hs.237146 | fulllength | 65986 | NM_023929; zinc finger protein RINZF |
| 46 | NM_015364.1 | g7662503 | RefSeq | Hs.69328 | fulllength | 23643 | NM_015364; MD-2 protein |
| 47 | AL110252.1 | Hs.168950.0 | GenBank | Hs.168950 | | | |
| 48 | BC000687.1 | g12653796 | GenBank | Hs.4147 | fulllength | 23471 | NM_014294; translocating chain-associating membrane protein |
| 49 | AU145806 | Hs.296664.0 | GenBank | | | | |
| 50 | BE044503 | Hs.153746.1.S1 | GenBank | Hs.153746 | fulllength | 79848 | NM_024790; hypothetical protein FLJ22490 |
| 51 | AI400587 | Hs.153746.1.S2 | GenBank | Hs.153746 | fulllength | 79848 | NM_024790; hypothetical protein FLJ22490 |
| 52 | NM_003580.1 | g4505464 | RefSeq | Ns.78687 | fulllength | 8439 | NM_003580; neutral sphingomyetinase (N-SMase) activation associated |
| | | | | | | | factor. |
| 53 | AI440266 | Hs.170673.0.A1 | GenBank | Hs.170873 | fulllength | 195814 | NM_138969; retinal short chain dehydrogenase reductase |
| 54 | NM_017783.1 | g11034826 | RefSeq | Hs.105461 | | 55636 | |
| 55 | AF113008.1 | Hs.8102.2 | GenBank | Hs.8102 | fulllength | 6224 | NM_001023; ribosomal protein S20 |
| 56 | NM_014175.1 | g7661805 | RefSeq | Hs.18349 | fulllength | 29088 | NM_014175; mitochondrial ribosomal protein L15 |
| 57 | BF965065 | Hs.13328.0_RC | GenBank | Hs.416904 | | | |
| 58 | AI890761 | Hs.280858.1.A1 | GenBank | Hs.406141 | fulllength | 137695 | NM_152417; hypothetical protein FLJ32370 |
| 59 | X74794.1 | Hs.154443.0 | GenBank | Hs.154443 | fulllength | 4173 | |
| 60 | NM_024831.1 | p13376235 | RefSeq | Hs.179909 | fulllength | 96764 | NM_024831: PRIP-interacting protein PIPMT |
| 61 | NM_005372.1 | g4885488 | RefSeq | Hs.248146 | fulllength | 4342 | NM_005372: v-mos Moloney murine sarcoma viral oncogene homolog |
| 62 | AK022071.1 | Hs.287478.0 | GenBank | Hs.287478 | | | |
| 63 | AA767385 | Hs.193090.0.A1 | GenBank | Hs.377879 | fulllength | 157697 | |
| 64 | BF509686 | Hs.289293.0 | GenBank | Hs.289293 | fulllength | 157695 | NM_175075; hypothetical protein INM01 |
| 65 | AA872727 | Hs.48876.1 | GenBank | Hs.48876 | fulllength | 2222 | NM_004462; farnesyl-diphosphate farnesyltransferase 1 |
| 66 | BC003573.1 | g13097746 | GenBank | Hs.48876 | fulllength | 2222 | NM_004462; farnesyl-diphosphate farnesyltransferase 1 |
| 67 | NM_018361.1 | g8922941 | RefSeq | Hs.27842 | fulllength | 55326 | NM_018361; acid acyltransferase-epsilon |
| 68 | AA083514 | Hs.68301.0.A1 | GenBank | Hs.359201 | est | | |
| 69 | NM_001147.1 | g4557314 | RefSeq | Hs.115181 | fulllength | 285 | NM_001147; angiopoietin 2 |
| 70 | BG168896 | Hs.138381.1.A1 | GenBenk | Hs.356463 | fulllength | 2339 | NM_002027; farnesyltransferase, CAAX box, alpha |
| 71 | L00634.1 | g292030 | GenBank | Hs.356463 | fulllength | 2339 | NM_002027; farnesyltransferase, CAAX box, alpha |
| 72 | BG168896 | Hs.138381.1.A1 | GenBank | Hs.356463 | fulllength | 2339 | NM_002027; farnesyltransferase, CAAX box, alpha |
| 73 | W94952 | Hs.226318.1.A2 | GenBank | Hs.380963 | fulllength | 29883 | NM_013354: CCR4-NOT transcription complex, subunit 7 isoform 1 |
| | | | | | | | NM_054026; CCR4-NOT transcription complex, subunit 7 isoform 2 |
| 74 | NM_013354.2 | g10518495 | RefSeq | Hs.380963 | fulllength | 29883 | NM_013354: CCR4-NOT transcription complex, subunit 7 isoform 1 |
| | | | | | | | NM_054026: CCR4-NOT transcription complex, subunit 7 isoform 2 |
| 75 | NM_006197.1 | g5453855 | RefSeq | Hs.75737 | fulllength | 5108 | NM_006197; pericentriolar material 1 |
| 76 | AI205598 | Hs.75737.1.S1 | GenBank | Ns.75737 | fulllength | 5108 | NM_006197; pericentriolar material 1 |
| 77 | AI924817 | Hs.75737.2.S1 | GenBank | Hs.75737 | fulllength | 5108 | NM_006197; pericentriolar material 1 |
| 78 | AU145061 | Hs.287574.0.S1 | GenBank | Hs.287574 | | | |
| 79 | NM_001693.1 | g4502310 | RefSeq | Hs.1697 | fulllength | 526 | NM_001693; ATPase, H+ transporting, lysosomal 56/58 kD, V1 subunit |
| | | | | | | | B, isoform 2 |
| 80 | AI866426 | Hs.101617.0_RC | GenBank | Hs.101617 | fulllength | 137492 | NM_152415; hypothetical protein FLJ32642 |
| 81 | AW028100 | Hs.290855.0 | GenBank | Hs.101617 | fulllength | 137492 | NM_152415; hypothetical protein FLJ32642 |
| 82 | NM_014846.1 | p7661987 | RefSeq | Hs.8294 | fulllength | 9897 | NM_014846; KIAA0196 gene product |
| 83 | U42349.1 | p1353672 | GenBank | Hs.71119 | fulllength | 7991 | NM_006765; Putative prostate cancer tumor suppressor isoform a |
| | | | | | | | NM_178234: Putative prostate cancer tumor suppressor isoform b |
| 84 | NM_022749.1 | g12232410 | RefSeq | Hs.299148 | fulllength | 64760 | NM_022749: retinoic acid induced 16 |
| 85 | AI828929 | Hs.71215.0 | GenBank | Hs.71215 | fulllength | 9046 | NM_003974: docking protein 2, 56 kD |
| 86 | AK001289.1 | Ns.68090.1.S1 | GenBank | Hs.68090 | fulllength | 55909 | NM_018688; bridging integrator 3 |
| 87 | NM_005605.1 | g5031988 | RefSeq | Hs.75206 | fulllength | 5533 | NM_005605; protein phosphatase 3 (formerly 2B), catalytic subunit. |
| | | | | | | | gamma isoform (calcineurin A gamma) |
| 88 | AL519427 | Hs.296360.0.S1 | GenBank | Hs.375560 | fulllength | 286057 | NM_173686; hypothetical protein FLJ34715 |
| 89 | AF037261.1 | g3004947 | GenBank | Hs.33787 | fulllength | 10174 | NM_005775; vinexin bela (SH3-containing adaptor molecule-1) |
| 90 | S46622 | 4922761 | GenBank | Hs.75208 | fulllength | 5533 | NM_005605; protein phosphatase 3 (formerly 2B), catalytic subunit, |
| | | | | | | | gamma isoform (calcineurin A gamma) |
| 91 | NM_014479.1 | g7657318 | RefSeq | Ns.145296 | fulllength | 27299 | NM_014479; disintegrin protease |
| 92 | AK021850.1 | Hs.27410.2.S1 | GenBank | Hs.27410 | fulllength | 55893 | NM_018660; papillomavirus regulatory factor PRF-1 |
| 93 | BC001237.1 | g12654788 | GenBank | Hs.27410 | fulllength | 55893 | NM_018660; papillomavirus regulatory factor PRF-1 |
| 94 | AI949204 | Hs.267905.2_RC | GenBank | Hs.267905 | fulllength | 55140 | NM_018091; elongation protein 3 homolog |
| 95 | NM_015254.1 | g13194196 | RefSeq | Hs.15711 | fulllength | 23303 | NM_015254; kinesin family member 13B |
| 96 | BF196856 | Hs.135191.0.A1 | GenBank | Hs.438933 | fulllength | 157574 | NM_172366; F-box only protein 16 |
| 97 | NM_024567.1 | g13375737 | RefSeq | Hs.23590 | fulllength | 79618 | NM_024567; hypothetical protein FLJ21616 |
| 98 | AI638208 | Hs.223267.0.A1 | GenBank | Hs.201058 | est | | |
| 99 | BF055311 | Hs.302689.1.S1 | GenBank | Hs.211584 | fulllength | 4747 | NM_006158: neurofilament, light polypeptide 68 kDa |
| 100 | NM_006132.1 | g5902814 | RefSeq | Hs.1274 | fulllength | 649 | NM_001199; bone morphogenetic protein 1 isoform 1, precursor |
| | | | | | | | NM_006128; bone morphogenetic protein 1 isoform 2, precursor |
| | | | | | | | NM_006129; bone morphogenetic protein 1 isoform 3, precursor |
| | | | | | | | NM_006130; bone morphogenetic protein 1 isoform 6, precursor |
| | | | | | | | NM_006131; bone morphogenetic protein 1 isoform 5, precursor |
| | | | | | | | NM_006132; bone morphogenetic protein 1 isoform 4, precursor |
| 101 | AA679589 | Hs.308435.0.A1 | GenBank | Hs.308435 | est | | |
| 102 | NM_004156.1 | g4758951 | RefSeq | Hs.80350 | fulllength | 5516 | NM_004156; protein phosphatase 2 (formerly 2A), catalytic subunit, beta |
| | | | | | | | isoform |
| 103 | BC002671.1 | g12803670 | GenBank | Hs.2359 | fulllength | 1846 | NM_001394; dual specificity phosphatase 4 isoform 1 NM_057158; dual |
| | | | | | | | specificity phosphatase 4 isoform 2 |
| 104 | NM_006867.1 | g5803140 | RefSeq | Hs.80248 | fulllength | 11030 | NM_006867; RNA-binding protein with multiple splicing |
| 105 | AI147738 | Hs.132665.0.A1 | GenBank | Hs.326744 | fulllength | 84750 | NM_032664; fucosyltransferase 10 |
| 106 | AB044548.1 | g11527778 | GenBank | Hs.433317 | fulllength | 1978 | NM_004095; eukaryotic translation initiation factor 4E binding protein 1 |
| 107 | NM_023110.1 | g13186246 | RefSeq | Hs.748 | fulllength | 2260 | NM_000604; fibroblast growth factor receptor 1 isoform 1 precursor |
| | | | | | | | NM_015850; fibroblast growth factor receptor 1 isoform 2 precursor |
| | | | | | | | NM_023105; fibroblast growth factor receptor 1 isoforrn 3 precursor |
| | | | | | | | NM_023106; fibroblast growth factor receptor 1 isoform 4 precursor |
| | | | | | | | NM_023107; fibroblast growth factor receptor 1 isoform 5 precursor |
| | | | | | | | NM_023108; fibroblast growth factor receptor 1 isoform 6 precursor |
| | | | | | | | NM_023109; fibroblast growth factor receptor 1 isoform 7 precursor |
| | | | | | | | NM_023110; fibroblast growth factor receptor 1 isoform 8 precursor |
| | | | | | | | NM_023111; fibroblast growth factor receptor 1 isoform 9 precursor |
| 108 | AB018268.1 | Hs.26450.0_RC | GenBank | Hs.26450 | fulllength | 23259 | |
| 109 | AW292751 | Hs.28274.0 | GenBank | Hs.423737 | | | |
| 110 | AF353991.1 | g13625460 | GenBank | Hs.7471 | fulllength | 83877 | NM_031940; BBP-like protein 1 isoform b NM_078473; BBP-like protein |
| | | | | | | | 1 isoform a |
| 111 | AV711345 | Hs.10018.1.A1 | GenBank | Hs.10018 | fulllength | 114926 | NM_138436; hypothetical protein BC013035 |
| 112 | AI697751 | Hs.27721.0_RC | GenBank | Hs.27721 | fulllength | 54904 | NM_017778; WHSC1L1 protein isoform short NM_023034; WHSC1L1 |
| | | | | | | | protein isoform long |
| 113 | AA176833 | Hs.301959.0.A2 | GenBank | Hs.301959 | fulllength | 11212 | NM_007198; proline synthetase co-transcribed homolog |
| 114 | NM_002690.1 | g4505930 | RefSeq | Hs.180107 | fulllength | 5423 | NM_002690; polymerase (DNA directed), beta |
| 115 | AF255649.1 | g12005822 | GenBenk | Hs.27721 | fulllength | 54904 | NM_017778; WHSC1L1 protein isoform short NM_023034; WHSC1L1 |
| | | | | | | | protein isoform long |
| 116 | NM_020130.1 | g9910147 | RefSeq | Hs.283683 | fulllength | 56892 | NM_020130; chromosome 8 open reading frame 4 |
| 117 | NM_025070.1 | g13376612 | RefSeq | Hs.288057 | fulllength | 80140 | NM_025070; hypothetical protein FLJ22242 |
| 118 | X79990.1 | Hs.31551.0 | GenBank | Hs.31551 | fulllength | 862 | NM_004349; acute myelogenous leukemia 1 translocation 1 protein |
| | | | | | | | isoform MTG8a NM_175634; acute myelogenous leukemia 1 |
| | | | | | | | translocation 1 protein isoform MTG8b NM_175635; acute myelogenous |
| | | | | | | | leukemia 1 translocation 1 protein isoform MTG8c NM_175636; acute |
| | | | | | | | myelogenous leukemia 1 translocation 1 protein isoform MTG8c |
| 119 | AI217416 | Hs.90858.0.S1 | GenBank | Hs.90858 | | | |
| 120 | AI884858 | Hs.71119.1_RC | GenBank | Hs.71119 | fulllength | 7991 | NM_006765; Putative prostate cancer tumor suppressor isoform a |
| | | | | | | | NM_178234; Putative prostate cancer tumor suppressor isoform b |

TABLE 35
MDS_AML_Blasten

| # | affy id | HUGO name | p | q | r | Title | MapLocation | Sequence Type | Transcript ID | Sequence Derived From |
|---|---|---|---|---|---|---|---|---|---|---|
| 1 | 201061_s_at | STOM | 0.00e+00 | 0.00e+00 | −0.76 | stomatin | 9q34.1 | Exemplarsequence | Hs.160483.0 | M81635.1 |
| 2 | 211883_x_at | CEACAM1 | 0.00e+00 | 0.00e+00 | −0.75 | carcinoembryonic antigen-related cell | 19q13.2 | Exemplarsequence | Hs.50964.4 | M76742.1 |
| | | | | | | adhesion molecule 1 (biliary glycoprotein) | | | | |
| 3 | 202018_s_at | LTF | 0.00e+00 | 0.00e+00 | −0.74 | lactotransferrin | 3q21-q23 | Exemplarsequence | Hs.105938.0 | NM_002343.1 |
| 4 | 206676_at | CEACAM8 | 0.00e+00 | 0.00e+00 | −0.74 | carcinoembryonic antigen-related cell | 19q13.2 | Exemplarsequence | Hs.41.0 | M33326.1 |
| | | | | | | adhesion molecule 8 | | | | |
| 5 | 206177_s_at | ARG1 | 0.00e+00 | 0.00e+00 | −0.74 | arginasa, liver | 6q23 | Exemplarsequence | Hs.289057.0 | NM_000045.2 |
| 6 | 211657_at | CEACAM6 | 0.00e+00 | 0.00e+00 | −0.73 | carcinoembryonic antigen-related cell | 19q13.2 | Exemplarsequence | g189084 | M18728.1 |
| | | | | | | adhesion molecule 6 (non-specific cross | | | | |
| | | | | | | reacting antigen) | | | | |
| 7 | 209369_at | ANXA3 | 0.00e+00 | 0.00e+00 | −0.73 | annexin A3 | 4q13-q22 | Exemplarsequence | Hs.1378.0 | M63310.1 |
| 8 | 207802_at | SGP28 | 0.00e+00 | 0.00e+00 | −0.73 | specific granule protein (28 kDa) | 6p12.3 | Exemplarsequence | Hs.54431.0 | NM_006061.1 |
| 9 | 201060_x_at | STOM | 0.00e+00 | 0.00e+00 | −0.73 | stomatin | 9q34.1 | Consensussequence | Hs.160463.0 | AI537887 |
| 10 | 212531_at | LCN2 | 0.00e+00 | 0.00e+00 | −0.72 | lipocalin 2 (oncogene 24p3) | 9q34 | Consensussequence | Hs.204238.0 | NM_005564.1 |
| 11 | 209498_at | CEACAM1 | 0.00e+00 | 0.00e+00 | −0.72 | carcinoembryonic antigen-related cell | 19q13.2 | Consensussequence | Hs.50964.3 | X16354.1 |
| | | | | | | adhesion molecule 1 (biliary glycoprotein) | | | | |
| 12 | 204351_at | S100P | 0.00e+00 | 0.00e+00 | −0.72 | S100 calcium binding protein P | 4p16 | Exemplarsequence | Hs.2962.0 | NM_005980.1 |
| 13 | 232027_at | SYNE1 | 0.00e+00 | 0.00e+00 | −0.72 | spectrin repeat containing, nuclear | 6q25 | Consensussequence | Hs.241420.0 | AL049548 |
| | | | | | | envelope 1 | | | | |
| 14 | 226726_at | LOC129642 | 0.00e+00 | 0.00e+00 | −0.71 | hypothetical protein BC016005 | 2p25.2 | Consensussequence | Hs.15641.0 | W63676 |
| 15 | 203757_s_at | CEACAM6 | 0.00e+00 | 0.00e+00 | −0.71 | carcinoembryonic antigen-related cell | 19q13.2 | Exemplarsequence | Hs.73848.0 | BC005008.1 |
| | | | | | | adhasion molecule 6 (non-specific cross | | | | |
| | | | | | | reacting antigen) | | | | |
| 16 | 203936_s_at | MMP9 | 0.00e+00 | 0.00e+00 | −0.71 | matrix metalloproteinase 9 (gelatinase B, | 20q11.2-q13.1 | Exemplarsequence | Hs.151738.0 | NM_004994.1 |
| | | | | | | 92 kDa gelatinase, 92 kDa type IV | | | | |
| | | | | | | collagenase) | | | | |
| 17 | 210244_at | CAMP | 0.00e+00 | 0.00e+00 | −0.71 | cathelicidin antimicrobial peptide | 3p21.3 | Exemplarsequence | Hs.51120.0 | U19970.1 |
| 18 | 208650_s_at | CD24 | 0.00e+00 | 0.00e+00 | −0.71 | CD24 antigen (small cell lung carcinoma | 6q21 | Consensussequence | Hs.286124.1 | BG327863 |
| | | | | | | cluster 4 antigen) | | | | |
| 19 | 206515_at | CYP4F3 | 0.00e+00 | 0.00e+00 | −0.71 | cytochrome P450, family 4, subfamily F, | 19p13.2 | Exemplarsequence | Hs.106242.0 | NM_000896.1 |
| | | | | | | polypeptide 3 | | | | |
| 20 | 213836_s_at | FLJ10055 | 0.00e+00 | 0.00e+00 | −0.71 | hypothetical protein FLJ10055 | 17q24.3 | Consensussequence | Hs.279937.1 | AW052084 |
| 21 | 266_s_at | CD24 | 0.00e+00 | 0.00e+00 | −0.70 | CD24 antigen (small cell lung carcinoma | 6q21 | Exemplarsequence | L33930 | L33930 |
| | | | | | | cluster 4 antigen) | | | | |
| 22 | 209771_x_at | CD24 | 0.00e+00 | 0.00e+00 | −0.70 | CD24 antigen (small cell lung carcinoma | 6q21 | Consensussequence | Hs.286124.0 | AA761181 |
| | | | | | | cluster 4 antigen) | | | | |
| 23 | 211889_x_at | CEACAM1 | 0.00e+00 | 0.00e+00 | −0.70 | carcinoembryonic antigen-related cell | 19q13.2 | Exemplarsequence | Hs.50964.5 | D12502.1 |
| | | | | | | adhesion molecule 1 (biliary glycoprotein) | | | | |
| 24 | 206674_at | FLT3 | 0.00e+00 | 0.00e+00 | 0.70 | fms-related tyrosine kinase 3 | 13q12 | Exemplarsequence | Hs.385.0 | NM_004119.1 |
| 25 | 207384_at | PGLYRP | 0.00e+00 | 0.00e+00 | −0.70 | peptidoglycan recognition protein | 19q13.2-q13.3 | Exemplarsequence | Hs.137583.0 | NM_005091.1 |
| 26 | 226188_at | HSPC159 | 0.00e+00 | 0.00e+00 | −0.69 | HSPC159 protein | 2p13.3 | Consensussequence | Hs.7149.0 | AK025603.1 |
| 27 | 216379_x_at | KIAA1919 | 0.00e+00 | 0.00e+00 | −0.69 | KIAA1919 protein | 6q22 | Consensussequence | Hs.332045.0 | AK000168.1 |
| 28 | 210638_s_at | FBXO9 | 0.00e+00 | 0.00e+00 | −0.69 | F-box only protein 9 | 6p12.3-p11.2 | Exemplarsequence | Hs.11050.1 | AF176704.1 |
| 29 | 207072_at | IL18RAP | 0.00e+00 | 0.00e+00 | −0.69 | interleukin 18 receptor accessory protein | 2p24.3-p24.1 | Exemplarsequence | Hs.158315.0 | NM_003853.1 |
| 30 | 205389_s_at | ANK1 | 0.00e+00 | 0.00e+00 | −0.69 | ankyrin 1, erythrocytic | 8p11.1 | Consensussequence | Hs.183805.0 | AI659683 |
| 31 | 202197_at | MTMR3 | 0.00e+00 | 0.00e+00 | −0.69 | myotubularin related protein 3 | 22q12.2 | Exemplarsequence | Hs.63302.0 | NM_021090.1 |
| 32 | 207674_at | FCAR | 0.00e+00 | 0.00e+00 | −0.69 | Fc fragment of IgA, receptor for | 19q13.2-q13.4 | Exemplarsequence | Hs.193122.0 | NM_002000.1 |
| 33 | 203765_at | GCA | 0.00e+00 | 0.00e+00 | −0.68 | grancalcin, EF-hand calcium binding | 2q24.3 | Exemplarsequence | Hs.79381.0 | NM_012198.1 |
| | | | | | | protein | | | | |
| 34 | 223423_at | GPCR1 | 0.00e+00 | 0.00e+00 | −0.68 | putative G protein-coupled receptor | 3q26.2-q27 | Exemplarsequence | Hs.97101.0 | BC000181.2 |
| 35 | 207992_s_at | AMPD3 | 0.00e+00 | 0.00e+00 | −0.68 | adenosine monophosphate deaminase | 11p15 | Exemplarsequence | Hs.83918.0 | NM_000480.1 |
| | | | | | | (isoform E) | | | | |
| 36 | 224828_at | KIAA1673 | 0.00e+00 | 0.00e+00 | −0.68 | KIAA1673 | 5q21 | Consensussequence | Hs.301444.0 | AA772278 |
| 37 | 208651_x_at | CD24 | 0.00e+00 | 0.00e+00 | −0.68 | CD24 antigen (small cell lung carcinoma | 6q21 | Exemplarsequence | Hs.286124.1 | M58664.1 |
| | | | | | | cluster 4 antigen) | | | | |
| 38 | 207459_x_at | GYPB | 0.00e+00 | 0.00e+00 | −0.68 | glycophorin B (includes Ss blood group) | 4q28-q31 | Exemplarsequence | Hs.250653.0 | NM_002100.2 |
| 39 | 221765_at | UGCG | 0.00e+00 | 0.00e+00 | −0.68 | UDP-glucose ceramide | 9q31 | Consensussequence | Hs.23703.0 | BF970427 |
| | | | | | | glucosyltransferase | | | | |
| 40 | 212987_at | FBXO9 | 0.00e+00 | 0.00e+00 | −0.68 | F-box only protein 9 | 6p12.3-p11.2 | Consensussequence | Hs.11050.3 | AL137520.1 |
| 41 | 205442_at | KIAA0626 | 0.00e+00 | 0.00e+00 | −0.68 | KIAA0626 gene product | 4q32.3 | Exemplarsequence | Hs.178121.0 | NM_021647.1 |
| 42 | 220404_at | | 0.00e+00 | 0.00e+00 | −0.68 | ESTs | | Exemplarsequence | Hs.163833.0 | NM_014076.1 |
| 43 | 207087_x_at | ANK1 | 0.00e+00 | 0.00e+00 | −0.67 | ankyrin 1, erythrocytic | 8p11.1 | Exemplarsequence | Hs.183805.1 | NM_020478.1 |
| 44 | 228361_at | | 0.00e+00 | 0.00e+00 | −0.67 | Homo sapiens, Similar to hypothetical | | Consensussequence | Hs.49047.0 | AL561296 |
| | | | | | | protein PRO1722, clone MGC: 15692 | | | | |
| | | | | | | IMAGE: 3351479, mRNA, complete cds | | | | |
| 45 | 224967_at | UGCG | 0.00e+00 | 0.00e+00 | −0.67 | UDP-glucose ceramide | 9q31 | Consensussequence | Hs.23703.0 | W72338 |
| | | | | | | glucosyltransferase | | | | |
| 46 | 221778_at | KIAA1718 | 0.00e+00 | 0.00e+00 | −0.67 | KIAA1718 protein | 7q33-q35 | Consensussequence | Hs.222707.0 | BE217882 |
| 47 | 208353_x_at | ANK1 | 0.00e+00 | 0.00e+00 | −0.67 | ankyrin 1, erythrocytic | 8p11.1 | Exemplarsequence | Hs.183805.6 | NM_020480.1 |
| 48 | 205391_x_at | ANK1 | 0.00e+00 | 0.00e+00 | −0.67 | ankyrin 1, erythrocytic | 8p11.1 | Exemplarsequence | Hs.183805.0 | M28880.1 |
| 49 | 209760_at | KIAA0922 | 0.00e+00 | 0.00e+00 | −0.67 | KIAA0922 protein | 4q31.3 | Exemplarsequence | Hs.37892.0 | AL136932.1 |
| 50 | 216833_x_at | GYPB | 0.00e+00 | 0.00e+00 | −0.67 | glycophorin B (includes Ss blood group) | 4q28-q31 | Consensussequence | Hs.307185.0 | U05255.1 |
| 51 | 207269_at | DEFA4 | 0.00e+00 | 0.00e+00 | −0.67 | defensin, alpha 4, corticostatin | 8p23 | Exemplarsequence | Hs.2582.0 | NM_001925.1 |
| 52 | 231688_at | | 0.00e+00 | 0.00e+00 | −0.67 | ESTs | | Consensussequence | Hs.255877.0 | AW337833 |
| 53 | 205557_at | BPI | 0.00e+00 | 0.00e+00 | −0.67 | bactericidal/permeability-increasing protein | 20q11.23-q12 | Exemplarsequence | Hs.89535.0 | NM_001725.1 |
| 54 | 209772_s_at | CD24 | 0.00e+00 | 0.00e+00 | −0.66 | CD24 antigen (small cell lung carcinoma | 6q21 | Exemplarsequence | Hs.286124.0 | X69397.1 |
| | | | | | | cluster 4 antigen) | | | | |
| 55 | 204881_s_at | UGCG | 0.00e+00 | 0.00e+00 | −0.66 | UDP-glucose ceramide | 9q31 | Exemplarsequence | Hs.152601.0 | NM_003358.1 |
| | | | | | | glucosyltransferase | | | | |
| 56 | 239205_s_at | CR1L | 0.00e+00 | 0.00e+00 | −0.66 | complement component (3b/4b) receptor 1- | 1q32.1 | Consensussequence | Hs.89688.0 | BE552138 |
| | | | | | | like | | | | |
| 57 | 221748_s_at | | 0.00e+00 | 0.00e+00 | −0.66 | Homo sapiens cDNA FLJ32766 fis, clone | | Consensussequence | Hs.9973.1 | AL046979 |
| | | | | | | TESTI2001862. | | | | |
| 58 | 206302_s_at | NUDT4 | 0.00e+00 | 0.00e+00 | −0.66 | nudix (nucleoside diphosphate linked | | Exemplarsequence | Hs.92381.0 | NM_019094.1 |
| | | | | | | moiety X)-type motif 4 | | | | |
| 59 | 206283_s_at | TAL1 | 0.00e+00 | 0.00e+00 | −0.66 | T-cell acute lymphocytic leukemia 1 | 1p32 | Exemplarsequence | Hs.73828.0 | NM_003189.1 |
| 60 | 200998_s_at | CKAP4 | 0.00e+00 | 0.00e+00 | −0.66 | cytoskeleton-associated protein 4 | 12q24.11 | Consensussequence | Hs.74368.0 | AW029619 |
| 61 | 37986_at | EPOR | 0.00e+00 | 0.00e+00 | −0.66 | erythropoietin receptor | 19p13.3-p13.2 | Consensussequence | 4876922_rc | M60459 |
| 62 | 211207_s_at | FACL6 | 0.00e+00 | 0.00e+00 | −0.66 | fatty-acid-Coenzyme A ligase, long-chain 6 | 5q31 | Exemplarsequence | Hs.14945.0 | AF129166.1 |
| 63 | 211820_x_at | GYPA | 0.00e+00 | 0.00e+00 | −0.66 | glycophorin A (includes MN blood group) | 4q28.2-q31.1 | Exemplarsequence | Hs.108694.2 | U00179.1 |
| 64 | 218853_s_at | DJ473B4 | 0.00e+00 | 0.00e+00 | −0.66 | hypothetical protein dJ473B4 | Xq26.3 | Exemplarsequence | Hs.57549.0 | NM_019556.1 |
| 65 | 209396_s_at | CHI3L1 | 0.00e+00 | 0.00e+00 | −0.66 | chitinase 3-like 1 (cartilage glycoprotein- | 1q32.1 | Exemplarsequence | Hs.75184.0 | M80927.1 |
| | | | | | | 39) | | | | |
| 66 | 205513_at | TCN1 | 0.00e+00 | 0.00e+00 | −0.66 | transcobalamin I (vitamin B12 binding | 11q11-q12 | Exemplarsequence | Hs.2012.0 | NM_001062.1 |
| | | | | | | protein, R binder family) | | | | |
| 67 | 205592_at | SLC4A1 | 0.00e+00 | 0.00e+00 | −0.66 | solute carrier family 4, anion exchanger, | 17q21-q22 | Consensussequence | Hs.185923.0 | X77737.1 |
| | | | | | | member 1 (erythrocyte membrane protein | | | | |
| | | | | | | band 3, Diego blood group) | | | | |
| 68 | 236081_at | | 0.00e+00 | 0.00e+00 | −0.66 | ESTs | | Consensussequence | Hs.21374.0 | AI929792 |
| 69 | 223894_s_at | FTS | 0.00e+00 | 0.00e+00 | −0.66 | fused toes homolog (mouse) | 16q12.1 | Exemplarsequence | Hs.288929.1 | BC001134.1 |
| 70 | 229889_at | MGC40157 | 0.00e+00 | 0.00e+00 | −0.65 | hypothetical protein MGC40157 | 17p11.2 | Consensussequence | Hs.295362.1 | AW137009 |
| 71 | 209212_s_at | KLF5 | 0.00e+00 | 0.00e+00 | −0.65 | Kruppel-like factor 5 (intestinal) | 13q21.32 | Exemplarsequence | Hs.84728.0 | AB030824.1 |
| 72 | 239206_at | CR1L | 0.00e+00 | 0.00e+00 | −0.65 | complement component (3b/4b) receptor 1- | 1q32.1 | Consensussequence | Hs.89688.0 | BE552138 |
| | | | | | | like | | | | |
| 73 | 210004_at | OLR1 | 0.00e+00 | 0.00e+00 | −0.65 | oxidised low density lipoprotein (lectin-like) | 12p13.2-p12.3 | Exemplarsequence | Hs.77729.0 | AF035776.1 |
| | | | | | | receptor 1 | | | | |
| 74 | 208352_x_at | ANK1 | 0.00e+00 | 0.00e+00 | −0.65 | ankyrin 1, erythrocytic | 8p11.1 | Exemplarsequence | Hs.183805.5 | NM_020479.1 |
| 75 | 211821_x_at | GYPA | 0.00e+00 | 0.00e+00 | −0.65 | glycophorin A (includes MN blood group) | 4q28.2-q31.1 | Exemplarsequence | Hs.108694.1 | U00178.1 |
| 76 | 214407_x_at | GYPB | 0.00e+00 | 0.00e+00 | −0.65 | glycophorin B (includes Ss blood group) | 4q28-q31 | Consensussequence | Hs.250653.1 | AI240545 |
| 77 | 214523_at | CEBPE | 0.00e+00 | 0.00e+00 | −0.65 | CCAAT/enhancer binding protein (C/EBP), | 14q11.2 | Consensussequence | Hs.158323.0 | NM_001805.1 |
| | | | | | | epsilon | | | | |
| 78 | 226179_at | | 0.00e+00 | 0.00e+00 | −0.65 | Homo sapiens, clone IMAGE: 5294823, | | Consensussequence | Hs.34549.0 | N63920 |
| | | | | | | mRNA | | | | |
| 79 | 203827_at | FLJ10055 | 0.00e+00 | 0.00e+00 | −0.65 | hypothetical protein FLJ10055 | 17q24.3 | Exemplarsequence | Hs.9398.0 | NM_017983.1 |
| 80 | 208942_s_at | TLOC1 | 0.00e+00 | 0.00e+00 | −0.65 | translocation protein 1 | 3q26.2-q27 | Consensussequence | Hs.8146.0 | BE866511 |
| 81 | 203845_at | PCAF | 0.00e+00 | 0.00e+00 | −0.65 | p300/CBP-associated factor | 3p24 | Consensussequence | Hs.199061.0 | AV727449 |
| 82 | 209395_at | CHI3L1 | 0.00e+00 | 0.00e+00 | −0.65 | chitinase 3-like 1 (cartilage glycoprotein- | 1q32.1 | Exemplarsequence | Hs.75184.0 | M80927.1 |
| | | | | | | 39) | | | | |
| 83 | 205837_s_at | GYPA | 0.00e+00 | 0.00e+00 | −0.65 | glycophorin A (includes MN blood group) | 4q28.2-q31.1 | Exemplarsequence | Hs.108694.0 | BC005319.1 |
| 84 | 216925_s_at | TAL1 | 0.00e+00 | 0.00e+00 | −0.65 | T-cell acute lymphocytic leukemia 1 | 1p32 | Consensussequence | Hs.73828.1 | X51990.1 |
| 85 | 207329_at | MMP8 | 0.00e+00 | 0.00e+00 | −0.65 | matrix metalloproteinase 8 (neutrophil | 11q22.3 | Exemplarsequence | Hs.73862.0 | NM_002424.1 |
| | | | | | | collagenase) | | | | |
| 86 | 228376_at | a1/3GTP | 0.00e+00 | 0.00e+00 | −0.65 | alpha-1,3-galactosyltransferase | 9q33.3 | Consensussequence | Hs.97469.0 | AI972498 |
| | | | | | | pseudogene | | | | |
| 87 | 234980_at | FLJ31842 | 0.00e+00 | 0.00e+00 | −0.65 | hypothetical protein FLJ31842 | 1p21.3 | Consensussequence | Hs.84522.0 | AI004375 |
| 88 | 221485_at | B4GALT5 | 0.00e+00 | 0.00e+00 | −0.65 | UDP-Gal:betaGlcNAc beta 1,4- | 20q13.1-q13.2 | Consensussequence | Hs.107526.0 | NM_004776.1 |
| | | | | | | galactosyltransferase, polypeptide 5 | | | | |
| 89 | 229610_at | FLJ40629 | 0.00e+00 | 0.00e+00 | −0.65 | hypothetical protein FLJ40629 | 2q13 | Consensussequence | Hs.99807.0 | AW088063 |
| 90 | 220807_at | HBQ1 | 0.00e+00 | 0.00e+00 | −0.65 | hemoglobin, theta 1 | 16p13.3 | Exemplarsequence | Hs.247921.0 | NM_005331.2 |
| 91 | 210854_x_at | SLC6A8 | 0.00e+00 | 0.00e+00 | −0.65 | solute carrier family 6 (neurotransmitter | Xq28 | Exemplarsequence | Hs.187958.1 | U17986.1 |
| | | | | | | transporter, creatine), member 8 | | | | |
| 92 | 210746_s_at | EPB42 | 0.00e+00 | 0.00e+00 | −0.65 | erythrocyte membrane protein band 4.2 | 15q15-q21 | Exemplarsequence | Hs.733.1 | M30646.1 |
| 93 | 216054_x_at | MYL4 | 0.00e+00 | 0.00e+00 | −0.65 | myosin, light polypeptide 4, alkali; atrial, | 17q21-qter | Consensussequence | Hs.298161.3 | X58851 |
| | | | | | | embryonic | | | | |
| 94 | 210395_x_at | MYL4 | 0.00e+00 | 0.00e+00 | −0.64 | myosin, light polypeptide 4, alkali; atrial, | 17q21-qter | Consensussequence | Hs.298161.0 | AF116676.1 |
| | | | | | | embryonic | | | | |
| 95 | 201362_at | NS1-BP | 0.00e+00 | 0.00e+00 | −0.64 | NS1-binding protein | 1q25.1-q31.1 | Exemplarsequence | Hs.197298.0 | AF205218.1 |
| 96 | 204467_s_at | SNCA | 0.00e+00 | 0.00e+00 | −0.64 | synuclein, alpha (non A4 component of | 4q21 | Eaemplarsequence | Hs.76930.1 | NM_000345.2 |
| | | | | | | amyloid precursor) | | | | |
| 97 | 206522_at | MGAM | 0.00e+00 | 0.00e+00 | −0.64 | maltase-glucoamylase (alpha-glucosidase) | 7q34 | Exemplarsequence | Hs.122785.0 | NM_004668.1 |
| 98 | 213805_at | CGI-58 | 0.00e+00 | 0.00e+00 | −0.64 | CGI-58 protein | 3p25.3-p24.3 | Consensussequence | Hs.184019.1 | AI692428 |
| 99 | 227309_at | | 0.00e+00 | 0.00e+00 | −0.64 | ESTs | | Consensussequence | Hs.8688.0 | AI982535 |
| ## | 219672_at | ERAF | 0.00e+00 | 0.00e+00 | −0.64 | erythroid associated factor | 16p11.2 | Exemplarsequence | Hs.274309.0 | NM_016633.1 |

| # | Sequence ID | Sequence Source | Unigene_Accession | Cluster_Type | LocusLink | Full_Length_Reference_Seq |
|---|---|---|---|---|---|---|
| 1 | g181183 | GenBank | Hs.160483 | fulllength | 2040 | NM_004099; stomatin |
| 2 | g179480 | GenBank | Hs.50964 | fulllength | 634 | NM_001712; carcinoembryonic antigen-related cell adhesion molecule 1 (biliary glycoprotein) |
| 3 | g4505042 | RefSeq | Hs.105938 | fulllength | 4057 | NM_002343; lactotransferrin |
| 4 | g189101 | GenBank | Hs.41 | fulllength | 1088 | NM_001816; carcinoembryonic antigen-related cell adhesion molecule 8 |
| 5 | g10947138 | RefSeq | Hs.332405 | fulllength | 383 | NM_000045; arginase, type I |
| 6 | g189084 | GenBank | Hs.73848 | fulllength | 4680 | NM_002483; carcinoembryonic antigen-related cell adhesion molecule 6 (non-specific cross reacting antigen) |
| 7 | g178696 | GenBank | Hs.1378 | fulllength | 306 | NM_005139; annexin A3 |
| 8 | g5174674 | RefSeq | Hs.54431 | fulllength | 10321 | NM_006061; specific granule protein (28 kDa) |
| 9 | Hs.160483.0 | GenBank | Hs.160483 | fulllength | 2040 | NM_004099; stomatin |
| 10 | Hs.204238.0.S1 | GenBank | Hs.204238 | fulllength | 3934 | NM_005564; lipocalin 2 (oncogene 24p3) |
| 11 | Hs.50964.3 | GenBank | Hs.50964 | fulllength | 634 | NM_001712; carcinoembryonic antigen-related cell adhesion molecule 1 (biliary glycoprotein) |
| 12 | g5174662 | RefSeq | Hs.2962 | fulllength | 6286 | NM_005980; S100 calcium binding protein P |
| 13 | Hs.241420.0.S1 | GenBank | Hs.192102 | fulllength | 23345 | NM_015293; nesprin 1 isoform beta NM_033071; nesprin 1 isoform longest NM_133650; nesprin 1 isoform alpha |
| 14 | Hs.15641.0.A1 | GenBank | Hs.356547 | fulllength | 129642 | NM_138799; hypothetical protein BC016005 |
| 15 | g13477106 | GenBank | Hs.73848 | fulllength | 4680 | NM_002483; carcinoembryonic antigen-related cell adhesion molecule 6 (non-specific cross reacting antigen) |
| 16 | g4826835 | RefSeq | Hs.151738 | fulllength | 4318 | NM_004994; matrix metalloproteinase 9 preproprotein |
| 17 | g643476 | GenBank | Hs.51120 | fulllength | 820 | NM_004345; cathelicidin antimicrobial peptide |
| 18 | Hs.286124.1_RC | GenBank | Hs.375108 | fulllength | 934 | NM_013230; CD24 antigen (small cell lung carcinoma cluster 4 antigen) |
| 19 | 24503240 | RefSeq | Hs.106242 | fulllength | 4051 | NM_000896; cytochrome P450, family 4, subfamily F, polypeptide 3 |
| 20 | Hs.279937.1_RC | GenBank | Hs.9398 | fulllength | 55062 | NM_014960; KIAA1001 protein NM_017983; hypothetical protein FLJ10055 |
| 21 | L33930 | GenBank | Hs.375108 | fulllength | 934 | NM_013230; CD24 antigen (small cell lung carcinoma cluster 4 antigen) |
| 22 | Hs.286124.0_RC | GenBank | Hs.375108 | fulllength | 934 | NM_013230; CD24 antigen (small cell lung carcinoma cluster 4 antigen) |
| 23 | g219494 | GenBank | Hs.50964 | fulllength | 634 | NM_001712; carcinoembryonic antigen-related cell adhesion molecule 1 (biliary glycoprotein) |
| 24 | g4758395 | RefSeq | Hs.385 | fulllength | 2322 | NM_004119; fms-related tyrosine kinase 3 |
| 25 | g4827035 | RefSeq | Hs.137583 | fulllength | 8993 | NM_005091; peptidoglycan recognition protein |
| 26 | Hs.7149.0 | GenBank | Hs.372208 | fulllength | 29094 | NM_014181; HSPC159 protein |
| 27 | Hs.332045.0_RC | GenBank | Hs.400572 | fulllength | 91749 | NM_153369; KIAA1919 protein |
| 28 | g6103646 | GenBank | Hs.11050 | fulllength | 26268 | NM_012347; F-box only protein 9 isoform 1 NM_033480; F-box only protein 9 isoform 2 |
| | | | | | | NM_033481; F-box only protein 9 isoform 3 |
| 29 | g4504656 | RefSeq | Hs.158315 | fulllength | 8807 | NM_003853; Interleukin 18 receptor accessory protein precursor |
| 30 | Hs.183805.0.A2 | GenBank | Hs.183805 | fulllength | 286 | NM_000037; ankyrin 1 isoform 3 NM_020475; ankyrin 1 isoform 4 |
| | | | | | | NM_020476; ankyrin 1 isoform 1 NM_020477; ankyrin 1 isoform 2 |
| | | | | | | NM_020478; ankyrin 1 isoform 5 NM_020479; ankyrin 1 isoform 6 |
| | | | | | | NM_020480; ankyrin 1 isoform 7 NM_020481; ankyrin 1 isoform 8 |
| 31 | g10835108 | RefSeq | Hs.63302 | fulllength | 8897 | NM_021090; myotubularin-related protein 3 isoform c NM_153050; |
| | | | | | | myotubularin-related protein 3 isoform a NM_153051; myolubularin-related protein 3 isoform b |
| 32 | g4503672 | RefSeq | Hs.193122 | fulllength | 2204 | NM_002000; Fc alpha receptor isoform a precursor NM_133269; Fc alpha |
| | | | | | | receptor isoform b precursor NM_133271; Fc alpha receptor isoform c |
| | | | | | | precursor NM_133272; Fc alpha receptor isoform d NM_133273; Fc alpha |
| | | | | | | receptor isoform e NM_133274; Fc alpha receptor isoform f NM_133277; |
| | | | | | | Fc alpha receptor isoform g NM_133278; Fc alpha receptor isoform h |
| | | | | | | NM_133279; Fc alpha receptor isoform i precursor NM_133280; Fc alpha |
| | | | | | | receptor isoform j |
| 33 | g6912387 | RefSeq | Hs.79381 | fulllength | 25801 | NM_012198; grancalcin, EF-hand calcium binding protein |
| 34 | g13111815 | GenBank | Hs.97101 | fulllength | 26996 | NM_014373; putative G protein-coupled receptor |
| 35 | g4502078 | RefSeq | Hs.83918 | fulllength | 272 | NM_000480; adenosine monophosphate deaminase (isoform E) |
| 36 | Hs.301444.0.A1 | GenBank | Hs.301444 | fulllength | 80315 | |
| 37 | g180167 | GenBank | Hs.375108 | fulllength | 934 | NM_013230; CD24 antigen (small cell lung carcinoma cluster 4 antigen) |
| 38 | g8051603 | RefSeq | Hs.372513 | fulllength | 2994 | NM_002100; glycophorin B precursor |
| 39 | Hs.23703.0_RC | GenBank | Hs.432605 | fulllength | 7357 | NM_003358; ceramide glucosyltransferase |
| 40 | Hs.11050.3_RC | GenBank | Hs.11050 | fulllength | 26268 | NM_012347; F-box only protein 9 isoform 1 NM_033480; F-box only |
| | | | | | | protein 9 isoform 2 NM_033481; F-box only protein 9 isoform 3 |
| 41 | g11067364 | RefSeq | Hs.178121 | fulllength | 9848 | NM_021647; KIAA0626 gene product |
| 42 | g7662571 | RefSeq | Hs.383403 | est | | |
| 43 | g10947043 | RefSeq | Hs.183805 | fulllength | 286 | NM_000037; ankyrin 1 isoform 3 NM_020475; ankyrin 1 isoform 4 |
| | | | | | | NM_020476; ankyrin 1 isoform 1 NM_020477; ankyrin 1 isoform 2 |
| | | | | | | NM_020478; ankyrin 1 isoform 5 NM_020479; ankyrin 1 isoform 6 |
| | | | | | | NM_020480; ankyrin 1 isoform 7 NM_020481; ankyrin 1 isoform 8 |
| 44 | Hs.49047.0.A1 | GenBank | Hs.231444 | fulllength | | |
| 45 | Hs.23703.0.A2 | GenBank | Hs.432605 | fulllength | 7357 | NM_003358; ceramide glucosyltransferase |
| 46 | Hs.222707.0.A2 | GenBank | Hs.222707 | | 80853 | |
| 47 | g10947047 | RefSeq | Hs.183805 | fulllength | 286 | NM_000037; ankyrin 1 isoform 3 NM_020475; ankyrin 1 isoform 4 |
| | | | | | | NM_020476; ankyrin 1 isoform 1 NM_020477; ankyrin 1 isoform 2 |
| | | | | | | NM_020478; ankyrin 1 isoform 5 NM_020479; ankyrin 1 isoform 6 |
| | | | | | | NM_020480; ankyrin 1 isoform 7 NM_020481; ankyrin 1 isoform 8 |
| 48 | g178645 | GenBank | Hs.183805 | fulllength | 286 | NM_000037; ankyrin 1 isoform 3 NM_020475; ankyrin 1 isoform 4 |
| | | | | | | NM_020476; ankyrin 1 isoform 1 NM_020477; ankyrin 1 isoform 2 |
| | | | | | | NM_020478; ankyrin 1 isoform 5 NM_020479; ankyrin 1 isoform 6 |
| | | | | | | NM_020480; ankyrin 1 isoform 7 NM_020481; ankyrin 1 isoform 8 |
| 49 | g12053358 | GenBank | Hs.37892 | fulllength | 23240 | NM_015196; KIAA0922 protein |
| 50 | Hs.307185.0 | GenBank | Hs.372513 | fulllength | 2994 | NM_002100; glycophorin B precursor |
| 51 | g4503302 | RefSeq | Hs.2582 | fulllength | 1669 | NM_001925; defensin, alpha 4, preproprotein |
| 52 | Hs.255877.0.A1 | GenBank | Hs.255877 | est | | |
| 53 | g4502446 | RefSeq | Hs.89535 | fulllength | 671 | NM_001725; bactericidal/permeability-increasing protein precursor |
| 54 | g396167 | GenBank | Hs.375108 | fulllength | 934 | NM_013230; CD24 antigen (small cell lung carcinoma cluster 4 antigen) |
| 55 | g4507810 | RefSeq | Hs.432605 | fulllength | 7357 | NM_003358; ceramide glucosyltransferase |
| 56 | Hs.89688.0_RC | GenBank | Hs.89688 | | 1379 | |
| 57 | Hs.9973.1.A1 | GenBank | Hs.9973 | | | |
| 58 | g10800135 | RefSeq | Hs.355399 | fulllength | 11163 | NM_019094; nudix (nucleoside diphosphate linked moiety X)-type motif 4 |
| 59 | g4507362 | RefSeq | Hs.73828 | fulllength | 6886 | NM_003189; T-cell acute lymphocytic leukemia 1 |
| 60 | Hs.74368.0.A1 | GenBank | Hs.74368 | fulllength | 10970 | NM_006825; transmembrane protein (63 kD), endoplasmic reticulum/Golgi |
| | | | | | | interm |
| 61 | 4876922_rc | GenBank | Hs.127826 | fulllength | 2057 | NM_000121; erythropoietin receptor precursor |
| 62 | g5702201 | GenBank | Hs.14945 | fulllength | 23305 | NM_015256; fatty-acid-Coenzyme A ligase, long-chain 6 |
| 63 | g392432 | GenBank | Hs.108694 | fulllength | 2993 | NM_002099; glycophorin A precursor |
| 64 | g9506542 | RefSeq | Hs.57549 | fulllength | 56180 | NM_019556; hypothetical protein dJ473B4 |
| 65 | g348911 | GenBank | Hs.75184 | fulllength | 1116 | NM_001276; chitinase 3-like 1 |
| 66 | g4507406 | RefSeq | Hs.2012 | fulllength | 6947 | NM_001062; transcobalamin I precursor |
| 67 | Hs.185923.0 | GenBank | Hs.185923 | fulllength | 6521 | NM_000342; solute carrier family 4, anion exchanger, member 1 |
| | | | | | | (erythrocyte membrane protein band 3, Diego blood group) |
| 68 | Hs.21374.0_RC | GenBank | Hs.21374 | est | | |
| 69 | g12654596 | GenBank | Hs.288929 | fulllength | 64400 | NM_022476; fused toes homolog |
| 70 | Hs.295362.1.S1 | GenBank | Hs.295362 | fulllength | 125144 | NM_152350; hypothetical protein MGC40157 |
| 71 | g8272417 | GenBank | Hs.84728 | fulllength | 688 | NM_001730; Kruppel-like factor 5 |
| 72 | Hs.89688.0_RC | GenBank | Hs.89688 | | 1379 | |
| 73 | g3941299 | GenBank | Hs.77729 | fulllength | 4973 | NM_002543; oxidised low density lipoprotein (lectin-like) receptor 1 |
| 74 | g10947045 | RefSeq | Hs.183805 | fulllength | 286 | NM_000037; ankyrin 1 isoform 3 NM_020475; ankyrin 1 isoform 4 |
| | | | | | | NM_020476; ankyrin 1 isoform 1 NM_020477; ankyrin 1 isoform 2 |
| | | | | | | NM_020478; ankyrin 1 isoform 5 NM_020479; ankyrin 1 isoform 6 |
| | | | | | | NM_020480; ankyrin 1 isoform 7 NM_020481; ankyrin 1 isoform 8 |
| 75 | g392430 | GenBank | Hs.108694 | fulllength | 2993 | NM_002099; glycophorin A precursor |
| 76 | Hs.250653.1 | GenBank | Hs.372513 | fulllength | 2994 | NM_002100; glycophorin B precursor |
| 77 | Hs.158323.0 | GenBank | Hs.158323 | fulllength | 1053 | NM_001605; CCAAT/enhancer binding protein epsilon |
| 78 | Hs.34549.0.A1 | GenBank | Hs.34549 | | | |
| 79 | g8922207 | RefSeq | Hs.9398 | fulllength | 55062 | NM_017983; hypothetical protein FLJ10055 |
| 80 | Hs.8146.0_RC | GenBank | Hs.8146 | fulllength | 7095 | NM_003262; translocation protein 1 |
| 81 | Hs.199061.0.A2 | GenBank | Hs.199061 | fulllength | 8850 | NM_003884; p300/CBP-associated factor |
| 82 | g348911 | GenBank | Hs.75184 | fulllength | 1116 | NM_001276; chitinase 3-like 1 |
| 83 | g13529076 | GenBank | Hs.108694 | fulllength | 2993 | NM_002099; glycophorin A precursor |
| 84 | Hs.73828.1.S1 | GenBank | Hs.73828 | fulllength | 6886 | NM_003189; T-cell acute lymphocytic leukemia 1 |
| 85 | g4505220 | RefSeq | Hs.73862 | fulllength | 4317 | NM_002424; matrix metalloproteinase 8 preproprotein |
| 86 | Hs.97469.0_RC | GenBank | Hs.97469 | fulllength | 138121 | |
| 87 | Hs.84522.0.A1 | GenBank | Hs.84522 | fulllength | 148534 | NM_152487; hypothetical protein FLJ31842 |
| 88 | Hs.107526.0_RC | GenBank | Hs.107526 | fulllength | 9334 | NM_004776; UDP-Gal: betaGlcNAc beta 1,4-galactosyltransferase 5 |
| 89 | Hs.99807.0.A1 | GenBank | Hs.99807 | fulllength | 150468 | NM_152515; hypothetical protein FLJ40629 |
| 90 | g6633804 | RefSeq | Hs.247921 | fulllength | 3049 | NM_005331; theta 1 globin |
| 91 | g602433 | GenBank | Hs.187958 | fulllength | 6535 | NM_005629; solute carrier family 8 (neurotransmitter transporter, |
| | | | | | | creatine), member 8 |
| 92 | g189435 | GenBank | Hs.733 | fulllength | 2038 | NM_000119; erythrocyte membrane protein band 4.2 |
| 93 | Hs.298161.3 | GenBank | Hs.356717 | fulllength | 4635 | NM_002476; atrial/embryonic alkali myosin light chain |
| 94 | g7959850_RC | GenBank | Hs.356717 | fulllength | 4635 | NM_002476; atrial/embryonic alkali myosin light chain |
| 95 | g12003206 | GenBank | Hs.197298 | fulllength | 10625 | NM_006469; influenza virus NS1A binding protein |
| 96 | g6806896 | RefSeq | Hs.76930 | fulllength | 6622 | NM_000345; alpha-synuclein isoform NACP140 NM_007308; synuclein, |
| | | | | | | alpha |
| 97 | g4758711 | RefSeq | Hs.122785 | fulllength | 8972 | NM_004668; maltase-glucoamylase |
| 98 | Hs.184019.1.S1 | GenBank | Hs.19385 | fulllength | 51099 | NM_016006; CGI58 protein |
| 99 | Hs.8688.0.A1 | GenBank | Hs.406087 | est | | |
| ## | g7706179 | RefSeq | Hs.274309 | fulllength | 51327 | NM_016633; erythroid associated factor |

TABLE 36
MDS_AML_Blasten

| # | affy id | HUGO name | F | p | q | Title | MapLocation | Sequence Type | Transcript ID | Sequence Derived From |
|---|---|---|---|---|---|---|---|---|---|---|
| 1 | 203919_at | TCEA2 | 87.95 | 3.35e−15 | 1.26e−11 | transcription elongation factor A (SII), 2 | 20q13.33 | Exemplarsequence | Hs.80598.0 | NM_003195.1 |
| 2 | 201061_s_at | STOM | 76.83 | 1.28e−12 | 8.01e−10 | stomatin | 9q34.1 | Exemplarsequence | Hs.160483.0 | M81635.1 |
| 3 | 206674_at | FLT3 | 68.74 | 3.30e−16 | 2.47e−12 | fms-related tyrosine kinase 3 | 13q12 | Exemplarsequence | Hs.385.0 | NM_004119.1 |
| 4 | 212531_at | LCN2 | 64.93 | 1.92e−11 | 4.65e−09 | lipocalin 2 (oncogene 24p3) | 9q34 | Consensussequence | Hs.204238.0 | NM_005564.1 |
| 5 | 201060_x_at | STOM | 58.69 | 4.22e−11 | 6.60e−09 | stomatin | 9q34.1 | Consensussequence | Hs.160483.0 | AI537887 |
| 6 | 201416_at | SOX4 | 58.36 | 1.36e−13 | 2.55e−10 | SRY (sex determining region Y)-box 4 | 6p22.3 | Consensussequence | Hs.83484.0 | NM_003107.1 |
| 7 | 206676_at | CEACAM8 | 58.30 | 3.04e−11 | 5.80e−09 | carcinoembryonic antigen-related cell | 19q13.2 | Exemplarsequence | Hs.41.0 | M33326.1 |
| | | | | | | adhesion molecule 8 | | | | |
| 8 | 221517_s_at | CRSP6 | 58.10 | 3.70e−13 | 3.47e−10 | cofactor required for Sp1 | 11q14 | Exemplarsequence | Hs.22630.1 | AF105421.1 |
| | | | | | | transcriptional activation, subunit 6, | | | | |
| | | | | | | 77 kDa | | | | |
| 9 | 201029_s_at | CD99 | 57.51 | 1.83e−12 | 8.59e−10 | CD99 antigen | Xp22.32 | Exemplarsequence | Hs.177543.0 | NM_002414.1 |
| 10 | 207992_s_at | AMPD3 | 56.54 | 5.83e−12 | 2.08e−09 | adenosine monophosphate | 11p15 | Exemplarsequence | Hs.83918.0 | NM_000480.1 |
| | | | | | | deaminase (isoform E) | | | | |
| 11 | 201417_at | | 55.29 | 3.64e−13 | 3.47e−10 | Homo sapiens mRNA full length | | Consensussequence | Hs.83484.0 | NM_003107.1 |
| | | | | | | insert cDNA clone EUROIMAGE | | | | |
| | | | | | | 1977059 | | | | |
| 12 | 209771_x_at | CD24 | 52.97 | 6.51e−11 | 9.22e−09 | CD24 antigen (small cell lung | 6q21 | Consensussequence | Hs.286124.0 | AA761181 |
| | | | | | | carcinoma cluster 4 antigen) | | | | |
| 13 | 225570_at | SLC41A1 | 52.87 | 1.33e−14 | 3.31e−11 | solute carrier family 41, member 1 | 1q32.1 | Consensussequence | Hs.20274.0 | AW439816 |
| 14 | 203535_at | S100A9 | 52.42 | 1.59e−11 | 4.42e−09 | S100 calcium binding protein A9 | 1q21 | Exemplarsequence | Hs.112405.0 | NM_002965.2 |
| | | | | | | (calgranulin B) | | | | |
| 15 | 216379_x_at | KIAA1919 | 50.55 | 9.72e−11 | 1.12e−08 | KIAA1919 protein | 6q22 | Consensussequence | Hs.332045.0 | AK000168.1 |
| 16 | 225605_at | LOC90313 | 50.27 | 1.80e−12 | 8.59e−10 | hypothetical protein BC004507 | 17q11.1 | Consensussequence | Hs.97871.0 | AL540867 |
| 17 | 207721_x_at | HINT1 | 50.26 | 5.33e−12 | 2.00e−09 | histidine triad nucleotide binding | 5q31.2 | Exemplarsequence | Hs.256697.0 | NM_005340.1 |
| | | | | | | protein 1 | | | | |
| 18 | 214501_s_at | H2AFY | 49.78 | 2.02e−13 | 2.55e−10 | H2A histone family, member Y | 5q31.3-q32 | Consensussequence | Hs.75258.2 | AF044286.1 |
| 19 | 202018_s_at | LTF | 48.30 | 3.50e−11 | 5.80e−09 | lactotransferrin | 3q21-q23 | Exemplarsequence | Hs.105938.0 | NM_002343.1 |
| 20 | 217988_at | HEI10 | 48.15 | 4.48e−11 | 6.72e−09 | enhancer of invasion 10 | 14q11.1 | Exemplarsequence | Hs.107003.0 | NM_021178.1 |
| 21 | 266_s_at | CD24 | 47.82 | 1.99e−10 | 1.94e−08 | CD24 antigen (small cell lung | 6q21 | Exemplarsequence | L33930 | L33930 |
| | | | | | | carcinoma cluster 4 antigen) | | | | |
| 22 | 225029_at | | 47.35 | 6.30e−12 | 2.15e−09 | ESTs | | Consensussequence | Hs.74111.2 | BE958068 |
| 23 | 235663_at | | 46.61 | 5.71e−11 | 8.40e−09 | ESTs | | Consensussequence | Hs.155780.0 | AI313160 |
| 24 | 213666_at | 38961 | 46.43 | 1.85e−11 | 4.64e−09 | septin 6 | Xq24 | Consensussequence | Hs.90998.1 | AK026589.1 |
| 25 | 204351_at | S100P | 46.15 | 2.55e−10 | 2.28e−08 | S100 calcium binding protein P | 4p16 | Exemplarsequence | Hs.2962.0 | NM_005980.1 |
| 26 | 228031_at | | 45.40 | 4.54e−10 | 3.36e−08 | Homo sapiens cDNA FLJ31616 fis, | | Consensussequence | Hs.226666.0 | AW444778 |
| | | | | | | clone NT2RI2003019. | | | | |
| 27 | 202428_x_at | DBI | 45.11 | 3.64e−11 | 5.80e−09 | diazepam binding inhibitor (GABA receptor modulator, acyl- | 2q12-q21 | Exemplarsequence | Hs.78888.0 | NM_020548.1 |
| | | | | | | Coenzyme A binding protein) | | | | |
| 28 | 223671_x_at | FLJ20139 | 45.04 | 3.62e−11 | 5.80e−09 | hypothetical protein FLJ20139 | 1p21.1 | Exemplarsequence | Hs.19978.3 | AF248965.1 |
| 29 | 215501_s_at | DUSP10 | 44.96 | 1.25e−11 | 3.61e−09 | dual specificity phosphatase 10 | 1q41 | Consensussequence | Hs.177534.1 | AK022513.1 |
| 30 | 201015_s_at | JUP | 44.56 | 9.13e−12 | 2.96e−09 | junction plakoglobin | 17q21 | Exemplarsequence | Hs.2340.0 | NM_021991.1 |
| 31 | 217631_at | CRFG | 44.20 | 2.98e−11 | 5.80e−09 | G protein-binding protein CRFG | 10p15-p14 | Consensussequence | Hs.315170.0 | AI081107 |
| 32 | 201937_s_at | DNPEP | 44.12 | 9.48e−12 | 2.96e−09 | aspartyl aminopeptidase | 2q36.1 | Exemplarsequence | Hs.258551.0 | NM_012100.1 |
| 33 | 210254_at | MS4A3 | 43.88 | 2.50e−10 | 2.28e−08 | membrane-spanning 4-domains, | 11q12 | Exemplarsequence | Hs.99960.0 | L35848.1 |
| | | | | | | subfamily A, member 3 | | | | |
| | | | | | | (hematopoietic cell-specific) | | | | |
| 34 | 213668_s_at | SOX4 | 43.59 | 1.05e−11 | 3.14e−09 | SRY (sex determining region Y)-box 4 | 6p22.3 | Consensussequence | Hs.83484.1 | AI989477 |
| 35 | 221563_at | DUSP10 | 43.49 | 3.04e−11 | 5.80e−09 | dual specificity phosphatase 10 | 1q41 | Consensussequence | Hs.177534.0 | N36770 |
| 36 | 228131_at | ASE-1 | 42.97 | 1.10e−10 | 1.17e−08 | CD3-epsilon-associated protein; | 19q13.3 | Consensussequence | Hs.25523.0 | BG111047 |
| | | | | | | antisense to ERCC-1 | | | | |
| 37 | 223446_s_at | DTNBP1 | 42.84 | 2.04e−13 | 2.55e−10 | dystrobrevin binding protein 1 | 6p22.3 | Exemplarsequence | Hs.43481.0 | AF061734.1 |
| 38 | 205349_at | GNA15 | 42.43 | 7.15e−11 | 9.93e−09 | guanine nucleotide binding protein (G | 19p13.3 | Exemplarsequence | Hs.73797.0 | NM_002068.1 |
| | | | | | | protein), alpha 15 (Gq class) | | | | |
| 39 | 224196_x_at | CGI-30 | 42.22 | 6.08e−11 | 8.77e−09 | CGI-30 protein | 1p21.1 | Exemplarsequence | Hs.19978.4 | AF161492.1 |
| 40 | 204234_s_at | ZNF195 | 41.98 | 2.20e−11 | 4.72e−09 | zinc finger protein 195 | 11p15.5 | Consensussequence | Hs.104382.0 | AI476267 |
| 41 | 226043_at | AGS3 | 41.96 | 8.22e−11 | 1.01e−08 | activator of G-protein signaling 3 | 9q34.3 | Consensussequence | Hs.239370.0 | AI242661 |
| 42 | 228562_at | RINZF | 41.50 | 3.34e−11 | 5.80e−09 | zinc finger protein RINZF | 8q13.q21.1 | Consensussequence | Hs.39982.0 | N29918 |
| 43 | 216705_s_at | ADA | 41.32 | 3.34e−11 | 5.80e−09 | adenosine deaminase | 20q12-q13.11 | Consensussequence | Hs.1217.1 | X02189 |
| 44 | 200036_s_at- | RPL10A | 41.31 | 6.70e−10 | 4.31e−08 | ribosomal protein L10a | 6p21.3-p21.2 | Exemplarsequence | Hs.252574.0 | NM_007104.2 |
| | HG-U133A | | | | | | | | | |
| 45 | 211070_x_at | DBI | 41.19 | 1.07e−10 | 1.17e−08 | diazepam binding inhibitor (GABA | 2q12-q21 | Exemplarsequence | g13623678 | BC006466.1 |
| | | | | | | receptor modulator, acyl-Coenzyme | | | | |
| | | | | | | A binding protein) | | | | |
| 46 | 207802_at | SGP28 | 41.18 | 1.88e−09 | 9.67e−08 | specific granule protein (28 kDa) | 6p12.3 | Exemplarsequence | Hs.54431.0 | NM_006061.1 |
| 47 | 225831_at | LOC148894 | 41.16 | 3.02e−11 | 5.80e−09 | hypothetical protein LOC148894 | 1p36.11 | Consensussequence | Hs.154085.0 | AK025345.1 |
| 48 | 235753_at | | 41.15 | 3.63e−12 | 1.43e−09 | Homo sapiens cDNA FLJ34835 fis, | | Consensussequence | Hs.196169.0 | AI492051 |
| | | | | | | clone NT2NE2010150. | | | | |
| 49 | 202391_at | BASP1 | 40.94 | 6.72e−10 | 4.31e−08 | brain abundant, membrane attached | 5p15.1-p14 | Exemplarsequence | Hs.79516.0 | NM_006317.1 |
| | | | | | | signal protein 1 | | | | |
| 50 | 219218_at | FLJ23058 | 40.83 | 2.02e−11 | 4.72e−09 | hypothetical protein FLJ23058 | 17q25.3 | Exemplarsequence | Hs.98968.0 | NM_024696.1 |
| 51 | 208890_s_at | PLXNB2 | 40.29 | 2.14e−10 | 2.05e−08 | plexin B2 | 22q13.33 | Exemplarsequence | Hs.3989.0 | BC004542.1 |
| 52 | 220974_x_at | BA108L7.2 | 39.97 | 7.97e−11 | 1.01e−08 | similar to rat tricarboxylate carrier-like | 10q24.31 | Exemplarsequence | g13569945 | NM_030971.1 |
| | | | | | | protein | | | | |
| 53 | 208943_s_at | TLOC1 | 39.80 | 8.11e−10 | 5.03e−08 | translocation protein 1 | 3q26.2-q27 | Exemplarsequence | Hs.8146.0 | U93239.1 |
| 54 | 212746_s_at | KIAA0470 | 39.25 | 2.14e−11 | 4.72e−09 | KIAA0470 gene product | 1q44 | Consensussequence | Hs.25132.3 | AA126789 |
| 55 | 205474_at | CRLF3 | 39.23 | 1.54e−09 | 8.29e−08 | cytokine receptor-like factor 3 | 17q11.2 | Exemplarsequence | Hs.7120.0 | NM_015986.1 |
| 56 | 201268_at | NME2 | 39.13 | 3.81e−10 | 3.05e−08 | non-metastatic cells 2, protein | 17q21.3 | Exemplarsequence | Hs.275163.0 | NM_002512.1 |
| | | | | | | (NM23B) expressed in | | | | |
| 57 | 201418_s_at | SOX4 | 38.97 | 3.39e−12 | 1.43e−09 | SRY (sex determining region Y)-box 4 | 6p22.3 | Exemplarsequence | Hs.83484.0 | NM_003107.1 |
| 58 | 212242_at | TUBA1 | 38.91 | 1.22e−09 | 6.91e−08 | tubulin, alpha 1 (testis specific) | 2q36.1 | Consensussequence | Hs.75318.0 | AL565074 |
| 59 | 224733_at | CKLFSF3 | 38.62 | 1.09e−10 | 1.17e−08 | chemokine-like factor super family 3 | 16q21 | Consensussequence | Hs.7773.4 | AL574900 |
| 60 | 216650_at | | 38.49 | 9.61e−13 | 6.55e−10 | | | Consensussequence | Hs.166181.0 | AL008627 |
| 61 | 208650_s_at | CD24 | 38.29 | 2.48e−09 | 1.19e−07 | CD24 antigen (small cell lung | 6q21 | Consensussequence | Hs.286124.1 | BG327863 |
| | | | | | | carcinoma cluster 4 antigen) | | | | |
| 62 | 211657_at | CEACAM6 | 38.13 | 2.37e−09 | 1.15e−07 | carcinoembryonic antigen-related cell | 19q13.2 | Exemplarsequence | g189084 | M18728.1 |
| | | | | | | adhesion molecule 6 (non-specific | | | | |
| | | | | | | cross reacting antigen) | | | | |
| 63 | 224928_at | SET7 | 37.95 | 1.25e−10 | 1.31e−08 | SET domain-containing protein 7 | 4q28 | Consensussequence | Hs.78521.0 | AK024846.1 |
| 64 | 225877_at | MGC34695 | 37.93 | 7.41e−13 | 5.56e−10 | hypothetical protein MGC34695 | 10q22.1 | Consensussequence | Hs.15386.0 | BG389635 |
| 65 | 211031_s_at | CYLN2 | 37.76 | 2.32e−10 | 2.17e−08 | cytoplasmic linker 2 | 7q11.23 | Exemplarsequence | g13623312 | BC006259.1 |
| 66 | 227056_at | | 37.55 | 1.26e−10 | 1.31e−08 | ESTs | | Consensussequence | Hs.63510.1 | AA181172 |
| 67 | 204639_at | ADA | 37.54 | 4.42e−11 | 6.72e−09 | adenosine deaminase | 20q12-q13.11 | Exemplarsequence | Hs.1217.0 | NM_000022.1 |
| 68 | 205997_at | ADAM28 | 37.51 | 3.25e−10 | 2.80e−08 | a disintegrin and metalloproteinase | 8p21.1 | Exemplarsequence | Hs.174030.0 | NM_021778.1 |
| | | | | | | domain 28 | | | | |
| 69 | 201063_at | RCN1 | 37.32 | 8.07e−11 | 1.01e−08 | reticulocalbin 1, EF-hand calcium | 11p13 | Exemplarsequence | Hs.167791.0 | NM_002901.1 |
| | | | | | | binding domain | | | | |
| 70 | 230302_at | | 37.26 | 7.93e−11 | 1.01e−08 | Homo sapiens full length insert cDNA | | Consensussequence | Hs.264606.0 | AI741597 |
| | | | | | | clone ZD68B12 | | | | |
| 71 | 201536_at | DUSP3 | 37.23 | 4.62e−10 | 3.37e−08 | dual specificity phosphatase 3 | 17q21 | Consensussequence | Hs.181046.0 | AL048503 |
| | | | | | | (vaccinia virus phosphatase VH1- | | | | |
| | | | | | | related) | | | | |
| 72 | 225510_at | | 37.08 | 6.56e−13 | 5.47e−10 | Homo sapiens cDNA FLJ11658 fis, | | Consensussequence | Hs.22660.1 | BG033561 |
| | | | | | | clone HEMBA1004577. | | | | |
| 73 | 219590_x_at | CGI-30 | 36.97 | 5.57e−10 | 3.80e−08 | CGI-30 protein | 1p21.1 | Exemplarsequence | Hs.19978.0 | NM_015958.1 |
| 74 | 209389_x_at | DBI | 36.42 | 4.16e−10 | 3.15e−08 | diazepam binding inhibitor (GABA | 2q12-q21 | Exemplarsequence | Hs.78888.1 | M15887.1 |
| | | | | | | receptor modulator, acyl-Coenzyme | | | | |
| | | | | | | A binding protein) | | | | |
| 75 | 242051_at | | 36.40 | 1.05e−10 | 1.17e−08 | ESTs | | Consensussequence | Hs.130260.0 | AI695695 |
| 76 | 235142_at | MGC17919 | 36.38 | 1.57e−10 | 1.57e−08 | hypothetical protein MGC17919 | 1p34.3 | Consensussequence | Hs.129837.0 | AW006067 |
| 77 | 209369_at | ANXA3 | 36.27 | 4.25e−09 | 1.75e−07 | annexin A3 | 4q13-q22 | Exemplarsequence | Hs.1378.0 | M63310.1 |
| 78 | 221778_at | KIAA1718 | 36.21 | 4.53e−09 | 1.85e−07 | KIAA1718 protein | 7q33-q35 | Consensussequence | Hs.222707.0 | BE217882 |
| 79 | 224916_at | | 36.20 | 2.19e−11 | 4.72e−09 | Homo sapiens cDNA FLJ38577 fis, | | Consensussequence | Hs.86347.1 | BG286973 |
| | | | | | | clone HCHON2007650. | | | | |
| 80 | 221754_s_at | CORO1B | 36.18 | 1.82e−12 | 8.59e−10 | coronin, actin-binding protein, 1B | 11q13.1 | Consensussequence | Hs.6191.0 | AI341234 |
| 81 | 221726_at | RPL22 | 36.07 | 1.23e−09 | 6.91e−08 | ribosomal protein L22 | 1p36.3-p36.2 | Consensussequence | Hs.326249.1 | BE250348 |
| 82 | 207269_at | DEFA4 | 36.02 | 8.40e−10 | 5.17e−08 | defensin, alpha 4, corticostatin | 8p23 | Exemplarsequence | Hs.2582.0 | NM_001925.1 |
| 83 | 203765_at | GCA | 35.96 | 5.09e−09 | 1.99e−07 | grancalcin, EF-hand calcium binding | 2q24.3 | Exemplarsequence | Hs.79381.0 | NM_012198.1 |
| | | | | | | protein | | | | |
| 84 | 208651_x_at | CD24 | 35.93 | 3.83e−09 | 1.64e−07 | CD24 antigen (small cell lung | 6q21 | Exemplarsequence | Hs.286124.1 | M58664.1 |
| | | | | | | carcinoma cluster 4 antigen) | | | | |
| 85 | 232884_s_at | DKFZp434J1015 | 35.87 | 2.26e−10 | 2.14e−08 | hypothetical protein DKFZp434J1015 | 7p22.2 | Consensussequence | Hs.283779.0 | AL133055.1 |
| 86 | 209310_s_at | CASP4 | 35.80 | 7.58e−11 | 1.01e−08 | caspase 4, apoptosis-related | 11q22.2-q22.3 | Exemplarsequence | Hs.74122.0 | U25804.1 |
| | | | | | | cysteine protease | | | | |
| 87 | 205863_at | S100A12 | 35.58 | 3.98e−09 | 1.69e−07 | S100 calcium binding protein A12 | 1q21 | Exemplarsequence | Hs.19413.0 | NM_005621.1 |
| | | | | | | (calgranulin C) | | | | |
| 88 | 221781_s_at | ERdj5 | 35.56 | 4.81e−10 | 3.43e−08 | ER-resident protein ERdj5 | 2q32.1 | Consensussequence | Hs.1098.0 | AL137648.1 |
| 89 | 239562_at | | 35.55 | 4.71e−10 | 3.40e−08 | Homo sapiens, clone | | Consensussequence | Hs.136330.0 | AW272411 |
| | | | | | | IMAGE: 4830703, mRNA, partial cds | | | | |
| 90 | 214298_x_at | 38961 | 35.40 | 3.41e−10 | 2.87e−08 | septin 6 | Xq24 | Consensussequence | Hs.90998.3 | AL568374 |
| 91 | 203213_at | CDC2 | 35.37 | 3.87e−09 | 1.65e−07 | cell division cycle 2, G1 to S and G2 | 10q21.1 | Consensussequence | Hs.184572.0 | AL524035 |
| | | | | | | to M | | | | |
| 92 | 225615_at | LOC126917 | 35.33 | 7.26e−10 | 4.58e−08 | hypothetical protein LOC126917 | 1p36.13 | Consensussequence | Hs.13766.0 | AK024480.1 |
| 93 | 223276_at | NID67 | 35.30 | 2.59e−10 | 2.29e−08 | putative small membrane protein | 5q33.1 | Exemplarsequence | Hs.29444.0 | AF313413.1 |
| | | | | | | NID67 | | | | |
| 94 | 215116_s_at | DNM1 | 35.19 | 1.33e−10 | 1.37e−08 | dynamin 1 | 9q34 | Consensussequence | Hs.166161.2 | AF035321.1 |
| 95 | 209889_at | SEC31B-1 | 35.18 | 8.68e−11 | 1.05e−08 | secretory pathway component | 10q24.2 | Exemplarsequence | Hs.18889.0 | AF274863.1 |
| | | | | | | Sec31B-1 | | | | |
| 96 | 211323_s_at | ITPR1 | 35.18 | 1.75e−11 | 4.52e−09 | inositol 1,4,5-triphosphate receptor, | 3p26-p25 | Exemplarsequence | Hs.198443.1 | L38019.1 |
| | | | | | | type 1 | | | | |
| 97 | 220755_s_at | C6orf48 | 35.03 | 1.10e−09 | 6.50e−08 | chromosome 6 open reading frame | 6p21.3 | Exemplarsequence | Hs.109798.0 | NM_016947.1 |
| | | | | | | 48 | | | | |
| 98 | 224710_at | RAB34 | 34.95 | 6.64e−10 | 4.31e−08 | RAB34, member RAS oncogene | 17q11.1 | Consensussequence | Hs.301853.0 | AF322067.1 |
| | | | | | | family | | | | |
| 99 | 221002_s_at | DC-TM4F2 | 34.89 | 5.15e−09 | 2.00e−07 | tetraspanin similar to TM4SF9 | 10q22.3 | Exemplarsequence | g13569888 | NM_030927.1 |
| 100 | 209563_x_at | CALM1 | 34.52 | 6.37e−09 | 2.41e−07 | calmodulin 1 (phosphorylase kinase, | 14q24-q31 | Exemplarsequence | Hs.182278.1 | BC000454.1 |
| | | | | | | delta) | | | | |

| # | Sequence ID | Sequence Source | Unigene_Accession | Cluster_Type | LocusLink | Full_Length_Reference_Seq |
|---|---|---|---|---|---|---|
| 1 | g4507384 | RefSeq | Hs.80598 | fulllength | 6919 | NM_003195; transcription elongation factor A (SII), 2 |
| 2 | g181183 | GenBank | Hs.160483 | fulllength | 2040 | NM_004099; stomatin |
| 3 | g4758395 | RefSeq | Hs.385 | fulllength | 2322 | NM_004119; fms-related tyrosine kinase 3 |
| 4 | Hs.204238.0.S1 | GenBank | Hs.204238 | fulllength | 3934 | NM_005564; lipocalin 2 (oncogene 24p3) |
| 5 | Hs.160483.0 | GenBank | Hs.160483 | fulllength | 2040 | NM_004099; stomatin |
| 6 | Hs.83484.0 | GenBank | Hs.83484 | fulllength | 6659 | NM_003107; SRY (sex determining region Y)-box 4 |
| 7 | g189101 | GenBank | Hs.41 | fulllength | 1088 | NM_001816; carcinoembryonic antigen-related cell adhesion molecule 8 |
| 8 | g4838128 | GenBank | Hs.22630 | fulllength | 9440 | NM_004268; cofactor required for Sp1 transcriptional activation, subunit 6, 77 kDa |
| 9 | g4505182 | RefSeq | Hs.433387 | fulllength | 4267 | NM_002414; CD99 antigen |
| 10 | g4502078 | RefSeq | Hs.83918 | fulllength | 272 | NM_000480; adenosine monophosphate deaminase (isoform E) |
| 11 | Hs.83484.0 | GenBank | Hs.351928 | | | |
| 12 | Hs.286124.0_RC | GenBank | Hs.375108 | fulllength | 934 | NM_013230; CD24 antigen (small cell lung carcinoma cluster 4 antigen) |
| 13 | Hs.20274.0_RC | GenBank | Hs.20274 | fulllength | 254428 | NM_173854; solute carrier family 41 member 1 |
| 14 | g9845520 | RefSeq | Hs.112405 | fulllength | 6280 | NM_002965; S100 calcium-binding protein A9 |
| 15 | Hs.332045.0_RC | GenBank | Hs.400572 | fulllength | 91749 | NM_153369; KIAA1919 protein |
| 16 | Hs.97871.0.S1 | GenBank | Hs.97871 | fulllength | 90313 | |
| 17 | g4885412 | RefSeq | Hs.256697 | fulllength | 3094 | NM_005340; histidine triad nucleotide binding protein 1 |
| 18 | Hs.75258.2 | GenBank | Hs.75258 | fulllength | 9555 | NM_004893; H2A histone family, member Y isoform 2 NM_138609; H2A histone family, member Y isoform 1 |
| | | | | | | NM_138610; H2A histone family, member Y isoform 3 |
| 19 | g4505042 | RefSeq | Hs.105938 | fulllength | 4057 | NM_002343; lactotransferrin |
| 20 | g10863978 | RefSeq | Hs.107003 | fulllength | 57820 | NM_021178; enhancer of invasion 10 |
| 21 | L33930 | GenBank | Hs.375108 | fulllength | 934 | NM_013230; CD24 antigen (small cell lung carcinoma cluster 4 antigen) |
| 22 | Hs.74111.2 | GenBank | Hs.355559 | est | | |
| 23 | Hs.155780.0.A1 | GenBank | Hs.436070 | est | | |
| 24 | Hs.90998.1.S1 | GenBank | Hs.90998 | fulllength | 23157 | NM_015129; septin 6 isoform B NM_145799; septin 6 isoform A NM_145800; |
| | | | | | | septin 6 isoform A NM_145802; septin 6 isoform D |
| 25 | g5174662 | RefSeq | Hs.2962 | fulllength | 6286 | NM_005980; S100 calcium binding protein P |
| 26 | Hs.226666.0.A1 | GenBank | Hs.226666 | | | |
| 27 | g10140852 | RefSeq | Hs.78888 | fulllength | 1622 | NM_020548; diazepam binding inhibitor |
| 28 | g12005666 | GenBank | Hs.406051 | fulllength | 54833 | NM_017685; hypothetical protein FLJ20139 |
| 29 | Hs.177534.1 | GenBank | Hs.177534 | fulllength | 11221 | NM_007207; dual specificity phosphatase 10 isoform a NM_144728; dual specificity phosphatase 10 isoform b |
| | | | | | | NM_144729; dual specificity phosphatase 10 isoform b |
| 30 | g12056467 | RefSeq | Hs.2340 | fulllength | 3728 | NM_002230; junction plakoglobin NM_021991; junction plakoglobin |
| 31 | Hs.315170.0.A1 | GenBank | Hs.215766 | fulllength | 23560 | NM_012341; G protein-binding protein CRFG |
| 32 | g6912247 | RefSeq | Hs.258551 | fulllength | 23549 | NM_012100; aspartyl aminopeptidase |
| 33 | g561638 | GenBank | Hs.99960 | fulllength | 932 | NM_006138; membrane-spanning 4-domains, subfamily A, member 3 |
| 34 | Hs.83484.1 | GenBank | Hs.83484 | fulllength | 6659 | NM_003107; SRY (sex determining region Y)-box 4 |
| 35 | Hs.177534.0 | GenBank | Hs.177534 | fulllength | 11221 | NM_007207; dual specificity phosphatase 10 isoform a NM_144728; dual |
| | | | | | | specificity phosphatase 10 isoform b NM_144729; dual specificity phosphatase 10 |
| | | | | | | isoform b |
| 36 | Hs.25523.0_RC | GenBank | Hs.211956 | fulllength | 10849 | NM_012099; CD3-epsilon-associated protein; antisense to ERCC-1 |
| 37 | g12002003 | GenBank | Hs.43481 | fulllength | 84062 | NM_032122; dystrobrevin binding protein 1 |
| 38 | g4504038 | RefSeq | Hs.73797 | fulllength | 2769 | NM_002068; guanine nucleotide binding protein (G protein), alpha 15 (Gq class) |
| 39 | g6841507 | GenBank | Hs.441133 | fulllength | 51611 | NM_015958; CGI-30 protein |
| 40 | Hs.104382.0 | GenBank | Hs.104382 | fulllength | 7748 | NM_007152; zinc finger protein 195 |
| 41 | Hs.239370.0.S1 | GenBank | Hs.239370 | fulllength | 26086 | NM_015597; activator of G-protein signaling 3 |
| 42 | Hs.39982.0_RC | GenBank | Hs.237146 | fulllength | 65986 | NM_023929; zinc finger protein RINZF |
| 43 | Hs1217.1 | GenBank | Hs.1217 | fulllength | 100 | NM_000022; adenosine deaminase |
| 44 | g6325471 | RefSeq | Hs.425293 | fulllength | 4736 | NM_007104; ribosomal protein L10a |
| 45 | g13623678 | GenBank | Hs.78888 | fulllength | 1622 | NM_020548; diazepam binding inhibitor |
| 46 | g5174674 | RefSeq | Hs.54431 | fulllength | 10321 | NM_006061; specific granule protein (28 kDa) |
| 47 | Hs.154085.0_RC | GenBank | Hs.348515 | fulllength | 148894 | |
| 48 | Hs.196169.0 | GenBank | Hs.196169 | | | |
| 49 | g5453749 | RefSeq | Hs.79516 | fulllength | 10409 | NM_006317; brain abundant, membrane attached signal protein 1 |
| 50 | g13375978 | RefSeq | Hs.98968 | fulllength | 79749 | NM_024696; hypothetical protein FLJ23058 |
| 51 | g13528689 | GenBank | Hs.3989 | fulllength | 23654 | NM_012401; plexin B2 |
| 52 | g13569945 | RefSeq | Hs.283844 | fulllength | 81855 | NM_030971; similar to rat tricarboxylate carrier-like protein |
| 53 | g1928972 | GenBank | Hs.8146 | fulllength | 7095 | NM_003262; translocation protein 1 |
| 54 | Hs.25132.3_RC | GenBank | Hs.25132 | fulllength | 9859 | NM_014812; KARP-1-binding protein |
| 55 | g7705331 | RefSeq | Hs.7120 | fulllength | 51379 | NM_015986; cytokine receptor-like factor 3 |
| 56 | g4505408 | RefSeq | Hs.433416 | fulllength | 4831 | NM_002512; non-metastatic cells 2, protein (NM238) expressed in |
| 57 | g4507162 | RefSeq | Hs.83484 | fulllength | 6659 | NM_003107; SRY (sex determining region Y)-box 4 |
| 58 | Hs.75318.0 | GenBank | Hs.75318 | fulllength | 7277 | NM_006000; tubulin, alpha 1 |
| 59 | Hs.7773.4_RC | GenBank | Hs.7773 | fulllength | 123920 | NM_144601; chemokine-like factor super family 3 isoform a |
| 60 | Hs.166181.0 | GenBank | | | | |
| 61 | Hs.286124.1_RC | GenBank | Hs.375108 | fulllength | 934 | NM_013230; CD24 antigen (small cell lung carcinoma cluster 4 antigen) |
| 62 | g189084 | GenBank | Hs.73848 | fulllength | 4680 | NM_002483; carcinoembryonic antigen-related cell adhesion molecule 6 (non- |
| | | | | | | specific cross reacting antigen) |
| 63 | Hs.78521.0.S1 | GenBank | Hs.78521 | fulllength | 80854 | NM_030648; SET domain-containing protein 7 |
| 64 | Hs.15386.0.A1 | GenBank | Hs.15386 | fulllength | 219743 | NM_173555; hypothetical protein MGC34695 |
| 65 | g13623312 | GenBank | Hs.104717 | fulllength | 7461 | NM_003388; cytoplasmic linker 2 isoform 1 NM_032421; cytoplasmic linker 2 |
| | | | | | | isoform 2 NM_032719; |
| 66 | Hs.63510.1.A1 | GenBank | Hs.446502 | est | | |
| 67 | g4557248 | RefSeq | Hs.1217 | fulllength | 100 | NM_000022; adenosine deaminase |
| 68 | g11496995 | RefSeq | Hs.174030 | fulllengh | 10863 | NM_014265; a disintegrin and metalloproteinase domain 28 isoform 1 |
| | | | | | | preproprotein NM_021777; a disintegrin and metalloproteinase domain 28 isoform |
| | | | | | | 3 preproprotein NM_021778; a disintegrin and metalloproteinase domain 28 |
| | | | | | | isoform 2 preproprotein |
| 69 | g4506454 | RefSeq | Hs.167791 | fulllength | 5954 | NM_002901; reticulocalbin 1 precursor |
| 70 | Hs.264606.0_RC | GenBank | Hs.264606 | | | |
| 71 | Hs.181046.0_RC | GenBank | Hs.181046 | fulllength | 1845 | NM_004090; dual specificity phosphatase 3 |
| 72 | Hs.22660.1_RC | GenBank | Hs.380824 | | | |
| 73 | g7706271 | RefSeq | Hs.441133 | fulllength | 51611 | NM_015958; CGI-30 protein |
| 74 | g181960 | GenBank | Hs.78888 | fulllength | 1622 | NM_020548; diazepam binding inhibitor |
| 75 | Hs.130260.0_RC | GenBank | Hs.130260 | est | | |
| 76 | Hs.129837.0_RC | GenBank | Hs.129837 | fulllenglh | 127557 | NM_144621; hypothetical protein MGC17919 |
| 77 | g178696 | GenBank | Hs.1378 | fulllength | 306 | NM_005139: annexin A3 |
| 78 | Hs.222707.0.A2 | GenBank | Hs.222707 | | 80853 | |
| 79 | Hs.86347.1_RC | GenBank | Hs.379754 | | | |
| 80 | Hs.6191.0.S1 | GenBank | Hs.6191 | fulllength | 57175 | NM_020441; coronin, actin binding protein, 1B |
| 81 | Hs.326249.1.A2 | GenBank | Hs.326249 | fulllength | 6146 | NM_000983; ribosomal protein L22 proprotein |
| 82 | g4503302 | RefSep | Hs.2582 | fulllength | 1669 | NM_001925; defensin, alpha 4, preproprotein |
| 83 | g6912387 | RefSeq | Hs.79381 | fulllength | 25801 | NM_012198; grancalcin. EF-hand calcium binding protein |
| 84 | g180167 | GenBank | Hs.375108 | fulllength | 934 | NM_013230; CD24 antigen (small cell lung carcinoma cluster 4 antigen) |
| 85 | Hs.283779.0 | GenBank | Hs.283779 | | 54753 | |
| 86 | g886049 | GenBank | Hs.74122 | fulllength | 837 | NM_001225; caspase 4 isoform alpha precursor NM_033306; caspase 4 isoform |
| | | | | | | gamma precursor NM_033307; caspase 4 isoform delta precursor |
| 87 | g5032058 | RefSeq | Hs.19413 | fulllength | 6283 | NM_005621; S100 calcium-binding protein A12 |
| 88 | Hs.1098.0_RC | GenBank | Hs.1098 | fulllength | 54431 | NM_018981; ER-resident protein ERdj5 |
| 89 | Hs.136330.0.A1 | GenBank | Hs.407156 | | | |
| 90 | Hs.90998.3.S1 | GenBank | Hs.90998 | fulllength | 23157 | NM_015129; septin 6 isoform B NM_145799; septin 6 isoform A NM_145800; |
| | | | | | | septin 6 isoform A NM_145802; septin 6 isoform D |
| 91 | Hs.184572.0.S2 | GenBank | Hs.334562 | fulllength | 983 | NM_001786; cell division cycle 2 protein isoform 1 NM_033379; cell division cycle |
| | | | | | | 2 protein isoform 2 |
| 92 | Hs.13766.0 | GenBank | Hs.13766 | | 126917 | |
| 93 | g12484085 | GenBank | Hs.29444 | fulllength | 85027 | NM_032947; putative small membrane protein NID67 |
| 94 | Hs.166161.2 | GenBank | Hs.166161 | fulllength | 1759 | NM_094408; dynamin 1 |
| 95 | g8650513 | GenBank | Hs.18889 | fulllength | 25956 | NM_015490; secretory pathway component Sec31B-1 |
| 96 | g1464750 | GenBank | Hs.198443 | fulllength | 3708 | NM_002222; inositol 1,4,5-triphosphate receptor, type 1 |
| 97 | g8393383 | RefSeq | Hs.109798 | fulllength | 50854 | NM_016947; G8 protein |
| 98 | Hs.301853.0 | GenBank | Hs.301853 | fulllength | 83871 | NM_031934; RAB39 |
| 99 | g13569858 | RefSeq | Hs.101395 | fulllength | 81619 | NM_030927; tetraspanin similar to TM4SF9 |
| 100 | g12653368 | GenBank | Hs.282410 | fulllength | 801 | NM_006888; calmodulin 1 (phosphorylase kinase, delta) |

TABLE 37
MDS_AML_Blasten

| | | | | | | | | | | Sequence | |
|---|---|---|---|---|---|---|---|---|---|---|---|
| # | affy id | HUGO name | F | p | q | Title | MapLocation | Sequence Type | Transcript ID | Derived From | Sequence ID |
| 1 | 223157_at | MGC3232 | 159.61 | 3.40e−27 | 6.15e−23 | hypothetical protein MGC3232 | 4q12 | Exemplarsequence | Hs.8715.0 | BC004894.1 | g13436154 |
| 2 | 218436_at | SIL1 | 139.91 | 3.10e−24 | 2.80e−20 | endoplasmic reticulum | 5q31 | Exemplarsequence | Hs.297875.0 | NM_022464.1 | g11968008 |
| | | | | | | chaperone SIL1, homolog of | | | | | |
| | | | | | | yeast | | | | | |
| 3 | 201922_at | YR-29 | 127.03 | 1.09e−19 | 4.92e−16 | hypothetical protein YR-29 | 5q13.2 | Exemplarsequence | Hs.8170.0 | NM_014886.1 | g7662676 |
| 4 | 201978_s_at | KIAA0141 | 122.17 | 2.45e−21 | 1.48e−17 | KIAA0141 gene product | 5q31.3 | Exemplarsequence | Hs.63510.0 | NM_014773.1 | g7661939 |
| 5 | 227056_at | | 117.39 | 3.61e−19 | 1.31e−15 | ESTs | | Consensussequence | Hs.63510.1 | AA181172 | Hs.63510.1.A1 |
| 6 | 222229_x_at | | 99.17 | 5.12e−16 | 9.26e−13 | | | Consensussequence | Hs.272344.0 | AL121871 | Hs.272344.0 |
| 7 | 212145_at | MRPS27 | 91.69 | 4.15e−17 | 1.07e−13 | mitochondrial ribosomal protein | 5q13.1 | Consensussequence | Hs.122669.0 | D87453.1 | Hs.122669.0 |
| | | | | | | S27 | | | | | |
| 8 | 201574_at | ETF1 | 91.60 | 6.38e−17 | 1.28e−13 | eukaryotic translation | 5q31.1 | Exemplarsequence | Hs.77324.0 | NM_004730.1 | g4759033 |
| | | | | | | termination factor 1 | | | | | |
| 9 | 224916_at | | 90.13 | 7.98e−18 | 2.41e−14 | Homo sapiens cDNA FLJ38577 | | Consensussequence | Hs.86347.1 | BG286973 | Hs.86347.1_RC |
| | | | | | | fis, clone HCHON2007650. | | | | | |
| 10 | 222983_s_at | PAIP2 | 88.66 | 7.65e−16 | 1.15e−12 | PABP-interacting protein 2 | 5q31.3 | Exemplarsequence | Hs.108548.0 | BC001716.1 | g12804590 |
| 11 | 200093_s_at- | HINT1 | 86.95 | 7.18e−16 | 1.15e−12 | histidine triad nucleotide binding | 5q31.2 | Consensussequence | Hs.256697.2 | N32864 | Hs.256697.2.A1 |
| | HG-U133A | | | | | protein 1 | | | | | |
| 12 | 229693_at | | 85.38 | 5.56e−17 | 1.26e−13 | ESTs | | Consensussequence | Hs.154574.0 | AI952836 | Hs.154574.0_RC |
| 13 | 224936_at | EIF2S3 | 83.13 | 1.45e−15 | 1.87e−12 | eukaryotic translation initiation | Xp22.2-p22.1 | Consensussequence | Hs.30376.1 | BE252813 | Hs.30376.1.A1 |
| | | | | | | factor 2, subunit 3 gamma, | | | | | |
| | | | | | | 52 kDa | | | | | |
| 14 | 214919_s_at | | 81.93 | 1.03e−15 | 1.43e−12 | Homo sapiens, clone | | Consensussequence | Hs.301226.2 | R39094 | Hs.301226.2.A1 |
| | | | | | | IMAGE: 3866125, mRNA | | | | | |
| 15 | 221476_s_at | RPL15 | 81.09 | 4.57e−14 | 3.44e−11 | ribosomal protein L15 | 3p24.1 | Exemplarsequence | Hs.74267.0 | AF279903.1 | g12006349 |
| 16 | 203538_at | CAMLG | 79.11 | 3.43e−15 | 4.13e−12 | calcium modulating ligand | 5q23 | Exemplarsequence | Hs.13572.0 | NM_001745.1 | g4502558 |
| 17 | 223276_at | NID67 | 75.50 | 5.70e−15 | 6.44e−12 | putative small membrane | 5q33.1 | Exemplarsequence | Hs.29444.0 | AF313413.1 | g12484085 |
| | | | | | | protein NID67 | | | | | |
| 18 | 223671_x_at | FLJ20139 | 74.89 | 2.08e−14 | 1.79e−11 | hypothetical protein FLJ20139 | 1p21.1 | Exemplarsequence | Hs.19978.3 | AF248965.1 | g12005666 |
| 19 | 208826_x_at | HINT1 | 74.74 | 5.02e−14 | 3.63e−11 | histidine triad nucleotide binding | 5q31.2 | Exemplarsequence | Hs.256697.1 | U27143.1 | g862932 |
| | | | | | | protein 1 | | | | | |
| 20 | 207721_x_at | HINT1 | 74.67 | 2.83e−14 | 2.22e−11 | histidine triad nucleotide binding | 5q31.2 | Exemplarsequence | Hs.256697.0 | NM_005340.1 | g4885412 |
| | | | | | | protein 1 | | | | | |
| 21 | 208646_at | RPS14 | 74.09 | 2.75e−14 | 2.22e−11 | ribosomal protein S14 | 5q31-q33 | Exemplarsequence | Hs.244621.0 | AF116710.1 | g7959918 |
| 22 | 200074_s_at- | RPL14 | 74.03 | 1.06e−13 | 7.39e−11 | ribosomal protein L14 | 3p22.p21.2 | Exemplarsequence | Hs.738.2 | U16738.1 | g608516 |
| | HG-U133A | | | | | | | | | | |
| 23 | 203024_s_at | HTGN29 | 73.89 | 8.94e−15 | 8.99e−12 | HTGN29 protein | 5q31.1 | Exemplarsequence | Hs.283437.0 | NM_020199.1 | g9910277 |
| 24 | 212894_at | SUPV3L1 | 72.55 | 5.28e−13 | 2.89e−10 | suppressor of var1, 3-like 1 (S. cerevisiae) | 10q22.1 | Consensussequence | Hs.106469.0 | NM_003171.1 | Hs.106469.0 |
| 25 | 35820_at | GM2A | 70.44 | 1.65e−14 | 1.57e−11 | GM2 ganglioside activator | 5q31.3-q33.1 | Consensussequence | 4898374 | X62078 | 4898374 |
| | | | | | | protein | | | | | |
| 26 | 201345_s_at | UBE2D2 | 69.89 | 3.17e−13 | 1.88e−10 | ubiquitin-conjugating enzyme | 5q31.3 | Exemplarsequence | Hs.108332.0 | NM_003339.1 | g4507774 |
| | | | | | | E2D 2 (UBC4/5 homolog, yeast) | | | | | |
| 27 | 213846_at | COX7C | 69.81 | 8.70e−13 | 4.63e−10 | cytochrome c oxidase subunit | 5q14 | Consensussequence | Hs.3462.1 | AA382702 | Hs.3462.1 |
| | | | | | | VIIc | | | | | |
| 28 | 204828_at | RAD9 | 68.11 | 4.20e−12 | 1.81e−09 | RAD9 homolog (S. pombe) | 11q13.1-q13.2 | Exemplarsequence | Hs.240457.0 | NM_004584.1 | g4759021 |
| 29 | 224196_x_at | CGI-30 | 68.02 | 1.31e−13 | 8.75e−11 | CGI-30 protein | 1p21.1 | Exemplarsequence | Hs.19978.4 | AF161492.1 | g6841507 |
| 30 | 202413_s_at | USP1 | 67.39 | 1.21e−12 | 6.18e−10 | ubiquitin specific protease 1 | 1p32.1-p31.3 | Exemplarsequence | Hs.35086.0 | NM_003368.1 | g4507850 |
| 31 | 208652_at | PPP2CA | 67.30 | 3.91e−13 | 2.21e−10 | protein phosphatase 2 (formerly | 5q23-q31 | Exemplarsequence | Hs.91773.0 | BC000400.1 | g12653262 |
| | | | | | | 2A), catalytic subunit, alpha | | | | | |
| | | | | | | isoform | | | | | |
| 32 | 236892_s_at | | 66.64 | 8.65e−15 | 8.99e−12 | Homo sapiens, clone | | Consensussequence | Hs.269918.0 | BF590528 | Hs.269918.0.A1 |
| | | | | | | MGC: 10077 IMAGE: 3896690, | | | | | |
| | | | | | | mRNA, complete cds | | | | | |
| 33 | 218953_s_at | MGC3265 | 66.41 | 1.84e−14 | 1.66e−11 | hypothetical protein MGC3265 | 5q32 | Exemplarsequence | Hs.257111.0 | NM_024028.1 | g13128973 |
| 34 | 217846_at | QARS | 66.06 | 1.49e−12 | 7.26e−10 | glutaminyl-tRNA synthetase | 3p21.3-p21.1 | Exemplarsequence | Hs.79322.0 | NM_005051.1 | g4826959 |
| 35 | 200093_s_at- | HINT1 | 65.91 | 1.23e−12 | 6.18e−10 | histidine triad nucleotide binding | 5q31.2 | Consensussequence | Hs.256697.2 | N32864 | Hs.256697.2.A1 |
| | HG-U133B | | | | | protein 1 | | | | | |
| 36 | 214700_x_at | DKFZP434D193 | 64.77 | 6.39e−12 | 2.57e−09 | DKFZP434D193 protein | 2q23.3 | Consensussequence | Hs.225841.1 | AK000323.1 | Hs.225841.1.S1 |
| 37 | 236728_at | FLJ39485 | 63.39 | 1.77e−13 | 1.15e−10 | hypothetical protein FLJ39485 | 5q14.3 | Consensussequence | Hs.26330.0 | AW070437 | Hs.26330.0.A1 |
| 38 | 226656_at | CRTAP | 62.27 | 2.19e−12 | 1.02e−09 | cartilage associated protein | 3p22 | Consensussequence | Hs.155481.1 | AW024741 | Hs.155481.1_RC |
| 39 | 212737_at | GM2A | 62.11 | 3.22e−13 | 1.88e−10 | GM2 ganglioside activator | 5q31.3-q33.1 | Consensussequence | Hs.278242.3 | AL513583 | Hs.278242.3 |
| | | | | | | protein | | | | | |
| 40 | 201977_s_at | KIAA0141 | 60.74 | 4.37e−12 | 1.84e−09 | KIAA0141 gene product | 5q31.3 | Consensussequence | Hs.63510.0 | AI539425 | Hs.63510.0.A1 |
| 41 | 224767_at | | 59.87 | 1.63e−12 | 7.76e−10 | Homo sapiens mRNA; cDNA | | Consensussequence | Hs.80545.1 | AL137450.1 | Hs.80545.1_RC |
| | | | | | | DKFZp434J0428 (from clone | | | | | |
| | | | | | | DKFZp434J0428) | | | | | |
| 42 | 208130_s_at | TBXAS1 | 59.84 | 2.98e−13 | 1.86e−10 | thromboxane A synthase 1 | 7q34-q35 | Exemplarsequence | g13699839 | NM_030984.1 | g13699839 |
| | | | | | | (platelet, cytochrome P450, | | | | | |
| | | | | | | family 5, subfamily A) | | | | | |
| 43 | 238075_at | | 58.68 | 2.38e−11 | 7.67e−09 | ESTs, Weakly similar to A42442 | | Consensussequence | Hs.24529.0 | AA224205 | Hs.24529.0.A1 |
| | | | | | | integrin beta-1 chain, splice | | | | | |
| | | | | | | form beta-1-S - human | | | | | |
| | | | | | | (fragment) [H. sapiens] | | | | | |
| 44 | 201507_at | PFDN1 | 58.48 | 1.70e−11 | 5.92e−09 | prefoldin 1 | 5q31 | Exemplarsequence | Hs.132881.0 | NM_002622.2 | g12408673 |
| 45 | 226142_at | GLIPR1 | 56.83 | 2.30e−12 | 1.04e−09 | GLI pathogenesis-related 1 | 12q21.1 | Consensussequence | Hs.154762.2 | AV682252 | Hs.154762.2.A1 |
| | | | | | | (glioma) | | | | | |
| 46 | 211938_at | PRO1843 | 56.74 | 1.04e−11 | 3.84e−09 | hypothetical protein PRO1843 | 12q13.13 | Consensussequence | Hs.283330.1 | BF247371 | Hs.283330.1 |
| 47 | 201568_at | QP-C | 56.43 | 1.86e−11 | 6.35e−09 | low molecular mass ubiquinone- | 5q31.1 | Exemplarsequence | Hs.3709.0 | NM_014402.1 | g7657485 |
| | | | | | | binding protein (9.5 kD) | | | | | |
| 48 | 200608_s_at | RAD21 | 56.29 | 6.33e−11 | 1.85e−08 | RAD21 homolog (S. pombe) | 8q24 | Exemplarsequence | Hs.81848.0 | NM_006265.1 | g5453993 |
| 49 | 209523_at | TAF2 | 56.27 | 9.98e−11 | 2.65e−08 | TAF2 RNA polymerase II, TATA | 8q24.12 | Consensussequence | Hs.122752.0 | AK001618.1 | Hs.122752.0 |
| | | | | | | box binding protein (TBP)- | | | | | |
| | | | | | | associated factor, 150 kDa | | | | | |
| 50 | 241827_at | | 56.18 | 7.16e−11 | 1.99e−08 | Homo sapiens cDNA FLJ39372 | | Consensussequence | Hs.262036.0 | AL577866 | Hs.262036.0.S1 |
| | | | | | | fis, clone PEBLM2007832, | | | | | |
| | | | | | | moderately similar to ZINC | | | | | |
| | | | | | | FINGER PROTEIN 84. | | | | | |
| 51 | 206295_at | IL18 | 55.98 | 4.51e−12 | 1.85e−09 | interleukin 18 (interferon- | 11q22.2-q22.3 | Exemplarsequence | Hs.83077.0 | NM_001562.1 | g4504652 |
| | | | | | | gamma-inducing factor) | | | | | |
| 52 | 217751_at | LOC51064 | 55.62 | 1.10e−11 | 3.97e−09 | glutathione S-transferase | 7q34 | Exemplarsequence | Hs.279952.0 | NM_015917.1 | g7705703 |
| | | | | | | subunit 13 homolog | | | | | |
| 53 | 225090_at | HRD1 | 55.32 | 5.10e−11 | 1.51e−08 | HRD1 protein | 11q13 | Consensussequence | Hs.75859.1 | AA844682 | Hs.75859.1.A1 |
| 54 | 209303_at | NDUFS4 | 54.57 | 2.96e−11 | 9.22e−09 | NADH dehydrogenase | 5q11.1 | Exemplarsequence | Hs.10758.0 | BC005270.1 | g13528959 |
| | | | | | | (ubiquinone) Fe—S protein 4, | | | | | |
| | | | | | | 18 kDa (NADH-coenzyme Q | | | | | |
| | | | | | | reductase) | | | | | |
| 55 | 228855_at | | 54.42 | 2.36e−12 | 1.04e−09 | Homo sapiens cDNA: FLJ22816 | | Consensussequence | Hs.115920.0 | AI927964 | Hs.115920.0.A1 |
| | | | | | | fis, clone KAIA3155. | | | | | |
| 56 | 222984_at | PAIP2 | 54.39 | 2.76e−11 | 8.75e−09 | PABP-interacting protein 2 | 5q31.3 | Exemplarsequence | Hs.108548.0 | AF151052.1 | g7106825 |
| 57 | 218645_at | ZNF277 | 53.69 | 3.42e−11 | 1.03e−08 | zinc finger protein (C2H2 type) | 7q31.1 | Exemplarsequence | Hs.42636.0 | NM_021994.1 | g11496268 |
| | | | | | | 277 | | | | | |
| 58 | 202659_at | PSMB10 | 53.67 | 2.06e−11 | 6.77e−09 | proteasome (prosome, | 16q22.1 | Exemplarsequence | Hs.9661.0 | NM_002801.1 | g4506190 |
| | | | | | | macropain) subunit, beta type, | | | | | |
| | | | | | | 10 | | | | | |
| 59 | 244360_at | | 53.28 | 7.87e−12 | 3.09e−09 | ESTs | | Consensussequence | Hs.233425.0 | AW002273 | Hs.233425.0.A1 |
| 60 | 202105_at | IGBP1 | 53.19 | 6.48e−11 | 1.86e−08 | immunoglobulin (CD79A) | Xq13.1-q13.3 | Exemplarsequence | Hs.3631.0 | NM_001551.1 | g4557662 |
| | | | | | | binding protein 1 | | | | | |
| 61 | 201377_at | NICE-4 | 53.12 | 2.19e−10 | 5.01e−08 | NICE-4 protein | 1q21.3 | Exemplarsequence | Hs.8127.0 | NM_014847.1 | g7661941 |
| 62 | 218929_at | CARF | 52.81 | 2.52e−10 | 5.70e−08 | collaborates/cooperates with | 4q35.1 | Exemplarsequence | Hs.32922.0 | NM_017632.1 | g8923039 |
| | | | | | | ARF (alternate reading frame) | | | | | |
| | | | | | | protein | | | | | |
| 63 | 208021_s_at | RFC1 | 52.19 | 1.38e−10 | 3.42e−08 | replication factor C (activator 1) | 4p14-p13 | Exemplarsequence | g4506484 | NM_002913.1 | g4506484 |
| | | | | | | 1, 145 kDa | | | | | |
| 64 | 202633_at | TOPBP1 | 51.69 | 2.13e−10 | 4.94e−08 | topoisomerase (DNA) II binding | 3q22.1 | Exemplarsequence | Hs.91417.0 | NM_007027.1 | g5902137 |
| | | | | | | protein | | | | | |
| 65 | 210314_x_at | TNFSF13 | 51.46 | 1.90e−11 | 6.36e−09 | tumor necrosis factor (ligand) | 17p13.1 | Exemplarsequence | Hs.54673.1 | AF114013.1 | g7328557 |
| | | | | | | superfamily, member 13 | | | | | |
| 66 | 205366_s_at | HOXB6 | 51.24 | 1.17e−11 | 4.14e−09 | homeo box B6 | 17q21.3 | Exemplarsequence | Hs.98428.0 | NM_018952.1 | g9506792 |
| 67 | 2174g1_x_at | COX7C | 51.04 | 2.87e−10 | 6.19e−08 | cytochrome c oxidase subunit | 5q14 | Consensussequence | Hs.278494.0 | AF042165 | Hs.278494.0 |
| | | | | | | VIIc | | | | | |
| 68 | 209329_x_at | MGC2198 | 50.98 | 6.71e−11 | 1.89e−08 | hypothetical protein MGC2198 | 5q35.3 | Exemplarsequence | Hs.227152.1 | BC000587.1 | g12653618 |
| 69 | 224841_x_at | | 50.98 | 1.25e−10 | 3.22e−08 | Homo sapiens mRNA; cDNA | | Consensussequence | Hs.289721.2 | BF316352 | Hs.289721.2.S1 |
| | | | | | | DKFZp564D0164 (from clone | | | | | |
| | | | | | | DKFZp564D0164) | | | | | |
| 70 | 209022_at | STAG2 | 50.79 | 2.59e−10 | 5.79e−08 | stromal antigen 2 | Xq25 | Consensussequence | Hs.8217.1 | AK026678.1 | Hs.8217.1_RC |
| 71 | 233955_x_at | HSPC195 | 50.60 | 3.37e−11 | 1.03e−08 | hypothetical protein HSPC195 | 5q31.3 | Consensussequence | Hs.15093.1 | AK001782.1 | Hs.15093.1 |
| 72 | 201343_at | UBE2D2 | 50.57 | 7.73e−11 | 2.09e−08 | ubiquitin-conjugating enzyme | 5q31.3 | Consensussequence | Hs.108332.0 | NM_003339.1 | Hs.108332.0 |
| | | | | | | E2D 2 (UBC4/5 homolog, yeast) | | | | | |
| 73 | 239791_at | | 50.55 | 9.46e−12 | 3.64e−09 | Homo sapiens, clone | | Consensussequence | Hs.269918.1 | AI125255 | Hs.269918.1.A1 |
| | | | | | | MGC: 10077 IMAGE: 3896690, | | | | | |
| | | | | | | mRNA, complete cds | | | | | |
| 74 | 208697_s_at | EIF3S6 | 50.28 | 1.66e−10 | 4.06e−08 | eukaryotic translation initiation | 8q22-q23 | Exemplarsequence | Hs.106673.0 | BC000734.1 | g12653884 |
| | | | | | | factor 3, subunit 6 48 kDa | | | | | |
| 75 | 204112_s_at | HNMT | 50.23 | 9.76e−12 | 3.68e−09 | histamine N-methyltransferase | 2q21.3 | Exemplarsequence | Hs.81182.0 | NM_006895.1 | g5901969 |
| 76 | 224915_x_at | | 50.16 | 2.00e−10 | 4.70e−08 | Homo sapiens, clone | | Consensussequence | Hs.284158.1 | AV756131 | Hs.284158.1_RC |
| | | | | | | IMAGE: 5285034, mRNA | | | | | |
| 77 | 201696_at | SFRS4 | 49.66 | 7.51e−10 | 1.44e−07 | splicing factor, arginine/serine- | 1p35.2 | Exemplarsequence | Hs.76122.0 | NM_005626.1 | g5032088 |
| | | | | | | rich 4 | | | | | |
| 78 | 209619_at | CD74 | 49.48 | 7.68e−11 | 2.09e−08 | CD74 antigen (invariant | 5q32 | Exemplarsequence | Hs.84298.0 | K01144.1 | g188469 |
| | | | | | | polypeptide of major | | | | | |
| | | | | | | histocompatibility complex, | | | | | |
| | | | | | | class II antigen-associated) | | | | | |
| 79 | 208717_at | OXA1L | 49.39 | 1.08e−10 | 2.84e−08 | oxidase (cytochrome c) | 14q11.2 | Exemplarsequence | Hs.151134.0 | BC001669.1 | g12804516 |
| | | | | | | assembly 1-like | | | | | |
| 80 | 221593_s_at | VRP | 49.32 | 1.32e−10 | 3.36e−08 | vascular Rab-GAP/TBC- | 2q11.2 | Exemplarsequence | Hs.164170.1 | BC001663.1 | q12804504 |
| | | | | | | containing | | | | | |
| 81 | 212038_s_at | VDAC1 | 48.95 | 1.82e−10 | 4.40e−08 | voltaga-dependent anion | 5q31 | Consensusssequemce | Hs.149155.2 | AL515918 | Hs.149155.2.A1 |
| | | | | | | channel 1 | | | | | |
| 82 | 209259_s_at | CSPG6 | 48.87 | 8.12e−10 | 1.55e−07 | chondroitin sulfate proteoglycan | 10q25 | Exemplarsequence | Hs.24485.0 | AF020043.1 | g3089367 |
| | | | | | | 6 (bamacan) | | | | | |
| 83 | 208635_x_at | NACA | 48.58 | 2.94e−10 | 6.25e−08 | nascent-polypeptide-associatad | 12q23-q24.1 | Consensussequence | Hs.158164.1 | BF976260 | Hs.158164.1.S1 |
| | | | | | | complex alpha polypeptide | | | | | |
| 84 | 223015_at | eIF2a | 48.10 | 2.62e−10 | 5.79e−08 | eukaryolic translation initiation | 3q25.1 | Exemplarsequence | Hs.332404.0 | AF212241.1 | g13182760 |
| | | | | | | factor 2A eIF2a | | | | | |
| 85 | 224929_at | | 47.86 | 1.92e−10 | 4.56e−08 | Homo sapiens cDNA FLJ38577 | | Consensussequence | Hs.86347.1 | BG286973 | Hs.86347.1_RC |
| | | | | | | fis, clone HCHON2007650. | | | | | |
| 86 | 209408_at | KIF2C | 47.77 | 1.01e−09 | 1.89e−07 | kinesin family member 2C | 1p34.1 | Exemplarsequence | Hs.69360.1 | U63743.1 | g1695881 |
| 87 | 224741_x_at | | 47.22 | 4.18e−10 | 8.68e−08 | Homo sapiens mRNA; cDNA | | Consensussequence | Hs.289721.1 | BG329175 | Hs.289721.1.S1 |
| | | | | | | DKFZp564D0164 (from clone | | | | | |
| | | | | | | DKFZp564D0164) | | | | | |
| 88 | 216304_x_at | YME1L1 | 47.17 | 1.48e−09 | 2.67e−07 | YME1-like 1 (S. cerevisiae) | 10p14 | Consensussequence | Hs.206521.1 | AJ295618 | Hs.206521.1 |
| 89 | 214501_s_at | H2AFY | 47.15 | 2.75e−10 | 5.99e−08 | H2A histone family, member Y | 5q31.3-q32 | Consensussequence | Hs.75258.2 | AF044286.1 | Hs.75258.2 |
| 90 | 226227_x_at | | 47.02 | 6.76e−10 | 1.33e−07 | Homo sapiens, clone | | Consensussequence | Hs.284158.4 | BF185165 | Hs.284158.4 |
| | | | | | | IMAGE: 5285034, mRNA | | | | | |
| 91 | 226840_at | | 46.16 | 1.34e−10 | 3.36e−08 | Homo sapiens cDNA FLJ13771 | | Consensussequence | Hs.288934.0 | AW291297 | Hs.288934.0 |
| | | | | | | fis, clone PLACE4000270. | | | | | |
| 92 | 210758_at | PSIP1 | 45.75 | 3.08e−09 | 5.11e−07 | PC4 and SFRS1 interacting | 9p22.2 | Exemplarsequence | Hs.82110.0 | AF098482.1 | g4050033 |
| | | | | | | protein 1 | | | | | |
| 93 | 200735_x_at | NACA | 45.16 | 9.79e−10 | 1.84e−07 | nascent-polypeptide-associated | 12q23-q24.1 | Exemplarsequence | Hs.32916.0 | NM_005594.1 | g5031930 |
| | | | | | | complex alpha polypeptide | | | | | |
| 94 | 210844_x_at | CTNNA1 | 45.11 | 5.70e−10 | 1.15e−07 | catenin (cadherin-associated | 5q31 | Exemplarsequence | Hs.178452.1 | D14705.1 | g415305 |
| | | | | | | protein), alpha 1, 102 kDa | | | | | |
| 95 | 219590_x_at | CGI-30 | 44.95 | 5.27e−10 | 1.07e−07 | CGI-30 protein | 1p21.1 | Exemplarsequence | Hs.19978.0 | NM_015958.1 | g7706271 |
| 96 | 218013_x_at | DCTN4 | 44.82 | 6.71e−10 | 1.33e−07 | dynactin 4 (p62) | 5q31-q32 | Exemplarsequence | Hs.180952.0 | NM_016221.1 | g7705892 |
| 97 | 218735_s_at | AF020591 | 44.59 | 2.81e−09 | 4.75e−07 | zinc finger protein | 19q13.43 | Consensussequence | Hs.142634.0 | AA349848 | Hs.142634.0.S1 |
| 98 | 212018_s_at | DKFZP564M182 | 44.11 | 7.48e−10 | 1.44e−07 | DKFZP564M182 protein | 16p13.13 | Consensussequence | Hs.85963.0 | AK025446.1 | Hs.85963.0 |
| 99 | 218619_s_at | SUV39H1 | 44.08 | 2.41e−09 | 4.12e.07 | suppressor of variegation 3-9 | Xp11.23 | Exemplarsequence | Hs.37936.0 | NM_003173.1 | g4507320 |
| | | | | | | homolog 1 (Drosophila) | | | | | |
| 100 | 201352_at | YME1L1 | 43.71 | 3.63e−09 | 5.81e−07 | YME1-like 1 (S. cerevisiae) | 10p14 | Exemplarsequence | Hs.206521.0 | NM_014263.1 | g7657688 |

| | Sequence | | | | |
|---|---|---|---|---|---|
| # | Source | Unigene_Accession | Cluster_Type | LocusLink | Full_Length_Reference_Seq |
| 1 | Genbank | Hs.8715 | fulllength | 84273 | NM_032313; hypothetical protein MGC3232 |
| 2 | RefSeq | Hs.297875 | fulllength | 64374 | NM_022464; endoplasmic reticulum chaperone SIL1, homolog of yeast |
| 3 | RefSeq | Hs.8170 | fulllength | 10412 | NM_014886; TGF beta-inducible nuclear protein 1 |
| 4 | RefSeq | Hs.63510 | fulllength | 9812 | NM_014773; KIAA0141 gene product |
| 5 | GenBank | Hs.446502 | est | | |
| 6 | GenBank | | | | |
| 7 | GenBank | Hs.122669 | fulllength | 23107 | NM_015084; mitochondrial ribosomal protein S27 |
| 8 | RefSeq | Hs.77324 | fulllength | 2107 | NM_004730; eukaryotic translation termination factor 1 |
| 9 | GenBank | Hs.379754 | | | |
| 10 | GenBank | Hs.396644 | fulllength | 51247 | NM_016480; polyadenylate-binding protein-interacting protein 2 |
| 11 | GenBank | Hs.256697 | fulllength | 3094 | NM_005340; histidine triad nucleotide binding protein 1 |
| 12 | GenBank | Hs.154574 | est | | |
| 13 | GenBank | Hs.211539 | fulllength | 1968 | NM_001415; eukaryotic translation initiation factor 2, subunit 3 gamma, |
| | | | | | 52 kDa |
| 14 | GenBank | Hs.301226 | fulllength | | NM_003732; eukaryotic translation initiation factor 4E binding protein 3 |
| 15 | GenBank | Hs.74267 | fulllength | 6138 | NM_002948; ribosomal protein L15 |
| 16 | RefSeq | Hs.13572 | fulllength | 819 | NM_001745; calcium modulating ligand |
| 17 | GenBank | Hs.29444 | fulllength | 85027 | NM_032947; putative small membrane protein NID67 |
| 18 | GenBank | Hs.406051 | fulllength | 54833 | NM_017685; hypothetical protein FLJ20139 |
| 19 | GenBank | Hs.256697 | fulllength | 3094 | NM_005340; histidine triad nucleotide binding protein 1 |
| 20 | RefSeq | Hs.256697 | fulllength | 3094 | NM_005340; histidine triad nucleotide binding protein 1 |
| 21 | GenBank | Hs.431584 | fulllength | 6208 | NM_005617; ribosomal protein S14 |
| 22 | GenBank | Hs.235422 | fulllength | 9045 | NM_003973; ribosomal protein L14 |
| 23 | RefSeq | Hs.283437 | fulllength | 56951 | NM_020199; HTGN29 protein |
| 24 | GenBank | Hs.106469 | fulllength | 6832 | NM_003171; suppressor of var1, 3-like 1 |
| 25 | GenBank | Hs.289082 | fulllength | 2760 | NM_000405; GM2 ganglioside activator protein precursor |
| 26 | RefSeq | Hs.108332 | fulllength | 7322 | NM_003339; ubiquitin-conjugating enzyme E2D 2 (UBC4/5 homolog, yeast) |
| 27 | GenBank | Hs.430075 | fulllength | 1350 | NM_001867; cytochrome c oxidase subunit VIIc precursor |
| 28 | RefSeq | Hs.240457 | fulllength | 5883 | NM_004584; RAD9 homolog |
| 29 | GenBank | Hs.441133 | fulllength | 51611 | NM_015958; CGI-30 protein |
| 30 | RefSeq | Hs.35086 | fulllength | 7398 | NM_003368; ubiquitin specific protease 1 |
| 31 | GenBank | Hs.91773 | fulllength | 5515 | NM_002715; protein phosphatase 2 (formerly 2A), catalytic subunit, alpha |
| | | | | | isoform |
| 32 | GenBank | Hs.183096 | fulllength | | |
| 33 | RefSeq | Hs.257111 | fulllength | 78991 | NM_024028; hypothetical protein MGC3265 |
| 34 | RefSeq | Hs.79322 | fulllength | 5859 | NM_005051; glutaminyl-tRNA synthetase |
| 35 | GenBank | Hs.256697 | fulllength | 3094 | NM_005340; histidine triad nucleotide binding protein 1 |
| 36 | GenBank | Hs.225841 | | 26109 | |
| 37 | GenBank | Hs.391318 | | 285603 | NM_175920; hypothetical protein FLJ39485 |
| 38 | GenBank | Hs.155481 | fulllength | 10491 | NM_006371; cartilage associated protein |
| 39 | GenBank | Hs.289082 | fulllength | 2760 | NM_000405; GM2 ganglioside activator protein precursor |
| 40 | GenBank | Hs.63510 | fulllength | 9812 | NM_014773; KIAA0141 gene product |
| 41 | GenBank | Hs.80545 | | | |
| 42 | RefSeq | Hs.2001 | fulllength | 6916 | NM_001061; thromboxane A synthase 1 (platelet, cytochrome P450, family |
| | | | | | 5, subfamily A) isoform TXS-I NM_030984; thromboxane A synthase 1 |
| | | | | | (platelet, cytochrome P450, family 5, subfamily A) isoform TXS-II |
| 43 | GenBank | Hs.24529 | est | | |
| 44 | RefSeq | Hs.132881 | fulllength | 5201 | NM_002622; prefoldin 1 |
| 45 | GenBank | Hs.64639 | fulllength | 11010 | NM_006851; glioma pathogenesis-related protein |
| 46 | GenBank | Hs.283330 | fulllength | 55378 | NM_018507; hypothetical protein PRO1843 |
| 47 | RefSeq | Hs.3709 | fulllength | 27089 | NM_014402; low molecular mass ubiquinone-binding protein |
| 48 | RefSeq | Hs.81848 | fulllength | 5885 | NM_006265; RAD21 homolog |
| 49 | GenBank | Hs.122752 | fulllength | 6873 | NM_003184; TBP-associated factor 2 |
| 50 | GenBank | Hs.262036 | | | |
| 51 | RefSeq | Hs.83077 | fulllength | 3606 | NM_001562; interleukin 18 proprotein |
| 52 | RefSeq | Hs.279952 | fulllength | 51064 | NM_015917; glutathione S-transferase subunit 13 homolog |
| 53 | GenBank | Hs.334819 | fulllength | 84447 | NM_032431; HRD1 protein isoform a NM_172230; HRD1 protein isoform b |
| 54 | GenBank | Hs.10758 | fulllength | 4724 | NM_002495; NADH dehydrogenase (ubiquinone) Fe—S protein 4, 18 kDa |
| | | | | | (NADH-coenzyme Q reductase) |
| 55 | GenBank | Hs.115920 | | | |
| 56 | GenBank | Hs.396644 | fulllength | 51247 | NM_016480; polyadenylate-binding protein-interacting protein 2 |
| 57 | RefSeq | Hs.42636 | fulllength | 11179 | NM_021994; zinc finger protein (C2H2 type) 277 |
| 58 | RefSeq | Hs.9661 | fulllength | 5699 | NM_002801; proteasome beta 10 subunit proprotein |
| 59 | GenBank | Hs.233425 | est | | |
| 60 | RefSeq | Hs.3631 | fulllength | 3476 | NM_001551; immunoglobulin (CD79A) binding protein 1 |
| 61 | RefSeq | Hs.8127 | fulllength | 9898 | NM_014847; NICE-4 protein |
| 62 | RefSeq | Hs.32922 | fulllength | 55602 | NM_017632; collaborates/cooperates with ARF (alternate reading frame) |
| | | | | | protein |
| 63 | RefSeq | Hs.166563 | fulllength | 5981 | NM_002913; replication factor C large subunit NM_006081; |
| 64 | RefSeq | Hs.91417 | fulllength | 11073 | NM_007027; topoisomerase (DNA) II binding protein |
| 65 | GenBank | Hs.54673 | fulllength | 8741 | NM_003808; tumor necrosis factor ligand superfamily, member 13 isoform |
| | | | | | alpha precursor NM_172087; tumor necrosis factor ligand superfamily, |
| | | | | | member 13 isoform beta NM_172088; tumor necrosis factor ligand |
| | | | | | superfamily, member 13 isoform gamma NM_172089; tumor necrosis factor |
| | | | | | ligand superfamily, member 13 isoform delta |
| 66 | RefSeq | Hs.98428 | fulllength | 3216 | NM_018952; homeo box B6 isoform 1 NM_156036; homeo box B6 isoform |
| | | | | | 2 NM_156037; homeo box B6 isoform 1 |
| 67 | GenBank | Hs.430075 | fulllength | 1350 | NM_001867; cytochrome c oxidase subunit VlIc precursor |
| 68 | GenBank | Hs.433267 | fulllength | 192286 | NM_138820; hypothetical protein MGC2198 |
| 69 | GenBank | Hs.289721 | | | |
| 70 | GenBank | Hs.8217 | fulllength | 10735 | NM_006603; stromal antigen 2 |
| 71 | GenBank | Hs.15093 | fulllength | 51523 | NM_016463; hypothetical protein HSPC195 |
| 72 | GenBank | Hs.108332 | fulllength | 7322 | NM_003339; ubiquitin-conjugating enzyme E2D 2 (UBC4/5 homolog, yeast) |
| 73 | GenBank | Hs.183096 | fulllength | | |
| 74 | GenBank | Hs.106673 | fulllength | 3646 | NM_001568; murine mammary tumor integration site 6 (oncogene |
| | | | | | homolog) |
| 75 | RefSeq | Hs.81182 | fulllength | 3176 | NM_006895; histamine N-methyltransferase |
| 76 | GenBank | Hs.356766 | | | |
| 77 | RefSeq | Hs.76122 | fulllength | 6429 | NM_005626; splicing factor, arginine/serine-rich 4 |
| 78 | GenBank | Hs.84298 | fulllength | 972 | NM_004355; invariant gamma chain |
| 79 | GenBank | Hs.151134 | fulllength | 5018 | NM_005015; oxidase (cytochrome c) assembly 1-like |
| 80 | GenBank | Hs.164170 | fulllength | 11138 | NM_007063; TBC1 domain family, member 8 (with GRAM domain) |
| 81 | GenBank | Hs.149155 | fulllength | 7416 | NM_003374; voltage-dependent anion channel 1 |
| 82 | GenBank | Hs.24485 | fulllength | 9126 | NM_005445; chondroitin sulfate proteoglycan 6 (bamacan) |
| 83 | GenBank | Hs.32916 | fulllength | 4666 | NM_005594; nascent-polypeptide-associated complex alpha polypeptide |
| 84 | GenBank | Hs.332404 | fulllength | 83939 | NM_032025; CDA02 protein |
| 85 | GenBank | Hs.379754 | | | |
| 86 | GenBank | Hs.69360 | fulllength | 11004 | NM_006845; kinesin family member 2C |
| 87 | GenBank | Hs.289721 | | | |
| 88 | GenBank | Hs.206521 | fulllength | 10730 | NM_014263; YME1-like 1 isoform 3 NM_139312; YME1-like 1 isoform 1 |
| | | | | | NM_139313; YME1-like 1 isoform 2 |
| 89 | GenBank | Hs.75258 | fulllength | 9555 | NM_004893; H2A histone family, member Y isoform 2 NM_138609; H2A |
| | | | | | histone family, member Y isoform 1 NM_138610; H2A histone family, |
| | | | | | member Y isoform 3 |
| 90 | GenBank | Hs.356766 | | | |
| 91 | GenBank | Hs.288934 | | | |
| 92 | GenBank | Hs.351305 | fulllength | 9090 | |
| 93 | RefSeq | Hs.32916 | fulllength | 4666 | NM_005594; nascent-polypeptide-aasociated complex alpha polypeptide |
| 94 | GenBank | Hs.177556 | fulllength | 1495 | NM_001903; catenin (cadherin-associated protein), alpha 1, 102 kDa |
| 95 | RefSeq | Hs.441133 | fulllength | 51611 | NM_015958; CGI-30 protein |
| 96 | RefSeq | Hs.328865 | fulllength | 51164 | NM_016221; dynactin 4 (p62) |
| 97 | GenBank | Hs.142634 | fulllength | 27300 | NM_014480; zinc finger protein AF020591 |
| 98 | GenBank | Hs.85963 | fulllength | 26156 | |
| 99 | RefSeq | Hs.37936 | fulllength | 6839 | NM_003173; suppressor of variegation 3-9 homolog 1 |
| 100 | RefSeq | Hs.206521 | fulllength | 10730 | NM_014263; YME1-like 1 isoform 3 NM_139312; YME1-like 1 isoform 1 |
| | | | | | NM_139313; YME1-like 1 isoform 2 |

TABLE 38

| | | | | | | | | | | | Sequence |
|---|---|---|---|---|---|---|---|---|---|---|---|
| | | HUGO | | | | | | Map | Sequence | | Derived |
| Number | affy id | name | fc | p | q | stn | t | Location | Type | Transcript ID | From |
| 74 | 231982_at | | 4.69 | 8.4E−23 | 1.89E−19 | 0.81 | 11.41 | | Consensus | Hs.130714.0 | AF161441.1 |
| | | | | | | | | | sequence | | |
| 62 | 209160_at | AKR1C3 | 3.98 | 4.56E−24 | 2.53E−20 | 0.81 | 11.58 | 10p15-p14 | Exemplar | Hs.78183.0 | AB018580.1 |
| | | | | | | | | | sequence | | |
| 142 | 235142_at | MGC17919 | 3.79 | 1.34E−19 | 9.66E−17 | 0.71 | 10.13 | 1p34.3 | Consensus | Hs.129837.0 | AW006067 |
| | | | | | | | | | sequence | | |
| 124 | 230460_at | | 3.61 | 2.7E−20 | 2.99E−17 | 0.72 | 10.31 | | Consensus | Hs.79844.1 | AI609804 |
| | | | | | | | | | sequence | | |
| 190 | 213258_at | | 3.38 | 4.31E−18 | 1.56E−15 | 0.68 | 9.68 | | Consensus | Hs.288582.0 | BF511231 |
| | | | | | | | | | sequence | | |
| 300 | 226342_at | | 3.38 | 2E−16 | 4.06E−14 | 0.63 | 8.97 | | Consensus | Hs.238443.0 | AW593244 |
| | | | | | | | | | sequence | | |
| 269 | 210664_s_at | TFPI | 3.33 | 4.86E−16 | 8.4E−14 | 0.66 | 9.14 | 2q31-q32.1 | Exemplar | Hs.170279.1 | AF021834.1 |
| | | | | | | | | | sequence | | |
| 258 | 230894_s_at | | 3.27 | 1.73E−15 | 2.41E−13 | 0.69 | 9.19 | | Consensus | Hs.42640.1 | BE672557 |
| | | | | | | | | | sequence | | |
| 140 | 225240_s_at | | 3.24 | 9.79E−19 | 4.62E−16 | 0.73 | 10.15 | | Consensus | Hs.42179.0 | BF435123 |
| | | | | | | | | | sequence | | |
| 161 | 202933_s_at | YES1 | 3.04 | 4.16E−19 | 2.38E−16 | 0.7 | 9.97 | 18p11.31-p11.21 | Exemplar | Hs.194148.0 | NM_005433.1 |
| | | | | | | | | | sequence | | |
| 218 | 226134_s_at | | 3.02 | 1.38E−16 | 3.06E−14 | 0.69 | 9.42 | | Consensus | Hs.42640.0 | AI978754 |
| | | | | | | | | | sequence | | |
| 139 | 202932_at | YES1 | 2.99 | 8.22E−20 | 6.83E−17 | 0.71 | 10.15 | 18p11.31-p11.21 | Exemplar | Hs.194148.0 | NM_005433.1 |
| | | | | | | | | | sequence | | |
| 242 | 227860_at | CPXM | 2.95 | 3.39E−17 | 8.81E−15 | 0.66 | 9.31 | 20p12.3-p13 | Consensus | Hs.177536.0 | NM_019609.1 |
| | | | | | | | | | sequence | | |
| 163 | 238498_at | | 2.86 | 2.75E−18 | 1.12E−15 | 0.72 | 9.95 | | Consensus | Hs.46922.0 | AA583038 |
| | | | | | | | | | sequence | | |
| 138 | 211071_s_at | AF1Q | 2.81 | 9.98E−20 | 7.9E−17 | 0.71 | 10.16 | 1q21 | Exemplar | g13623686 | BC006471.1 |
| | | | | | | | | | sequence | | |
| 180 | 228046_at | LOC152485 | 2.4 | 1.48E−18 | 6.45E−16 | 0.69 | 9.79 | 4q31.1 | Consensus | Hs.11325.0 | AA741243 |
| | | | | | | | | | sequence | | |
| 196 | 204453_at | ZNF84 | 2.32 | 1.13E−17 | 3.43E−15 | 0.69 | 9.65 | 12q24.33 | Exemplar | Hs.9450.0 | NM_003428.1 |
| | | | | | | | | | sequence | | |
| 127 | 226043_at | AGS3 | 2.22 | 3.82E−18 | 1.44E−15 | 0.76 | 10.27 | 9q34.3 | Consensus | Hs.239370.0 | AI242661 |
| | | | | | | | | | sequence | | |
| 131 | 226157_at | | 2.22 | 2.55E−19 | 1.69E−16 | 0.73 | 10.24 | | Consensus | Hs.19131.2 | AI569747 |
| | | | | | | | | | sequence | | |
| 212 | 242028_at | FLJ38281 | 2.17 | 1.29E−17 | 3.83E−15 | 0.67 | 9.48 | 19p13.13 | Consensus | Hs.293223.0 | AI800025 |
| | | | | | | | | | sequence | | |
| 195 | 227934_at | | 2.09 | 4.56E−18 | 1.59E−15 | 0.68 | 9.66 | | Consensus | Hs.24252.0 | AI789559 |
| | | | | | | | | | sequence | | |
| 271 | 204663_at | ME3 | 2.06 | 2.27E−16 | 4.48E−14 | 0.65 | 9.14 | 11cen-q22.3 | Exemplar | Hs.2838.0 | NM_006680.1 |
| | | | | | | | | | sequence | | |
| 296 | 223714_at | ZNF258 | 2.06 | 2E−15 | 2.7E−13 | 0.66 | 8.99 | 19q13.43 | Exemplar | Hs.161751.0 | BC001438.1 |
| | | | | | | | | | sequence | | |
| 151 | 209490_s_at | PPT2 | 2.04 | 6.73E−19 | 2.89E−16 | 0.72 | 10.07 | 6p21.3 | Exemplar | Hs.81737.0 | AF020543.1 |
| | | | | | | | | | sequence | | |
| 235 | 202732_at | PKIG | 2.04 | 3.65E−14 | 2.97E−12 | 0.77 | 9.36 | 20q12-q13.1 | Exemplar | Hs.3407.0 | NM_007066.1 |
| | | | | | | | | | sequence | | |
| 63 | 202365_at | MGC5139 | 1.57 | 2.9E−22 | 2.9E−19 | 0.84 | 11.57 | 12q24.31 | Exemplar | Hs.127610.0 | BC004815.1 |
| | | | | | | | | | sequence | | |
| 206 | 228402_at | MGC15435 | 1.88 | 2.19E−17 | 5.86E−15 | 0.68 | 9.51 | 5q13.2 | Consensus | Hs.152060.0 | AI679968 |
| | | | | | | | | | sequence | | |
| 186 | 37590_g_at | | 1.83 | 3.82E−16 | 7.05E−14 | 0.75 | 9.75 | | Consensus | 4876007 | AL109698 |
| | | | | | | | | | sequence | | |
| 150 | 225314_at | MGC45416 | 1.82 | 3.15E−17 | 8.31E−15 | 0.78 | 10.08 | 4p11 | Consensus | Hs.95835.0 | BG291649 |
| | | | | | | | | | sequence | | |
| 225 | 213779_at | LOC129080 | 1.82 | 8.76E−17 | 1.6E−14 | 0.68 | 9.41 | 22q12.1 | Consensus | Hs.289106.0 | AL031186 |
| | | | | | | | | | sequence | | |
| 285 | 218966_at | MYO5C | 1.8 | 1.41E−16 | 3.09E−14 | 0.64 | 9.06 | 15q21 | Exemplar | Hs.111782.0 | NM_018728.1 |
| | | | | | | | | | sequence | | |
| 209 | 213666_at | 38236 | 1.79 | 6.49E−17 | 1.57E−14 | 0.69 | 9.5 | Xq24 | Consensus | Hs.90998.1 | AK026589.1 |
| | | | | | | | | | sequence | | |
| 208 | 223162_s_at | LCHN | 1.76 | 1.48E−16 | 3.17E−14 | 0.7 | 9.5 | 7q34 | Exemplar | Hs.12461.0 | AF116707.1 |
| | | | | | | | | | sequence | | |
| 129 | 224841_x_at | | 1.5 | 1.51E−15 | 2.12E−13 | 0.86 | 10.25 | | Consensus | Hs.289721.2 | BF316352 |
| | | | | | | | | | sequence | | |
| 154 | 224741_x_at | | 1.5 | 4.27E−15 | 5.03E−13 | 0.85 | 10.05 | | Consensus | Hs.289721.1 | BG329175 |
| | | | | | | | | | sequence | | |

| | | | | | | Full |
|---|---|---|---|---|---|---|
| | | | | | | Length |
| | Sequence | Sequence | Unigene | Cluster | Locus | Reference |
| Number | ID | Source | Accession | Type | Link | Seq |
| 74 | Hs.130714.0 | GenBank | Hs.130714 | | | |
| 62 | g6624210 | GenBank | Hs.78183 | fulllength | 8644 | NM_003739; aldo-keto reductase family 1, member C3 |
| 142 | Hs.129837.0_RC | GenBank | Hs.129837 | fulllength | 127557 | NM_144621; hypothetical protein MGC17919 |
| 124 | Hs.79844.1.A1 | GenBank | Hs.433736 | | | |
| 190 | Hs.288582.0_RC | GenBank | Hs.87980 | est | | |
| 300 | Hs.236443.0.S1 | GenBank | Hs.236443 | | | |
| 269 | g4103170 | GenBank | Hs.170279 | fulllength | 7035 | NM_006287; tissue factor pathway inhibitor |
| | | | | | | (lipoprotein-associated coagulation inhibitor) |
| 258 | Hs.42640.1.A1 | GenBank | Hs.173179 | | | |
| 140 | Hs.42179.0.A1 | GenBank | Hs.173179 | | | |
| 161 | g4885660 | RefSeq | Hs.194148 | fulllength | 7525 | NM_005433; viral oncogene yes-1 homolog 1 |
| 218 | Hs.42640.0.A1 | GenBank | Hs.173179 | | | |
| 139 | g4885660 | RefSeq | Hs.194148 | fulllength | 7525 | NM_005433; viral oncogene yes-1 homolog 1 |
| 242 | Hs.177536.0 | GenBank | Hs.177536 | fulllength | 56265 | NM_019609; metallocarboxypeptidase CPX-1 precursor |
| 163 | Hs.46922.0.A1 | GenBank | Hs.349207 | | | |
| 138 | g13623686 | GenBank | Hs.75823 | fulllength | 10962 | NM_006818; AF1Q protein |
| 180 | Hs.11325.0.A1 | GenBank | Hs.351270 | | 152485 | NM_178835; hypothetical protein LOC152485 |
| 196 | g4508036 | RefSeq | Hs.9450 | fulllength | 7637 | NM_003428; zinc finger protein 84 (HPF2) |
| 127 | Hs.239370.0.S1 | GenBank | Hs.239370 | fulllength | 26086 | NM_015597; activator of G-protein signaling 3 |
| 131 | Hs.19131.2.A1 | GenBank | Hs.398090 | | | |
| 212 | Hs.293223.0.A1 | GenBank | Hs.293223 | fulllength | 163051 | NM_152601; hypothetical protein FLJ38281 |
| 195 | Hs.24252.0_RC | GenBank | Hs.24252 | est | | |
| 271 | g5729919 | RefSeq | Hs.376590 | fulllength | 10873 | NM_006680; malic enzyme 3, NADP(+)-dependent, |
| | | | | | | mitochondrial |
| 296 | g12655164 | GenBank | Hs.161751 | fulllength | 10172 | NM_005773; zinc finger protein 256 |
| 151 | g2501960 | GenBank | Hs.81737 | fulllength | 9374 | NM_005155; palmitoyl-protein thioesterase 2 isoform a precursor |
| | | | | | | NM_138717; palmitoyl-protein thioesterase 2 isoform b precursor |
| | | | | | | NM_138934; palmitoyl-protein thioesterase 2 isoform c precursor |
| 235 | g5902019 | RefSeq | Hs.3407 | fulllength | 11142 | NM_007068; protein kinase (cAMP-dependent, catalytic) inhibitor gamma |
| 63 | g13435956 | GenBank | Hs.127610 | fulllength | 84747 | NM_032661; hypothetical protein MGC5139 |
| 206 | Hs.152060.0_RC | GenBank | Hs.152060 | fulllength | 84327 | NM_032367; zinc finger, BED domain containing 3 |
| 186 | 4876007 | GenBank | Hs.323053 | | | |
| 150 | Hs.95835.0.A1 | GenBank | Hs.95835 | fulllength | 132299 | NM_152398; hypothetical protein MGC45418 |
| 225 | Hs.289106.0.S1 | GenBank | Hs.289106 | fulllength | 129080 | NM_133455; emilin and multimerin-domain containing protein 1 |
| 285 | g9055283 | RefSeq | Hs.111782 | fulllength | 55930 | NM_018728; myosin VC |
| 209 | Hs.90998.1.S1 | GenBank | Hs.90998 | fulllength | 23157 | NM_015129; septin 6 isoform B NM_145799; |
| | | | | | | septin 6 isoform A NM_145800; |
| | | | | | | septin 6 isoform A NM_145802; |
| | | | | | | septin 6 isoform D |
| 208 | g7959912 | GenBank | Hs.233044 | fulllength | 57189 | |
| 129 | Hs.289721.2.S1 | GenBank | Hs.289721 | | | |
| 154 | Hs.289721.1.S1 | GenBank | Hs.289721 | | | |

TABLE 39

| | | | | | | | | Map |
|---|---|---|---|---|---|---|---|---|
| Number | affy id | HUGO name | fc | p | q | stn | t | Location |
| 239 | 211135_x_at | LILRB3 | −12.15 | 6.41E−12 | 2.38E−10 | −1.18 | −9.35 | 19q13.4 |
| 278 | 210225_x_at | LILRB3 | −9.8 | 1.21E−11 | 3.92E−10 | −1.11 | −9.1 | 19q13.4 |
| 132 | 210784_x_at | LILRB3 | −9.47 | 3.39E−13 | 1.95E−11 | −1.22 | −10.23 | 19q13.4 |
| 275 | 215646_s_at | CSPG2 | −9.47 | 1.07E−11 | 3.55E−10 | −1.09 | −9.12 | 5q14.3 |
| 96 | 205936_s_at | HK3 | −8.7 | 4.72E−14 | 3.75E−12 | −1.3 | −10.88 | 5q35.2 |
| 89 | 226841_at | LOC219972 | −7.61 | 1.81E−14 | 1.63E−12 | −1.27 | −11.11 | 11q12.1 |
| 264 | 205789_at | CD1D | −7.35 | 8.04E−12 | 2.83E−10 | −1.05 | −9.16 | 1q22-q23 |
| 210 | 210992_x_at | FCGR2A | −7.21 | 3.54E−12 | 1.49E−10 | −1.15 | −9.49 | 1q23 |
| 83 | 220266_s_at | KLF4 | −6.87 | 1.38E−14 | 1.3E−12 | −1.29 | −11.21 | 9q31 |
| 299 | 211133_x_at | LILRB3 | −6.55 | 1.85E−11 | 5.67E−10 | −1.1 | −8.98 | 19q13.4 |
| 19 | 226818_at | LOC219972 | −6.45 | 9.4E−18 | 2.89E−15 | −1.45 | −13.49 | 11q12.1 |
| 35 | 202295_s_at | CTSH | −6.45 | 2.21E−16 | 4.43E−14 | −1.41 | −12.56 | 15q24-q25 |
| 211 | 206934_at | SIRP81 | −6.42 | 3.18E−12 | 1.38E−10 | −1.12 | −9.49 | 20p13 |
| 259 | 219788_at | PILR | −6.11 | 1.09E−11 | 3.59E−10 | −1.17 | −9.18 | 7q22 |
| 21 | 201422_at | FI30 | −5.99 | 4.24E−18 | 1.56E−15 | −1.34 | −13.36 | 19p13.1 |
| 54 | 222218_s_at | PILR | −5.97 | 4.02E−15 | 4.84E−13 | −1.42 | −11.75 | 7q22 |
| 76 | 222670_s_at | MAFB | −5.73 | 3.58E−15 | 4.4E−13 | −1.2 | −11.37 | 20q11.2-q13.1 |
| 69 | 211429_s_at | SERPINA1 | −5.7 | 4.62E−15 | 5.39E−13 | −1.29 | −11.51 | 14q32.1 |
| 267 | 212192_at | LOC115207 | −5.66 | 6E−12 | 2.26E−10 | −0.99 | −9.14 | 13q21.33 |
| 100 | 209696_at | FBP1 | −5.57 | 5.12E−14 | 4.01E−12 | −1.26 | −10.82 | 9q22.3 |
| 202 | 210895_s_at | CD86 | −5.57 | 1.92E−12 | 8.88E−11 | −1.1 | −9.6 | 3q21 |
| 44 | 206278_at | PTAFR | −5.58 | 6.23E−16 | 1.01E−13 | −1.34 | −12.14 | 1p35-p34.3 |
| 224 | 213566_at | RNASE6 | −5.51 | 2.42E−12 | 1.09E−10 | −1.02 | −9.41 | 14q11.1 |
| 31 | 221731_x_at | CSPG2 | −5.42 | 1.53E−17 | 4.46E−15 | −1.24 | −12.7 | 5q14.3 |
| 152 | 202878_s_at | C1QR1 | −5.39 | 4.95E−13 | 2.67E−11 | −1.17 | −10.07 | 20p11.21 |
| 250 | 204961_s_at | NCF1 | −5.37 | 3.81E−12 | 1.57E−10 | −0.98 | −9.24 | 7q11.23 |
| 25 | 204820_s_at | CSPG2 | −5.15 | 5.76E−18 | 1.91E−15 | −1.25 | −12.94 | 5q14.3 |
| 48 | 218559_s_at | MAFB | −5.15 | 1.24E−16 | 2.83E−14 | −1.15 | −11.97 | 20q11.2-q13.1 |
| 18 | 218217_at | RISC | −5.13 | 5.4E−18 | 1.83E−15 | −1.42 | −13.55 | 17q23.1 |
| 101 | 206380_s_at | PFC | −5.13 | 4.28E−14 | 3.46E−12 | −1.23 | −10.81 | Xp11.3-p11.23 |
| 256 | 220088_at | C5R1 | −5.06 | 5.3E−12 | 2.04E−10 | −1.01 | −9.21 | 19q13.3-q13.4 |
| 11 | 200782_at | ANXA5 | −5 | 3.18E−19 | 2.03E−16 | −1.41 | −14.17 | 4q28-q32 |
| 70 | 203767_s_at | STS | −4.82 | 4.64E−15 | 5.39E−13 | −1.29 | −11.51 | Xp22.32 |
| 34 | 222496_s_at | FLJ20273 | −4.75 | 6.33E−17 | 1.57E−14 | −1.29 | −12.57 | 4p13-p12 |
| 228 | 205237_at | FCN1 | −4.73 | 2.29E−12 | 1.04E−10 | −1 | −9.39 | 9p34 |
| 284 | 205685_at | CD86 | −4.71 | 8.24E−12 | 2.88E−10 | −1 | −9.07 | 3p21 |
| 137 | 220066_at | CARD15 | −4.54 | 2.36E−13 | 1.46E−11 | −1.11 | −10.16 | 16p12-q21 |
| 42 | 209949_at | NCF2 | −4.48 | 1.99E−16 | 4.06E−14 | −1.24 | −12.17 | 1q25 |
| 12 | 204232_at | FCER1G | −4.33 | 2.66E−18 | 1.11E−15 | −1.51 | −14 | 1q23 |
| 4 | 208438_s_at | FGR | −1.31 | 2.39E−20 | 2.99E−17 | −1.54 | −15.23 | 1p36.2-p36.1 |
| 294 | 234985_at | LOC143458 | −4.3 | 1.08E−11 | 3.56E−10 | −1.01 | −9.02 | 11p13 |
| 167 | 208983_a_at | PECAM1 | −4.28 | 3.21E−13 | 1.87E−11 | −1.03 | −9.93 | 17q23 |
| 46 | 219505_at | CECR1 | −4.18 | 5.08E−16 | 8.7E−14 | −1.26 | −12 | 22q11.2 |
| 130 | 211661_x_at | PTAFR | −4.01 | 2.08E−13 | 1.33E−11 | −1.14 | −10.24 | 1p35-p34.3 |
| 72 | 211101_x_at | LILRA2 | −3.99 | 3.83E−15 | 4.64E−13 | −1.24 | −11.46 | 19q13.4 |
| 79 | 204502_at | SAMHD1 | −3.98 | 3.39E−15 | 4.24E−13 | −1.15 | −11.26 | 20pter-q12 |
| 121 | 204081_at | NRGN | −3.95 | 7.5E−14 | 5.55E−12 | −1.06 | −10.33 | 11q24 |
| 94 | 224356_x_at | MSA4A6A | −3.92 | 3.05E−15 | 3.92E−13 | −1.07 | −11.02 | 11q12.1 |
| 102 | 218035_s_at | FLJ20273 | −3.9 | 7.3E−15 | 7.73E−13 | −1.05 | −10.79 | 4p13-p12 |
| 15 | 211284_s_at | GRN | −3.87 | 1.64E−18 | 6.97E−16 | −1.44 | −13.89 | 17q21.31 |
| 90 | 223280_x_at | MS4A6A | −3.86 | 1.82E−15 | 2.5E−13 | −1.06 | −11.11 | 11q12.1 |
| 105 | 237255_at | | −3.86 | 1.37E−14 | 1.3E−12 | −1.06 | −10.68 | |
| 220 | 211100_x_at | LILRA2 | −3.86 | 3.91E−12 | 1.6E−10 | −1.1 | −9.41 | 19q13.4 |
| 57 | 200866_s_at | PSAP | −3.84 | 1.89E−15 | 2.58E−13 | −1.27 | −11.7 | 10q21-q22 |
| 80 | 207270_x_at | CMRF35 | −3.84 | 1.56E−14 | 1.44E−12 | −1.32 | −11.23 | 17q25.2 |
| 64 | 202902_s_at | CTSS | −3.81 | 3.18E−15 | 4.04E−13 | −1.26 | −11.56 | 1q21 |
| 106 | 205859_at | LY86 | −3.73 | 1.68E−14 | 1.54E−12 | −1.07 | −10.67 | 6p24.3 |
| 95 | 207857_at | LILRA2 | −3.71 | 8.43E−15 | 8.64E−13 | −1.14 | −11.01 | 19q13.4 |
| 9 | 216041_x_at | GRN | −3.7 | 4.94E−19 | 2.83E−16 | −1.45 | −14.22 | 17q21.32 |
| 82 | 227184_at | | −3.68 | 9.52E−15 | 9.64E−13 | −1.23 | −1.21 | |
| 243 | 233555_s_at | SULF2 | −3.68 | 3.48E−12 | 1.48E−10 | −1 | −9.29 | 20q12-13.2 |
| 145 | 212335_at | GNS | −3.67 | 3.6E−13 | 2.04E−11 | −1.15 | −10.11 | 12q14 |
| 65 | 225059_at | AGTRAP | −3.66 | 2.16E−15 | 2.88E−13 | −1.22 | −11.55 | 1p36.21 |
| 166 | 209288_s_at | CDC42EP3 | −3.66 | 2.67E−13 | 1.63E−11 | −1.02 | −9.93 | 2p21 |
| 88 | 203673_at | GNS | −3.62 | 1.11E−14 | 1.1E−12 | −1.21 | −11.12 | 12q14 |
| 229 | 202284_s_at | CDKN1A | −3.62 | 2.53E−12 | 1.14E−10 | −1.01 | −9.39 | 6p21.2 |
| 290 | 223168_at | ARHU | −3.61 | 7.66E−12 | 2.73E−10 | −0.97 | −9.04 | 1q42.11-q42.3 |
| 162 | 208594_x_at | ILT8 | −3.55 | 6.87E−13 | 3.13E−11 | −1.13 | −9.96 | 19q13.4 |
| 263 | 202877_s_at | C1QR1 | −3.55 | 7.89E−12 | 2.79E−10 | −1.06 | −9.17 | 20p11.21 |
| 33 | 204487_s_at | KCNQ1 | −3.54 | 1.9E−17 | 5.18E−15 | −1.23 | −12.64 | 11p15.5 |
| 282 | 235798_at | | −3.53 | 4.73E−12 | 1.85E−10 | −0.94 | −9.09 | |
| 177 | 205839_at | AOAH | −3.51 | 5.96E−13 | 3.18E−11 | −1.05 | −9.81 | 7p14-p12 |
| 175 | 200838_at | CTSB | −3.5 | 8.17E−13 | 4.16E−11 | −1.11 | −9.84 | 8p22 |
| 174 | 208949_s_at | LGALS3 | −3.49 | 2.52E−13 | 1.55E−11 | −0.98 | −9.84 | 14q21-q22 |
| 255 | 223922_x_at | MS4A6A | −3.48 | 3.41E−12 | 1.46E−10 | −0.96 | −9.21 | 11q12.1 |
| 14 | 200676_x_at | GRN | −3.47 | 1E−18 | 4.62E−16 | −1.41 | −13.92 | 17q21.32 |
| 30 | 224583_at | COTL1 | −3.47 | 4.24E−17 | 1.08E−14 | −1.33 | −12.79 | 16q23.3 |
| 251 | 210184_at | ITGAX | −3.45 | 1.92E−12 | 8.88E−11 | −0.91 | −9.23 | 16p11.2 |
| 112 | 208981_at | PECAM1 | −3.41 | 4.6E−14 | 3.67E−12 | −1.12 | −10.58 | 17q23 |
| 286 | 209286_at | | −3.41 | 3.11E−12 | 1.35E−10 | −0.89 | −9.06 | |
| 17 | 201360_at | CST3 | −3.34 | 1.33E−19 | 9.66E−17 | −1.23 | −13.56 | 20p11.21 |
| 84 | 228532_at | MGC24133 | −3.32 | 6.03E−15 | 6.73E−13 | −1.18 | −11.2 | 1p13.1 |
| 3 | 221841_s_at | | −3.31 | 1.17E−24 | 9.73E−21 | −1.35 | −15.91 | |
| 28 | 203973_s_at | CEBPD | −3.3 | 3.89E−19 | 2.38E−16 | −1.13 | −12.86 | 8p11.2p11.1 |
| 265 | 225685_at | | −3.25 | 2.93E−12 | 1.29E−10 | −0.92 | −9.16 | |
| 246 | 204924_at | TLR2 | −3.24 | 3.51E−12 | 1.48E−10 | −0.99 | −9.26 | 4q32 |
| 92 | 207704_s_at | GAS7 | −3.2 | 1.34E−14 | 127E−12 | −1.2 | −11.07 | 17p |
| 108 | 212334_at | GNS | −3.2 | 3.6E−14 | 2.96E−12 | −1.12 | −10.65 | 12q14 |
| 111 | 217728_at | S100A6 | −3.18 | 3.58E−14 | 2.96E−12 | −1.1 | −10.58 | 1q21 |
| 295 | 229041_s_at | | −3.16 | 4.73E−12 | 1.85E−10 | −0.9 | −9.01 | |
| 73 | 221866_at | TFEB | −3.15 | 7.2E−16 | 1.13E−13 | −1.1 | −11.43 | 8p21 |
| 156 | 204254_s_at | VDR | −3.15 | 1.91E−13 | 1.23E−11 | −1.03 | −10.04 | 12q12-q14 |
| 36 | 202897_at | PTPNS1 | −3.08 | 6.52E−17 | 1.57E−14 | −1.29 | −12.56 | 20p13 |
| 8 | 213503_x_at | ANXA2 | −3.07 | 1.4E−20 | 1.93E−17 | −1.31 | −14.32 | 15q21-q22 |
| 66 | 221059_s_at | CHST6 | −3.07 | 7.26E−16 | 1.13E−13 | −1.13 | −11.54 | 18q22 |
| 77 | 235678_at | | −3.06 | 8.07E−15 | 8.34E−13 | −1.29 | −11.36 | |
| 10 | 201590_x_at | ANXA2 | −3.05 | 3.55E−20 | 3.69E−17 | −1.31 | −14.18 | 15q21-q22 |
| 41 | 200872_at | S100A10 | −3.05 | 7.64E−18 | 2.39E−15 | −1.1 | −12.22 | 1q21 |
| 164 | 210904_s_at | IL13RA1 | −3.03 | 2.59E−13 | 1.59E−11 | −1.02 | −9.95 | Xq24 |
| 20 | 201944_at | HEXB | −3.01 | 3E−18 | 1.16E−15 | −1.35 | −13.47 | 5q13 |
| 146 | 203300_x_at | AP1S2 | −3 | 3.76E−13 | 2.13E−11 | −1.15 | −10.11 | Xp22.31 |
| 197 | 230395_at | | −3 | 1.8E−13 | 1.17E−11 | −0.9 | −9.65 | |
| 37 | 35820_at | GM2A | −2.99 | 5.97E−17 | 1.5E−14 | −1.26 | −12.51 | 5q31.3-q33.1 |
| 16 | 210427_x_at | ANXA2 | −2.95 | 6.87E−20 | 6E−17 | −1.27 | −13.58 | 15q21-q22 |
| 155 | 212807_s_at | SORT1 | −2.92 | 2.27E−13 | 1.42E−11 | −1.05 | −10.04 | 1p21.3-p13.1 |
| 227 | 235670_at | | −2.92 | 8.78E−13 | 4.41E−11 | −0.91 | −9.39 | |
| 273 | 215990_s_at | BCL6 | −2.92 | 5.04E−12 | 1.96E−10 | −0.96 | −9.13 | 3q27 |
| 61 | 204057_at | ICSBP1 | −2.9 | 1.2E−18 | 5.39E−16 | −0.94 | −11.6 | 16q24.1 |
| 200 | 224818_at | | −2.89 | 2.48E−13 | 1.53E−11 | −0.91 | −9.63 | |
| 40 | 204122_at | TYROBP | −2.87 | 9.76E−17 | 2.28E−14 | −1.22 | −12.24 | 19q13.1 |
| 293 | 203769_s_at | STS | −2.87 | 8.88E−12 | 3.07E−10 | −0.98 | −9.02 | Xp22.32 |
| 116 | 225373_at | PP2135 | −2.86 | 3.63E−14 | 2.97E−12 | −1.07 | −10.5 | 10q22.3 |
| 52 | 230264_s_at | AP1S2 | −2.85 | 6.59E−16 | 1.05E−13 | −1.19 | −11.76 | Xp22.31 |
| 81 | 229101_at | | −2.84 | 1.48E−15 | 2.1E−13 | −1.08 | −11.23 | |
| 181 | 202391_at | BASP1 | −2.83 | 3.24E−14 | 2.73E−12 | −0.86 | −9.79 | 5p15.1-p14 |
| 78 | 211067_s_at | GAS7 | −2.81 | 3.39E−15 | 4.24E−13 | −1.18 | −11.34 | 17p |
| 122 | 201105_at | LGALS1 | −2.8 | 6.4E−15 | 6.96E−13 | −0.92 | −10.32 | 22q13.1 |
| 201 | 201531_at | ZFP36 | −2.8 | 7.68E−13 | 3.96E−11 | −0.99 | −9.61 | 19q13.1 |
| 245 | 201642_at | IFNGR2 | −2.79 | 3.86E−12 | 1.59E−10 | −1 | −9.27 | 21q22.11 |
| 169 | 209575_at | IL10RB | −2.77 | 4.92E−13 | 2.67E−11 | −1.06 | −9.88 | 21q22.11 |
| 187 | 208982_at | PECAM1 | −2.77 | 2.23E−13 | 1.4E−11 | −0.94 | −9.74 | 17q23 |
| 43 | 208683_at | CAPN2 | −2.76 | 4.59E−18 | 1.59E−15 | −1.07 | −12.16 | 1q41-q42 |
| 173 | 217897_at | FXYD6 | −2.76 | 2.3E−13 | 1.43E−11 | −0.98 | −9.85 | 11q23.3 |
| 198 | 203388_at | ARRB2 | −2.75 | 8.21E−13 | 4.17E−11 | −1.01 | −9.64 | 19q13 |
| 266 | 225604_s_at | C9of19 | −2.73 | 2.75E−12 | 1.23E−10 | −0.91 | −9.15 | 9p13-p12 |
| 168 | 204774_at | EVI2A | −2.72 | 4.01E−13 | 2.24E−11 | −1.05 | −9.92 | 17q11.2 |
| 237 | 202426_s_at | RXRA | −2.71 | 6.78E−13 | 3.52E−11 | −0.88 | −9.35 | 9q34.3 |
| 153 | 206816_x_at | ANXA2P2 | −2.69 | 2.83E−13 | 1.68E−11 | −1.08 | −10.05 | 9p13 |
| 50 | 204158_s_at | TCIRG1 | −2.68 | 5.3E−16 | 8.97E−14 | −1.18 | −11.76 | 11q13.4-q13.5 |
| 59 | 100839_s_at | CTSB | −2.67 | 3.51E−16 | 6.7E−14 | −1.13 | −11.66 | 8p22 |
| 71 | 232617_at | CTSS | −2.67 | 1.04E−15 | 1.53E−13 | −1.15 | −11.51 | 1q21 |
| 53 | 212737_at | GM2A | −2.65 | 8.37E−16 | 1.26E−13 | −1.21 | −11.76 | 5q31.3-q33.1 |
| 193 | 208906_s_at | CAST | −2.65 | 5.99E−13 | 3.18E−11 | −0.99 | −9.67 | 5q15-q21 |
| 182 | 228234_at | | −2.64 | 2.64E−13 | 1.61E−11 | −0.96 | −9.77 | |
| 58 | 203186_s_at | S100A4 | −2.62 | 1.72E−16 | 3.57E−14 | −1.09 | −11.67 | 1q21 |
| 2 | 202803_s_at | ITGB2 | −2.6 | 9.79E−24 | 4.07E−20 | −1.5 | −16.57 | 21q22.3 |
| 97 | 203140_at | BCL6 | −2.59 | 1.47E−52 | 2.1E−13 | −0.99 | −10.87 | 3q27 |
| 67 | 211612_s_at | IL13RA1 | −2.58 | 5.34E−16 | 8.97E−14 | −1.11 | −11.53 | Xq24 |
| 115 | 205786_s_at | ITGAM | −2.58 | 3.17E−15 | 4.04E−13 | −0.94 | −10.52 | 16p11.2 |
| 280 | 210314_x_at | TNFSF13 | −2.58 | 3.29E−12 | 1.41E−10 | −0.91 | −9.1 | 17p13.1 |
| 22 | 203535_at | S100A9 | −2.56 | 9.12E−23 | 1.89E−19 | −1.03 | −13.19 | 1q21 |
| 135 | 218376_s_at | MICAL | −2.56 | 2.65E−14 | 2.26E−12 | −0.95 | −10.2 | 6q21 |
| 277 | 210785_s_at | C10orf38 | −2.58 | 3.24E−12 | 1.39E−10 | −0.91 | −9.11 | 1p35.2 |
| 179 | 203555_at | PTPN18 | −2.55 | 4.63E−13 | 2.55E−11 | −1.01 | −9.79 | 2q21.1 |
| 45 | 226276_at | LOC153339 | −2.53 | 5.97E−16 | 9.83E−14 | −1.3 | −12.06 | 5q14.1 |
| 185 | 205707_at | IL17R | −2.53 | 2.19E−13 | 1.38E−11 | −0.94 | −9.75 | 22q11.1 |
| 241 | 202896_s_at | PTPNS1 | −2.52 | 1.66E−12 | 7.8E−11 | −0.93 | −9.32 | 20p13 |
| 38 | 214875_x_at | APLP2 | −2.51 | 1.81E−17 | 5.08E−15 | −1.15 | −12.33 | 11q24 |
| 217 | 225602_at | C9orf19 | −2.5 | 8.12E−13 | 4.15E−11 | −0.92 | −9.42 | 9p13-p12 |
| 231 | 204174_at | ALOX5AP | −2.5 | 3.56E−13 | 2.03E−11 | −0.86 | −9.39 | 13q12 |
| 110 | 226063_at | | −2.48 | 8.09E−15 | 8.34E−13 | −1.01 | −10.62 | |
| 68 | 225530_at | moblak | −2.46 | 7.94E−16 | 1.21E−13 | −1.13 | −11.53 | 19p13.3 |
| 262 | 228648_at | LRG | −2.44 | 9.59E−13 | 4.78E−11 | −0.85 | −9.17 | 19p13.3 |
| 261 | 203167_at | TIMP2 | −2.43 | 3.42E−12 | 1.46E−10 | −0.94 | −9.17 | 17q25 |
| 270 | 206050_s_at | RNH | −2.43 | 6.28E−12 | 2.34E−10 | −1 | −9.14 | 11p15.5 |
| 133 | 217024_x_at | | −2.41 | 2.03E−14 | 1.8E−12 | −0.95 | −10.23 | |
| 188 | 218092_s_at | HRB | −2.4 | 8.67E−13 | 4.37E−11 | −1.04 | −9.7 | 2q36 |
| 125 | 227649_s_at | | −2.38 | 9.87E−15 | 9.94E−13 | −0.93 | −10.3 | |
| 136 | 224374_s_at | EMILIN-2 | −2.38 | 9.42E−14 | 6.6E−12 | −1.03 | −10.19 | 18p11.3 |
| 143 | 207467_x_at | CAST | −2.38 | 8.54E−14 | 6.07E−12 | −1 | −10.12 | 5q15-q21 |
| 159 | 203520_s_at | ZFP318 | −2.38 | 8.02E−14 | 5.8E−12 | −0.95 | −9.98 | 6pter-p12.1 |
| 13 | 208702_x_at | APLP2 | −2.37 | 2.68E−20 | 2.99E−17 | −1.26 | −13.98 | 11q24 |
| 23 | 220023_at | APOB48R | −2.37 | 1.45E−19 | 1.01E−16 | −1.16 | −13.14 | 16p11 |
| 298 | 227276_at | TEM7R | −2.37 | 8.13E−12 | 2.85E−10 | −0.95 | −8.98 | 10p12.1 |
| 236 | 203507_at | CD68 | −2.35 | 1.59E−12 | 7.56E−11 | −0.95 | −9.36 | 17p13 |
| 1 | 200871_s_at | PSAP | −2.32 | 3.62E−30 | 6.01E−26 | −1.33 | −17.01 | 10q21-q22 |
| 233 | 238469_at | | −2.32 | 1.23E−12 | 5.94E−11 | −0.93 | −9.37 | |
| 178 | 202670_at | MAP2K1 | −2.31 | 3.22E−13 | 1.87E−11 | −0.99 | −9.81 | 15q22.1-q22.33 |
| 119 | 223502_s_at | TNFSF138 | −2.3 | 3.64E−15 | 4.45E−13 | −0.91 | −10.38 | 13q32-34 |
| 283 | 203299_s_at | AP1S2 | −2.3 | 4.58E−12 | 1.81E−10 | −0.93 | −9.07 | Xp22.31 |
| 147 | 211250_s_at | SH3BP2 | −2.27 | 9.9E−14 | 6.88E−12 | −1 | −10.11 | 4p16.3 |
| 247 | 222774_s_at | NETO2 | −2.26 | 8.06E−13 | 4.13E−11 | −0.87 | −9.26 | |
| 184 | 202096_s_at | BZRP | −2.23 | 2.13E−13 | 1.35E−11 | −0.94 | −9.75 | 22q13.31 |
| 6 | 211404_s_at | APLP2 | −2.22 | 5.83E−23 | 1.61E−19 | −1.31 | −15.14 | 11q24 |
| 128 | 223376_s_at | BRI3 | −2.22 | 7.1E−14 | 5.32E−12 | −1.03 | −10.26 | 7q21.3 |
| 171 | 225695_at | | −2.22 | 1.69E−14 | 1.54E−12 | −0.86 | −9.87 | |
| 149 | 237563_s_at | | −2.2 | 1.06E−13 | 7.29E−12 | −1 | −10.09 | |
| 86 | 206034_at | SERPINB6 | −2.18 | 3.88E−18 | 7.08E−14 | −1.02 | −11.18 | 18q21.3 |
| 248 | 217627_s_at | ACP33 | −2.18 | 3.08E−12 | 1.35E−10 | −0.97 | −8.25 | 15q21-q22 |
| 249 | 226140_a_at | | −2.19 | 6.33E−13 | 3.33E−11 | −0.85 | −9.24 | |
| 51 | 224846_at | LOC92799 | −2.17 | 1.83E−17 | 5.08E−15 | −1.03 | −11.76 | 19q13.13 |
| 203 | 212041_at | ATP6V0D1 | −2.16 | 4.8E−13 | 2.62E−11 | −0.94 | −9.6 | 16q22 |
| 244 | 202192_s_at | GAS7 | −2.16 | 3.99E−13 | 2.24E−11 | −0.84 | −9.27 | 17p |
| 126 | 204971_at | CSTA | −2.15 | 4.08E−16 | 7.36E−14 | −0.84 | −10.29 | 3q21 |
| 276 | 200661_at | PPGB | −2.15 | 1.28E−12 | 6.13E−11 | −0.85 | −9.12 | 20q13.1 |
| 288 | 214574_x_at | LST1 | −2.15 | 3.02E−12 | 1.33E−10 | −0.88 | −9.05 | 6p21.3 |
| 205 | 228685_at | | −2.14 | 1.7E−13 | 1.12E−11 | −0.86 | −9.53 | |
| 75 | 211742_s_at | EVI2B | −2.13 | 3.11E−16 | 6E−14 | −1.06 | −11.41 | 17q11.2 |
| 55 | 200701_at | NPC2 | −2.12 | 4.14E−16 | 7.4E−14 | −1.15 | −11.72 | 14q24.3 |
| 27 | 200742_a_at | CLN2 | −2.11 | 4.76E−19 | 2.83E−16 | −1.14 | −12.88 | 11p15 |
| 117 | 203185_at | RASSF2 | −2.11 | 1.13E−14 | 1.1E−12 | −0.99 | −10.5 | 20pter-p12.1 |
| 93 | 201201_at | CSTB | −2.1 | 7.72E−16 | 1.19E−13 | −1.01 | −11.06 | 21q22.3 |
| 24 | 201954_at | ARPC1B | −2.09 | 4.06E−19 | 2.38E−16 | −1.17 | −13.05 | 7q22.1 |
| 56 | 212501_at | CEBPB | −2.09 | 2.93E−18 | 1.16E−11 | −0.98 | −11.71 | 20q13.1 |
| 232 | 221875_x_at | HLA-F | −2.09 | 6.07E−13 | 3.2E−15 | −0.89 | −9.38 | 6p21.3 |
| 26 | 210872_x_at | GAS7 | −2.08 | 8.67E−19 | 4.24E−16 | −1.17 | −12.91 | 17p |
| 226 | 202625_at | LYN | −2.08 | 4.29E−13 | 2.39E−11 | −0.87 | −9.4 | 8q13 |
| 240 | 225844_at | POLE4 | −2.08 | 2.8E−12 | 1.24E−10 | −0.99 | −9.32 | 2p12 |
| 165 | 209901_x_at | AIF1 | −2.07 | 5.17E−14 | 4.04E−12 | −0.92 | −9.95 | 6p21.3 |
| 160 | 210980_s_at | ASAH1 | −2.06 | 8.48E−14 | 6.05E−12 | −0.95 | −9.98 | 8p22-p21.3 |
| 234 | 223145_a_at | FLJ10342 | −2.06 | 1.11E−12 | 5.4E−11 | −0.92 | −9.36 | 6q15 |
| 7 | 200886_s_at | PGAM1 | −2.05 | 2.93E−21 | 4.42E−18 | −1.20 | −14.4 | 10q25.3 |
| 292 | 202957_at | HCLS1 | −2.03 | 5.24E−12 | 2.02E−10 | −0.92 | −9.03 | 3q13 |
| 141 | 200808_s_at | ZYX | −2.02 | 2.43E−14 | 2.11E−12 | −0.93 | −10.13 | 7q32 |
| 213 | 202484_s_at | MBD2 | −2.02 | 1.03E−12 | 5.09E−11 | −0.96 | −9.48 | 18q21 |
| 157 | 226353_at | SPPL2A | −2 | >6.1E−14 | 4.67E−12 | −0.95 | −10.03 | 15q15.3 |
| 172 | 226219_at | LOC257106 | −2 | 1.46E−13 | 9.78E−12 | −0.95 | −9.85 | 1q23.1 |
| 5 | 208885_at | LCP1 | −1.97 | 3.67E−23 | 1.22E−19 | −1.31 | −15.19 | 13q14.3 |
| 252 | 202228_s_at | SDFR1 | −1.96 | 9.91E−13 | 4.92E−11 | −0.87 | −9.23 | 15q22 |
| 297 | 204479_at | OSTF1 | −1.96 | 7.11E−12 | 2.58E−10 | −0.93 | −8.99 | 12q24.1-24.2 |
| 260 | 206994_at | CST4 | −1.95 | 6.06E−12 | 4.66E−12 | −0.75 | −9.18 | 20p11.21 |
| 91 | 208704_x_at | APLP2 | −1.93 | 1.52E−16 | 3.21E−14 | −0.96 | −11.09 | 11q24 |
| 223 | 223058_at | MGC11034 | −1.93 | 1.04E−14 | 1.04E−12 | −0.75 | −9.41 | 10p13 |
| 60 | 208248_x_at | APLP2 | −1.9 | 1.76E−17 | 5.05E−15 | −1.01 | −11.65 | 11q24 |
| 99 | 203416_at | CD53 | −1.9 | 4.2E−15 | 5E−13 | −1.03 | −10.82 | 1p13 |
| 107 | 204265_a_at | C6orf9 | −1.9 | 4.21E−15 | 5E−13 | −0.98 | −10.65 | 6q21.3 |
| 114 | 221492_s_at | APG3 | −1.89 | 1.83E−14 | 1.64E−12 | −1.03 | −10.54 | 3p13.13 |
| 214 | 34689_at | TREX1 | −1.88 | 4.51E−13 | 2.5E−11 | −0.9 | −9.47 | 3p21.3-p21.2 |
| 279 | 202388_at | RGS2 | −1.85 | 9.56E−14 | 6.67E−12 | −0.75 | −9.11 | 1q31 |
| 176 | 209166_s_at | MAN2B1 | −1.84 | 3.38E−14 | 2.81E−12 | −0.87 | −9.83 | 19cen-q13.1 |
| 257 | 229860_x_at | | −1.84 | 2.75E−13 | 1.66E−11 | −0.8 | −9.19 | |
| 183 | 210740_s_at | ITPK1 | −1.8 | 2.76E−13 | 1.66E−11 | −0.96 | −9.77 | 14q31 |
| 216 | 201136_at | PLP2 | −1.8 | 1.15E−13 | 7.88E−12 | −0.82 | −9.43 | Xp11.23 |
| 85 | 200975_at | PPT1 | −1.79 | 5.87E−16 | 9.75E−14 | −1.03 | −11.19 | 1p32 |
| 158 | 225750_at | | −1.78 | 1.26E−14 | 1.21E−12 | −0.88 | −10.03 | |
| 49 | 201720_s_at | LAPTM5 | −1.77 | 5.07E−19 | 2.63E−16 | −0.98 | −11.94 | 1p34 |
| 253 | 200078_s_at-HG-U133A | ATP6V0B | −1.77 | 1.78E−12 | 8.29E−11 | −0.91 | −9.23 | 1p32.3 |
| 289 | 209073_s_at | NUMB | −1.77 | 1.98E−12 | 9.12E−11 | −0.85 | −9.04 | 14q24.3 |
| 47 | 201444_s_at | ATP6IP2 | −1.76 | 7.19E−18 | 2.3E−15 | −1.05 | −11.99 | Xq21 |
| 204 | 211926_s_at | MYH9 | −1.75 | 1.42E−13 | 9.52E−12 | −0.87 | −9.58 | 22q13.1 |
| 134 | 208913_at | GGA2 | −1.74 | 9.3E−15 | 9.48E−13 | −0.91 | −10.21 | 16p12 |
| 170 | 203175_at | ARHG | −1.74 | 1.7E−14 | 1.54E−12 | −0.86 | −9.88 | 11p15.5-p15.4 |
| 103 | 223626_x_at | FAM14A | −1.73 | 1.33E−16 | 2.99E−14 | −0.9 | −10.77 | 14q32.13 |
| 113 | 213702_x_at | AAH1 | −1.73 | 4.85E−15 | 5.6E−13 | −0.97 | −10.55 | 8p22-p21.3 |
| 230 | 209393_s_at | EIF4EL3 | −1.72 | 2.86E−13 | 1.68E−11 | −0.85 | −9.39 | 2q37.1 |
| 32 | 200743_s_at | CLN2 | −1.71 | 4.6E−20 | 4.5E−17 | −1.06 | −12.65 | 11p15 |
| 104 | 208959_s_at | TXNDC4 | −1.69 | 2.13E−15 | 2.86E−13 | −0.96 | −10.69 | 9q22.33 |
| 120 | 200625_s_at | CAP | −1.69 | 4.93E−15 | 5.65E−13 | −0.92 | −10.33 | 1p34.1 |
| 272 | 202605_at | GUSB | −1.69 | 2.84E−13 | 1.68E−11 | −0.79 | −9.13 | 7q21.11 |
| 39 | 208736_at | ARPC3 | −1.67 | 6.63E−20 | 6E−17 | −1.01 | −12.33 | 12q24.11 |
| 148 | 212350_at | TBC1D1 | −1.66 | 1.17E−14 | 1.13E−12 | −0.89 | −10.1 | 4p14 |
| 144 | 214665_s_at | | −1.64 | 1.22E−14 | 1.17E−12 | −0.9 | −10.11 | |
| 98 | 213798_s_at | CAP | −1.63 | 3.58E−16 | 6.76E−14 | −0.94 | −10.84 | 1p34.1 |
| 281 | 215952_s_at | | −1.63 | 1.39E−13 | 9.38E−12 | −0.76 | −9.1 | |
| 287 | 200654_at | P4HB | −1.61 | 1.47E−13 | 9.78E−12 | −0.75 | −9.06 | 17q25 |
| 87 | 207988_s_at | ARPC2 | −1.6 | 1.14E−16 | 2.63E−14 | −0.96 | −11.13 | 2q36.1 |
| 274 | 200055_at-HG-U133B | TAF10 | −1.57 | 8.61E−13 | 4.35E−11 | −0.83 | −9.13 | 11p15.3 |
| 219 | 224573_at | MGC49942 | −1.54 | 7.53E−14 | 5.55E−12 | −0.81 | −9.42 | 17p13.2 |
| 189 | 222992_s_at | NDUFB9 | −1.49 | 6.08E−15 | 6.73E−13 | −0.79 | −9.68 | 8q13.3 |
| 238 | 200645_at | GABARAP | −1.49 | 8.81E−14 | 6.23E−12 | −0.8 | −9.35 | 17p13.2 |
| 207 | 200640_at | YWHAZ | −1.48 | 4.34E−14 | 3.49E−12 | −0.81 | −9.5 | 8q23.1 |
| 215 | 47105_at | FLJ20399 | −1.48 | 7.27E−14 | 5.42E−12 | −0.81 | −9.44 | 16q22.1 |
| 191 | 201858_s_at | PRG1 | −1.46 | 4.4E−16 | 7.78E−14 | −0.74 | −9.68 | 10q22.1 |
| 221 | 201290_at | SPC18 | −1.44 | 3.83E−14 | 3.1E−12 | −0.79 | −9.41 | 15q24.3 |
| 291 | 209563_x_at | CALM1 | −1.44 | 8.11E−14 | 5.83E−12 | −0.73 | −9.04 | 14q24-q31 |
| 29 | 207157_s_at | GNG5 | −1.42 | 3.92E−22 | 6.51E−19 | −1.01 | −12.85 | 1p22 |
| 123 | 213603_s_at | RAC2 | −1.39 | 6.11E−18 | 1.99E−15 | −0.78 | −10.31 | 22q13.1 |
| 118 | 213513_x_at | ARPC2 | −1.38 | 4.84E−16 | 8.4E−14 | −0.88 | −10.4 | 2q36.1 |
| 192 | 223880_x_at | C20orf24 | −1.38 | 9.93E−15 | 9.94E−13 | −0.8 | −9.67 | 20q11.22 |
| 222 | 224625_x_at | PNN | −1.38 | 6.37E−15 | 6.07E−13 | −0.74 | −9.41 | 14q13.2 |
| 109 | 200738_s_at | PGK1 | −1.37 | 1.49E−16 | 3.17E−14 | −0.88 | −10.64 | Xq13 |
| 199 | 217835_x_at | C20orf24 | −1.36 | 1.13E−14 | 1.1E−12 | −0.8 | −9.64 | 20q11.22 |
| 254 | 208612_at | GRP58 | −1.36 | 1.46E−14 | 1.37E−12 | −0.73 | −9.23 | 15q15 |
| 194 | 217733_s_at | TMSB10 | −1.32 | 3.49E−15 | 4.33E−13 | −0.78 | −9.67 | 2p11.2 |
| 268 | 200088_s_at-HG-U133B | COX4I1 | −1.26 | 2.58E−14 | 2.21E−12 | −0.73 | −9.14 | 16q22-qter |

| | | | Sequence | | | | | |
|---|---|---|---|---|---|---|---|---|
| | Sequence | Transcript | Derived | Sequence | Sequence | Unigene | Cluster | Locus |
| Number | Type | ID | From | ID | Source | Accession | Type | Link |
| 239 | Exemplar | Hs.105928.4 | AF009644.1 | g2662447 | GenBank | Hs.105928 | fulllength | 11025 |
| | sequence | | | | | | | |
| 278 | Exemplar | Hs.105928.0 | AF009635.1 | g2662429 | GenBank | Hs.105928 | fulllength | 11025 |
| | sequence | | | | | | | |
| 132 | Exemplar | Hs.105928.2 | AF009634.1 | g2662427 | GenBank | Hs.105928 | fulllength | 11025 |
| | sequence | | | | | | | |
| 275 | Consensus | Hs.306542.0 | R94644 | Hs.306542.0 | GenBank | Hs.81800 | fulllength | 1462 |
| | sequence | | | | | | | |
| 96 | Exemplar | Hs.159237.0 | NM_002115.1 | g4504394 | RefSeq | Hs.159237 | fulllength | 3101 |
| | sequence | | | | | | | |
| 89 | Consensus | Hs.288581.0 | T64884 | Hs.288581.0.S1 | GenBank | Hs.288581 | | 219972 |
| | sequence | | | | | | | |
| 264 | Exemplar | Hs.1799.0 | NM_001766.1 | g4502648 | RefSeq | Hs.1799 | fulllength | 912 |
| | sequence | | | | | | | |
| 210 | Exemplar | Hs.78864.1 | U90939.1 | g2149627 | GenBank | Hs.78864 | fulllength | 2212 |
| | sequence | | | | | | | |
| 83 | Exemplar | Hs.7934.0 | NM_004235.1 | g4758321 | RefSeq | Hs.356370 | fulllength | 9314 |
| | sequence | | | | | | | |
| 299 | Exemplar | Hs.105928.1 | AF009643.1 | g2662445 | GenBank | Hs.105928 | fulllength | 11025 |
| | sequence | | | | | | | |
| 19 | Consensus | Hs.288581.0 | T64884 | Hs.288581.0.S1 | GenBank | Hs.288581 | | 219972 |
| | sequence | | | | | | | |
| 35 | Exemplar | Hs.288181.0 | NM_004390.1 | g4758095 | RefSeq | Hs.288181 | fulllength | 1512 |
| | sequence | | | | | | | |
| 211 | Exemplar | Hs.194784.0 | NM_006065.1 | g5174678 | RefSeq | Hs.194784 | fulllength | 10326 |
| | sequence | | | | | | | |
| 259 | Exemplar | Hs.122591.0 | NM_013439.1 | g7305384 | RefSeq | Hs.122591 | fulllength | 29992 |
| | sequence | | | | | | | |
| 21 | Exemplar | Hs.14623.0 | NM_006332.1 | g5453695 | RefSeq | Hs.14623 | fulllength | 10437 |
| | sequence | | | | | | | |
| 54 | Consensus | Hs.122591.1 | AJ400843.1 | Hs.122591.1 | GenBank | Hs.122591 | fulllength | 29992 |
| | sequence | | | | | | | |
| 76 | Consensus | Hs.16987.0 | AW135013 | Hs.169487.0 | GenBank | Hs.169487 | fulllength | 9935 |
| | sequence | | | | | | | |
| 69 | Exemplar | Hs.297681.2 | AF119873.1 | g770182 | GenBank | Hs.297681 | fulllength | 5265 |
| | sequence | | | | | | | |
| 267 | Consensus | Hs.109438.0 | AF052169.1 | Hs.109438.0 | GenBank | Hs.109438 | fulllength | 115207 |
| | sequence | | | | | | | |
| 100 | Exemplar | Hs.574.0 | D26054.1 | g439225 | GenBank | Hs.574 | fulllength | 2203 |
| | sequence | | | | | | | |
| 202 | Exemplar | Hs.27954.1 | L25259.1 | g416368 | GenBank | Hs.27954 | fulllength | 942 |
| | sequence | | | | | | | |
| 44 | Exemplar | Hs.46.0 | D10202.1 | g219975 | GenBank | Hs.46 | fulllength | 5724 |
| | sequence | | | | | | | |
| 224 | Consensus | Hs.23262.0 | NM_005615.1 | Hs.23262.0 | GenBank | Hs.23262 | fulllength | 6039 |
| | sequence | | | | | | | |
| 31 | Consensus | Hs.81800.4 | J02814.1 | Hs.81800.4_RC | GenBank | Hs.81800 | fulllength | 1462 |
| | sequence | | | | | | | |
| 152 | Exemplar | Hs.97199.0 | NM_012072.2 | g11496985 | RefSeq | Hs.97199 | fulllength | 22918 |
| | sequence | | | | | | | |
| 250 | Exemplar | Hs.1583.0 | NM_000265.1 | g4557784 | RefSeq | Hs.1583 | fulllength | 4687 |
| | sequence | | | | | | | |
| 25 | Exemplar | Hs.81800.0 | NM_004385.1 | g4758081 | RefSeq | Hs.81800 | fulllength | 1462 |
| | sequence | | | | | | | |
| 48 | Exemplar | Hs.169487.0 | NM_005461.1 | g4885446 | RefSeq | Hs.169487 | fulllength | 9935 |
| | sequence | | | | | | | |
| 18 | Exemplar | Hs.106747.0 | NM_021626.1 | g11055991 | RefSeq | Hs.106747 | fulllength | 59342 |
| | sequence | | | | | | | |
| 101 | Exemplar | Hs.53155.0 | NM_002621.1 | g4505736 | RefSeq | Hs.53155 | fulllength | 5199 |
| | sequence | | | | | | | |
| 256 | Exemplar | Hs.2161.0 | NM_001736.1 | g4502508 | RefSeq | Hs.2161 | fulllength | 728 |
| | sequence | | | | | | | |
| 11 | Exemplar | Hs.300711.0 | NM_001154.2 | g4809273 | RefSeq | Hs.300711 | fulllength | 308 |
| | sequence | | | | | | | |
| 70 | Consensus | Hs.79876.0 | AU138166 | Hs.79876.0.A2 | GenBank | Hs.79876 | fulllength | 412 |
| | sequence | | | | | | | |
| 34 | Consensus | Hs.95549.0 | AW241742 | Hs.95549.0.A1 | GenBank | Hs.95549 | fulllength | 54502 |
| | sequence | | | | | | | |
| 228 | Exemplar | Hs.252136.0 | NM_02003.2 | g8051583 | RefSeq | Hs.252136 | fulllength | 2219 |
| | sequence | | | | | | | |
| 284 | Consensus | Hs.27954.0 | BG236280 | Hs.27954.0.S2 | GenBank | Hs.27954 | fulllength | 942 |
| | sequence | | | | | | | |
| 137 | Exemplar | Hs.135201.0 | NM_022162.1 | g11545911 | RefSeq | Hs.135201 | fulllength | 64127 |
| | sequence | | | | | | | |
| 42 | Exemplar | Hs.949.0 | BC001606.1 | g12804408 | GenBank | Hs.949 | fulllength | 4688 |
| | sequence | | | | | | | |
| 12 | Exemplar | Hs.743.0 | NM_004106.1 | g4758343 | RefSeq | Hs.433300 | fulllength | 2207 |
| | sequence | | | | | | | |
| 4 | Exemplar | Hs.1422.0 | NM_005248.1 | g4885234 | RefSeq | Hs.1422 | fulllength | 2268 |
| | sequence | | | | | | | |
| 294 | Consensus | Hs.293616.0 | AW043782 | Hs.293616.0 | GenBank | Hs.205865 | | 143458 |
| | sequence | | | | | | | |
| 167 | Exemplar | Hs.78146.0 | M37780.1 | g187239 | GenBank | Hs.78146 | fulllength | 5175 |
| | sequence | | | | | | | |
| 46 | Exemplar | Hs.170310.0 | NM_017424.1 | g8393092 | RefSeq | Hs.170310 | fulllength | 51816 |
| | sequence | | | | | | | |
| 130 | Exemplar | g189537 | M80436.1 | g189537 | GenBank | Hs.46 | fulllength | 5724 |
| | sequence | | | | | | | |
| 72 | Exemplar | Hs.94498.2 | U82276.1 | g1907318 | GenBank | Hs.94498 | fulllength | 11027 |
| | sequence | | | | | | | |
| 79 | Exemplar | Hs.23889.0 | NM_015474.1 | g7661593 | RefSeq | Hs.23889 | fulllength | 25939 |
| | sequence | | | | | | | |
| 121 | Exemplar | Hs.26944.0 | NM_006176.1 | g5453799 | RefSeq | Hs.26944 | fulllength | 4900 |
| | sequence | | | | | | | |
| 94 | Exemplar | g13649404 | AF237908.1 | g13649404 | GenBank | Hs.17914 | fulllength | 64231 |
| | sequence | | | | | | | |
| 102 | Exemplar | Hs.95549.0 | NM_019027.1 | g9506670 | RefSeq | Hs.95549 | fulllength | 54502 |
| | sequence | | | | | | | |
| 15 | Exemplar | Hs.180577.1 | BC000324.1 | g12653114 | GenBank | Hs.180577 | fulllength | 2896 |
| | sequence | | | | | | | |
| 90 | Exemplar | Hs.17914.1 | AF253977.1 | g12005800 | GenBank | Hs.17914 | fulllength | 64231 |
| | sequence | | | | | | | |
| 105 | Consensus | Hs.58879.0 | BF222867 | Hs.58879.0_RC | GenBank | Hs.58879 | est | |
| | sequence | | | | | | | |
| 220 | Exemplar | Hs.94498.1 | U82278.1 | g1907322 | GenBank | Hs.94498 | fulllength | 11027 |
| | sequence | | | | | | | |
| 57 | Exemplar | Hs.78575.0 | M3222.1 | g337761 | GenBank | Hs.406455 | fulllength | 5660 |
| | sequence | | | | | | | |
| 80 | Exemplar | Hs.2605.0 | NM_006678.1 | g5729775 | RefSeq | Hs.2605 | fulllength | 10871 |
| | sequence | | | | | | | |
| 64 | Exemplar | Hs.181301.0 | NM_004079.1 | g4758097 | RefSeq | Hs.181301 | fulllength | 1520 |
| | sequence | | | | | | | |
| 106 | Exemplar | Hs.184018.0 | NM_004271.1 | g4758707 | RefSeq | Hs.184018 | fulllength | 9450 |
| | sequence | | | | | | | |
| 95 | Exemplar | Hs.94498.0 | NM_006866.1 | g5803067 | RefSeq | Hs.94498 | fulllength | 11027 |
| | sequence | | | | | | | |
| 9 | Consensus | Hs.180577.2 | AK023348.1 | Hs.180577.2.S1 | GenBank | Hs.180577 | fulllength | 2896 |
| | sequence | | | | | | | |
| 82 | Consensus | Hs.77542.0 | BF508702 | Hs.77542.0_RC | GenBank | Hs.77542 | est | |
| | sequence | | | | | | | |
| 243 | Consensus | Hs.43857.1 | ALD34418 | Hs.43857.1.S1 | GenBank | Hs.43857 | fulllength | 55959 |
| | sequence | | | | | | | |
| 145 | Consensus | Hs.164036.0 | AW167793 | Hs.164036.0.S1 | GenBank | Hs.334534 | fulllength | 2799 |
| | sequence | | | | | | | |
| 65 | Consensus | Hs.12854.0 | BE875567 | Hs.12854.0 | GenBank | Hs.12854 | fulllength | 57085 |
| | sequence | | | | | | | |
| 166 | Exemplar | Hs.260024.0 | AL136842.1 | g6807668 | GenBank | Hs.260024 | fulllength | 10602 |
| | sequence | | | | | | | |
| 88 | Exemplar | Hs.321070.2 | NM_002076.1 | g4504060 | RefSeq | Hs.334534 | fulllength | 2799 |
| | sequence | | | | | | | |
| 229 | Exemplar | Hs.179665.0 | NM_000389.1 | g11386202 | RefSeq | Hs.179665 | fulllength | 1026 |
| | sequence | | | | | | | |
| 290 | Consensus | Hs.20252.0 | AL096776 | Hs.20252.0_RC | GenBank | Hs.20252 | fulllength | 58480 |
| | sequence | | | | | | | |
| 162 | Exemplar | Hs.306230.0 | NM_024318.1 | g13324689 | RefSeq | Hs.306230 | fulllength | 79168 |
| | sequence | | | | | | | |
| 263 | Consensus | Hs.97199.0 | W72082 | Hs.97199.0.S2 | GenBank | Hs.97199 | fulllength | 22918 |
| | sequence | | | | | | | |
| 33 | Exemplar | Hs.156115.0 | NM_000218.1 | g4557688 | RefSeq | Hs.158115 | fulllength | 3784 |
| | sequence | | | | | | | |
| 282 | Consensus | Hs.34650.0 | BE671123 | Hs.34650.0.A1 | GenBank | Hs.34650 | est | |
| | sequence | | | | | | | |
| 177 | Exemplar | Hs.82542.0 | NM_001637.1 | g4502114 | RefSeq | Hs.82542 | fulllength | 313 |
| | sequence | | | | | | | |
| 175 | Exemplar | Hs.297939.0 | NM_001908.1 | g4503138 | RefSeq | Hs.297939 | fulllength | 1508 |
| | sequence | | | | | | | |
| 174 | Exemplar | Hs.621.0 | BC001120.1 | g12654570 | GenBank | Hs.621 | fulllength | 3958 |
| | sequence | | | | | | | |
| 255 | Exemplar | Hs.17914.2 | AB013104.1 | g11559215 | GenBank | Hs.17914 | fulllength | 64231 |
| | sequence | | | | | | | |
| 14 | Exemplar | Hs.180577.0 | NM_002087.1 | g4504150 | RefSeq | Hs.180577 | fulllength | 2898 |
| | sequence | | | | | | | |
| 30 | Consensus | Hs.289092.0 | AL565621 | Hs.289092.0 | GenBank | Hs.289092 | fulllength | 23406 |
| | sequence | | | | | | | |
| 251 | Exemplar | Hs.51077.0 | M81695.1 | g487829 | GenBank | Hs.51077 | fulllength | 3687 |
| | sequence | | | | | | | |
| 112 | Consensus | Hs.78146.0 | AW574504 | Hs.78146.0.S3 | GenBank | Hs.78146 | fulllength | 5175 |
| | sequence | | | | | | | |
| 286 | Consensus | Hs.260024.0 | AI754416 | Hs.260024.0.A3 | GenBank | Hs.352554 | | |
| | sequence | | | | | | | |
| 17 | Exemplar | Hs.135084.0 | NM_000099.1 | g4503106 | RefSeq | Hs.304682 | fulllength | 1471 |
| | sequence | | | | | | | |
| 84 | Consensus | Hs.288010.0 | AW662189 | Hs.288010.0_RC | GenBank | Hs.288010 | fulllength | 128346 |
| | sequence | | | | | | | |
| 3 | Consensus | Hs.7934.1 | BF514079 | Hs.7934.1_RC | GenBank | Hs.376206 | | |
| | sequence | | | | | | | |
| 28 | Exemplar | Hs.76722.0 | NM_005195.1 | g4885130 | RefSeq | Hs.76722 | fulllength | 1052 |
| | sequence | | | | | | | |
| 265 | Consensus | Hs.6774.0 | AI801777 | Hs.6774.0.A1 | GenBank | Hs.352554 | | |
| | sequence | | | | | | | |
| 246 | Exemplar | Hs.63668.0 | NM_003264.1 | g4507528 | RefSeq | Hs.63658 | fulllength | 7097 |
| | sequence | | | | | | | |
| 92 | Exemplar | Hs.226133.1 | NM_003644.1 | g4503920 | RefSeq | Hs.226133 | fulllength | 8522 |
| | sequence | | | | | | | |
| 108 | Consensus | Hs.164036.0 | AW167793 | Hs.164036.0.S1 | GenBank | Hs.334534 | fulllength | 2799 |
| | sequence | | | | | | | |
| 111 | Exemplar | Hs.275243.0 | NM_014624.2 | g9845517 | RefSeq | Hs.275243 | fulllength | 6277 |
| | sequence | | | | | | | |
| 295 | Consensus | Hs.72080.0 | AW303397 | Hs.72080.0_RC | GenBank | Hs.72080 | | |
| | sequence | | | | | | | |
| 73 | Consensus | Hs.23391.0 | AL035588 | Hs.23391.0 | GenBank | Hs.23391 | fulllength | 7942 |
| | sequence | | | | | | | |
| 156 | Exemplar | Hs.2062.0 | NM_000376.1 | g4507882 | RefSeq | Hs.2062 | fulllength | 7421 |
| | sequence | | | | | | | |
| 36 | Exemplar | Hs.156114.0 | AB023430.1 | g6518912 | GenBank | Hs.156114 | fulllength | 140885 |
| | sequence | | | | | | | |
| 8 | Consensus | Hs.217493.4 | BE908217 | Hs.217493.4.S1 | GenBank | Hs.217493 | fulllength | 302 |
| | sequence | | | | | | | |
| 66 | Exemplar | Hs.157439.0 | NM_021615.1 | g11055975 | RefSeq | Hs.157439 | fulllength | 4166 |
| | sequence | | | | | | | |
| 77 | Consensus | Hs.39972.0 | AI864053 | Hs.39972.0_RC | GenBank | Hs.39972 | est | |
| | sequence | | | | | | | |
| 10 | Exemplar | Hs.217493.0 | NM_004039.1 | g4757755 | RefSeq | Hs.217493 | fulllength | 302 |
| | sequence | | | | | | | |
| 41 | Exemplar | Hs.119301.0 | NM_002966.1 | g4506760 | RefSeq | Hs.400250 | fulllength | 6281 |
| | sequence | | | | | | | |
| 164 | Exemplar | Hs.285115.1 | U81380.2 | g5174767 | GenBank | Hs.285115 | fulllength | 3597 |
| | sequence | | | | | | | |
| 20 | Exemplar | Hs.51043.0 | NM_000521.2 | g13128866 | RefSeq | Hs.51043 | fulllength | 3074 |
| | sequence | | | | | | | |
| 146 | Exemplar | Hs.40368.0 | NM_003916.1 | g4506956 | RefSeq | Hs.40368 | fulllength | 8905 |
| | sequence | | | | | | | |
| 197 | Consensus | Hs.14411.0 | AI536637 | Hs.14411.0.A1 | GenBank | Hs.14411 | | |
| | sequence | | | | | | | |
| 37 | Consensus | 4898374 | X62078 | 4898374 | GenBank | Hs.289082 | fulllength | 2760 |
| | sequence | | | | | | | |
| 16 | Exemplar | Hs.217493.2 | BC001388.1 | g12655074 | GenBank | Hs.217493 | fulllength | 302 |
| | sequence | | | | | | | |
| 155 | Consensus | Hs.281706.1 | BE742268 | Hs.281706.1_RC | GenBank | Hs.351872 | fulllength | 6272 |
| | sequence | | | | | | | |
| 227 | Consensus | Hs.28788.0 | AI916948 | Hs.28788.0 | GenBank | Hs.28788 | est | |
| | sequence | | | | | | | |
| 273 | Consensus | Hs.155024.1 | S67779.1 | Hs.155024.1.S1 | GenBank | Hs.155024 | fulllength | 604 |
| | sequence | | | | | | | |
| 61 | Consensus | Hs.14453.0 | AI073984 | Hs.14453.0.S2 | GenBank | Hs.14453 | fulllength | 3394 |
| | sequence | | | | | | | |
| 200 | Consensus | Hs.180372.0 | BE622952 | Hs.180372.0 | GenBank | Hs.394609 | | |
| | sequence | | | | | | | |
| 40 | Exemplar | Hs.9963.0 | NM_003332.1 | g4507754 | RefSeq | Hs.9963 | fulllength | 7305 |
| | sequence | | | | | | | |
| 293 | Exemplar | Hs.79876.0 | NM_000351.2 | g13162281 | RefSeq | Hs.79876 | fulllength | 412 |
| | sequence | | | | | | | |
| 116 | Consensus | Hs.132569.0 | BE271644 | Hs.132569.0.A1 | GenBank | Hs.132569 | fulllength | 64115 |
| | sequence | | | | | | | |
| 52 | Consensus | Hs.40368.3 | AA551090 | Hs.40368.3 | GenBank | Hs.40368 | fulllength | 8905 |
| | sequence | | | | | | | |
| 81 | Consensus | Hs.177376.0 | AI963142 | Hs.177376.0.A1 | GenBank | Hs.177376 | est | |
| | sequence | | | | | | | |
| 181 | Exemplar | Hs.79516.0 | NM_006317.1 | g5453749 | RefSeq | Hs.79516 | fulllength | 10409 |
| | sequence | | | | | | | |
| 78 | Exemplar | g13623656 | BC006454.1 | g13623656 | GenBank | Hs.226133 | fulllength | 8522 |
| | sequence | | | | | | | |
| 122 | Exemplar | Hs.227751.0 | NM_002305.2 | g6006015 | RefSeq | Hs.382367 | fulllength | 3956 |
| | sequence | | | | | | | |
| 201 | Exemplar | Hs.1665.0 | NM_003407.1 | g4507960 | RefSeq | Hs.243586 | fulllength | 7538 |
| | sequence | | | | | | | |
| 245 | Exemplar | Hs.177559.0 | NM_005534.1 | g5031782 | RefSeq | Hs.177559 | fulllength | 3460 |
| | sequence | | | | | | | |
| 169 | Exemplar | Hs.173936.0 | BC001903.1 | g12804902 | GenBank | Hs.173936 | fulllength | 3588 |
| | sequence | | | | | | | |
| 187 | Consensus | Hs.78146.0 | AW574504 | Hs.78146.0.S3 | GenBank | Hs.78146 | fulllength | 5175 |
| | sequence | | | | | | | |
| 43 | Exemplar | Hs.76288.0 | M23254.1 | g511636 | GenBank | Hs.76288 | fulllength | 824 |
| | sequence | | | | | | | |
| 173 | Exemplar | Hs.3807.0 | NM_022003.1 | g11612654 | RefSeq | Hs.3807 | fulllength | 53826 |
| | sequence | | | | | | | |
| 198 | Exemplar | Hs.18142.0 | NM_004313.1 | g4757779 | RefSeq | Hs.18142 | fulllength | 409 |
| | sequence | | | | | | | |
| 266 | Consensus | Hs.75544.1 | AA284532 | Hs.75544.1.S1 | GenBank | Hs.302766 | fulllength | 152007 |
| | sequence | | | | | | | |
| 168 | Exemplar | Hs.70499.0 | NM_014210.1 | g7657074 | RefSeq | Hs.70499 | fulllength | 2123 |
| | sequence | | | | | | | |
| 237 | Consensus | Hs.20064.0 | NM_002957.2 | Hs.20084.0 | GenBank | Hs.20084 | fulllength | 6256 |
| | sequence | | | | | | | |
| 153 | Exemplar | Hs.217493.1 | M62898.1 | g187147 | GenBank | Hs.406239 | fulllength | 304 |
| | sequence | | | | | | | |
| 50 | Exemplar | Hs.46465.1 | NM_006019.1 | g5174620 | RefSeq | Hs.46465 | fulllength | 10312 |
| | sequence | | | | | | | |
| 59 | Exemplar | Hs.297939.0 | NM_001906.1 | g4503138 | RefSeq | Hs.297939 | fulllength | 1508 |
| | sequence | | | | | | | |
| 71 | Consensus | Hs.181301.1 | AK024855.1 | Hs.181301.1 | GenBank | Hs.181301 | fulllength | 1520 |
| | sequence | | | | | | | |
| 53 | Consensus | Hs.278242.3 | AL513583 | Hs.278242.3 | GenBank | Hs.289082 | fulllength | 2760 |
| | sequence | | | | | | | |
| 193 | Exemplar | Hs.279607.1 | AF327443.1 | g12056961 | GenBank | Hs.359682 | fulllength | 831 |
| | sequence | | | | | | | |
| 182 | Consensus | Hs.114055.0 | AI423165 | Hs.114055.0.A1 | GenBank | Hs.390639 | | |
| | sequence | | | | | | | |
| 58 | Exemplar | Hs.81256.0 | NM_002961.2 | g9844514 | RefSeq | Hs.81256 | fulllength | 6275 |
| | sequence | | | | | | | |
| 2 | Exemplar | Hs.83968.0 | NM_000211.1 | g4557885 | RefSeq | Hs.83968 | fulllength | 3689 |
| | sequence | | | | | | | |
| 97 | Exemplar | Hs.155024.0 | NM_00106.1 | g4502383 | RefSeq | Hs.155024 | fulllength | 604 |
| | sequence | | | | | | | |
| 67 | Exemplar | g1695875 | U62858.1 | g1695875 | GenBank | Hs.285115 | fulllength | 3597 |
| | sequence | | | | | | | |
| 115 | Exemplar | Hs.172631.0 | NM_000632.2 | g6006013 | RefSeq | Hs.172631 | fulllength | 3684 |
| | sequence | | | | | | | |
| 280 | Exemplar | Hs.54673.1 | AF114013.1 | g7328557 | GenBank | Hs.54673 | fulllength | 8741 |
| | sequence | | | | | | | |
| 22 | Exemplar | Hs.112405.0 | NM_002965.2 | g9845520 | RefSeq | Hs.112405 | fulllength | 6280 |
| | sequence | | | | | | | |
| 135 | Exemplar | Hs.33476.0 | NM_022765.1 | g12232436 | RefSeq | Hs.33476 | fulllength | 64780 |
| | sequence | | | | | | | |
| 277 | Exemplar | Hs.10649.1 | AB035482.1 | g8272423 | GenBank | Hs.10649 | fulllength | 9473 |
| | sequence | | | | | | | |
| 179 | Exemplar | Hs.278597.0 | NM_014369.1 | g7657483 | RefSeq | Hs.278597 | fulllength | 26469 |
| | sequence | | | | | | | |
| 45 | Consensus | Hs.20183.0 | BF439522 | Hs.20183.0.A1 | GenBank | Hs.374538 | fulllength | 153339 |
| | sequence | | | | | | | |
| 185 | Exemplar | Hs.129751.0 | NM_014339.1 | g7657229 | RefSeq | Hs.129751 | fulllength | 23765 |
| | sequence | | | | | | | |
| 241 | Exemplar | Hs.156114.0 | NM_004648.1 | g4758977 | RefSeq | Hs.156114 | fulllength | 140885 |
| | sequence | | | | | | | |
| 38 | Consensus | Hs.279518.4 | AW001847 | Hs.279518.4 | GenBank | Hs.279518 | fulllength | 334 |
| | sequence | | | | | | | |
| 217 | Consensus | Hs.75544.1 | AA284532 | Hs.75544.1.S1 | GenBank | Hs.302766 | fulllength | 152007 |
| | sequence | | | | | | | |
| 231 | Exemplar | Hs.100194.0 | NM_001629.1 | g4502058 | RefSeq | Hs.100194 | fulllength | 241 |
| | sequence | | | | | | | |
| 110 | Consensus | Hs.4248.1 | AA481141 | Hs.4248.1.S1 | GenBank | Hs.4248 | fulllength | |
| | sequence | | | | | | | |
| 68 | Consensus | Hs.180549.0 | BE396735 | Hs.180549.0_RC | GenBank | Hs.180549 | fulllength | 125308 |
| | sequence | | | | | | | |
| 262 | Consensus | Hs.10844.0 | AA622495 | Hs.10844.0.A1 | GenBank | Hs.10844 | fulllength | 116844 |
| | sequence | | | | | | | |
| 261 | Exemplar | Hs.325495.0 | NM_003255.2 | g9257247 | RefSeq | Hs.6441 | fulllength | 7077 |
| | sequence | | | | | | | |
| 270 | Exemplar | Hs.75108.0 | NM_002939.1 | g4506564 | RefSeq | Hs.75108 | fulllength | 6050 |
| | sequence | | | | | | | |
| 133 | Consensus | Hs.287353.0 | AC004832 | Hs.287353.0.S1 | GenBank | | | |
| | sequence | | | | | | | |
| 188 | Exemplar | Hs.171545.0 | NM_004504.2 | g7262381 | RefSeq | Hs.171545 | fulllength | 3267 |
| | sequence | | | | | | | |
| 125 | Consensus | Hs.306413.0 | AU144000 | Hs.306413.0_RC | GenBank | Hs.306413 | | |
| | sequence | | | | | | | |
| 136 | Exemplar | g13661555 | AF27o513.1 | g13661555 | GenBank | Hs.270143 | fulllength | 84034 |
| | sequence | | | | | | | |
| 143 | Exemplar | Hs.279607.0 | NM_001750.5 | g5729759 | RefSeq | Hs.359682 | fulllength | 831 |
| | sequence | | | | | | | |
| 159 | Consensus | Hs.48433.0 | AW613549 | Hs.48433.0.S1 | GenBank | Hs.48433 | fulllength | 24149 |
| | sequence | | | | | | | |
| 13 | Consensus | Hs.279518.1 | BC000373.1 | Hs.279518.1 | GenBank | Hs.279518 | fulllength | 334 |
| | sequence | | | | | | | |
| 23 | Exemplar | Hs.200333.0 | NM_018690.1 | g8922078 | RefSeq | Hs.200333 | fulllength | 55911 |
| | sequence | | | | | | | |
| 298 | Consensus | Hs.33033.0 | AI264121 | Hs.33033.0.A1 | GenBank | Hs.33033 | fulllength | 84898 |
| | sequence | | | | | | | |
| 236 | Exemplar | Hs.246381.0 | NM_001251.1 | g4557434 | RefSeq | Hs.246381 | fulllength | 968 |
| | sequence | | | | | | | |
| 1 | Exemplar | Hs.78575.0 | NM_002778.1 | g11386146 | RefSeq | Hs.406455 | fulllength | 5660 |
| | sequence | | | | | | | |
| 233 | Consensus | Hs.233276.0 | BE620374 | Hs.233276.0.S1 | GenBank | Hs.405915 | est | |
| | sequence | | | | | | | |
| 178 | Consensus | Hs.3446.0 | AI571419 | Hs.3446.0.A2 | GenBank | Hs.3446 | fulllength | 5604 |
| | sequence | | | | | | | |
| 119 | Exemplar | Hs.270737.0 | AF134715.1 | g7330729 | GenBank | Hs.270737 | fulllength | 10673 |
| | sequence | | | | | | | |
| 283 | Exemplar | Hs.43068.0 | AF251295.1 | g12005731 | GenBank | Hs.40368 | fulllength | 8905 |
| | sequence | | | | | | | |
| 147 | Exemplar | Hs.167679.2 | AB000463.1 | g1843393 | GenBank | Hs.167679 | fulllength | 6452 |
| | sequence | | | | | | | |
| 247 | Consensus | Hs.6823.0 | AI335263 | Hs.6823.0 | GenBank | Hs.6823 | fulllength | 81831 |
| | sequence | | | | | | | |
| 184 | Exemplar | Hs.202.1 | NM_000714.2 | g6382068 | RefSeq | Hs.202 | fulllength | 706 |
| | sequence | | | | | | | |
| 6 | Exemplar | Hs.279518.2 | BC004371.1 | g13325115 | GenBank | Hs.279518 | fulllength | 334 |
| | sequence | | | | | | | |
| 128 | Exemplar | Hs.75922.0 | AB055977.1 | g13094234 | GenBank | Hs.75922 | fulllength | 25798 |
| | sequence | | | | | | | |
| 171 | Consensus | Hs.11759.1 | BG497776 | Hs.11759.1.A1 | GenBank | Hs.11759 | fulllength | |
| | sequence | | | | | | | |
| 149 | Consensus | Hs.181156.0 | AI286239 | Hs.181156.0_RC | GenBank | Hs.181158 | est | |
| | sequence | | | | | | | |
| 86 | Exemplar | Hs.41726.0 | NM_002640.1 | g4505790 | RefSeq | Hs.41726 | fulllength | 5271 |
| | sequence | | | | | | | |
| 248 | Exemplar | Hs.71475.0 | NM_016630.2 | g13699804 | RefSeq | Hs.71475 | fulllength | 51324 |
| | sequence | | | | | | | |
| 249 | Consensus | Hs.102647.0 | AI934347 | Hs.102647.0.A1 | GenBank | Hs.102647 | | |
| | sequence | | | | | | | |
| 51 | Consensus | Hs.26506.0 | AI348000 | Hs.26506.0 | GenBank | Hs.26506 | fulllength | 92799 |
| | sequence | | | | | | | |
| 203 | Consensus | Hs.106876.0 | AL566172 | Hs.106876.0_RC | GenBank | Hs.106876 | fulllength | 9114 |
| | sequence | | | | | | | |
| 244 | Exemplar | Hs.226133.0 | NM_005890.1 | g5360211 | RefSeq | Hs.226133 | fulllength | 8522 |
| | sequence | | | | | | | |
| 126 | Exemplar | Hs.2621.0 | NM_005213.1 | g4885164 | RefSeq | Hs.412999 | fulllength | 1475 |
| | sequence | | | | | | | |
| 276 | Exemplar | Hs.118126.0 | NM_000308.1 | g450588 | RefSeq | Hs.118126 | fulllength | 5476 |
| | sequence | | | | | | | |
| 288 | Consensus | Hs.88411.7 | NM_007161.1 | Hs.88411.7 | GenBank | Hs.380427 | fulllength | 7940 |
| | sequence | | | | | | | |
| 205 | Consensus | Hs.146912.0 | AI990349 | Hs.146912.0_RC | GenBank | Hs.177376 | est | |
| | sequence | | | | | | | |
| 75 | Exemplar | g13543535 | BC005926.1 | g13543535 | GenBank | Hs.5509 | fulllength | 2124 |
| | sequence | | | | | | | |
| 55 | Exemplar | Hs.119529.0 | NM_006432.1 | g5453677 | RefSeq | Hs.433222 | fulllength | 10577 |
| | sequence | | | | | | | |
| 27 | Consensus | Hs.20478.0 | BG231932 | Hs.20478.0_RC | GenBank | Hs.20478 | fulllength | 1200 |
| | sequence | | | | | | | |
| 117 | Exemplar | Hs.80905.0 | NM_014737.1 | g7661963 | RefSeq | Hs.80905 | fulllength | 9770 |
| | sequence | | | | | | | |
| 93 | Exemplar | Hs.695.0 | NM_000100.1 | g4503116 | RefSeq | Hs.695 | fulllength | 1476 |
| | sequence | | | | | | | |
| 24 | Exemplar | Hs.11538.0 | NM_005720.1 | g5031600 | RefSeq | Hs.433506 | fulllength | 10095 |
| | sequence | | | | | | | |
| 56 | Consensus | Hs.99029.0 | AL564683 | Hs.99029.0 | GenBank | Hs.99029 | fulllength | 1051 |
| | sequence | | | | | | | |
| 232 | Consensus | Hs.110309.1 | AW514210 | Hs.110309.1 | GenBank | Hs.110309 | fulllength | 3134 |
| | sequence | | | | | | | |
| 26 | Exemplar | Hs.226133.2 | BC001152.1 | g12654630 | GenBank | Hs.226133 | fulllength | 8522 |
| | sequence | | | | | | | |
| 226 | Consensus | Hs.80887.0 | AI356412 | Hs.80887.0_RC | GenBank | Hs.80887 | fulllength | 4067 |
| | sequence | | | | | | | |
| 240 | Consensus | Hs.19980.0 | NM_019896.1 | Hs.19980.0.S1 | GenBank | Hs.19980 | fulllength | 56655 |
| | sequence | | | | | | | |
| 165 | Exemplar | Hs.76364.1 | U19713.1 | g1122908 | GenBank | Hs.76364 | fulllength | 199 |
| | sequence | | | | | | | |
| 160 | Exemplar | Hs.75811.1 | U47674.1 | g3860239 | GenBank | Hs.75811 | fulllength | 427 |
| | sequence | | | | | | | |
| 234 | Exemplar | Hs.101514.0 | BC000764.1 | g12653940 | GenBank | Hs.101514 | fulllength | 55122 |
| | sequence | | | | | | | |
| 7 | Exemplar | Hs.181013.0 | NM_002629.1 | g4505752 | RefSeq | Hs.181013 | fulllength | 5223 |
| | sequence | | | | | | | |
| 292 | Exemplar | Hs.14601.0 | NM_005335.1 | g4885404 | RefSeq | Hs.14601 | fulllength | 3059 |
| | sequence | | | | | | | |
| 141 | Exemplar | Hs.75873.0 | NM_003461.1 | g4508046 | RefSeq | Hs.75873 | fulllength | 7791 |
| | sequence | | | | | | | |
| 213 | Exemplar | Hs.25674.1 | AF072242.1 | g3800792 | GenBank | Hs.25674 | fulllength | 8932 |
| | sequence | | | | | | | |
| 157 | Consensus | Hs.28980.0 | AI674647 | Hs.28980.0.A1 | GenBank | Hs.28980 | fulllength | 84888 |
| | sequence | | | | | | | |
| 172 | Consensus | Hs.238954.0 | AW575123 | Hs.238954.0.A1 | GenBank | Hs.238954 | | 257106 |
| | sequence | | | | | | | |
| 5 | Exemplar | Hs.76506.0 | J02923.1 | g189501 | GenBank | Hs.381099 | fulllength | 3936 |
| | sequence | | | | | | | |
| 252 | Exemplar | Hs.6354.0 | NM_017455.1 | g9257239 | RefSeq | Hs.389371 | fulllength | 27020 |
| | sequence | | | | | | | |
| 297 | Exemplar | Hs.95821.0 | NM_0012383.1 | g6912563 | RefSeq | Hs.95821 | fulllength | 26578 |
| | sequence | | | | | | | |
| 260 | Exemplar | Hs.56319.0 | NM_001899.1 | g4503108 | RefSeq | Hs.56319 | fulllength | 1472 |
| | sequence | | | | | | | |
| 91 | Exemplar | Hs.279518.1 | BC000373.1 | g12653210 | GenBank | Hs.279518 | fulllength | 334 |
| | sequence | | | | | | | |
| 223 | Exemplar | Hs.103378.0 | AL136885.1 | g12053268 | GenBank | Hs.103378 | fulllength | 83641 |
| | sequence | | | | | | | |
| 60 | Exemplar | Hs.279518.0 | NM_001642.1 | g4502146 | RefSeq | Hs.279518 | fulllength | 334 |
| | sequence | | | | | | | |
| 99 | Exemplar | Hs.82212.0 | NM_000560.1 | g10834971 | RefSeq | Hs.82212 | fulllength | 63940 |
| | sequence | | | | | | | |
| 107 | Exemplar | Hs.288316.0 | NM_022107.1 | g11545816 | RefSeq | Hs.288316 | fulllength | 63940 |
| | sequence | | | | | | | |
| 114 | Exemplar | Hs.26367.1 | AF202092.1 | g11493699 | GenBank | Hs.26367 | fulllength | 64422 |
| | sequence | | | | | | | |
| 214 | Consensus | 4869209_rc | AJ243797 | 4869209_rc | GenBank | Hs.278408 | fulllength | 11277 |
| | sequence | | | | | | | |
| 279 | Exemplar | Hs.78944.0 | NM_002923.1 | g4506516 | RefSeq | Hs.78944 | fulllength | 5997 |
| | sequence | | | | | | | |
| 176 | Exemplar | Hs.279854.0 | U68567.1 | g1658373 | GenBank | Hs.279854 | fulllength | 4125 |
| | sequence | | | | | | | |
| 257 | Consensus | Hs.152932.0 | AI341602 | Hs.152932.0.A1 | GenBank | Hs.446474 | est | |
| | sequence | | | | | | | |
| 183 | Exemplar | Hs.6453.1 | AF279372.1 | g12006345 | GenBank | Hs.6453 | fulllength | 3705 |
| | sequence | | | | | | | |
| 216 | Exemplar | Hs.77422.0 | NM_002668.1 | g4505892 | RefSeq | Hs.77422 | fulllength | 5355 |
| | sequence | | | | | | | |
| 85 | Exemplar | Hs.3873.0 | NM_000310.1 | g4506030 | RefSeq | Hs.3873 | fulllength | 5538 |
| | sequence | | | | | | | |
| 158 | Consensus | Hs.10949.0 | BE966746 | Hs.10949.0.S1 | GenBank | Hs.10949 | | |
| | sequence | | | | | | | |
| 49 | Consensus | Hs.79356.0 | AI589086 | Hs.79356.0 | GenBank | Hs.79356 | fulllength | 7805 |
| | sequence | | | | | | | |
| 253 | Exemplar | g13543437 | BC005876.1 | g13543437 | GenBank | Hs.7476 | fulllength | 533 |
| | sequence | | | | | | | |
| 289 | Exemplar | Hs.78890.1 | AF015040.1 | g4102704 | GenBank | Hs.78890 | fulllength | 8650 |
| | sequence | | | | | | | |
| 47 | Exemplar | Hs.183434.0 | NM_005765.1 | g5031590 | RefSeq | Hs.183434 | fulllength | 10159 |
| | sequence | | | | | | | |
| 204 | Consensus | Hs.146550.0 | AI827941 | Hs.146550.0.A2 | GenBank | Hs.146550 | fulllength | 4627 |
| | sequence | | | | | | | |
| 134 | Consensus | Hs.155546.0 | NM_015044.1 | Hs.155546.0 | GenBank | Hs.155546 | fulllength | 23062 |
| | sequence | | | | | | | |
| 170 | Exemplar | Hs.75082.0 | NM_001665.1 | g4502218 | RefSeq | Hs.75082 | fulllength | 391 |
| | sequence | | | | | | | |
| 103 | Exemplar | Hs.94695.0 | AF208232.1 | g11493981 | GenBank | Hs.94695 | fulllength | 83982 |
| | sequence | | | | | | | |
| 113 | Consensus | Hs.75811.3 | AI934569 | Hs.75811.3.S1 | GenBank | Hs.75811 | fulllength | 427 |
| | sequence | | | | | | | |
| 230 | Exemplar | Hs.19122.0 | AF047695.1 | g3172338 | GenBank | Hs.19122 | fulllength | 9470 |
| | sequence | | | | | | | |
| 32 | Exemplar | Hs.20478.0 | NM_000391.2 | g5597012 | RefSeq | Hs.20478 | fulllength | 1200 |
| | sequence | | | | | | | |
| 104 | Exemplar | Hs.154023.0 | BC005374.1 | g13529223 | GenBank | Hs.154023 | fulllength | 23071 |
| | sequence | | | | | | | |
| 120 | Exemplar | Hs.104125.0 | NM_006367.2 | g10938021 | RefSeq | Hs.104125 | fulllength | 10487 |
| | sequence | | | | | | | |
| 272 | Exemplar | Hs.183868.0 | NM_000181.1 | g4504222 | RefSeq | Hs.183868 | fulllength | 2990 |
| | sequence | | | | | | | |
| 39 | Exemplar | Hs.6895.0 | AF004561.1 | g2209346 | GenBank | Hs.293750 | fulllength | 10094 |
| | sequence | | | | | | | |
| 148 | Consensus | Hs.278586.1 | AB029031.1 | Hs.278586.1_RC | GenBank | Hs.278586 | fulllength | 23216 |
| | sequence | | | | | | | |
| 144 | Consensus | Hs.85301.3 | AK000095.1 | Hs.85301.3.S1 | GenBank | Hs.356550 | fulllength | |
| | sequence | | | | | | | |
| 98 | Consensus | Hs.104125.2 | AA806142 | Hs.104125.2_RC | GenBank | Hs.104125 | fulllength | 10487 |
| | sequence | | | | | | | |
| 281 | Consensus | Hs.125078.2 | AF090094.1 | Hs.125078.2 | GenBank | Hs.405050 | | |
| | sequence | | | | | | | |
| 287 | Exemplar | Hs.75655.0 | J02783.1 | g339646 | GenBank | Hs.410578 | fulllength | 5034 |
| | sequence | | | | | | | |
| 87 | Exemplar | Hs.83583.0 | NM_005731.1 | g5031598 | RefSeq | Hs.83583 | fulllength | 10109 |
| | sequence | | | | | | | |
| 274 | Exemplar | Hs.89657.0 | NM_006284.1 | g5454105 | RefSeq | Hs.89657 | fulllength | 6881 |
| | sequence | | | | | | | |
| 219 | Consensus | Hs.9408.3 | BF213270 | Hs.9408.3 | GenBank | Hs.9408 | fulllength | 124944 |
| | sequence | | | | | | | |
| 189 | Exemplar | Hs.15977.0 | AF261090.1 | g9802311 | GenBank | Hs.15977 | fulllength | 4715 |
| | sequence | | | | | | | |
| 238 | Exemplar | Hs.7719.0 | NM_007278.1 | g6005763 | RefSeq | Hs.7719 | fulllength | 11337 |
| | sequence | | | | | | | |
| 207 | Exemplar | Hs.75103.0 | NM_003406.1 | g4507952 | RefSeq | Hs.75103 | fulllength | 7534 |
| | sequence | | | | | | | |
| 215 | Consensus | 4859973_rc | AA886893 | 4859973_rc | GenBank | Hs.8575 | fulllength | 54920 |
| | sequence | | | | | | | |
| 191 | Exemplar | Hs.1908.0 | J03223.1 | g190419 | GenBank | Hs.1908 | fulllength | 5552 |
| | sequence | | | | | | | |
| 221 | Exemplar | Hs.9534.0 | NM_014300.1 | g7657608 | RefSeq | Hs.9534 | fulllength | 23478 |
| | sequence | | | | | | | |
| 291 | Exemplar | Hs.182278.1 | BC000454.1 | g12653368 | GenBank | Hs.282410 | fulllength | 801 |
| | sequence | | | | | | | |
| 29 | Exemplar | Hs.5322.0 | NM_005274.1 | g4885286 | RefSeq | Hs.424138 | fulllength | 2787 |
| | sequence | | | | | | | |
| 123 | Consensus | Hs.301175.1 | B138888 | Hs.301175.1 | GenBank | Hs.301175 | fulllength | 5880 |
| | sequence | | | | | | | |
| 118 | Consensus | Hs.252280.1 | BG034239 | Hs.252280.1_RC | GenBank | Hs.83583 | fulllength | 10109 |
| | sequence | | | | | | | |
| 192 | Exemplar | Hs.184062.2 | BC004446.1 | g13325254 | GenBank | Hs.184062 | fulllength | 55969 |
| | sequence | | | | | | | |
| 222 | Consensus | Hs.44499.2 | BE568651 | Hs.44499.2_RC | GenBank | Hs.44499 | fulllength | 5411 |
| | sequence | | | | | | | |
| 109 | Exemplar | Hs.78771.0 | NM_000291.1 | g4505762 | RefSeq | Hs.78771 | fulllength | 5230 |
| | sequence | | | | | | | |
| 199 | Exemplar | Hs.184062.0 | NM_018840.1 | g10047115 | RefSeq | Hs.184062 | fulllength | 55969 |
| | sequence | | | | | | | |
| 254 | Exemplar | Hs.289101.0 | D83485.1 | g1208426 | GenBank | Hs.13751 | fulllength | 2923 |
| | sequence | | | | | | | |
| 194 | Exemplar | Hs.76293.0 | NM_021103.1 | g10863894 | RefSeq | Hs.76293 | fulllength | 9168 |
| | sequence | | | | | | | |
| 268 | Consensus | Hs.113205.2 | AA854966 | Hs.113205.S1 | GenBank | Hs.433419 | fulllength | 1327 |
| | sequence | | | | | | | |

| | Full |
|---|---|
| | Length |
| | Reference |
| Number | Seq |
| 239 | NM_006864; leukocyte immunoglobulin-like receptor, |
| | subfamily B (with TM and ITIM domains), member 3 |
| 278 | NM_006864; leukocyte immunoglobulin-like receptor, |
| | subfamily B (with TM and ITIM domains), member 3 |
| 132 | NM_006864; leukocyte immunoglobulin-like receptor, |
| | subfamily B (with TM and ITIM domains), member 3 |
| 275 | NM_004385; chondroitin sulfate proteoglycan 2 (versican) |
| 96 | NM_002115; hexokinase 3 |
| 89 | |
| 264 | NM_001766; CD1D antigen, d polypeptide |
| 210 | NM_021642; Fc fragment of IgG, low affinity tla, receptor for (CD32) |
| 83 | NM_004235; Kruppel-like factor 4 (gut) |
| 299 | NM_006864; leukocyte immunoglobulin-like receptor, |
| | subfamily B (with TM and ITIM domains), member 3 |
| 19 | |
| 35 | NM_004390; cathepsin H isoform a preproprotein NM_148979; |
| | cathepsin H isoform b precursor |
| 211 | NM_006065; signal-regulatory protein beta 1 precursor |
| 259 | NM_013439; paired immunoglobulin-like receptor alpha isoform 1 |
| | precursor NM_178272; paired immunoglobulin-like receptor alpha isoform 2 |
| | precursor NM_178273; paired immunoglobulin-like receptor alpha isoform 3 precursor |
| 21 | NM_006332; interferon, gamma-inducible protein 30 preproprotein |
| 54 | NM_013439; paired immunoglobulin-like receptor alpha isoform 1 |
| | precursor NM_178272; paired immunoglobulin-like receptor alpha isoform 2 |
| | precursor NM_178273; paired immunoglobulin-like receptor alpha isoform 3 precursor |
| 76 | NM_005461; trasncription factor MAFB NM_152258; |
| 69 | NM_000295; serine (or cysteine) proteinase inhibitor, clade A |
| | (alpha-1 antiproteinase, antitrypsin), member 1 |
| 267 | NM_138444; hypothetical protein BC013764 |
| 100 | NM_000507; fructose-1,6-bisphosphatase 1 |
| 202 | NM_006889; CD86 antigen isoform 2 precursor NM_175862; |
| | CD86 antigen isoform 1 |
| 44 | NM_000952; platelet-activating factor receptor |
| 224 | NM_005615; ribonuclease, RNAse A family, k6 |
| 31 | NM_004385; chondroitin sulfate proteoglycan 2 (versican) |
| 152 | NM_012072; complement component 1, q subcomponent, receptor 1 |
| 250 | NM_000265; neutrophil cytosolic factor 1 |
| 25 | NM_004385; chondroitin sulfate proteoglycan 2 (versican) |
| 48 | NM_005461; transcription factor MAFB NM_152258; |
| 18 | NM_021626; serine carboxypeptidase 1 precursor protein |
| 101 | NM_002621; properdin P factor, complement |
| 256 | NM_001736; complement component 5 receptor 1 (C5a ligand) |
| 11 | NM_001154; annexin 5 |
| 70 | NM_000351; steroid sulfatase (microsomal), arylsulfatase C, isozyme S |
| 34 | NM_019027; RNA-binding protein |
| 228 | NM_002003; ficolin 1 precursor |
| 284 | NM_006889; CD86 antigen isoform 2 precursor |
| | NM_175862; CD86 antigen isoform 1 |
| 137 | NM_022162; NOD2 protein |
| 42 | NM_000433; neutrophil cytosolic factor 2 |
| 12 | NM_004106; Fc fragment of IgE, high affinity I, receptor for, |
| | gamma polypeptide precursor |
| 4 | NM_005248; Gardner-Rasheed faline sarcoma viral (v-fgr) oncogene homolog |
| 294 | NM_174902; hypothetical protein LOC143458 |
| 167 | NM_000442; platelethendothelial cell adhesion molecule (CD31 antigen) |
| 46 | NM_017424; cat eye symdrome critical region protein 1 |
| | isoform a precursor NM_177405; cat eye syndrome critical region |
| | protein 1 isoform b |
| 130 | NM_000952; platelet-activating factor receptor |
| 72 | NM_006866; leukocyte immunoglobulin-like receptor, subfamily A (with TM domain), member 2 |
| 79 | NM_15474; SAM domain and HD domain 1 |
| 121 | NM_006176; neurogranin |
| 94 | NM_022349; membrane-spanning 4-domains, subfamily A, |
| | member 6A isoform 2 NM_152851; membrane-spanning 4-domains |
| | subfamily A, member 6A isoform 3 NM_152852: membrane-spanning 4-domains, |
| | subfamily A, member 6A isoform 1 |
| 102 | NM_019027; RNA-binding protein |
| 15 | NM_002087; granulin |
| 90 | NM_022349; membrane-spanning 4-domains, subfamily A, |
| | member 6A isoform 2 NM_152851; membrane-spanning 4-domains, |
| | subfamily A, member 6A isoform 3 NM_152852; membrane-spanning |
| | 4-domains, subfamily A, member 6A isoform 1 |
| 105 | |
| 220 | NM_006866; leucocyte immunoglobulin-like receptor, subfamily A |
| | (with TM domain), member 2 |
| 57 | NM_002778; prosaposin (variant Gaucher disease and variant |
| | metachromatic leukodystrophy) |
| 80 | NM_006678; CMRF35 leukocyte immunoglobulin-like receptor |
| 64 | NM_004079; cathepsin S preproprotein |
| 106 | NM_004271; MD-1, RP105-associated |
| 95 | NM_006866; leucocyte immunoglobulin-like receptor, |
| | subfamily A (with TM domain), member 2 |
| 9 | NM_002087; granulin |
| 82 | |
| 243 | NM_018837; similar to glucosamine-6-sulfatases |
| 145 | NM_002076; glucosamine (N-acetyl)-6-sulfatase precursor |
| 65 | NM_020350; angiotensin II receptor-associated protein |
| 166 | NM_006449; Cdc42 effector protein 3 |
| 88 | NM_002076; glucosamine (N-acetyl)-6-sulfatase precursor |
| 229 | NM_000389; cyclin-dependent kinase inhibitor 1A NM_078467; |
| | cyclin-dependent kinase inhibitor 1A |
| 290 | NM_021205; ras homolog gene family, member U |
| 162 | NM_024318; leukocyte immunoglobulin-like receptor, subfamily |
| | B (with TM and ITIM domains), member 6 |
| 263 | NM_012072; complement component 1, q subcomponent, receptor 1 |
| 33 | NM_000218; potassium voltage-gated channel, |
| | KQT-like subfamily precursor, member 1 |
| 282 | |
| 177 | NM_001637; acyloxyacyl hydrolase precursor |
| 175 | NM_001908; cathepsin B preproprotein NM_147760; cathepsin |
| | B preproprotein NM_147781; cathepsin B preproprotein NM_147782; |
| | cathepsin B preproprotein NM_147783; cathepsin B preproprotein |
| 174 | NM_002306; lectin, galactoside-binding, soluble, 3 (galactin 3) |
| 255 | NM_022349; membrane-spanning 4-domains, subfamily A, member 6A |
| | isoform 2 NM_152851; membrane-spanning 4-domains, subfamily A, |
| | member 6A isoform 3 NM_152852; membrane-spanning 4-domains, |
| | subfamily A, member 6A isoform 1 |
| 14 | NM_002087; granulin |
| 30 | NM_021149; coactosin-like 1 |
| 251 | NM_000887; integrin alpha X precursor |
| 112 | NM_00042; platelet/endothelial cell adhesion molecule (CD31 antigen) |
| 286 | |
| 17 | NM_000099; cystatin C precursor |
| 84 | NM_174896; hypothetical protein MGC24133 |
| 3 | |
| 28 | NM_005195; CCAAT/enhancer binding protein delta |
| 265 | |
| 246 | NM_003264; toll-like receptor 2 |
| 92 | NM_003644; growth arrest-specific 7 isoform a NM_005890; |
| | growth arrest-specific 7 isoform b |
| 108 | NM_002076; glucosamine (N-acetyl)-8-sulfatase precursor |
| 111 | NM_014624; S100 calcium-binding protein A6 |
| 295 | |
| 73 | NM_007162; transcription factor E8 |
| 156 | NM_000376; vitamin D (1.25-dihydroxyvitamin D3) receptor |
| 36 | NM_004648; NM_080792; protein tyrosine phosphatase, |
| | non-receptor type substrate 1 precursor |
| 8 | NM_004039; annexin A2 |
| 66 | NM_021149; coactosin-like 1 NM_021615; |
| | carbohydrate (N-acetylglucosamine 6-O) sulfotransferase 6 |
| 77 | |
| 10 | NM_004039; annexin A2 |
| 41 | NM_002966; S100 calcium-binding protein A10 |
| 164 | NM_001560; interleukin 13 receptor, alpha 1 precursor |
| 20 | NM_000521; hexosaminidase B preproprotein |
| 146 | NM_003916; adaptor-related protein complex 1 sigma 2 subunit |
| 197 | |
| 37 | NM_000405; GM2 ganglloside activator protein precursor |
| 16 | NM_004039; annexin A2 |
| 155 | NM_002959; sortilin 1 preproprotein |
| 227 | |
| 273 | NM_001706: B-cell lymphoma 6 protein NM_138931: |
| | B-cell lymphoma 6 protein |
| 61 | |
| | NM_002163; interferon consensus sequence binding protein 1 |
| 200 | |
| 40 | NM_003332; TYRO protein tyrosine kinase binding protein |
| 293 | NM_000351; steroid sulfatase (microsomal), arylsulfatase C, isozyme S |
| 116 | |
| 52 | NM_003916; adaptor-related protein complex 1 sigma 2 subunit |
| 81 | |
| 181 | NM_006317; brain abundant, membrane attached signal proptein 1 |
| 78 | NM_003644; growth arrest-specifuc 7 isoform a NM_005890. |
| | growth arrest-specific 7 isiform b |
| 122 | NM_002305; beta-galactosidase binding lectin percusor |
| 201 | NM_003407; zinc finger protein 36, C3H type, homolog |
| 245 | NM_005534; interfaron gamma receptor 2 (interferon gamma transduce 1) |
| 169 | NM_000628; interleukin 10 receptor, beta precursor |
| 187 | NM_000442; platelet/endothelial cell adhesion molecule (CD31 antigen) |
| 43 | NM_001748; capain 2, large subunit |
| 173 | NM_022003; FXYD domain-containing ion transport regulator 6 |
| 198 | NM_004313; arrestin beta 2 |
| 266 | NM_022343; chromosome 9 open reding frame 19 |
| 168 | NM_014210; ecotropic viral integration site 2A |
| 237 | NM_002957; retinoid X receptor, alpha |
| 153 | |
| 50 | NM_006019; T-cell, immune regulator 1, isoform a NM_006053; |
| | T-cell, immune regulator 1, isoform b |
| 59 | NM_001908; cathepsin B preproprotein NM_147780; cathepsin |
| | B preprotein NM_147781; cathapsin B preproprotein NM_147782; |
| | cathepsin B preproprotein NM_147783; cathepsin B preproprotein |
| 71 | NM_004079; cathepsin S preproprotein |
| 53 | NM_000405; GM2 ganglioside activator protein precursor |
| 193 | NM_001750; caloastain isoform a NM_173060; caloestain |
| | isoform b NM_173061; calpastatin isoform c NM_173062; calpastain isoform d |
| 182 | |
| 58 | NM_002961; S100 calcium-binding protein A4 NM_019554; |
| | S100 calcium-binding protein A4 |
| 2 | NM_000211; integrin beta chain, beta 2 precursor |
| 97 | NM_001706; B-cell lymphoma 6 protein NM_138931; |
| | B-cell lymphoma 6 protein |
| 67 | NM_001560; interleukin 13 receptor, alpha 1 precursor |
| 115 | NM_000632; integrin alpha M precursor |
| 280 | NM_003808; tumor necrosis factor ligand superfamily, member |
| | 13 isoform alpha precursor NM_172088; tumor necrosis factor ligand |
| | superfamily, member 13 isoform beta NM_172088; tumor nercrosis factor |
| | ligand superfamily, member 13 isoform gamma NM_172089; |
| | tumor necrosis factor ligand superfamily, member 13 isoform delta |
| 22 | NM_002965; S100 calcium-binding protein A9 |
| 135 | NM_022765; NEDD9 interacting protein with calponin homology and LIM dimains |
| 277 | NM_004848; basement membrane−induced gene |
| 179 | NM_014369; protein tyrosine phosphatase, non-receptor type 18 |
| 45 | NM_174909; hypothetical protein LOC153339 |
| 185 | NM_014339; interleukin 17 receptor precursor |
| 241 | NM_004648; NM_080792: protein tyrosine phosphatase, |
| | non-receptor type substrate 1 precursor |
| 38 | NM_001642; amyloid beta (A4) precursor-like protein 2 NM_016160; |
| 217 | NM_022343; chromosome 9 open reading frame 19 |
| 231 | NM_001629; arachidonate 5-lipoxygenasE−activating protein |
| 110 | |
| 68 | NM_130807; MOB-LAK |
| 262 | NM_052972; leucine−rich alpha-2-glycoprotein |
| 261 | NM_003255; tissue inhibitor of metalloproteinase 2 precursor |
| 270 | NM_002939; ribonuclease/angiogenin inhibitor |
| 133 | |
| 188 | NM_004504; HIV-1 Rav binding protein |
| 125 | |
| 136 | NM_032048; extracellular glycoprotein EMILIN-2 precursor |
| 143 | NM_001750; calpastatin isoform a NM_173060; |
| | calpastatin isoform b NM_173061; |
| | calpastatin isoform c NM_173062; calpastatin isoform d |
| 159 | NM_014345; zinc finger protein 316 |
| 13 | NM_001642; amyloid beta (A4) precursor-like protein 2 NM_016160; |
| 23 | NM_018690; apollpoprotein B48 receptor |
| 298 | NM_032812; tumor endothelial marker 7-related precursor |
| 236 | NM_001251; CD68 antigen |
| 1 | NM_002778; prosapoain (variant Gaucher disesase and variant |
| | metachromatic leukodystrophy) |
| 233 | |
| 178 | NM_002755; mitogen-activated protein kinase 1 |
| 119 | NM_006573; tumor necrosis factor (ligand) superfamily, member 13b |
| 283 | NM_003916; adaptor-related protein complex 1 sigma 2 subunit |
| 147 | NM_003023; SH3-domain binding protein 2 |
| 247 | NM_018092; neuropilin- and tolloid-like protein 2 precursor |
| 184 | NM_000714; peripheral benzodiazapine receptor NM_007311; |
| | peripheral benzodiazapine receptor short form |
| 6 | NM_001642; amyloid beta (A4) precursor-like protein 2 NM_016160; |
| 128 | NM_015379; brain protein I3 |
| 171 | |
| 149 | |
| 86 | NM_002640; serine (or cysteine) proteinase inhibitor, clade B (ovalbumin), member B |
| 248 | NM_016630; acid cluster protein 33 |
| 249 | |
| 51 | NM_138392; hypothetical protein BC007653 |
| 203 | NM_004691; ATPase, H+ transporting, lysosomal, V0 subunit D, isoform 1 |
| 244 | NM_003644; growth arrest-specific 7 isoform a NM_005890; |
| | growth arrest-specific 7 isoform b |
| 126 | NM_005213; cystatin A |
| 276 | NM_000308; protective protein for beta-galactosidase |
| 288 | NM_007161; leukocyte specific transcript 1 |
| 205 | |
| 75 | NM_006495; ecotropic viral integration site 2B |
| 55 | NM_006432; Niemann-Pick disease, type C2 precursor |
| 27 | NM_000391; ceroid-lipofuscinosis, neuronal 2, late infantile |
| | (Jansky-Bielschowsky disease) |
| 117 | NM_014737; Ras association domain family 2 isoform 1 NM_170773; |
| | Ras association domain family 2 isoform 2 NM_170774; |
| | Ras association domain family 2 isoform 1 |
| 93 | NM_000100; cystatin B |
| 24 | NM_005720; actin related protein 2/3 complex subunit 1B |
| 56 | NM_005194; CCAAT/enhancer binding protein beta |
| 232 | NM_018950; major histocompatibility complex, class I, F precursor |
| 26 | NM_003644; growth arrest-specific 7 isoform a NM_005890; |
| | growth arrest-specific 7 isoform b |
| 226 | NM_002350; v-yes-1 Yamaquchi sarcoma viral related oncogene homolog |
| 240 | NM_019896; polymerase (DNA-directed), epsilon 4 (p12 subunit) |
| 165 | NM_001623; allograft inflammatory factor 1 isoform 3 NM_004847; |
| | allograft inflammatory factor 1 isoform 2 NM_032955; |
| | allograft inflammatory factor 1 isoform 1 |
| 160 | NM_004315; N-acylsphingosine amidohydrolase (acid caramidase) 1 |
| | isoform b NM_177924; N-acylsphingosine amidohydrolase (acid carmidase) 1 |
| | preproprotein isoform a |
| 234 | NM_018064; hypothetical protein FLJ10342 |
| 7 | NM_002629; phosphoglycerate mutase 1 (brain) |
| 292 | NM_005335; hematopoletic cell-specific Lyn substrate 1 |
| 141 | NM_003461; zyxin |
| 213 | NM_003927; methyl-CpG binding domain protein 2 isoform 1 NM_015832; |
| | methyl-CpG binding domain protein 2 testis-specific isoform |
| 157 | NM_032802; putative intramembrane cleaving protease |
| 172 | |
| 5 | NM_002298; L-plastin |
| 252 | NM_012428; stromal cell derived factor receptor 1 isoform b NM_017455; |
| | stromal cell derived factor receptor 1 isoform a |
| 297 | NM_012383; osteoclast stimulating factor 1 |
| 260 | NM_001899; cystatin S precursor |
| 91 | NM_001642; amyloid beta (A4) precursor-like protein 2 NM_016160; |
| 223 | NM_031453; hypothetical protein MGC11034 |
| 60 | NM_001642; amyloid beta (A4) precursor-like protein 2 NM_016160; |
| 99 | NM_000560; CD53 antigen |
| 107 | NM_022107; chromosome 6 open reading frame 9 |
| 114 | NM_022488; Apg3p |
| 214 | NM_016381; three prime repair exonuclease 1 isoform a NM_032166; |
| | three prime repair exonuclease 1 isoform c NM_033627; three prime repair |
| | exonuclease 1 isoform b NM_033628; three prime repair exonuclease 1 |
| | isoform b NM_033629; three prime repair exonuclease 1 |
| | isoform b NM_130384; three prime repair exonuclease 1 isoform d |
| 279 | NM_002923; regulator of G-protein signalling 2, 24 kDa |
| 176 | NM_000528; mannosidase, alpha, class 2B, member 1 |
| 257 | |
| 183 | NM_014216; inositol 1,3,4-triphosphate 5/6 kinase |
| 216 | NM_002668; proteolipid protein 2 (colonic epithelium-enriched) |
| 85 | NM_000310; palmitoyl-protein thioesterase 1 |
| | (ceroid-lipofuscinosis, neuronal 1, infantile) |
| 158 | |
| 49 | NM_006762; Lysosomal-associated multispanning |
| | membrane protein-5 |
| 253 | NM_004047; ATPase, H+ transporting, lysosomal, 21 kD, V0 subunit c [symbol] |
| 289 | NM_003744; numb homolog |
| 47 | NM_005765; ATPase, H+ transporting, lysosomal, |
| | membrane sector associated protein M8-9 |
| 204 | NM_002473; myosin, heavy polypeptide 9, non-muscle |
| 134 | NM_015044; ADP-ribosylation factor binding protein 2 isoform 1 |
| | NM_138640; ADP-ribosylation factor binding protein 2 isoform 2 |
| 170 | NM_001665; ras homolog gene family, member G (rho G) |
| 103 | NM_032036; TLH29 protein precursor |
| 113 | NM_004315; N-acylsphingosine amidohydrolase (acid ceramidase) 1 |
| | isoform b NM_177924; N-acylsphingosine amidohydrolase (acid ceramidase) 1 |
| | preproprotein isoform a |
| 230 | NM_004846; eukaryotic translation initiation factor 4E-like 3 |
| 32 | NM_000391; ceroid-lipofuscinosis, neuronal 2, late infantile |
| | (Jansky-Bielschowsky disease) |
| 104 | |
| 120 | NM_006367; adenylyl cyclase-associated protein |
| 272 | NM_000181; glucuronidase, beta |
| 39 | NM_005719; actin related protein 2/3 complex subunit 3 |
| 148 | NM_015173; TBC1 (tre-2/USP6, BUB2, cdc16) domain family, member 1 |
| 144 | |
| 98 | NM_006367; adenylyl cyclase-associated protein |
| 281 | |
| 287 | NM_000918; prolyl 4-hydroxylase, beta subunit |
| 87 | NM_005731; actin related protein 2/3 complex subunit 2 NM_152862; |
| | actin related protein 2/3 complex subunit 2 |
| 274 | NM_006284; TBP-related factor 10 |
| 219 | |
| 189 | NM_005005; NADH dehydrogenase (ubiquinone) 1 beta subcomplex, 9, 22 kDa |
| 238 | NM_007278; GABA(A) receptor-associatd protein |
| 207 | NM_003406; tyrosine3/tryptophan 5-monooxygenase activation protein, |
| | zeta polypeptide NM_145690; tyrosine 3/tryptophan 5-monooxygenase |
| | activation protein, zeta polypeptide |
| 215 | NM_017803; hypothetical protein FLJ20399 |
| 191 | NM_002727; proteoglycan 1, secretory granule |
| 221 | NM_014300; signal peptidase complex (18 kD) |
| 291 | NM_006888; calmodulin 1 (phosphorylase kinase, delta) |
| 29 | NM_005274; guanine nucleotide binding protein (G protein), gamma 6 |
| 123 | NM_002872; ras-related C3 botulinum toxin substrate 2 NM_014029; |
| 118 | NM_005731; actin related protein 2/3 complex subunit 2 NM_152862; |
| | actin related protein 2/3 complex subunit 2 |
| 192 | NM_018840; putative Rab5-interacting protein |
| 222 | NM_002687; pinin, desmosome associated protein |
| 109 | NM_000291; phosphoglycerate kinase 1 |
| 199 | NM_018840; putative Rab5-interacting protein |
| 254 | NM_005313; glucose regulated protein, 58 kDa |
| 194 | NM_021103; thymosin, beta 10 |
| 268 | NM_001861; cytochrome c oxidase subunit IV isofom 1 precursor |

[symbol]

indicates data missing or illegible when filed

TABLE 41
Downregulated or lower expressed genes in cases with (del)5q compared to cases without (del)5q

| affy id | HUGO name | Title | MapLocation | Sequence Type | Go_Biological_Process | Go_Cellular_Component |
|---|---|---|---|---|---|---|
| 1 | 203454_s_at | ATOX1 | ATX1 antioxidant protein 1 homolog | 5q32 | Exemplarsequence | “GO: 6878; copper ion homeostasis; traceable author statement |
| | | | (yeast) | | | GO: 30001; metal ion transport; inferred from electronic |
| | | | | | | annotation GO: 6825; copper ion transport; traceable author |
| | | | | | | statement GO: 6979; response to oxidative stress; traceable |
| | | | | | | author statement” |
| 2 | 201310_s_at | C5orf13 | chromosome 5 open reading frame 13 | 5q22.1 | Exemplarsequence | |
| 3 | 201309_x_at | C5orf13 | chromosome 5 open reading frame 13 | 5q22.1 | Exemplarsequence | |
| 4 | 230424_at | C5orf13 | chromosome 5 open reading frame 13 | 5q22.1 | Consensussequence | |
| 5 | 48031_r_at | C5orf4 | chromosome 5 open reading frame 4 | 5q31-q32 | Consensussequence | |
| 6 | 48030_i_at | C5orf4 | chromosome 5 open reading frame 4 | 5q31-q32 | Consensussequence | |
| 7 | 220751_s_at | C5orf4 | chromosome 5 open reading frame 4 | 5q31-q32 | Exemplarsequence | |
| 8 | 218518_at | C5orf5 | chromosome 5 open reading frame 5 | 5q31 | Exemplarsequence | |
| 9 | 202164_s_at | CNOT8 | CCR4-NOT transcription complex, | 5q31-q33 | Exemplarsequence | “GO: 6355; regulation of transcription, DNA-dependent; non- |
| | | | subunit 8 | | | traceable author statement” |
| 10 | 202163_s_at | CNOT8 | CCR4-NOT transcription complex, | 5q31-q33 | Exemplarsequence | “GO: 6355; regulation of transcription, DNA-dependent; non- |
| | | | subunit 8 | | | traceable author statement” |
| 11 | 202162_s_at | CNOT8 | CCR4-NOT transcription complex, | 5q31-q33 | Consensussequence | “GO: 6355; regulation of transcription, DNA-dependent; non- |
| | | | subunit 8 | | | traceable author statement” |
| 12 | 217491_x_at | COX7C | cytochrome c oxidase subunit VIIc | 5q14 | Consensussequence | “GO: 6118; electron transport; inferred from electronic |
| | | | | | | annotation GO: 6091; energy pathways; traceable author |
| | | | | | | statement” |
| 13 | 213846_at | COX7C | cytochrome c oxidase subunit VIIc | 5q14 | Consensussequence | “GO: 6118; etectron transport; inferred from electronic |
| | | | | | | annotation GO: 6091; energy pathways; traceable author |
| | | | | | | statement” |
| 14 | 201134_x_at | COX7C | cytochrome c oxidase subunit VIIc | 5q14 | Exemplarsequence | “GO: 6118; electron transport; inferred from electronic |
| | | | | | | annotation GO: 6091; energy pathways; traceable author |
| | | | | | | statement” |
| 15 | 226920_at | CSNK1A1 | casein kinase 1, alpha 1 | 5q32 | Consensussequence | “GO: 7165; signal transduction; not recorded GO: 7166; cell |
| | | | | | | surface receptor linked signal transduction; experimental |
| | | | | | | evidence GO: 6468; protein amino acid |
| | | | | | | phosphorylation; experimental evidence” |
| 16 | 213086_s_at | CSNK1A1 | casein kinase 1, alpha 1 | 5q32 | Consensussequence | “GO: 7165; signal transduction; not recorded GO: 7166; cell |
| | | | | | | surface receptor linked signal transduction; esperimental |
| | | | | | | evidence GO: 6468; protein amino acid |
| | | | | | | phosphorylation; experimental evidence” |
| 17 | 208867_s_at | CSNK1A1 | casein kinase 1, alpha 1 | 5q32 | Exemplarsequence | “GO: 7165; signal transduction; not recorded GO: 7166; cell |
| | | | | | | surface receptor linked signal transduction; experimental |
| | | | | | | evidence GO: 6468; protein amino acid |
| | | | | | | phosphorylation; experimental evidence” |
| 18 | 208866_at | CSNK1A1 | casein kinase 1, alpha 1 | 5q32 | Consensussequence | “GO: 7165; signal transduction; not recorded GO: 7166; cell |
| | | | | | | surface receptor linked signal tranaduction; experimental |
| | | | | | | evidence GO: 6468; protein amino acid |
| | | | | | | phosphorylation; experimental |
| | | | | | | evidence” |
| 19 | 208865_at | CSNK1A1 | casein kinase 1, alpha 1 | 5q32 | Consensussequence | “GO: 7165; signal transduction; not recorded GO: 7166; cell |
| | | | | | | surface receptor linked signal transduction; experimental |
| | | | | | | evidence GO: 6468; protein amino acid |
| | | | | | | phosphorylation; experimental evidence” |
| 20 | 206562_s_at | CSNK1A1 | casein kinase 1, alpha 1 | 5q32 | Exemplarsequence | “GO: 7165; signal transduction: not recorded GO: 7166; cell |
| | | | | | | surface receptor linked signal transduction; experimental |
| | | | | | | evidence GO: 6468; protein amino acid |
| | | | | | | phosphorylation; experimental evidence” |
| 21 | 235464_at | CSNK1A1 | casein kinase 1, alpha 1 | 5q32 | Consensussequence | “GO: 7165; signal transduction; not recorded GO: 7166; cell |
| | | | | | | surface receptor linked signal transduction; experimental |
| | | | | | | evidence GO: 6468; protein amino acid |
| | | | | | | phosphorylation; experimental evidence” |
| 22 | 220263_at | DAMS | SMAD in the antisense orientation | 5q31.1 | Exemplarsequence | “GO: 7165; signal transduction; traceable author statement” |
| 23 | 222488_s_at | DCTN4 | dynactin 4 (p62) | 5q31-q32 | Consensussequence | |
| 24 | 218013_x_at | DCTN4 | dynactin 4 (p62) | 5q31-q32 | Exemplarsequence | |
| 25 | 233490_at | DCTN4 | dynactin 4 (p62) | 5q31-q32 | Consensussequence | |
| 26 | 222488_s_at | DCTN4 | dynactin 4 (p62) | 5q31-q32 | Consensussequence | |
| 27 | 218013_x_at | DCTN4 | dynactin 4 (p62) | 5q31-q32 | Exemplarsequence | |
| 28 | 233490_at | DCTN4 | dynactin 4 (p62) | 5q31-q32 | Consensussequence | |
| 29 | 233087_at | FBXO13 | F-box only protein 13 | 5q21.3 | Consensussequence | |
| 30 | 223833_at | FLJ20195 | hypothetical protein FLJ20195 | 5q31.3 | Exemplarsequence | |
| 31 | 57739_at | FLJ20195 | hypothetical protein FLJ20195 | 5q31.3 | Consensussequence | |
| 32 | 222114_x_at | FLJ20195 | hypothetical protein FLJ20195 | 5q31.3 | Consensussequence | |
| 33 | 222070_at | FLJ20195 | hypothetical protein FLJ20195 | 5q31.3 | Consensussequence | |
| 34 | 219809_at | FLJ20195 | hypothetical protein FLJ20195 | 5q31.3 | Exemplarsequence | |
| 35 | 241734_at | FLJ25286 | hypothetical protein FLJ25286 | 5q23.1 | Consensussequence | |
| 36 | 212256_at | GALNT10 | UDP-N-acetyl-alpha-D- | 5q33.2 | Consensussequence | |
| | | | galactosamine: polypeptide N- | | | |
| | | | acetytgalactosaminyltransferase 10 | | | |
| | | | (GalNAc-T10) | | | |
| 37 | 207357_s_at | GALNT10 | UDP-N-acetyl-alpha-D- | 5q33.2 | Exemplarsequence | |
| | | | galactosamine: polypeptide N- | | | |
| | | | acetylgalactosaminyltransferase 10 | | | |
| | | | (GalNAc-T10) | | | |
| 38 | 230906_at | GALNT10 | UDP-N-acetyl-alpha-D- | 5q33.2 | Consensussequence | |
| | | | galactosamine: polypeptide N- | | | |
| | | | acetylgalactosaminyltransferase 10 | | | |
| | | | (GalNAc-T10) | | | |
| 39 | 35820_at | GM2A | GM2 ganglioside activator protein | 5q31.3-q33.1 | Consensussequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 40 | 33646_g_at | GM2A | GM2 ganglioside activator protein | 5q31.3-q33.1 | Consensussequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 41 | 215891_s_at | GM2A | GM2 ganglioside activator protein | 5q31.3-q33.1 | Consensussequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 42 | 215890_at | GM2A | GM2 ganglioside activator protein | 5q31.3-q33.1 | Consensussequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 43 | 212737_at | GM2A | GM2 ganglioside activator protein | 5q31.3-q33.1 | Consensussequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 44 | 209727_at | GM2A | GM2 ganglioside activator protein | 5q31.3-q33.1 | Exemplarsequence | “GO: 6687; glycosphingolipid metabolism; inferred from |
| | | | | | | electronic annotation GO: 30149; sphingolipid catabolism; non- |
| | | | | | | traceable author statement GO: 19377; glycolipid |
| | | | | | | catabolism; non-traceable author statement” |
| 45 | 216326_s_at | HDAC3 | histone deacetylase 3 | 5q31 | Consensussequence | “GO: 74; regulation of cell cycle; traceabte author statement” |
| 46 | 202540_s_at | HMGCR | 3-hydroxy-3-methylglutaryl-Coenzyme A | 5q13.3-q14 | Exemplarsequence | “GO: 8406; gonad development; traceable author statement |
| | | | reductase | | | GO: 6629; lipid metabolism; inferred from electronic annotation |
| | | | | | | GO: 9058; biosynthesis; inferred from electronic annotation |
| | | | | | | GO: 6695; cholesterol biosynthesis; not recorded |
| | | | | | | GO: 8354; germ-cell migration; tracesble author statement” |
| 47 | 202539_s_at | HMGCR | 3-hydroxy-3-methylglutaryl-Coenzyme A | 5q13.3-q14 | Consensussequence | “GO: 8406; gonad development; traceable author statement |
| | | | reductase | | | GO: 6629; lipid metabolism; inferred from electronic annotation |
| | | | | | | GO: 9058; biosynthesis; inferred from electronic annotation |
| | | | | | | GO: 6695; cholesterol biosynthesis; not recorded |
| | | | | | | GO: 8354; germ-cell migration; tracesble author statement” |
| 48 | 200692_s_at | HSPA9B | heat shock 70 kDa protein 9B (mortalin-2) | 5q31.1 | Exemplarsequence | |
| 49 | 200690_at | HSPA9B | heat shock 70 kDa protein 9B (mortalin-2) | 5q31.1 | Consensussequence | |
| 50 | 200691_s_at | HSPA9B | heat shock 70 kDa protein 9B (mortalin-2) | 5q31.1 | Exemplarsequence | |
| 51 | 200692_s_at | HSPA9B | heat shock 70 kDa protein 9B (mortalin-2) | 5q31.1 | Exemplarsequence | |
| 52 | 200690_at | HSPA9B | heat shock 70 kDa protein 9B (mortalin-2) | 5q31.1 | Consensussequence | |
| 53 | 200691_s_at | HSPA9B | heat shock 70 kDa protein 9B (mortalin-2) | 5q31.1 | Exemplarsequence | |
| 54 | 211000_s_at | IL6ST | interleukin 6 signal transducer (gp130, | 5q11 | Exemplarsequence | “GO: 7166; cell surface receptor linked signal |
| | | | oncostatin M receptor) | | | transduction; traceable author statement GO: 6955; immune |
| | | | | | | response; traceable author statement” |
| 55 | 204864_s_at | IL6ST | interleukin 6 signal transducer (gp130, | 5q11 | Exemplarsequence | “GO: 7166; cell surface receptor linked signal |
| | | | oncostatin M receptor) | | | transduction; traceable author statement GO: 6955; immune |
| | | | | | | response; traceable author statement” |
| 56 | 204863_s_at | IL6ST | interleukin 6 signal transducer (gp130, | 5q11 | Consensussequence | “GO: 7166; cell surface receptor linked signal |
| | | | oncostatin M receptor) | | | transduction; traceable author statement GO: 6955; immune |
| | | | | | | response; traceable author statement” |
| 57 | 234474_x_at | IL6ST | interleukin 6 signal transducer (gp130, | 5q11 | Consensussequence | “GO: 7166; cell surface receptor linked signal |
| | | | oncostatin M receptor) | | | transduction; traceable author statement GO: 6955; immune |
| | | | | | | response; traceable author statement” |
| 58 | 234967_at | IL6ST | interleukin 6 signal transducer (gp130, | 5q11 | Consensussequence | “GO: 7166; cell surface receptor linked signal |
| | | | oncostatin M receptor) | | | transduction; traceable author statement GO: 6955; immune |
| | | | | | | response; traceable author statement” |
| 59 | 229817_at | KIAA1281 | KIAA1281 protein | 5q23.2 | Consensussequence | |
| 60 | 232303_at | KIAA1281 | KIAA1281 protein | 5q23.2 | Consensussequence | |
| 61 | 225326_at | KIAA1311 | KIAA1311 protein | 5q32 | Consensussequence | |
| 62 | 243295_at | KIAA1311 | KIAA1311 protein | 5q32 | Consensussequence | |
| 63 | 212193_s_at | LARP | likely ortholog of mouse la related protein | 5q33.2 | Consensussequence | |
| 64 | 210966_x_at | LARP | likely ortholog of mouse la related protein | 5q33.2 | Exemplarsequence | |
| 65 | 239663_x_at | LARP | likely ortholog of mouse la related protein | 5q33.2 | Consensussequence | |
| 66 | 205270_s_at | LCP2 | lymphocyte cytosolic protein 2 (SH2 | 5q33.1-qter | Exemplarsequence | “GO: 7169; transmembrane receptor protein tyrosine kinase |
| | | | domain containing leukocyte protein of | | | signaling pathway; traceable author statement |
| | | | 76 kDa) | | | GO: 7242; intracellular signaling cascade; inferred from |
| | | | | | | electronic annotation GO: 6955; immune response; tracaable |
| | | | | | | author statement” |
| 67 | 205269_at | LCP2 | lymphocyte cytosolic protein 2 (SH2 | 5q33.1-qter | Consensussequence | “GO: 7169; transmembrane receptor protein tyrosine kinase |
| | | | domain containing leukocyte protein of | | | signaling pathway; traceable author statement |
| | | | 76 kDa) | | | GO: 7242; intracellular signaling cascade; inferred from |
| | | | | | | electronic annotation GO: 6955; immune response; tracaable |
| | | | | | | author statement” |
| 68 | 244576_at | LCP2 | lymphocyte cytosolic protein 2 (SH2 | 5q33.1-qter | Consensussequence | “GO: 7169; transmembrane receptor protein tyrosine kinase |
| | | | domain containing leukocyte protein of | | | signaling pathway; traceable author statement |
| | | | 76 kDa) | | | GO: 7242; intracellular signaling cascade; inferred from |
| | | | | | | electronic annotation GO: 6955; immune response; tracaable |
| | | | | | | author statement” |
| 69 | 235208_at | MGC39633 | hypothetical protein MGC39633 | 5q22.2 | Consensussequence | |
| 70 | 213550_s_at | NDUFA2 | NADH dehydrogenase (ubiquinone) 1 | 5q31 | Consensussequence | “GO: 6091; energy pathways; not recorded” |
| | | | alpha subcomptes, 2, 8 kDa | | | |
| 71 | 209224_s_at | NDUFA2 | NADH dehydrogenase (ubiquinone) 1 | 5q31 | Exemplarsequence | “GO: 6091; energy pathways; not recorded” |
| | | | alpha subcomplex, 2, 8 kDa | | | |
| 72 | 209223_at | NDUFA2 | NADH dehydrogenase (ubiquinone) 1 | 5q31 | Consensussequence | “GO: 6091; energy pathways; not recorded” |
| | | | alpha subcomplex, 2, 8 kDa | | | |
| 73 | 201507_at | PFDN1 | prefoldin 1 | 5q31 | Exemplarsequence | “GO: 7049; cell cycle; traceable author statement” |
| 74 | 220746_s_at | RAP80 | receptor associated protein 80 | 5q35.3 | Exemplarsequence | |
| 75 | 207974_s_at | SKP1A | S-phase kinase-associated protein 1A | 5q31 | Exemplarsequence | |
| | | | (p19A) | | | |
| 76 | 200719_at | SKP1A | S-phase kinase-associated protein 1A | 5q31 | Consensussequence | |
| | | | (p19A) | | | |
| 77 | 200718_s_at | SKP1A | S-phase kinase-associated protein 1A | 5q31 | Consensussequence | |
| | | | (p19A) | | | |
| 78 | 200711_s_at | SKP1A | S-phase kinase-associated protein 1A | 5q31 | Exemplarsequence | |
| | | | (p19A) | | | |
| 79 | 205097_at | SLC26A2 | solute carrier family 26 (sulfate | 5q31-q34 | Consensussequence | “GO: 8272; sulfate transport; traceable author statement |
| | | | transporter), member 2 | | | GO: 6810; transport; inferred from electronic annotation” |
| 80 | 202114_at | SNX2 | sorting nexin 2 | 5q23 | Exemplarsequence | “GO: 6897; endocytosis; traceable author statement |
| | | | | | | GO: 6886; intracellular protein transport; inferred from electronic |
| | | | | | | annotation GO: 7242; intracellular signaling cascade; inferred |
| | | | | | | from electronic annotation” |
| 81 | 202113_s_at | SNX2 | sorting nexin 2 | 5q23 | Exemplarsequence | “GO: 6897; endocytosis; traceable author statement |
| | | | | | | GO: 6886; intracellular protein transport; inferred from electronic |
| | | | | | | annotation GO: 7242; intracellular signaling cascade; inferred |
| | | | | | | from electronic annotation” |
| 82 | 235737_at | TSLP | thymic stromal lymphopoietin | 5q21.3 | Consensussequence | |

| affy id | Go_Molecular_Function | Transcript ID | Sequence Derived From |
|---|---|---|---|
| 1 | | “GO: 3754; chaperone activity; inferred from electronic annotation GO: 46872; metal ion binding; inferred from | Hs.279910.0 |
| | | electronic annotation GO: 5507; copper ion binding; traceable author statement GO: 5505; HMA; heavy metal | |
| | | binding activity; 1.2e−11; extended: Unknown” | |
| 2 | | | Hs.142827.0 |
| 3 | | | Hs.142827.0 |
| 4 | | | Hs.120969.0 |
| 5 | | | 4861120 |
| 6 | | | 4861120 |
| 7 | | | Hs.10235.0 |
| 8 | | “GO: 5096; GTPase activator activity; inferred from electronic annotation” | Hs.82035.0 |
| 9 | “GO: 5634; nucleus; non-traceable author statement” | “GO: 3700; transcription factor activity; non-traceable author statement” | Hs.26703.0 |
| 10 | “GO: 5634; nucleus; non-traceable author | “GO: 3700; transcription factor activity; non-traceable author statement” | Hs.26703.0 |
| | statement” | | |
| 11 | “GO: 5634; nucleus; non-traceable author statement” | “GO: 3700; transcription factor activity; non-traceable author statement” | Hs.26703.0 |
| 12 | “GO: 5739; mitochondrion; inferred from | “GO: 9482; ba3-type cytochrome c oxidase; inferred from electronic annotation GO: 9481; aa3-type | Hs.278494.0 |
| | electronic annotation” | cytochrome c oxidase; inferred from electronic annotation GO: 9483; caa3-type cytochrome c | |
| | | oxidase; inferred from electronic annotation GO: 4129; cytochrome c oxidase activity; inferred from electronic | |
| | | annotation GO: 16491; oxidoreductase activity; inferred from electronic annotation GO: 9485; cbb3-type | |
| | | cytochrome c oxidase; inferred from electronic annotation” | |
| 13 | “GO: 5739; mitochondrion; inferred from | “GO: 9482; ba3-type cytochrome c oxidase; inferred from electronic annotation GO: 9481; aa3-type | Hs.3462.1 |
| | electronic annotation” | cytochrome c oxidase; inferred from electronic annotation GO: 9483; caa3-type cytochrome c | |
| | | oxidase; inferred from electronic annotation GO: 4129; cytochrome c oxidase activity; inferred from electronic | |
| | | annotation GO: 16491; oxidoreductase activity; inferred from electronic annotation GO: 9485; cbb3-type | |
| | | cytochrome c oxidase; inferred from electronic annotation” | |
| 14 | “GO: 5739; mitochondrion; inferred from | “GO: 9482; ba3-type cytochrome c oxidase; inferred from electronic annotation GO: 9481; aa3-type | Hs.3462.0 |
| | electronic annotation” | cytochrome c oxidase; inferred from electronic annotation GO: 9483; caa3-type cytochrome c | |
| | | oxidase; inferred from electronic annotation GO: 4129; cytochrome c oxidase activity; inferred from electronic | |
| | | annotation GO: 16491; oxidoreductase activity; inferred from electronic annotation GO: 9485; cbb3-type | |
| | | cytochrome c oxidase; inferred from electronic annotation” | |
| 15 | | “GO: 4681; casein kinase I activity; experimental evidence GO: 4672; pkinase; protein kinase activity; 1.3e−27; | Hs.283738.1 |
| | | extended: inferred from electronic annotation” | |
| 16 | | “GO: 4681; casein kinase I activity; experimental evidence GO: 4672; pkinase; protein kinase activity; 1.3e−27; | Hs.84264.3 |
| | | extended: inferred from electronic annotation” | |
| 17 | | “GO: 4681; casein kinase I activity; experimental evidence GO: 4672; pkinase; protein kinase activity; 1.3e−27; | Hs.144477.0 |
| | | extended: inferred from electronic annotation” | |
| 18 | | “GO: 4681; casein kinase I activity; experimental evidence GO: 4672; pkinase; protein kinase activity; 1.3e−27; | Hs.144477.0 |
| | | extended: inferred from electronic annotation” | |
| 19 | | “GO: 4681; casein kinase I activity; experimental evidence GO: 4672; pkinase; protein kinase activity; 1.3e−27; | Hs.144477.0 |
| | | extended: inferred from electronic annotation” | |
| 20 | | “GO: 4681; casein kinase I activity; experimental evidence GO: 4672; pkinase; protein kinase activity; 1.3e−27; | Hs.283738.0 |
| | | extended: inferred from electronic annotation” | |
| 21 | | “GO: 4681; casein kinase I activity; experimental evidence GO: 4672; pkinase; protein kinase activity; 1.3e−27; | Hs.104216.0 |
| | | extended: inferred from electronic annotation” | |
| 22 | | | Hs.59666.0 |
| 23 | “GO: 5813; centrosome; predicted/computed | | Hs.180952.0 |
| | GO: 5869; dynactin complex; not | | |
| | recorded | | |
| | GO: 5634; nucleus; predicted/computed | | |
| | GO: 5737; cytoplasm; predicted/computed” | | |
| 24 | “GO: 5813; centrosome; predicted/computed | | Hs.180952.0 |
| | GO: 5869; dynactin complex; not | | |
| | recorded | | |
| | GO: 5634; nucleus; predicted/computed | | |
| | GO: 5737; cytoplasm; predicted/computed” | | |
| 25 | “GO: 5813; centrosome; predicted/computed | | Hs.180952.2 |
| | GO: 5869; dynactin complex; not | | |
| | recorded | | |
| | GO: 5634; nucleus; predicted/computed | | |
| | GO: 5737; cytoplasm; predicted/computed” | | |
| 26 | “GO: 5813; centrosome; predicted/computed | | Hs.180952.0 |
| | GO: 5869; dynactin complex; not | | |
| | recorded | | |
| | GO: 5634; nucleus; predicted/computed | | |
| | GO: 5737; cytoplasm; predicted/computed” | | |
| 27 | “GO: 5813; centrosome; predicted/computed | | Hs.180952.0 |
| | GO: 5869; dynactin complex; not | | |
| | recorded | | |
| | GO: 5634; nucleus; predicted/computed | | |
| | GO: 5737; cytoplasm; predicted/computed” | | |
| 28 | “GO: 5813; centrosome; predicted/computed | | Hs.180952.2 |
| | GO: 5869; dynactin complex; not | | |
| | recorded | | |
| | GO: 5634; nucleus; predicted/computed | | |
| | GO: 5737; cytoplasm; predicted/computed” | | |
| 29 | | | Hs.272314.0 |
| 30 | | | Hs.77798.1 |
| 31 | | | 2 |
| 32 | | | Hs.326464.1 |
| 33 | | | Hs.326464.0 |
| 34 | | | Hs.286261.0 |
| 35 | | | Hs.107622.0 |
| 36 | | | Hs.107260.1 |
| 37 | | | Hs.107260.0 |
| 38 | | | Hs.9286.0 |
| 39 | “GO: 5764; lysosome; non-traceable author | “GO: 30290; sphingolipid activator protein activity; non-traceable author statement” | 4898374 |
| | statement” | | |
| 40 | “GO: 5764; lysosome; non-traceable author | “GO: 30290; sphingolipid activator protein activity; non-traceable author statement” | 4819721 |
| | statement” | | |
| 41 | “GO: 5764; lysosome; non-traceable author | “GO: 30290; sphingolipid activator protein activity; non-traceable author statement” | Hs.289082.1 |
| | statement” | | |
| 42 | “GO: 5764; lysosome; non-traceable author | “GO: 30290; sphingolipid activator protein activity; non-traceable author statement” | Hs.289082.1 |
| | statement” | | |
| 43 | “GO: 5764; lysosome; non-traceable author | “GO: 30290; sphingolipid activator protein activity; non-traceable author statement” | Hs.278242.3 |
| | statement” | | |
| 44 | “GO: 5764; lysosome; non-traceable author | “GO: 30290; sphingolipid activator protein activity; non-traceable author statement” | Hs.289082.0 |
| | statement” | | |
| 45 | “GO: 5634; nucleus; traceable author | “GO: 4407; histone deacetylase activity; traceable author statement GO: 8189; apoptosis inhibitor | Hs.279789.12 |
| | statement” | activity; traceable author statement GO: 16787; hydrolase activity; inferred from electronic annotation” | |
| 46 | “GO: 5624; membrane fraction; not | “GO: 4420; hydroxymethylglutaryl-CoA reductase (NADPH) activity; inferred from electronic annotation | Hs.11899.0 |
| | recorded GO: 5777; peroxisome; inferred | GO: 16491; oxidoreductase activity; inferred from electronic annotation” | |
| | from electronic annotation | | |
| | GO: 5789; endoplasmic reticulum | | |
| | membrane; traceable author statement | | |
| | GO: 16021; integral to membrane; inferred | | |
| | from electronic annotation” | | |
| 47 | “GO: 5624; membrane fraction; not | “GO: 4420; hydroxymethylglutaryl-CoA reductase (NADPH) activity; inferred from electronic annotation | Hs.11899.0 |
| | recorded GO: 5777; peroxisome; inferred | GO: 16491; oxidoreductase activity; inferred from electronic annotation” | |
| | from electronic annotation | | |
| | GO: 5789; endoplasmic reticulum | | |
| | membrane; traceable author statement | | |
| | GO: 16021; integral to membrane; inferred | | |
| | from electronic annotation” | | |
| 48 | “GO: 5739; mitochondrion; traceable author | “GO: 5524; ATP binding; inferred from electronic annotation” | Hs.3069.0 |
| | statement” | | |
| 49 | “GO: 5739; mitochondrion; traceable author | “GO: 5524; ATP binding; inferred from electronic annotation” | Hs.3069.0 |
| | statement” | | |
| 50 | “GO: 5739; mitochondrion; traceable author | “GO: 5524; ATP binding; inferred from electronic annotation” | Hs.3069.0 |
| | statement” | | |
| 51 | “GO: 5739; mitochondrion; traceable author | “GO: 5524; ATP binding; inferred from electronic annotation” | Hs.3069.0 |
| | statement” | | |
| 52 | “GO: 5739; mitochondrion; traceable author | “GO: 5524; ATP binding; inferred from electronic annotation” | Hs.3069.0 |
| | statement” | | |
| 53 | “GO: 5739; mitochondrion; traceable author | “GO: 5524; ATP binding; inferred from electronic annotation” | Hs.3069.0 |
| | statement” | | |
| 54 | “GO: 5887; integral to plasma | “GO: 4898; gp130; traceable author statement GO: 4924; oncostatin-M receptor activity; traceable author | Hs.82065.1 |
| | membrane; traceable author statement” | statement GO: 4872; receptor activity; traceable author statement” | |
| 55 | “GO: 5887; integral to plasma | “GO: 4898; gp130; traceable author statement GO: 4924; oncostatin-M receptor activity; traceable author | Hs.82065.0 |
| | membrane; traceable author statement” | statement GO: 4872; receptor activity; traceable author statement” | |
| 56 | “GO: 5887; integral to plasma | “GO: 4898; gp130; traceable author statement GO: 4924; oncostatin-M receptor activity; traceable author | Hs.82065.0 |
| | membrane; traceable author statement” | statement GO: 4872; receptor activity; traceable author statement” | |
| 57 | “GO: 5887; integral to plasma | “GO: 4898; gp130; traceable author statement GO: 4924; oncostatin-M receptor activity; traceable author | Hs.283974.0 |
| | membrane; traceable author statement” | statement GO: 4872; receptor activity; traceable author statement” | |
| 58 | “GO: 5887; integral to plasma | “GO: 4898; gp130; traceable author statement GO: 4924; oncostatin-M receptor activity; traceable author | Hs.283974.0 |
| | membrane; traceable author statement” | statement GO: 4872; receptor activity; traceable author statement” | |
| 59 | | | Hs.93738.0 |
| 60 | | | Hs.42796.0 |
| 61 | | “GO: 3676; nucleic acid binding; inferred from electronic annotation” | Hs.61441.0 |
| 62 | | “GO: 3676; nucleic acid binding; inferred from electronic annotation” | Hs.183412.0 |
| 63 | | | Hs.6214.1 |
| 64 | | | Hs.6214.0 |
| 65 | | | Hs.207988.0 |
| 66 | | “GO: 5515; protein binding; traceable author statement” | Hs.2488.0 |
| 67 | | “GO: 5515; protein binding; traceable author statement” | Hs.2488.0 |
| 68 | | “GO: 5515; protein binding; traceable author statement” | Hs.260872.0 |
| 69 | | | Hs.47534.0 |
| 70 | “GO: 5739; mitochondrion; inferred from | “GO: 3954; NADH dehydrogenase activity; inferred from electronic annotation GO: 16491; oxidoreductase | Hs.163867.1 |
| | electronic annotation | activity; inferred from electronic annotation GO: 8137; NADH dehydrogenase (ubiquinone) activity; traceable | |
| | GO: 5624; membrane fraction; not | author statement” | |
| | recorded” | | |
| 71 | “GO: 5739; mitochondrion; inferred from | “GO: 3954; NADH dehydrogenase activity; inferred from electronic annotation GO: 16491; oxidoreductase | Hs.163867.0 |
| | electronic annotation | activity; inferred from electronic annotation GO: 8137; NADH dehydrogenase (ubiquinone) activity; traceable | |
| | GO: 5624; membrane fraction; not | author statement” | |
| | recorded” | | |
| 72 | “GO: 5739; mitochondrion; inferred from | “GO: 3954; NADH dehydrogenase activity; inferred from electronic annotation GO: 16491; oxidoreductase | Hs.163867.0 |
| | electronic annotation | activity; inferred from electronic annotation GO: 8137; NADH dehydrogenase (ubiquinone) activity; traceable | |
| | GO: 5624; membrane fraction; not | author statement” | |
| | recorded” | | |
| 73 | | “GO: 3754; chaperone activity; inferred from electronic annotation GO: 3700; transcription factor | Hs.132881.0 |
| | | activity; traceable author statement” | |
| 74 | | | Hs.7889.0 |
| 75 | | | Hs.227950.0 |
| 76 | | | Hs.171626.0 |
| 77 | | | Hs.171626.0 |
| 78 | | | Hs.171626.0 |
| 79 | “GO: 5887; integral to plasma | “GO: 8271; sulfate porter activity; inferred from electronic annotation” | Hs.29981.0 |
| | membrane; traceable author statement | | |
| | GO: 5624; membrane fraction; traceable | | |
| | author statement” | | |
| 80 | | “GO: 8565; protein transporter activity; inferred from electronic annotation” | Hs.11183.0 |
| 81 | | “GO: 8565; protein transporter activity; inferred from electronic annotation” | Hs.11183.0 |
| 82 | | | Hs.128051.0 |

| affy id | Sequence ID | Sequence Source | Unigene_Accession | Cluster_Type | LocusLink | Full_Length_Reference_Seq | |
|---|---|---|---|---|---|---|---|
| 1 | NM_004045.1 | g4757803 | RefSeq | Hs.279910 | fulllength | 475 | NM_004045; antioxidant protein 1 |
| 2 | NM_004772.1 | g4758865 | RefSeq | Hs.413760 | fulllength | 9315 | NM_004772; neuronal protein 3.1 |
| 3 | U36189.1 | g1244509 | GenBank | Hs.413760 | fulllength | 9315 | NM_004772; neuronal protein 3.1 |
| 4 | AU144860 | Hs.120969.0 | GenBank | Hs.413760 | fulllength | 9315 | NM_004772; neuronal protein 3.1 |
| 5 | H93077 | 4861120 | GenBank | Hs.10235 | fulllength | 10826 | NM_016348; chromosome 5 open reading frame 4 |
| | | | | | | | NM_032385; chromosome 5 open reading frame 4 |
| 6 | H93077 | 4861120 | GenBank | Hs.10235 | fulllength | 10826 | NM_016348; chromosome 5 open reading frame 4 |
| | | | | | | | NM_032385; chromosome 5 open reading frame 4 |
| 7 | NM_016348.1 | g7705942 | RefSeq | Hs.10235 | fulllength | 10826 | NM_016348; chromosome 5 open reading frame 4 |
| | | | | | | | NM_032385; chromosome 5 open reading frame 4 |
| 8 | NM_016603.1 | g7706136 | RefSeq | Hs.82035 | fulllength | 51306 | NM_016603; chromosome 5 open reading frame 5 |
| 9 | AF180476.1 | g6856208 | GenBank | Hs.26703 | fulllength | 9337 | NM_004779; CCR4-NOT transcription complex, subunit 8 |
| 10 | NM_004779.1 | g4758945 | RefSeq | Hs.26703 | fulllength | 9337 | NM_004779; CCR4-NOT transcription complex, subunit 8 |
| 11 | AI769416 | Hs.26703.0_RC | GenBank | Hs.26703 | fulllength | 9337 | NM_004779; CCR4-NOT transcription complex, subunit 8 |
| 12 | AF042165 | Hs.278494.0 | GenBank | Hs.430075 | fulllength | 1350 | NM_001867; cytochrome c oxidase subunit VIIc precursor |
| 13 | AA382702 | Hs.3462.1 | GenBank | Hs.430075 | fulllength | 1350 | NM_001867; cytochrome c oxidase subunit VIIc precursor |
| 14 | NM_001867.1 | g4502992 | RefSeq | Hs.430075 | fulllength | 1350 | NM_001867; cytochrome c oxidase subunit VIIc precursor |
| 15 | AW592437 | Hs.283738.1.A1 | GenBank | Hs.283738 | fulllength | 1452 | NM_001892; casein kinase 1, alpha 1 NM_018548; |
| 16 | BF341845 | Hs.84264.3.A1 | GenBank | Hs.283738 | fulllength | 1452 | NM_001892; casein kinase 1, alpha 1 NM_018548; |
| 17 | AF119911.1 | g7770258_RC | GenBank | Hs.283738 | fulllength | 1452 | NM_001892; casein kinase 1, alpha 1 NM_018548; |
| 18 | BG534245 | Hs.144477.0.S2 | GenBank | Hs.283738 | fulllength | 1452 | NM_001892; casein kinase 1, alpha 1 NM_018548; |
| 19 | BG534245 | Hs.144477.0.S2 | GenBank | Hs.283738 | fulllength | 1452 | NM_001892; casein kinase 1, alpha 1 NM_018548; |
| 20 | NM_001892.1 | g4503088 | RefSeq | Hs.283738 | fulllength | 1452 | NM_001892; casein kinase 1, alpha 1 NM_018548; |
| 21 | AA010757 | Hs.104216.0.A1 | GenBank | Hs.283738 | fulllength | 1452 | NM_001892; casein kinase 1, alpha 1 NM_018548; |
| 22 | NM_022001.1 | g11545925 | RefSeq | Hs.59666 | fulllength | 9597 | NM_022001; SMAD in the antisense orientation |
| 23 | BE218028 | Hs.180952.0_RC | GenBank | Hs.328865 | fulllength | 51164 | NM_016221; dynactin 4 (p62) |
| 24 | NM_016221.1 | g7705892 | RefSeq | Hs.328865 | fulllength | 51164 | NM_016221; dynactin 4 (P62) |
| 25 | AI985890 | Hs.180952.2_RC | GenBank | Hs.328865 | fulllength | 51164 | NM_016221; dynactin 4 (p62) |
| 26 | BE218028 | Hs.180952.0_RC | GenBank | Hs.328865 | fulllength | 51164 | NM_016221; dynactin 4 (p62) |
| 27 | NM_016221.1 | g7705892 | RefSeq | Hs.328865 | fulllength | 51164 | NM_016221; dynactin 4 (p62) |
| 28 | AI985890 | Hs.180952.2_RC | GenBank | Hs.328865 | fulllength | 51164 | NM_016221; dynactin 4 (p62) |
| 29 | AL133602.1 | Hs.272314.0 | GenBank | Hs.272314 | | 64839 | |
| 30 | BC002482.1 | g12803328 | GenBank | Hs.286261 | fulllength | 54853 | NM_017706; hypothetical protein FLJ20195 |
| 31 | AI949010 | 4919314_rc | GenBank | Hs.286261 | fulllength | 54853 | NM_017706; hypothetical protein FLJ20195 |
| 32 | BE409994 | Hs.326464.1 | GenBank | Hs.286261 | fulllength | 54853 | NM_017706; hypothetical protein FLJ20195 |
| 33 | AW090043 | Hs.326464.0.A1 | GenBank | Hs.286261 | fulllength | 54853 | NM_017706; hypothetical protein FLJ20195 |
| 34 | NM_017706.1 | g8923184 | RefSeq | Hs.286261 | fulllength | 54853 | NM_017706; hypothetical protein FLJ20195 |
| 35 | AI391443 | Hs.107622.0.A1 | GenBank | Hs.107622 | fulllength | 153443 | NM_152546; hypothetical protein FLJ25286 |
| 36 | BE906572 | Hs.107260.1 | GenBank | Hs.107260 | fulllength | 55568 | NM_017540; UDP-N-acetyl-alpha-D- |
| | | | | | | | galactosamine:polypeptide N- |
| | | | | | | | acetylgalactosaminyltransferase 10 (GalNAc-T10) |
| 37 | NM_017540.1 | g9055207 | RefSeq | Hs.107260 | fulllength | 55568 | NM_017540; UDP-N-acetyl-alpha-D |
| | | | | | | | galactosamine:polypeptide N- |
| | | | | | | | acetylgalactosaminyltransferase 10 (GalNAc-T10) |
| 38 | AK024931.1 | Hs.9286.0.S1 | GenBank | Hs.107260 | fulllength | 55568 | NM_017540; UDP-N-acetyl-alpha-D- |
| | | | | | | | galactosamine:polypeptide N- |
| | | | | | | | acetylgalactosaminyltransferase 10 (GalNAc-T10) |
| 39 | X62078 | 4898374 | GenBank | Hs.289082 | fulllength | 2760 | NM_000405; GM2 ganglioside activator protein precursor |
| 40 | X61094 | 4819721 | GenBank | Hs.289082 | fulllength | 2760 | NM_000405; GM2 ganglioside activator protein precursor |
| 41 | X61094.1 | Hs.289082.1 | GenBank | Hs.289082 | fulllength | 2760 | NM_000405; GM2 ganglioside activator protein precursor |
| 42 | X61094.1 | Hs.289082.1 | GenBank | Hs.289082 | fulllength | 2760 | NM_000405; GM2 ganglioside activator protein precursor |
| 43 | AL513583 | Hs.278242.3 | GenBank | Hs.289082 | fulllength | 2760 | NM_000405; GM2 ganglioside activator protein precursor |
| 44 | M76477.1 | g183356 | GenBank | Hs.289082 | fulllength | 2760 | NM_000405; GM2 ganglioside activator protein precursor |
| 45 | AF059650 | Hs.279789.12.S1 | GenBank | Hs.446552 | fulllength | 8841 | NM_003883; histone deacetylase 3 |
| 46 | NM_000859.1 | g4557642 | RefSeq | Hs.11899 | fulllength | 3156 | NM_000859; 3-hydroxy-3-methylglutaryl-Coenzyme A |
| | | | | | | | reductase |
| 47 | AL518627 | Hs.11899.0 | GenBank | Hs.11899 | fulllength | 3156 | NM_000859; 3-hydroxy-3-methylglutaryl-Coenzyme A |
| | | | | | | | reductase |
| 48 | NM_004134.1 | g4758569 | RefSeq | Hs.3069 | fulllength | 3313 | NM_004134; heat shock 70 kDa protein 9B precursor |
| 49 | AA927701 | Hs.3069.0_RC | GenBank | Hs.3069 | fulllength | 3313 | NM_004134; heat shock 70 kDa protein 9B precursor |
| 50 | BC000478.1 | g12653414 | GenBank | Hs.3069 | fulllength | 3313 | NM_004134; heat shock 70 kDa protein 9B precursor |
| 51 | NM_004134.1 | g4758569 | RefSeq | Hs.3069 | fulllength | 3313 | NM_004134; heat shock 70 kDa protein 9B precursor |
| 52 | AA927701 | Hs.3069.0_RC | GenBank | Hs.3069 | fulllength | 3313 | NM_004134; heat shock 70 kDa protein 9B precursor |
| 53 | BC000478.1 | g12653414 | GenBank | Hs.3069 | fulllength | 3313 | NM_004134; heat shock 70 kDa protein 9B precursor |
| 54 | AB015706.1 | g4972293 | GenBank | Hs.82065 | fulllength | 3572 | NM_002184; interleukin 6 signal transducer isoform 1 |
| | | | | | | | precursor NM_175767; interleukin 6 signal transducer |
| | | | | | | | isoform 2 precursor |
| 55 | NM_002184.1 | g4504674 | RefSeq | Hs.82065 | fulllength | 3572 | NM_002184; interleukin 6 signal transducer isoform 1 |
| | | | | | | | precursor NM_175767; interleukin 6 signal transducer |
| | | | | | | | isoform 2 precursor |
| 56 | BE856546 | Hs.82065.0_RC | GenBank | Hs.82065 | fulllength | 3572 | NM_002184; interleukin 6 signal transducer isoform 1 |
| | | | | | | | precursor NM_175767; interleukin 6 signal transducer |
| | | | | | | | isoform 2 precursor |
| 57 | U58146.1 | Hs.283974.0.A1 | GenBank | Hs.82065 | fulllength | 3572 | NM_002184; interleukin 6 signal transducer isoform 1 |
| | | | | | | | precursor NM_175767; interleukin 6 signal transducer |
| | | | | | | | isoform 2 precursor |
| 58 | U58146.1 | Hs.283974.0.S1 | GenBank | Hs.82065 | fulllength | 3572 | NM_002184; interleukin 6 signal transducer isoform 1 |
| | | | | | | | precursor NM_175767; interleukin 6 signal transducer |
| | | | | | | | isoform 2 precursor |
| 59 | AI452715 | Hs.93738.0.S1 | GenBank | Hs.42796 | | 57507 | |
| 60 | AB033107.1 | Hs.42796.0.S1 | GenBank | Hs.42796 | | 57507 | |
| 61 | AB037732.1 | Hs.61441.0.S1 | GenBank | Hs.61441 | | 54439 | |
| 62 | N75450 | Hs.183412.0.A1 | GenBank | Hs.61441 | | 54439 | |
| 63 | BE881529 | Hs.6214.1.A1 | GenBank | Hs.6214 | fulllength | 23367 | NM_015315; KIAA0731 protein |
| 64 | BC001460.1 | g12655204 | GenBank | Hs.6214 | fulllength | 23367 | NM_015315; KIAA0731 protein |
| 65 | AI768454 | Hs.207988.0.A1 | GenBank | Hs.6214 | fulllength | 23367 | NM_015315; KIAA0731 protein |
| 66 | NM_005565.2 | g7382491 | RefSeq | Hs.2488 | fulllength | 3937 | NM_005565; lymphocyte cytosolic protein 2 |
| 67 | AI123251 | Hs.2488.0.S2 | GenBank | Hs.2488 | fulllength | 3937 | NM_005565; lymphocyte cytosolic protein 2 |
| 68 | AA992040 | Hs.260872.0_RC | GenBank | Hs.2488 | fulllength | 3937 | NM_005565; lymphocyte cytosolic protein 2 |
| 69 | AW157712 | Hs.47534.0.A1 | GenBank | Hs.47534 | fulllength | 153733 | NM_152549; hypothetical protein MGC39633 |
| 70 | AA993683 | Hs.163867.1_RC | GenBank | Hs.163867 | fulllength | 4695 | NM_002488; NADH dehydrogenase (ubiquinone) 1 alpha |
| | | | | | | | subcomplex, 2, 8 kDa |
| 71 | BC003674.1 | g13277539 | GenBank | Hs.163867 | fulllength | 4695 | NM_002488; NADH dehydrogenase (ubiquinone) 1 alpha |
| | | | | | | | subcomplex, 2, 8 kDa |
| 72 | BF434335 | Hs.163867.0.S1 | GenBank | Hs.163867 | fulllength | 4695 | NM_002488; NADH dehydrogenase (ubiquinone) 1 alpha |
| | | | | | | | subcomplex, 2, 8 kDa |
| 73 | NM_002622.2 | g12408673 | RefSeq | Hs.132881 | fulllength | 5201 | NM_002622; prefoldin 1 |
| 74 | NM_016290.1 | g7706482 | RefSeq | Hs.7889 | fulllength | 51720 | NM_016290; retinoid x receptor interacting protein |
| 75 | NM_006930.1 | g5902087 | RefSeq | Hs.171626 | fulllength | 6500 | NM_006930; S-phase kinase-associated protein 1A isoform |
| | | | | | | | a NM_170679; S-phase kinase-associated protein 1A |
| | | | | | | | isoform b |
| 76 | NM_003197.2 | Hs.171626.0 | GenBank | Hs.171626 | fulllength | 6500 | NM_006930; S-phase kinase-associated protein 1A isoform |
| | | | | | | | a NM_170679; S-phase kinase-associated protein 1A |
| | | | | | | | isoform b |
| 77 | NM_003197.2 | Hs.171626.0 | GenBank | Hs.171626 | fulllength | 6500 | NM_006930; S-phase kinase-associated protein 1A isoform |
| | | | | | | | a NM_170679; S-phase kinase-associated protein 1A |
| | | | | | | | isoform b |
| 78 | NM_003197.2 | g6006030 | RefSeq | Hs.171626 | fulllength | 6500 | NM_006930; S-phase kinase-associated protein 1A isoform |
| | | | | | | | a NM_170679; S-phase kinase-associated protein 1A |
| | | | | | | | isoform b |
| 79 | AI025519 | Hs.29981.0 | GenBank | Hs.29981 | fulllength | 1836 | NM_000112; sulfate anion transporter 1 |
| 80 | NM_003100.1 | g4507140 | RefSeq | Hs.11183 | fulllength | 6643 | NM_003100; sorting nexin 2 |
| 81 | AF043453.1 | g2827433 | GenBank | Hs.11183 | fulllength | 6643 | NM_003100; sorting nexin 2 |
| 82 | AW118681 | Hs.128051.0.A1 | GenBank | Hs.128051 | fulllength | 85480 | NM_033035; thymic stromal lymphopoietin isoform 1 |
| | | | | | | | NM_138551; thymic stromal lymphopoietin isoform 2 |

TABLE 42
Top 50 differentially expressed genes in subjects with or without 5q-deletions

| # | affy id | HUGO name | fc | p | q | stn | t | Title |
|---|---|---|---|---|---|---|---|---|
| 1 | 223157_at | MGC3232 | −176,840,673,711,012 | 1.56E−30 | 2.27E−26 | −0.878761187518287 | −16,290,783,098,029 | hypothetical protein MGC3232 |
| 2 | 227056_at | | −210,270,102,717,117 | 4.23E−23 | 3.07E−20 | −0.835901800343154 | −149,859,562,558,106 | ESTs |
| 3 | 222983_s_at | PAIP2 | −152,774,472,098,093 | 2.60E−22 | 1.26E−18 | −0.824763060632146 | −14,774,791,378,756 | PABP-interacting protein 2 |
| 4 | 222229_x_at | | −138,555,045,872,062 | 1.32E−19 | 3.84E−16 | −0.857704454194318 | −146,682,149,163,202 | |
| 5 | 200093_s_at- | HINT1 | −167,652,977,392,515 | 1.06E−19 | 3.84E−16 | −0.775962891073544 | −139,632,025,220,666 | histidine triad nucleotide binding protein 1 |
| | HG-U133B | | | | | | | |
| 6 | 208826_x_at | HINT1 | −149,623,902,638,172 | 3.44E−17 | 7.13E−14 | −0.72041319782511 | −131,083,164,795,885 | histidine triad nucleotide binding |
| | | | | | | | | protein 1 |
| 7 | 200093_s_at- | HINT1 | −155,871,940,613,387 | 1.40E−16 | 2.54E−13 | −0.717122701625737 | −129,921,366,659,411 | histidine triad nucleotide binding |
| | HG-U133A | | | | | | | protein 1 |
| 8 | 218436_at | SIL1 | −227,341,823,543,551 | 1.47E−17 | 3.56E−14 | −0.701444914476295 | −129,888,069,023,019 | endoplasmic reticulum chaperone |
| | | | | | | | | SIL1, homolog of yeast |
| 9 | 218645_at | ZNF277 | −176,295,972,337,266 | 5.89E−16 | 9.50E−13 | −0.710642779487633 | −128,442,564,651,243 | zinc finger protein (C2H2 type) 277 |
| 10 | 201922_at | YR-29 | −142,354,413,066,903 | 1.25E−15 | 1.51E−12 | −0.734143241558063 | −128,308,922,616,193 | hypothetical protein YR-29 |
| 11 | 213846_at | COX7C | −168,767,608,406,954 | 6.22E−16 | 8.21E−12 | −0.719989960058898 | −127,660,986,978,765 | cytochrome c oxsidase subunit VIIc |
| 12 | 236892_s_at | | −559,327,252,933,867 | 1.98E−14 | 2.21E−11 | −0.715366073440501 | −124,862,044,859,403 | Homo sapiens, clone MGC: 10077 |
| | | | | | | | | IMAGE: 3896690, mRNA, complete |
| | | | | | | | | cds |
| 13 | 201574_at | ETF1 | −161,004,106,965,863 | 2.94E−15 | 4.26E−12 | −0.676198023006729 | −124,433,769,785,292 | eukaryotic translation termination |
| | | | | | | | | factor 1 |
| 14 | 223276_at | NID67 | −216,267,559,712,988 | 3.04E−14 | 3.15E−11 | −0.656161193924451 | −121,237,932,293,719 | putative small membrane protein |
| | | | | | | | | NID67 |
| 15 | 239791_at | | −547,776,290,365,847 | 2.96E−13 | 2.86E−10 | −0.672006730873876 | −120,352,411,255,962 | Homo sapiens, clone MGC: 10077 |
| | | | | | | | | IMAGE: 3896690, mRNA, complete |
| | | | | | | | | cds |
| 16 | 205366_s_at | HOXB6 | −44,911,335,922,303 | 3.49E−13 | 3.16E−10 | −0.655406699380282 | −119,281,404,087,584 | homeo box B6 |
| 17 | 201978_s_at | KIAA0141 | −191,317,509,860,742 | 4.12E−13 | 3.51E−10 | −0.639216462493315 | −11,812,517,148,329 | KIAA0141 gene product |
| 18 | 205382_s_at | DF | −293,432,627,776,163 | 1.11E−12 | 8.94E−10 | −0.629029607893431 | −116,633,749,020,449 | D component of complement (adipsin) |
| 19 | 201345_s_at | UBE2D2 | −148,262,114,288,443 | 1.26E−11 | 9.16E−09 | −0.642126647798994 | −116,009,609,837,515 | ubiquitin-conjugating enzyme E2D 2 |
| | | | | | | | | (UBC4/5 homolog, yeast) |
| 20 | 202413_s_at | USP1 | 15,550,121,473,553 | 3.10E−10 | 2.05E−07 | 0.667099477655205 | 115,393,157,069,109 | ubiquitin specific protease 1 |
| 21 | 217751_at | LOC51064 | −191,941,596,685,024 | 7.51E−13 | 5.74E−09 | −0.618116585891762 | −11,445,907,258,588 | glutathione S-transferase subunit 13 |
| | | | | | | | | homolog |
| 22 | 207721_x_at | HINT1 | −161,968,228,706,642 | 2.68E−11 | 1.85E−08 | −0.628345318200291 | −11,438,552,379,395 | histidine triad nucleotide binding |
| | | | | | | | | protein 1 |
| 23 | 241827_at | | 158,826,758,599,576 | 3.51E−09 | 1.88E−07 | 0.68393425971076 | 114,342,004,860,552 | clone PEBLM2007832, moderately |
| | | | | | | | | similar to ZINC FINGER PROTEIN 84. |
| 24 | 200608_s_at | RAD21 | 143,095,680,451,226 | 1.15E−08 | 5.56E−06 | 0.688201897055249 | 113,449,542,535,785 | RAD21 homolog (S. pombe) |
| 25 | 201977_s_at | KIAA0141 | −153,332,193,867,581 | 5.32E−10 | 3.22E−07 | −0.610461380259132 | −110,828,336,270,582 | KIAA0141 gene product |
| 26 | 203538_at | CAMLG | −151,834,530,468,475 | 4.42E−10 | 2.79E−07 | −0.606086007348918 | −110,631,682,617,063 | calcium modulating ligand |
| 27 | 208646_at | RPS14 | −173,979,392,536,201 | 2.34E−09 | 1.30E−06 | −0.6127387743923 | −10,987,412,116,646 | ribosomal protein S14 |
| 28 | 222902_s_at | FLJ21144 | 161,896,681,826,594 | 6.42E−07 | 2.33E−04 | 0.69463366257089 | 109,496,234,449,283 | hypothetical protein FLJ21144 |
| 29 | 226835_s_at | | −165,273,462,862,669 | 1.32E−09 | 7.66E−09 | −0.595761971718935 | −109,038,850,423,911 | Homo sapiens, clone |
| | | | | | | | | IMAGE: 5285034, mRNA |
| 30 | 200066_at-HG- | IK | −143,128,055,082,803 | 1.30E−08 | 6.10E−07 | −0.615394465733112 | −108,685,620,572,657 | IK cytokine, down-regulator of HLA II |
| | U133B | | | | | | | |
| 31 | 221476_s_at | RPL15 | −127,049,845,993,301 | 5.02E−08 | 2.14E−05 | −0.63269486669309 | −108,678,589,099,167 | ribosomal protein L15 |
| 32 | 201377_at | NICE-4 | 156,835,350,799,393 | 8.81E−08 | 3.04E−04 | 0.664190316298723 | 107,661,298,383,645 | NICE-4 protein |
| 33 | 209523_at | TAF2 | 170,086,707,324,231 | 3.53E−06 | 1.07E−03 | 0.682592061168908 | 106,972,210,037,855 | TAF2 RNA polymerase II, TATA box |
| | | | | | | | | binding protein (TBP)-associated factor, 150 kDa |
| 34 | 229693_at | | −175,675,663,418,723 | 4.26E−09 | 2.21E−06 | −0.576353171339351 | −1,067,136,757,546 | ESTs |
| 35 | 200066_at-HG- | IK | −142,856,317,950,339 | 3.79E−08 | 1.72E−05 | −0.590633532411712 | −10,607,405,374,437 | IK cytokine, down-regulator of HLA II |
| | U133A | | | | | | | |
| 36 | 236728_at | FLJ39485 | −200,676,202,973,617 | 1.03E−08 | 5.13E−06 | −0.569259101718997 | −105,533,547,236,791 | hypothetical protein FLJ39485 |
| 37 | 212894_at | SUPV3L1 | 141,673,557,040,566 | 8.63E−07 | 3.04E−04 | 0.61987444252067 | 10,523,737,772,068 | suppressor of var1, 3-like 1 (S. cerevisiae) |
| 38 | 224936_at | EIF2S3 | −137,196,707,800,854 | 4.23E−08 | 1.86E−05 | −0.579805173176531 | −105,211,684,205,005 | eukaryotic translation initiation factor 2, subunit 3 |
| | | | | | | | | gamma, 52 kDa |
| 39 | 201222_s_at | RAD23B | 155,900,876,730,256 | 2.24E−06 | 6.92E−04 | 0.621845217538417 | 104,414,587,042,546 | RAD23 homolog B (S. cerevisiae) |
| 40 | 228904_at | | −319,150,465,716,672 | 5.66E−09 | 2.34E−05 | −0.573731733017053 | −104,305,543,620,308 | ESTs |
| 41 | 200023_s_at- | E1F3S5 | −121,999,999,867,377 | 1.19E−06 | 4.02E−04 | −0.592567784154637 | −103,262,409,439,699 | eukaryotic translation initiation factor |
| | HG-U133B | | | | | | | 3, subunit 5 epsilon, 47 kDa |
| 42 | 225326_at | KIAA1311 | −151,260,767,011,645 | 2.20E−07 | 8.64E−05 | −0.569062835222392 | −103,129,285,547,071 | KIAA1311 protein |
| 43 | 224915_x_at | | −142,397,637,904,464 | 1.23E−06 | 4.04E−05 | −0.58028667927814 | −1,024,454,543,165 | Homo sapiens, clone |
| | | | | | | | | IMAGE: 5285034, mRNA |
| 44 | 226227_x_at | | −139,800,457,277,837 | 1.43E−06 | 4.62E−05 | −0.577736346280825 | −102,142,942,870,084 | Homo sapiens, clone |
| | | | | | | | | IMAGE: 5285034, mRNA |
| 45 | 223318_s_at | MGC10974 | −215,038,689,968,521 | 1.63E−07 | 6.58E−05 | −0.550535669133841 | −102,058,126,997,572 | hypothetical protein MGC10974 |
| 46 | 214919_s_at | | −157,916,847,056,614 | 3.04E−07 | 1.16E−04 | −0.553949943054473 | −10,180,703,654,607 | Homo sapiens, clone |
| | | | | | | | | IMAGE: 3866125, mRNA |
| 47 | 222984_at | PAIP2 | −130,364,196,813,178 | 1.60E−07 | 5.05E−04 | −0.568155433373277 | −101,413,895,363,391 | PABP-interacting protein 2 |
| 48 | 208855_s_at | STK24 | 144,316,932,352,991 | 6.36E−06 | 1.84E−03 | 0.584523623741934 | 101,217,526,639,216 | serine/threonine kinase 24 (STE20 homolog, yeast) |
| 49 | 204082_at | PBX3 | −242,166,057,961,334 | 3.36E−07 | 1.25E−04 | −0.545264120738389 | −101,116,484,418,875 | pre-B-cell leukemia transcription factor 3 |
| 50 | 200602_at | APP | 258,324,086,045,963 | 2.23E−05 | 5.68E−03 | 0.604177140166656 | 101,105,526,849,876 | amyloid beta (A4) precursor protein |
| | | | | | | | | (protease nexin-II, Alzheimer disease) |

| # | MapLocation | Sequence Type | Go_Biological_Process | Go_Cellular_Component |
|---|---|---|---|---|
| 1 | 4q12 | Exemplarsequence | | |
| 2 | | Consensussequence | | |
| 3 | 5q31.3 | Exemplarsequence | | |
| 4 | | Consensussequence | | |
| 5 | 5q31.2 | Consensussequence | GO: 7165; signal transduction; traceable author statement | GO: 5634; nucleus; traceable author statement |
| | | | | GO: 5856; cytoskeleton; traceable author statement |
| 6 | 5q31.2 | Exemplarsequence | GO: 7165; signal transduction; traceable author | GO: 5634; nucleus; traceable author statement |
| | | | statement | GO: 5856; cytoskeleton; traceable author statement |
| 7 | 5q31.2 | Consensussequence | GO: 7165; signal transduction; traceable author statement | GO: 5634; nucleus; traceable author statement |
| | | | | GO: 5856; cytoskeleton; traceable author statement |
| 8 | 5q31 | Exemplarsequence | | |
| 9 | 7q31.1 | Exemplarsequence | | |
| 10 | 5q13.2 | Exemplarsequence | GO: 6412; protein biosynthesis; inferred from electronic | GO: 5840; ribosome; inferred from electronic annotation |
| | | | annotation | GO: 5634; nucleus; inferred from electronic annotation |
| 11 | 5q14 | Consensussequence | GO: 6118; electron transport; inferred from electronic | GO: 5739; mitochondrion; inferred from electronic annotation |
| | | | annotation GO: 6091; energy pathways; traceable | |
| | | | author statement | |
| 12 | | Consensussequence | | |
| 13 | 5q31.1 | Exemplarsequence | GO: 6449; regulation of translational | GO: 5737; cytoplasm; traceable author statement |
| | | | termination; traceable author statement | |
| 14 | 5q33.1 | Exemplarsequence | | |
| 15 | | Consensussequence | | |
| 16 | 17q21.3 | Exemplarsequence | GO: 6355; regulation of transcription, DNA- | GO: 5634; nucleus; non-traceable author statement |
| | | | dependent; non-traceable author statement | |
| | | | GO: 7275; development; inferred from electronic | |
| | | | annotation GO: 8595; determination of | |
| | | | anterior/posterior axis, embryo; non-traceable author | |
| | | | statement | |
| 17 | 5q31.3 | Exemplarsequence | | |
| 18 | 19p13.3 | Exemplarsequence | GO: 6508; proteolysis and peptidolysis; traceable author | |
| | | | statement GO: 6957; complement activation, alternative | |
| | | | pathway: inferred from electronic annotation | |
| 19 | 5q31.3 | Exemplarsequence | GO: 6464; protein modification; traceable author | |
| | | | statement GO: 6512; ubiquitin cycle; inferred from | |
| | | | electronic annotation GO: 7125; invasive | |
| | | | growth; traceable author statement | |
| | | | GO: 7048; oncogenesis; traceable author statement | |
| 20 | 1p32.1-p31.3 | Exemplarsequence | GO: 6511; ubiquitin-dependent protein | |
| | | | catabolism; inferred from electronic annotation | |
| 21 | 7q34 | Exemplarsequence | | GO: 5739; mitochondrion; inferred from electronic annotation |
| | | | | GO: 30288: periplasmic space (sensu Gram-negative |
| | | | | Becteria); inferred from electronic annotation |
| 22 | 5q31.2 | Exemplarsequence | GO: 7165; signal transduction; traceable author | GO: 5634; nucleus; traceable author statement |
| | | | statement | GO: 5856; cytoskeleton; traceable author statement |
| 23 | | Consensussequence | | |
| 24 | 8q24 | Exemplarsequence | GO: 6302; double-strand break repair; traceable author | GO: 5634; nucleus; inferred from electronic annotation |
| | | | statement GO: 7131; melotic recombination; traceable | |
| | | | author statement GO: 7049; cell cycle; inferred from | |
| | | | electronic annotation GO: 7067; mitosis: inferred from | |
| | | | electronic annotation GO: 7059; chromosome | |
| | | | segregation; inferred from electronic annotation | |
| | | | GO: 6915; apoptosis; inferred from electronic annotation | |
| 25 | 5q31.3 | Consensussequence | | |
| 26 | 5q23 | Exemplarsequence | GO: 7165; signal transduction; traceable author | GO: 16021; integral to membrane; inferred from electronic |
| | | | statement GO: 6952; defense response; traceable | annotation |
| | | | author statement | |
| 27 | 5q31-q33 | Exemplarsequence | GO: 6412; protein biosynthesis; predicted/computed | GO: 5843; cytosolic small ribosomal subunit (sensu |
| | | | | Eukarya); predicted/computed |
| 28 | 1p34.1 | Consensussequence | | |
| 29 | | Consensussequence | | |
| 30 | 5q31.3 | Exemplarsequence | GO: 7267; cell-cell signaling; traceable author statement | GO: 5625; soluble fraction; traceable author statement |
| | | | GO: 6955; immune response; traceable author | GO: 5615; extracellular space; traceable author statement |
| | | | statement | |
| 31 | 3p24.1 | Exemplarsequence | GO: 6412; protein biosynthesis; traceable author | GO: 5840; ribosome; traceable author statement |
| | | | statement | GO: 5622; intracellular; inferred from electronic annotation |
| 32 | 1q21.3 | Exemplarsequence | | |
| 33 | 8q24.12 | Consensussequence | GO: 6508; proteolysis and peptidolysis; inferred from | |
| | | | electronic annotation | |
| 34 | | Consensussequence | | |
| 35 | 5q31.3 | Exemplarsequence | GO: 7267; cell-cell signaling; traceable author statement | GO: 5625; soluble fraction; traceable author statement |
| | | | GO: 6955; immune response; traceable author | GO: 5615; extracellular space; traceable author statement |
| | | | statement | |
| 36 | 5q14.3 | Consensussequence | | |
| 37 | 10q22.1 | Consensussequence | | |
| 38 | Xp22.2-p22.1 | Consensussequence | GO: 6414; translational elongation; inferred from | GO: 5843; cytosolic small ribosomal subunit (sensu |
| | | | electronic annotation | Eukarya); not recorded GO: 5850; eukaryotic translation initiation |
| | | | | factor 2 complex; not recorded |
| 39 | 9q31.2 | Consensussequence | GO: 6289; nucleotide-excision repair; experimental | GO: 5634; nucleus; predicted/computed |
| | | | evidence | |
| 40 | | Consensussequence | | |
| 41 | 11p15.3 | Exemplarsequence | GO: 6446; regulation of translational initiation; traceable | GO: 5852; eukaryolic translation initiation factor 3 |
| | | | author statement | complex; traceable author statement |
| 42 | 5q32 | Consensussequence | | |
| 43 | | Consensussequence | | |
| 44 | | Consensussequence | | |
| 45 | 19p13.3 | Exemplarsequence | | |
| 46 | | Consensussequence | | |
| 47 | 5q31.3 | Exemplarsequence | | |
| 48 | 13q31.2-q32.3 | Exemplarsequence | GO: 7165; signal transduction; traceable author | |
| | | | statement GO: 6468; protein amino acid | |
| | | | phosphorylation; inferred from electronic annotation | |
| 49 | 9q33-q34 | Exemplarsequence | GO: 7388; posterior compartment | |
| | | | specification; predicted/computed GO: 7387; anterior | |
| | | | compartment specification; predicted/computed | |
| | | | GO: 7048; oncogenesis; predicted/computed | |
| 50 | 21q21.3 | Exemplarsequence | GO: 7165; signal transduction; traceable author | GO: 16020; A4_EXTRA; membrane; 1.8e−130; extended: Unkonwn |
| | | | statement GO: 6897; endocytosis; inferred from | GO: 5783; endoplasmic reticulum; traceable author statement |
| | | | electronic annotation GO: 6878; copper ion | GO: 5576; extracellular; traceable author statement |
| | | | homeostasis; traceable author statement GO: 8219; cell | GO: 5887; integral to plasma membrane; traceable author |
| | | | death; traceable author statement | statement GO: 5905; coated pit; inferred from electronic |
| | | | GO: 6915; apoptosis; traceable author statement | annotation GO: 5794; Golgai apparatus; traceable author |
| | | | GO: 7155; cell adhesion; inferred from electronic | statement |
| | | | annotation | |

| # | Go_Molecular_Function | Transcript ID | Sequence Derived From | Sequence ID | Sequence Source | Unigene_Accession |
|---|---|---|---|---|---|---|
| 1 | | Hs.8715.0 | BC004894.1 | g13436154 | GenBank | Hs.8715 |
| 2 | | Hs.63510.1 | AA181172 | Hs.63510.1.A1 | GenBank | Hs.446502 |
| 3 | | Hs.108548.0 | BC001716.1 | g12804590 | GenBank | Hs.396644 |
| 4 | | Hs.272344.0 | AL121871 | Hs.272344.0 | GenBank | |
| 5 | GO: 16787; hydrolase activity; inferred from electronic annotation GO: 5080; protein kinase C | Hs.256697.2 | N32864 | Hs.256697.2.A1 | GenBank | Hs.256697 |
| | binding; traceable author statement GO: 8270; zinc ion binding; not recorded | | | | | |
| 6 | GO: 16787; hydrolase activity; inferred from electronic annotation GO: 5080; protein kinase C | Hs.256697.1 | U27143.1 | g862932 | GenBank | Hs.256697 |
| | binding; traceable author statement GO: 8270; zinc ion binding; not recorded | | | | | |
| 7 | GO: 16787; hydrolase activity; inferred from electronic annotation GO: 5080; protein kinase C | Hs.256697.2 | N32864 | Hs.256697.2.A1 | GenBank | Hs.256697 |
| | binding; traceable author statement GO: 8270; zinc ion binding; not recorded | | | | | |
| 8 | | Hs.297875.0 | NM_022464.1 | g11968008 | RefSeq | Hs.297875 |
| 9 | | Hs.42636.0 | NM_021994.1 | g11496268 | RefSeq | Hs.42636 |
| 10 | GO: 3735; structural constituent of ribosome; inferred from electronic annotation | Hs.8170.0 | NM_014886.1 | g7662676 | RefSeq | Hs.8170 |
| 11 | GO: 9482; ba3-type cytochrome c oxidase; inferred from electronic annotation GO: 9481; aa3- | Hs.3462.1 | AA382702 | Hs.3462.1 | GenBank | Hs.430075 |
| | type cytochrome c oxidase; inferred from electronic annotation GO: 9483; caa3-type | | | | | |
| | cytochrome c oxidase; inferred from electronic annotation GO: 4129; cytochrome c oxidase | | | | | |
| | activity; inferred from electronic annotation GO: 16491; oxidoreductase activity; inferred from | | | | | |
| | electronic annotation GO: 9485; cbb3-type cytochrome c oxidase; inferred from electronic | | | | | |
| | annotation | | | | | |
| 12 | | Hs.269918.0 | BF590528 | Hs.269918.0.A1 | GenBank | Hs.183096 |
| 13 | GO: 16149; translation release factor activity, codon specific; inferred from electronic | Hs.77324.0 | NM_004730.1 | g4759033 | RefSeq | Hs.77324 |
| | annotation GO: 3723; RNA binding; traceable author statement | | | | | |
| 14 | | Hs.29444.0 | AF313413.1 | g12484085 | GenBank | Hs.29444 |
| 15 | | Hs.269918.1 | AI125255 | Hs.269918.1.A1 | GenBank | Hs.183096 |
| 16 | GO: 3700; transcription factor activity; non-traceable author statement | Hs.98428.0 | NM_018952.1 | g9506792 | RefSeq | Hs.98428 |
| 17 | | Hs.63510.0 | NM_014773.1 | g7661939 | RefSeq | Hs.63510 |
| 18 | GO: 3817; complement factor D activity; traceable author statement GO: 16787; hydrolase | Hs.155597.0 | NM_001928.1 | g4503308 | RefSeq | Hs.155597 |
| | activity; inferred from electronic annotation GO: 4295; trypsin activity; inferred from electronic | | | | | |
| | annotation GO: 4263; chymotrypsin activity; inferred from electronic annotation | | | | | |
| 19 | GO: 16874; ligase activity; inferred from electronic annotation GO: 4842; ubiquitin-protein | Hs.108332.0 | NM_003339.1 | g4507774 | RefSeq | Hs.108332 |
| | ligase activity; traceable author statement GO: 4840; ubiquitin conjugating enzyme | | | | | |
| | activity, traceable author statement | | | | | |
| 20 | GO: 4197; cysteine-type endopeptidase activity; traceable author statement | Hs.35086.0 | NM_003368.1 | g4507850 | RefSeq | Hs.35086 |
| | GO: 16787; hydrolase activity; inferred from electronic annotation GO: 4221; ubiquthin C- | | | | | |
| | terminal hydrolase activity; inferred from electronic annotation GO: 4843; ubiquitin-specific | | | | | |
| | protease activity; traceable author statement | | | | | |
| 21 | GO: 4364; glutathione transferase activity; inferred from electronic annotation | Hs.279952.0 | NM_015917.1 | g7705703 | RefSeq | Hs.279952 |
| | GO: 16740; transferase activity; inferred from electronic annotation GO: 15035; protein | | | | | |
| | disulfide oxidoreductase activity; inferred from electronic annotation | | | | | |
| 22 | GO: 16787; hydrolase activity; inferred from electronic annotation GO: 5080; protein kinase C | Hs.256697.0 | NM_005340.1 | g4885412 | RefSeq | Hs.256697 |
| | binding; traceable author statement GO: 8270; zinc ion binding; not recorded | | | | | |
| 23 | | Hs.262036.0 | AL577866 | Hs.262036.0.S1 | GenBenk | Hs.262036 |
| 24 | | Hs.81848.0 | NM_006265.1 | g5453993 | RefSeq | Hs.81848 |
| 25 | | Hs.63510.0 | AI539425 | Hs.63510.0.A1 | GenBank | Hs.63510 |
| 26 | | Hs.13572.0 | NM_001745.1 | g4502558 | RefSeq | Hs.13572 |
| 27 | GO: 3735; structural protein of ribosome; predicted/computed GO: 3723; RNA | Hs.244621.0 | AF116710.1 | g7959918 | GenBank | Hs.431584 |
| | binding; predicted/computed | | | | | |
| 28 | | Hs.59584.0 | AU144258 | Hs.59584.0.S1 | GenBank | Hs.59584 |
| 29 | | Hs.284158.3 | BG330520 | Hs.284158.3 | GenBank | Hs.356766 |
| 30 | GO: 5125; cytokine activity; not recorded | Hs.8024.0 | AF182645.1 | g5901877 | GenBank | Hs.8024 |
| 31 | GO: 3723; RNA binding; traceable author statement GO: 3735; structural constituent of | Hs.74267.0 | AF279903.1 | g12006349 | GenBank | Hs.74267 |
| | ribosome; traceable author statement | | | | | |
| 32 | | Hs.8127.0 | NM_014847.1 | g7661941 | RefSeq | Hs.8127 |
| 33 | GO: 4179; membrane alanyl aminopeptidase activity; inferred from electronic annotation | Hs.122752.0 | AK001618.1 | Hs.122752.0 | GenBank | Hs.122752 |
| 34 | | Hs.154574.0 | AI952836 | Hs.154574.0_RC | GenBank | Hs.154574 |
| 35 | GO: 5125; cytokine activity; not recorded | Hs.8024.0 | AF182645.1 | g5901877 | GenBank | Hs.8024 |
| 36 | | Hs.26330.0 | AW070437 | Hs.26330.0.A1 | GenBank | Hs.391318 |
| 37 | GO: 8026; ATP dependent helicase activity; inferred from electronic annotation | Hs.106469.0 | NM_003171.1 | Hs.106469.0 | GenBank | Hs.106469 |
| | GO: 3723; RNA binding; traceable author statement GO: 16787; hydrolase activity; interred | | | | | |
| | from electronic annotation GO: 3676; helicase_C; nucleic acid binding activity; 7.9e−17; | | | | | |
| | extended: inferred from electronic annotation GO: 5524; ATP binding; inferred from | | | | | |
| | electronic annotation | | | | | |
| 38 | GO: 3743; translation initiation factor activity; inferred from electronic annotation | Hs.30376.1 | BE252813 | Hs.30376.1.A1 | GenBank | Hs.211539 |
| | GO: 3746; translation elongation factor activity; inferred from electronic annotation | | | | | |
| | GO: 3924; GTPase activity; traceable author statement | | | | | |
| 39 | GO: 3697; single-stranded DNA binding; experimental evidence | Hs.178658.0 | AL527365 | Hs.178658.0.S1 | GenBank | Hs.404283 |
| 40 | | Hs.156044.0 | AW510657 | Hs.156044.0 | GenBank | Hs.156044 |
| 41 | GO: 3743; translation initiation factor activity; traceable author statement | Hs.7811.0 | NM_003754.1 | g4503518 | RefSeq | Hs.7811 |
| 42 | GO: 3676; nucleic acid binding; inferred from electronic annotation | Hs.61441.0 | AB037732.1 | Hs.61441.0.S1 | GenBank | Hs.61441 |
| 43 | | Hs.284158.1 | AV756131 | Hs.284158.1_RC | GenBank | Hs.356766 |
| 44 | | Hs.284158.4 | BF185165 | Hs.284158.4 | GenBank | Hs.356766 |
| 45 | | Hs.111099.0 | BC004393.1 | g13325151 | GenBank | Hs.111099 |
| 46 | | Hs.301226.2 | R39094 | Hs.301226.2.A1 | GenBank | Hs.301226 |
| 47 | | Hs.108548.0 | AF151052.1 | g7106825 | GenBank | Hs.396644 |
| 48 | GO: 4672; pkinase; protein kinase activity; 5.8e−91; extended: inferred from electronic | Hs.168913.1 | AF083420.1 | g5326765 | GenBank | Hs.168913 |
| | annotation GO: 4691; cAMP-dependent protein kinase activity; inferred from electronic | | | | | |
| | annotation GO: 4713; protein tyrosine kinase activity; inferred from electronic annotation | | | | | |
| | GO: 16740; transferase activity; inferred from electronic annotation GO: 5524; ATP | | | | | |
| | binding; inferred from electronic annotation GO: 4682; protein kinase CK2 activity; inferred | | | | | |
| | from electronic annotation GO: 4674; protein serine/threonine kinase activity; inferred from | | | | | |
| | electronic annotation | | | | | |
| 49 | GO: 3677; DNA binding; not recorded GO: 3700; PBX; transcription factor activity; 8.9e−147; | Hs.294101.0 | NM_006195.1 | g5453851 | RefSeq | Hs.294101 |
| | extended: inferred from electronic annotation | | | | | |
| 50 | GO: 4867; serine protease inhibitor activity; traceable author statement GO: 5515; protein | Hs.177486.0 | NM_000484.1 | g4502166 | RefSeq | Hs.177486 |
| | binding; inferred from physical interaction GO: 8201; heparin binding; inferred from electronic | | | | | |
| | annotation GO: 5194; cell adhesion molecule activity; inferred from electronic annotation | | | | | |

| # | Cluster_Type | LocusLink | Full_Length_Reference_Seq |
|---|---|---|---|
| 1 | fulllength | 84273 | NM_032313; hypothetical protein |
| | | | MGC3232 |
| 2 | est | | |
| 3 | fulllength | 51247 | NM_016480; polyadenylate-binding protein- |
| | | | interacting protein 2 |
| 4 | | | |
| 5 | fulllength | 3094 | NM_005340; histidine triad nucleotide |
| | | | binding protein 1 |
| 6 | fulllength | 3094 | NM_005340; histidine triad nucleotide |
| | | | binding protein 1 |
| 7 | fulllength | 3094 | NM_005340; histidine triad nucleotide |
| | | | binding protein 1 |
| 8 | fulllength | 64374 | NM_022464; endoplasmic reticulum |
| | | | chaperone SIL1, homolog of yeast |
| 9 | fulllength | 11179 | NM_021994; zinc finger protein (C2H2 |
| | | | type) 277 |
| 10 | fulllength | 10412 | NM_014886; TGF beta-inducible nuclear |
| | | | protein 1 |
| 11 | fulllength | 1350 | NM_001867; cytochrome c oxidase subunit |
| | | | VIIc precursor |
| 12 | fulllength | | |
| 13 | fulllength | 2107 | NM_004730; eukaryotic translation |
| | | | termination factor 1 |
| 14 | fulllength | 85027 | NM_032947; putative small membrane |
| | | | protein NID67 |
| 15 | fulllength | | |
| 16 | fulllength | 3216 | NM_018952; homeo box B6 isoform 1 |
| | | | NM_156036; homeo box B6 isoform 2 |
| | | | NM_156037; homeo box B6 isoform 1 |
| 17 | fulllength | 9812 | NM_014773; KIAA0141 gene product |
| 18 | fulllength | 1675 | NM_001928; adipsin/complement factor D |
| | | | precursor |
| 19 | fulllength | 7322 | NM_003339; ubiquitin-conjugating enzyme |
| | | | E2D 2 (UBC4/5 homolog, yeast) |
| 20 | fulllength | 7398 | NM_003368; ubiquitin specific protease 1 |
| 21 | fulllength | 51064 | NM_015917; glutathione S-transferase |
| | | | subunit 13 homolog |
| 22 | fulllength | 3094 | NM_005340; histidine triad nucleotide |
| | | | binding protein 1 |
| 23 | | | |
| 24 | fulllength | 5885 | NM_006265; RAD21 homolog |
| 25 | fulllength | 9812 | NM_014773; KIAA0141 gene product |
| 26 | fulllength | 819 | NM_001745; calcium modulating ligand |
| 27 | fulllength | 6208 | NM_005617; ribosomal protein S14 |
| 28 | fulllength | 64789 | NM_022774; hypothetical protein FLJ21144 |
| 29 | | | |
| 30 | fulllength | 3550 | NM_004511; NM_006083; RED protein |
| 31 | fulllength | 6138 | NM_002948; ribosomal protein L15 |
| 32 | fulllength | 9898 | NM_014847; NICE-4 protein |
| 33 | fulllength | 6873 | NM_003184; TBP-associated factor 2 |
| 34 | est | | |
| 35 | fulllength | 3550 | NM_004511; NM_006083; RED protein |
| 36 | | 285603 | NM_175920; hypothetical protein FLJ39485 |
| 37 | fulllength | 6832 | NM_003171; suppressor of var1, 3-like 1 |
| 38 | fulllength | 1968 | NM_001415; eukaryotic translation |
| | | | initiation factor 2, subunit 3 gamma, 52 kDa |
| 39 | fulllength | 5887 | NM_002874; UV excision repair protein |
| | | | RAD23 homolog B |
| 40 | est | | |
| 41 | fulllength | 8665 | NM_003754; eukaryotic translation |
| | | | initiation factor 3, subunit 5 epsilon, 47 kDa |
| 42 | | 54439 | |
| 43 | | | |
| 44 | | | |
| 45 | fulllength | 84266 | NM_032306; hypothetical protein |
| | | | MGC10974 |
| 46 | fulllength | | NM_003732; eukaryotic translation |
| | | | initiation factor 4E binding protein 3 |
| 47 | fulllength | 51247 | NM_016480; polyadenylale-binding protein- |
| | | | interacting protein 2 |
| 48 | fulllength | 8428 | NM_003576; serine/threonine kinase 24 |
| | | | (STE20 homolog, yeast) |
| 49 | fulllength | 5090 | NM_006195; pre-B-cell leukemia |
| | | | transcription factor 3 |
| 50 | fulllength | 351 | NM_000484; amyloid beta (A4) precursor |
| | | | protein (protease nexin-II, Alzheimer |
| | | | disease) |

1. A method of classifying an acute myeloid leukemia (AML) cell, the method comprising:
detecting an expression level of at least one set of genes in or derived from at least one target AML cell; and,
correlating a detected differential expression of one or more genes selected from the markers listed in one or more of Tables 1-13 relative to a corresponding expression of the genes in or derived from at least one reference AML cell having a reciprocal translocation with the target AML cell having a CEBPA mutation;
correlating a detected substantially identical expression of one or more genes selected from the markers listed in one or more of Tables 1-13 relative to a corresponding expression of the genes in or derived from at least one reference AML cell having a CEBPA mutation with the target AML cell having the CEBPA mutation;
correlating a detected differential expression of one or more genes selected from the markers listed in one or more of Tables 1-13 relative to a corresponding expression of the genes in or derived from at least one reference AML cell having a CEBPA mutation with the target AML cell having a reciprocal translocation; or,
correlating a detected substantially identical expression of one or more genes selected from the markers listed in one or more of Tables 1-13 relative to a corresponding expression of the genes in or derived from at least one reference AML cell having a reciprocal translocation with the target AML cell having the reciprocal translocation,
thereby classifying the AML cell.

2. The method of claim 1, wherein the target AML cell comprises an intermediate karyotype.

3. The method of claim 1, wherein the detected differential or substantially identical expression expression comprises one or more of the markers listed in Table 3 and/or Table 4 when the reciprocal translocation comprises a t(11q23).

4. The method of claim 1, wherein the detected differential or substantially identical expression expression comprises one or more of the markers listed in Table 5 and/or Table 6 when the reciprocal translocation comprises an inv(16).

5. The method of claim 1, wherein the detected differential or substantially identical expression expression comprises one or more of the markers listed in Table 7 and/or Table 8 when the reciprocal translocation comprises an inv(3).

6. The method of claim 1, wherein the detected differential or substantially identical expression expression comprises one or more of the markers listed in Table 9 and/or Table 10 when the reciprocal translocation comprises a t(8;21).

7. The method of claim 1, wherein the detected differential or substantially identical expression expression comprises one or more of the markers listed in Table 11 and/or Table 12 when the reciprocal translocation comprises a t(15;17).

8. The method of claim 1, comprising:
correlating a detected higher expression of an MPO gene from the target AML cell having a CEBPA mutation, and/or a detected lower expression of one or more of: a HOXA3 gene, a HOXA7 gene, a HOXA9 gene, a HOXB4 gene, a HOXB6 gene, or a PBX3 gene from the target AML cell having the CEBPA mutation, relative to at least one reference AML cell lacking the CEBPA mutation with the target AML being a group A AML cell; or,
correlating a detected lower expression of an MPO gene from the target AML cell having a CEBPA mutation, and/or a detected higher expression of one or more of: a HOXA3 gene, a HOXA7 gene, a HOXA9 gene, a HOXB4 gene, a HOXB6 gene, and a PBX3 gene from the target AML cell having the CEBPA mutation, relative to at least one reference AML cell lacking the CEBPA mutation with the target AML being a group B AML cell.

9. The method of claim 1, wherein the set of genes in or derived from the target AML cell comprises at least about 10, 100, 1000, 10000, or more members.

10. The method of claim 1, wherein the target AML cell is obtained from a subject.

11. The method of claim 1, wherein the detected differential expression of the genes comprises at least about a 5% difference.

12. The method of claim 1, wherein the detected substantially identical expression of the genes comprises less than about a 5% difference.

13. The method of claim 1, wherein the expression level is detected using an array, a robotics system, and/or a microfluidic device.

14. The method of claim 1, wherein the expression level of the set of genes is detected by amplifying nucleic acid sequences associated with the genes to produce amplicons and detecting the amplicons.

15. The method of claim 14, wherein the amplicons are detected using a process that comprises one or more of: hybridizing the amplicons to an oligonucleotide array, digesting the amplicons with a restriction enzyme, or real-time polymerase chain reaction (PCR) analysis.

16. The method of claim 1, wherein detecting the expression level of the set of genes comprises measuring quantities of transcribed polynucleotides or portions thereof expressed or derived from the genes.

17. The method of claim 16, wherein the transcribed polynucleotides are mRNAs or cDNAs.

18. The method of claim 1, wherein detecting the expression level comprises contacting polynucleotides or polypeptides expressed from the genes with compounds that specifically bind the polynucleotides or polypeptides.

19. The method of claim 18, wherein the compounds comprise aptamers, antibodies or fragments thereof.

20. A method of producing a reference data bank for classifying AML cells, the method comprising:
(a) compiling a gene expression profile of a patient sample by detecting the expression level of one or more genes of at least one AML cell, which genes are selected from the markers listed in one or more of Tables 1-13, and;
(b) classifying the gene expression profile using a machine learning algorithm.

21. The reference data bank produced by the method of claim 20.

22. A kit, comprising:
one or more probes that correspond to at least portions of genes or expression products thereof, which genes are selected from the markers listed in one or more of Tables 1-13; and,
instructions for correlating detected expression levels of polynucleotides and/or polypeptides in at least one target cell from a subject, which polynucleotides and/or polypeptides are targets of one or more of the probes, with the target cell being an AML cell having a CEBPA mutation or a reciprocal translocation.

23. The kit of claim 22, wherein at least one solid support comprises the probes.

24. The kit of claim 22, comprising one or more additional reagents to perform real-time PCR analyses.

25. A system, comprising:
one or more probes that correspond to at least portions of genes or expression products thereof, which genes are selected from the markers listed in one or more of Tables 1-17; and,
at least one reference data bank for correlating detected expression levels of polynucleotides and/or polypeptides in at least one target cell from a subject, which polynucleotides and/or polypeptides are targets of one or more of the probes, with the target cell being an AML cell having a CEBPA mutation or a reciprocal translocation.

26. The system of claim 25, wherein at least one solid support comprises the probes.

27. The system of claim 25, comprising one or more additional reagents and/or components to perform real-time PCR analyses.

28. The system of claim 25, wherein the reference data bank is produced by:
(a) compiling a gene expression profile of a patient sample by determining the expression level at least one of the genes, and
(b) classifying the gene expression profile using a machine learning algorithm.

29. The system of claim 28, wherein the machine learning algorithm is selected from the group consisting of: a weighted voting algorithm, a K-nearest neighbors algorithm, a decision tree induction algorithm, a support vector machine, and a feed-forward neural network.

30. A method of aiding in a leukemia prognosis for a subject, the method comprising:
detecting an expression level of at least one set of genes in or derived from at least one target acute myeloid leukemia (AML) cell from the subject; and,
correlating a detected a higher expression of an MPO gene and/or an ATBF1 gene in the target AML cell relative to a corresponding expression of the genes in or derived from an AML cell from a member of an unfavorable group with the subject having a probable overall survival rate at three years of about 55% or more; or,
correlating a detected a higher expression of one or more of: an ETS2 gene, a RUNX1 gene, a TCF4 gene, a FOXC1 gene, a SFRS1 gene, a TPD52 gene, a NRIP1 gene, a TFPI gene, a UBL1 gene, an REC8L1 gene, an HSF2 gene, or an ETS2 gene in the target AML cell relative to a corresponding expression of the genes in or derived from an AML cell from a member of a favorable group with the subject having a probable overall survival rate at three years of about 25% or less,
thereby aiding in the leukemia prognosis for the subject.

31-41. (canceled)

42. A method of producing a reference data bank for aiding in leukemia prognostication, the method comprising:
(a) compiling a gene expression profile of a patient sample by determining the expression level at least one marker selected from: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker, and;
(b) classifying the gene expression profile using a machine learning algorithm.

43. The reference data bank produced by the method of claim 42.

44. A kit, comprising:
one or more markers or portions thereof selected from the group consisting of: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker; and,
instructions for correlating detected expression levels of polynucleotides and/or polypeptides in at least one target AML cell from a subject, which polynucleotides and/or polypeptides correspond to one or more of the markers, with a probable overall survival rate for the subject.

45-48. (canceled)

49. A system, comprising:
one or more markers or portions thereof selected from the group consisting of: an MPO marker, an ATBF1 marker, an ETS2 marker, a RUNX1 marker, a TCF4 marker, a FOXC1 marker, a SFRS1 marker, a TPD52 marker, a NRIP1 marker, a TFPI marker, a UBL1 marker, an REC8L1 marker, an HSF2 marker, and an ETS2 marker; and,
at least one reference data bank for correlating detected expression levels of polynucleotides and/or polypeptides in target AML cells, which polynucleotides and/or polypeptides correspond to one or more of the markers, with a probable overall survival rate for a subject.

50-51. (canceled)

52. A method of detecting acute myeloid leukemia (AML) with t(8;16), the method comprising:
detecting an expression level of at least one set of genes in or derived from at least one target AML cell; and,
correlating a detected differential expression of one or more genes of the target AML cell relative to a corresponding expression of the genes in or derived from a reference AML cell with t(15;17), t(8;21), inv(16), or 11q23/MLL with the target AML cell being a target AML cell with t(8;16); or,
correlating a detected substantially identical expression of one or more genes of the target AML cell relative to a corresponding expression of the genes in or derived from a reference AML cell with t(8;16) with the target AML cell being a target AML cell with t(8;16), thereby detecting AML with t(8;16).

53-66. (canceled)

67. A method of producing a reference data bank for identifying AML cells with t(8;16), the method comprising:
(a) compiling a gene expression profile of a patient sample by determining the expression level of one or more genes of at least one AML cell, which genes are selected from the markers listed in Table 18, and;
(b) classifying the gene expression profile using a machine learning algorithm.

68. The reference data bank produced by the method of claim 67.

69. A kit, comprising:
one or more probes that correspond to at least portions of genes or expression products thereof, which genes are selected from the markers listed in Table 18; and,
instructions for correlating detected expression levels of polynucleotides and/or polypeptides in at least one target AML cell from a human subject, which polynucleotides and/or polypeptides are targets of one or more of the probes, with the target AML cell comprising t(8;16).

70-71. (canceled)

72. A system, comprising:
one or more probes that correspond to at least portions of genes or expression products thereof, which genes are selected from the markers listed in Table 18; and,
at least one reference data bank for correlating detected expression levels of polynucleotides and/or polypeptides in target human AML cells, which polynucleotides and/or polypeptides are targets of one or more of the probes, with the target AML cells comprising t(8;16).

73-76. (canceled)

77. A method of identifying an acute myeloid leukemia (AML) cell comprising trisomy 8, the method comprising:
(a) detecting an expression level of at least one set of genes in or derived from at least one target human AML cell; and,
(b) correlating a detected differential expression of one or more genes of the target human AML cell relative to a corresponding expression of the genes in or derived from a human AML cell lacking trisomy 8 with the target human AML cell comprising trisomy 8; or,
(c) correlating a detected substantially identical expression of one or more genes of the target human AML cell relative to a corresponding expression of the genes in or derived from a human AML cell comprising trisomy 8 with the target human AML cell comprising trisomy 8, thereby identifying the AML cell comprising trisomy 8.

78-85. (canceled)

86. A method of classifying a cell, the method comprising:
detecting an expression level of at least one set of genes in or derived from at least one target cell; and,
correlating a detected differential expression of one or more genes of the target cell relative to a corresponding expression of the genes in or derived from an acute myeloid leukemia (AML) cell with the target cell being a myelodysplastic syndrome (MDS) cell; or
correlating a detected substantially identical expression of one or more genes of the target cell relative to a corresponding expression of the genes in or derived from an AML cell with the target cell being an AML cell; or
correlating a detected differential expression of one or more genes of the target cell relative to a corresponding expression of the genes in or derived from an MDS cell with the target cell being an AML cell; or
correlating a detected substantially identical expression of one or more genes of the target cell relative to a corresponding expression of the genes in or derived from an MDS cell with the target cell being an MDS cell, thereby classifying the cell.

87-97. (canceled)

98. A method of subclassifying an acute myeloid leukemia-normal karyotype (AML-NK) cell, the method comprising:
detecting an expression level of at least one set of genes in or derived from at least one target AML-NK cell; and,
correlating:
a detected higher expression of one or more genes selected from the group listed in Table 38 and/or a detected lower expression of one or more genes selected from the group listed in Table 39 of the target AML-NK cell relative to a corresponding expression of the genes in or derived from a Group B AML-NK cell with the target AML-NK cell being a Group A AML-NK cell; or
a detected lower expression of one or more genes selected from the group listed in Table 38 and/or a detected higher expression of one or more genes selected from the group listed in Table 39 of the target AML-NK cell relative to a corresponding expression of the genes in or derived from a Group A AML-NK cell with the target AML-NK cell being a Group B AML-NK cell, thereby subclassifying the AML-NK cell.

99. A method of identifying a cell with a 5q deletion ((del)5q), the method comprising:
detecting an expression level of at least one set of genes in or derived from at least one target human cell; and,
correlating a detected differential expression of one or more genes of at least chromosome 5 of the target human cell relative to a corresponding expression of the genes in or derived from a human cell lacking a (del)5q with the target human cell comprising a (del)₅q; or,
correlating a detected substantially identical expression of one or more genes of at least chromosome 5 of the target human cell relative to a corresponding expression of the genes in or derived from a human cell having a (del)5q with the target human cell comprising a (del)5q, thereby identifying the cell with the (del)5q.

100-105. (canceled)
